(19) United States
(12) Patent Application Publication
(10) Pub. No.: US 2024/0383875 A1
(43) Pub. Date: Nov. 21, 2024

(54) Benzimidazole Pyridine Derivatives

(71) Applicant: Hoffmann-La Roche Inc., Little Falls, NJ (US)

(72) Inventors: Mahendra AWALE, Basel (CH); Stefan BERCHTOLD, Binningen (CH); Julie CHARPENTIER, Basel (CH); Héloïse Marie Albine COLOMBANO, Zuerich (CH); Guillaume DECORET, Eschentzwiller (FR); Katrin GROEBKE ZBINDEN, Liestal (CH); Nicole GROSSMANN, Sisseln (CH); Wolfgang HAAP, Lörrach (DE); Philip Anthony HARRIS, Cambridge, MA (US); Jérôme HERT, Basel (CH); Jonah Milton KALLENBACH, Cambridge, MA (US); Christian KRAMER, Lörrach (DE); Lukas KREIS, Baden (CH); Danny KRUMM, Basel (CH); Xavier LUCAS CABRE, Basel (CH); Nenad MANEVSKI, Riehen (CH); Philippe PFLIEGER, Schwoben (FR); Amir Mohsen POURMOUSA ABKENAR, Cambridge, MA (US); Etienne RAUBER, Hundsbach (FR); Dazhi TAN, Cambridge, MA (US); Jean-Yves WACH, Basel (CH); Roger WERMUTH, Sissach (CH)

(73) Assignee: Hoffmann-La Roche Inc., Little Falls, NJ (US)

(21) Appl. No.: 18/777,339

(22) Filed: Jul. 18, 2024

Related U.S. Application Data
(63) Continuation of application No. PCT/EP2023/051059, filed on Jan. 18, 2023.

(30) Foreign Application Priority Data
Jan. 19, 2022 (EP) 22152225.3

(51) Int. Cl. C07D 401/14; A61K 9/00; A61K 9/20; A61K 9/28; A61K 9/48; A61K 31/4439; A61K 31/4545; A61K 31/496; A61K 31/497; A61K 31/501; A61K 31/506; A61K 31/53; A61K 31/5377; A61K 31/538; A61K 47/10; A61K 47/12; C07D 401/04; C07D 405/14; C07D 413/14; C07D 417/14; C07D 471/04; C07D 487/04; C07D 491/107; C07D 498/04

(52) U.S. Cl. CPC C07D 401/14; A61K 9/0019; A61K 9/2013; A61K 9/2018; A61K 9/2027; A61K 9/2054; A61K 9/2059; A61K 9/2813; A61K 9/2866; A61K 9/4858; A61K 9/4866; A61K 31/4439; A61K 31/4545; A61K 31/496; A61K 31/497; A61K 31/501; A61K 31/506; A61K 31/53; A61K 31/5377; A61K 31/538; A61K 47/10; A61K 47/12; C07D 401/04; C07D 405/14; C07D 413/14; C07D 417/14; C07D 471/04; C07D 487/04; C07D 491/107; C07D 498/04

(57) ABSTRACT

The invention relates to a compound of formula (I)

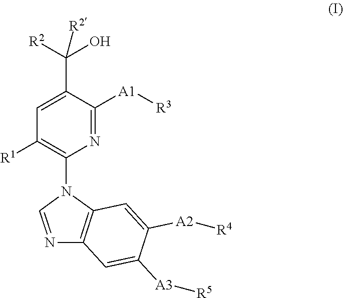

wherein A1, A2, A3, R¹, R², R^{2′}, R³, R⁴ and R⁵ are as defined in the description and in the claims. The compound of formula (I) can be used as a medicament.

### CROSS REFERENCE TO RELATED APPLICATIONS

This application is a continuation of International Application No. PCT/EP2023/051059, filed Jan. 18, 2023, which claims priority to European Application number 22152225.3 filed Jan. 19, 2022, which are incorporated herein by reference in their entirety.

### FIELD OF INVENTION

The present invention relates to organic compounds useful for therapy and/or prophylaxis in a mammal, and in particular to compounds that modulate SIK activity.

### BACKGROUND OF INVENTION

Salt-inducible kinases (SIK) belong to a subfamily of AMP-activated protein kinases (AMPK) called AMPK-related kinases. There are three members, named SIK1, SIK2 and SIK3, that are broadly expressed. Their major biological role is to modify gene expression by controlling the phosphorylation and subcellular localization of two key classes of transcriptional regulatory factors: CRTCs (cAMP-regulated transcriptional coactivators) and class IIa HDACs (Histone deacetylases). Indeed, in basal state, both CRTCs and HDACs are phosphorylated by SIK kinases, and sequestered in the cytoplasm through interactions with their cytoplasmic chaperones 14-3-3. In response to extracellular cues that usually increase intracellular levels of cAMP, the SIK kinases' activity is inhibited, CRTCs and HDACs are no longer phosphorylated and are hence released from 14-3-3. They can therefore translocate into the nucleus and regulate gene expression (reviewed in Wein et al., Trends Endocrinol Metab. 2018 October; 29(10):723-735).

In macrophages, the inhibition of SIK kinases leads to 1) CRTC3 shuttling to the nucleus and increasing the transcription of IL-10; and 2) translocation of HDAC4/5 to the nucleus and subsequent deacetylation of NF-κB resulting in decreased transcription of pro-inflammatory cytokines (Clark et al., Proc Natl Acad Sci USA. 2012 Oct. 16; 109(42):16986-91.).

Macrophages are critical to maintaining tissue homeostasis, mediating inflammation, and promoting the resolution of inflammation. To achieve this diversity of function, macrophages have the ability to “polarize” differently in response to environment cues. The two extreme phenotypes along their activation state continuum are the “M1” or “pro-inflammatory macrophages” and the “M2” or “pro-resolution macrophages”.

Strikingly, the inhibition of intracellular SIK kinases overrides these extracellular macrophage polarization signals and pushes them toward a pro-resolution phenotype. This comes with an increase in IL-10 (by interfering with the SIK-CRTC3 pathway) and a concomitant decrease in TNF-α, IL-12 and IL-6 (by interfering with the SIK-HDAC4/5 and NF-κB pathway). The high levels of IL-10 and low levels of pro-inflammatory cytokines upon SIK inhibition will promote resolution of inflammation. The exploration of the SIK pathway has initially been described in macrophages (Clark et al., Proc Natl Acad Sci USA. 2012 Oct. 16; 109(42):16986-91) and dendritic cells (Sundberg et al., Proc Natl Acad Sci USA. 2014 Aug. 26; 111(34):12468-73) and the therapeutic potential of pan-SIK inhibitors has been confirmed in a mouse LPS (lipopolysaccharide) challenge model (Sundberg et al., ACS Chem Biol. 2016 Aug. 19; 11(8):2105-11) and in colitis models (Fu et al., Inflamm Bowel Dis. 2021 Oct. 20; 27(11):1821-1831). SIKs have since been shown to be important players in the functions of several immune cells, including mast cells (Darling et al., J Biol Chem. 2021 January-June; 296:100428). Importantly, SIK1 is poorly expressed in macrophages and one embodiment of the invention are SIK2/3 inhibitors sparing SIK1, thus limiting potential SIK1-related toxicities.

SIK inhibitors have a high therapeutic potential in diseases that are 1) characterized by pro-inflammatory macrophage influx in the tissues and impaired tissue homeostasis and healing, or 2) where anti-TNF therapies are beneficial (partially or fully) or with insufficient levels of the IL10. Diseases with an inflammatory macrophage signature are e.g. rheumatoid arthritis, juvenile rheumatoid arthritis, NASH, primary sclerosing cholangitis, giant cell vasculitis and inflammatory bowel diseases (“IBD”), atherosclerosis, type 2 diabetes and glomerulonephritis.

Diseases with a proven link to IL-10 and TNF-α are IBD. Genetic alterations that reduce the function of IL-10 (such as SNPs in IL-10 or its receptor) are associated with an increased risk for IBD in humans. In addition, anti-TNF therapies are successful but only a subset of IBD patients are responsive and much of this limited responsiveness is lost over time. The described dual effect of SIK inhibitors (increased IL-10 and decreased TNF-α) make them particularly pertinent for the treatment of IBD.

All three SIK kinase isoforms are expressed broadly in human tissues with the highest expression observed in skin and adipose tissues for SIK1, adipose tissue for SIK2 and testis and brain for SIK3. Similarly to their role in macrophages, SIKs in these cells phosphorylate CRTCs and class II HDCAs in response to extracellular signals, which subsequently change the expression of several cellular factors.

In addition to their physiological roles, reports have linked dysregulation of SIK expression to a few diseases. For example, SIK2 has been described as a risk locus for primary sclerosing cholangitis, a fibrotic disease regularly associated with IBD. In addition, SIK2 and SIK3 expression is higher in ovarian and prostate cancers and correlated with poor survival (Miranda et al., Cancer Cell. 2016 Aug. 8; 30(2):273-289; Bon et al., Mol Cancer Res. 2015 April; 13(4):620-635).

As of today many diseases caused by dysregulation of the innate immune system lack efficient therapies and there is a high unmet medical need for new therapies. The present invention relates to a novel compounds that are highly active SIK inhibitors for the treatment of inflammatory, allergic and autoimmune diseases. In addition to inflammation, allergic and autoimmune diseases, SIK inhibitors can thus also be of potential relevance in cancer, metabolic diseases, bone density dysregulation diseases, pigmentation-related diseases or cosmetology, fibrotic diseases and depressive disorders.

### SUMMARY OF INVENTION

The invention relates in particular to a compound of formula (I)

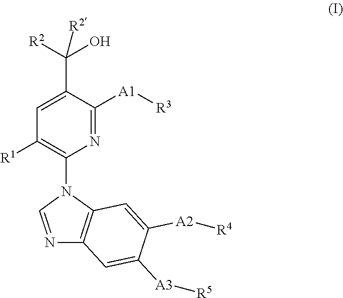

wherein
R¹ is hydrogen or halogen;
R² and R^{2′} are independently selected from hydrogen, alkyl, cyclopropyl, haloalkyl and alkoxyalkyl;
A1 is —O—, —NR⁶—, —(C═O)— or a bond;
R⁶ is hydrogen or alkyl;
R³ is hydroxyalkyl, heterocycloalkyl, heteroaryl, phenyl or cycloalkylalkyl, wherein heterocycloalkyl, heteroaryl, phenyl and cycloalkylalkyl are optionally substituted with 1, 2 or 3 substituents independently selected from R⁷; if A1 is —(C═O)—, then R³ can also be haloalkylamino;
each R⁷ is independently selected from alkoxy, alkylamino, alkyl, aminocarbonyl, amino, cyano, cycloalkylamino, haloalkyl, halocycloalkyl, halogen, heteroaryl, hydroxycarbonylamino, alkoxyalkyl, alkylaminocarbonyl, alkylsulfonyl, alkoxycarbonimidoyl, aminocarbonyl, hydroxy, cycloalkylalkyl, haloalkoxy, heterocycloalkyl and cycloalkyl;
A2 is —O—, —NH— or a bond;
R⁴ is hydrogen, alkyl, haloalkyl, alkoxyalkyl, dialkylaminoalkyl, cycloalkyl, cycloalkylcarbonyl, aryl, heteroaryl, heteroarylalkyl, heterocycloalkyl or heterocycloalkylalkyl; wherein aryl, cycloalkyl, heteroaryl, heteroarylalkyl, heterocycloalkyl and heterocycloalkylalkyl are optionally substituted with 1, 2 or 3 substituents independently selected from R⁸; if A2 is a bond, then R⁴ can also be halogen or cyano;
each R⁸ is independently selected from alkyl, halogen, cyano, alkylsulfonyl, alkylaminocarbonyl, heterocycloalkyl and alkoxyheterocycloalkylalkyl;
A3 is —O—, —NR¹⁰— or a bond;
R⁵ is hydrogen, alkyl, alkylsulfonyl, cycloalkylcarbonyl, heterocycloalkylalkyl, heterocycloalkylalkylcarbonyl, aryl, heteroaryl or heterocycloalkyl, wherein cycloalkylcarbonyl, aryl, heteroaryl and heterocycloalkyl are optionally substituted with 1, 2 or 3 substituents independently selected from R⁹; if A3 is a bond, then R⁵ can also be halogen or cyano;
each R⁹ is independently selected from alkoxy, halogen, dialkylaminocarbonyl, alkyl, alkoxyalkoxy, alkoxyheterocycloalkylalkyl, alkoxyheterocycloalkylcarbonyl, haloalkyl, haloalkoxy, heterocycloalkylalkoxy, heterocycloalkyl, heterocycloalkyloxy, hydroxy, hydroxyalkyl, alkylheterocycloalkyl, (haloalkyl)cycloalkyl, alkylheterocycloalkylalkyl, heterocycloalkylalkyl, alkylsulfonyl, (alkyl)heterocycloalkyl, alkylheterocycloalkyloxy, heterocycloalkylheterocycloalkyl, heterocycloalkylheterocycloalkyl, CH₃—O—(CH₂—CH₂—O)ₙ—, alkylaminocarbonyl and cyano; wherein n is selected from 5, 6, 7, 8 and 9; and
R¹⁰ is hydrogen or alkylcarbonyl;
or a pharmaceutically acceptable salt thereof.

### DETAILED DESCRIPTION OF INVENTION

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C1-C8 alkyl groups include, but are not limited to, methyl, ethyl, propyl, isopropyl, butyl, isobutyl, tert.-butyl, the isomeric pentyls, the isomeric hexyls, the isomeric heptyls and the isomeric octyls, particularly methyl, ethyl, propyl, butyl and pentyl. Particular examples of alkyl are methyl, ethyl, propyl, isopropyl, butyl and isobutyl. Methyl, ethyl, propyl and butyl, like isobutyl, are further particular examples of “alkyl” in the compound of formula (I).

The term “cycloalkyl”, alone or in combination, signifies a cycloalkyl ring with 3 to 8 carbon atoms and particularly a cycloalkyl ring with 3 to 6 carbon atoms. Examples of cycloalkyl include, but are not limited to, cyclopropyl, cyclobutyl, cyclopentyl, cyclohexyl, cycloheptyl and cyclooctyl. Particular examples of “cycloalkyl” are cyclopropyl and cyclobutyl.

The term “heterocycloalkyl”, alone or in combination, denotes a monovalent saturated or partly unsaturated mono- or bicyclic ring system of 4 to 12 ring atoms, comprising 1, 2, or 3 ring heteroatoms selected from N, O and S, the remaining ring atoms being carbon. Bicyclic means consisting of two cycles having one or two ring atoms in common. “Hetercycloylkyl” may comprise a carbonyl group, wherein the carbon is part of the ring system. The ring system can be attached to the remaining compound via an atom selected from C, N, S and O, in particular via a N atom (“N-heterocycloalkyl).

Examples of “heterocycloalkyl” include, but are not limited to, morpholino, morpholin-4-yl, pyrrolidinyl, pyrrolidin-1-yl, pyrrolidin-3-yl, piperidinyl, 1-piperidyl, 4-piperidyl, 2-oxopyrrolidin-1-yl, piperazinyl, piperazin-1-yl, azetidinyl, azetidin-1-yl, [3-oxo-piperazin-1-yl], (1,1-dioxo-1,2-thiazolidin-2-yl), (4,5,6,7-tetrahydropyrazolo[4,3-c]pyridin-1-yl), (3-oxo-1,5,6,8-tetrahydrooxazolo[3,4-a]pyrazin-7-yl), [rac-(3aR,6aS)-2,3,3a,5,6,6a-hexahydro-1H-pyrrolo[3,2-b]pyrrol-4-yl], [rac-(3aS,6aR)-2,3,3a,5,6,6a-hexahydro-1H-pyrrolo[3,2-b]pyrrol-4-yl], (4-oxo-6,7-dihydro-5H-pyrazolo[1,5-a]pyrazin-3-yl), (6,7-dihydro-4H-pyrazolo[4,3-c]pyridin-1-yl), (4,7-diazaspiro[2.5]octan-7-yl), (2-oxa-5,8-diazaspiro[3.5]nonan-8-yl), 3-azabicyclo[3.2.0]heptan-3-yl), (5-azaspiro[2.4]heptan-5-yl), (2-azabicyclo[2.2.1]heptan-2-yl), 4-oxa-7-azaspiro[2.5]octan-7-yl, (3-azabicyclo[3.1.0]hexan-3-yl), (6,7-dihydro-4H-pyrazolo[4,3-c]pyridin-1-yl), 2-oxa-7-azaspiro[3.4]octan-7-yl, (2-oxo-1-piperidyl), (2,3-dihydropyridazino[4,5-b][1,4]oxazin-8-yl), pyrrolidin-1-yl, 2-oxo-pyrimidin-4-yl, morpholinoethyl, 2-oxa-5-azaspiro[3.4]octan-5-yl, oxetan-3-yl, (2-oxo-1-piperidyl), 2-oxo-4-piperidyl, 5-oxo-pyrrolidin-3-yl, 2-oxa-5-azaspiro[3.4]octan-5-yl, (7,8-dihydro-5H-pyrano[4,3-c]pyridazin-3-yl), [rac-(4aS,7aR)-4-methyl-2,3,4a,5,7,7a-hexahydropyrrolo[3,4-b][1,4]oxazin-6-yl], 6,7-dihydro-5H-cyclopenta[c]pyridazin-3-yl, 5,6-dihydropyrrolo[2,3-c]pyridazin-7-yl, 7-oxo-5H-pyrrolo[3,4-b]pyridin-2-yl, 5,6-dihydro-4H-pyrazolo[4,3-c]pyridin-1-yl, 6-azaspiro[3.4]octan-6-yl and [rac-(3aS,6aS)-6-oxo-2,3,3a,4,5,6a-hexahydropyrrolo[2,3-c]pyrrol-1-yl]. Particular examples of “heterocycloalkyl” is pyrrolidin-1-yl and pyrrolidin-3-yl. In one particular embodiment, heterocycloalkyl is “N-heterocycloalkyl”.

The term “heteroaryl”, alone or in combination, signifies an aromatic mono- or bicyclic ring system with 5 to 12 ring atoms, comprising 1, 2, 3 or 4 heteroatoms each independently selected from N, O and S, the remaining ring atoms being carbon. The ring system can be attached to the remaining compound via an atom selected from C, N, S and O, in particular via a N atom (“N-heteroaryl).

Examples of heteroaryl include, but are not limited to, pyrazolyl, pyrazol-1-yl, pyrazol-3-yl, pyrazol-4-yl, pyridinyl, 2-pyridyl, 3-pyridyl, 4-pyridyl, pyridazinyl, pyridazin-3-yl, pyridazin-4-yl, pyrazinyl, pyrazin-2-yl, isoxazolyl, isoxazol-3-yl, isoxazol-4-yl, oxazolyl, 2-oxazol-5-yl, 2-oxo-3-imidazol-1-yl, pyrimidinyl, pyrimidin-5-yl, benzotriazolyl, 1H-benzotriazol-4-yl, furanyl, furyl, 2-furyl, 3-furyl, [6-oxo-1H-pyridazin-5-yl], triazolyl, triazol-1-yl, triazol-2-yl, triazol-3-yl, 2-oxo-4-pyridyl, pyrimidin-2-yl, pyrimidin-5-yl, (1,3,4-oxadiazol-2-yl), (1,3,4-thiadiazol-2-yl), (1,2,4-triazin-3-yl), 2-oxo-pyrimidin-4-yl, (1-methyl-2-oxo-3-pyridyl) and (2,3-dihydropyridazino[4,5-b][1,4]oxazin-8-yl). Particular examples of “heteroaryl” are pyrazol-1-yl, pyrazol-4-yl, pyridazin-3-yl ane pyrimidin-5-yl. In one particular embodiment, heteroaryl is “N-heteroaryl”.

The term “aryl” denotes a monovalent aromatic carbocyclic mono- or bicyclic ring system comprising 6 to 10 carbon ring atoms. Examples of aryl moieties include phenyl and naphthyl. A particular examples of aryl is phenyl.

The term “alkoxy” or “alkyloxy”, alone or in combination, signifies a group of the formula alkyl-O— in which the term “alkyl” has the previously given significance, such as methoxy, ethoxy, n-propoxy, isopropoxy, n-butoxy, isobutoxy, sec-butoxy and tert.-butoxy. Particular examples of “alkoxy” are methoxy and ethoxy.

The term “oxy”, alone or in combination, signifies the —O— group.

The term “oxo”, alone or in combination, signifies the ═O group.

The terms “halogen” or “halo”, alone or in combination, signifies fluorine, chlorine, bromine or iodine and particularly fluorine, chlorine or bromine, more particularly fluorine. The term “halo”, in combination with another group, denotes the substitution of said group with at least one halogen, particularly substituted with one to five halogens, particularly one to four halogens, i.e. one, two, three or four halogens.

The term “haloalkyl”, alone or in combination, denotes an alkyl group substituted with at least one halogen, particularly substituted with one to five halogens, particularly one to three halogens, more particularly two to three halogens. Particular “haloalkyl” are fluoromethyl, fluoroethyl, fluoropropyl, fluorobutyl, difluoromethyl, difluoroethyl, trifluoromethyl and trifluoroethyl.

The term “haloalkoxy”, alone or in combination, denotes an alkoxy group substituted with at least one halogen, particularly substituted with one to five halogens, particularly one to three halogens.

Particular “haloalkoxy” are fluoromethoxy, fluoroethoxy and fluoropropyloxy.

The terms “hydroxyl” and “hydroxy”, alone or in combination, signify the —OH group.

The term “carbonyl”, alone or in combination, signifies the —C(O)— group.

The term “amino”, alone or in combination, signifies the primary amino group (—NH₂), the secondary amino group (—NH—), or the tertiary amino group (—N—).

The term “alkylamino” is alkyl group linked to a —NH— group. The term “dialkylamino” denotes two alkyl groups linked to a —N— atom.

The term “sulfonyl”, alone or in combination, signifies the —SO₂— group.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particular pharmaceutically acceptable salts of compounds of formula (I) are the salts of trifluoroacetic acid, formic acid, hydrochloric acid, hydrobromic acid, sulfuric acid, phosphoric acid and methanesulfonic acid.

Tautomeric forms, i.e. structural isomers which interconvert with the compound of formula (I), in particular in solution, exist.

For instance, the compound of formula (I) wherein A3 is —NH— and R5 is pyridazin-3-yl, is in tautomeric equilibrium with its tautomeric form (I′):

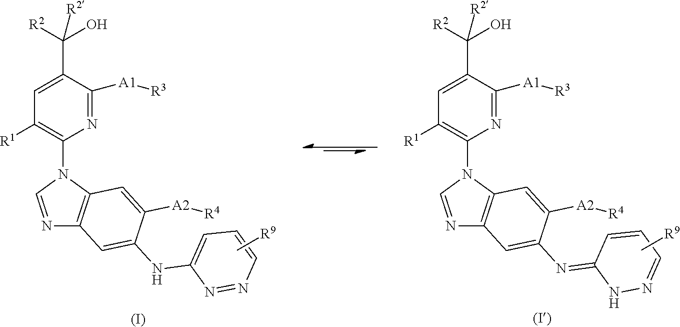

Other tautomeric forms of the compound of formula (I) may also exist.

The compound of formula (I) can be represented by different mesomeric structures such as for instance:

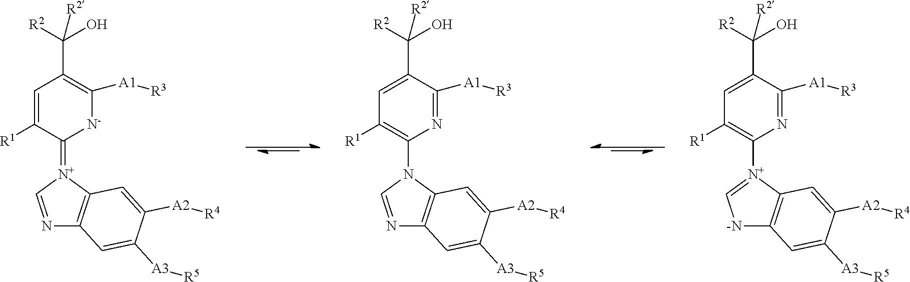

If one of the starting materials or compounds of formula (I) contain one or more functional groups which are not stable or are reactive under the reaction conditions of one or more reaction steps, appropriate protecting groups (as described e.g. in “Protective Groups in Organic Chemistry” by T. W.

Greene and P. G. M. Wuts, 3^{rd} Ed., 1999, Wiley, New York) can be introduced before the critical step applying methods well known in the art. Such protecting groups can be removed at a later stage of the synthesis using standard methods described in the literature. Examples of protecting groups are tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz).

The compound of formula (I) can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates.

The term “asymmetric carbon atom” means a carbon atom with four different substituents. According to the Cahn-Ingold-Prelog Convention an asymmetric carbon atom can be of the “R” or “S” configuration.

Furthermore, the invention includes all optical isomers, i.e. diastereoisomers, diastereomeric mixtures, racemic mixtures, all their corresponding enantiomers and/or tautomers as well as their solvates, wherever applicable, of the compound of formula (I).

If desired, racemic mixtures of the compound of the invention may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography.

In the embodiments, where an optically pure enantiomer is provided, optically pure enantiomer means that the compound contains >90% of the desired isomer by weight, particularly >95% of the desired isomer by weight, or more particularly >99% of the desired isomer by weight, said weight percent based upon the total weight of the isomer of the compound. A chirally pure or chirally enriched compound may be prepared by chirally selective synthesis or by separation of enantiomers. The separation of enantiomers may be carried out on the final product or alternatively on a suitable intermediate.

Furthermore, the invention includes all substituents in their corresponding deuterated form, wherever applicable, of the compound of formula (I).

Furthermore, the invention includes all substituents in their corresponding tritiated form, wherever applicable, of the compound of formula (I).

A certain embodiment of the invention relates to the compound of formula (I) as described herein, or a pharmaceutically acceptable salt thereof, wherein at least one substituent comprises at least one radioisotope. Particular examples of radioisotopes are 2H, 3H, 13C, 14C and 18F.

### General Synthetic Schemes

The synthesis of the compound of formula (I) can, for example, be accomplished according to the non-exhaustive procedures described below in general schemes 1-11. In some instances, the sequence of the reaction steps can be altered and the individual steps of the different schemes can be combined in different ways as disclosed herein and according to common general knowledge. In general, the reaction conditions provided below and the reaction conditions can in some instances be further modified according to the procedures described herein and according to common general knowledge.

### Scheme 1

In scheme 1, the synthesis of a compound of formula (I-a) is described. The compound of formula (I-a) is a compound of formula (I), wherein each of A1 is a bond; A2 is —O—; A3 is —O—; R¹ is hydrogen; R² and R^{2′} are both hydrogen; R³ is phenyl optionally substituted with one, two or three substituents independently selected from R⁷; R⁴ is alky; R⁵ is alky; each R⁷ is independently selected from alkoxy and halogen.

Error! Objects cannot be created from editing field codes.

Step A: Methyl 2,6-dichloronicotinate 1 can be reacted with a 5,6-disubstituted benzimidazole 2 in the presence of a strong base such as for instance NaH in a polar solvent such as DMF or DMSO at about 0° C. to yield intermediate 3.

Step B: Intermediate 3 can undergo a palladium-catalyzed cross-coupling reaction (Suzuki-Miyaura) with a suitable reactant such as for instance the corresponding aryl boronic acid or aryl pinacol borane, catalytic Pd(PPh₃)₄ or other suitable Pd catalysts in presence of a suitable base such as for instance Na₂CO₃, and in a suitable solvent such as for instance DME while heating (e.g. MW microwave at 120° C.) to yield intermediate 4.

Step C: The ester of 4 can be reduced to the corresponding alcohol using a suitable reducing agent such as for instance LiAlH₄ in a suitable solvent such as for instance THF at 0° C. to yield the compound of formula (I-a).

### Scheme 2

In scheme 2, the synthesis of a racemic compound of formula (I-b), as well as the corresponding entantiomers (Compounds I-b′ and I-b″) is described. The compound of formula (I-b) is a compound of formula (I), wherein each of A1 is a bond; A2 is —O—; A3 is —O—; R¹ is hydrogen; R² is alkyl; R^{2′} is hydrogen; R³ is phenyl optionally substituted with one, two or three substituents independently selected from R⁷; R⁴ is alky; R⁵ is alky; each R⁷ is independently selected from alkoxy and halogen.
Error! Objects cannot be created from editing field codes.

Step A: In the presence of a suitable oxidation reagent such as for instance DMP (Dess-Martin periodinane) in a suitable solvent such as for instance DCM, the alcohol group of Compound (I-a) in Scheme 1 can be oxidized to the corresponding aldehyde 5 at ambient temperature.

Step B: The aldehyde 5 can be reacted with a suitable alkyl magnesium halide (such as for instance MeMgBr or EtMgBr or cyclopropylMgBr) at −30° C. to 0° C. in a suitable solvent such as for instance a mixture of THF/2-MeTHF to yield the corresponding secondary alcohol I-b.

Step C: The racemic mixture of the secondary alcohol I-b can be separated by chiral SFC into the enantiomers I-b′ and I-b″.

### Scheme 3

In scheme 3, the synthesis of a compound of formula (I-c) is described. The compound of formula (I-c) is a compound of formula (I), wherein each of A1 is a bond; A2 is —O—; A3 is —O—; R¹ is hydrogen; R² is hydrogen; R^{2′} is hydrogen; R³ is N-heterocycloalkyl; R⁴ is alky; R⁵ is alky.

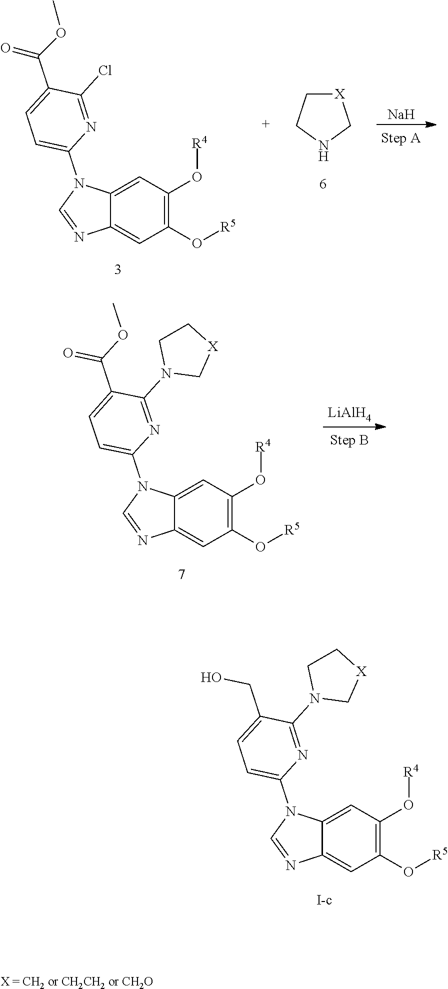

Step A: Intermediate 3 from Scheme 1 can be substituted with a saturated N-heterocycle 6 in the presence of a strong base (such as for instance NaH, or Cs₂CO₃ or other carbonates) in a polar solvent (such as for instance DMF, DMA, NMP or DMSO) to yield intermediate 7.

Step B: The ester of the intermediate 7 can be reduced in the presence of a reducing agent, such as for instance LiAlH₄, to the a compound of formula (I-c), in a similar manner than shown step C in scheme 1.

### Scheme 4

In scheme 4, the synthesis of a compound of formula (J-d) and a compound of formula (I-e) is described. The compound of formula (J-d) is a compound of formula (I), wherein A1 is a bond; A2 is —O—; A3 is —O—; R¹ is hydrogen; R² is hydrogen; R^{2′} is hydrogen; R³ is N-heterocycloalkyl; R4 is alkyl; R5 is alkyl. The compound of formula (I-e) is a compound of formula (I), wherein A1 is —NH—; A2 is —O—; A3 is —O—; R¹ is hydrogen; R² is hydrogen; R^{2′} is hydrogen; R³ is hydroxyalkyl; R⁴ is alkyl; R⁵ is alkyl.

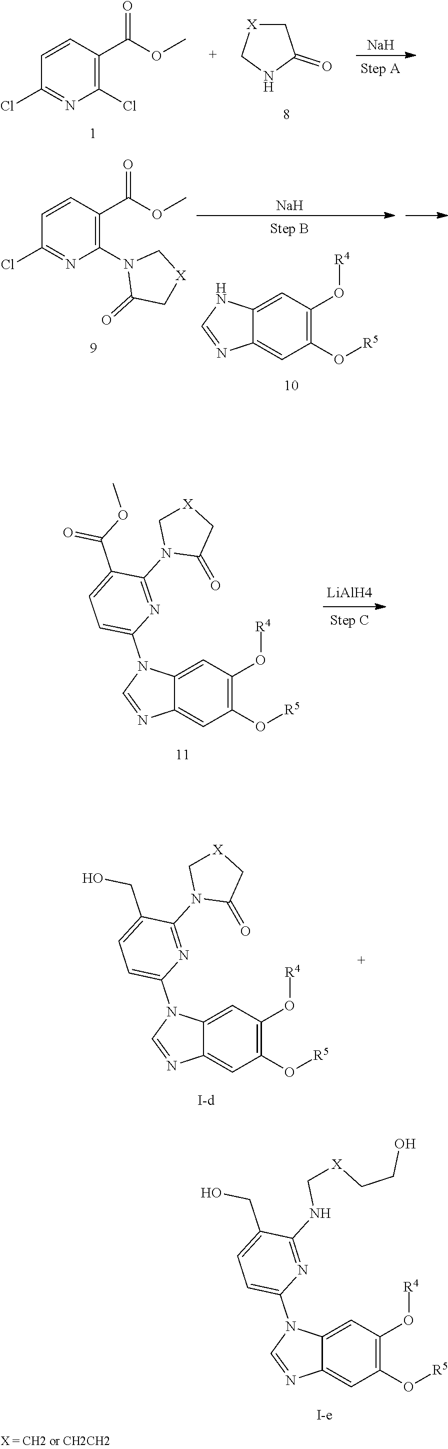

Step A: Methyl 2,6-dichloronicotinate 1 can be reacted with a cyclic amide 8 in the presence of a suitable base such as for instance NaH in a suitable solvent such as DMF at −10° C.-0° C. to yield intermediate 9.

Step B: Further substitution of intermediate 9 with a 5,6-disubstituted benzimidazole in the presence of a strong base such for instance NaH in a polar solvent (such as for instance DMF, DMA, NMP or DMSO) at 0° C. yields intermediate 11.

Step C: The ester of intermediate 11 can be reduced in the presence of a suitable reducing agent such as for instance LiAlH₄ to yield the compound of formula (I-d), in a similar manner than in step C in scheme 1. The open chain compound (I-e) can also be obtained via the reduction step.

### Scheme 5

In scheme 5, the synthesis of a racemic compound of formula (I-f), as well as the corresponding entantiomers (Compounds I-f′ and I-f″) is described. The compound of formula (I-f) is a compound of formula (I), wherein each of A1 is a bond; A2 is —O—; A3 is —O—; R¹ is hydrogen; R² is alkyl; R^{2′} is hydrogen; R³ is phenyl or heteroaryl, wherein phenyl and heteroaryl are optionally substituted with one, two or three substituents independently selected from R7; R4 is alky; R⁵ is alky; each R⁷ is independently selected from aminocarbonyl, methyl and cyano.

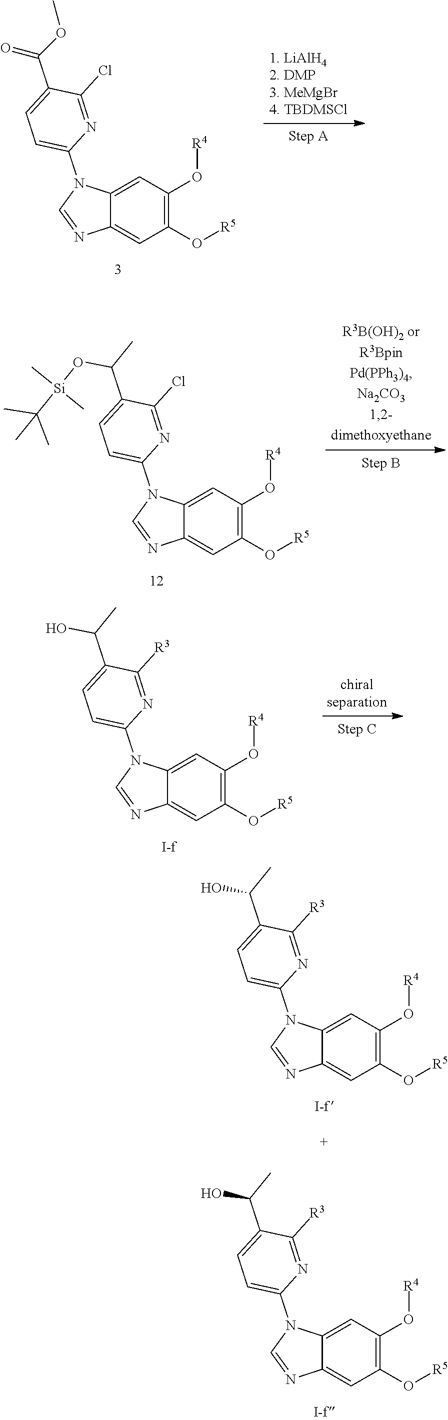

Step A: Intermediate 12 can be obtained from Intermediate 3 from Scheme 1 through a sequence of (1) reduction with a suitable reducing agent such as for instance LiAlH4 in a suitable solvent such as for instance THF at 0° C., (2) oxidation with suitable reagent such as for instance DMP at room temperature to yield an aldehyde, (3) reaction with a suitable alkyl magnesium halide (such as for instance MeMgBr or EtMgBr or cyclopropylMgBr) in THF at −20° C.-0° C., and finally protection of the corresponding secondary alcohol with TBDMSCl in DCM in the presence of imidazole.

Step B: A Palladium-catalyzed cross-coupling reaction (Suzuki-Miyaura) with the corresponding aryl boronic acid or aryl pinacol borane, catalytic Pd(PPh₃)₄ or the suitable Pd catalysts and a suitable base (K₃PO₄, Cs₂CO₃, K₂CO₃, Na₂CO₃) in a suitable solvent (DME) while heating (e.g. MW at 80° C.-120° C.) yields a compound of formula (I-f).

Step C: The racemic mixture of the secondary alcohol I-f can be further separated by chiral SFC into the enantiomers I-f and I-f″.

### Scheme 6

In scheme 6, the synthesis of a racemic compound of formula (J-g), as well as the corresponding entantiomers (Compounds I-g′ and I-g″) is described. In scheme 6, the synthesis of a racemic compound of formula (I-h), as well as the corresponding entantiomers (Compounds I-h′ and I-h″) is also described.

The compound of formula (I-g) is a compound of formula (I), wherein A1 is a bond; A2 is a bond; A3 is —NH—: R¹ is hydrogen; R² is alkyl; R^{2′} is hydrogen; R³ is pyrazol-1-yl optionally substituted with one, two or three substituents independently selected from R⁷; R⁴ is hydrogen; R¹ is pyridazin-3-yl optionally substituted with R⁹; each R⁷ is independently selected from alkyl, cyano, haloalkyl, alkoxy, alkylaminocarbonyl and alkylsulfonyl; R⁹ is alkyl. The compound of formula (I-h) is a compound of formula (I), wherein A1 is a bond; A2 is a —NH—; A3 is a bond: R¹ is hydrogen; R² is alkyl; R^{2′} is hydrogen; R³ is pyrazol-1-yl optionally substituted with one, two or three substituents independently selected from R⁷; R⁴ is pyridazin-3-yl optionally substituted with R⁸; R¹ is hydrogen; each R⁷ is independently selected from alkyl, cyano, haloalkyl, alkoxy, alkylaminocarbonyl and alkylsulfonyl; R⁸ is alkyl.

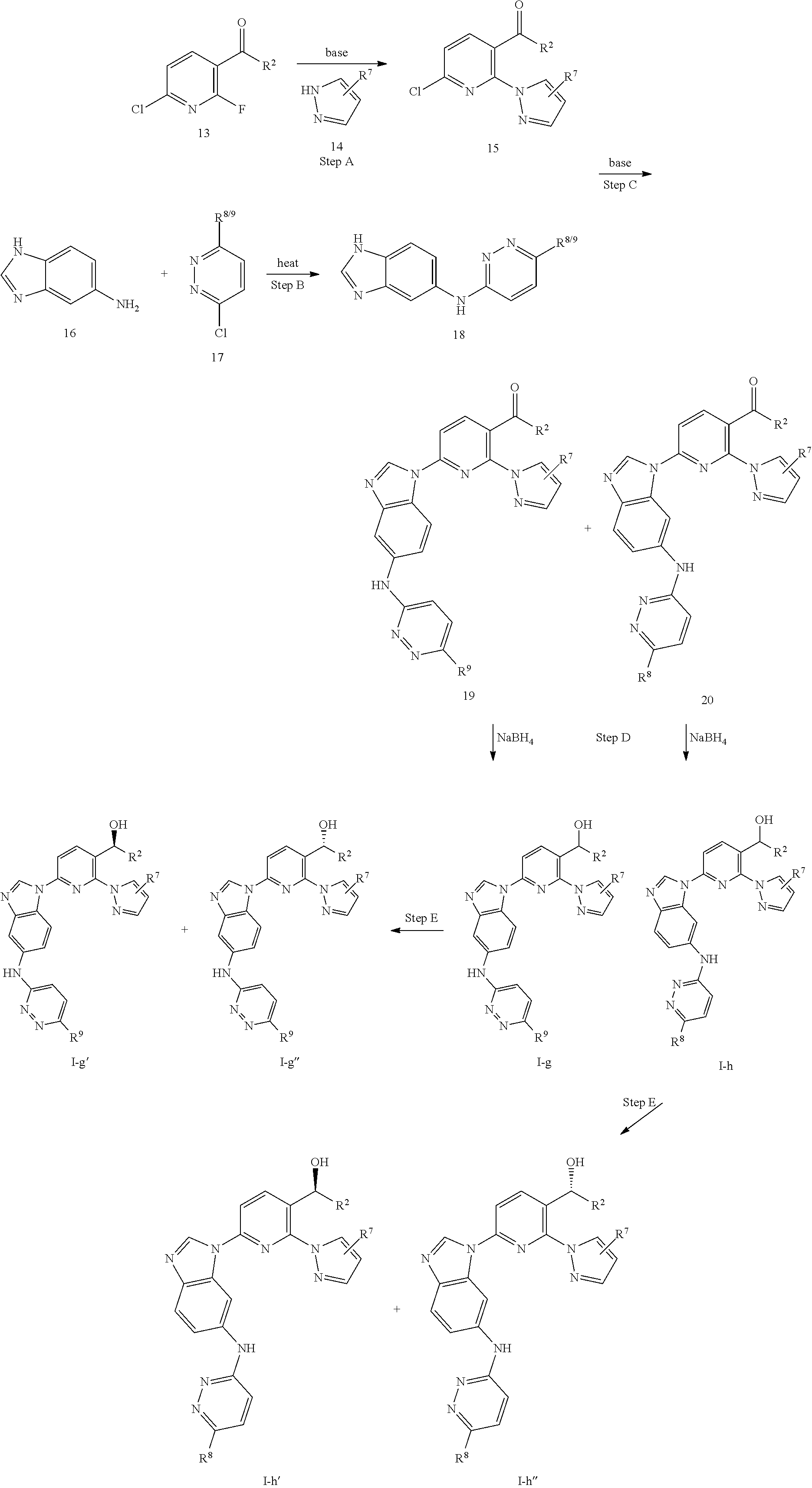

Step A: 1-(6-chloro-2-fluoro-3-pyridyl)alkanone 13 can be reacted with a substituted pyrazole 14 in the presence of a suitable organic or mineral base, such as for instance DIPEA, DBU, K₂CO₃, Cs₂CO₃ or NaH, in a suitable polar solvent (for instance DMF, DMA, NMP, DMSO or THF, MeTHF) to yield intermediate 15.

Step B: Intermediate 18 can be obtained from the reaction of 5-aminobenzimidazole 16 and (6-alkylpyridazin-3-yl)amine 17 in iPrOH while heating to reflux.

Step C: The intermediates 15 and 18 can be combined in the presence of a suitable organic or mineral base (such as for instance DIPEA, DBU, K₂CO₃, Cs₂CO₃ or NaH) in a suitable polar solvent (such as for instance DMF, DMA, NMP, DMSO or THF, MeTHF) to yield the regioisomeric intermediates 19 and 20 which can be separated by flash column chromatography.

Step D: The corresponding secondary alcohols (I-g and I-h) can be obtained after reduction of the carbonyl group with a suitable reducing agent such as for instance NaBH₄ or NaCNBH₃ (1-5 equiv.) in a suitable solvent such as for instance MeOH (and in some cases a second co-solvent, e.g. DCM, THF, DMF) at temperatures between −40° C. and rt.

Step E: The racemic mixture of the secondary alcohol I-g can be further separated by chiral SFC into the enantiomers J-g′ and J-g″. Similarly, the racemic mixture of the secondary alcohol I-h can be further separated by chiral SFC into the enantiomers I-h′ and I-h″.

### Scheme 7

In scheme 7, the synthesis of a compound of formula (I-i) and a compound of formula (I-j) is described. The compound of formula (I-i) is a compound of formula (I), wherein A1 is a bond; A2 is a bond; A3 is —NH—: R¹ is hydrogen; R² is alkyl; R^{2′} is hydrogen; R³ is pyrazol-1-yl optionally substituted with one, two or three substituents independently selected from R⁷; R⁴ is hydrogen; R⁵ is alkyl, heterocycloalkyl or heteroaryl, wherein heterocycloalkyl and heteroaryl are optionally substituted with one, two or three substituents independently selected from R⁹; each R⁷ is independently selected from alkyl, cyano, haloalkyl, alkoxy, alkylaminocarbonyl and alkylsulfonyl; R⁹ is alkyl, cyano or alkoxy. The compound of formula (I-j) is a compound of formula (I), wherein A1 is a bond; A2 is —NH—; A3 is a bond: R¹ is hydrogen; R² is alkyl; R^{2′} is hydrogen; R³ is pyrazol-1-yl optionally substituted with one, two or three substituents independently selected from R⁷; R⁴ is alkyl, heterocycloalkyl or heteroaryl, wherein heterocycloalkyl and heteroaryl are optionally substituted with one, two or three substituents independently selected from R¹; R⁵ is hydrogen; each R⁷ is independently selected from alkyl, cyano, haloalkyl, alkoxy, alkylaminocarbonyl and alkylsulfonyl; R¹ is alkyl, cyano or alkoxy.

Error! Objects cannot be created from editing field codes.

Step A: The regioisomeric intermediates 22 and 23 can be obtained—similarly to the description of step C in scheme 6—using intermediate 15 and 5-bromobenzimidazole 21 as the second reagent.

Step B: Introduction of the R7-substituted amine can be performed via a Buchwald-Hartwig coupling using a suitable base such as for instance Cs₂CO₃ K₂CO₃ or K₃PO₄, and as suitable palladium catalyst such as for instance t-Buxphos-Pd-G3 or [tBuBrettPhos Pd(allyl)]OTf at between around 80° C. to around 90° C. to yield intermediates 24 and 25, respectively.

Step C: The corresponding secondary alcohols can be obtained after reduction of the carbonyl of intermediates 24 and 25 with a suitable reducing agent such as for instance NaBH₄ (1-5 equiv.) in a suitable solvent such as for instance MeOH (and in some cases a second co-solvent, e.g. DCM, THF or DMF) at temperatures between −40° C. and rt to yield a compound of formula (I-i) and a compound of formula (I-j), respectively. The enantiomers of the secondary alcohol can be further separated by chiral SFC.

### Scheme 8

In scheme 8, the synthesis of a compound of formula (I-k) and a compound of formula (1-1) is described. The compound of formula (I-k) is a compound of formula (I), wherein A1 is a bond; A2 is a bond; A3 is —NH—: R¹ is hydrogen; R² is alkyl; R^{2′} is hydrogen; R³ is pyrazol-1-yl optionally substituted with one, two or three substituents independently selected from R⁷; R⁴ is hydrogen; R¹ heteroaryl optionally substituted with one, two or three substituents independently selected from R⁹; each R⁷ is independently selected from alkyl, cyano, haloalkyl, alkoxy, alkylaminocarbonyl and alkylsulfonyl; R⁹ is alkyl, cyano or alkoxy. The compound of formula (I-j) is a compound of formula (I), wherein A1 is a bond; A2 is —NH—; A3 is a bond: R¹ is hydrogen; R² is alkyl; R^{2′} is hydrogen; R³ is pyrazol-1-yl optionally substituted with one, two or three substituents independently selected from R⁷; R⁴ is heteroaryl optionally substituted with one, two or three substituents independently selected from R⁸; R⁵ is hydrogen; each R⁷ is independently selected from alkyl, cyano, haloalkyl, alkoxy, alkylaminocarbonyl and alkylsulfonyl; R¹ is alkyl, cyano or alkoxy.

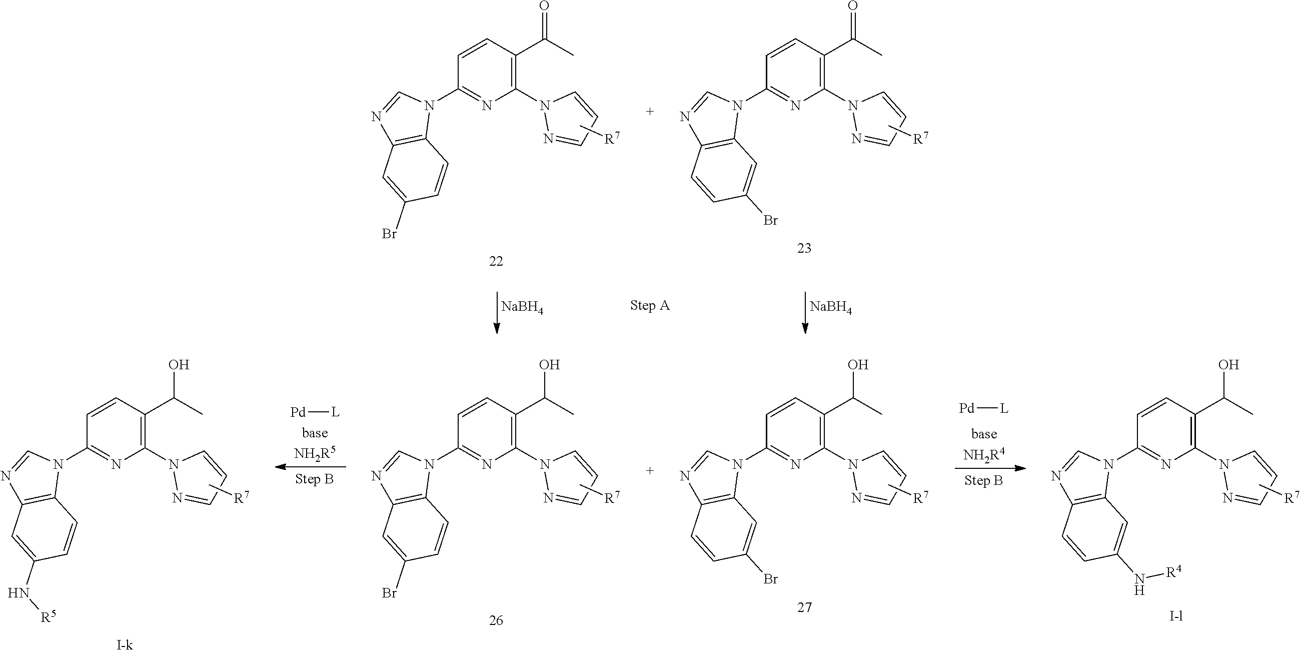

Step A: The carbonyl group of the regioisomer intermediates 22 and 23 obtained in step A of scheme 7 can be reduced with a suitable reducing agent such as for instance NaBH₄ or NaCNBH₃ in a suitable solvent such as for instance MeOH/THF (1:1) at around 0° C., to yield intermediates 26 and 27 respectively.

Step B: An arylamine group can be introduced via a Buchwald-Hartwig coupling using for instance Cs₂CO₃ as a base and RuPhos-Pd-G3 as catalyst in a suitable solvent such as for instance 1,4-dioxane at 100° C., to yield a compound of formula (I-k) and a compound of formula (I-1) respectively.

### Scheme 9

In scheme 9, the synthesis of a compound of formula (I-m) is described. The compound of formula (I-m) is a compound of formula (I), wherein A1 is a bond; A2 is a bond; A3 is —NH—: R¹ is hydrogen; R² is methyl; R^{2′} is hydrogen; R³ is N-heteroaryl optionally substituted with one, two or three substituents independently selected from R⁷; R⁴ is hydrogen; R¹ is pyridazin-3-yl substituted with methyl; each R⁷ is independently selected from alkyl, cyano, halogen, haloalkyl, haloalkoxy, hydroxy, alkoxy, and cycloalkylamino.

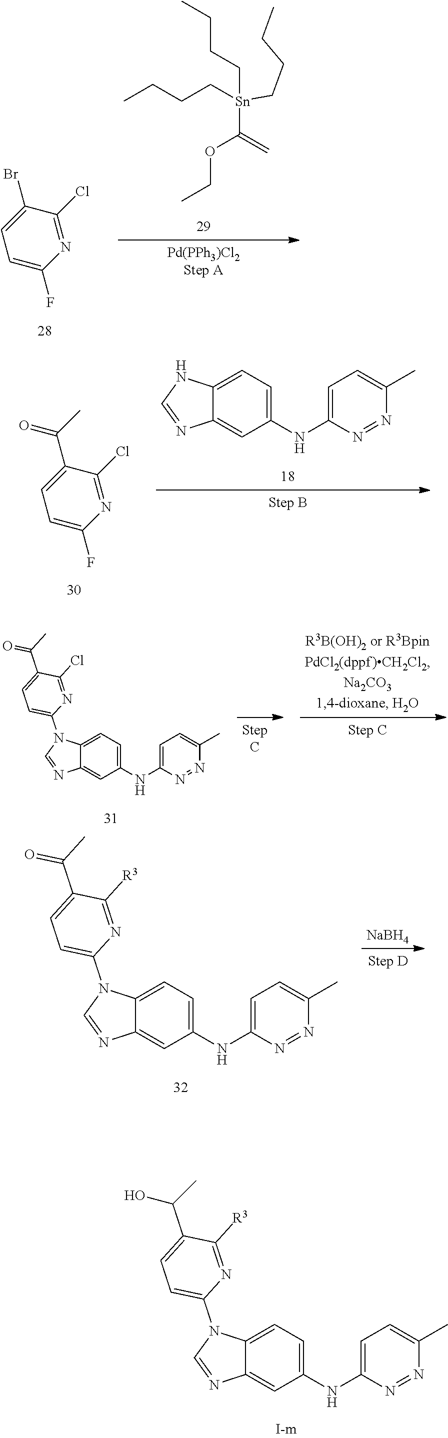

Step A: 1-(2-chloro-6-fluoro-3-pyridyl)ethanone 30 can be obtained from the reaction of 3-bromo-2-chloro-6-fluoro-pyridine 28 and tributyl(1-ethoxyvinyl)tin 29 in the presence of a suitable catalyst such as for instance Pd(PPh₃)Cl₂ and a suitable base such as for instance K₂CO₃, Cs₂CO₃ or K₃PO₄ in a suitable solvent such as for instance 1,4-dioxane and H₂O at around 100° C.

Step B: The intermediate 30 can then be reacted with N-(6-methylpyridazin-3-yl)-1H-benzimidazol-5-amine (intermediate 18 from Scheme 6) in DMSO at 60° C. to obtain intermediate 31 (and its regioisomer).

Step C: A palladium-catalyzed cross-coupling reaction (Suzuki-Miyaura) with the corresponding aryl boronic acid or aryl pinacol borane, a suitable catalyst such as for instance PdCl₂(dppf) CH₂Cl₂ and a suitable base (for instance Na₂CO₃) in a solvent such as for instance 1,4-dioxane while heating to around 100° C., yields intermediate 32.

Step D: The compound I-m can be obtained after reduction of the carbonyl group of 32 with a suitable reducing agent such as for instance NaBH₄ or NaCNBH₃ (1-5 equiv.) in a suitable solvent such as for instance MeOH (and in some cases a second co-solvent, e.g. DCM, THF or DMF) at temperatures between around −40° C. and rt.

### Scheme 10

In scheme 10, the synthesis of a compound of formula (I-n) is described. The compound of formula (I-n) is a compound of formula (I), wherein A1 is a bond; A2 is a bond; A3 is —NH—; R¹ is hydrogen; R² is methyl; R^{2′} is hydrogen; R³ is cycloalkyl optionally substituted with one, two or three substituents independently selected from R⁷; R⁴ is hydrogen; R¹ is pyridazin-3-yl substituted with methyl; each R⁷ is independently selected from alkyl, cyano, halogen, haloalkyl, haloalkoxy, hydroxy, alkoxy, and cycloalkylamino.

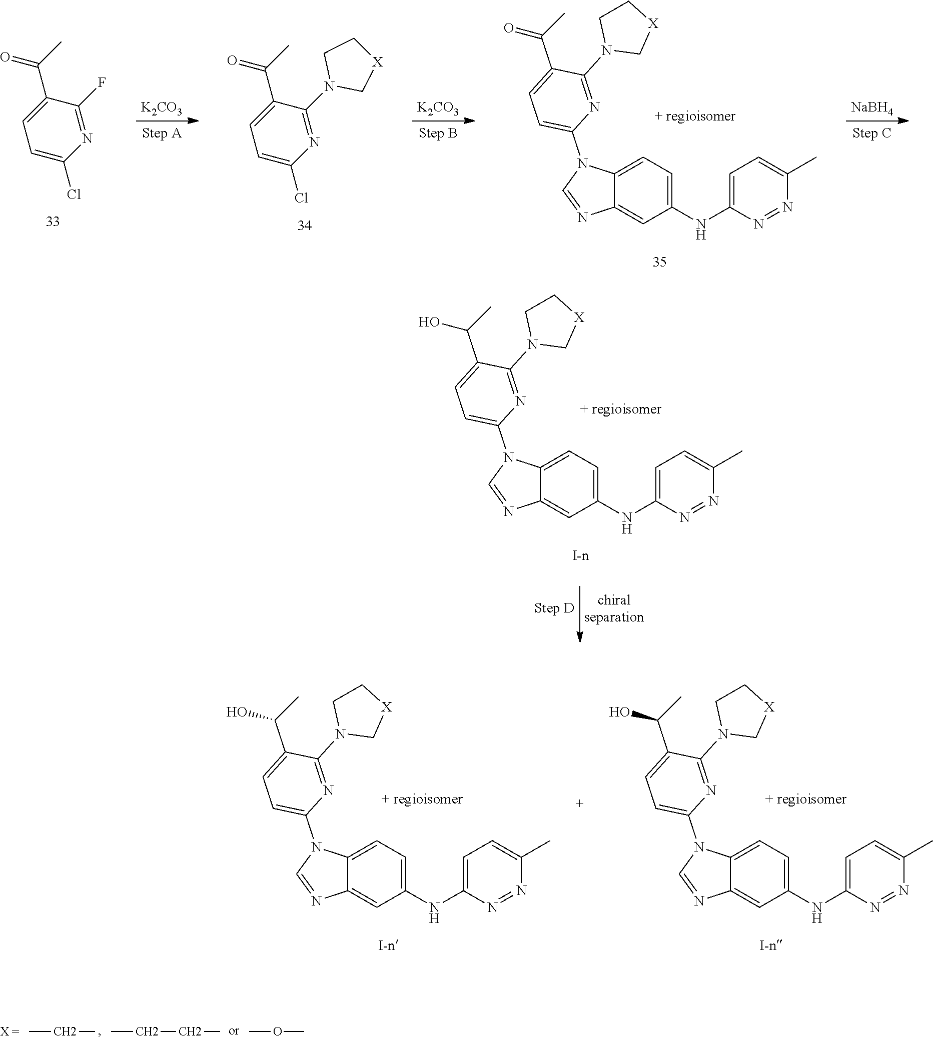

Step A: 1-(6-chloro-2-fluoro-3-pyridyl)ethanone 33 can be reacted with a cyclic amine (or sulfonamide or amide) in the presence of a suitable base such as for instance K₂CO₃ or Cs₂CO₃ in a suitable solvent such as e.g. DMSO to yield intermediate 34.

Step B: Intermediate 34 can be reacted in DMSO with N-(6-methylpyridazin-3-yl)-1H-benzimidazol-5-amine (intermediate 18 from Scheme 6) in the presence of K₂CO₃ or Cs₂CO₃ at 30° C.-50° C. to obtain intermediate 35 (and its regioisomer).

Step C: The secondary alcohols of compound of formula (I-n) can be obtained after reduction of the carbonyl of intermediate 35 with a suitable reducing agent such as for instance NaBH₄ (1-5 equiv.) in a suitable solvent such as for instance MeOH (and in some cases a second co-solvent, e.g. DCM, THF, DMF) at temperatures between around −40° C. and rt.

Step D: The enantiomers I-n′ and I-n″ of the secondary alcohol can be separated by chiral SFC.

### Scheme 11

In scheme 11, the synthesis of a compound of formula (I-o) is described. The compound of formula (I-o) is a compound of formula (I), wherein A1 is —NH—; A2 is a bond; A3 is —NH—: R¹ is hydrogen; R² is methyl; R^{2′} is hydrogen; R³ is alkyl; R⁴ is hydrogen; R⁵ is pyridazin-3-yl substituted with methyl; each R⁷ is independently selected from alkyl, cyano, halogen, haloalkyl, haloalkoxy, hydroxy, alkoxy, and cycloalkylamino.

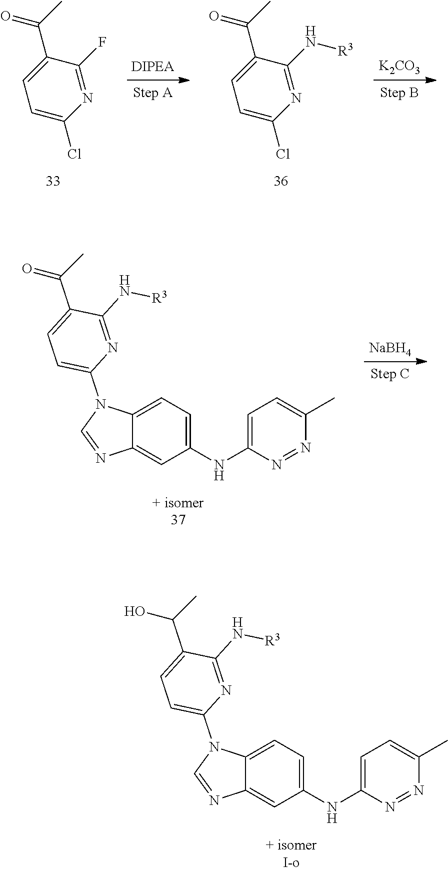

Step A: 1-(6-chloro-2-fluoro-3-pyridyl)ethanone 33 can be reacted with an aliphatic amine in the presence of DIPEA in DMSO or DMF or DME or NMP to yield intermediate 36.

Step B: Intermediate 36 can be reacted in DMSO with N-(6-methylpyridazin-3-yl)-1H-benzimidazol-5-amine (intermediate 18 from Scheme 6) in the presence of K₂CO₃ at 30° C. to obtain intermediate 37 (and its regioisomer).

Step C: The compound of formula (I-O) can be obtained after reduction of the carbonyl with a suitable reducing agent such as for instance NaBH₄ (1-5 equiv.) in a suitable solvent such as for instance MeOH (and in some cases a second co-solvent, e.g. DCM, THF, DMF) at temperatures between around −40° C. and rt.

The invention thus also relates to a process for the preparation of a compound according to the invention, comprising one of the following steps:
(a) the reaction of a compound of formula (B1)

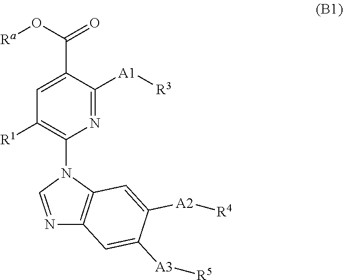

with a reducing agent;
(b) the reaction of a compound of formula (C1)

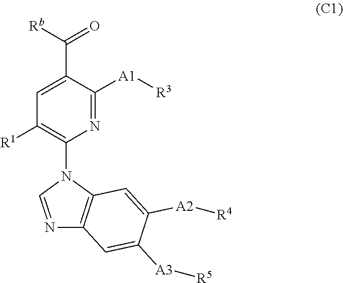

with a reducing agent; or
(c) the reaction of a compound (D1)

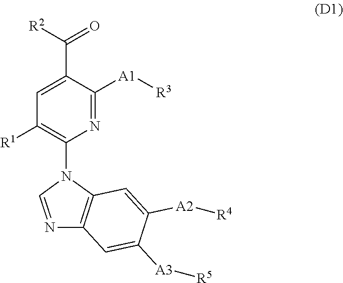

with a compound of formula R^{c}MgX,
wherein A1, A2, A3, R¹, R², R³, R⁴ and R⁵ are as defined above, R^{a} is alkyl or cycloalkyl, R^{b} is hydrogen or alkyl, R^{c} is alkyl or cycloalkyl, and X is halogen.

The reducing agent of step (a) can be LiAlH₄, DIBAL-H, The solvent of step (a) can advantageously be THF.

Convenient conditions for step (a) are between around −60° C. to around 40° C., in particular between around −50° C. to around 30° C., more particular between around −40° C. to around 20° C. during 1-24 hrs, advantageously during 1-12 hrs.

The reducing agent of step (b) can be for instance NaBH₄.

The solvent of step (b) can be for instance methanol or a mixture of methanol and THF.

Convenient conditions for step (b) are between around −60° C. to around 40° C., in particular between around −50° C. to around 30° C., more particular between around −40° C. to around 20° C. during 1-24 hrs, advantageously during 1-12 hrs.

In step (c) the solvent can be for instance THF, 2-MeTHF or a mixture thereof.

Convenient conditions for step (c) are between around −80° C. to around 30° C., in particular between around −78° C. to around 20° C., more particular between around −40° C. to around 0° C. during 1-24 hrs, advantageously during 1-12 hrs.

Conveniently, X is chloride, bromide or iodide. Advantageously, X is bromide.

The invention also relates to a compound according to the invention when manufactured according to a process of the invention.

### Pharmaceutical Compositions

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compound of formula (I) is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal, epidural and intranasal, and if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

### Example A

Film coated tablets containing the following ingredients can be manufactured in a conventional manner:

| Ingredients | Per tablet | |
|---|---|---|
| Kernel: | | |
| Compound of formula (I) or a | 10.0 mg | 200.0 mg |
| pharmaceutically acceptable salt thereof | | |
| Microcrystalline cellulose | 23.5 mg | 43.5 mg |
| Lactose hydrous | 60.0 mg | 70.0 mg |
| Povidone K30 | 12.5 mg | 15.0 mg |
| Sodium starch glycolate | 12.5 mg | 17.0 mg |
| Magnesium stearate | 1.5 mg | 4.5 mg |
| (Kernel Weight) | 120.0 mg | 350.0 mg |
| Film Coat: | | |
| Hydroxypropyl methyl cellulose | 3.5 mg | 7.0 mg |
| Polyethylene glycol 6000 | 0.8 mg | 1.6 mg |
| Talc | 1.3 mg | 2.6 mg |
| Iron oxide (yellow) | 0.8 mg | 1.6 mg |
| Titan dioxide | 0.8 mg | 1.6 mg |

The active ingredient is sieved and mixed with microcrystalline cellulose and the mixture is granulated with a solution of polyvinylpyrrolidone in water. The granulate is then mixed with sodium starch glycolate and magnesium stearate and compressed to yield kernels of 120 or 350 mg respectively. The kernels are lacquered with an aq. solution/suspension of the above mentioned film coat.

### Example B

Capsules containing the following ingredients can be manufactured in a conventional manner:

| Ingredients | Per capsule |
|---|---|
| Compound of formula (I) or a | 25.0 mg |
| pharmaceutically acceptable salt thereof | |
| Lactose | 150.0 mg |
| Maize starch | 20.0 mg |
| Talc | 5.0 mg |

The components are sieved and mixed and filled into capsules of size 2.

### Example C

Injection solutions can have the following composition:

| | |
|---|---|
| Compound of formula (I) or a | 3.0 mg |
| pharmaceutically acceptable salt thereof | |
| Polyethylene glycol 400 | 150.0 mg |
| Acetic acid | q.s. ad pH 5.0 |
| Water for injection solutions | ad 1.0 ml |

The active ingredient is dissolved in a mixture of Polyethylene glycol 400 and water for injection (part). The pH is adjusted to 5.0 by addition of acetic acid. The volume is adjusted to 1.0 ml by addition of the residual amount of water. The solution is filtered, filled into vials using an appropriate overage and sterilized.

### Experimental Procedures

### Abbreviations

[tBuBrettPhos Pd(allyl)]OTf allyl(2-di-tert-butylphosphino-3,6-dimethoxy-2′,4′,6′-triisopropyl-1,1′-biphenyl)palladium(II) triflate (CAS #1798782-15-6)
2-MeTHF 2-methyl tetrahydrofuran
ATP adenosine triphosphate
aq. aqueous
Boc tert-butyloxycarbonyl
CDI carbonyldiimidazole
DABCO 1,8-diazabicyclo[5.4.0]undec-7-ene
DAST diethylaminosulfur trifluoride
dba dibenzylideneacetone
DCM dichloromethane
DIAD diisopropyl azodicarboxylate
DIBAL-H diisobutylaluminium hydride
DIPEA N,N-diisopropylethylamine
DMF N,N-dimethylformamide
DMSO dimethyl sulfoxide
dppf 1,1′-ferrocenediyl-bis(diphenylphosphine)
dtbbpy 4,4′-Bis(1,1-dimethylethyl)-2,2′-bipyridine
dtbpy 4,4′-Di-tert-butyl-2,2′-dipyridyl
EDCI 1-ethyl-3-(3-dimethylaminopropyl)carbodiimide
equiv. equivalents
ESI electrospray ionization
Et ethyl
Et₂O diethyl ether
EtOAc ethyl acetate
EtOH ethanol
FA formic acid
HATU (1-[bis(dimethylamino)methylene]-1H-1,2,3-triazolo[4,5-b]pyridinium
3-oxide hexafluorophosphate
HMDS bis(trimethylsilyl)amine
HOBt hydoxybenzotriazole
HPLC high pressure liquid chromatography
ⁱPrOH isopropyl alcohol
Ir[dF(CF₃)ppy]2(dtbpy)(PF₆) [4,4′-bis(1,1-dimethylethyl)-2,2′-bipyridine-N1,N1′]bis[3,5-difluoro-2-[5-(trifluoromethyl)-2-pyridinyl-N]phenyl-C]iridium(III) hexafluorophosphate
LC-MS high-performance liquid chromatography
LDA lithium diisopropylamide
mCPBA meta-chloroperoxybenzoic acid
Me methyl
MeOH methanol
Ms methanesulfonyl
NMR nuclear magnetic resonance
NPLC normal phase liquid chromatography
PE petroleum ether
ppy 2-phenylpyridine
s psi pounds per square inch
PTSA para toluene sulfonic acid
Qphos 1,2,3,4,5-Pentaphenyl-1′-(di-tert-butylphosphino)ferrocene
RT room temperature
RuPhos-Pd-G3 (2-dicyclohexylphosphino-2′,6′-diisopropoxy-1,1′-biphenyl)[2-(2′-amino-1,1′-biphenyl)]palladium(II) methanesulfonate (CAS #1445085-77-7)
sat. saturated
SFC supercritical fluid chromatography
sol. solution
TBD triazabicyclodecene
TBDMS tert-butyldimethylsilyl
t-BuXphos-Pd-G3 [(2-Di-tert-butylphosphino-2′,4′,6′-triisopropyl-1,1′-biphenyl)-2-(2′-amino-1,1′-biphenyl)]palladium(II) methanesulfonate (CAS #1447963-75-8)
TEA triethylamine
Tf triflyl
TFA trifluoroacetic acid
TFAA trifluoroacetic acid anhydride
THF tetrahydrofuran
TLC thin layer chromatography

### Example 1

### (2-(3-chlorophenyl)-6-(5,6-dimethoxy-1H-benzo[d]imidazol-1-yl)pyridin-3-yl)methanol

Error! Objects cannot be created from editing field codes.

Step 1: methyl 2-chloro-6-(5,6-dimethoxy-1H-benzo[d]imidazol-1-yl)nicotinate
Error! Objects cannot be created from editing field codes.

In a 200 mL four-necked flask, NaH 60% dispersion in mineral oil (637 mg, 15.9 mmol, 2 equiv.) was combined with DMF (30 mL) to give a grey suspension. The mixture was cooled to 0° C. and 5,6-dimethoxy-1H-benzo[d]imidazole (1.42 g, 7.97 mmol, 1 equiv.) was added. The reaction mixture was stirred for 15 min. A solution of methyl 2,6-dichloronicotinate (1.64 g, 7.97 mmol, 1 equiv.) in DMF (10 mL) was added dropwise to the reaction mixture and stirring was continued for 15 min. The reaction mixture was quenched with 30 mL of 1 M HCl and a precipitate formed. The suspension was collected by filtration, washed with H₂O and dried under high vacuum. The title compound (1.414 g, 46.9% yield) was obtained as a light brown solid. LC-MS: m/z=348.1 [M+H]⁺, ESI pos.

### Step 2: methyl 2-(3-chlorophenyl)-6-(5,6-dimethoxy-1H-benzo[d]imidazol-1-yl)nicotinate

Error! Objects cannot be created from editing field codes.

In a microwave vial were added methyl 2-chloro-6-(5,6-dimethoxy-1H-benzo[d]imidazol-1-yl)nicotinate (1 g, 2.88 mmol, 1 equiv.), 2 M Na₂CO₃ (5 mL, 10.1 mmol, 3.5 equiv.), 1,2-dimethoxyethane (15 mL), (3-chlorophenyl)boronic acid (674 mg, 4.31 mmol, 1.5 equiv.) and Pd(PPh₃)₄(332 mg, 288 gmol, 0.1 equiv.). The vial was capped and heated in the microwave at 120° C. for 20 min. The reaction mixture was cooled to RT, diluted with 25 mL of H₂O and 25 mL of ethylacetate. The aqueous phase was back extracted with DCM. The combined organic layers were dried over Na₂SO₄, filtered and concentrated.

The residue was purified by flash chromatography (silica gel, 80 g, 0% to 5% MeOH in DCM). The chromatographied product was triturated in acetone, collected by filtration, washed with acetone and dried. The title compound (353 mg, 27.2% yield) was obtained as a light brown solid. LC-MS: m/z=424.2 [M+H]⁺, ESI pos.

### Step 3: (2-(3-chlorophenyl)-6-(5,6-dimethoxy-1H-benzo[d]imidazol-1-yl)pyridin-3-yl)methanol

Error! Objects cannot be created from editing field codes.

In a 100 mL round-bottomed flask, methyl 2-(3-chlorophenyl)-6-(5,6-dimethoxy-1H-benzo[d]imidazol-1-yl)nicotinate (317 mg, 748 gmol, 1 equiv.) was suspended in anhydrous THF (5 mL). The mixture was cooled to 0° C. and LiAlH₄ 1 M solution in THF (1.0 mL, 1 mmol, 1.34 equiv.) was added. Stirring at 0° C. was continued for 10 min. The reaction mixture was quenched with 1 M potassium sodium tartrate solution (25 mL) and diluted with DCM (50 mL). The layers were separated and the organic phase was concentrated to leave a yellow residue. This crude solid was purified by flash chromatography (silica gel, 25 g, 0-100% [DCM/MeOH/NH₄OH (95:5:1)] in DCM). The title compound (218 mg, 73.6% yield) was obtained as a white solid. LC-MS: m/z=396.2 [M+H]⁺, ESI pos.

### Example 15

### (2-(3-Chloro-2-fluorophenyl)-6-(5,6-dimethoxy-1H-benzo[d]imidazol-1-yl)pyridin-3-yl)methanol

Error! Objects cannot be created from editing field codes.

### Step 1: methyl 2-(3-chloro-2-fluorophenyl)-6-(5,6-dimethoxy-1H-benzo[d]imidazol-1-yl)nicotinate

Error! Objects cannot be created from editing field codes.

Starting from methyl 2-chloro-6-(5,6-dimethoxy-1H-benzo[d]imidazol-1-yl)nicotinate (obtained as in step 1 of example 1) (400 mg, 1.15 mmol, 1 equiv.) with (3-chloro-2-fluorophenyl)boronic acid (301 mg, 1.73 mmol, 1.5 equiv.), and following the procedure described in step 2 of example 2, the title compound (317 mg, 57.4% yield) was obtained as a light brown solid. LC-MS: m/z=442.2 [M+H]⁺, ESI pos.

### Step 2: (2-(3-chloro-2-fluorophenyl)-6-(5,6-dimethoxy-1H-benzo[d]imidazol-1-yl)pyridin-3-yl)methanol

Error! Objects cannot be created from editing field codes.

Following the procedure described in step 3 of example 1, the title compound (219 mg, 62.7% yield) was obtained as a light brown solid. LC-MS: m/z=414.2 [M+H]⁺, ESI pos.

### Example 16

(6-(5,6-Dimethoxy-1H-benzo[d]imidazol-1-yl)-2-(4-fluoro-2-methoxyphenyl)pyridin-3-yl)methanol
Error! Objects cannot be created from editing field codes.

### Step 1: methyl 6-(5,6-dimethoxy-1H-benzo[d]imidazol-1-yl)-2-(4-fluoro-2-methoxyphenyl)nicotinate

Error! Objects cannot be created from editing field codes.

Starting from methyl 2-chloro-6-(5,6-dimethoxy-1H-benzo[d]imidazol-1-yl)nicotinate (obtained as in step 1 of example 1) (500 mg, 1.15 mmol, 1 equiv.) with (4-fluoro-2-methoxyphenyl)boronic acid (367 mg, 2.16 mmol, 1.5 equiv.), and following the procedure described in step 2 of example 2, the title compound (498 mg, 64.1% yield) was obtained as an off-white solid. LC-MS: m/z=438.3 [M+H]⁺, ESI pos.

### Step 2: (6-(5,6-dimethoxy-1H-benzo[d]imidazol-1-yl)-2-(4-fluoro-2-methoxyphenyl)pyridin-3-yl)methanol

Error! Objects cannot be created from editing field codes.

Following the procedure described in step 3 of example 1, The title compound (398 mg, quantitative yield) was obtained as an off-white solid. LC-MS: m/z=410.2 [M+H]⁺, ESI pos.

### Example 17

### 1-(6-(5,6-Dimethoxy-1H-benzo[d]imidazol-1-yl)-2-(4-fluoro-2-methoxyphenyl)pyridin-3-yl)ethan-1-ol

Error! Objects cannot be created from editing field codes.

### Step 1: 6-(5,6-dimethoxy-1H-benzo[d]imidazol-1-yl)-2-(4-fluoro-2-methoxyphenyl)nicotinaldehyde

Error! Objects cannot be created from editing field codes.

In a 25 mL pear shape flask, (6-(5,6-dimethoxy-1H-benzo[d]imidazol-1-yl)-2-(4-fluoro-2-methoxyphenyl)pyridin-3-yl)methanol (398 mg, 972 gmol, 1 equiv.) (obtained as in step 2 of example 16) was suspended in DCM (5 mL). Dess-Martin periodinane (15% solution in DCM) (3.3 g, 2.4 mL, 1.17 mmol, 1.2 equiv.) was added. The reaction mixture soon turned to a light brown solution. A suspension formed a few moments later. After stirring for 10 min, the reaction mixture was quenched with H₂O, diluted with DCM and 2 M Na₂CO₃ was added. The layers were separated and the aqueous layer was back extracted with DCM. The combined organic layers were dried over Na₂SO₄, filtered and concentrated to leave a yellow residue. This crude material was purified by flash chromatography (silica gel, 40 g, 0% to 100% [DCM/MeOH/NH₄OH (95:5:1)] in DCM). The title compound (357 mg, 72.1% yield) was obtained as a yellow solid. LC-MS: m/z=408.2 [M+H]⁺, ESI pos.

### Step 2: 1-(6-(5,6-dimethoxy-1H-benzo[d]imidazol-1-yl)-2-(4-fluoro-2-methoxyphenyl)pyridin-3-yl)ethan-1-ol

Error! Objects cannot be created from editing field codes.

In a 25 mL pear shape flask, 6-(5,6-dimethoxy-1H-benzo[d]imidazol-1-yl)-2-(4-fluoro-2-methoxyphenyl)nicotinaldehyde (120 mg, 236 gmol, 1 equiv.) was suspended in anhydrous THF (2.5 mL). The mixture was cooled to 0° C. before MeMgBr (3.2 M solution in 2-MeTHF) (81 μL, 259 gmol, 1.1 equiv.) was added and the reaction mixture was stirred at 0° C. for 30 min. Additional MeMgBr (3.2 M solution in 2-MeTHF) (81 μL, 259 gmol, 1.1 equiv.) was added and stirring was continued for another 30 min. The reaction mixture was slowly quenched with H₂O (5 mL) at 0° C. Sat. aq. NH₄Cl solution (8 mL) was added and the mixture was diluted with DCM/MeOH 9:1. The layers were separated and the aqueous phase was back-extracted with DCM/MeOH 9:1. The combined organic layers were dried over Na₂SO₄, filtered and concentrated. The residue was purified by flash chromatography (silica gel, 25 g, 0% to 100% [DCM/MeOH/NH₄OH (95:5:1)] in DCM). The chromatographied product was dissolved in CH₃CN/H₂O 1:1 and freeze-dried. The title compound (90 mg, 88.4% yield) was obtained as a white lyophilized solid. LC-MS: m/z=424.3 [M+H]⁺, ESI pos.

### Example 18

### 1-(6-(5,6-Dimethoxy-1H-benzo[d]imidazol-1-yl)-2-(4-fluoro-2-methoxyphenyl)pyridin-3-yl)propan-1-ol

Error! Objects cannot be created from editing field codes.

Starting from 6-(5,6-dimethoxy-1H-benzo[d]imidazol-1-yl)-2-(4-fluoro-2-methoxyphenyl)nicotinaldehyde (obtained as in step 1 of example 17) (110 mg, 216 gmol, 1 equiv.) with EtMgBr (3.0 M solution in diethyl ether) (108 μL, 324 gmol, 1.5 equiv.) and following the procedure described in step 2 of example 17, the title compound (66 mg, 56.6% yield) was obtained as a white lyophilized solid. LC-MS: m/z=438.3 [M+H]⁺, ESI pos.

### Example 19

### 1-(2-(3-Chlorophenyl)-6-(5,6-dimethoxy-1H-benzo[d]imidazol-1-yl)pyridin-3-yl)ethan-1-ol

Error! Objects cannot be created from editing field codes.

### Step 1: 2-(3-chlorophenyl)-6-(5,6-dimethoxy-1H-benzo[d]imidazol-1-yl)nicotinaldehyde

Error! Objects cannot be created from editing field codes.

Starting from (2-(3-chlorophenyl)-6-(5,6-dimethoxy-1H-benzo[d]imidazol-1-yl)pyridin-3-yl)methanol (obtained as in step 3 of example 1) (218 mg, 551 gmol, 1 equiv.) and following the procedure described in step 1 of example 17, the title compound (250 mg, quantitative yield) was obtained as a yellow amorphous solid. LC-MS: m/z=394.1 [M+H]⁺, ESI pos.

### Step 2: 1-(2-(3-chlorophenyl)-6-(5,6-dimethoxy-1H-benzo[d]imidazol-1-yl)pyridin-3-yl)ethan-1-ol

Error! Objects cannot be created from editing field codes.

Following the procedure described in step 2 of example 17, the title compound (120 mg, 84.3% yield) was obtained as a white lyophilized solid. LC-MS: m/z=410.2 [M+H]⁺, ESI pos.

### Example 20

### 1-(2-(3-Chloro-2-fluorophenyl)-6-(5,6-dimethoxy-1H-benzo[d]imidazol-1-yl)pyridin-3-yl)ethan-1-ol

Error! Objects cannot be created from editing field codes.

### Step 1: methyl 2-(3-chloro-2-fluorophenyl)-6-(5,6-dimethoxy-1H-benzo[d]imidazol-1-yl)nicotinate

Error! Objects cannot be created from editing field codes.

Starting from methyl 2-chloro-6-(5,6-dimethoxy-1H-benzo[d]imidazol-1-yl)nicotinate (obtained as in step 1 of example 1) (400 mg, 1.15 mmol, 1 equiv.) with (3-chloro-2-fluorophenyl)boronic acid (301 mg, 1.73 mmol, 1.5 equiv.), and following the procedure described in step 2 of example 1, the title compound (317 mg, 57.4% yield) was obtained as a light brown solid. LC-MS: m/z=442.2 [M+H]⁺, ESI pos.

### Step 2: (2-(3-chloro-2-fluorophenyl)-6-(5,6-dimethoxy-1H-benzo[d]imidazol-1-yl)pyridin-3-yl)methanol

Error! Objects cannot be created from editing field codes.

Following the procedure described in step 3 of example 1, the title compound (219 mg, 62.7% yield) was obtained as a light brown solid. LC-MS: m/z=414.2 [M+H]⁺, ESI pos.

### Step 3: 2-(3-chloro-2-fluorophenyl)-6-(5,6-dimethoxy-1H-benzo[d]imidazol-1-yl)nicotinaldehyde

Error! Objects cannot be created from editing field codes.

Following the procedure described in step 1 of example 17, the title compound (137 mg, 49.1% yield) was obtained as a yellow solid. LC-MS: m/z=412.2 [M+H]⁺, ESI pos.

### Step 4: 1-(2-(3-chloro-2-fluorophenyl)-6-(5,6-dimethoxy-1H-benzo[d]imidazol-1-yl)pyridin-3-yl)ethan-1-ol

Error! Objects cannot be created from editing field codes.

Following the procedure described in step 2 of example 17, the title compound (104 mg, 66.5% yield) was obtained as a white lyophilized solid. LC-MS: m/z=428.2 [M+H]⁺, ESI pos.

### Example 21

(S)-1-(6-(5,6-Dimethoxy-1H-benzo[d]imidazol-1-yl)-2-(4-fluoro-2-methoxyphenyl)pyridin-3-yl)ethan-1-ol
Error! Objects cannot be created from editing field codes.

1-(6-(5,6-dimethoxy-1H-benzo[d]imidazol-1-yl)-2-(4-fluoro-2-methoxyphenyl)pyridin-3-yl)ethan-1-ol (60 mg, 142 gmol) (obtained as in step 2 of example 17) was purified by chiral SFC: column Chiral IA (250 mm×20 mm×5 μm). 30% (MeOH/EtOH/IprOH 1:1:1) in scCO₂. The title compound (22.5 mg, 36% yield) was obtained as a white lyophilized solid. LC-MS: m/z=424.2 [M+H]⁺, ESI pos.

### Example 22

### (R)-1-(6-(5,6-Dimethoxy-1H-benzo[d]imidazol-1-yl)-2-(4-fluoro-2-methoxyphenyl)pyridin-3-yl)ethan-1-ol

Error! Objects cannot be created from editing field codes.

1-(6-(5,6-dimethoxy-1H-benzo[d]imidazol-1-yl)-2-(4-fluoro-2-methoxyphenyl)pyridin-3-yl)ethan-1-ol (60 mg, 142 gmol) (obtained as in step 2 of example 17) was purified by chiral SFC: column Chiral IA (250 mm×20 mm×5 μm). 30% (MeOH/EtOH/IprOH 1:1:1) in scCO₂. The title compound (31.9 mg, 53.2% yield) was obtained as a white lyophilized solid. LC-MS: m/z=424.2 [M+H]⁺, ESI pos.

### Example 23

Cyclopropyl(6-(5,6-dimethoxy-1H-benzo[d]imidazol-1-yl)-2-(4-fluoro-2-methoxyphenyl)pyridin-3-yl)methanol
Error! Objects cannot be created from editing field codes.

Starting from 6-(5,6-dimethoxy-1H-benzo[d]imidazol-1-yl)-2-(4-fluoro-2-methoxyphenyl)nicotinaldehyde (obtained as in step 1 of example 17) (164 mg, 322 gmol, 1 equiv.) with cyclopropyl magnesium bromide (1 M solution in 2-MeTHF) (419 μL, 419 gmol, 1.3 equiv.) and following the procedure described in step 2 of example 17, the title compound (64.6 mg, 43.7% yield) was obtained as a white lyophilized solid. LC-MS: m/z=450.3 [M+H]⁺, ESI pos.

### Example 24

### 1-(2-(3-Chlorophenyl)-6-(5,6-dimethoxy-1H-benzo[d]imidazol-1-yl)pyridin-3-yl)propan-1-ol

Error! Objects cannot be created from editing field codes.

Starting from 2-(3-chlorophenyl)-6-(5,6-dimethoxy-1H-benzo[d]imidazol-1-yl)nicotinaldehyde (obtained as in step 1 of example 19) (125 mg, 286 gmol, 1 equiv.) and following the procedure described in example 18, the title compound (80 mg, 66.1% yield) was obtained as a white lyophilized solid. LC-MS: m/z=424.2 [M+H]⁺, ESI pos.

### Example 25

### (2-(3-Chlorophenyl)-6-(5,6-dimethoxy-1H-benzo[d]imidazol-1-yl)pyridin-3-yl)(cyclopropyl)methanol

Error! Objects cannot be created from editing field codes.

Starting from 2-(3-chlorophenyl)-6-(5,6-dimethoxy-1H-benzo[d]imidazol-1-yl)nicotinaldehyde (obtained as in step 1 of example 19) (89 mg, 226 gmol, 1 equiv.) and following the procedure described in example 23, the title compound (65 mg, 66% yield) was obtained as a white lyophilized solid. LC-MS: m/z=436.2 [M+H]⁺, ESI pos.

### Example 26

### (6-(5,6-Dimethoxy-1H-benzo[d]imidazol-1-yl)-2-morpholinopyridin-3-yl)methanol

Error! Objects cannot be created from editing field codes.

### Step 1: methyl 6-(5,6-dimethoxy-1H-benzo[d]imidazol-1-yl)-2-morpholinonicotinate

Error! Objects cannot be created from editing field codes.

A mixture of methyl 2-chloro-6-(5,6-dimethoxy-1H-benzo[d]imidazol-1-yl)nicotinate (174 mg, 0.5 mmol, 1 equiv.) (obtained as in step 1 of example 1) and morpholine (3.3 g, 3.3 mL, 37.9 mmol, 75.8 equiv.) in a sealed tube was heated to 100° C. and stirred for 1 hours. The reaction mixture was then cooled down to RT and purified by flash chromatography (silica gel, 20 g, 0-100% [DCM/MeOH (9:1)] in DCM). The title compound (128 mg, 61% yield) was obtained as a light brown solid. LC-MS: m/z=399.3 [M+H]⁺, ESI pos.

### Step 2: (6-(5,6-dimethoxy-1H-benzo[d]imidazol-1-yl)-2-morpholinopyridin-3-yl)methanol

Error! Objects cannot be created from editing field codes.

LiAlH₄ (11.4 mg, 300 gmol, 1 equiv.) was suspended in dry THF (0.3 mL). The suspension was cooled to 0° C. and a solution of methyl 6-(5,6-dimethoxy-1H-benzo[d]imidazol-1-yl)-2-morpholinonicotinate (120 mg, 0.3 mmol, 1 equiv.) in dry THF (1.6 mL) was added dropwise for 15 min while keeping the temperature at 0° C. Stirring at 0° C. was continued for 4 hours. The cooling bath was removed and the mixture was stirred at RT overnight. The mixture was cooled again to 0° C., carefully treated with H₂O and extracted with DCM. The organic layer was washed with brine, dried over MgSO₄, filtered and concentrated to dryness. The residue was purified by flash chromatography (silicagel, 10 g, 20-100% [DCM/MeOH (9:1)] in DCM). The chromatographied product was triturated in Et₂O (3 mL), collected by filtration, washed with Et₂O and dried. The tilte compound (51 mg, 45.9% yield) was obtained as a white solid. LC-MS: m/z=371.2 [M+H]⁺, ESI pos.

### Example 27

### (6-(5,6-Dimethoxy-1H-benzo[d]imidazol-1-yl)-2-(pyrrolidin-1-yl)pyridin-3-yl)methanol

Error! Objects cannot be created from editing field codes.

### Step 1: methyl 6-(5,6-dimethoxy-1H-benzo[d]imidazol-1-yl)-2-(pyrrolidin-1-yl)nicotinate

Error! Objects cannot be created from editing field codes.

A mixture of methyl 2-chloro-6-(5,6-dimethoxy-1H-benzo[d]imidazol-1-yl)nicotinate (174 mg, 0.5 mmol, 1 equiv.) (obtained as in step 1 of example 1), pyrrolidine (53 mg, 62 μL, 750 gmol, 1.5 equiv.) and K₂CO₃ (131 mg, 950 gmol, 1.9 equiv.) in DMSO (1.7 mL) was heated to 95° C. and stirred at that temperature for 3 hours. The mixture was cooled to RT and diluted with H₂O. A precipitate formed. The solid was collected by filtration and dissolved in DCM. The resulting solution was dried over MgSO₄, filtered and concentrated to leave the title compound (150 mg, 76.1% yield) as a white solid. LC-MS: m/z=383.2 [M+H]⁺, ESI pos.

### Step 2: (6-(5,6-dimethoxy-1H-benzo[d]imidazol-1-yl)-2-(pyrrolidin-1-yl)pyridin-3-yl)methanol

Error! Objects cannot be created from editing field codes.

Following the procedure described in step 2 of example 26, the title compound (86 mg, 56.7% yield) was obtained as an off-white solid. LC-MS: m/z=355.2 [M+H]⁺, ESI pos.

### Example 28

### (6-(5,6-Dimethoxy-1H-benzo[d]imidazol-1-yl)-2-(piperidin-1-yl)pyridin-3-yl)methanol

Error! Objects cannot be created from editing field codes.

### Step 1: methyl 6-(5,6-dimethoxy-1H-benzo[d]imidazol-1-yl)-2-(piperidin-1-yl)nicotinate

Error! Objects cannot be created from editing field codes.

Starting from 2-chloro-6-(5,6-dimethoxy-1H-benzo[d]imidazol-1-yl)nicotinate (obtained as in step 1 of example 1) (164 mg, 322 gmol, 1 equiv.) with piperidine (63.9 mg, 74.1 μL, 750 gmol, 1.5 equiv.) and following the procedure described in step 1 of example 27, the title compound (150 mg, 71.3% yield) was obtained as a light red solid. LC-MS: m/z=397.3 [M+H]⁺, ESI pos.

### Step 2: (6-(5,6-dimethoxy-1H-benzo[d]imidazol-1-yl)-2-(piperidin-1-yl)pyridin-3-yl)methanol

Error! Objects cannot be created from editing field codes.

Following the procedure described in step 2 of example 26, the title compound (93.9 mg, 61.7% yield) was obtained as an off-white solid. LC-MS: m/z=369.2 [M+H]⁺, ESI pos.

### Example 32

### 5-((6-(5,6-Dimethoxy-1H-benzo[d]imidazol-1-yl)-3-(hydroxymethyl)pyridin-2-yl)amino)pentan-1-ol

Error! Objects cannot be created from editing field codes.

### Step 1: methyl 6-chloro-2-(2-oxopiperidin-1-yl)nicotinate

Error! Objects cannot be created from editing field codes.

To a suspension of NaH 60% dispersion in mineral oil (208 mg, 5.2 mmol, 1 equiv.) in dry DMF (31 mL) was added piperidin-2-one (515 mg, 5.2 mmol, 1 equiv.) and the reaction mixture was stirred at 23° C. for 30 minutes. The reaction mixture was cooled to 0° C. and methyl 6-chloro-2-fluoronicotinate (986 mg, 5.2 mmol, 1 equiv.) was added. The cooling bath was removed and the solution was allowed to warm up to 23° C. Stirring was continued for 1 hours. The reaction mixture was added to ice-cold saturated aqueous NH₄Cl solution (100 mL). and extracted with EtOAc. The combined organics were washed with brine, dried over MgSO₄, filtered and concentrated to leave a light yellow liquid. This crude material was purified by flash chromatography (silicagel, 20 g, 0-80% heptane in EtOAc). The title compound (791 mg, 53.8% yield) was obtained as a light yellow solid. LC-MS: m/z=269.1 [M+H]⁺, ESI pos.

### Step 2: methyl 6-(5,6-dimethoxy-1H-benzo[d]imidazol-1-yl)-2-(2-oxopiperidin-1-yl)nicotinate

Error! Objects cannot be created from editing field codes.

A suspension of sodium hydride (60% dispersion in mineral oil) (20 mg, 0.5 mmol, 1.0 equiv.) in dry DMF (1.9 ml) was cooled to 0° C. and 5,6-dimethoxy-1H-benzo[d]imidazole (89.1 mg, 0.5 mmol, 1.0 equiv.) was added and the reaction mixture was stirred for 15 min. Methyl 6-chloro-2-(2-oxopiperidin-1-yl)nicotinate. (134 mg, 0.5 mmol, 1.0 equiv.) was dissolved in dry DMF (600 μl)) and the solution was added dropwise. Stirring was continued for 15 min at 0° C. then let to warm overnight to RT. The reaction mixture was cooled, and diluted with cold sat. aq. NH₄Cl sol. and extracted with EtOAc. The combined organic layers were washed with brine, dried over MgSO₄ and concentrated under reduced pressure. The crude product was purified by flash column chromatography (silica gel, 0-10% MeOH in DCM) to yield the title compound (62.5 mg, 28.9% yield) was obtained as an off-white solid. LC-MS: m/z=411.2 [M+H]⁺, ESI pos.

### Step 3: 5-((6-(5,6-Dimethoxy-1H-benzo[d]imidazol-1-yl)-3-(hydroxymethyl)pyridin-2-yl)amino)pentan-1-ol

Error! Objects cannot be created from editing field codes.

To a stirred solution of methyl 6-(5,6-dimethoxy-1H-benzo[d]imidazol-1-yl)-2-(2-oxopiperidin-1-yl)nicotinate (62 mg, 0.15 mmol, 1 equiv.) in a mixture of dry THF (560 μL) and ethanol (560 μL) was added calcium chloride (58 mg, 525 gmol, 3.5 equiv.). The reaction mixture was cooled to 0° C. NaBH₄ (25 mg, 675 gmol, 4.5 equiv.) was added in one portion and the mixture was stirred for 10 min. The cooling bath was removed and stirring at RT was continued for 2 hours. The mixture was poured into ice-cold sat. aq. NH₄Cl sol. (50 mL) and this was extracted with DCM. The organic layer was washed with brine, dried over MgSO₄, filtered and concentrated to dryness. The crude product was purified by prep. HPLC. The title compound (19.5 mg, 30.6% yield) was obtained as an off-white solid. LC-MS: m/z=387.2 [M+H]⁺, ESI pos. ¹H NMR (600 MHz, DMSO-d6): δ ppm 8.66 (s, 1H), 7.93 (s, 1H), 7.54 (d, J=7.8 Hz, 1H), 7.28 (s, 1H), 6.94 (d, J=7.7 Hz, 1H), 6.20 (t, J=5.3 Hz, 1H), 5.27 (s, 1H), 4.43 (d, J=5.3 Hz, 2H), 4.34 (s, 1H), 3.83 (d, J=6.4 Hz, 6H), 3.47-3.53 (m, 2H), 3.38 (d, J=5.1 Hz, 2H), 1.65 (br t, J=7.3 Hz, 2H), 1.42-1.48 (m, 2H), 1.35-1.41 (m, 2H).

### Example 33

### 1-(6-(5,6-Dimethoxy-1H-benzo[d]imidazol-1-yl)-3-(hydroxymethyl)pyridin-2-yl)pyrrolidin-2-one

Error! Objects cannot be created from editing field codes.

### Step 1: methyl 6-chloro-2-(2-oxopyrrolidin-1-yl)nicotinate

Error! Objects cannot be created from editing field codes.

Following the procedure described in step 1 of example 32 with pyrrolidin-2-one (443 mg, 5.2 mmol, 1 equiv.), the title compound (744 mg, 53.4% yield) was obtained as an orange liquid. LC-MS: m/z=255.1 [M+H]⁺, ESI pos.

### Step 2: methyl 6-(5,6-dimethoxy-1H-benzo[d]imidazol-1-yl)-2-(2-oxopyrrolidin-1-yl)nicotinate

Error! Objects cannot be created from editing field codes.

Following the procedure described in step 2 of example 32 (with reaction time of 21 hours at RT), the title compound (236 mg, 56.6% yield) was obtained as an off-white solid. LC-MS: m/z=397.2 [M+H]⁺, ESI pos.

### Step 3: 1-(6-(5,6-Dimethoxy-1H-benzo[d]imidazol-1-yl)-3-(hydroxymethyl)pyridin-2-yl)pyrrolidin-2-one

Error! Objects cannot be created from editing field codes.

Following the procedure described in step 2 of example 26 and starting from methyl 6-(5,6-dimethoxy-1H-benzo[d]imidazol-1-yl)-2-(2-oxopyrrolidin-1-yl)nicotinate (119 mg, 0.3 mmol), the title compound (93.9 mg, 61.7% yield) was obtained as an off-white solid. LC-MS: m/z=369.2 [M+H]⁺, ESI pos.

### Example 34

### 4-((6-(5,6-Dimethoxy-1H-benzo[d]imidazol-1-yl)-3-(hydroxymethyl)pyridin-2-yl)amino)butan-1-ol

Error! Objects cannot be created from editing field codes.

The title compound (19.1 mg, 16.2% yield) was isolated as a side-product during the preparation of example 33 and was obtained as an off-white solid. LC-MS: m/z=373.2 [M+H]⁺, ESI pos.

### Example 35

(S)-2-(6-(5,6-Dimethoxy-1H-benzo[d]imidazol-1-yl)-3-(l-hydroxyethyl)pyridin-2-yl)benzamide
Error! Objects cannot be created from editing field codes.

### Step 1: (2-chloro-6-(5,6-dimethoxy-1H-benzo[d]imidazol-1-yl)pyridin-3-yl)methanol

Error! Objects cannot be created from editing field codes.

Starting from methyl 2-chloro-6-(5,6-dimethoxy-1H-benzo[d]imidazol-1-yl)nicotinate (obtained as in step 1 of example 1) (1.18 g, 3.38 mmol) and following the procedure described in step 3 of example 1, the title compound (702 mg, 61% yield) was obtained as a brown solid. LC-MS: m/z=320.1 [M+H]⁺, ESI pos.

### Step 2: 2-chloro-6-(5,6-dimethoxy-1H-benzo[d]imidazol-1-yl)nicotinaldehyde

Error! Objects cannot be created from editing field codes.

Following the procedure described in step 1 of example 17, with a reaction time of 7 h, the title compound (752 mg, quantitative yield) was obtained as a light brown solid. LC-MS: m/z=318.1 [M+H]⁺, ESI pos.

### Step 3: 1-(2-chloro-6-(5,6-dimethoxy-1H-benzo[d]imidazol-1-yl)pyridin-3-yl)ethan-1-ol

Following the procedure described in step 2 of example 17, using methylmagnesium bromide solution (1.4M in THF/Toluene (1:3)), the title compound (702 mg, 87.1% yield) was obtained as a yellow amorphous solid. LC-MS: m/z=334.1 [M+H]⁺, ESI pos.

### Step 4: 1-(5-(1-((tert-butyldimethylsilyl)oxy)ethyl)-6-chloropyridin-2-yl)-5,6-dimethoxy-1H-benzo[d]imidazole

Error! Objects cannot be created from editing field codes.

In a 150 mL round-bottomed flask, 1-(2-chloro-6-(5,6-dimethoxy-1H-benzo[d]imidazol-1-yl)pyridin-3-yl)ethan-1-ol (702 mg, 2.1 mmol, 1 equiv.) was suspended in anhydrous DCM (15 mL). The mixture was sparged with argon before imidazole (286 mg, 4.21 mmol, 2 equiv.) and tert-butylchlorodimethylsilane (485 mg, 3.15 mmol, 1.5 equiv.) were added subsequently. The reaction mixture turned to a yellow suspension and it was stirred at 20° C. for 3 h, after which only a small conversion of the starting material to the desired product was observed. DMF (6 mL) was added to the mixture which turned to a solution.

Stirring was continued at 20° C. overnight. The reaction mixture was quenched with H₂O and extracted with DCM. The layers were separated and the aqueous phase was back-extracted using DCM. The combined organic layers were dried over Na₂SO₄, filtered and concentrated to dryness. The crude material was purified by flash chromatography (silica gel, 40 g, 0% to 100% EtOAc in heptane). The title compound (748 mg, 1.67 mmol, 79.4% yield) was obtained as a white solid. LC-MS: m/z=448.2 [M+H]⁺, ESI pos.

### Step 5: 2-(6-(5,6-dimethoxy-1H-benzo[d]imidazol-1-yl)-3-(1-hydroxyethyl)pyridin-2-yl)benzamide

Error! Objects cannot be created from editing field codes.

Following the procedure described in step 2 of example 1, using 1-(5-(1-((tert-butyldimethylsilyl)oxy)ethyl)-6-chloropyridin-2-yl)-5,6-dimethoxy-1H-benzo[d]imidazole (80 mg, 179 gmol, 1 equiv.) and (2-cyanophenyl)boronic acid (39.4 mg, 268 gmol, 1.5 equiv.) with a reaction time of 30 min at 130° C., the title compound (20 mg, 24.9% yield) was obtained as an off-white solid. LC-MS: m/z=419.2 [M+H]⁺, ESI pos.

### Step 6: (S)-2-(6-(5,6-dimethoxy-1H-benzo[d]imidazol-1-yl)-3-(-hydroxyethyl)pyridin-2-yl)benzamide

Error! Objects cannot be created from editing field codes.

2-(6-(5,6-dimethoxy-1H-benzo[d]imidazol-1-yl)-3-(1-hydroxyethyl)pyridin-2-yl)benzamide (20 mg, 48 gmol) was purified by chiral SFC: column Chiral OD-H (250 mm×20 mm×5 μm). Flow rate: 90 mL/min. 30% MeOH in scCO₂. The title compound (3.9 mg, 19.5% yield) was obtained as a white lyophilized solid. LC-MS: m/z=419.2 [M+H]⁺, ESI pos.

### Example 36

### (R)-2-(6-(5,6-Dimethoxy-1H-benzo[d]imidazol-1-yl)-3-(1-hydroxyethyl)pyridin-2-yl)benzamide

Error! Objects cannot be created from editing field codes.

2-(6-(5,6-dimethoxy-1H-benzo[d]imidazol-1-yl)-3-(1-hydroxyethyl)pyridin-2-yl)benzamide (obtained as in step 5 of example 35) (20 mg, 48 gmol) was purified by chiral SFC: column Chiral OD-H (250 mm×20 mm×5 μm). Flow rate: 90 mL/min. 30% MeOH in scCO₂. The title compound (6.5 mg, 32.5% yield) was obtained as a white lyophilized solid. LC-MS: m/z=419.2 [M+H]⁺, ESI pos.

### Example 37

(S)-3-(6-(5,6-Dimethoxy-1H-benzo[d]imidazol-1-yl)-3-(l-hydroxyethyl)pyridin-2-yl)benzonitrile
Error! Objects cannot be created from editing field codes.

### Step 1: 3-(6-(5,6-dimethoxy-1H-benzo[d]imidazol-1-yl)-3-(1-hydroxyethyl)pyridin-2-yl)benzonitrile

Error! Objects cannot be created from editing field codes.

Starting from 1-(5-(1-((tert-butyldimethylsilyl)oxy)ethyl)-6-chloropyridin-2-yl)-5,6-dimethoxy-1H-benzo[d]imidazole (obtained as in step 4 of example 35) (80 mg, 179 gmol, 1 equiv.) with (3-cyanophenyl)boronic acid (39 mg, 268 gmol, 1.5 equiv.) and following the procedure described in step 2 of example 1 with a reaction time of 30 min at 130° C., the title compound (60 mg, 83.9% yield) was obtained as a brown solid. LC-MS: m/z=401.1 [M+H]⁺, ESI pos.

### Step 2: (S)-3-(6-(5,6-dimethoxy-1H-benzo[d]imidazol-1-yl)-3-(1-hydroxyethyl)pyridin-2-yl)benzonitrile

Error! Objects cannot be created from editing field codes.

3-(6-(5,6-dimethoxy-1H-benzo[d]imidazol-1-yl)-3-(1-hydroxyethyl)pyridin-2-yl)benzonitrile (60 mg, 179 gmol) was purified by chiral SFC: column Chiral OD-H (250 mm×20 mm×5 μm). 30% MeOH in scCO₂. The title compound (5.3 mg, 8.8% yield) was obtained as a white lyophilized solid. LC-MS: m/z=401.2 [M+H]⁺, ESI pos.

### Example 38

### (R)-3-(6-(5,6-Dimethoxy-1H-benzo[d]imidazol-1-yl)-3-(l-hydroxyethyl)pyridin-2-yl)benzonitrile

Error! Objects cannot be created from editing field codes.

3-(6-(5,6-dimethoxy-1H-benzo[d]imidazol-1-yl)-3-(1-hydroxyethyl)pyridin-2-yl)benzonitrile (obtained as in step 1 of example 37) (60 mg, 179 gmol) was purified by chiral SFC: column Chiral OD-H (250 mm×20 mm×5 μm). 30% MeOH in scCO₂. The title compound (5.8 mg, 9.7% yield) was obtained as a white lyophilized solid. LC-MS: m/z=401.2 [M+H]⁺, ESI pos.

### Example 39

### (S)-4-(6-(5,6-Dimethoxy-1H-benzo[d]imidazol-1-yl)-3-(l-hydroxyethyl)pyridin-2-yl)benzonitrile

Error! Objects cannot be created from editing field codes.

### Step 1: 4-(6-(5,6-dimethoxy-1H-benzo[d]imidazol-1-yl)-3-(1-hydroxyethyl)pyridin-2-yl)benzonitrile

Error! Objects cannot be created from editing field codes.

Starting from 1-(5-(1-((tert-butyldimethylsilyl)oxy)ethyl)-6-chloropyridin-2-yl)-5,6-dimethoxy-1H-benzo[d]imidazole (obtained as in step 4 of example 35) (80 mg, 179 μmol, 1 equiv.) with (4-cyanophenyl)boronic acid (39 mg, 268 μmol, 1.5 equiv.) and following the procedure described in step 2 of example 1 with a reaction time of 30 min at 130° C., the title compound (26 mg, 36.4% yield) was obtained as an off-white solid. LC-MS: m/z=401.2 [M+H]⁺, ESI pos.

### Step 2: (S)-4-(6-(5,6-dimethoxy-1H-benzo[d]imidazol-1-yl)-3-(1-hydroxyethyl)pyridin-2-yl)benzonitrile

Error! Objects cannot be created from editing field codes.

4-(6-(5,6-dimethoxy-1H-benzo[d]imidazol-1-yl)-3-(1-hydroxyethyl)pyridin-2-yl)benzonitrile (26 mg, 65 μmol) was purified by chiral SFC: column Chiral OD-H (250 mm×20 mm×5 μm). 30% MeOH in scCO₂. The title compound (7.1 mg, 27.3% yield) was obtained as a white lyophilized solid. LC-MS: m/z=401.2 [M+H]⁺, ESI pos.

### Example 40

### (R)-4-(6-(5,6-Dimethoxy-1H-benzo[d]imidazol-1-yl)-3-(1-hydroxyethyl)pyridin-2-yl)benzonitrile

Error! Objects cannot be created from editing field codes.

4-(6-(5,6-dimethoxy-1H-benzo[d]imidazol-1-yl)-3-(1-hydroxyethyl)pyridin-2-yl)benzonitrile (obtained as in step 1 of example 39) (26 mg, 65 μmol) was purified by chiral SFC: column Chiral OD-H (250 mm×20 mm×5 μm). 30% MeOH in scCO₂. The title compound (8.5 mg, 32.7% yield) was obtained as a white lyophilized solid. LC-MS: m/z=401.2 [M+H]⁺, ESI pos.

### Example 41

### (S)-6-(5,6-Dimethoxy-1H-benzo[d]imidazol-1-yl)-3-(1-hydroxyethyl)-1′-methyl-[2,4′-bipyridin]-2′(1′H)-one

Error! Objects cannot be created from editing field codes.

### Step 1: 6-(5,6-dimethoxy-1H-benzo[d]imidazol-1-yl)-3-(1-hydroxyethyl)-1′-methyl-[2,4′-bipyridin]-2′(1′H)-one

Error! Objects cannot be created from editing field codes.

Starting from 1-(5-(1-((tert-butyldimethylsilyl)oxy)ethyl)-6-chloropyridin-2-yl)-5,6-dimethoxy-1H-benzo[d]imidazole (obtained as in step 4 of example 35) (80 mg, 179 μmol, 1 equiv.) with (1-methyl-2-oxo-1,2-dihydropyridin-4-yl)boronic acid (41 mg, 268 μmol, 1.5 equiv.) and following the procedure described in step 2 of example 1 with a reaction time of 30 min at 130° C., the title compound (35 mg, 45.8% yield) was obtained as a white lyophilized solid. LC-MS: m/z=407.2 [M+H]⁺, ESI pos.

### Step 2: (S)-6-(5,6-dimethoxy-1H-benzo[d]imidazol-1-yl)-3-(1-hydroxyethyl)-1′-methyl-[2,4′-bipyridin]-2′(1H)-one

Error! Objects cannot be created from editing field codes.

6-(5,6-dimethoxy-1H-benzo[d]imidazol-1-yl)-3-(1-hydroxyethyl)-1′-methyl-[2,4′-bipyridin]-2′(1′H)-one (35 mg, 86 μmol) was purified by chiral SFC: column Chiral OD-H (250 mm×20 mm×5 μm). 30% MeOH in scCO₂. The title compound (3.8 mg, 10.9% yield) was obtained as a white lyophilized solid. LC-MS: m/z=407.2 [M+H]⁺, ESI pos.

### Example 42

### (R)-6-(5,6-Dimethoxy-1H-benzo[d]imidazol-1-yl)-3-(1-hydroxyethyl)-1′-methyl-[2,4′-bipyridin]-2′(1′H)-one

Error! Objects cannot be created from editing field codes.

6-(5,6-dimethoxy-1H-benzo[d]imidazol-1-yl)-3-(1-hydroxyethyl)-1′-methyl-[2,4′-bipyridin]-2′(1′H)-one (obtained as in step 1 of example 41) (35 mg, 86 μmol) was purified by chiral SFC: column Chiral OD-H (250 mm×20 mm×5 μm). 30% MeOH in scCO₂. The title compound (11.3 mg, 32.3% yield) was obtained as a white lyophilized solid. LC-MS: m/z=407.2 [M+H]⁺, ESI pos.

### Example 53

### 1-[6-[5-(2-Morpholinoethoxy)benzimidazol-1-yl]-2-phenoxy-3-pyridyl]ethanol

Error! Objects cannot be created from editing field codes.

### Intermediate 1: 1-(6-chloro-2-phenoxy-3-pyridyl)ethanone

Error! Objects cannot be created from editing field codes.

To a solution of 1-(6-chloro-2-fluoro-3-pyridyl)ethanone (CAS #1260663-13-5, 500 mg, 2.88 mmol, 1 equiv.) in CH₃CN (8 mL) were added phenol (0.2 mL, 2.88 mmol, 1 equiv.) and K₂CO₃ (796 mg, 5.76 mmol, 2 equiv.). The reaction mixture was stirred at 30° C. for 12 hours. The reaction mixture was concentrated under reduced pressure. The residue was taken in EtOAc and H₂O. The aqueous phase was back-extracted with EtOAc. The combined organics were washed with brine, dried over anhydrous Na₂SO₄, filtered and concentrated. The residue was purified by flash chromatography (silicagel, 0% to 30% EtOAc in petroleum ether). The title compound (1.17 g, quantitative yield) was obtained as a colorless oil. LC-MS: m/z=248.0 [M+H]⁺, ESI pos.

### Intermediate 2, step 1: 4-[2-(4-nitrophenoxy)ethyl]morpholine

Error! Objects cannot be created from editing field codes.

To a stirred solution of 4-nitrophenol (5.0 g, 35.94 mmol, 1 equiv.), 2-morpholinoethanol (5.89 g, 35.94 mmol, 1 equiv.) and triphenylphosphine (10.37 g, 39.54 mmol, 1.1 equiv.) in THF (80 mL) was added dropwise a solution of DEAD (6.89 g, 39.54 mmol, 1.1 equiv.) in THF (20 mL) at 0° C. under a nitrogen atmosphere. The mixture was then stirred at 30° C. for 16 hours. The reaction mixture was poured into H₂O (200 mL) and extracted with ethyl acetate (3×200 mL). The combined organic layers were dried over anhydrous Na₂SO₄, filtered and concentrated in vacuo. The residue was purified by flash chromatography (silicagel, 10% MeOH in EtOAc). The title compound (3.6 g, 39.7% yield) was obtained as an off-white solid. LC-MS: m/z=253.0 [M+H]⁺, ESI pos. ¹H NMR (400 MHz, CD₃OD): δ=8.25-8.18 (m, 2H), 7.14-7.08 (m, 2H), 4.27 (t, J=5.4 Hz, 2H), 3.75-3.68 (m, 4H), 2.85 (t, J=5.5 Hz, 2H), 2.64-2.56 (m, 4H).

### Intermediate 2, step 2: 4-(2-morpholin-4-ylethoxy)aniline

Error! Objects cannot be created from editing field codes.

To a solution of 4-[2-(4-nitrophenoxy)ethyl]morpholine (3.5 g, 13.87 mmol, 1 equiv.) in MeOH (40 mL) was added 10% Pd/C (0.67 g, 0.630 mmol, 0.050 equiv.). The mixture was stirred at 25° C. for 16 h under H₂ (15 psi). The mixture was filtered and the filtrate was concentrated in vacuo to afford the crude title compound (2.7 g, 87.5% yield) as light yellow solid. LC-MS: m/z=223.1 [M+H]⁺, ESI pos.

### Intermediate 2, step 3: N-[4-(2-morpholinoethoxy)phenyl]acetamide

Error! Objects cannot be created from editing field codes.

To a stirred solution of 4-(2-morpholin-4-ylethoxy)aniline (2.7 g, 12.15 mmol, 1 equiv.) and triethylamine (5.1 mL, 36.44 mmol, 3 equiv.) in DCM (30 mL) was added acetic anhydride (1.49 g, 14.58 mmol, 1.2 equiv.) at 0° C. Then the mixture was stirred at 25° C. for 16 h under a nitrogen atmosphere. Then the mixture was poured into H₂O (100 mL) and extracted with EtOAc (3×100 mL). The combined organic layers were dried over anhydrous Na₂SO₄, filtered and concentrated in vacuo. The residue was purified by flash chromatography (silica gel, 50% to 100% EtOAc in petroleum ether). The title compound (2.2 g, 74% yield) was obtained as a white solid. LC-MS: m/z=265.1 [M+H]⁺, ESI pos. ¹H NMR (400 MHz, CDCl₃): δ=7.41-7.36 (m, 2H), 7.17 (br s, 1H), 6.90-6.83 (m, 2H), 4.10 (t, J=5.7 Hz, 2H), 3.77-3.73 (m, 4H), 2.81 (t, J=5.7 Hz, 2H), 2.62-2.57 (m, 4H), 2.16 (s, 3H).

### Intermediate 2, step 4: N-[4-(2-morpholinoethoxy)-2-nitro-phenyl]acetamide

Error! Objects cannot be created from editing field codes.

To a stirred solution of N-[4-(2-morpholinoethoxy)phenyl]acetamide (1.8 g, 6.81 mmol, 1 equiv.) in acetic anhydride (20 mL, 180.35 mmol, 26.48 equiv.) was added dropwise nitric acid (2.6 mL, 37.88 mmol, 5.56 equiv.) at 0° C. The cooling bath was removed and the mixture was stirred at 20° C. for another 1 hours. The mixture was quenched by the careful addition of ice-cold H₂O (150 mL). The mixture was then basified by the addition of 1 N NaOH until to pH 9 was reached. This was extracted with EtOAc (4×50 mL). The combined organic extracts were dried over anhydrous Na₂SO₄, filtered and concentrated in vacuo to afford the crude title compound (1.8 g, 85.4% yield) as a red oil. LC-MS: m/z=310.1 [M+H]⁺, ESI pos.

### Intermediate 2, step 5: 4-(2-morpholinoethoxy)-2-nitro-aniline

Error! Objects cannot be created from editing field codes.

To a stirred solution of of N-[4-(2-morpholinoethoxy)-2-nitro-phenyl]acetamide (1.8 g, 3.23 mmol, 1 equiv.) in a mixture of EtOH (15 mL) and H₂O (5 mL) was added potassium hydroxide (1.63 g, 29.1 mmol, 5 equiv.). The mixture was stirred at 80° C. for 4 hours. The reaction mixture was cold to RT and poured into H₂O (100 mL). This was extracted with EtOAc (3×50 mL). The combined organic layers were dried over anhydrous Na₂SO₄, filtered and concentrated in vacuo to afford the crude title compound (1.2 g, 77.1% yield) as red oil. LC-MS: m/z=268.2 [M+H]⁺, ESI pos. ¹H NMR (400 MHz, CDCl₃): δ=7.58 (d, J=2.9 Hz, 1H), 7.12-7.07 (m, 1H), 6.76 (d, J=9.0 Hz, 1H), 4.09 (t, J=5.6 Hz, 2H), 3.77-3.74 (m, 4H), 2.80 (t, J=5.6 Hz, 2H), 2.61-2.57 (m, 4H).

### Step 1: 1-[6-[4-(2-morpholinoethoxy)-2-nitro-anilino]-2-phenoxy-3-pyridyl]ethanone

Error! Objects cannot be created from editing field codes.

To a stirred solution of 4-(2-morpholinoethoxy)-2-nitro-aniline (intermediate 2 of example 53) (150 mg, 0.56 mmol, 1 equiv.) in 1,4-dioxane (3 mL) were added 1-(6-chloro-2-phenoxy-3-pyridyl)ethanone (intermediate 1 of example 53) (139 mg, 0.56 mmol, 1 equiv.), K₂CO₃ (233 mg, 1.68 mmol, 3 equiv.), Xantphos (130 mg, 0.22 mmol, 0.4 equiv.) and Pd2(dba)3 (103 mg, 0.110 mmol, 0.200 equiv.). The reaction mixture was stirred at 100° C. for 12 hours. The reaction mixture cooled to RT, poured into H₂O (30 mL) and extracted with EtOAc (3×30 mL). The combined organic layers were washed with brine (2×30 mL), dried over Na₂SO₄, filtered and concentrated under reduced pressure. The crude material was purified by flash chromatography (silica gel, 0% to 100% EtOAc in petroleum ether). The title compound (230 mg, 75.4% yield) was obtained as an orange solid. LC-MS: m/z=479.2 [M+H]⁺, ESI pos.

### Step 2: 1-[6-[2-amino-4-(2-morpholinoethoxy)anilino]-2-phenoxy-3-pyridyl]ethanone

Error! Objects cannot be created from editing field codes.

Following the procedure described in step 2, intermediate 2 of example 53, the title compound (160 mg, 94.8% yield) was obtained as light brown solid. LC-MS: m/z=449.2 [M+H]⁺, ESI pos.

### Step 3: 1-[6-[5-(2-morpholinoethoxy)benzimidazol-1-yl]-2-phenoxy-3-pyridyl]ethanone

Error! Objects cannot be created from editing field codes.

A solution of 1-[6-[2-amino-4-(2-morpholinoethoxy)anilino]-2-phenoxy-3-pyridyl]ethanone (140 mg, 0.31 mmol, 1 equiv.) in trimethyl orthoformate (10 mL, 6.24 mmol, 20 equiv.) was stirred at 125° C. for 24 hours. Then the reaction mixture was stirred at 130° C. for another 24 hours. The reaction mixture was cooled and concentrated to dryness. The residue was suspended in MeOH (10 mL). The solid was collected was collected by filtration, washed with MeOH and dried. The title compound (70 mg, 48.9% yield) was obtained as an off-white solid. LC-MS: m/z=479.2 [M+H]⁺, ESI pos.

### Step 4: 1-[6-[5-(2-morpholinoethoxy)benzimidazol-1-yl]-2-phenoxy-3-pyridyl]ethanol

Error! Objects cannot be created from editing field codes.

To a stirred solution of 1-[6-[5-(2-morpholinoethoxy)benzimidazol-1-yl]-2-phenoxy-3-pyridyl]ethanone (70 mg, 0.15 mmol, 1 equiv.) in MeOH (2 mL) was added NaBH₄ (17 mg, 0.46 mmol, 3 equiv.) at 0° C.

The cooling bath was removed and the mixture was stirred at 30° C. for 2 hours. The reaction mixture was quenched by the addition of saturated aq. NH₄Cl solution. This was extracted with EtOAc (3×10 mL). The combined organic layers were washed with brine, dried over anhydrous Na₂SO₄, filtered and concentrated. The crude material was purified by preparative HPLC. Column Phenomenex Gemini-NX C18 (75 mm×30 mm×3 μm). Flow rate 30 mL/min. Gradient: 24% to 54% CH₃CN in (10 mM NH₄HCO₃ in H₂O) (8 min) then 100% CH₃CN (2 min). The title compound (47.6 mg, 67% yield) was obtained as a white solid. LC-MS: m/z=461.2 [M+H]⁺, ESI pos. ¹H NMR (400 MHz, CD₃OD): δ=8.68 (s, 1H), 8.12 (d, J=8.1 Hz, 1H), 7.54-7.48 (m, 3H), 7.39-7.31 (m, 2H), 7.22 (br d, J=7.6 Hz, 2H), 7.14 (s, 1H), 6.64 (br d, J=9.4 Hz, 1H), 5.34-5.25 (m, 1H), 4.18-4.11 (m, 2H), 3.75-3.68 (m, 4H), 2.81 (br t, J=5.1 Hz, 2H), 2.60 (br s, 4H), 1.58 (d, J=6.4 Hz, 3H).

### Example 55

### 1-[2-Anilino-6-[5-(2-morpholinoethoxy)benzimidazol-1-yl]-3-pyridyl]ethanol

Error! Objects cannot be created from editing field codes.

### Step 1: 1-(2-anilino-6-chloro-3-pyridyl)ethanone

Error! Objects cannot be created from editing field codes.

To a stirred solution of 1-(6-chloro-2-fluoro-3-pyridyl)ethanone (CAS #1260663-13-5, 500 mg, 2.88 mmol, 1 equiv.) in CH₃CN (8 mL) was added aniline (0.26 mL, 2.88 mmol, 1 equiv.) and DIPEA (796 mg, 5.76 mmol, 2 equiv.). The reaction mixture was then stirred at 80° C. for 20 hours. The reaction mixture was cooled to RT, concentrated in vacuo and the residue was purified by flash chromatography (silica gel, 0% to 100% EtOAc in petroleum ether). The title compound (400 mg, 56.3% yield) was obtained as a yellow solid. LC-MS: m/z=247.0 [M+H]⁺, ESI pos.

### Step 2: 1-[2-anilino-6-[4-(2-morpholinoethoxy)-2-nitro-anilino]-3-pyridyl]ethanone

Error! Objects cannot be created from editing field codes.

Following the procedure described in step 2 of example 53, the title compound (200 mg, 51.7% yield) was obtained as a red solid. LC-MS: m/z=478.3 [M+H]⁺, ESI pos.

### Step 3: 1-[6-[2-amino-4-(2-morpholinoethoxy)anilino]-2-anilino-3-pyridyl]ethanone

Error! Objects cannot be created from editing field codes.

Following the procedure described in step 2, intermediate 2 of example 53, the title compound (160 mg, 85.3% yield) was obtained as a yellow oil. LC-MS: m/z=448.4 [M+H]⁺, ESI pos.

### Step 4: 1-[2-anilino-6-[5-(2-morpholinoethoxy)benzimidazol-1-yl]-3-pyridyl]ethanone

Error! Objects cannot be created from editing field codes.

Following the procedure described in step 3 of example 53, the title compound (90 mg, 80% yield) was obtained as a yellow oil. LC-MS: m/z=458.2 [M+H]⁺, ESI pos.

### Step 5: 1-[2-anilino-6-[5-(2-morpholinoethoxy)benzimidazol-1-yl]-3-pyridyl]ethanol

Error! Objects cannot be created from editing field codes.

Following the procedure described in step 4 of example 53, the title compound (34.1 mg, 42.4% yield) was obtained as a pink lyophilized solid after purification by preparative HPLC: Column Waters Xbridge (150 mm×25 mm×5 μm). Flow rate 25 mL/min. Gradient: 29% to 59% CH₃CN in (10 mM NH₄HCO₃ in H₂O) (9 min) then 100% CH₃CN (0.5 min). ¹H NMR (400 MHz, CD₃OD): δ=8.69 (s, 1H), 7.95 (d, 1H, J=9.0 Hz), 7.70 (d, 1H, J=7.8 Hz), 7.58 (d, 2H, J=7.7 Hz), 7.32 (t, 2H, J=7.9 Hz), 7.24 (d, 1H, J =2.1 Hz), 7.11 (d, 1H, J=7.8 Hz), 7.0-7.1 (m, 1H), 6.92 (dd, 1H, J=2.1, 9.1 Hz), 5.05 (q, 1H, J=6.4 Hz), 4.21 (t, 2H, J=5.4 Hz), 3.7-3.8 (m, 4H), 2.85 (t, 2H, J=5.4 Hz), 2.6-2.7 (m, 4H), 1.60 (d, 3H, J=6.5 Hz). LC-MS: m/z=460.4 [M+H]⁺, ESI pos.

### Example 62

### 1-[6-[5-(2-Morpholinoethoxy)benzimidazol-1-yl]-2-[3-(trifluoromethyl)pyrazol-1-yl]-3-pyridyl]ethanol

Error! Objects cannot be created from editing field codes.

### Step 1: 1-[6-chloro-2-[3-(trifluoromethyl)pyrazol-1-yl]-3-pyridyl]ethanone

Error! Objects cannot be created from editing field codes.

Following the procedure described in step 1 of example 64, with 3-(trifluoromethyl)pyrazole (400 mg, 2.94 mmol, 1.02 equiv.), the title compound (650 mg, 74% yield) was obtained as a light yellow solid. LC-MS: m/z=290.1 [M+H]⁺, ESI pos. ¹H NMR (400 MHz, CDCl₃): δ=8.55-8.49 (m, 1H), 7.80-7.73 (m, 1H), 7.42-7.36 (m, 1H), 6.79-6.73 (m, 1H), 2.41-2.35 (m, 3H).

### Step 2: 1-[6-[4-(2-morpholinoethoxy)-2-nitro-anilino]-2-[3-(trifluoromethyl)pyrazol-1-yl]-3-pyridyl]ethanone

Error! Objects cannot be created from editing field codes.

Following the procedure described in step 1 of example 53 (reaction time 16 h, temperature 90° C.), the title compound (210 mg, 31.3% yield) was obtained as a red gum. LC-MS: m/z=521.2 [M+H]⁺, ESI pos.

Purification by preparative HPLC: column Phenomenex Gemini-NX C18 (75 mm×30 mm×3 μm). Flow rate 25 mL/min. Gradient: 36% to 66% CH₃CN in (10 mM NH₄HCO₃ in H₂O) (10 min) then 100% CH₃CN (0.5 min).

### Step 3: 1-[6-[2-amino-4-(2-morpholinoethoxy)anilino]-2-[3-(trifluoromethyl)pyrazol-1-yl]-3-pyridyl]ethanone

Error! Objects cannot be created from editing field codes.

To a mixture of 1-[6-[4-(2-morpholinoethoxy)-2-nitro-anilino]-2-[3-(trifluoromethyl)pyrazol-1-yl]-3-pyridyl]ethanone (200 mg, 0.38 mmol, 1 equiv.) and NH₄Cl (205 mg, 3.83 mmol, 9.97 equiv.) in EtOH (10 mL) and H₂O (3 mL) was added Fe (108 mg, 1.93 mmol, 5.03 equiv.) in three portions at 20° C.

When the addition of Fe was complete, the resulting mixture was stirred at 50° C. for 16 hours. The black mixture was cooled to RT and filtered. The filtrate was concentrated and the residue was dissolved in 10 mL of CH₃CN. The resulting suspension was filtered. The filtrate was concentrated to give the title compound (180 mg, 95.5% yield) as a yellow solid. LC-MS: m/z=491.2 [M+H]⁺, ESI pos. ¹H NMR (400 MHz, CDCl₃): δ=8.31-8.26 (m, 1H), 7.76-7.70 (m, 1H), 7.10-7.04 (m, 1H), 6.78-6.72 (m, 1H), 6.43-6.41 (m, 1H), 6.36-6.29 (m, 3H), 4.59-4.45 (m, 2H), 4.22-4.02 (m, 4H), 3.49-2.92 (m, 6H), 2.16-2.10 (m, 3H).

### Step 4: 1-[6-[5-(2-morpholinoethoxy)benzimidazol-1-yl]-2-[3-(trifluoromethyl)pyrazol-1-yl]-3-pyridyl]ethanone

Error! Objects cannot be created from editing field codes

To a solution of 1-[6-[2-amino-4-(2-morpholinoethoxy)anilino]-2-[3-(trifluoromethyl)pyrazol-1-yl]-3-pyridyl]ethanone (180 mg, 0.37 mmol, 1 equiv.) in EtOH (5 mL) were added trimethoxymethane (389 mg, 3.67 mmol, 10 equiv.) and p-toluenesulfonic acid (6 mg, 0.040 mmol, 0.1 equiv.). The reaction mixture was stirred at 80° C. for 1 hour. The mixture was then cooled to RT and concentrated in vacuo. The residue was taken in DCM (20 mL) and washed with sat. aq. NaHCO₃. Sol. (20 mL). The organic layer was concentrated to dryness to afford the crude title compound (120 mg, 65.3% yield) as a light yellow solid. LC-MS: m/z=501.2 [M+H]⁺, ESI pos. ¹H NMR (400 MHz, CDCl₃): δ=8.53-8.50 (m, 1H), 8.50-8.47 (m, 1H), 8.01-7.98 (m, 1H), 7.95-7.91 (m, 1H), 7.56-7.50 (m, 1H), 7.32-7.28 (m, 1H), 7.05-7.01 (m, 1H), 6.79-6.76 (m, 1H), 4.70-4.30 (m, 2H), 4.04-3.73 (m, 4H), 3.37-2.77 (m, 6H), 2.36-2.31 (m, 3H).

### Step 5: 1-[6-[5-(2-morpholinoethoxy)benzimidazol-1-yl]-2-[3-(trifluoromethyl)pyrazol-1-yl]-3-pyridyl]ethanol

Error! Objects cannot be created from editing field codes.

1-[6-[5-(2-morpholinoethoxy)benzimidazol-1-yl]-2-[3-(trifluoromethyl)pyrazol-1-yl]-3-pyridyl]ethanone (50 mg, 0.1 mmol, 1 equiv.) was dissolved in DCM (1 mL) with stirring at 20° C. MeOH (0.5 mL) was added. The mixture was cooled to 0° C. NaBH₄ (15 mg, 0.4 mmol, 4 equiv.) was added portion-wise. The resulting mixture was stirred at 5° C. for 1 hour. The mixture was quenched by the addition of 5 drops of sat. aq. NH₄Cl sol., then formic acid was added until pH 7 was reached. The mixture was concentrated under reduced pressure. The residue was purified by preparative HPLC: column Phenomenex Gemini-NX C18 (75 mm×30 mm×3 μm). Flow rate 30 mL/min. Gradient: 28% to 58% CH₃CN in (10 mM NH₄HCO₃ in H₂O) (8 min) then 100% CH₃CN (2 min). The title compound (9.5 mg, 17.8% yield) was obtained as a colorless gum. LC-MS: m/z=503.2 [M+H]⁺, ESI pos. ¹H NMR (400 MHz, CD₃OD): δ=8.96-8.85 (m, 1H), 8.63-8.59 (m, 1H), 8.53-8.49 (m, 1H), 8.14-8.10 (m, 1H), 7.99-7.95 (m, 1H), 7.30-7.27 (m, 1H), 7.12-7.07 (m, 1H), 6.96-6.92 (m, 1H), 5.56-5.48 (m, 1H), 4.25-4.19 (m, 2H), 3.78-3.70 (m, 4H), 2.90-2.83 (m, 2H), 2.70-2.58 (m, 4H), 1.54-1.45 (m, 3H).

### Example 63

### 1-[6-(5,6-Dimethoxybenzimidazol-1-yl)-2-[3-(trifluoromethyl)pyrazol-1-yl]-3-pyridyl]ethanol

Error! Objects cannot be created from editing field codes.

### Step 1: 1-[6-(5,6-dimethoxybenzimidazol-1-yl)-2-[3-(trifluoromethyl)pyrazol-1-yl]-3-pyridyl]ethanone

Error! Objects cannot be created from editing field codes.

To a solution of 1-[6-chloro-2-[3-(trifluoromethyl)pyrazol-1-yl]-3-pyridyl]ethanone (obtained as in step 1 of example 62) (100 mg, 0.35 mmol, 1 equiv.) and 5,6-dimethoxy-1H-benzimidazole (62 mg, 0.35 mmol, 1 equiv.) in CH₃CN (5 mL) were added Cs₂CO₃ (225 mg, 0.69 mmol, 2 equiv.) and CsF (10 mg, 0.07 mmol, 0.19 equiv.). The mixture was degassed and purged with N₂ (three times). The mixture was then stirred at 70° C. for 16 h under N₂ atmosphere. The mixture then was cooled to 20° C. and filtered. The filtrate was purified directly purified by preparative HPLC: column Phenomenex Gemini-NX C18 (75 mm×30 mm×3 μm). Flow rate 30 mL/min. Gradient: 28% to 58% CH₃CN in (10 mM NH₄HCO₃ in H₂O) (8 min) then 100% CH₃CN (2 min). The title compound (25 mg, 12.6% yield) was obtained as light yellow solid. LC-MS: m/z=431.1 [M+H]⁺, ESI pos.

### Step 2: 1-[6-(5,6-dimethoxybenzimidazol-1-yl)-2-[3-(trifluoromethyl)pyrazol-1-yl]-3-pyridyl]ethanol

Error! Objects cannot be created from editing field codes.

Following the procedure described in step 5 of example 62, the title compound (8.5 mg, 39.7% yield) was obtained as a light yellow solid. Purification by preparative HPLC: column Phenomenex Gemini-NX C18 (75 mm×30 mm×3 μm). Flow rate 30 mL/min. Gradient: 28% to 58% CH₃CN in (10 mM NH₄HCO₃ in H₂O) (8 min) then 100% CH₃CN (2 min). LC-MS: m/z=434.1 [M+H]⁺, ESI pos. ¹H NMR (400 MHz, CD₃OD): δ=8.82-8.77 (m, 1H), 8.67-8.61 (m, 1H), 8.54-8.47 (m, 1H), 8.04-7.97 (m, 1H), 7.93-7.89 (m, 1H), 7.30-7.26 (m, 1H), 6.99-6.93 (m, 1H), 5.55-5.45 (m, 1H), 3.95-3.87 (m, 6H), 1.54-1.47 (m, 3H).

### Example 64

### 1-[3-(1-Hydroxyethyl)-6-[5-[(6-methylpyridazin-3-yl)amino]benzimidazol-1-yl]-2-pyridyl]-5-methyl-pyrazole-3-carbonitrile

Error! Objects cannot be created from editing field codes.

### Intermediate 1: N-(6-methylpyridazin-3-yl)-1H-benzimidazol-5-amine

Error! Objects cannot be created from editing field codes.

A mixture of 5-aminobenzimidazole (8.0 g, 60.1 mmol, 1.0 equiv.) and 3-chloro-6-methylpyridazine (7.34 g, 57.08 mmol, 0.950 equiv.) in ⁱPrOH (120 mL) was stirred at 120° C. for 72 hours. The dark brown suspension was concentrated in vacuo and the residue was triturated in MeOH (60 mL). The solid was collected by filtration and it was triturated in DCM (40 mL). The product was collected by filtration, washed with DCM and dried. The title compound (10 g, 71.3% yield) was obtained as brown solid. LC-MS: m/z=226.0 [M+H]⁺, ESI pos. ¹H NMR (400 MHz, DMSO-d6): δ=9.60 (br s, 1H), 8.72 (s, 1H), 8.52 (d, J=1.7 Hz, 1H), 7.63 (d, J=8.8 Hz, 1H), 7.44 (dd, J=2.0, 8.8 Hz, 1H), 7.39 (d, J=9.2 Hz, 1H), 7.21 (d, J=9.2 Hz, 1H), 5.04-4.15 (m, 1H), 2.49 (s, 3H).

### Step 1: 1-(3-acetyl-6-chloro-2-pyridyl)-5-methyl-pyrazole-3-carbonitrile

Error! Objects cannot be created from editing field codes.

A solution of 1-(6-chloro-2-fluoro-3-pyridyl)ethanone (CAS #1260663-13-5, 5 g, 28.81 mmol, 1 equiv.), 5-methyl-1H-pyrazole-3-carbonitrile (2.93 g, 27.37 mmol, 0.950 equiv.) and DIPEA (14.3 mL, 86.42 mmol, 3 equiv.) in DMSO (50 mL) was stirred at 80° C. for 4 hours. The reaction mixture was cooled to RT, poured into H₂O (250 mL) and extracted with EtOAc (3×150 mL). The combined organic layers were washed with brine (3×300 mL) and concentrated in vacuo. The residue was purified by flash chromatography (silica gel, 0% to 35% EtOAc in petroleum ether). The title compound (5.3 g, 70.6% yield) was obtained as yellow oil. LC-MS: m/z=261.1 [M+H]⁺, ESI pos.

### Step 2: 1-[3-acetyl-6-[5-[(6-methylpyridazin-3-yl)amino]benzimidazol-1-yl]-2-pyridyl]-5-methyl-pyrazole-3-carbonitrile and 1-[3-acetyl-6-[6-[(6-methylpyridazin-3-yl)amino]benzimidazol-1-yl]-2-pyridyl]-5-methyl-pyrazole-3-carbonitrile

Error! Objects cannot be created from editing field codes.

A mixture of 1-(3-acetyl-6-chloro-2-pyridyl)-5-methyl-pyrazole-3-carbonitrile (5.3 g, 20.33 mmol, 1 equiv.), N-(6-methylpyridazin-3-yl)-1H-benzimidazol-5-amine (intermediate 1) (4.58 g, 20.33 mmol, 1 equiv.) and K₂CO₃ (5.62 g, 40.66 mmol, 2 equiv.) in DMSO (50 mL) was stirred at 50° C. for 12 hours. The mixture was cooled to RT and poured into H₂O (500 mL). A solid precipitated out. This was extracted with EtOAc (3×400 mL). The combined organic layers were concentrated. The residue was purified by preparative HPLC: column Phenomenex Luna C18 (250 mm×70 mm×15 μm. Flow rate 140 mL/min. Gradient: 20% to 50% CH₃CN in (H₂O with 0.225% formic acid v/v) (35 min) then 100% CH₃CN (1 min). A mixture of the 2 title compounds was obtained. This mixture was purified by preparative NPLC: column Welch Ultimate XB-SiOH (250 mm×70 mm×10 μm). Flow rate 140 mL/min. Gradient: 20% to 60% EtOH in hexane (20 min) then 100% EtOH (3 min) to yield 1-[3-acetyl-6-[6-[(6-methylpyridazin-3-yl)amino]benzimidazol-1-yl]-2-pyridyl]-5-methyl-pyrazole-3-carbonitrile (1.1 g, 12% yield) as a light brown solid and 1-[3-acetyl-6-[5-[(6-methylpyridazin-3-yl)amino]benzimidazol-1-yl]-2-pyridyl]-5-methyl-pyrazole-3-carbonitrile (600 mg, 6.6% yield) as light brown solid. Regioisomer 1: LC-MS: m/z=450.1 [M+H]⁺, ESI pos. ¹H NMR (400 MHz, DMSO-d₆) δ=9.31 (s, 1H), 9.01 (s, 1H), 8.98 (d, J=2.0 Hz, 1H), 8.55 (d, J=8.4 Hz, 1H), 8.27 (d, J=8.4 Hz, 1H), 7.70 (d, J=8.7 Hz, 1H), 7.46 (dd, J=2.0, 8.7 Hz, 1H), 7.30 (d, J=9.0 Hz, 1H), 7.09-7.04 (m, 2H), 2.55 (s, 3H), 2.50 (br s, 3H), 2.19 (s, 3H). Regioisomer 2: LC-MS: m/z=450.1 [M+H]⁺, ESI pos. ¹H NMR (400 MHz, DMSO-d₆) δ=9.30 (s, 1H), 9.11 (s, 1H), 8.54 (d, J=8.4 Hz, 1H), 8.47 (d, J=1.7 Hz, 1H), 8.30 (d, J=8.6 Hz, 1H), 8.14 (d, J=8.9 Hz, 1H), 7.53 (dd, J=1.9, 8.9 Hz, 1H), 7.34 (d, J=9.0 Hz, 1H), 7.14 (s, 1H), 7.10 (d, J=9.0 Hz, 1H), 2.54 (s, 3H), 2.48 (br s, 3H), 2.20 (s, 3H).

### Step 3: 1-[3-(1-hydroxyethyl)-6-[5-[(6-methylpyridazin-3-yl)amino]benzimidazol-1-yl]-2-pyridyl]-5-methyl-pyrazole-3-carbonitrile

Error! Objects cannot be created from editing field codes.

To a solution of 1-[3-acetyl-6-[5-[(6-methylpyridazin-3-yl)amino]benzimidazol-1-yl]-2-pyridyl]-5-methyl-pyrazole-3-carbonitrile (600 mg, 1.33 mmol, 1 equiv.) in a mixture of MeOH (10 mL) and THF (10 mL) was added NaBH₄ (151 mg, 3.99 mmol, 2.99 equiv.) at 0° C. Stirring at 0° C. was continued for 1 hour. DMF (5 mL) was added to the mixture which was stirred at 20° C. for 2 hours. The mixture was cooled again to 0° C. and NaBH₄ (76 mg, 2 mmol, 1.5 equiv.) was added. The cooling bath was removed and stirring at RT was continued for 2 hours. The reaction mixture was quenched by the slow addition of H₂O (50 mL) and then extracted with DCM (2×50 mL). The combined organic extracts were concentrated under vacuum. The residue was purified by preparative HPLC: column Waters Xbridge (150 mm×25 mm×5 μm). Flow rate 60 mL/min. Gradient: 19% to 49% CH₃CN in (10 mM NH₄HCO₃ in H₂O) (11 min) then 100% CH₃CN (2 min). The title compound (400 mg, 65% yield) was obtained as a white liophilized solid. LC-MS: m/z=452.2 [M+H]⁺, ESI pos.

### Example 65

### 1-[3-(1-Hydroxyethyl)-6-[5-(4-methylpiperazin-1-yl)benzimidazol-1-yl]-2-pyridyl]-5-methyl-pyrazole-3-carbonitrile

Error! Objects cannot be created from editing field codes.

### Step 1: 1-[3-acetyl-6-(5-bromobenzimidazol-1-yl)-2-pyridyl]-5-methyl-pyrazole-3-carbonitrile and 1-[3-acetyl-6-(6-bromobenzimidazol-1-yl)-2-pyridyl]-5-methyl-pyrazole-3-carbonitrile

Error! Objects cannot be created from editing field codes.

To a solution of 5-bromo-1H-benzimidazole (1.51 g, 7.67 mmol, 1 equiv.) in DMSO (15 mL) were added 1-(3-acetyl-6-chloro-2-pyridyl)-5-methyl-pyrazole-3-carbonitrile (obtained as in step 1 of example 64) (2.0 g, 7.67 mmol, 1 equiv.) and K₂CO₃ (1.51 g, 15.34 mmol, 2 equiv.). The mixture was stirred at 60° C. for 2 hours. The reaction mixture was cooled to RT, poured into H₂O (100 mL) and extracted with EtOAc (3×100 mL). The combined organic layers were washed with brine (3×100 mL) and concentrated under vacuum. The residue was purified by flash chromatography (20% to 100% EtOAc in petroleum ether) to give a mixture of both title compounds. This mixture was purified by preparative HPLC: column Phenomenex Luna C18 (150 mm×40 mm×15 μm). Flow rate: 60 mL/min. Gradient: 53% to 63% CH₃CN in (H₂O with 0.225% formic acid v/v) (10 min) then 100% CH₃CN (2 min) to yield 1-[3-acetyl-6-(5-bromobenzimidazol-1-yl)-2-pyridyl]-5-methyl-pyrazole-3-carbonitrile (750 mg, 23.2% yield) as a light yellow liophilized solid and compound 1-[3-acetyl-6-(6-bromobenzimidazol-1-yl)-2-pyridyl]-5-methyl-pyrazole-3-carbonitrile (400 mg, 12.4% yield) as a light yellow lyophilized solid. Regioisomer 1: LC-MS: m/z=421.0 [M+H]⁺, ESI pos. ¹H NMR (400 MHz, CDCl₃) δ=8.60 (s, 1H), 8.32 (d, J=8.3 Hz, 1H), 8.25 (s, 1H), 7.81-7.73 (m, 2H), 7.56 (dd, J=1.6, 8.4 Hz, 1H), 6.74 (s, 1H), 2.67 (s, 3H), 2.25 (s, 3H). Regioisomer 2: LC-MS: m/z=421.0 [M+H]⁺, ESI pos. ¹H NMR (400 MHz, CDCl₃) δ=8.67-8.52 (m, 1H), 8.22 (d, J=8.3 Hz, 1H), 8.00 (br s, 1H), 7.90 (br d, J=5.0 Hz, 1H), 7.66 (d, J=8.3 Hz, 1H), 7.46 (d, J=8.8 Hz, 1H), 6.64 (s, 1H), 2.53 (s, 3H), 2.15 (s, 3H).

### Step 2: 1-[3-acetyl-6-[5-(4-methylpiperazin-1-yl)benzimidazol-1-yl]-2-pyridyl]-5-methyl-pyrazole-3-carbonitrile

Error! Objects cannot be created from editing field codes.

To a solution of 1-methylpiperazine (71 mg, 0.710 mmol, 2 equiv.) in 1,4-dioxane (5 mL) were added 1-[3-acetyl-6-(5-bromobenzimidazol-1-yl)-2-pyridyl]-5-methyl-pyrazole-3-carbonitrile (150 mg, 0.36 mmol, 1 equiv.), Cs₂CO₃ (348 mg, 1.07 mmol, 3 equiv.) and t-Buxphos-Pd-G3 (CAS #1447963-75-8) (28 mg, 0.04 mmol, 0.1 equiv.). The grey suspension was stirred at 90° C. for 4 h under N₂ atmosphere. The reaction mixture was cooled to RT, poured into H₂O (20 mL) and extracted with EtOAc (2×30 mL). The combined extracts were concentrated under vacuum. The residue was purified by preparative TLC (silica gel, 20% MeOH in DCM, UV detection). The title compound (30 mg, 19.1% yield) was obtained as a yellow solid. LC-MS: m/z=441.3 [M+H]⁺, ESI pos.

### Step 3: 1-[3-(1-hydroxyethyl)-6-[5-(4-methylpiperazin-1-yl)benzimidazol-1-yl]-2-pyridyl]-5-methyl-pyrazole-3-carbonitrile

Error! Objects cannot be created from editing field codes.

To a stirred solution of 1-[3-acetyl-6-[5-(4-methylpiperazin-1-yl)benzimidazol-1-yl]-2-pyridyl]-5-methyl-pyrazole-3-carbonitrile (30 mg, 0.07 mmol, 1 equiv.) in a mixture of MeOH (0.5 mL) and THF (0.5 mL)was added NaBH₄ (8 mg, 0.2 mmol, 3 equiv.) at 0° C. Stirring at 0° C. was continued for 1 hour. The reaction mixture was quenched by the addition of H₂O (10 mL). DCM (10 mL) was added to the reaction mixture. The aqueous phase was back-extracted with DCM (2×10 mL). The combined organic extracts were concentrated under vacuum. The residue was purified by prep-HPLC: column Waters Xbridge (150 mm×25 mm×5 μm). Flow rate: 25 mL/min. Gradient: 25% to 55% CH₃CN in (10 mM NH₄HCO₃ in H₂O) (10 min) then 100% CH₃CN (2 mmin). The title compound (4 mg, 13.2% yield) was obtained as a white lyophilized solid. LC-MS: m/z=443.2 [M+H]⁺, ESI pos. ¹H NMR (400 MHz, CD₃OD): δ=8.85 (s, 1H), 8.48 (d, J=8.4 Hz, 1H), 8.11 (d, J=8.6 Hz, 1H), 8.05 (d, J=9.2 Hz, 1H), 7.28 (d, J=2.1 Hz, 1H), 7.17 (dd, J=2.1, 9.1 Hz, 1H), 6.86 (s, 1H), 4.76 (q, J=6.6 Hz, 1H), 3.27-3.22 (m, 4H), 2.69-2.63 (m, 4H), 2.38 (d, J=10.0 Hz, 6H), 1.40 (d, J=6.4 Hz, 3H).

### Example 67

### 1-[3-(1-Hydroxyethyl)-6-[5-[4-(oxetan-3-yl)piperazin-1-yl]benzimidazol-1-yl]-2-pyridyl]-5-methyl-pyrazole-3-carbonitrile

Error! Objects cannot be created from editing field codes.

### Step 1: 1-[3-acetyl-6-[5-[4-(oxetan-3-yl)piperazin-1-yl]benzimidazol-1-yl]-2-pyridyl]-5-methyl-pyrazole-3-carbonitrile

Error! Objects cannot be created from editing field codes.

Following the procedure described in step 2 of example 65, starting with 1-(oxetan-3-yl)piperazine (101 mg, 0.71 mmol, 2 equiv.) and using H₂O (0.06 mL, 3.56 mmol, 10 equiv.) as additional reagent, the title compound (40 mg, 23.3% yield) was obtained as a yellow oil. LC-MS: m/z=473.2 [M+H]⁺, ESI pos.

### Step 2: 1-[3-(1-hydroxyethyl)-6-[5-[4-(oxetan-3-yl)piperazin-1-yl]benzimidazol-1-yl]-2-pyridyl]-5-methyl-pyrazole-3-carbonitrile

Error! Objects cannot be created from editing field codes.

Following the procedure described in step 3 of example 65, at a temperature of −40° C. and a reaction time of 30 min, the title compound (15.6 mg, 38.1% yield) was obtained as a white lyophilized solid. LC-MS: m/z=485.2 [M+H]⁺, ESI pos.

Purification by preparative HPLC: column Pheomenex Gemini-NX C18 (75 mm×30 mm×3 μm). Flow rate: 30 mL/min. Gradient: 15% to 45% CH₃CN in (10 mM NH₄HCO₃ in H₂O) (8 min) then 100% CH₃CN (2 min). ¹H NMR (400 MHz, CD₃OD): δ=8.83 (s, 1H), 8.47 (d, J=8.6 Hz, 1H), 8.11-8.00 (m, 2H), 7.26 (d, J=2.1 Hz, 1H), 7.14 (dd, J=2.1, 9.1 Hz, 1H), 6.85 (s, 1H), 4.79-4.70 (m, 3H), 4.67-4.62 (m, 2H), 3.57 (quin, J=6.4 Hz, 1H), 3.24 (br s, 4H), 2.55 (br s, 4H), 2.38 (s, 3H), 1.40 (d, J=6.5 Hz, 3H).

### Example 68

### 1-[3-(1-Hydroxyethyl)-6-[5-(2-morpholinoethoxy)benzimidazol-1-yl]-2-pyridyl]-5-methyl-pyrazole-3-carbonitrile

Error! Objects cannot be created from editing field codes.

### Step 1:1-[3-acetyl-6-[5-(2-morpholinoethoxy)benzimidazol-1-yl]-2-pyridyl]-5-methyl-pyrazole-3-carbonitrile

Error! Objects cannot be created from editing field codes.

Starting with of 2-morpholinoethanol (124 mg, 0.95 mmol, 2 equiv.) and following the procedure described in step 2 of example 65, the title compound (30 mg, 13.4% yield) was obtained as yellow oil. LC-MS: m/z=472.3 [M+H]⁺, ESI pos.

### Step 2: 1-[3-(1-hydroxyethyl)-6-[5-(2-morpholinoethoxy)benzimidazol-1-yl]-2-pyridyl]-5-methyl-pyrazole-3-carbonitrile

Error! Objects cannot be created from editing field codes.

Following the procedure described in step 3 of example 65, at −40° C. for 2 h, then at −10° C. for 10 min, the title compound (6.8 mg, 22.6% yield) was obtained as a white lyophilized solid. LC-MS: m/z=474.3 [M+H]⁺, ESI pos.

Purification by preparative HPLC: column Waters Xbridge (150 mm×25 mm×5 μm). Flow rate: 25 mL/min. Gradient: 28% to 58% CH₃CN in (10 mM NH₄HCO₃ in H₂O) (10 min) then 100% CH₃CN (2 min). ¹H NMR (400 MHz, CD₃OD): δ=8.88 (s, 1H), 8.49 (d, J=8.5 Hz, 1H), 8.12 (d, J=8.5 Hz, 1H), 8.07 (d, J=9.0 Hz, 1H), 7.29 (d, J=2.1 Hz, 1H), 7.07 (dd, J=1.8, 8.9 Hz, 1H), 6.86 (s, 1H), 4.76 (q, J=6.4 Hz, 1H), 4.22 (t, J=5.4 Hz, 2H), 3.75-3.70 (m, 4H), 2.85 (t, J=5.4 Hz, 2H), 2.66-2.59 (m, 4H), 2.39 (s, 3H), 1.40 (d, J=6.4 Hz, 3H).

### Example 71

### 1-[3-(1-Hydroxyethyl)-6-[5-[[1-(oxetan-3-yl)pyrazol-4-yl]amino]benzimidazol-1-yl]-2-pyridyl]-5-methyl-pyrazole-3-carbonitrile

Error! Objects cannot be created from editing field codes.

### Step 1: 1-[6-(5-bromobenzimidazol-1-yl)-3-(-hydroxyethyl)-2-pyridyl]-5-methyl-pyrazole-3-carbonitrile

Error! Objects cannot be created from editing field codes.

Starting from 1-[3-acetyl-6-(5-bromobenzimidazol-1-yl)-2-pyridyl]-5-methyl-pyrazole-3-carbonitrile (obtained as in step 1 of example 65) (750 mg, 1.78 mmol, 1 equiv.) and following the procedure described in step 3 of example 65, the crude (after extraction work-up) title compound (750 mg, 99.5% yield) was obtained as an off-white solid. LC-MS: m/z=425.0 [M+H]⁺, ESI pos. ¹H NMR (400 MHz, CDCl₃): δ=8.50 (s, 1H), 8.32 (d, J=8.4 Hz, 1H), 7.96 (d, J=1.8 Hz, 1H), 7.82 (d, J=8.8 Hz, 1H), 7.69 (d, J=8.4 Hz, 1H), 7.44 (dd, J=1.8, 8.8 Hz, 1H), 6.64 (s, 1H), 4.81 (q, J=6.5 Hz, 1H), 2.42-2.39 (m, 3H), 1.45 (d, J=6.5 Hz, 3H).

### Step 2: 1-[3-(-hydroxyethyl)-6-[5-[[1-(oxetan-3-yl)pyrazol-4-yl]amino]benzimidazol-1-yl]-2-pyridyl]-5-methyl-pyrazole-3-carbonitrile

Error! Objects cannot be created from editing field codes.

To a solution of 1-[6-(5-bromobenzimidazol-1-yl)-3-(1-hydroxyethyl)-2-pyridyl]-5-methyl-pyrazole-3-carbonitrile (100 mg, 0.24 mmol, 1 equiv.) in 1,4-dioxane (5 mL) were added 1-(3-oxetanyl)-1H-pyrazol-4-amine (39 mg, 0.280 mmol, 1.2 equiv.), Cs₂CO₃ (154 mg, 0.47 mmol, 2 equiv.) and RuPhos-Pd-G3 (20 mg, 0.02 mmol, 0.1 equiv.). The reaction mixture was stirred for 4 hours at 100° C. under N₂ atmosphere.

The mixture was cooled to RT, diluted with MeOH (20 mL), treated with thiourea resin and stirred at RT for 16 hours. The mixture was filtered and the filtrate was concentrated to dryness. The residue was purified by preparative HPLC: column Phenomenex Luna C18 (150 mm×25 mm×10 μm). Flow rate: 25 mL/min. Gradient: 16% to 46% CH₃CN in (0.225% formic acid in H₂O v/v) (10 min) then 100% CH₃CN (2 min). The title compound (57.1 mg, 45.2% yield) was obtained as an off-white lyophilized solid. LC-MS: m/z=482.2 [M+H]⁺, ESI pos. ¹H NMR (400 MHz, CD₃OD): δ=8.79 (s, 1H), 8.46 (d, J=8.5 Hz, 1H), 8.09 (d, J=8.5 Hz, 1H), 7.96 (d, J=8.9 Hz, 1H), 7.78 (s, 1H), 7.54 (s, 1H), 7.14 (d, J=2.1 Hz, 1H), 6.96 (dd, J=2.3, 8.9 Hz, 1H), 6.85 (d, J=0.6 Hz, 1H), 5.52 (quin, J=6.9 Hz, 1H), 5.05 (d, J=6.9 Hz, 4H), 4.75 (q, J=6.4 Hz, 1H), 2.39 (s, 3H), 1.40 (d, J=6.5 Hz, 3H).

### Example 72

### 1-[6-[5-[[1-(Azetidin-3-yl)pyrazol-4-yl]amino]benzimidazol-1-yl]-3-(1-hydroxyethyl)-2-pyridyl]-5-methyl-pyrazole-3-carbonitrile

Error! Objects cannot be created from editing field codes.

### Step 1: tert-butyl 3-[4-[[1-[6-(3-cyano-5-methyl-pyrazol-1-yl)-5-(-hydroxyethyl)-2-pyridyl]benzimidazol-5-yl]amino]pyrazol-1-yl]azetidine-1-carboxylate

Error! Objects cannot be created from editing field codes.

To a solution of 1-[6-(5-bromobenzimidazol-1-yl)-3-(1-hydroxyethyl)-2-pyridyl]-5-methyl-pyrazole-3-carbonitrile (140 mg, 0.33 mmol, 1 equiv.) in 1,4-dioxane (5 mL) were added tert-butyl 3-(4-aminopyrazol-1-yl)azetidine-1-carboxylate (95 mg, 0.4 mmol, 1.2 equiv.), Cs₂CO₃ (216 mg, 0.66 mmol, 2 equiv.) and RuPhos-Pd-G3 (28 mg, 0.03 mmol, 0.1 equiv.). The reaction mixture was heated to 100° C. and stirred for 4 hours under N₂ atmosphere. The reaction mixture was poured into H₂O (50 mL) and extracted with EtOAc (3×50 mL). The combined organics were washed with brine (2×50 mL) and concentrated in vacuo. The residue was purified by preparative TLC (silica gel, 10% MeOH in DCM, UV detection). The title compound (150 mg, 78.1% yield) was obtained as a brown oil. LC-MS: m/z=581.4 [M+H]⁺, ESI pos.

### Step 2: 1-[6-[5-[[1-(azetidin-3-yl)pyrazol-4-yl]amino]benzimidazol-1-yl]-3-(1-hydroxyethyl)-2-pyridyl]-5-methyl-pyrazole-3-carbonitrile

Error! Objects cannot be created from editing field codes.

To a solution of tert-butyl 3-[4-[[1-[6-(3-cyano-5-methyl-pyrazol-1-yl)-5-(1-hydroxyethyl)-2-pyridyl]benzimidazol-5-yl]amino]pyrazol-1-yl]azetidine-1-carboxylate (140 mg, 0.24 mmol, 1 equiv.) in DCM (2 mL) was added HCl in 1,4-dioxane (2.0 mL, 8 mmol, 33.18 equiv.). The reaction mixture was stirred at 30° C. for 2 hours. The reaction mixture was concentrated under reduced pressure and the residue was purified by preparative HPLC: column Phenomenex Luna C18 (150 mm×25 mm×10 μm).

Flow rate: 25 mL/min. Gradient: 6% to 36% CH₃CN in (0.225% formic acid in H₂O v/v) (10 min) then 100% CH₃CN (2 min). The title compound (27.9 mg, 22.9% yield) was obtained as a white lyophilized solid. LC-MS: m/z=481.2 [M+H]⁺, ESI pos. ¹H NMR (400 MHz, DMSO-d₆): δ=8.93 (s, 1H), 8.42 (d, J=8.6 Hz, 1H), 8.32 (s, 1H), 8.23 (d, J=8.4 Hz, 1H), 7.94-7.88 (m, 2H), 7.68 (s, 1H), 7.46 (s, 1H), 7.10 (s, 2H), 6.91 (dd, J=1.9, 8.8 Hz, 1H), 5.26-5.16 (m, 1H), 4.55 (q, J=6.4 Hz, 1H), 4.10-4.04 (m, 2H), 3.92 (br t, J=8.3 Hz, 2H), 2.34 (s, 3H), 1.26 (br d, J=6.4 Hz, 3H).

### Example 74

### 1-[3-(1-Hydroxyethyl)-6-[6-(6-methylpyridazin-3-yl)oxybenzimidazol-1-yl]-2-pyridyl]-5-methyl-pyrazole-3-carbonitrile

Error! Objects cannot be created from editing field codes.

### Step 1: 1-(2-trimethylsilylethoxymethyl)benzimidazol-5-ol

Error! Objects cannot be created from editing field codes.

A mixture of 1H-benzimidazol-5-ol (1.0 g, 7.45 mmol, 1 equiv.), 2-(trimethylsilyl)ethoxymethyl chloride (1.98 mL, 11.18 mmol, 1.5 equiv.) and DIPEA (1.93 g, 14.91 mmol, 2 equiv.) in DCM (30 mL) was stirred at 0° C. for 10 minutes, and then at 40° C. for 16 hours. More 2-(trimethylsilyl)ethoxymethyl chloride (0.66 mL, 3.73 mmol, 0.5 equiv.) was added and stirring at 40° C. was continued for 12 hours. The reaction mixture was cooled to RT, quenched with H₂O (150 mL) and extracted with DCM (3×50 mL). The combined organic layers were dried over Na₂SO₄, filtered and concentrated under reduced pressure. The residue was purified by flash chromatography (silica gel, 10% MeOH in DCM). The title compound (600 mg, 30.4% yield) was obtained as a yellow oil. LC-MS: m/z=265.1 [M+H]⁺, ESI pos.

### Step 2: trimethyl-[2-[[5-(6-methylpyridazin-3-yl)oxybenzimidazol-1-yl]methoxy]ethyl]silane

Error! Objects cannot be created from editing field codes.

A mixture of 1-(2-trimethylsilylethoxymethyl)benzimidazol-5-ol (0.6 g, 2.27 mmol, 1 equiv.), 3-chloro-6-methylpyridazine (292 mg, 2.27 mmol, 1 equiv.) and K₂CO₃ (0.63 g, 4.54 mmol, 2 equiv.) in DMF (10 mL) was stirred at 140° C. for 12 hours. The mixture was cooled to RT, quenched with H₂O (100 mL) and extracted with EtOAc (3×50 mL). The combined organic layers were washed with brine (3×30 mL), dried over Na₂SO₄, filtered and concentrated under reduced pressure. The residue which was purified by flash chromatography(silica gel, 10% MeOH in DCM. The title compound (800 mg, 79.1% yield) was obtained as a brown oil. LC-MS: m/z=357.1 [M+H]⁺, ESI pos.

### Step 3: 5-(6-methylpyridazin-3-yl)oxy-1H-benzimidazole

Error! Objects cannot be created from editing field codes.

A solution of trimethyl-[2-[[5-(6-methylpyridazin-3-yl)oxybenzimidazol-1-yl]methoxy]ethyl]silane (1.1 g, 3.09 mmol, 1 equiv.) in TFA (5 mL, 61.62 mmol, 19.97 equiv.) was stirred at 20° C. for 2 hours. The reaction mixture was quenched with H₂O (10 mL) and extracted with EtOAc (3×10 mL). The aqueous layer was freeze-dried to give the title compound (900 mg, TFA salt) as a yellow gum. This product was taken in DCM (100 mL) and treated with K₂CO₃. Stirring at at 30° C. was continued for 2 hours. The mixture was filtered and the filtrate was concentrated. The residue was purified by preparative HPLC: column Waters Xbridge (150 mm×25 mm×5 μm). Flow rate: 25 mL/min. Gradient: 1% to 33% CH₃CN in (10 mM NH₄HCO₃ in H₂O) (9 min) then 100% CH₃CN (2 min). The title compound (520 mg, 74.5% yield) was obtained as a yellow lyophilized solid. LC-MS: m/z=226.9 [M+H]⁺, ESI pos.

### Step 4: 1-[3-acetyl-6-[5-(6-methylpyridazin-3-yl)oxybenzimidazol-1-yl]-2-pyridyl]-5-methyl-pyrazole-3-carbonitrile and 1-[3-acetyl-6-[6-(6-methylpyridazin-3-yl)oxybenzimidazol-1-yl]-2-pyridyl]-5-methyl-pyrazole-3-carbonitrile

Error! Objects cannot be created from editing field codes.

Following the procedure described in step 2 of example 64 and with a reaction time of 3 hours. Purification by preparative HPLC: column Phenomenex Synergi C18 (150 mm×25 mm×10 μm). Flow rate: 25 mL/min. Gradient: 30% to 60% CH₃CN in (0.225% formic acid in H₂O v/v) (8.5 min) then 100% CH₃CN (2 min). Further purification by chiral SFC performed to separate the 2 regioisomers (Daicel Chiralpak AD (250 mm×30 mm×10 μm), flow rate: 80 mL/min. 70% (0.1% NH₄OH in MeOH) to yield 1-[3-acetyl-6-[5-(6-methylpyridazin-3-yl)oxybenzimidazol-1-yl]-2-pyridyl]-5-methyl-pyrazole-3-carbonitrile (38 mg, 11% yield) as a white lyophilized solid and 1-[3-acetyl-6-[6-(6-methylpyridazin-3-yl)oxybenzimidazol-1-yl]-2-pyridyl]-5-methyl-pyrazole-3-carbonitrile (44 mg, 12.7% yield) as white lyophilized solid. Regioisomer 1: LC-MS: m/z=451.1 [M+H]⁺, ESI pos. ¹H NMR (400 MHz, CD₃OD): δ=9.00 (s, 1H), 8.44 (d, J=8.4 Hz, 1H), 8.17-8.06 (m, 2H), 7.82 (d, J=8.7 Hz, 1H), 7.64 (d, J=9.0 Hz, 1H), 7.35 (d, J=9.0 Hz, 1H), 7.26 (dd, J=2.3, 8.7 Hz, 1H), 6.81 (s, 1H), 2.63 (s, 3H), 2.47 (s, 3H), 2.19 (s, 3H). Regioisomer 2: LC-MS: m/z=451.1 [M+H]⁺, ESI pos. ¹H NMR (400 MHz, CD₃OD): δ=9.01 (s, 1H), 8.46 (d, J=8.4 Hz, 1H), 8.25 (d, J=8.9 Hz, 1H), 8.13 (d, J=8.3 Hz, 1H), 7.64 (d, J=9.2 Hz, 1H), 7.58 (d, J=2.0 Hz, 1H), 7.35 (d, J=9.0 Hz, 1H), 7.26 (dd, J=1.7, 8.9 Hz, 1H), 6.88 (s, 1H), 2.61 (s, 3H), 2.57 (s, 3H), 2.22 (s, 3H).

### Step 5: 1-[3-(-hydroxyethyl)-6-[6-(6-methylpyridazin-3-yl)oxybenzimidazol-1-yl]-2-pyridyl]-5-methyl-pyrazole-3-carbonitrile

Error! Objects cannot be created from editing field codes.

To a solution of 1-[3-acetyl-6-[6-(6-methylpyridazin-3-yl)oxybenzimidazol-1-yl]-2-pyridyl]-5-methyl-pyrazole-3-carbonitrile (10 mg, 0.02 mmol, 1 equiv.) in Methanol (3 mL) was added NaBH₄ (2 mg, 0.05 mmol, 2.38 equiv.) at 0° C. The mixture was stirred at 0° C. for 0.5 h and directly purified by preparative HPLC: column Phenomenex Synergi C18 (150 mm×25 mm×10 μm). Flow rate: 25 mL/min. Gradient: 28% to 58% CH₃CN in (0.225% formic acid in H₂O v/v) (8.5 min) then 100% CH₃CN (2 min). The title compound (7.6 mg, 75.2% yield) was obtained as a white lyophilized solid. LC-MS: m/z=453.1 [M+H]⁺, ESI pos. ¹H NMR (400 MHz, CD₃OD): δ=9.00 (s, 1H), 8.51 (d, J=8.4 Hz, 1H), 8.16 (d, J=8.5 Hz, 1H), 8.04 (d, J=2.3 Hz, 1H), 7.84 (d, J=8.8 Hz, 1H), 7.66 (d, J=9.0 Hz, 1H), 7.37 (d, J=9.0 Hz, 1H), 7.27 (dd, J=2.3, 8.8 Hz, 1H), 6.80 (s, 1H), 4.81-4.73 (m, 2H), 2.66 (s, 3H), 2.32 (s, 3H), 1.40 (d, J=6.5 Hz, 3H).

### Example 75

### 1-[3-(1-Hydroxyethyl)-6-[5-(6-methylpyridazin-3-yl)oxybenzimidazol-1-yl]-2-pyridyl]-5-methyl-pyrazole-3-carbonitrile

Error! Objects cannot be created from editing field codes.

Starting from 1-[3-acetyl-6-[5-(6-methylpyridazin-3-yl)oxybenzimidazol-1-yl]-2-pyridyl]-5-methyl-pyrazole-3-carbonitrile (obtained as in step 4 of example 74) (38 mg, 0.08 mmol, 1 equiv.) and following the procedure described in step 5 of example 74, the title compound (25.3 mg, 66.3% yield) was obtained as a white lyophilized solid. Purification by preparative HPLC: column Phenomenex Synergi C18 (150 mm×25 mm×10 μm). Flow rate: 25 mL/min. Gradient: 28% to 58% CH₃CN in (0.225% formic acid in H₂O v/v) (8.5 min) then 100% CH₃CN (2 min). LC-MS: m/z=453.1 [M+H]⁺, ESI pos. ¹H NMR (400 MHz, CD₃OD): δ=9.00 (s, 1H), 8.53 (d, J=8.4 Hz, 1H), 8.28-8.21 (m, 1H), 8.18 (d, J=8.6 Hz, 1H), 7.64 (d, J=9.0 Hz, 1H), 7.59 (d, J=2.1 Hz, 1H), 7.35 (d, J=9.2 Hz, 1H), 7.27 (br d, J=8.3 Hz, 1H), 6.86 (s, 1H), 4.82-4.74 (m, 1H), 2.61 (s, 3H), 2.41 (s, 3H), 1.42 (d, J=6.5 Hz, 3H).

### Example 76

### 5-[3-(1-Hydroxyethyl)-6-[5-[(6-methylpyridazin-3-yl)amino]benzimidazol-1-yl]-2-pyridyl]-1-methyl-pyrazole-4-carbonitrile

Error! Objects cannot be created from editing field codes.

### Step 1: 1-(2-chloro-6-fluoro-3-pyridyl)ethanone

Error! Objects cannot be created from editing field codes.

A mixture of 3-bromo-2-chloro-6-fluoro-pyridine (5.0 g, 23.76 mmol, 1 equiv.), tributyl(1-ethoxyvinyl)tin (10.3 g, 28.51 mmol, 1.2 equiv.), K₂CO₃ (9.85 g, 71.28 mmol, 3 equiv.) and dichloropalladium triphenylphosphine (3.34 g, 4.75 mmol, 0.2 equiv.) in a mixture of 1,4-dioxane (80 mL) and H₂O (5 mL) was stirred at 100° C. for 12 hours under N₂ atmosphere. The reaction mixture was cooled to RT, poured into sat. aq. KF sol. (50 mL) and stirred for 2 hours. The mixture was then extracted with EtOAc (3×20 mL). The combined organic layers were treated with 2N HCl (20 mL) and the mixture was stirred for 2 hours. Then the mixture was basified by the addition of aqueous Na₂CO₃ until pH 8 was reached. The layers were separated. The aqueous phase was extracted with EtOAc (2×20 mL). The combined organic layers were dried over Na₂SO₄, filtered and concentrated under vacuum. The residue was purified by flash chromatography (SiO₂, 10% EtOAc in petroleum ether). The title compound (1.9 g, 46.1% yield) was obtained as a yellow oil. ¹H NMR (400 MHz, CDCl₃): δ=8.13 (dd, J=7.6, 8.3 Hz, 1H), 6.98 (dd, J=3.3, 8.4 Hz, 1H), 2.72 (s, 3H).

### Step 2: 1-[2-chloro-6-[5-[(6-methylpyridazin-3-yl)amino]benzimidazol-1-yl]-3-pyridyl]ethanone

Error! Objects cannot be created from editing field codes.

A solution of 1-(2-chloro-6-fluoro-3-pyridyl)ethanone (1.9 g, 10.95 mmol, 1 equiv.), N,N-diisopropylethylamine (3.8 mL, 21.89 mmol, 2 equiv.) and N-(6-methylpyridazin-3-yl)-1H-benzimidazol-5-amine (obtained as in intermediate 1 of example 64) (2.47 g, 10.95 mmol, 1 equiv.) in DMSO (30 mL) was stirred at 60° C. for 12 hours. The reaction mixture was cooled to rt, poured into H₂O (100 mL) and extracted with EtOAc (3×200 mL). The combined organic layers were dried over Na₂SO₄, filtered and concentrated under vacuum. The residue was purified by preparative NPLC: column Welch Ultimate XB-SiOH (250 mm×70 mm×10 μm). Flow rate: 140 mL/min. Gradient: 10% to 50% EtOH in hexane (25 min) then 100% EtOH (4 min). The title compound (500 mg, 1.32 mmol, 12.1% yield) was obtained as a brown solid. LC-MS: m/z=379.0 [M+H]⁺, ESI pos. ¹H NMR (400 MHz, DMSO-d₆): δ=9.30 (br s, 1H), 9.08 (s, 1H), 8.49-8.44 (m, 2H), 8.30 (d, J=8.9 Hz, 1H), 8.11 (s, 1H), 7.53 (dd, J=2.1, 8.9 Hz, 1H), 7.35 (d, J=9.1 Hz, 1H), 7.11 (d, J=9.1 Hz, 1H), 2.68 (s, 3H), 2.49 (br s, 3H).

### Step 3: 5-[3-acetyl-6-[5-[(6-methylpyridazin-3-yl)amino]benzimidazol-1-yl]-2-pyridyl]-1-methyl-pyrazole-4-carbonitrile

Error! Objects cannot be created from editing field codes.

To a solution of 1-[2-chloro-6-[5-[(6-methylpyridazin-3-yl)amino]benzimidazol-1-yl]-3-pyridyl]ethanone (100 mg, 0.26 mmol, 1 equiv.) in 1,4-dioxane (6 mL) and H₂O (0.3 mL) were added 1-methyl-5-(4,4,5,5-tetramethyl-1,3,2-dioxaborolan-2-yl)pyrazole-4-carbonitrile (92 mg, 0.4 mmol, 1.5 equiv.), Na₂CO₃ (84 mg, 0.79 mmol, 3 equiv.) and PdCl₂(dppf) CH₂Cl₂ (43 mg, 0.05 mmol, 0.200 equiv.). The brown reaction mixture was stirred at 100° C. for 8 h under N₂ atmosphere. The reaction mixture was cooled to rt, poured into H₂O (10 mL) and extracted with EtOAc (3×40 mL). The combined organic layers were dried over Na₂SO₄, filtered and concentrated under reduced pressure. The residue was purified by flash chromatography (silica gel, 10% MeOH in DCM). The title compound (20 mg, 16% yield) was obtained as a brown solid. LC-MS: m/z=450.3 [M+H]⁺, ESI pos.

### Step 4: 5-[3-(1-hydroxyethyl)-6-[5-[(6-methylpyridazin-3-yl)amino]benzimidazol-1-yl]-2-pyridyl]-1-methyl-pyrazole-4-carbonitrile

Error! Objects cannot be created from editing field codes.

Following the procedure described in example 75, with a reaction time of 1 h, the title compound (2.2 mg, 9.9% yield) was obtained as light yellow lyophilized solid. Purification by preparative HPLC: column Phenomenex Luna C18 (150 mm×25 mm×10 μm). Flow rate: 25 mL/min. Gradient: 2% to 32% CH₃CN in (0.225% formic acid in H₂O v/v) (10 min) then 100% CH₃CN (2 min). LC-MS: m/z=452.2 [M+H]⁺, ESI pos. ¹H NMR (400 MHz, CD₃OD): δ=8.90 (s, 1H), 8.43 (d, J=8.6 Hz, 1H), 8.34 (br s, 2H), 8.23 (s, 1H), 8.14 (br d, J=8.4 Hz, 2H), 8.09-8.02 (m, 1H), 7.58 (dd, J=1.9, 8.9 Hz, 1H), 7.36 (d, J=9.1 Hz, 1H), 7.14 (d, J=9.1 Hz, 1H), 3.86 (s, 3H), 2.53 (s, 3H), 1.59-1.41 (m, 3H).

### Example 77

### 1-[3-(1-Hydroxyethyl)-6-[5-[(3R)-3-hydroxypyrrolidin-1-yl]benzimidazol-1-yl]-2-pyridyl]-5-methyl-pyrazole-3-carbonitrile; formic acid

Error! Objects cannot be created from editing field codes.

### Step 1: 1-[6-(5-bromobenzimidazol-1-yl)-3-(1-hydroxyethyl)-2-pyridyl]-5-methyl-pyrazole-3-carbonitrile

Error! Objects cannot be created from editing field codes.

To a suspension of 1-[3-acetyl-6-(5-bromobenzimidazol-1-yl)-2-pyridyl]-5-methyl-pyrazole-3-carbonitrile (obtained as in step 1 of example 65) (750 mg, 1.78 mmol, 1 equiv.) in MeOH (10 mL) and THF (10 mL) was added NaBH₄ (202 mg, 5.34 mmol, 3 equiv.) at 0° C. The mixture was stirred at 0° C. for 1 hour. The reaction mixture was then diluted with H₂O (100 mL) and extracted with EtOAc (5×100 mL. The combined organics were washed with brine (2×100 mL), dried (Na₂SO₄), filtered and concentrated to give the crude title compound (750 mg, 99.5% yield) as an off-white solid. LC-MS: m/z=425.0 [M+H]⁺, ESI pos. ¹H NMR (400 MHz, CDCl₃): δ=8.50 (s, 1H), 8.32 (d, J=8.4 Hz, 1H), 7.96 (d, J=1.8 Hz, 1H), 7.82 (d, J=8.8 Hz, 1H), 7.69 (d, J=8.4 Hz, 1H), 7.44 (dd, J=1.8, 8.8 Hz, 1H), 6.64 (s, 1H), 4.81 (q, J=6.5 Hz, 1H), 2.42-2.39 (m, 3H), 1.45 (d, J=6.5 Hz, 3H).

### Step 2: 1-[3-(-hydroxyethyl)-6-[5-[(3R)-3-hydroxypyrrolidin-1-yl]benzimidazol-1-yl]-2-pyridyl]-5-methyl-pyrazole-3-carbonitrile; formic acid

Error! Objects cannot be created from editing field codes.

Following the procedure described in step 2 of example 71, with (R)-3-hydroxypyrrolidine (0.02 mL, 0.26 mmol, 1.1 equiv.), the title compound (17.3 mg, 16.2% yield) was obtained as yellow lyophilized solid. Purification by prep-HPLC: column Phenomenex Luna C18 (150 mm×25 mm×10 μm). Flow rate: 25 mL/min. Gradient: 15% to 45% CH₃CN in (0.225% formic acid in H₂O v/v) (10 min) then 100% CH₃CN (2 min). LC-MS: m/z=430.1 [M+H]⁺, ESI pos. ¹H NMR (400 MHz, CD₃OD): δ=8.80 (s, 1H), 8.48 (d, J=8.5 Hz, 1H), 8.11 (d, J=8.6 Hz, 1H), 8.02 (d, J=9.0 Hz, 1H), 6.89-6.85 (m, 2H), 6.80 (dd, J=2.0, 9.1 Hz, 1H), 4.77 (q, J=6.5 Hz, 1H), 4.61-4.54 (m, 1H), 3.62-3.49 (m, 2H), 3.41 (dt, J=3.8, 8.7 Hz, 1H), 3.28 (br d, J=10.4 Hz, 1H), 2.42 (s, 3H), 2.28-2.18 (m, 1H), 2.12-2.02 (m, 1H), 1.43 (d, J=6.5 Hz, 3H).

### Example 78

### 1-[3-(1-hydroxyethyl)-6-[5-[(3S)-3-hydroxypyrrolidin-1-yl]benzimidazol-1-yl]-2-pyridyl]-5-methyl-pyrazole-3-carbonitrile; formic acid

Error! Objects cannot be created from editing field codes.

Following the procedure described in step 2 of example 71, with (S)-3-hydroxypyrrolidine (0.02 mL, 0.260 mmol, 1.1 equiv.), the title compound (21.6 mg, 18.1% yield) was obtained as yellow lyophilized solid. Purification by prep-HPLC: column Phenomenex Luna C18 (150 mm×25 mm×10 μm). Flow rate: 25 mL/min. Gradient: 15% to 45% CH₃CN in (0.225% formic acid in H₂O v/v) (10 min) then 100% CH₃CN (2 min). LC-MS: m/z=430.1 [M+H]⁺, ESI pos. ¹H NMR (400 MHz, CD₃OD): δ=8.78 (s, 1H), 8.46 (d, J=8.4 Hz, 1H), 8.31 (s, 1H), 8.09 (d, J=8.6 Hz, 1H), 8.00 (d, J=9.0 Hz, 1H), 6.87-6.83 (m, 2H), 6.78 (dd, J=2.3, 9.1 Hz, 1H), 4.77-4.72 (m, 1H), 4.59-4.53 (m, 1H), 3.58-3.48 (m, 2H), 3.39 (dt, J=3.4, 8.6 Hz, 1H), 3.25 (br s, 1H), 2.40 (s, 3H), 2.27-2.14 (m, 1H), 2.11-1.99 (m, 1H), 1.41 (d, J=6.5 Hz, 3H).

### Example 81

### 1-[3-(1-Hydroxyethyl)-6-[5-[(6-methyl-3-pyridyl)amino]benzimidazol-1-yl]-2-pyridyl]-5-methyl-pyrazole-3-carbonitrile

Error! Objects cannot be created from editing field codes.

### Step 1: 1-[3-acetyl-6-[5-[(6-methyl-3-pyridyl)amino]benzimidazol-1-yl]-2-pyridyl]-5-methyl-pyrazole-3-carbonitrile

Error! Objects cannot be created from editing field codes.

Argon was bubbled for 5 min through a suspension of 1-[3-acetyl-6-(5-bromobenzimidazol-1-yl)-2-pyridyl]-5-methyl-pyrazole-3-carbonitrile (obtained as in step 1 of example 65) (37 mg, 0.087 mmol, 1 equiv.), (6-methyl-3-pyridyl)amine (19 mg, 0.175 mmol, 2 equiv.) and Cs₂CO₃ (85 mg, 0.262 mmol, 3 eq uiv.) at RT in 1,4-dioxane (1.2 mL). Then tBuXphos-Pd-G3 (7 mg, 0.009 mmol, 0.1 equiv.) was added, the vial was sealed, the mixture was then heated to 90° C. and stirring was continued for 4 hours. More tBuXphos-Pd-G3 (7 mg, 0.009 mmol, 0.1 equiv.) was added and the mixture was stirred for another 4 h at 90° C. The reaction mixture was cooled to RT, adsorbed on silica gel and directly purified by flash chromatography (12 g silica gel, 0% to 100% [DCM/MeOH 9:1] in DCM). The isolated product was purified again by flash chromatography (10 g Si—NH₂, 20% to 100% EtOAc in heptane). The title compound (21.7 mg, 46.9% yield) was obtained as a yellow solid. LC-MS: m/z=449.2 [M+H]⁺, ESI pos.

### Step 2: 1-[3-(-hydroxyethyl)-6-[5-[(6-methyl-3-pyridyl)amino]benzimidazol-1-yl]-2-pyridyl]-5-methyl-pyrazole-3-carbonitrile

Error! Objects cannot be created from editing field codes.

To a solution of 1-[3-acetyl-6-[5-[(6-methyl-3-pyridyl)amino]benzimidazol-1-yl]-2-pyridyl]-5-methyl-pyrazole-3-carbonitrile (22 mg, 0.041 mmol, 1 equiv.) in MeOH (1 mL) was added NaBH₄ (5 mg, 0.123 mmol, 3 equiv.) at 0° C. The mixture was stirred at 0° C. for 30 min. The reaction mixture was quenched at 0° C. with NH₄Cl aq. sat. sol. and extracted with DCM. The organic layer was dried over MgSO₄, filtered and concentrated in vacuo. The residue was adsorbed on silica gel and purified by flash chromatography (4 g silica gel, 0% to 100% [DCM/MeOH 9:1] in DCM). The title compound (11 mg, 58.8% yield) was obtained as a light yellow solid. LC-MS: m/z=451.3 [M+H]⁺, ESI pos.

### Example 82

### 1-[3-(1-Hydroxyethyl)-6-[5-[(5-methylpyrazin-2-yl)amino]benzimidazol-1-yl]-2-pyridyl]-5-methyl-pyrazole-3-carbonitrile

Error! Objects cannot be created from editing field codes.

### Step 1: 1-[3-acetyl-6-[5-[(5-methylpyrazin-2-yl)amino]benzimidazol-1-yl]-2-pyridyl]-5-methyl-pyrazole-3-carbonitrile

Error! Objects cannot be created from editing field codes.

Following the procedure described in step 1 of example 81, with (5-methylpyrazin-2-yl)amine (19 mg, 0.175 mmol, 2 equiv.), the title compound (12 mg, 29% yield) was obtained as a yellow solid. LC-MS: m/z=450.2 [M+H]⁺, ESI pos.

### Step 2: 1-[3-(1-hydroxyethyl)-6-[5-[(5-methylpyrazin-2-yl)amino]benzimidazol-1-yl]-2-pyridyl]-5-methyl-pyrazole-3-carbonitrile

Error! Objects cannot be created from editing field codes.

Following the procedure described in step 2 of example 81, the title compound (4.5 mg, 37.3% yield) was obtained as an off-white solid. LC-MS: m/z=452.3 [M+H]⁺, ESI pos.

### Example 83

### 1-[3-(1-Hydroxyethyl)-6-[5-[(5-methylpyrimidin-2-yl)amino]benzimidazol-1-yl]-2-pyridyl]-5-methyl-pyrazole-3-carbonitrile

Error! Objects cannot be created from editing field codes.

### Step 1: 1-[3-acetyl-6-[5-[(5-methylpyrimidin-2-yl)amino]benzimidazol-1-yl]-2-pyridyl]-5-methyl-pyrazole-3-carbonitrile

Error! Objects cannot be created from editing field codes.

Following the procedure described in step 1 of example 81, with (5-methylpyrimidin-2-yl)amine (27 mg, 0.237 mmol, 2 eq), the title compound (14.1 mg, 25.6% yield) was obtained as a light yellow solid. LC-MS: m/z=450.2 [M+H]⁺, ESI pos.

### Step 2: 1-[3-(-hydroxyethyl)-6-[5-[(5-methylpyrimidin-2-yl)amino]benzimidazol-1-yl]-2-pyridyl]-5-methyl-pyrazole-3-carbonitrile

Error! Objects cannot be created from editing field codes.

To a mixture of 1-[3-acetyl-6-[5-[(5-methylpyrimidin-2-yl)amino]benzimidazol-1-yl]-2-pyridyl]-5-methyl-pyrazole-3-carbonitrile (14 mg, 0.031 mmol, 1 eq) in MeOH (0.4 mL) was added NaBH₄ (3 mg, 0.076 mmol, 3 eq) at 0° C. The mixture was stirred at 0° C. for 15 min. Then THF (0.4 mL) was added to the mixture which turned to a clear solution. Stirring at 0° C. was continued for another 15 min. The reaction mixture was quenched at 0° C. with NH₄Cl aq. sat. sol. and extracted with DCM. The organic layer was dried over MgSO₄, filtered and concentrated in vacuo. The residue was adsorbed on silica gel and purified by flash chromatography (4 g silica gel, 0% to 100% [DCM/MeOH 9:1] in DCM). The title compound (9.2 mg, 63.7% yield) was obtained as a white solid. LC-MS: m/z=452.3 [M+H]⁺, ESI pos.

### Example 84

### 1-[3-(1-hydroxyethyl)-6-[5-[(5-methyl-2-pyridyl)amino]benzimidazol-1-yl]-2-pyridyl]-5-methyl-pyrazole-3-carbonitrile

Error! Objects cannot be created from editing field codes.

### Step 1: 1-[3-acetyl-6-[5-[(5-methyl-2-pyridyl)amino]benzimidazol-1-yl]-2-pyridyl]-5-methyl-pyrazole-3-carbonitrile

Error! Objects cannot be created from editing field codes.

Following the procedure described in step 1 of example 81, with (5-methyl-2-pyridyl)amine (26 mg, 0.237 mmol, 2 equiv.), the title compound (27.5 mg, 46.5% yield) was obtained as a yellow solid. LC-MS: m/z=449.2 [M+H]⁺, ESI pos.

### Step 2: 1-[3-(-hydroxyethyl)-6-[5-[(5-methyl-2-pyridyl)amino]benzimidazol-1-yl]-2-pyridyl]-5-methyl-pyrazole-3-carbonitrile

Error! Objects cannot be created from editing field codes.

To a mixture of 1-[3-acetyl-6-[5-[(5-methyl-2-pyridyl)amino]benzimidazol-1-yl]-2-pyridyl]-5-methyl-pyrazole-3-carbonitrile (27 mg, 0.061 mmol, 1 equiv.) in MeOH (0.8 mL) and THF, extra dry (0.8 mL) was added NaBH₄ (3 mg, 0.076 mmol, 3 equiv.) at 0° C. The mixture was stirred at 0° C. for 15 min. The reaction mixture was quenched at 0° C. with NH₄Cl aq. sat. sol. and extracted with DCM. The organic layer was dried over MgSO₄, filtered and concentrated in vacuo. The residue was adsorbed on silica gel and purified by flash chromatography (4 g silica gel, 0% to 100% [DCM/MeOH 9:1] in DCM). The title compound (21 mg, 72.2% yield) was obtained as a light yellow solid. LC-MS: m/z=451.4 [M+H]⁺, ESI pos.

### Example 85

### 1-[2-(1,1-dioxo-1,2-thiazolidin-2-yl)-6-[5-[(6-methylpyridazin-3-yl)amino]benzimidazol-1-yl]-3-pyridyl]ethanol

Error! Objects cannot be created from editing field codes.

### Step 1: 1-[6-chloro-2-(1,1-dioxo-1,2-thiazolidin-2-yl)-3-pyridyl]ethanone

Error! Objects cannot be created from editing field codes.

To a solution of 1-(6-chloro-2-fluoro-3-pyridyl)ethanone (CAS #1260663-13-5, 500 mg, 2.88 mmol, 1 equiv.) in DMSO (10 mL) were added isothiazolidine 1,1-dioxide (349 mg, 2.88 mmol, 1 equiv.) and added K₂CO₃ (1.19 g, 8.64 mmol, 3 equiv.). The mixture was stirred at 30° C. for 2 hours. The mixture poured into H₂O (30 mL) and extracted with EtOAc (3×15 mL). The combined organic extracts were washed with brine (3×15 mL), dried over anhydrous Na₂SO₄, filtered and concentrated under reduced pressure. The residue was purified by flash chromatography (silica gel, 0% to 100% EtOAc in petroleum ether). The title compound (480 mg, 60.6% yield) was obtained as a light red solid. LC-MS: m/z=274.8 [M+H]⁺, ESI pos.

### Step 2: 1-[2-(1,1-dioxo-1,2-thiazolidin-2-yl)-6-[5-[(6-methylpyridazin-3-yl)amino]benzimidazol-1-yl]-3-pyridyl]ethanone and 1-[2-(1,1-dioxo-1,2-thiazolidin-2-yl)-5-[6-[(6-methylpyridazin-3-yl)amino]benzimidazol-1-yl]-3-pyridyl]ethanone

Error! Objects cannot be created from editing field codes.

To a solution of 1-[6-chloro-2-(1,1-dioxo-1,2-thiazolidin-2-yl)-3-pyridyl]ethanone (460 mg, 1.67 mmol, 1 equiv.) in DMSO (10 mL) were added N-(6-methylpyridazin-3-yl)-1H-benzimidazol-5-amine (obtained as in intermediate 1 of example 64) (453 mg, 2.01 mmol, 1.2 equiv.) and K₂CO₃ (694 mg, 5.02 mmol, 3 equiv.). The brown suspension was stirred at 80° C. for 2 hours. The mixture was directly purified by preparative HPLC: column Waters Xbridge C18 (150 mm×50 mm×10 μm). Flow rate: 60 mL/min.

Gradient: 16% to 46% CH₃CN in (10 mM NH₄HCO₃ in H₂O) (11 min) then 100% CH₃CN (2 min). A 1:1 mixture of the 2 title compounds (150 mg) was isolated. Further purification by chiral SFC (Daicel Chiralpak AD (250 mm×30 mm×10 μm). Flow rate: 70 mL/min. 50% (1% NH₄OH in isopropanol) to yield 1-[2-(1,1-dioxo-1,2-thiazolidin-2-yl)-6-[5-[(6-methylpyridazin-3-yl)amino]benzimidazol-1-yl]-3-pyridyl]ethanone (55 mg, 7.1% yield) as a brown solid and 1-[2-(1,1-dioxo-1,2-thiazolidin-2-yl)-5-[6-[(6-methylpyridazin-3-yl)amino]benzimidazol-1-yl]-3-pyridyl]ethanone (35 mg, 4.5% yield) as a grey solid. Regioisomer 1: LC-MS: m/z=464.0 [M+H]⁺, ESI pos. ¹H NMR (400 MHz, CDCl₃): δ ppm 2.50 (s, 3H) 2.52-2.59 (m, 3H) 2.63 (s, 3H) 3.26 (t, J=7.50 Hz, 2H) 4.25 (t, J=6.82 Hz, 2H) 6.96 (d, J=9.13 Hz, 1H) 7.03-7.07 (m, 1H) 7.07-7.10 (m, 1H) 7.35 (d, J=8.38 Hz, 1H) 7.62 (d, J=8.63 Hz, 1H) 8.08 (d, J=8.25 Hz, 1H) 8.35 (s, 1H) 8.74 (d, J=1.75 Hz, 1H). Regioisomer 2: LC-MS: m/z=464.0 [M+H]⁺, ESI pos.

### Step 3: 1-[2-(1,1-dioxo-1,2-thiazolidin-2-yl)-6-[5-[(6-methylpyridazin-3-yl)amino]benzimidazol-1-yl]-3-pyridyl]ethanol

Error! Objects cannot be created from editing field codes.

To a solution of 1-[2-(1,1-dioxo-1,2-thiazolidin-2-yl)-6-[5-[(6-methylpyridazin-3-yl)amino]benzimidazol-1-yl]-3-pyridyl]ethanone (45 mg, 0.1 mmol, 1 equiv.) in MeOH (3 mL) was added NaBH₄ (112 mg, 0.29 mmol, 3 equiv.) at 0° C. The reaction mixture was stirred at 0° C. for 30 minutes. The mixture was quenched with saturated NH₄Cl (5 mL) at 0° C. and extracted with DCM (2×10 mL). The combined organics were dried over Na₂SO₄, filtered and concentracted under reduce pressure. The crude product was purified by preparative HPLC: column Phenomenex Gemini-NX (75 mm×30 mm×30 μm). Flow rate: 30 mL/min. Gradient: 10% to 40% CH₃CN in (10 mM NH₄HCO₃ in H₂O) (8 min) then 100% CH₃CN (2 min). The title compound (17.5 mg, 38.7% yield) was obtained as a white lyophilized solid. LC-MS: m/z=466.1 [M+H]⁺, ESI pos. ¹H NMR (400 MHz, CD₃OD): δ ppm 1.56 (d, J=6.36 Hz, 3H) 2.54-2.63 (m, 5H) 3.35-3.39 (m, 2H) 4.10-4.19 (m, 1H) 4.33 (dt, J=10.70, 7.06 Hz, 1H) 5.48-5.56 (m, 1H) 7.15 (d, J=9.17 Hz, 1H) 7.27-7.32 (m, 1H) 7.37 (d, J=9.05 Hz, 1H) 7.69 (d, J=8.68 Hz, 1H) 7.85 (d, J=8.31 Hz, 1H) 8.33 (d, J=8.44 Hz, 1H) 8.76 (s, 1H) 9.08 (d, J=1.83 Hz, 1H).

### Example 86

### 1-[3-(1-Hydroxyethyl)-6-[5-[(2-methylpyrimidin-5-yl)amino]benzimidazol-1-yl]-2-pyridyl]-5-methyl-pyrazole-3-carbonitrile

Error! Objects cannot be created from editing field codes.

### Step 1: 1-[3-acetyl-6-[5-[(2-methylpyrimidin-5-yl)amino]benzimidazol-1-yl]-2-pyridyl]-5-methyl-pyrazole-3-carbonitrile

Error! Objects cannot be created from editing field codes.

Following the procedure described in step 1 of example 81, with (2-methylpyrimidin-5-yl)amine (26 mg, 0.237 mmol, 2 equiv.), the title compound (41.5 mg, 70% yield) was obtained as a yellow solid. LC-MS: m/z=450.3 [M+H]⁺, ESI pos.

### Step 2: 1-[3-(1-hydroxyethyl)-6-[5-[(2-methylpyrimidin-5-yl)amino]benzimidazol-1-yl]-2-pyridyl]-5-methyl-pyrazole-3-carbonitrile

Error! Objects cannot be created from editing field codes.

Following the procedure described in step 2 of example 84, the title compound (24.7 mg, 62.5% yield) was obtained as an off-white solid. LC-MS: m/z=452.3 [M+H]⁺, ESI pos.

### Example 87

### 1-[3-(1-Hydroxyethyl)-6-[5-(pyridazin-4-ylamino)benzimidazol-1-yl]-2-pyridyl]-5-methyl-pyrazole-3-carbonitrile

Error! Objects cannot be created from editing field codes.

### Step 1: 1-[3-acetyl-6-[5-(pyridazin-4-ylamino)benzimidazol-1-yl]-2-pyridyl]-5-methyl-pyrazole-3-carbonitrile

Error! Objects cannot be created from editing field codes.

Following the procedure described in step 1 of example 81, with pyridazin-4-ylamine (23 mg, 0.237 mmol, 2 equiv.), the title compound (8.9 mg, 16.4% yield) was obtained as a yellow solid. LC-MS: m/z=436.3 [M+H]⁺, ESI pos.

### Step 2: 1-[3-(1-hydroxyethyl)-6-[5-(pyridazin-4-ylamino)benzimidazol-1-yl]-2-pyridyl]-5-methyl-pyrazole-3-carbonitrile

Error! Objects cannot be created from editing field codes.

Following the procedure described in step 2 of example 84, the title compound (4.1 mg, 43% yield) was obtained as a light yellow solid. LC-MS: m/z=438.2 [M+H]⁺, ESI pos.

### Example 88

### 1-[3-(1-Hydroxyethyl)-6-[5-[(6-methoxypyridazin-3-yl)amino]benzimidazol-1-yl]-2-pyridyl]-5-methyl-pyrazole-3-carbonitrile

Error! Objects cannot be created from editing field codes.

### Step 1: 1-[3-acetyl-6-[5-[(6-methoxypyridazin-3-yl)amino]benzimidazol-1-yl]-2-pyridyl]-5-methyl-pyrazole-3-carbonitrile

Error! Objects cannot be created from editing field codes.

Following the procedure described in step 1 of example 81, with (6-methoxypyridazin-3-yl)amine (30 mg, 0.237 mmol, 2 equiv.), the title compound (59.7 mg, 97.3% yield) was obtained as a yellow solid. LC-MS: m/z=466.3 [M+H]⁺, ESI pos.

### Step 2: 1-[3-(1-hydroxyethyl)-6-[5-[(6-methoxypyridazin-3-yl)amino]benzimidazol-1-yl]-2-pyridyl]-5-methyl-pyrazole-3-carbonitrile

Error! Objects cannot be created from editing field codes.

Following the procedure described in step 2 of example 84, the title compound (38.3 mg, 67.4% yield) was obtained as a light yellow solid. LC-MS: m/z=468.3 [M+H]⁺, ESI pos.

### Example 89

### 1-[3-(1-Hydroxyethyl)-6-[5-(pyridazin-3-ylamino)benzimidazol-1-yl]-2-pyridyl]-5-methyl-pyrazole-3-carbonitrile

Error! Objects cannot be created from editing field codes.

### Step 1: 1-[3-acetyl-6-[5-(pyridazin-3-ylamino)benzimidazol-1-yl]-2-pyridyl]-5-methyl-pyrazole-3-carbonitrile

Error! Objects cannot be created from editing field codes.

Following the procedure described in step 1 of example 81, with pyridazin-3-ylamine (23 mg, 0.237 mmol, 2 equiv.), the title compound (15.3 mg, 26.6% yield) was obtained as a yellow solid. LC-MS: m/z=436.3 [M+H]⁺, ESI pos.

### Step 2: 1-[3-(1-hydroxyethyl)-6-[5-(pyridazin-3-ylamino)benzimidazol-1-yl]-2-pyridyl]-5-methyl-pyrazole-3-carbonitrile

Error! Objects cannot be created from editing field codes.

Following the procedure described in step 2 of example 84, the title compound (9.1 mg, 65% yield) was obtained as a light yellow solid. LC-MS: m/z=438.3 [M+H]⁺, ESI pos.

### Example 90

### 1-[6-[5-[[6-(Difluoromethyl)pyridazin-3-yl]amino]benzimidazol-1-yl]-3-(1-hydroxyethyl)-2-pyridyl]-5-methyl-pyrazole-3-carbonitrile

Error! Objects cannot be created from editing field codes.

### Step 1: 1-[3-acetyl-6-[5-[[6-(difluoromethyl)pyridazin-3-yl]amino]benzimidazol-1-yl]-2-pyridyl]-5-methyl-pyrazole-3-carbonitrile

Error! Objects cannot be created from editing field codes.

Following the procedure described in step 1 of example 81, with [6-(difluoromethyl)pyridazin-3-yl]amine (35 mg, 0.237 mmol, 2 equiv.), the title compound (35.2 mg, 55% yield) was obtained as a light yellow solid. LC-MS: m/z=486.3 [M+H]⁺, ESI pos.

### Step 2: 1-[6-[5-[[6-(difluoromethyl)pyridazin-3-yl]amino]benzimidazol-1-yl]-3-(1-hydroxyethyl)-2-pyridyl]-5-methyl-pyrazole-3-carbonitrile

Error! Objects cannot be created from editing field codes.

Following the procedure described in step 2 of example 84, the title compound (15.4 mg, 47% yield) was obtained as a light yellow solid. LC-MS: m/z=488.2 [M+H]⁺, ESI pos.

### Example 93

### 1-[6-[6-Fluoro-5-[(6-methylpyridazin-3-yl)amino]benzimidazol-1-yl]-3-(1-hydroxyethyl)-2-pyridyl]-5-methyl-pyrazole-3-carbonitrile

Error! Objects cannot be created from editing field codes.

### Step 1: N-(2-fluoro-4,5-dinitro-phenyl)-6-methyl-pyridazin-3-amine

Error! Objects cannot be created from editing field codes.

To a solution of 1,2-difluoro-4,5-dinitro-benzene (1.0 g, 4.9 mmol, 1 equiv.) in DMSO (12 mL) were added 3-amino-6-methylpyridazine (535 mg, 4.9 mmol, 1 equiv.) and potassium tert-butoxide (1.1 g, 9.8 mmol, 2 equiv.). The brown suspension was stirred at 30° C. for 3 hours. The reaction mixture was poured into H₂O (100 mL). A solid precipitated out. The solid was collected by filtration and dried to give the crude title compound (600 mg, 41.8% yield) as a brown solid. LC-MS: m/z=294.0 [M+H]⁺, ESI pos.

### Step 2: 6-fluoro-N-(6-methylpyridazin-3-yl)-1H-benzimidazol-5-amine

Error! Objects cannot be created from editing field codes.

To a solution of N-(2-fluoro-4,5-dinitro-phenyl)-6-methyl-pyridazin-3-amine (2.5 g, 8.53 mmol, 1 equiv.) in formic acid (30 mL, 795 mmol, 93.27 equiv.) was added Nickel (333 mg, 5.68 mmol, 0.670 equiv.). The mixture was stirred at 30° C. for 16 h under H₂ (45 psi). The mixture was filtered and the filtrate was concentrated in vacuo. The residue was poured into MeOH (30 mL). A solid precipitated out. The solid was collected by filtration, washed with MeOH and dried to give a first crop of the title compound (480 mg). The filtrate was concentrated and the residue was purified by preparative HPLC: column Phenomenex Luna C18 (250 mm×70 mm×10 μm). Flow rate: 80 mL/min. Gradient: 1% to 30% CH₃CN in (0.225% formic acid in H₂O v/v) (20 min) then 100% CH₃CN (5 min). A second crop of the title compound (860 mg) was obtained. The title compound (1.34 g, 64.6% yield) was obtained as an off-white solid. LC-MS: m/z=244.1 [M+H]⁺, ESI pos. ¹H NMR (400 MHz, DMSO-d₆): δ=12.70-12.32 (m, 1H), 8.73 (br s, 1H), 8.51-8.37 (m, 1H), 8.20-8.09 (m, 2H), 7.49 (br d, J=11.4 Hz, 1H), 7.33 (d, J=9.2 Hz, 1H), 7.24-7.14 (m, 1H), 2.47 (s, 3H).

### Step 3: 1-[3-acetyl-6-[6-fluoro-5-[(6-methylpyridazin-3-yl)amino]benzimidazol-1-yl]-2-pyridyl]-5-methyl-pyrazole-3-carbonitrile and 1-[3-acetyl-6-[5-fluoro-6-[(6-methylpyridazin-3-yl)amino]benzimidazol-1-yl]-2-pyridyl]-5-methyl-pyrazole-3-carbonitrile

Error! Objects cannot be created from editing field codes.

To a solution of 6-fluoro-N-(6-methylpyridazin-3-yl)-1H-benzimidazol-5-amine (930 mg, 3.82 mmol, 1 equiv.) in DMSO (20 mL) were added 1-(3-acetyl-6-chloro-2-pyridyl)-5-methyl-pyrazole-3-carbonitrile (obtained as in step 1 of example 64) (1.2 g, 4.59 mmol, 1.2 equiv.) and K₂CO₃ (1.59 g, 11.47 mmol, 3 equiv.). The reaction mixture was stirred at 30° C. for 2 hours. The mixture was then poured into H₂O (100 mL) and extracted with EtOAc (3×50 mL). The combined organic extracts were washed with brine (3×15 mL), dried over anhydrous Na₂SO₄, filtered and concentrated. The residue was purified by preparative NPLC: column Welch Ultimate-XB-SiOH (250 mm×70 mm×10 μm). Flow rate: 140 mL/min. Gradient: 20% to 60% EtOH in hexane (20 min) then 100% EtOH (4 min) to yield 1-[3-acetyl-6-[6-fluoro-5-[(6-methylpyridazin-3-yl)amino]benzimidazol-1-yl]-2-pyridyl]-5-methyl-pyrazole-3-carbonitrile (200 mg, 11.2% yield) as a yellow solid and 1-[3-acetyl-6-[5-fluoro-6-[(6-methylpyridazin-3-yl)amino]benzimidazol-1-yl]-2-pyridyl]-5-methyl-pyrazole-3-carbonitrile (500 mg, 28% yield) as a yellow solid. Regioisomer 1: LC-MS: m/z=468.1 [M+H]⁺, ESI pos. ¹H NMR (400 MHz, DMSO-d₆): δ=9.17 (s, 1H), 8.90 (s, 1H), 8.72 (d, J=7.7 Hz, 1H), 8.57 (d, J=8.4 Hz, 1H), 8.34 (d, J=8.6 Hz, 1H), 8.08 (d, J=11.6 Hz, 1H), 7.41-7.35 (m, 1H), 7.29-7.23 (m, 1H), 7.16 (s, 1H), 2.54 (s, 3H), 2.49 (br s, 3H), 2.23 (s, 3H). Regioisomer 2: LC-MS: m/z=468.1 [M+H]⁺, ESI pos. ¹H NMR (400 MHz, CD₃OD): δ=9.17 (d, J=7.4 Hz, 1H), 8.88 (s, 1H), 8.48 (d, J=8.4 Hz, 1H), 8.09 (d, J=8.4 Hz, 1H), 7.52 (d, J=11.0 Hz, 1H), 7.39 (d, J=9.1 Hz, 1H), 7.25 (d, J=9.1 Hz, 1H), 6.83 (s, 1H), 2.58 (s, 3H), 2.56 (s, 3H), 2.23 (s, 3H).

### Step 6: 1-[6-[6-fluoro-5-[(6-methylpyridazin-3-yl)amino]benzimidazol-1-yl]-3-(1-hydroxyethyl)-2-pyridyl]-5-methyl-pyrazole-3-carbonitrile

Error! Objects cannot be created from editing field codes.

To a solution of 1-[3-acetyl-6-[6-fluoro-5-[(6-methylpyridazin-3-yl)amino]benzimidazol-1-yl]-2-pyridyl]-5-methyl-pyrazole-3-carbonitrile (200 mg, 0.430 mmol, 1 equiv.) in MeOH (7 mL) and DCM (3 mL) was added NaBH₄ (49 mg, 1.28 mmol, 3 equiv.) at 0° C. The reaction mixture was stirred at 0° C. for 30 minutes. The mixture was quenched with saturated NH₄Cl (5 mL) at 0° C. and extracted with DCM (2×10 mL). The combined organic layers were dried over Na₂SO₄, filtered and concentracted under reduce pressure. The residue was purified by preparative HPLC: column Phenomenex Synergi C18 (150 mm×25 mm×10 μm). Flow rate: 25 mL/min. Gradient: 8% to 38% CH₃CN in (0.225% formic acid in H₂O v/v) (20 min) then 100% CH₃CN (3 min). The title compound (120 mg, 59.7% yield) was obtained as a white lyophilized solid. LC-MS: m/z=470.2 [M+H]⁺, ESI pos. ¹H NMR (400 MHz, CD₃OD): δ=9.00-8.89 (m, 1H), 8.61-8.46 (m, 2H), 8.24-8.12 (m, 1H), 8.07-7.95 (m, 1H), 7.41 (d, J=9.1 Hz, 1H), 7.24 (d, J=9.3 Hz, 1H), 6.90 (s, 1H), 4.78 (q, J=6.5 Hz, 1H), 4.61 (br s, 1H), 2.56 (s, 3H), 2.43 (s, 3H), 1.43 (d, J=6.5 Hz, 3H).

### Example 94

### 1-[6-[5-Fluoro-6-[(6-methylpyridazin-3-yl)amino]benzimidazol-1-yl]-3-(1-hydroxyethyl)-2-pyridyl]-5-methyl-pyrazole-3-carbonitrile

Error! Objects cannot be created from editing field codes.

Following the procedure described in step 6 of example 93, using MeOH (3 mL) as solvent, the title compound (11.6 mg, 23.1% yield) was obtained as a white lyophilized solid. Purification by preparative HPLC (Phenomenex Gemini NX C18 (75 mm×30 mm×3 μm). Flow rate: 30 mL/min. Gradient: 18% to 48% CH₃CN in (10 mM NH₄HCO₃ in H2O) (8 min) then 100% CH₃CN (2 min). LC-MS: m/z=470.2 [M+H]⁺, ESI pos. ¹H NMR (400 MHz, CD₃OD): δ ppm 1.43 (d, J=6.48 Hz, 3H) 2.35-2.40 (m, 3H) 2.55-2.61 (m, 3H) 4.72-4.84 (m, 1H) 6.78-6.84 (m, 1H) 7.20-7.27 (m, 1H) 7.34-7.42 (m, 1H) 7.52-7.62 (m, 1H) 8.12-8.23 (m, 1H) 8.51-8.63 (m, 1H) 8.88 (s, 1H) 9.00-9.09 (m, 1H).

### Example 97

### 1-[6-[5-[(6-Methylpyridazin-3-yl)amino]benzimidazol-1-yl]-2-[3-(trifluoromethyl)-4,5,6,7-tetrahydropyrazolo[4,3-c]pyridin-1-yl]-3-pyridyl]ethanol

Error! Objects cannot be created from editing field codes.

### Step 1: tert-butyl 1-(3-acetyl-6-chloro-2-pyridyl)-3-(trifluoromethyl)-6,7-dihydro-4H-pyrazolo[4,3-c]pyridine-5-carboxylate

Error! Objects cannot be created from editing field codes.

To a solution of 1-(6-chloro-2-fluoro-3-pyridyl)ethanone (CAS #1260663-13-5, 500 mg, 2.88 mmol, 1 equiv.) in DMSO (8 mL) were added DIPEA (745 mg, 5.76 mmol, 2 equiv.) and tert-butyl 3-(trifluoromethyl)-1,4,6,7-tetrahydropyrazolo[4,3-c]pyridine-5-carboxylate (839 mg, 2.88 mmol, 1 equiv.). The reaction mixture was stirred at 80° C. for 7 hours. The mixture was cooled to rt, poured into H₂O (50 mL) and extracted with EtOAc (3×50 mL). The combined organic layers were concentracted and the residue was purified by flash chromatography (silica gel, 20% EtOAc in petroleum ether). The title (1.11 g, 86.6% yield) was obtained as a light yellow oil. LC-MS: m/z=389.0 [M+H]⁺, ESI pos. ¹H NMR (400 MHz, CDCl₃): δ=7.83 (d, J=8.0 Hz, 1H), 7.38 (d, J=8.0 Hz, 1H), 4.57 (br s, 2H), 3.77 (br t, J=5.3 Hz, 2H), 3.23 (br t, J=5.5 Hz, 2H), 2.31 (s, 3H), 1.51 (s, 9H).

### Step 2: tert-butyl 1-[3-acetyl-6-[5-[(6-methylpyridazin-3-yl)amino]benzimidazol-1-yl]-2-pyridyl]-3-(trifluoromethyl)-6,7-dihydro-4H-pyrazolo[4,3-c]pyridine-5-carboxylate and tert-butyl 1-[3-acetyl-6-[6-[(6-methylpyridazin-3-yl)amino]benzimidazol-1-yl]-2-pyridyl]-3-(trifluoromethyl)-6,7-dihydro-4H-pyrazolo[4,3-c]pyridine-5-carboxylate

Error! Objects cannot be created from editing field codes.

Following the procedure described in step 3 of example 76, with a reaction time of 72 h at 50° C., the crude material was purified by preparative NPLC: column Welch Ultimate XB-CN (250 mm×70 mm×10 μm). Flow rate: 140 mL/min. Gradient: 35% to 75% (0.1% NH₄OH in EtOH v/v) in heptane (15 min), then 0.1% NH₄OH in EtOH (3 min) to yield tert-butyl 1-[3-acetyl-6-[5-[(6-methylpyridazin-3-yl)amino]benzimidazol-1-yl]-2-pyridyl]-3-(trifluoromethyl)-6,7-dihydro-4H-pyrazolo[4,3-c]pyridine-5-carboxylate (1.5 g, 40.1% yield) as a brown solid and tert-butyl 1-[3-acetyl-6-[6-[(6-methylpyridazin-3-yl)amino]benzimidazol-1-yl]-2-pyridyl]-3-(trifluoromethyl)-6,7-dihydro-4H-pyrazolo[4,3-c]pyridine-5-carboxylate (1.4 g, 38.2% yield) as a brown solid. Regioisomer 1: LC-MS: m/z=634.3 [M+H]⁺, ESI pos. ¹H NMR (400 MHz, CD₃OD): δ=8.90 (s, 1H), 8.34 (d, J=8.3 Hz, 1H), 8.30 (d, J=1.4 Hz, 1H), 8.09 (d, J=8.9 Hz, 1H), 8.01 (d, J=8.3 Hz, 1H), 7.63 (br d, J=8.4 Hz, 1H), 7.38 (d, J=9.1 Hz, 1H), 7.15 (d, J=9.1 Hz, 1H), 4.63 (s, 2H), 3.79 (br t, J=5.7 Hz, 2H), 3.21 (br t, J=5.4 Hz, 2H), 2.55 (s, 3H), 2.27 (s, 3H), 1.52 (s, 9H). Regioisomer 2: LC-MS: m/z=634.3 [M+H]⁺, ESI pos. ¹H NMR (400 MHz, CD₃OD): δ=8.94 (s, 1H), 8.59 (s, 1H), 8.16 (d, J=8.2 Hz, 1H), 7.78 (d, J=8.2 Hz, 1H), 7.50 (d, J=8.8 Hz, 1H), 7.24-7.18 (m, 2H), 6.97 (d, J=9.0 Hz, 1H), 4.42 (s, 2H), 3.19 (d, J=1.1 Hz, 2H), 3.14-3.06 (m, 2H), 2.40 (s, 3H), 2.14 (s, 3H), 1.31 (br s, 9H).

### Step 3: 1-[6-[5-[(6-methylpyridazin-3-yl)amino]benzimidazol-1-yl]-2-[3-(trifluoromethyl)-4,5,6,7-tetrahydropyrazolo[4,3-c]pyridin-1-yl]-3-pyridyl]ethanone; hydrochloride

Error! Objects cannot be created from editing field codes.

Following the procedure described in step 2 of example 72, the crude title compound (1.2 g, 95% yield) was obtained as a light brown solid. LC-MS: m/z=534.2 [M+H]⁺, ESI pos.

### Step 4: 1-[6-[5-[(6-methylpyridazin-3-yl)amino]benzimidazol-1-yl]-2-[3-(trifluoromethyl)-4,5,6,7-tetrahydropyrazolo[4,3-c]pyridin-1-yl]-3-pyridyl]ethanol

Error! Objects cannot be created from editing field codes.

Following the procedure described in step 6 of example 93, using MeOH (4 mL) as solvent, the title compound (33.2 mg, 36.3% yield) was obtained as a white lyophilized solid. Purification by preparative HPLC: column Waters Xbridge (150 mm×25 mm×5 μm). Flow rate: 25 mL/min. Gradient: 21% to 51% CH₃CN in (10 mM NH₄HCO₃ in H2O) (10 min) then 100% CH₃CN (2 min). LC-MS: m/z=536.2 [M+H]⁺, ESI pos. ¹H NMR (400 MHz, CD₃OD): δ=8.87 (s, 1H), 8.52 (d, J=8.4 Hz, 1H), 8.19 (d, J=1.7 Hz, 1H), 8.09 (dd, J=8.6, 14.5 Hz, 2H), 7.70-7.62 (m, 1H), 7.36 (d, J=9.0 Hz, 1H), 7.13 (d, J=9.2 Hz, 1H), 5.07 (q, J=6.3 Hz, 1H), 4.14 (br s, 2H), 3.28-3.15 (m, 2H), 3.14-3.04 (m, 1H), 3.02-2.93 (m, 1H), 2.53 (s, 3H), 1.47 (d, J=6.5 Hz, 3H).

### Example 98

### 1-[6-[6-[(6-Methylpyridazin-3-yl)amino]benzimidazol-1-yl]-2-[3-(trifluoromethyl)-4,5,6,7-tetrahydropyrazolo[4,3-c]pyridin-1-yl]-3-pyridyl]ethanol

Error! Objects cannot be created from editing field codes.

### Step 1: 1-[6-[6-[(6-methylpyridazin-3-yl)amino]benzimidazol-1-yl]-2-[3-(trifluoromethyl)-4,5,6,7-tetrahydropyrazolo[4,3-c]pyridin-1-yl]-3-pyridyl]ethanone; hydrochloride

Error! Objects cannot be created from editing field codes.

Following the procedure described in step 2 of example 72, using tert-butyl 1-[3-acetyl-6-[6-[(6-methylpyridazin-3-yl)amino]benzimidazol-1-yl]-2-pyridyl]-3-(trifluoromethyl)-6,7-dihydro-4H-pyrazolo[4,3-c]pyridine-5-carboxylate (obtained as in step 2 of example 97) (1.4 g, 2.20 mmol, 1 equiv.), the crude title compound (1.12 g, 95% yield) was obtained as a light brown solid. LC-MS: m/z=534.2 [M+H]⁺, ESI pos.

### Step 2: 1-[6-[6-[(6-methylpyridazin-3-yl)amino]benzimidazol-1-yl]-2-[3-(trifluoromethyl)-4,5,6,7-tetrahydropyrazolo[4,3-c]pyridin-1-yl]-3-pyridyl]ethanol

Error! Objects cannot be created from editing field codes.

Following the procedure described in step 6 of example 93, using MeOH (4 mL) as solvent, the title compound (10.2 mg, 10.6% yield) was obtained as a yellow lyophilized solid. Purification by preparative HPLC: column Phenomenex Synergi C18 (150 mm×25 mm×10 μm). Flow rate: 25 mL/min. Gradient: 2% to 32% CH₃CN in (0.225% formic acid in H₂O v/v) (8.5 min) then 100% CH₃CN (2 min). LC-MS: m/z=536.2 [M+H]⁺, ESI pos. ¹H NMR (400 MHz, CD₃OD): δ=8.92-8.83 (m, 2H), 8.54 (d, J=8.6 Hz, 1H), 8.50-8.44 (m, 1H), 8.06 (d, J=8.4 Hz, 1H), 7.74-7.66 (m, 1H), 7.40 (d, J=9.0 Hz, 1H), 7.23 (dd, J=1.9, 8.5 Hz, 1H), 7.18-7.11 (m, 1H), 5.26 (q, J=6.1 Hz, 1H), 4.43 (br s, 2H), 3.85-3.68 (m, 1H), 3.55-3.46 (m, 1H), 3.45-3.36 (m, 2H), 2.56 (s, 3H), 1.51 (d, J=6.5 Hz, 3H).

### Example 103

### 1-[3-(1-Hydroxyethyl)-6-[5-[(5-methyl-1,3,4-oxadiazol-2-yl)amino]benzimidazol-1-yl]-2-pyridyl]-5-methyl-pyrazole-3-carbonitrile

Error! Objects cannot be created from editing field codes.

### Step 1: 1-[3-acetyl-6-[5-[(5-methyl-1,3,4-oxadiazol-2-yl)amino]benzimidazol-1-yl]-2-pyridyl]-5-methyl-pyrazole-3-carbonitrile

Error! Objects cannot be created from editing field codes.

According to the procedure described in step 1 of example 81, with (5-methyl-1,3,4-oxadiazol-2-yl)amine (25 mg, 0.237 mmol, 2 equiv.), the title compound (15.4 mg, 28.6% yield) was obtained as a light yellow solid. LC-MS: m/z=440.3 [M+H]⁺, ESI pos.

### Step 2: 1-[3-(-hydroxyethyl)-6-[5-[(5-methyl-1,3,4-oxadiazol-2-yl)amino]benzimidazol-1-yl]-2-pyridyl]-5-methyl-pyrazole-3-carbonitrile

Error! Objects cannot be created from editing field codes.

Following the procedure described in step 2 of example 84, the title compound (8.3 mg, 52.5% yield) was obtained as a white solid. LC-MS: m/z=442.2 [M+H]⁺, ESI pos.

### Example 104

### 2-[3-(1-Hydroxyethyl)-6-[5-[(6-methylpyridazin-3-yl)amino]benzimidazol-1-yl]-2-pyridyl]-5-methyl-pyrazole-3-carbonitrile

Error! Objects cannot be created from editing field codes.

### Step 1: 2-(3-acetyl-6-chloro-2-pyridyl)-5-methyl-pyrazole-3-carbonitrile

Error! Objects cannot be created from editing field codes.

1-(3-acetyl-6-chloro-2-pyridyl)-5-methyl-pyrazole-3-carbonitrile (obtained in step 1 of example 64) (6.03 g, 23.12 mmol) was purified by chiral SFC (Chiral IG (250 mm×30 mm×5 μm). 13% MeOH). The title compound (747 mg, 11.1% yield) was obtained as a white solid. LC-MS: m/z=261.1 [M+H]⁺, ESI pos.; ¹H NMR (600 MHz, CDCl₃): δ=7.83 (d, J=8.0 Hz, 1H), 7.42 (d, J=8.0 Hz, 1H), 6.89 (s, 1H), 2.35 (s, 3H), 2.28 (s, 3H).

### Step 2: 2-[3-acetyl-6-(5-bromobenzimidazol-1-yl)-2-pyridyl]-5-methyl-pyrazole-3-carbonitrile and 2-[3-acetyl-6-(6-bromobenzimidazol-1-yl)-2-pyridyl]-5-methyl-pyrazole-3-carbonitrile

Error! Objects cannot be created from editing field codes.

To a solution of 2-(3-acetyl-6-chloro-2-pyridyl)-5-methyl-pyrazole-3-carbonitrile (459 mg, 1.76 mmol, 1 equiv.) and 5-bromo-1H-benzimidazole (354 mg, 1.76 mmol, 1.0 equiv.) in DMSO (9 mL) was added 1,8-diazabicyclo[5.4.0]undec-7-ene (402 mg, 398 μL, 2.64 mmol, 1.5 equiv.). The reaction mixture was stirred at 23° C. for 24 hours. The mixture was poured into H₂O and extracted with EtOAc. The organic layer was dried over MgSO₄, filtered and concentrated in vacuo. The crude material was purified by flash chromatogaphy (25 g silica gel, 0% to 100% [DCM:MeOH 9:1] in DCM). Further purification by chiral SFC was required for the separation of the regioisomers (Chiral OZ-H (250 mm×20 mm×5 μm). 50% (0.2% diethylamine) in MeOH) to give 2-[3-acetyl-6-(5-bromobenzimidazol-1-yl)-2-pyridyl]-5-methyl-pyrazole-3-carbonitrile (183.5 mg, 24.4% yield) as an off-white solid. and 2-[3-acetyl-6-(6-bromobenzimidazol-1-yl)-2-pyridyl]-5-methyl-pyrazole-3-carbonitrile (129 mg, 17.1% yield) as an off-white solid. Regioisomer 1: LC-MS: m/z=421.0 [M+H]⁺, ESI pos. ¹H NMR (600 MHz, DMSO-d₆): δ=9.20-9.09 (m, 1H), 8.40 (d, J=8.4 Hz, 1H), 8.31 (d, J=8.8 Hz, 1H), 8.19 (d, J=8.4 Hz, 1H), 8.04 (d, J=2.0 Hz, 1H), 7.58 (dd, J=2.0, 8.7 Hz, 1H), 7.48 (s, 1H), 2.33 (s, 3H), 2.30 (s, 3H) Regioisomer 2: LC-MS: m/z=421.0 [M+H]⁺, ESI pos. ¹H NMR (600 MHz, DMSO-d₆): δ=9.15 (s, 1H), 8.60 (d, J=1.9 Hz, 1H), 8.48-8.46 (m, 1H), 8.40 (d, J=8.3 Hz, 1H), 8.36-8.32 (m, 1H), 8.23 (d, J=8.4 Hz, 1H), 7.93-7.84 (m, 1H), 7.77 (d, J=8.6 Hz, 1H), 7.55 (dd, J=1.9, 8.6 Hz, 1H), 7.50 (s, 1H), 2.44-2.44 (m, 1H), 2.33-2.32 (m, 1H), 2.33 (s, 2H), 2.31 (s, 3H).

### Step 3: 2-[3-acetyl-6-[5-[(6-methylpyridazin-3-yl)amino]benzimidazol-1-yl]-2-pyridyl]-5-methyl-pyrazole-3-carbonitrile

Error! Objects cannot be created from editing field codes.

Argon was bubbled for 5 min through a suspension of 2-[3-acetyl-6-(5-bromobenzimidazol-1-yl)-2-pyridyl]-5-methyl-pyrazole-3-carbonitrile (50 mg, 0.119 mmol, 1.0 equiv.), (6-methylpyridazin-3-yl)amine (26 mg, 0.237 mmol, 2 equiv.) and Cs₂CO₃ (116 mg, 0.356 mmol, 3.0 equiv.) at RT in 1,4-dioxane (1.2 mL). Then [tBuBrettPhos Pd(allyl)]OTf (17 mg, 0.024 mmol, 0.2 equiv.) was added, the vial was sealed, the mixture was then heated to 80° C. and stirring was continued for 2 hours. The reaction mixture was cooled to RT, adsorbed on silica gel and directly purified by flash chromatography (12 g silica gel, 0% to 100% [DCM/MeOH 9:1] in DCM). The title compound (36.8 mg, 65.5% yield) was obtained as a yellow solid. LC-MS: m/z=450.3 [M+H]⁺, ESI pos.

### Step 4: 2-[3-(1-hydroxyethyl)-6-[5-[(6-methylpyridazin-3-yl)amino]benzimidazol-1-yl]-2-pyridyl]-5-methyl-pyrazole-3-carbonitrile

Error! Objects cannot be created from editing field codes.

Following the procedure described in step 2 of example 84, the title compound (11.3 mg, 30.2% yield) was obtained as an off-white solid. LC-MS: m/z=452.3 [M+H]⁺, ESI pos.

### Example 105

### (3R)-1-[3-(1-Hydroxyethyl)-6-[5-[(6-methylpyridazin-3-yl)amino]benzimidazol-1-yl]-2-pyridyl]pyrrolidine-3-carbonitrile

Error! Objects cannot be created from editing field codes.

### Step 1: (3R)-1-[3-(1-hydroxyethyl)-6-[5-[(6-methylpyridazin-3-yl)amino]benzimidazol-1-yl]-2-pyridyl]pyrrolidine-3-carbonitrile

Error! Objects cannot be created from editing field codes.

To a solution of 1-(6-chloro-2-fluoro-3-pyridyl)ethanone (CAS #1260663-13-5, 400 mg, 2.3 mmol, 1 equiv.) and (R)-pyrrolidine-3-carbonitrile hydrochloride (336 mg, 2.54 mmol, 1.1 equiv.) in DMSO (6 mL) was added DIPEA (1.14 mL, 6.91 mmol, 3 equiv.). the reaction mixture was stirred at 30° C. for 3 hours. The mixture was poured into H₂O (50 mL) and extracted with EtOAc (3×50 mL×3). The combined organic layers were washed with brine(100 mL) and concentrated under vacuum. The residue was purified by flash chromatography (silica gel, 20% to 50% EtOAc in petroleum ether). The title compound (680 mg, quantitative yield) was obtained as a light green oil. LC-MS: m/z=250.1 [M+H]⁺, ESI pos.

### Step 2: (3R)-1-[3-acetyl-6-[5-[(6-methylpyridazin-3-yl)amino]benzimidazol-1-yl]-2-pyridyl]pyrrolidine-3-carbonitrile and (3R)-1-[3-acetyl-6-[6-[(6-methylpyridazin-3-yl)amino]benzimidazol-1-yl]-2-pyridyl]pyrrolidine-3-carbonitrile

Error! Objects cannot be created from editing field codes.

To a solution of (3R)-1-(3-acetyl-6-chloro-2-pyridyl)pyrrolidine-3-carbonitrile (460 mg, 1.84 mmol, 1 equiv.) in DMSO (9 mL) were added N-(6-methylpyridazin-3-yl)-1H-benzimidazol-5-amine (obtained as in intermediate 1 of example 64) (498 mg, 2.21 mmol, 1.2 equiv.) and K₂CO₃ (764 mg, 5.53 mmol, 3 equiv.). The brown suspension was then stirred at 80° C. for 16 hours. The mixture was cooled to RT, poured into H₂O (80 mL) and extracted with EtOAc (3×80 mL). The combined organic layers were washed with brine, dried over Na₂SO₄, filtered and concentrated in vacuo. The residue was purified by preparative NPLC (Welch Ultimate XB-CN (250 mm×70 mm×10 um). Flow rate 140 mL/min. Gradient: 55% to 95% EtOH in hexane (12 min) then 100% EtOH (5 min)) to give (3R)-1-[3-acetyl-6-[5-[(6-methylpyridazin-3-yl)amino]benzimidazol-1-yl]-2-pyridyl]pyrrolidine-3-carbonitrile (250 mg, 30.9% yield) as a yellow solid and (3R)-1-[3-acetyl-6-[6-[(6-methylpyridazin-3-yl)amino]benzimidazol-1-yl]-2-pyridyl]pyrrolidine-3-carbonitrile (250 mg, 30.9% yield) as a yellow solid. Regioisomer 1: LC-MS: m/z=439.1 [M+H]⁺, ESI pos. ¹H NMR (400 MHz, DMSO-d₆): δ=9.20 (s, 1H), 9.01 (s, 1H), 8.36-8.24 (m, 3H), 7.61 (dd, J=2.0, 8.9 Hz, 1H), 7.37-7.26 (m, 2H), 7.09 (d, J=9.0 Hz, 1H), 3.72-3.39 (m, 7H), 2.62 (s, 3H), 2.48 (br s, 3H). Regioisomer 2: LC-MS: m/z=439.1 [M+H]⁺, ESI pos. ¹H NMR (400 MHz, DMSO-d₆): δ=9.54 (d, J=1.7 Hz, 1H), 9.31 (s, 1H), 8.90 (s, 1H), 8.31 (d, J=8.3 Hz, 1H), 7.65 (d, J=8.7 Hz, 1H), 7.35 (d, J=9.0 Hz, 1H), 7.25 (d, J=8.3 Hz, 1H), 7.19 (dd, J=2.0, 8.7 Hz, 1H), 7.11 (d, J=9.0 Hz, 1H), 3.97 (br dd, J=6.9, 11.6 Hz, 1H), 3.70 (br dd, J=5.6, 11.6 Hz, 1H), 3.60-3.42 (m, 4H), 2.62 (s, 3H), 2.52 (s, 3H), 1.05 (t, J=7.0 Hz, 1H).

### Step 3: (3R)-1-[3-(1-hydroxyethyl)-6-[5-[(6-methylpyridazin-3-yl)amino]benzimidazol-1-yl]-2-pyridyl]pyrrolidine-3-carbonitrile

Error! Objects cannot be created from editing field codes.

Following the procedure described in step 2 of example 98, the title compound (125 mg, 47.3% yield) was obtained as a yellow solid. Purification by preparative HPLC (Waters Xbridge (150 mm×25 mm×5 μm). Flow rate: 25 mL/min. Gradient: 25% to 55% CH₃CN in (10 mM NH₄HCO₃ in H₂O) (8 min) then 100% CH₃CN (2 min)). LC-MS: m/z=441.1 [M+H]⁺, ESI pos. ¹H NMR (400 MHz, DMSO-d₆): δ=9.15 (s, 1H), 8.84 (s, 1H), 8.31-8.27 (m, 1H), 8.18-8.13 (m, 1H), 7.98 (dd, J=3.5, 8.1 Hz, 1H), 7.57 (br d, J=9.0 Hz, 1H), 7.35-7.28 (m, 2H), 7.08 (d, J=9.0 Hz, 1H), 5.05 (d, J=6.5 Hz, 1H), 3.98-3.88 (m, 1H), 3.88-3.79 (m, 1H), 3.79-3.71 (m, 1H), 3.70-3.61 (m, 1H), 3.61-3.47 (m, 2H), 2.48 (s, 3H), 2.41-2.31 (m, 1H), 2.30-2.19 (m, 1H), 1.39 (dd, J=4.6, 5.9 Hz, 3H).

### Example 106

### (3R)-1-[3-(1-Hydroxyethyl)-6-[6-[(6-methylpyridazin-3-yl)amino]benzimidazol-1-yl]-2-pyridyl]pyrrolidine-3-carbonitrile

Following the procedure described in

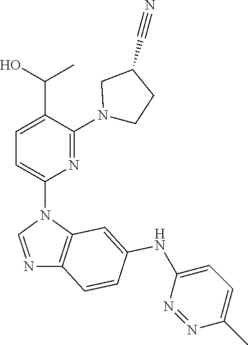 [SMILES: Cc1ccc(Nc2ccc3ncn(-c4ccc(C(C)O)c(N5CC[C@@H](C#N)C5)n4)c3c2)nn1]

step 2 of example 98, starting with (3R)-1-[3-acetyl-6-[6-[(6-methylpyridazin-3-yl)amino]benzimidazol-1-yl]-2-pyridyl]pyrrolidine-3-carbonitrile (obtained in step 2 of example 105-A) (250 mg, 0.6 mmol, 1 equiv.), the title compound (115 mg, 44.2% yield) was obtained as a yellow solid. Purification by preparative HPLC (Waters Xbridge (150 mm×25 mm×5 μm). Flow rate: 25 mL/min. Gradient: 23% to 53% CH₃CN in (10 mM NH₄HCO₃ in H2O) (10 min) then 100% CH₃CN (2 min)). LC-MS: m/z=441.1 [M+H]⁺, ESI pos. ¹H NMR (400 MHz, DMSO-d₆): δ=9.41 (dd, J=1.7, 6.2 Hz, 1H), 9.26 (s, 1H), 8.70 (d, J=4.8 Hz, 1H), 7.98 (dd, J=3.6, 8.1 Hz, 1H), 7.63 (d, J=8.6 Hz, 1H), 7.33 (d, J=9.2 Hz, 1H), 7.25 (dd, J=2.2, 7.9 Hz, 1H), 7.17 (br d, J=8.7 Hz, 1H), 7.09 (d, J=9.0 Hz, 1H), 5.07 (q, J=6.1 Hz, 1H), 4.19-4.09 (m, 1H), 3.91-3.81 (m, 1H), 3.77-3.70 (m, 2H), 3.57-3.44 (m, 2H), 2.49-2.48 (m, 3H), 2.40-2.31 (m, 1H), 2.30-2.19 (m, 1H), 1.41 (t, J=5.7 Hz, 3H).

### Example 107

### 1-[2-(3,5-Dimethylpyrazol-1-yl)-6-[6-[(6-methylpyridazin-3-yl)amino]benzimidazol-1-yl]-3-pyridyl]ethanol; formic acid

Error! Objects cannot be created from editing field codes.

### Step 1: 1-[6-chloro-2-(3,5-dimethylpyrazol-1-yl)-3-pyridyl]ethanone

Error! Objects cannot be created from editing field codes.

A solution of 3,5-dimethylpyrazole (200 mg, 2.08 mmol, 1 equiv.), 1-(6-chloro-2-fluoro-3-pyridyl)ethanone (CAS #1260663-13-5, 361 mg, 2.08 mmol, 1 equiv.) and DIPEA (810 mg, 6.27 mmol, 3.01 equiv.) in DMSO (3 mL) was stirred at 30° C. for 4 hours, then at 50° C. for another 4 hours, then at 80° C. for another 4 hours and then at 100° C. for another 14 hours. The reaction mixture was cooled to RT, quenched with H₂O (30 mL) and extracted with EtOAc (3×10 mL). The combined organic layers were dried over Na₂SO₄, filtered and concentrated under reduced pressure. The residue was purified by flash chromatography (silica gel, 0% to 20% EtOAc in petroleum ether. The title compound (290 mg, 44.7% yield) was obtained as a light brown solid. LC-MS: m/z=250.1 [M+H]⁺, ESI pos. ¹H NMR (400 MHz, CDCl₃): δ=7.86 (d, J=7.9 Hz, 1H), 7.31 (d, J=7.9 Hz, 1H), 6.07 (s, 1H), 2.58 (s, 3H), 2.24 (s, 3H), 2.06-2.05 (m, 3H).

### Step 2: 1-[2-(3,5-dimethylpyrazol-1-yl)-6-[5-[(6-methylpyridazin-3-yl)amino]benzimidazol-1-yl]-3-pyridyl]ethanone and 1-[2-(3,5-dimethylpyrazol-1-yl)-6-[6-[(6-methylpyridazin-3-yl)amino]benzimidazol-1-yl]-3-pyridyl]ethanone

Error! Objects cannot be created from editing field codes.

A solution of 1-[6-chloro-2-(3,5-dimethylpyrazol-1-yl)-3-pyridyl]ethanone (280 mg, 1.12 mmol, 1 equiv.), N-(6-methylpyridazin-3-yl)-1H-benzimidazol-5-amine (308 mg, 1.37 mmol, 1.22 equiv.) and K₂CO₃ (476 mg, 3.44 mmol, 3.07 equiv.) in DMSO (3 mL) was stirred at 50° C. for 16 hours, then at 80° C. for 4 hours. The reaction mixture was cooled to RT, quenched with H₂O (50 mL) and extracted with EtOAc (3×30 mL). The combined organic layers were dried over Na₂SO₄, filtered and concentrated under reduced pressure. The residue was purified by preparative NPLC (Welch Ultimate XB-CN (250 mm×50 mm×10 μm). Flow rate: 100 mL/min. Gradient: 30% to 70% EtOH in hexane (15 min) then 100% EtOH (5 min)) to give 1-[2-(3,5-dimethylpyrazol-1-yl)-6-[5-[(6-methylpyridazin-3-yl)amino]benzimidazol-1-yl]-3-pyridyl]ethanone (90 mg, 18.3% yield) as a brown solid and 1-[2-(3,5-dimethylpyrazol-1-yl)-6-[6-[(6-methylpyridazin-3-yl)amino]benzimidazol-1-yl]-3-pyridyl]ethanone (90 mg, 18.3% yield) as a brown solid. Regioisomer 1: LC-MS: m/z=439.0 [M+H]⁺, ESI pos. ¹H NMR (400 MHz, CDCl₃): δ=8.64 (s, 1H), 8.19 (d, J=8.3 Hz, 1H), 8.07 (br d, J=8.4 Hz, 1H), 7.80 (d, J=1.8 Hz, 1H), 7.57 (d, J=8.2 Hz, 1H), 7.40 (dd, J=1.7, 8.7 Hz, 1H), 7.24 (br d, J=5.0 Hz, 2H), 6.16 (s, 1H), 2.61 (d, J=7.6 Hz, 6H), 2.30 (s, 3H), 2.09 (s, 3H). Regioisomer 2: LC-MS: m/z=439.0 [M+H]⁺, ESI pos. ¹H NMR (400 MHz, CD₃OD): δ=8.85 (d, J=1.8 Hz, 1H), 8.81 (s, 1H), 8.34 (d, J=8.3 Hz, 1H), 7.96 (d, J=8.2 Hz, 1H), 7.69 (d, J=8.8 Hz, 1H), 7.47 (dd, J=2.0, 8.7 Hz, 1H), 7.35 (d, J=9.0 Hz, 1H), 7.13 (d, J=9.2 Hz, 1H), 6.20 (s, 1H), 2.55 (d, J=6.4 Hz, 6H), 2.25 (s, 3H), 2.04 (s, 3H).

### Step 3: 1-[2-(3,5-dimethylpyrazol-1-yl)-6-[6-[(6-methylpyridazin-3-yl)amino]benzimidazol-1-yl]-3-pyridyl]ethanol

Error! Objects cannot be created from editing field codes.

To a solution of 1-[2-(3,5-dimethylpyrazol-1-yl)-6-[6-[(6-methylpyridazin-3-yl)amino]benzimidazol-1-yl]-3-pyridyl]ethanone (80 mg, 0.18 mmol, 1 equiv.) in MeOH (1 mL) and DCM (9 mL) was added NaBH₄ (21 mg, 0.56 mmol, 3.04 equiv.) at 0° C. The reaction mixture was stirred at 0° C. for 15 minutes. The mixture was quenched with H₂O (30 mL) and extracted with DCM (3×15 mL). The combined organic layers were dried over Na₂SO₄, filtered and concentrated under reduced pressure. The residue was by preparative HPLC (Phenomenex Luna C18 (150 mm×25 mm×10 μm). Flow rate: 25 mL/min. Gradient: 7% to 37% CH₃CN in (0.225% formic acid in H₂O v/v) (10 min) then 100% CH₃CN (2 min)). The title compound (30.4 mg, 37.8% yield) was obtained as a light yellow liophilized solid. LC-MS: m/z=441.3 [M+H]⁺, ESI pos. ¹H NMR (400 MHz, CD₃OD): δ=8.76 (s, 1H), 8.72 (s, 1H), 8.49 (d, J=8.4 Hz, 1H), 8.05 (d, J=8.3 Hz, 1H), 7.69 (d, J=8.7 Hz, 1H), 7.46 (d, J=8.8 Hz, 1H), 7.34 (d, J=9.3 Hz, 1H), 7.15 (d, J=9.2 Hz, 1H), 6.13 (s, 1H), 4.84 (m, 1H), 2.55 (s, 3H), 2.30 (d, J=6.6 Hz, 6H), 1.36 (d, J =6.5 Hz, 3H).

### Example 108

### 1-[2-(3,5-Dimethylpyrazol-1-yl)-6-[5-[(6-methylpyridazin-3-yl)amino]benzimidazol-1-yl]-3-pyridyl]ethanol; formic acid

Error! Objects cannot be created from editing field codes.

Following the procedure described in step 3 of example 107, starting with 1-[2-(3,5-dimethylpyrazol-1-yl)-6-[5-[(6-methylpyridazin-3-yl)amino]benzimidazol-1-yl]-3-pyridyl]ethanone (80 mg, 0.18 mmol, 1 equiv.), the title compound (42.1 mg, 52.4% yield) was obtained as a light yellow solid. Purification by preparative HPLC (Phenomenex Luna C18 (150 mm×25 mm×10 μm). Flow rate: 25 mL/min. Gradient: 7% to 37% CH₃CN in (0.225% formic acid in H₂O v/v) (10 min) then 100% CH₃CN (2 min)). LC-MS: m/z=441.3 [M+H]⁺, ESI pos. ¹H NMR (400 MHz, CD₃OD): δ=8.88 (s, 1H), 8.43 (d, J=8.3 Hz, 1H), 8.21 (d, J=1.6 Hz, 1H), 8.15 (d, J=8.9 Hz, 1H), 8.04 (d, J=8.4 Hz, 1H), 7.61-7.55 (m, 1H), 7.38 (d, J=9.3 Hz, 1H), 7.15 (d, J=9.0 Hz, 1H), 6.18 (s, 1H), 4.84 (m, 1H), 2.54 (s, 3H), 2.32 (d, J=6.1 Hz, 6H), 1.36 (d, J=6.5 Hz, 3H).

### Example 109

### 5-Methyl-1-[6-[5-[(6-methylpyridazin-3-yl)amino]benzimidazol-1-yl]-3-[(1S)-1-hydroxyethyl]-2-pyridyl]pyrazole-3-carbonitrile

Error! Objects cannot be created from editing field codes.

1-[3-(1-hydroxyethyl)-6-[5-[(6-methylpyridazin-3-yl)amino]benzimidazol-1-yl]-2-pyridyl]-5-methyl-pyrazole-3-carbonitrile (obtained as in step 3 of example 64) (400 mg, 0.89 mmol, 1 equiv.) was purified by chiral SFC (Daicel Chiralpak AY-H (250 mm×30 mm×10 μm). Flow rate: 70 mL/min. 60% (0.1% NH₄OH in isopropanol). The title compound (157.5 mg, 39.4% yield) was obtained as a white solid. LC-MS: m/z=452.2 [M+H]⁺, ESI pos. ¹H NMR (400 MHz, CD₃OD): δ=8.89 (s, 1H), 8.50 (d, J=8.5 Hz, 1H), 8.22 (d, J=1.9 Hz, 1H), 8.13 (dd, J=8.7, 15.2 Hz, 2H), 7.60 (dd, J=2.1, 8.9 Hz, 1H), 7.35 (d, J=9.1 Hz, 1H), 7.12 (d, J=9.1 Hz, 1H), 6.87 (d, J=0.6 Hz, 1H), 4.78-4.74 (m, 1H), 2.53 (s, 3H), 2.41 (s, 3H), 1.41 (d, J=6.5 Hz, 3H).

Assignment of the absolute configuration of the stereogenic carbon was performed by Mosher ester analysis: 3.6 mg (8.0 μmol) of alcohol were dissolved in CDCl₃ and 2.9 μl of pyridine (36.3 μmol, 7 equiv.), were added followed by of (R)-Mosher chloride ((R)-α-Methoxy-α-trifluoromethylphenylacetyl chloride, 7.6 μl, 40.2 μmol, 5.0 equiv.) for the formation of the corresponding (S)-Mosher ester. Comparison with the ¹H-NMR spectrum (COSY) of example 110 by overlay shows an upfield shift of the C2-methyl group hence indicating an (S)-configuration of the alcohol.

### Example 110

### 5-Methyl-1-[6-[5-[(6-methylpyridazin-3-yl)amino]benzimidazol-1-yl]-3-[(1R)-1-hydroxyethyl]-2-pyridyl]pyrazole-3-carbonitrile

Error! Objects cannot be created from editing field codes.

1-[3-(1-hydroxyethyl)-6-[5-[(6-methylpyridazin-3-yl)amino]benzimidazol-1-yl]-2-pyridyl]-5-methyl-pyrazole-3-carbonitrile (obtained as in step 3 of example 64) (400 mg, 0.89 mmol, 1 equiv.) was purified by chiral SFC (Daicel Chiralpak AY-H (250 mm×30 mm×10 μm). Flow rate: 70 mL/min. 60% (0.1% NH₄OH in isopropanol)). The title compound (174.5 mg, 43.6% yield) was obtained as a white solid. LC-MS: m/z=452.2 [M+H]⁺, ESI pos. ¹H NMR (400 MHz, CD₃OD): δ=8.89 (s, 1H), 8.50 (d, J=8.4 Hz, 1H), 8.22 (d, J=2.0 Hz, 1H), 8.16-8.08 (m, 2H), 7.59 (dd, J=2.1, 8.9 Hz, 1H), 7.35 (d, J=9.3 Hz, 1H), 7.12 (d, J=9.1 Hz, 1H), 6.87 (s, 1H), 4.79-4.74 (m, 1H), 2.53 (s, 3H), 2.41 (s, 3H), 1.41 (d, J=6.5 Hz, 3H).

Assignment of the absolute configuration of the stereogenic carbon was performed by Mosher ester analysis: 1.5 mg (3.3 μmol) of alcohol were dissolved in CDCl₃ and 1.9 μl of pyridine (23.8 μmol, 7 equiv.), were added followed by of (R)-Mosher chloride ((R)-α-Methoxy-α-trifluoromethylphenylacetyl chloride, 3.1 μl, 16.5 μmol, 5.0 equiv.) for the formation of the corresponding (S)-Mosher ester.

Comparison with the ¹H-NMR spectrum (COSY) of example 109 by overlay shows a downfield shift of the C2-methyl group hence indicating an (R)-configuration of the alcohol.

### Example 111

### 1-[3-(1-Hydroxyethyl)-6-[5-[(5-methyl-1,3,4-thiadiazol-2-yl)amino]benzimidazol-1-yl]-2-pyridyl]-5-methyl-pyrazole-3-carbonitrile

Error! Objects cannot be created from editing field codes.

### Step 1: 1-[3-acetyl-6-[5-[(5-methyl-1,3,4-thiadiazol-2-yl)amino]benzimidazol-1-yl]-2-pyridyl]-5-methyl-pyrazole-3-carbonitrile

Error! Objects cannot be created from editing field codes.

According to the procedure described in step 3 of example 104, with (5-methyl-1,3,4-thiadiazol-2-yl)amine (27 mg, 0.237 mmol, 2 equiv.), the title compound (41.3 mg, 73.8% yield) was obtained as an off-white solid. LC-MS: m/z=456.3 [M+H]⁺, ESI pos.

### Step 2: 1-[3-(-hydroxyethyl)-6-[5-[(5-methyl-1,3,4-thiadiazol-2-yl)amino]benzimidazol-1-yl]-2-pyridyl]-5-methyl-pyrazole-3-carbonitrile

Error! Objects cannot be created from editing field codes.

Following the procedure described in step 2 of example 84, the title compound (9 mg, 21% yield) was obtained as a light yellow solid. LC-MS: m/z=458.3 [M+H]⁺, ESI pos.

### Example 113

### 1-[3-(1-Hydroxyethyl)-6-[5-[[1-(oxetan-3-yl)-4-piperidyl]oxy]benzimidazol-1-yl]-2-pyridyl]-5-methyl-pyrazole-3-carbonitrile

Error! Objects cannot be created from editing field codes.

### Step 1: 4-(3,4-dinitrophenoxy)-1-(oxetan-3-yl)piperidine

Error! Objects cannot be created from editing field codes.

1-(oxetan-3-yl)piperidin-4-ol (5.0 g, 31.8 mmol, 1 equiv.) and 3,4-dinitrofluorobenzene (6.51 g, 34.99 mmol, 1.1 equiv.) were dissolved in DMSO (8 mL), then potassium tert-butoxide (4.28 g, 38.17 mmol, 1.2 equiv.) was added and the mixture was stirred at 30° C. for 12 hours. The reaction mixture was then poured into H₂O (50 mL) and extracted with EtOAc (3×30 mL). The combined organic layers were dried over Na₂SO₄, filtered and concentrated under reduced pressure. The residue was first purified by flash chromatography (silica gel, 10% MeOH in DCM). The isolated material was then purified by preparative HPLC (Kromasil Eternity XT (250 mm×80 mm×10 μm). Flow rate: 100 mL/min. Gradient: 35% to 65% CH₃CN in (10 mM NH₄HCO₃ in H₂O) (18 min) then 100% CH₃CN (8 min)). The title compound (1.2 g, 11.7% yield) was obtained as a light brown solid. LC-MS: m/z=324.0 [M+H]⁺, ESI pos.

### Step 2: 4-[[1-(oxetan-3-yl)-4-piperidyl]oxy]benzene-1,2-diamine

Error! Objects cannot be created from editing field codes.

To a solution of 4-(3,4-dinitrophenoxy)-1-(oxetan-3-yl)piperidine (1.2 g, 3.71 mmol, 1 equiv.) in a mixture of MeOH (16 mL) and THF (8 mL) was added 10% Pd/C (197 mg, 0.190 mmol, 0.050 equiv.).

The mixture was stirred at 40° C. for 16 h under H₂ (50 psi). The mixture was filtered and the filtrate was concentrated under reduced pressure. The residue was purified by flash chromatography (silica gel, 0% to 20% MeOH in EtOAc). The title compound (800 mg, 81.8% yield)was obtained as a light brown solid. LC-MS: m/z=264.1 [M+H]⁺, ESI pos.

### Step 3: 5-[[1-(oxetan-3-yl)-4-piperidyl]oxy]-1H-benzimidazole

Error! Objects cannot be created from editing field codes.

A suspension of 4-[[1-(oxetan-3-yl)-4-piperidyl]oxy]benzene-1,2-diamine (800 mg, 3.04 mmol, 1 equiv.) in trimethyl orthoformate (6.45 g, 60.76 mmol, 20 equiv.) was stirred at 120° C. for 20 hours. The reaction mixture was cooled to RT and quenched with 1 N HCl (1.5 mL) and THF (1.5 mL). The mixture was then stirred at 30° C. for 12 hours. The pH of the reaction mixture was adjusted to 7 by the addition of 1 N HCl. The mixture was extracted with EtOAc (2×15 mL). The combined organic layers were concentrated under reduced pressure. The crude title (800 mg, 96.3% yield) was obtained as a light brown solid. LC-MS: m/z=274.1 [M+H]⁺, ESI pos.

### Step 4: 1-[3-acetyl-6-[5-[[1-(oxetan-3-yl)-4-piperidyl]oxy]benzimidazol-1-yl]-2-pyridyl]-5-methyl-pyrazole-3-carbonitrile and 1-[3-acetyl-6-[6-[[1-(oxetan-3-yl)-4-piperidyl]oxy]benzimidazol-1-yl]-2-pyridyl]-5-methyl-pyrazole-3-carbonitrile

Error! Objects cannot be created from editing field codes.

To a solution of 5-[[1-(oxetan-3-yl)-4-piperidyl]oxy]-1H-benzimidazole (400 mg, 1.46 mmol, 1 equiv.) in DMSO (8 mL) were added 1-(3-acetyl-6-chloro-2-pyridyl)-5-methyl-pyrazole-3-carbonitrile (obtained as in step 1 of example 64) (458 mg, 1.76 mmol, 1.2 equiv.) and K₂CO₃ (607 mg, 4.39 mmol, 3 equiv.). The reaction mixture was stirred at 30° C. for 12 hours. The mixture was then poured into H₂O (20 mL) and extracted with EtOAc (3×25 mL). The combined organic extracts were washed with brine (3×15 mL), dried over anhydrous Na₂SO₄, filtered and concentrated under reduced pressure. The residue was purified by flash chromatography (silica gel, 10% MeOH in DCM) to give 1-[3-acetyl-6-[5-[[1-(oxetan-3-yl)-4-piperidyl]oxy]benzimidazol-1-yl]-2-pyridyl]-5-methyl-pyrazole-3-carbonitrile (Rf 0.55, UV detection) (50 mg, 6.9% yield) as a light yellow oil and 1-[3-acetyl-6-[6-[[1-(oxetan-3-yl)-4-piperidyl]oxy]benzimidazol-1-yl]-2-pyridyl]-5-methyl-pyrazole-3-carbonitrile (Rf 0.6, UV detection) (50 mg, 6.9% yield) as a light yellow solid. Regioisomer 1: LC-MS: m/z=498.2 [M+H]⁺, ESI pos. ¹H NMR (400 MHz, CDCl₃): δ=8.60 (s, 1H), 8.30 (d, J=8.3 Hz, 1H), 7.93 (d, J=9.0 Hz, 1H), 7.75 (d, J=8.3 Hz, 1H), 7.39 (d, J=2.0 Hz, 1H), 7.28 (s, 1H), 7.07 (dd, J=1.9, 8.9 Hz, 1H), 6.73 (s, 1H), 4.70 (br d, J=6.5 Hz, 4H), 4.52-4.42 (m, 1H), 3.58 (br s, 1H), 2.70-2.60 (m, 5H), 2.32-2.21 (m, 5H), 2.12 (br d, J=3.4 Hz, 2H), 1.96 (br s, 2H). Regioisomer 2: LC-MS: m/z=498.2 [M+H]⁺, ESI pos. ¹H NMR (400 MHz, CDCl₃): δ=8.51 (s, 1H), 8.33-8.28 (m, 1H), 7.75 (d, J=8.6 Hz, 2H), 7.57 (d, J=2.1 Hz, 1H), 7.03 (dd, J=2.2, 8.8 Hz, 1H), 6.71 (s, 1H), 4.70-4.62 (m, 4H), 4.40-4.32 (m, 1H), 3.57-3.50 (m, 1H), 2.63-2.56 (m, 5H), 2.23-2.15 (m, 5H), 2.02 (br d, J=4.2 Hz, 2H), 1.94-1.84 (m, 2H).

### Step 5: 1-[3-(1-hydroxyethyl)-6-[5-[[1-(oxetan-3-yl)-4-piperidyl]oxy]benzimidazol-1-yl]-2-pyridyl]-5-methyl-pyrazole-3-carbonitrile

Error! Objects cannot be created from editing field codes.

To a clear light brown solution of 1-[3-acetyl-6-[5-[[1-(oxetan-3-yl)-4-piperidyl]oxy]benzimidazol-1-yl]-2-pyridyl]-5-methyl-pyrazole-3-carbonitrile (50 mg, 0.1 mmol, 1 equiv.) in a mixture of MeOH (2.5 mL) and DCM (0.5 mL) was added NaBH₄ (11 mg, 0.3 mmol, 3 equiv.) at 0° C. Stirring at 0° C. was continued for 1 hours. The mixture was quenched with saturated NH₄Cl (5 mL) at 0° C. and extracted with EtOAc (2×10 mL). The combined organic layers were dried over Na₂SO₄, filtered and concentracted under reduced pressure. The residue was purified by preparative HPLC: column Waters Xbridge (150 mm×25 mm×5 μm). Flow rate: 25 mL/min. Gradient: 25% to 55% CH₃CN in (10 mM NH₄HCO₃ in H₂O) (9 min) then 100% CH₃CN (0.5 min). The title compound (20.5 mg, 40.8% yield) was obtained as an off-white solid. LC-MS: m/z=500.2 [M+H]⁺, ESI pos. ¹H NMR (400 MHz, CD₃OD): δ=8.91 (s, 1H), 8.51 (d, J=8.4 Hz, 1H), 8.14 (d, J=8.4 Hz, 1H), 8.09 (d, J=9.0 Hz, 1H), 7.32 (d, J=2.1 Hz, 1H), 7.08 (dd, J=2.3, 9.0 Hz, 1H), 6.88 (s, 1H), 4.80-4.71 (m, 3H), 4.64 (d, J=6.4 Hz, 2H), 4.58-4.51 (m, 1H), 3.67-3.59 (m, 1H), 2.76-2.66 (m, 2H), 2.41 (s, 3H), 2.37 (br dd, J=4.3, 5.6 Hz, 2H), 2.13-2.07 (m, 2H), 1.94-1.86 (m, 2H), 1.43 (d, J=6.5 Hz, 3H).

### Example 114

### 1-[3-(1-Hydroxyethyl)-6-[6-[[1-(oxetan-3-yl)-4-piperidyl]oxy]benzimidazol-1-yl]-2-pyridyl]-5-methyl-pyrazole-3-carbonitrile

Error! Objects cannot be created from editing field codes.

Following the procedure described in step 5 of example 113, starting with 1-[3-acetyl-6-[6-[[1-(oxetan-3-yl)-4-piperidyl]oxy]benzimidazol-1-yl]-2-pyridyl]-5-methyl-pyrazole-3-carbonitrile (obtained in step 4 of example 113) (50 mg, 0.1 mmol, 1 equiv.) and using MeOH (2.5 mL) as solvent, the title compound (17 mg, 33.9% yield) was obtained as an off-white lyophilized solid. Purification by preparative HPLC (Waters Xbridge (150 mm×25 mm×5 μm). Flow rate: 25 mL/min. Gradient: 22% to 52% CH₃CN in (10 mM NH₄HCO₃ in H₂O) (9 min) then 100% CH₃CN (2 min)). LC-MS: m/z=508.2 [M+H]⁺, ESI pos. ¹H NMR (400 MHz, CD₃OD): δ=8.83 (s, 1H), 8.50 (d, J=8.4 Hz, 1H), 8.13 (d, J=8.6 Hz, 1H), 7.78 (d, J=2.1 Hz, 1H), 7.62 (d, J=8.8 Hz, 1H), 7.00 (dd, J=2.2, 8.8 Hz, 1H), 6.92 (s, 1H), 4.79-4.72 (m, 3H), 4.63 (t, J=6.2 Hz, 2H), 4.28 (br s, 1H), 3.58 (quin, J=6.4 Hz, 1H), 2.67 (br s, 2H), 2.39 (s, 3H), 2.12 (br s, 2H), 2.07-1.99 (m, 2H), 1.85-1.76 (m, 2H), 1.43 (d, J=6.5 Hz, 3H).

### Example 115

### 1-[3-(1-Hydroxyethyl)-6-[5-[(6-methylpyridazin-3-yl)amino]benzimidazol-1-yl]-2-pyridyl]pyrazole-3-carbonitrile

Error! Objects cannot be created from editing field codes.

### Step 1: 1-(3-acetyl-6-chloro-2-pyridyl)pyrazole-3-carbonitrile

Error! Objects cannot be created from editing field codes.

A solution of 1H-pyrazole-3-carbonitrile (200 mg, 2.15 mmol, 1 equiv.), 1-(6-chloro-2-fluoro-3-pyridyl)ethanone (CAS #1260663-13-5, 375 mg, 2.16 mmol, 1 equiv.) and DIPEA (840 mg, 6.5 mmol, 3 equiv.) in DMSO (4 mL) was stirred at 30° C. for 16 hours. The reaction mixture was quenched with H₂O (30 mL) and extracted with EtOAc (3×10 mL). The combined organic layers were dried over Na₂SO₄, filtered and concentrated under reduced pressure. The residue was purified by preparative TLC (silica gel, 20% EtOAc in petroleum ether, UV detection). The title compound (300 mg, 56.6% yield) was obtained as a white solid. LC-MS: m/z=247.2 [M+H]⁺, ESI pos. ¹H NMR (400 MHz, CDCl₃): δ=8.56 (d, J=2.7 Hz, 1H), 7.78 (d, J=7.9 Hz, 1H), 7.43 (d, J=7.9 Hz, 1H), 6.89 (d, J=2.7 Hz, 1H), 2.44 (s, 3H).

### Step 2: 1-[3-acetyl-6-[5-[(6-methylpyridazin-3-yl)amino]benzimidazol-1-yl]-2-pyridyl]pyrazole-3-carbonitrile

Error! Objects cannot be created from editing field codes.

Following the procedure described in step 4 of example 113, with N-(6-methylpyridazin-3-yl)-1H-benzimidazol-5-amine (obtained as in intermediate 1 of example 64) (319 mg, 1.42 mmol, 1.2 equiv.), the crude material was obtained and then first purified by preparative HPLC (Phenomenex Luna C18 (150 mm×25 mm×10 μm). Flow rate: 25 mL/min. Gradient: 11% to 41% CH₃CN in (0.225% formic acid in H₂O v/v) (10 min) then 100% CH₃CN (2 min)). Additional purification by preparative NPLC (Welch Ultimate XB-CN (250 mm×50 mm×10 μm). Flow rate: 100 mL/min. Gradient: 40% to 80% (EtOH with 0.1% NH₄OH v/v) in hexane (15 min) then 100% EtOH (5 min)) to give 1-[3-acetyl-6-[5-[(6-methylpyridazin-3-yl)amino]benzimidazol-1-yl]-2-pyridyl]pyrazole-3-carbonitrile (80 mg, 15.6% yield) as a brown solid and 1-[3-acetyl-6-[6-[(6-methylpyridazin-3-yl)amino]benzimidazol-1-yl]-2-pyridyl]pyrazole-3-carbonitrile (50 mg, 9.8% yield) as a brown solid. Regioisomer 1: LC-MS: m/z=436.3 [M+H]⁺, ESI pos. ¹H NMR (400 MHz, CD₃OD): δ=8.99 (s, 1H), 8.83 (d, J=2.7 Hz, 1H), 8.29-8.21 (m, 4H), 8.03 (d, J=8.3 Hz, 1H), 7.69 (dd, J=1.7, 8.8 Hz, 1H), 7.37 (d, J=9.3 Hz, 1H), 7.18-7.12 (m, 2H), 2.54 (s, 3H), 2.41 (s, 3H). Regioisomer 2: LC-MS: m/z=436.3 [M+H]⁺, ESI pos. ¹H NMR (400 MHz, CD₃OD): δ=9.44 (d, J=2.1 Hz, 1H), 9.18 (d, J=2.7 Hz, 1H), 8.92 (s, 1H), 8.26-8.23 (m, 2H), 8.04 (d, J=8.2 Hz, 1H), 7.68 (d, J=8.7 Hz, 1H), 7.39 (d, J=9.2 Hz, 1H), 7.30 (dd, J=2.0, 8.7 Hz, 1H), 7.15 (d, J=9.3 Hz, 1H), 7.08 (d, J=2.8 Hz, 1H), 2.59 (s, 3H), 2.42 (s, 3H).

### Step 3: 1-[3-(-hydroxyethyl)-6-[5-[(6-methylpyridazin-3-yl)amino]benzimidazol-1-yl]-2-pyridyl]pyrazole-3-carbonitrile

Error! Objects cannot be created from editing field codes.

Following the procedure described in step 3 of example 107, the title compound (6.9 mg, 8.2% yield) was obtained as a light yellow liophilized solid. Purification by preparative HPLC (Phenomenex Luna C18 (150 mm×25 mm×10 μm). Flow rate: 25 mL/min. Gradient: 9% to 39% CH₃CN in (0.225% formic acid in H₂O v/v) (10 min) then 100% CH₃CN (2 min)). LC-MS: m/z=438.1 [M+H]⁺, ESI pos. ¹H NMR (400 MHz, CD₃OD): δ=8.93 (s, 1H), 8.68 (d, J=2.7 Hz, 1H), 8.54 (d, J=8.2 Hz, 1H), 8.23 (d, J=1.8 Hz, 1H), 8.17 (d, J=8.9 Hz, 1H), 8.04 (d, J=8.6 Hz, 1H), 7.64 (dd, J=1.9, 8.9 Hz, 1H), 7.37 (d, J=9.3 Hz, 1H), 7.16-7.11 (m, 2H), 5.50 (q, J=6.2 Hz, 1H), 2.54 (s, 3H), 1.51 (d, J=6.4 Hz, 3H).

### Example 116

### 1-[[[3-(1-Hydroxyethyl)-6-[5-[(6-methylpyridazin-3-yl)amino]benzimidazol-1-yl]-2-pyridyl]amino]methyl]cyclopropanecarbonitrile; formic acid

Error! Objects cannot be created from editing field codes.

### Step 1: 1-[[(3-acetyl-6-chloro-2-pyridyl)amino]methyl]cyclopropanecarbonitrile

Error! Objects cannot be created from editing field codes.

To a solution of 1-(6-chloro-2-fluoro-3-pyridyl)ethanone (CAS #1260663-13-5, 1.05 g, 6.05 mmol, 1 equiv.), 1-(aminomethyl)cyclopropanecarbonitrile hydrochloride (963 mg, 7.26 mmol, 1.2 equiv.) in DMSO (20 mL) was added DIPEA (3 mL, 18.15 mmol, 3 equiv.) stirring was continued at 30° C. for 12 hours. The mixture was poured into H₂O (200 mL) and extracted with EtOAc (3×100 mL). The combined organic layers were as washed with brine (3×100 mL), dried over Na₂SO₄, filtered and concentrated in vacuo. The crude title compound (1.5 g, 99.3% yield) was obtained as a yellow solid. LC-MS: m/z=250.0 [M+H]⁺, ESI pos.

### Step 2: 1-[[[3-acetyl-6-56-[(6-methylpyridazin-3-yl)amino]benzimidazol-1-yl]-2-pyridyl]amino]methyl]cyclopropanecarbonitrile and 1-[[[3-acetyl-6-[6-[(6-methylpyridazin-3-yl)amino]benzimidazol-1-yl]-2-pyridyl]amino]methyl]cyclopropanecarbonitrile

Error! Objects cannot be created from editing field codes.

Following the procedure described in step 2 of example 115, the crude material was obtained and purified by preparative NPLC (Welch Ultimate XB-SiOH (250 mm×70 mm×10 μm). Flow rate: 140 mL/min. Gradient: 20% to 60% (EtOH) in heptane (20 min) then 100% EtOH (3 min)) to give 1-[[[3-acetyl-6-[5-[(6-methylpyridazin-3-yl)amino]benzimidazol-1-yl]-2-pyridyl]amino]methyl]cyclopropanecarbonitrile (200 mg, 28.5% yield) as a brown solid and 1-[[[3-acetyl-6-[6-[(6-methylpyridazin-3-yl)amino]benzimidazol-1-yl]-2-pyridyl]amino]methyl]cyclopropanecarbonitrile (170 mg, 24.2% yield) as a yellow solid. Regioisomer 1: LC-MS: m/z=439.1 [M+H]⁺, ESI pos. ¹H NMR (400 MHz, DMSO-d₆): δ=9.56 (t, J=5.6 Hz, 1H), 9.40 (d, J=1.9 Hz, 1H), 9.32 (s, 1H), 8.93 (s, 1H), 8.47 (d, J=8.5 Hz, 1H), 7.67 (d, J=8.8 Hz, 1H), 7.36 (d, J=9.1 Hz, 1H), 7.24 (dd, J=2.0, 8.6 Hz, 1H), 7.19 (d, J=8.4 Hz, 1H), 7.12 (d, J=9.1 Hz, 1H), 4.03-3.94 (m, 2H), 2.62 (s, 3H), 2.49-2.48 (m, 3H), 1.16-1.12 (m, 2H), 1.03-0.98 (m, 2H). Regioisomer 2: LC-MS: m/z=439.1 [M+H]⁺, ESI pos. ¹H NMR (400 MHz, DMSO-d₆): δ=9.50 (br t, J =6.1 Hz, 1H), 9.35 (s, 1H), 9.06 (s, 1H), 8.45 (d, J=8.5 Hz, 1H), 8.38 (d, J=2.0 Hz, 1H), 8.26 (d, J=8.9 Hz, 1H), 7.62 (dd, J=2.0, 8.9 Hz, 1H), 7.37 (d, J=9.0 Hz, 1H), 7.23 (d, J=8.5 Hz, 1H), 7.15 (d, J=9.1 Hz, 1H), 3.88 (d, J=6.0 Hz, 2H), 2.61 (s, 3H), 2.49-2.49 (m, 3H), 1.26 (t, J=3.0 Hz, 2H), 1.21-1.16 (m, 2H), 1.05 (t, J=7.0 Hz, 1H).

### Step 3: 1-[[[3-(1-hydroxyethyl)-6-[5-[(6-methylpyridazin-3-yl)amino]benzimidazol-1-yl]-2-pyridyl]amino]methyl]cyclopropanecarbonitrile; formic acid

Error! Objects cannot be created from editing field codes.

Following the procedure described in step 3 of example 107, using a mixture of MeOH (5 mL) and DMF (3 mL) as solvent, the title compound (39.5 mg, 21.8% yield) was obtained as a white liophilized solid.

Purification by preparative HPLC (Phenomenex Luna C18 (150 mm×25 mm×10 μm). Flow rate: 25 mL/min. Gradient: 6% to 36% CH₃CN in (0.225% formic acid in H₂O v/v) (10 min) then 100% CH₃CN (2 min)). LC-MS: m/z=441.3 [M+H]⁺, ESI pos. ¹H NMR (400 MHz, DMSO-d₆): δ=9.18 (br s, 1H), 8.82 (s, 1H), 8.36 (d, J=1.8 Hz, 1H), 8.15 (s, 1H), 8.09 (d, J=8.8 Hz, 1H), 7.65 (d, J=7.8 Hz, 1H), 7.52 (dd, J=1.8, 8.9 Hz, 1H), 7.32 (d, J=9.0 Hz, 1H), 7.08 (d, J=9.0 Hz, 1H), 7.03 (d, J=7.8 Hz, 1H), 6.84 (t, J=6.0 Hz, 1H), 4.86 (q, J=6.2 Hz, 1H), 3.74 (br dd, J=2.4, 5.7 Hz, 2H), 2.47 (s, 3H), 1.40 (d, J=6.4 Hz, 3H), 1.22-1.17 (m, 2H), 1.16-1.10 (m, 2H).

### Example 117

### 1-[[[3-(1-Hydroxyethyl)-6-[6-[(6-methylpyridazin-3-yl)amino]benzimidazol-1-yl]-2-pyridyl]amino]methyl]cyclopropanecarbonitrile; formic acid

Error! Objects cannot be created from editing field codes.

Following the procedure described in step 3 of example 115, with 1-[[[3-acetyl-6-[6-[(6-methylpyridazin-3-yl)amino]benzimidazol-1-yl]-2-pyridyl]amino]methyl]cyclopropanecarbonitrile (obtained in step 2 of example 116) (180 mg, 0.41 mmol, 1 equiv.), the title compound (69 mg, 37% yield) was obtained as a white liophilized solid. Purification by preparative HPLC (Phenomenex Luna C18 (150 mm×25 mm×10 μm). Flow rate: 25 mL/min. Gradient: 6% to 36% CH₃CN in (0.225% formic acid in H₂O v/v) (10 min) then 100% CH₃CN (2 min)). LC-MS: m/z=441.2 [M+H]⁺, ESI pos. ¹H NMR (400 MHz, DMSO-d₆): δ=9.26 (br s, 1H), 9.18 (d, J=1.8 Hz, 1H), 8.69 (s, 1H), 8.19 (s, 1H), 7.65 (dd, J=3.4, 8.3 Hz, 2H), 7.33 (d, J=9.0 Hz, 1H), 7.23 (dd, J=2.0, 8.7 Hz, 1H), 7.10 (d, J=9.0 Hz, 1H), 6.98 (d, J=7.8 Hz, 1H), 6.88 (t, J=5.7 Hz, 1H), 4.90 (q, J=6.4 Hz, 1H), 3.86-3.74 (m, 2H), 2.48 (s, 3H), 1.43 (d, J=6.4 Hz, 3H), 1.11-1.06 (m, 2H), 1.05-0.99 (m, 2H).

### Example 118

### 5-[3-(1-Hydroxyethyl)-6-[5-[(6-methylpyridazin-3-yl)amino]benzimidazol-1-yl]-2-pyridyl]pyridine-3-carbonitrile; formic acid

Error! Objects cannot be created from editing field codes.

### Step 1: 5-[3-acetyl-6-[5-[(6-methylpyridazin-3-yl)amino]benzimidazol-1-yl]-2-pyridyl]pyridine-3-carbonitrile

Error! Objects cannot be created from editing field codes.

Following the procedure described in step 3 of example 76, with 5-cyanopyridine-3-boronic acid (52 mg, 0.35 mmol, 1.2 equiv.), with a reaction time of 3 h, the obtained crude material was purified preparative TLC (silica gel, 10% MeOH in DCM, UV detection). The title compound (110 mg, 84.8% yield) was obtained as a yellow solid. LC-MS: m/z=447.3 [M+H]⁺, ESI pos.

### Step 2: 5-[3-(1-hydroxyethyl)-6-[5-[(6-methylpyridazin-3-yl)amino]benzimidazol-1-yl]-2-pyridyl]pyridine-3-carbonitrile

Error! Objects cannot be created from editing field codes.

Following the procedure described in step 5 of example 113, the title compound (20 mg, 16.3% yield) was obtained as a brown lyophilized solid. Purification by preparative HPLC (Phenomenex Luna C18 (150 mm×25 mm×10 μm). Flow rate: 25 mL/min. Gradient: 5% to 35% CH₃CN in (0.225% formic acid in H₂O v/v) (10 min) then 100% CH₃CN (2 min)). LC-MS: m/z=449.1 [M+H]⁺, ESI pos. ¹H NMR (400 MHz, DMSO-d₆): δ=9.21 (s, 1H), 9.17 (br s, 2H), 9.01 (s, 1H), 8.67 (s, 1H), 8.42 (s, 1H), 8.32 (br d, J=8.6 Hz, 1H), 8.11 (br dd, J=8.7, 15.5 Hz, 2H), 7.48 (br d, J=8.4 Hz, 1H), 7.32 (br d, J=8.9 Hz, 1H), 7.08 (br d, J=9.0 Hz, 1H), 4.85-4.76 (m, 1H), 3.17 (s, 1H), 2.47 (br s, 3H), 1.39 (br d, J=6.2 Hz, 3H).

### Example 119

### 1-[3-(1-Hydroxyethyl)-6-[5-(1,2,4-triazin-3-ylamino)benzimidazol-1-yl]-2-pyridyl]-5-methyl-pyrazole-3-carbonitrile

Error! Objects cannot be created from editing field codes.

### Step 1: 1-[3-acetyl-6-[5-(1,2,4-triazin-3-ylamino)benzimidazol-1-yl]-2-pyridyl]-5-methyl-pyrazole-3-carbonitrile

Error! Objects cannot be created from editing field codes.

Following the procedure described in step 3 of example 104, with 1,2,4-triazin-3-ylamine (23 mg, 0.237 mmol, 2 equiv.), the title compound (56.7 mg, quantitative yield) was obtained as a yellow solid. LC-MS: m/z=437.3 [M+H]⁺, ESI pos.

### Step 2: 1-[3-(1-hydroxyethyl)-6-[5-(1,2,4-triazin-3-ylamino)benzimidazol-1-yl]-2-pyridyl]-5-methyl-pyrazole-3-carbonitrile

Error! Objects cannot be created from editing field codes.

Following the procedure described in step 2 of example 84, the title compound (8.4 mg, 14.5% yield) was obtained as a yellow solid. LC-MS: m/z=439.2 [M+H]⁺, ESI pos.

### Example 120

### 1-[3-(1-Hydroxyethyl)-6-[6-[(6-methylpyridazin-3-yl)amino]benzimidazol-1-yl]-2-pyridyl]-3-methyl-pyrrolidine-3-carbonitrile

Error! Objects cannot be created from editing field codes.

### Step 1: 1-(3-acetyl-6-chloro-2-pyridyl)-3-methyl-pyrrolidine-3-carbonitrile

Error! Objects cannot be created from editing field codes.

A solution of 1-(6-chloro-2-fluoro-3-pyridyl)ethanone (CAS #1260663-13-5, 400 mg, 2.3 mmol, 1 equiv.), 3-methylpyrrolidine-3-carbonitrile hydrochloride (338 mg, 2.3 mmol, 1 equiv.) and DIPEA (1.14 mL, 6.91 mmol, 3 equiv.) in DMSO (4 mL) was stirred at 100° C. for 3 hours. The reaction mixture was cooled to RT, poured into H₂O (10 mL9 and extracted with EtOAc (2×10 mL). The combined organic layers were washed with brine (2×10 mL) and concentrated to dryness. The crude title compound (300 mg, 98.7% yield) was obtained as a yellow oil. LC-MS: m/z=264.2 [M+H]⁺, ESI pos.

### Step 2: 1-[3-acetyl-6-[5-[(6-methylpyridazin-3-yl)amino]benzimidazol-1-yl]-2-pyridyl]-3-methyl-pyrrolidine-3-carbonitrile; formic acid and 1-[3-acetyl-6-[6-[(6-methylpyridazin-3-yl)amino]benzimidazol-1-yl]-2-pyridyl]-3-methyl-pyrrolidine-3-carbonitrile; formic acid

Error! Objects cannot be created from editing field codes.

Following the procedure described in step 4 of example 113, with a reaction time of 16 h at 100° C., the reaction mixture was cooled to RT, filtered and directly purified by preparative HPLC (Phenomenex Luna C18 (150 mm×40 mm×15 μm). Flow rate: 60 mL/min. Gradient: 10% to 40% CH₃CN in (0.225% formic acid in H₂O v/v) (13 min) then 100% CH₃CN (2 min)). A mixture (454 mg) of both title compounds was obtained. This mixture was further purified by preparative NPLC (Welch Ultimate XB-SiOH (250 mm×70 mm×10 μm). Flow rate: 140 mL/min. Gradient: 45% to 85% EtOH in hexane (12 min) then 100% EtOH (5 min)) to give 1-[3-acetyl-6-[5-[(6-methylpyridazin-3-yl)amino]benzimidazol-1-yl]-2-pyridyl]-3-methyl-pyrrolidine-3-carbonitrile; formic acid (200 mg, 29.1% yield) as a yellow solid and 1-[3-acetyl-6-[5-[(6-methylpyridazin-3-yl)amino]benzimidazol-1-yl]-2-pyridyl]-3-methyl-pyrrolidine-3-carbonitrile; formic acid (190 mg, 27.7% yield) as a light yellow oil. Regioisomer 1: LC-MS: m/z=453.3 [M+H]⁺, ESI pos. ¹H NMR (400 MHz, CDCl₃): δ=8.61-8.57 (m, 1H), 8.16-8.08 (m, 2H), 7.76-7.72 (m, 1H), 7.43-7.39 (m, 1H), 7.14 (s, 1H), 6.97-6.93 (m, 1H), 5.31 (s, 1H), 4.00-3.88 (m, 1H), 3.84-3.75 (m, 1H), 3.50-3.41 (m, 2H), 2.67-2.64 (m, 3H), 2.62-2.59 (m, 3H), 2.57-2.49 (m, 1H), 2.17-2.11 (m, 1H), 1.63-1.59 (m, 3H). Regioisomer 2: LC-MS: m/z=453.3 [M+H]⁺, ESI pos.

### Step 3: 1-[3-(1-hydroxyethyl)-6-[6-[(6-methylpyridazin-3-yl)amino]benzimidazol-1-yl]-2-pyridyl]-3-methyl-pyrrolidine-3-carbonitrile

Error! Objects cannot be created from editing field codes.

Following the procedure described in step 5 of example 113, the title compound (88.9 mg, 22.1% yield) was obtained as a white lyophilized solid. Purification by preparative HPLC (Waters Xbridge (150 mm×25 mm×5 μm). Flow rate: 25 mL/min. Gradient: 27% to 57% CH₃CN in (10 mM NH₄HCO₃ in H₂O) (8 min) then 100% CH₃CN (2 min)). LC-MS: m/z=455.2 [M+H]⁺, ESI pos. ¹H NMR (400 MHz, CDCl₃): δ=8.54-8.47 (m, 1H), 8.42-8.35 (m, 1H), 7.96-7.89 (m, 1H), 7.75-7.68 (m, 1H), 7.66-7.38 (m, 1H), 7.22-7.17 (m, 1H), 7.16-7.12 (m, 1H), 7.10-7.05 (m, 1H), 6.98-6.92 (m, 1H), 5.23-5.14 (m, 1H), 4.13-3.86 (m, 1H), 3.78-3.56 (m, 2H), 2.64-2.57 (m, 3H), 2.49-2.41 (m, 1H), 2.05-1.99 (m, 1H), 1.61-1.53 (m, 3H), 1.52-1.47 (m, 3H).

### Example 121

### 1-[3-(1-Hydroxyethyl)-6-[5-[(6-methylpyridazin-3-yl)amino]benzimidazol-1-yl]-2-pyridyl]-3-methyl-pyrrolidine-3-carbonitrile

Error! Objects cannot be created from editing field codes.

Following the procedure described in step 5 of example 113, with 1-[3-acetyl-6-[5-[(6-methylpyridazin-3-yl)amino]benzimidazol-1-yl]-2-pyridyl]-3-methyl-pyrrolidine-3-carbonitrile (obtained in step 2 of example 120) (150 mg, 0.33 mmol, 1 equiv.), the title compound (52.5 mg, 34.3% yield) was obtained as a white liophilized solid. Purification by preparative HPLC (Waters Xbridge (150 mm×25 mm×5 μm). Flow rate: 25 mL/min. Gradient: 27% to 57% CH₃CN in (10 mM NH₄HCO₃ in H₂O) (8 min) then 100% CH₃CN (2 min)). LC-MS: m/z=455.3 [M+H]⁺, ESI pos. ¹H NMR (400 MHz, DMSO-d₆): δ=9.20-9.12 (m, 1H), 8.88-8.80 (m, 1H), 8.33-8.28 (m, 1H), 8.17-8.10 (m, 1H), 8.03-7.93 (m, 1H), 7.62-7.54 (m, 1H), 7.35-7.27 (m, 2H), 7.12-7.04 (m, 1H), 5.34-5.21 (m, 1H), 5.09-5.00 (m, 1H), 4.08-3.97 (m, 1H), 3.91-3.62 (m, 3H), 2.48 (s, 3H), 2.45-2.39 (m, 1H), 2.18-2.05 (m, 1H), 1.59-1.49 (m, 3H), 1.47-1.33 (m, 3H).

### Example 122

### 1-[6-[5-[(6-Methylpyridazin-3-yl)amino]benzimidazol-1-yl]-2-[2-methyl-5-(trifluoromethyl)pyrazol-3-yl]-3-pyridyl]ethanol

Error! Objects cannot be created from editing field codes.

### Step 1: 1-[6-[5-[(6-methylpyridazin-3-yl)amino]benzimidazol-1-yl]-2-[2-methyl-5-(trifluoromethyl)pyrazol-3-yl]-3-pyridyl]ethanone

Error! Objects cannot be created from editing field codes.

Following the procedure described in step 3 of example 76, with 1-methyl-3-(trifluoromethyl)pyrazole-5-boronic acid pinacol ester (41 mg, 0.150 mmol, 1.4 equiv.), the reaction mixture was cooled to RT and filtered. The filter cake was taken in MeOH (30 mL) and the suspension was stirred at 30° C. for 1 hour.

The mixture was filtered and the filtrate was concentrated to leave the title compound (35 mg, 67.3% yield) as a yellow solid. LC-MS: m/z=493.2 [M+H]⁺, ESI pos.

### Step 2: 1-[6-[5-[(6-methylpyridazin-3-yl)amino]benzimidazol-1-yl]-2-[2-methyl-5-(trifluoromethyl)pyrazol-3-yl]-3-pyridyl]ethanol

Error! Objects cannot be created from editing field codes.

Following the procedure described in step 5 of example 113, the title compound (2.4 mg, 6.4% yield) was obtained as a white liophilized solid. LC-MS: m/z=495.2 [M+H]⁺, ESI pos. ¹H NMR (400 MHz, CD₃OD): δ=8.90 (s, 1H), 8.39 (d, J=8.8 Hz, 1H), 8.24 (d, J=2.0 Hz, 1H), 8.14 (d, J=8.8 Hz, 1H), 8.05 (d, J=8.8 Hz, 1H), 7.64-7.55 (m, 1H), 7.35 (d, J=9.2 Hz, 1H), 7.13 (d, J=9.2 Hz, 1H), 6.92 (s, 1H), 4.98-4.95 (m, 1H), 3.95 (s, 3H), 2.53 (s, 3H), 1.47 (d, J=6.4 Hz, 3H).

### Example 123

### 1-[3-(1-Hydroxyethyl)-6-[5-(isoxazol-3-ylamino)benzimidazol-1-yl]-2-pyridyl]-5-methyl-pyrazole-3-carbonitrile

Error! Objects cannot be created from editing field codes.

### Step 1: 1-[3-acetyl-6-[5-(isoxazol-3-ylamino)benzimidazol-1-yl]-2-pyridyl]-5-methyl-pyrazole-3-carbonitrile

Error! Objects cannot be created from editing field codes.

Following the procedure described in step 3 of example 104, with 3-aminoisoxazole (21 mg, 19 μL, 0.237 mmol, 2 equiv.), the title compound (9.3 mg, 17.2% yield) was obtained as a light yellow solid. LC-MS: m/z=425.3 [M+H]⁺, ESI pos.

### Step 2: 1-[3-(1-hydroxyethyl)-6-[5-(isoxazol-3-ylamino)benzimidazol-1-yl]-2-pyridyl]-5-methyl-pyrazole-3-carbonitrile

Error! Objects cannot be created from editing field codes.

Following the procedure described in step 2 of example 84, the title compound (6.1 mg, 65.7% yield) was obtained as a light yellow solid. LC-MS: m/z=427.2 [M+H]⁺, ESI pos.

### Example 124

### 3-(1-Hydroxyethyl)-6-[5-[(6-methylpyridazin-3-yl)amino]benzimidazol-1-yl]-N-(2,2,2-trifluoroethyl)pyridine-2-carboxamide; formic acid

Error! Objects cannot be created from editing field codes.

### Step 1: 3-acetyl-6-[5-[(6-methylpyridazin-3-yl)amino]benzimidazol-1-yl]-N-(2,2,2-trifluoroethyl)pyridine-2-carboxamide

Error! Objects cannot be created from editing field codes.

To a suspension of 1-[2-chloro-6-[5-[(6-methylpyridazin-3-yl)amino]benzimidazol-1-yl]-3-pyridyl]ethanone (obtained as in step 2 of example 76) (200 mg, 0.53 mmol, 1 equiv.) in 2,2,2-trifluoroethylamine (3.0 mL, 142.43 mmol, 270 equiv.) and DMF (3 mL) were added dppf (117 mg, 0.21 mmol, 0.4 equiv.), Pd(OAc)₂ (24 mg, 0.11 mmol, 0.2 equiv.) and NEt₃ (0.22 mL, 1.58 mmol, 3 equiv.).

The reaction mixture was then stirred under CO (50 psi) at 70° C. for 16 hours. The reaction mixture was cooled to RT, poured into H₂O (50 mL) and extracted with DCM (4×50 mL). The combined organics were washed with brine, dried over Na₂SO₄, filtered and concentrated in vacuo. The residue was purified by preparative TLC (silica gel, 10% MeOH in DCM, UV detection). The title compound (50 mg, 20.2% yield) was obtained as light brown solid. LC-MS: m/z=470.3 [M+H]⁺, ESI pos.

### Step 2: 3-(1-hydroxyethyl)-6-[5-[(6-methylpyridazin-3-yl)amino]benzimidazol-1-yl]-N-(2,2,2-trifluoroethyl)pyridine-2-carboxamide; formic acid

Error! Objects cannot be created from editing field codes.

To a solution of 3-acetyl-6-[5-[(6-methylpyridazin-3-yl)amino]benzimidazol-1-yl]-N-(2,2,2-trifluoroethyl)pyridine-2-carboxamide (58 mg, 0.12 mmol, 1 equiv.) in a mixture of MeOH (2 mL) and DMF (2 mL) was added NaBH₄ (14 mg, 0.37 mmol, 3 equiv.) at 0° C. The mixture was then stirred at 20° C. for 1 hour. The reaction mixture was directly purified by preparative HPLC: column Phenomenex Luna C18 (150 mm×25 mm×10 μm). Flow rate: 25 mL/min. Gradient: 10% to 40% CH₃CN in (0.225% formic acid in H₂O v/v) (10 min) then 100% CH₃CN (2 min). The title compound (9.1 mg, 14.8% yield) was obtained as yellow liophilized solid. LC-MS: m/z=472.1 [M+H]⁺, ESI pos. ¹H NMR (400 MHz, CD₃OD): δ=9.05 (s, 1H), 8.49 (d, J=8.6 Hz, 1H), 8.24 (d, J=2.0 Hz, 1H), 8.22 (d, J=8.9 Hz, 1H), 8.11 (s, 1H), 8.09 (s, 1H), 7.62 (dd, J=2.1, 8.8 Hz, 1H), 7.59 (d, J=9.2 Hz, 1H), 7.38 (d, J=9.3 Hz, 1H), 5.75 (q, J=6.3 Hz, 1H), 4.25-4.11 (m, 2H), 2.61 (s, 3H), 1.54 (d, J=6.4 Hz, 3H).

### Example 125

### 1-[6-[6-[(6-Methylpyridazin-3-yl)amino]benzimidazol-1-yl]-2-[(3aR,6aS)-2,3,3a,5,6,6a-hexahydro-1H-pyrrolo[3,2-b]pyrrol-4-yl]-3-pyridyl]ethanol; formic acid

Error! Objects cannot be created from editing field codes.

### Step 1: rac-trans-i-(3-acetyl-6-chloro-2-pyridyl)-2,3,3a,5,6,6a-hexahydropyrrolo[3,2-b]pyrrole-4-carboxylate

Error! Objects cannot be created from editing field codes.

Following the procedure described in step 4 of example 113, with rac-trans-hexahydro-pyrrolo[3,2-b]pyrrole-1-carboxylic acid tert-butyl ester (856 mg, 4.03 mmol, 1 equiv.), the title compound (1.28 g, 86.1% yield) was obtained as a colorless oil. LC-MS: m/z=366.1 [M+H]⁺, ESI pos.

### Step 2: tert-butyl rac-trans-i-[3-acetyl-6-[5-[(6-methylpyridazin-3-yl)amino]benzimidazol-1-yl]-2-pyridyl]-2,3,3a,5,6,6a-hexahydropyrrolo[3,2-b]pyrrole-4-carboxylate and tert-butyl rac-trans-1-[3-acetyl-6-[6-[(6-methylpyridazin-3-yl)amino]benzimidazol-1-yl]-2-pyridyl]-2,3,3a,5,6,6a-hexahydropyrrolo[3,2-b]pyrrole-4-carboxylate

Error! Objects cannot be created from editing field codes.

Following the procedure described in step 2 of example 120, with additional reaction time of 6 h at 110° C., the reaction mixture was cooled to RT, poured into H₂O (100 mL) and extracted with DCM (3×100 mL). The combined organic extracts were washed with brine, dried over Na₂SO₄, filtered and concentrated under vacuum. The residue was purified by preparative HPLC (Phenomenex Luna C18 (250 mm×70 mm×10 μm). Flow rate: 140 mL/min. Gradient: 23% to 50% CH₃CN in (0.225% formic acid in H₂O v/v) (20 min) then 100% CH₃CN (5 min)). A mixture of both title compounds (800 mg) was isolated and further purified by preparative NPLC (Welch Ultimate XB-CN (250 mm×70 mm×10 μm).

Flow rate: 140 mL/min. Gradient: 30% to 70% (EtOH with 0.1% NH₄OH v/v) in hexane (12 min) then 100% EtOH (5 min)). To give tert-butyl rac-trans-1-[3-acetyl-6-[5-[(6-methylpyridazin-3-yl)amino]benzimidazol-1-yl]-2-pyridyl]-2,3,3a,5,6,6a-hexahydropyrrolo[3,2-b]pyrrole-4-carboxylate (350 mg, 18% yield) as a yellow solid and tert-butyl rac-trans-1-[3-acetyl-6-[6-[(6-methylpyridazin-3-yl)amino]benzimidazol-1-yl]-2-pyridyl]-2,3,3a,5,6,6a-hexahydropyrrolo[3,2-b]pyrrole-4-carboxylate (420 mg, 21.6% yield) as a yellow solid. Regioisomer 1: LC-MS: m/z=555.3 [M+H]⁺, ESI pos. ¹H NMR (400 MHz, CDCl₃): δ=8.47 (s, 1H), 8.02 (d, J=8.1 Hz, 1H), 7.92 (d, J=8.8 Hz, 1H), 7.70 (d, J=2.0 Hz, 1H), 7.34-7.28 (m, 1H), 7.12-7.05 (m, 2H), 6.91 (d, J=8.1 Hz, 1H), 4.12 (dt, J=6.8, 10.8 Hz, 1H), 3.82-3.71 (m, 3H), 3.06 (br dd, J=8.5, 10.8 Hz, 1H), 2.55 (s, 3H), 2.52 (s, 3H), 2.51-2.40 (m, 3H), 2.03-1.89 (m, 1H), 1.79 (quin, J=10.5 Hz, 1H), 1.42 (s, 9H). Regioisomer 2: LC-MS: m/z=555.3 [M+H]⁺, ESI pos. ¹H NMR (400 MHz, CDCl₃): δ=8.74-8.59 (m, 1H), 8.36 (s, 1H), 8.00 (d, J=8.1 Hz, 1H), 7.69 (d, J=8.6 Hz, 1H), 7.32-7.23 (m, 1H), 7.10-7.03 (m, 2H), 6.90 (dd, J=8.7, 10.4 Hz, 2H), 4.08 (dt, J=6.6, 10.8 Hz, 1H), 3.84 (dt, J=4.8, 11.0 Hz, 1H), 3.69-3.61 (m, 2H), 3.02 (br dd, J=8.5, 10.8 Hz, 1H), 2.53 (s, 3H), 2.51 (s, 3H), 2.48-2.39 (m, 2H), 2.05-1.95 (m, 2H), 1.74-1.65 (m, 1H), 1.40 (s, 9H).

### Step 3: 1-[6-[6-[(6-methylpyridazin-3-yl)amino]benzimidazol-1-yl]-2-[rac-trans-2,3,3a,5,6,6a-hexahydro-1H-pyrrolo[3,2-b]pyrrol-4-yl]-3-pyridyl]ethanone

Error! Objects cannot be created from editing field codes.

To a solution of tert-butyl rac-trans-1-[3-acetyl-6-[6-[(6-methylpyridazin-3-yl)amino]benzimidazol-1-yl]-2-pyridyl]-2,3,3a,5,6,6a-hexahydropyrrolo[3,2-b]pyrrole-4-carboxylate (400 mg, 0.72 mmol, 1 equiv.) in DCM (3 mL) was added TFA (3 mL, 12 mmol, 16.64 equiv.). The reaction mixture was stirred at 20° C. for 1 hour. The reaction mixture was concentrated and the residue was dissolved in DCM (20 mL). The pH of the solution was adjusted to 8 with sat. NaHCO₃ aq.sol. and the mixture was extracted with DCM/MeOH 9:1 (9 x). The combined organic layers were dried over Na₂SO₄, filtered and concentrated to afford the title compound (270 mg, 82.4% yield) as a yellow solid. LC-MS: m/z=455.3 [M+H]⁺, ESI pos.

### Step 4: 1-[6-[6-[(6-methylpyridazin-3-yl)amino]benzimidazol-1-yl]-2-[(3aR,6aS)-2,3,3a,5,6,6a-hexahydro-1H-pyrrolo[3,2-b]pyrrol-4-yl]-3-pyridyl]ethanol; formic acid

Error! Objects cannot be created from editing field codes.

To a solution of 1-[6-[6-[(6-methylpyridazin-3-yl)amino]benzimidazol-1-yl]-2-[rac-trans-2,3,3a,5,6,6a-hexahydro-1H-pyrrolo[3,2-b]pyrrol-4-yl]-3-pyridyl]ethanone (250 mg, 0.55 mmol, 1 equiv.) in MeOH (3 mL) was added NaBH₄ (62 mg, 1.65 mmol, 3 equiv.) at 0° C. The mixture was then stirred at 20° C. for 1 hour. NaBH₄ (62.43 mg, 1.65 mmol, 3 equiv.) was added to the solution. The reaction mixture was stirred at 20° C. for another 1 hour. The mixture was diluted with H₂O (30 mL) and extracted with (DCM/MeOH 9:1) (6×30 mL). The combined organics were dried over Na₂SO₄, filtered and concentrated.

The residue was purified by preparative HPLC (Unisil 3-100 C18 Ultra (150 mm×50 mm×10 μm). Flow rate: 25 mL/min. Gradient: 1% to 30% CH₃CN in (0.225% formic acid in H₂O v/v) (10 min) then 100% CH₃CN (2 min)) to give 1-[6-[6-[(6-methylpyridazin-3-yl)amino]benzimidazol-1-yl]-2-[(3aR,6aS)-2,3,3a,5,6,6a-hexahydro-1H-pyrrolo[3,2-b]pyrrol-4-yl]-3-pyridyl]ethanol; formic acid (32 mg, 12.7% yield) as an off-white liophilized solid and 1-[6-[6-[(6-methylpyridazin-3-yl)amino]benzimidazol-1-yl]-2-[(3aS,6aR)-2,3,3a,5,6,6a-hexahydro-1H-pyrrolo[3,2-b]pyrrol-4-yl]-3-pyridyl]ethanol (71.9 mg, 28.1% yield) as a yellow liophilized solid. Regioisomer 1: LC-MS: m/z=457.2 [M+H]⁺, ESI pos. ¹H NMR (400 MHz, DMSO-d₆): δ=9.50 (s, 1H), 9.01 (s, 1H), 8.64 (s, 1H), 8.40 (s, 1H), 8.03 (d, J=8.1 Hz, 1H), 7.67 (d, J=8.6 Hz, 1H), 7.42 (d, J=9.1 Hz, 1H), 7.34 (dd, J=1.8, 8.6 Hz, 1H), 7.27 (d, J=8.1 Hz, 1H), 7.19 (d, J=9.1 Hz, 1H), 5.01 (q, J=6.0 Hz, 1H), 4.40-4.25 (m, 2H), 3.81-3.76 (m, 1H), 3.56-3.48 (m, 3H), 2.49 (s, 3H), 2.30-2.22 (m, 1H), 2.20-2.05 (m, 2H), 1.51-1.39 (m, 1H), 1.32 (d, J=6.3 Hz, 3H). Regiosimer 2: LC-MS: m/z=457.2 [M+H]⁺, ESI pos. ¹H NMR (400 MHz, DMSO-d₆): δ=9.55 (s, 1H), 8.96 (d, J=1.4 Hz, 1H), 8.66 (s, 1H), 8.40 (s, 1H), 7.98 (d, J=8.0 Hz, 1H), 7.67 (d, J=8.8 Hz, 1H), 7.42 (d, J=9.1 Hz, 1H), 7.38 (dd, J=1.7, 8.7 Hz, 1H), 7.33 (d, J=8.0 Hz, 1H), 7.20 (d, J=9.1 Hz, 1H), 4.97 (q, J=6.1 Hz, 1H), 4.43-4.30 (m, 2H), 3.84-3.79 (m, 1H), 3.62-3.51 (m, 3H), 2.49 (s, 3H), 2.30-2.22 (m, 1H), 2.19-2.10 (m, 2H), 1.56-1.46 (m, 4H).

### Example 126

### 1-[6-[6-[(6-Methylpyridazin-3-yl)amino]benzimidazol-1-yl]-2-[(3aS,6aR)-2,3,3a,5,6,6a-hexahydro-1H-pyrrolo[3,2-b]pyrrol-4-yl]-3-pyridyl]ethanol; formic acid

Error! Objects cannot be created from editing field codes.

The title compound 1-[6-[6-[(6-methylpyridazin-3-yl)amino]benzimidazol-1-yl]-2-[(3aS,6aR)-2,3,3a,5,6,6a-hexahydro-1H-pyrrolo[3,2-b]pyrrol-4-yl]-3-pyridyl]ethanol (71.9 mg, 28.1% yield) was obtained in step 4 of example 125 (after the purification by preparative HPLC) as a yellow lyophilized solid. LC-MS: m/z=457.2 [M+H]⁺, ESI pos. ¹H NMR (400 MHz, DMSO-d₆): δ=9.55 (s, 1H), 8.96 (d, J=1.4 Hz, 1H), 8.66 (s, 1H), 8.40 (s, 1H), 7.98 (d, J=8.0 Hz, 1H), 7.67 (d, J=8.8 Hz, 1H), 7.42 (d, J=9.1 Hz, 1H), 7.38 (dd, J=1.7, 8.7 Hz, 1H), 7.33 (d, J=8.0 Hz, 1H), 7.20 (d, J=9.1 Hz, 1H), 4.97 (q, J=6.1 Hz, 1H), 4.43-4.30 (m, 2H), 3.84-3.79 (m, 1H), 3.62-3.51 (m, 3H), 2.49 (s, 3H), 2.30-2.22 (m, 1H), 2.19-2.10 (m, 2H), 1.56-1.46 (m, 4H).

### Example 127

### 1-[6-[5-[(6-Methylpyridazin-3-yl)amino]benzimidazol-1-yl]-2-[rac-trans-2,3,3a,5,6,6a-hexahydro-1H-pyrrolo[3,2-b]pyrrol-4-yl]-3-pyridyl]ethanol

Error! Objects cannot be created from editing field codes.

### Step 1: 1-[6-[5-[(6-methylpyridazin-3-yl)amino]benzimidazol-1-yl]-2-[rac-trans-2,3,3a,5,6,6a-hexahydro-1H-pyrrolo[3,2-b]pyrrol-4-yl]-3-pyridyl]ethanone

Error! Objects cannot be created from editing field codes.

Following the procedure described in step 3 of example 125, with tert-butyl rac-trans-1-[3-acetyl-6-[5-[(6-methylpyridazin-3-yl)amino]benzimidazol-1-yl]-2-pyridyl]-2,3,3a,5,6,6a-hexahydropyrrolo[3,2-b]pyrrole-4-carboxylate (330 mg, 0.59 mmol, 1 equiv.), the title compound (250 mg, 92.4% yield) was obtained as a yellow solid. LC-MS: m/z=455.3 [M+H]⁺, ESI pos.

### Step 2: 1-[6-[5-[(6-methylpyridazin-3-yl)amino]benzimidazol-1-yl]-2-[rac-trans-2,3,3a,5,6,6a-hexahydro-1H-pyrrolo[3,2-b]pyrrol-4-yl]-3-pyridyl]ethanol

Error! Objects cannot be created from editing field codes.

To a solution of 1-[6-[5-[(6-methylpyridazin-3-yl)amino]benzimidazol-1-yl]-2-[rac-trans-2,3,3a,5,6,6a-hexahydro-1H-pyrrolo[3,2-b]pyrrol-4-yl]-3-pyridyl]ethanone (230 mg, 0.51 mmol, 1 equiv.) in MeOH (3 mL) was added NaBH₄ (57 mg, 1.52 mmol, 3 equiv.) at 0° C. The reaction mixture was stirred at 20° C. for 1 hour. DMF (3 mL) and NaBH₄ (57 mg, 1.52 mmol, 3 equiv.) were added to the mixture which was stirred at RT for another 1 hour. The mixture was diluted with H₂O (30 mL) and extracted with (DCM/MeOH 9:1) (6×30 mL). The combined organics were dried over Na₂SO₄, filtered and concentrated.

The residue was purified by preparative HPLC (Unisil 3-100 C18 Ultra (150 mm×50 mm×10 μm). Flow rate: 25 mL/min. Gradient: 1% to 28% CH₃CN in (0.225% formic acid in H₂O v/v) (10 min) then 100% CH₃CN (2 min)). The title compound (137.4 mg, 59.5% yield) as yellow liophilized solid. LC-MS: m/z=457.2 [M+H]⁺, ESI pos. ¹H NMR (400 MHz, DMSO-d₆): δ=9.23 (s, 1H), 8.85-8.79 (m, 1H), 8.40-8.32 (m, 2H), 8.21-8.13 (m, 1H), 8.07-7.98 (m, 1H), 7.59 (td, J=2.4, 8.7 Hz, 1H), 7.48-7.37 (m, 1H), 7.34 (d, J=9.0 Hz, 1H), 7.10 (d, J=9.0 Hz, 1H), 5.05-4.89 (m, 1H), 4.32-4.24 (m, 1H), 4.06 (dt, J=5.9, 10.7 Hz, 1H), 3.76-3.70 (m, 2H), 3.65-3.49 (m, 2H), 2.49 (s, 3H), 2.31-2.22 (m, 1H), 2.14-2.02 (m, 2H), 1.61-1.25 (m, 4H).

### Example 128

### 1-[2-[3-(Difluoromethyl)-5-methyl-pyrazol-1-yl]-6-[6-[(6-methylpyridazin-3-yl)amino]benzimidazol-1-yl]-3-pyridyl]ethanol

Error! Objects cannot be created from editing field codes.

### Step 1: 1-[6-chloro-2-[3-(difluoromethyl)-5-methyl-pyrazol-1-yl]-3-pyridyl]ethanone and 1-[6-chloro-2-[5-(difluoromethyl)-3-methyl-pyrazol-1-yl]-3-pyridyl]ethanone

Error! Objects cannot be created from editing field codes.

To a solution of 1-(6-chloro-2-fluoro-3-pyridyl)ethanone (CAS #1260663-13-5, 1.0 g, 5.76 mmol, 1 equiv.) and 3-(difluoromethyl)-5-methyl-1H-pyrazole (837.3 mg, 6.34 mmol, 1.1 equiv.) in DMSO (38 mL) was added K₂CO₃ (1.19 g, 8.64 mmol, 1.5 equiv.). The reaction mixture was stirred at 65° C. for 2 hours. The mixture was poured into H₂O (50 mL) and extracted with EtOAc (3×50 mL). The combined organic layers were washed with brine (3×50 mL) dried over anhydrous Na₂SO₄, filtered and concentrated under vacuum. The residue was purified by flash chromatography (silica gel, 10% EtOAc in PE) to give 1-[6-chloro-2-[3-(difluoromethyl)-5-methyl-pyrazol-1-yl]-3-pyridyl]ethanone (885 mg, 3.09 mmol, 53.7% yield) as a white solid and 1-[6-chloro-2-[5-(difluoromethyl)-3-methyl-pyrazol-1-yl]-3-pyridyl]ethanone (200 mg, 0.662 mmol, 11.5% yield) as a white solid. Regiosimore 1: LC-MS: m/z=286.1 [M+H]⁺, ESI pos. ¹H NMR (400 MHz, CDCl₃): δ=7.96-7.91 (m, 1H), 7.46-7.40 (m, 1H), 6.77-6.47 (m, 2H), 2.65-2.61 (m, 3H), 2.07-2.01 (m, 3H). Regioisomer 2: LC-MS: m/z=286.1 [M+H]⁺, ESI pos. ¹H NMR (600 MHz, DMSO-d6) δ ppm 8.00-8.06 (m, 1H), 7.90-7.96 (m, 1H), 7.37-7.58 (m, 2H), 6.66 (br s, 1H), 2.25 (s, 3H), 2.13 (s, 3H).

### Step 2: 1-[2-[3-(difluoromethyl)-5-methyl-pyrazol-1-yl]-6-[5-[(6-methylpyridazin-3-yl)amino]benzimidazol-1-yl]-3-pyridyl]ethanone and 1-[2-[3-(difluoromethyl)-5-methyl-pyrazol-1-yl]-6-[6-[(6-methylpyridazin-3-yl)amino]benzimidazol-1-yl]-3-pyridyl]ethanone

Error! Objects cannot be created from editing field codes.

Following the procedure described in step 4 of example 113, with a reaction time of 16 h at 50° C., the residue obtained after extraction work-up was purified by preparative NPLC (Welch Ultimate XB-CN (250 mm×50 mm×10 μm). Flow rate: 140 mL/min. Gradient: 40% to 80% (EtOH with 0.1% NH₄OH v/v) in hexane (12 min) then 100% EtOH (5 min)) to give 1-[2-[3-(difluoromethyl)-5-methyl-pyrazol-1-yl]-6-[5-[(6-methylpyridazin-3-yl)amino]benzimidazol-1-yl]-3-pyridyl]ethanone (300 mg, 22.6% yield) as a light yellow solid (containing traces of 1-[2-[5-(difluoromethyl)-3-methyl-pyrazol-1-yl]-6-[5-[(6-methylpyridazin-3-yl)amino]benzimidazol-1-yl]-3-pyridyl]ethanone) and 1-[2-[3-(difluoromethyl)-5-methyl-pyrazol-1-yl]-6-[6-[(6-methylpyridazin-3-yl)amino]benzimidazol-1-yl]-3-pyridyl]ethanone (300 mg, 22.6% yield) as a light yellow solid. Regioisomer 1: LC-MS: m/z=475.1 [M+H]⁺, ESI pos. ¹H NMR (400 MHz, DMSO-d₆): δ=9.29 (s, 1H), 9.09 (s, 1H), 8.46 (d, J=1.8 Hz, 1H), 8.39 (d, J=8.4 Hz, 1H), 8.21 (d, J=8.4 Hz, 1H), 8.17 (d, J=8.8 Hz, 1H), 7.54 (dd, J=2.0, 8.9 Hz, 1H), 7.35 (d, J=9.0 Hz, 1H), 7.11 (d, J=9.2 Hz, 1H), 7.05 (s, 1H), 6.75 (s, 1H), 2.61 (s, 3H), 2.49 (br s, 3H), 2.01 (s, 3H). Regioisomer 2: LC-MS: m/z=475.1 [M+H]⁺, ESI pos. ¹H NMR (400 MHz, DMSO-d₆): δ=9.31 (s, 1H), 8.97 (s, 1H), 8.86 (d, J=1.8 Hz, 1H), 8.40 (d, J=8.3 Hz, 1H), 8.16 (d, J=8.3 Hz, 1H), 7.71 (d, J=8.7 Hz, 1H), 7.53 (dd, J=2.0, 8.7 Hz, 1H), 7.30 (d, J=9.2 Hz, 1H), 7.08 (d, J=9.0 Hz, 1H), 7.03 (s, 1H), 6.69 (s, 1H), 2.60 (s, 3H), 2.47 (s, 3H), 2.00 (s, 3H).

### Step 3: 1-[2-[3-(difluoromethyl)-5-methyl-pyrazol-1-yl]-6-[6-[(6-methylpyridazin-3-yl)amino]benzimidazol-1-yl]-3-pyridyl]ethanol

Error! Objects cannot be created from editing field codes.

Following the procedure described in step 3 of example 107, the title compound (179.2 mg, 61.5% yield) was obtained as a light yellow liophilized solid. Purification by preparative HPLC (Phenomenex Synergi C18 (150 mm×25 mm×10 μm). Flow rate: 25 mL/min. Gradient: 8% to 38% CH₃CN in (0.225% formic acid in H₂O v/v) (10 min) then 100% CH₃CN (2 min)). 477.2 [M+H]⁺, ESI pos. ¹H NMR (400 MHz, CD₃OD): δ=8.79 (s, 1H), 8.71 (d, J=1.7 Hz, 1H), 8.53 (d, J=8.4 Hz, 1H), 8.10 (d, J=8.4 Hz, 1H), 7.69 (d, J=8.7 Hz, 1H), 7.45 (dd, J=2.0, 8.7 Hz, 1H), 7.35 (d, J=9.2 Hz, 1H), 7.16 (d, J=9.2 Hz, 1H), 6.93-6.63 (m, 1H), 6.53 (s, 1H), 4.83 (br s, 1H), 2.55 (s, 3H), 2.38 (s, 3H), 1.39 (d, J=6.5 Hz, 3H).

### Example 129

### 1-[2-[3-(Difluoromethyl)-5-methyl-pyrazol-1-yl]-6-[5-[(6-methylpyridazin-3-yl)amino]benzimidazol-1-yl]-3-pyridyl]ethanol; formic acid

Error! Objects cannot be created from editing field codes.

Following the procedure described in step 3 of example 107, starting with 1-[2-[3-(difluoromethyl)-5-methyl-pyrazol-1-yl]-6-[5-[(6-methylpyridazin-3-yl)amino]benzimidazol-1-yl]-3-pyridyl]ethanone (obtained in step 2 of example 128) (290 mg, 0.61 mmol, 1 equiv.) the title compound (127 mg, 43.9% yield) was obtained as a light yellow liophilized solid. Purification by preparative HPLC (column Phenomenex Synergi C18 (150 mm×25 mm×10 μm). Flow rate: 25 mL/min. Gradient: 9% to 39% CH₃CN in (0.225% formic acid in H₂O v/v) (10 min) then 100% CH₃CN (2 min)). 477.4 [M+H]⁺, ESI pos. ¹H NMR (400 MHz, CD₃OD): δ=8.90 (s, 1H), 8.47 (d, J=8.4 Hz, 1H), 8.21 (d, J=1.6 Hz, 1H), 8.15-8.12 (m, 1H), 8.09 (d, J=8.4 Hz, 1H), 7.57 (br d, J=8.8 Hz, 1H), 7.40 (d, J=9.2 Hz, 1H), 7.18 (d, J=9.2 Hz, 1H), 6.95-6.65 (m, 1H), 6.58 (s, 1H), 4.83 (q, J=6.5 Hz, 1H), 2.54 (s, 3H), 2.40 (s, 3H), 1.39 (d, J=6.5 Hz, 3H).

### Example 130

### 1-[6-[6-Bromo-5-[4-(oxetan-3-yl)piperazin-1-yl]benzimidazol-1-yl]-3-(1-hydroxyethyl)-2-pyridyl]-5-methyl-pyrazole-3-carbonitrile

Error! Objects cannot be created from editing field codes.

### Step 1: 4-bromo-2-nitro-5-[4-(oxetan-3-yl)piperazin-1-yl]aniline

Error! Objects cannot be created from editing field codes.

To a solution of 4-bromo-5-fluoro-2-nitro-aniline (0.95 g, 4.04 mmol, 1.0 equiv.) and 1-(oxetan-3-yl)piperazine (0.86 g, 6.06 mmol, 1.5 equiv.) in DMSO (10 mL) was added TEA (1.69 mL, 12.13 mmol, 3.0 equiv.). The reaction mixture was stirred at 50° C. for 16 hours. The reaction mixture was diluted with water and extracted with EtOAc. The organic layers were washed with brine, dried over Na₂SO₄ and concentrated under vacuum to give 4-bromo-2-nitro-5-[4-(oxetan-3-yl)piperazin-1-yl]aniline (1.3 g, 3.64 mmol, 90.0% yield) as yellow solid. LC-MS: m/z=357.1 [M+H]⁺, ESI pos.

### Step 2: 4-bromo-5-[4-(oxetan-3-yl)piperazin-1-yl]benzene-1,2-diamine

Error! Objects cannot be created from editing field codes.

To a solution of 4-bromo-2-nitro-5-[4-(oxetan-3-yl)piperazin-1-yl]aniline (1.0 g, 2.8 mmol, 1.0 equiv.) in EtOH (18 mL) was added Fe⁰ (781.6 mg, 14 mmol, 5.0 equiv.), NH₄Cl (1.5 g, 28 mmol, 10 equiv.) and water (6 mL). The reaction mixture was stirred under notrogen at 50° C. for 12 hours. The reaction mixture was filtered, the filtrate was concentrated under reduced pressure. The residue was purified by reversed phase chromatography (50% CH₃CN in H₂O) to yield 4-bromo-5-[4-(oxetan-3-yl)piperazin-1-yl]benzene-1,2-diamine (500 mg, 1.53 mmol, 54.6% yield) as yellow solid. LC-MS: m/z=329.0 [M+H]⁺, ESI pos.

### Step 3: 5-bromo-6-[4-(oxetan-3-yl)piperazin-1-yl]-1H-benzimidazole

Error! Objects cannot be created from editing field codes.

To a solution of 4-bromo-5-[4-(oxetan-3-yl)piperazin-1-yl]benzene-1,2-diamine (400.0 mg, 1.22 mmol, 1.0 equiv.) in EtOH (8 mL) was added trimethoxymethane (1.30 g, 12.22 mmol, 10 equiv.) and TsOH (21.1 mg, 0.120 mmol, 0.10 equiv.), and the mixture was stirred at 80° C. for 1 hour. Sat. aq. NaHCO₃ was added to the reaction to adjust pH to 7-8, and the product was extracted with EtOAc. The combined organic layers were concentrated under vacuum to yield 5-bromo-6-[4-(oxetan-3-yl)piperazin-1-yl]-1H-benzimidazole (400 mg, 1.19 mmol, 97.0% yield) as orange solid. LC-MS: m/z=339.0 [M+H]⁺, ESI pos.

### Step 4:1-[3-acetyl-6-[6-bromo-5-[4-(oxetan-3-yl)piperazin-1-yl]benzimidazol-1-yl]-2-pyridyl]-5-methyl-pyrazole-3-carbonitrile and 1-[3-acetyl-6-[5-bromo-6-[4-(oxetan-3-yl)piperazin-1-yl]benzimidazol-1-yl]-2-pyridyl]-5-methyl-pyrazole-3-carbonitrile

Error! Objects cannot be created from editing field codes.

Prepared in analogy to example 64, step 2 using 5-bromo-6-[4-(oxetan-3-yl)piperazin-1-yl]-1H-benzimidazole (400.0 mg, 1.19 mmol, 1.0 equiv.) and 1-(3-acetyl-6-chloro-2-pyridyl)-5-methyl-pyrazole-3-carbonitrile (309.22 mg, 1.19 mmol, 1.0 equiv., prepared in example 64, step 1). The regioisomers were purified by preparative TLC (10% MeOH in DCM) to give 1-[3-acetyl-6-[6-bromo-5-[4-(oxetan-3-yl)piperazin-1-yl]benzimidazol-1-yl]-2-pyridyl]-5-methyl-pyrazole-3-carbonitrile (230 mg, 0.410 mmol, 32.81% yield) as yellow solid and 1-[3-acetyl-6-[5-bromo-6-[4-(oxetan-3-yl)piperazin-1-yl]benzimidazol-1-yl]-2-pyridyl]-5-methyl-pyrazole-3-carbonitrile (240.9 mg, 0.430 mmol, 34% yield) as orange solid. Regioisomer 1: LC-MS: m/z=563.1 [M+H]⁺, ESI pos. ¹H NMR (400 MHz, DMSO-d₆) δ=9.13 (s, 1H), 8.60 (d, J=8.4 Hz, 1H), 8.32 (d, J=8.4 Hz, 1H), 8.06 (d, J=11.2 Hz, 2H), 7.16 (s, 1H), 4.60-4.54 (m, 2H), 4.49 (t, J=6.1 Hz, 2H), 3.50 (t, J=6.4 Hz, 1H), 2.98 (br s, 4H), 2.49-2.49 (m, 3H), 2.47 (br s, 4H), 2.28 (s, 3H). Regioisomer 2: LC-MS: m/z=563.1 [M+H]⁺, ESI pos. ¹H NMR (400 MHz, DMO-d₆) 6=9.19 (s, 1H), 8.55 (d, J=8.6 Hz, 1H), 8.51 (s, 1H), 8.32 (d, J=8.6 Hz, 1H), 7.62 (s, 1H), 7.16 (d, J=0.6 Hz, 1H), 4.59-4.55 (m, 2H), 4.51-4.46 (m, 2H), 3.50 (quin, J=6.4 Hz, 1H), 3.03 (br s, 4H), 2.56 (s, 3H), 2.49-2.45 (m, 4H), 2.21 (s, 3H).

### Step 5: 1-[6-[6-bromo-5-[4-(oxetan-3-yl)piperazin-1-yl]benzimidazol-1-yl]-3-(-hydroxyethyl)-2-pyridyl]-5-methyl-pyrazole-3-carbonitrile

Error! Objects cannot be created from editing field codes.

Prepared in analogy to example 53, step 4 using 1-[3-acetyl-6-[6-bromo-5-[4-(oxetan-3-yl)piperazin-1-yl]benzimidazol-1-yl]-2-pyridyl]-5-methyl-pyrazole-3-carbonitrile (200.0 mg, 0.360 mmol, 1.0 equiv.) and NaBH₄ (40.0 mg, 1.06 mmol, 3.0 equiv.) in MeOH/THF to yield 1-[6-[6-bromo-5-[4-(oxetan-3-yl)piperazin-1-yl]benzimidazol-1-yl]-3-(1-hydroxyethyl)-2-pyridyl]-5-methyl-pyrazole-3-carbonitrile (143.8 mg, 0.260 mmol, 65.6% yield) as orange solid. LC-MS: m/z=565.1 [M+H]⁺, ESI pos.

### Example 133

### 1-[2-(3-Ethoxy-5-methyl-pyrazol-1-yl)-6-[5-[(6-methylpyridazin-3-yl)amino]benzimidazol-1-yl]-3-pyridyl]ethanol; formic acid

Error! Objects cannot be created from editing field codes.

### Step 1: 1-[6-chloro-2-(3-ethoxy-5-methyl-pyrazol-1-yl)-3-pyridyl]ethanone

Error! Objects cannot be created from editing field codes.

Prepared in analogy to example 64, step 1 using 3-ethoxy-5-methyl-1H-pyrazole (420.0 mg, 3.33 mmol, 1.0 equiv.) and 1-(6-chloro-2-fluoro-3-pyridyl)ethanone (CAS #1260663-13-5, 866.8 mg, 4.99 mmol, 1.5 equiv.) to yield 1-[6-chloro-2-(3-ethoxy-5-methyl-pyrazol-1-yl)-3-pyridyl]ethanone (110 mg, 0.390 mmol, 11.8% yield) as orange solid. LC-MS: m/z=280.1 [M+H]⁺, ESI pos.

### Step 2: 1-[2-(3-ethoxy-5-methyl-pyrazol-1-yl)-6-[5-[(6-methylpyridazin-3-yl)amino]benzimidazol-1-yl]-3-pyridyl]ethanone and 1-[2-(3-ethoxy-5-methyl-pyrazol-1-yl)-6-[6-[(6-methylpyridazin-3-yl)amino]benzimidazol-1-yl]-3-pyridyl]ethanone

Error! Objects cannot be created from editing field codes.

Prepared in analogy to example 64, step 2 using 1-[6-chloro-2-(3-ethoxy-5-methyl-pyrazol-1-yl)-3-pyridyl]ethanone (110.0 mg, 0.390 mmol, 1.0 equiv.) and N-(6-methylpyridazin-3-yl)-1H-benzimidazol-5-amine (106.3 mg, 0.470 mmol, 1.2 equiv.) to yield 1-[2-(3-ethoxy-5-methyl-pyrazol-1-yl)-6-[5-[(6-methylpyridazin-3-yl)amino]benzimidazol-1-yl]-3-pyridyl]ethanone (90 mg, 0.190 mmol, 48.85% yield) as yellow gum. Regioisomer 1: LC-MS:469.2 [M+H]⁺, ESI pos. ¹H NMR (400 MHz, CD₃OD) δ=8.82 (s, 1H), 8.21 (d, J=1.8 Hz, 1H), 8.14 (d, J=8.3 Hz, 1H), 8.09 (d, J=8.9 Hz, 1H), 7.79 (d, J=8.2 Hz, 1H), 7.60 (dd, J=2.1, 8.8 Hz, 1H), 7.35 (d, J=9.2 Hz, 1H), 7.12 (d, J=9.2 Hz, 1H), 5.87 (d, J=0.6 Hz, 1H), 4.16 (q, J=7.0 Hz, 2H), 2.60 (s, 3H), 2.53 (s, 3H), 2.17 (s, 3H), 1.37 (t, J=7.0 Hz, 3H). Regioisomer 2: LC-MS:469.2 [M+H]⁺, ESI pos.

### Step 3: 1-[2-(3-ethoxy-5-methyl-pyrazol-1-yl)-6-[5-[(6-methylpyridazin-3-yl)amino]benzimidazol-1-yl]-3-pyridyl]ethanol; formic acid

Error! Objects cannot be created from editing field codes.

Prepared in analogy to example 53, step 4 using 1-[2-(3-ethoxy-5-methyl-pyrazol-1-yl)-6-[5-[(6-methylpyridazin-3-yl)amino]benzimidazol-1-yl]-3-pyridyl]ethanone (90.0 mg, 0.190 mmol, 1.0 equiv.) and NaBH₄ (21.8 mg, 0.580 mmol, 3.0 equiv.) to yield 1-[2-(3-ethoxy-5-methyl-pyrazol-1-yl)-6-[5-[(6-methylpyridazin-3-yl)amino]benzimidazol-1-yl]-3-pyridyl]ethanol; formic acid (42.3 mg, 0.080 mmol, 46.8% yield) as yellow solid. LC-MS: m/z=471.2 [M+H]⁺, ESI pos. ¹H NMR (400 MHz, DMSO-d₆) δ=9.21 (s, 1H), 8.96 (s, 1H), 8.42 (d, J=1.9 Hz, 1H), 8.35 (d, J=8.5 Hz, 1H), 8.18 (s, 1H), 8.08 (dd, J=6.4, 8.6 Hz, 2H), 7.50 (dd, J=2.1, 8.9 Hz, 1H), 7.33 (d, J=9.1 Hz, 1H), 7.08 (d, J=9.1 Hz, 1H), 5.86 (s, 1H), 5.66-5.17 (m, 1H), 4.96 (br d, J=6.4 Hz, 1H), 4.16 (q, J=7.0 Hz, 2H), 2.48 (s, 3H), 2.31 (s, 3H), 1.33 (t, J=7.0 Hz, 3H), 1.29 (d, J=6.4 Hz, 3H).

### Example 134

### 1-[2-(3-Ethoxy-5-methyl-pyrazol-1-yl)-6-[6-[(6-methylpyridazin-3-yl)amino]benzimidazol-1-yl]-3-pyridyl]ethanol

Error! Objects cannot be created from editing field codes.

Prepared in analogy to example 53, step 4 using 1-[2-(3-ethoxy-5-methyl-pyrazol-1-yl)-6-[6-[(6-methylpyridazin-3-yl)amino]benzimidazol-1-yl]-3-pyridyl]ethanone (90.0 mg, 0.190 mmol, 1.0 equiv., prepared in example 133, step 2) and NaBH₄ (21.8 mg, 0.580 mmol, 3 equivalents to yield 1-[2-(3-ethoxy-5-methyl-pyrazol-1-yl)-6-[6-[(6-methylpyridazin-3-yl)amino]benzimidazol-1-yl]-3-pyridyl]ethanol (52.2 mg, 0.110 mmol, 57.8% yield) as yellow solid. LC-MS: m/z=471.2 [M+H]⁺, ESI pos. ¹H NMR (400 MHz, DMSO-d₆) δ=9.28 (s, 1H), 8.78 (s, 1H), 8.65 (d, J=1.9 Hz, 1H), 8.39 (d, J=8.4 Hz, 1H), 7.99 (d, J=8.3 Hz, 1H), 7.69 (d, J=8.6 Hz, 1H), 7.47 (dd, J=2.1, 8.7 Hz, 1H), 7.30 (d, J=9.1 Hz, 1H), 7.08 (d, J=9.0 Hz, 1H), 5.80 (d, J=0.6 Hz, 1H), 5.43 (br s, 1H), 4.95 (br d, J=6.4 Hz, 1H), 4.14 (q, J=7.0 Hz, 2H), 2.47 (s, 3H), 2.30 (s, 3H), 1.34-1.28 (m, 6H).

### Example 135

### 1-[2-(6-Methylpyridazin-4-yl)-6-[5-[(6-methylpyridazin-3-yl)amino]benzimidazol-1-yl]-3-pyridyl]ethanol

Error! Objects cannot be created from editing field codes.

### Step 1: 1-[2-(6-methylpyridazin-4-yl)-6-[5-[(6-methylpyridazin-3-yl)amino]benzimidazol-1-yl]-3-pyridyl]ethanone

Error! Objects cannot be created from editing field codes.

1-[2-chloro-6-[5-[(6-methylpyridazin-3-yl)amino]benzimidazol-1-yl]-3-pyridyl]ethanone (160.0 mg, 0.420 mmol, 1.0 equiv.), 3-methylpyridazine-5-boronic acid, pinacol ester (111.54 mg, 0.510 mmol, 1.2 equiv.), PdCl₂(dppf) CH₂Cl₂ (34.3 mg, 0.042 mmol, 0.1 equiv.) and Na₂CO₃ (89 mg, 0.84 mmol, 2 equiv.) were suspended in 1,4-dioxane (1.33 mL) and H₂O (0.33 mL). The reaction mixture was heated to 80° C. and stirred for 12 hours. The mixture was cooled to RT, diluted with H₂O (7 mL) and EtOAc (7 mL) and stirred for 30 minutes. The insoluble materials were filtered off and the filter cake was washed with 7 mL of EtOAc. The organic layer from the filtrate was washed with brine, dried over anhydrous Na₂SO₄, filtered and concentrated to leave the crude title compound to yield 1-[2-(6-methylpyridazin-4-yl)-6-[5-[(6-methylpyridazin-3-yl)amino]benzimidazol-1-yl]-3-pyridyl]ethanone (140 mg, 0.320 mmol, 75.9% yield) as yellow solid. LC-MS: m/z=437.1 [M+H]⁺, ESI pos.

### Step 2: 1-[2-(6-methylpyridazin-4-yl)-6-[5-[(6-methylpyridazin-3-yl)amino]benzimidazol-1-yl]-3-pyridyl]ethanol

Error! Objects cannot be created from editing field codes.

Prepared in analogy to example 53, step 4 using 1-[2-(6-methylpyridazin-4-yl)-6-[5-[(6-methylpyridazin-3-yl)amino]benzimidazol-1-yl]-3-pyridyl]ethanone (140.0 mg, 0.320 mmol, 1.0 equiv.) and NaBH₄ in MeOH/DMF (1:1) (36.4 mg, 0.960 mmol, 3.0 equiv.) at −40° C. to yield 1-[2-(6-methylpyridazin-4-yl)-6-[5-[(6-methylpyridazin-3-yl)amino]benzimidazol-1-yl]-3-pyridyl]ethanol (22 mg, 0.050 mmol, 15.6% yield) as yellow solid. LC-MS: m/z=439.1 [M+H]⁺, ESI pos. ¹H NMR (400 MHz, CD₃OD) δ=9.35 (d, J=1.7 Hz, 1H), 8.90 (s, 1H), 8.38 (d, J=8.6 Hz, 1H), 8.20-8.15 (m, 2H), 8.01 (d, J=8.6 Hz, 1H), 7.92 (d, J=2.0 Hz, 1H), 7.53 (dd, J=2.0, 8.9 Hz, 1H), 7.45 (d, J=9.2 Hz, 1H), 7.23 (d, J=9.2 Hz, 1H), 4.98 (q, J=6.4 Hz, 1H), 2.82 (s, 3H), 2.55 (s, 3H), 1.51 (d, J=6.4 Hz, 3H).

### Example 136

### 4-[3-(1-Hydroxyethyl)-6-[5-[(6-methylpyridazin-3-yl)amino]benzimidazol-1-yl]-2-pyridyl]-1-(2,2,2-trifluoroethyl)piperazin-2-one

Error! Objects cannot be created from editing field codes.

### Step 1: benzyl 3-oxo-4-(2,2,2-trifluoroethyl)piperazine-1-carboxylate

Error! Objects cannot be created from editing field codes.

To a mixture of 2,2,2-trifluoroethyl trifluoromethanesulfonate (3.3 g, 14.09 mmol, 1.1 equiv.) in anhydrous DMF (60 mL) was added NaH (618.0 mg, 15.45 mmol, 1.2 equiv.) under nitrogen at 0° C. and stirred at 0° C. for 30 min. Benzyl 3-oxopiperazine-1-carboxylate (3.0 g, 12.81 mmol, 1.0 equiv.) was added dropwise and the reaction was stirred at RT for 16 hours. The reaction mixture was quenched with aq. sat. NH₄Cl and extracted with EtOAc. The combined organic layers were concentrated under reduced pressure and purified by preparative HPLC (Phenomenex Luna C18 250×80 mm×10 μm, gradient 35-65% CH₃CN in H₂O (with 0.225% formic acid) over 20 min, then 100% CH₃CN (4 min), flow rate 140 mL/min, 1 injection), to yield benzyl 3-oxo-4-(2,2,2-trifluoroethyl)piperazine-1-carboxylate (1700 mg, 5.37 mmol, 41.97% yield) as colorless oil. LC-MS: m/z=317.2 [M+H]⁺, ESI pos.

### Step 2: 1-(2,2,2-trifluoroethyl)piperazin-2-one

Error! Objects cannot be created from editing field codes.

A mixture of benzyl 3-oxo-4-(2,2,2-trifluoroethyl)piperazine-1-carboxylate (1.7 g, 5.37 mmol, 1.0 equiv.) and Pd/C (578.0 mg, 0.540 mmol, 0.10 equiv.) in MeOH (30 mL) was hydrogenated under 45 psi of H₂ pressure at 30° C. for 16 hours. The suspension was filtered through a pad of Celite and the filter cake was washed with MeOH. The combined filtrates were concentrated to give 1-(2,2,2-trifluoroethyl)piperazin-2-one (100 mg, 0.550 mmol, 86.8% yield) as dark green oil. LC-MS: m/z=183.0 [M+H]⁺, ESI pos.

### Step 3: 4-[3-acetyl-6-[6-[(6-methylpyridazin-3-yl)amino]benzimidazol-1-yl]-2-pyridyl]-1-(2,2,2-trifluoroethyl)piperazin-2-one and 4-[3-acetyl-6-[5-[(6-methylpyridazin-3-yl)amino]benzimidazol-1-yl]-2-pyridyl]-1-(2,2,2-trifluoroethyl)piperazin-2-one

Error! Objects cannot be created from editing field codes.

A solution of a mixture of 1-[2-chloro-6-[5-[(6-methylpyridazin-3-yl)amino]benzimidazol-1-yl]-3-pyridyl]ethanone and 1-[2-chloro-6-[6-[(6-methylpyridazin-3-yl)amino]benzimidazol-1-yl]-3-pyridyl]ethanone (200.0 mg, 0.530 mmol, 1 equiv.), 1-(2,2,2-trifluoroethyl)piperazin-2-one (164.0 mg, 0.9 mmol, 1.7 equiv.) and DIPEA (205.0 mg, 1.59 mmol, 3.0 equiv.) in DMF (3 mL) was stirred at 30° C. for 16 hours. The reaction mixture was quenched with water and extracted with EtOAc. The combined organic layers were concentrated under reduced pressure and purified by preparative NPLC (Welch Ultimate XB-SiOH 250 mm×50 mm×10 μm, gradient 20-60% EtOH in hexane over 15 min, then 100% EtOH (3 min), flow rate 100 mL/min) to give 4-[3-acetyl-6-[5-[(6-methylpyridazin-3-yl)amino]benzimidazol-1-yl]-2-pyridyl]-1-(2,2,2-trifluoroethyl)piperazin-2-one (150 mg, 0.290 mmol, 54.2% yield) as light yellow solid and 4-[3-acetyl-6-[5-[(6-methylpyridazin-3-yl)amino]benzimidazol-1-yl]-2-pyridyl]-1-(2,2,2-trifluoroethyl)piperazin-2-one (50 mg, 0.100 mmol, 18.1% yield) as light yellow solid. Regioisomer 1: LC-MS: m/z=525.4 [M+H]⁺, ESI pos. ¹H NMR (400 MHz, CDCl₃) δ=8.60 (s, 1H), 8.21 (d, J=8.3 Hz, 1H), 8.03 (d, J=8.9 Hz, 1H), 7.78 (d, J=1.8 Hz, 1H), 7.44 (dd, J=1.9, 8.8 Hz, 1H), 7.21 (s, 2H), 7.10 (d, J=8.3 Hz, 1H), 4.18-4.08 (m, 5H), 3.90-3.84 (m, 2H), 3.78-3.74 (m, 2H), 2.66 (s, 3H), 2.61 (s, 3H). Regioisomer 2: LC-MS: m/z=525.4 [M+H]⁺, ESI pos.

### Step 4: 4-[3-(1-hydroxyethyl)-6-[5-[(6-methylpyridazin-3-yl)amino]benzimidazol-1-yl]-2-pyridyl]-1-(2,2,2-trifluoroethyl)piperazin-2-one

Error! Objects cannot be created from editing field codes.

Prepared in analogy to example 53, step 4 using 4-[3-acetyl-6-[5-[(6-methylpyridazin-3-yl)amino]benzimidazol-1-yl]-2-pyridyl]-1-(2,2,2-trifluoroethyl)piperazin-2-one (50 mg, 0.100 mmol, 1.0 equiv.) and NaBH₄ (8.7 mg, 0.231 mmol, 3.0 equiv.) in DCM/MeOH (9:1) to yield 4-[3-(1-hydroxyethyl)-6-[5-[(6-methylpyridazin-3-yl)amino]benzimidazol-1-yl]-2-pyridyl]-1-(2,2,2-trifluoroethyl)piperazin-2-one (3.3 mg, 0.006 mmol, 8.22% yield) as light yellow solid. LC-MS: m/z=527.4 [M+H]⁺, ESI pos. ¹H NMR (400 MHz, CD₃OD) δ=8.78 (s, 1H), 8.19 (d, J=1.8 Hz, 1H), 8.17 (d, J=8.2 Hz, 1H), 8.12 (d, J=8.8 Hz, 1H), 7.60 (d, J=4.5 Hz, 1H), 7.60-7.57 (m, 1H), 7.38 (d, J=9.2 Hz, 1H), 7.17 (d, J=9.2 Hz, 1H), 5.21 (q, J=6.4 Hz, 1H), 4.22 (d, J=9.3 Hz, 2H), 4.17 (d, J=17.0 Hz, 1H), 4.07-4.02 (m, 1H), 3.78-3.71 (m, 1H), 3.68-3.59 (m, 3H), 2.54 (s, 3H), 1.55 (d, J=6.5 Hz, 3H).

### Example 137

### 4-[3-(1-Hydroxyethyl)-6-[6-[(6-methylpyridazin-3-yl)amino]benzimidazol-1-yl]-2-pyridyl]-1-(2,2,2-trifluoroethyl)piperazin-2-one

Error! Objects cannot be created from editing field codes.

Prepared in analogy to example 53, step 4 using 4-[3-acetyl-6-[6-[(6-methylpyridazin-3-yl)amino]benzimidazol-1-yl]-2-pyridyl]-1-(2,2,2-trifluoroethyl)piperazin-2-one (40.0 mg, 0.080 mmol, 1.0 equiv., prepared in example 136, step 3) and NaBH₄ (9.0 mg, 0.240 mmol, 3.12 equiv.) in DCM/MeOH (9:1) to yield 4-[3-(1-hydroxyethyl)-6-[6-[(6-methylpyridazin-3-yl)amino]benzimidazol-1-yl]-2-pyridyl]-1-(2,2,2-trifluoroethyl)piperazin-2-one (2.5 mg, 0.005 mmol, 6.23% yield) as light yellow solid. LC-MS: m/z=527.4 [M+H]⁺, ESI pos. ¹H NMR (400 MHz, CD₃OD) δ=9.04 (d, J=2.0 Hz, 1H), 8.71 (s, 1H), 8.20 (d, J=8.2 Hz, 1H), 7.64 (dd, J=8.5, 16.4 Hz, 2H), 7.43-7.32 (m, 2H), 7.15 (d, J=9.2 Hz, 1H), 5.21 (q, J=6.6 Hz, 1H), 4.24-4.11 (m, 3H), 4.07-4.00 (m, 1H), 3.82-3.75 (m, 1H), 3.72-3.59 (m, 3H), 2.53 (s, 3H), 1.56 (d, J=6.5 Hz, 3H).

### Example 140

### 1-[3-(1-Hydroxyethyl)-6-[5-methoxy-6-[(6-methylpyridazin-3-yl)amino]benzimidazol-1-yl]-2-pyridyl]-5-methyl-pyrazole-3-carbonitrile

Error! Objects cannot be created from editing field codes.

### Step 1: N-(2-methoxy-4,5-dinitro-phenyl)-6-methyl-pyridazin-3-amine

Error! Objects cannot be created from editing field codes.

To a solution of potassium tert-butoxide (488.06 mg, 4.35 mmol, 2.0 equiv.) in DMSO (5 mL) was added 3-amino-6-methylpyridazine (237.33 mg, 2.17 mmol, 1.0 equiv.) and 1-fluoro-2-methoxy-4,5-dinitro-benzene (470.0 mg, 2.17 mmol, 1.0 equiv.). Then the brown suspension was stirred at 0° C. for 3 hours. The mixture was purified by silica column (water (0.05% ammonium hydroxide v/v)-CH₃CN) (100/0˜7/94) to yield N-(2-methoxy-4,5-dinitro-phenyl)-6-methyl-pyridazin-3-amine (560 mg, 1.83 mmol, 84.36% yield) as light brown solid. LC-MS: m/z=306.0 [M+H]⁺, ESI pos.

### Step 2: 6-methoxy-N-(6-methylpyridazin-3-yl)-1H-benzimidazol-5-amine

Error! Objects cannot be created from editing field codes.

To a solution of N-(2-methoxy-4,5-dinitro-phenyl)-6-methyl-pyridazin-3-amine (800.0 mg, 2.62 mmol, 1.0 equiv.) in formic acid (120.62 mg, 2.62 mmol, 1.0 equiv.) was added nickel (101.77 mg, 1.73 mmol, 0.66 equiv.). The mixture was stirred at 30° C. for 16 h under H₂ (45 psi). The mixture was filtered and the filtrate was concentrated in vacuo. The resulting residue was treated with MeOH (5 mL), the liquid was collected and concentrated in vacuo. The brown solid was purified by preparative HPLC (Waters Xbridge C18 150 mm×50 mm×10 μm, gradient 3-33% CH₃CN in H₂O (with 10 mM NH₄CO₃) over 11 min, then 100% CH₃CN (2 min), flow rate 60 mL/min) to yield 6-methoxy-N-(6-methylpyridazin-3-yl)-1H-benzimidazol-5-amine (200 mg, 0.780 mmol, 29.9% yield) as white solid. LC-MS: m/z=256.1 [M+H]⁺, ESI pos.

### Step 3: 1-[3-acetyl-6-[5-methoxy-6-[(6-methylpyridazin-3-yl)amino]benzimidazol-1-yl]-2-pyridyl]-5-methyl-pyrazole-3-carbonitrile

Error! Objects cannot be created from editing field codes.

Prepared in analogy to example 64, step 2 using 6-methoxy-N-(6-methylpyridazin-3-yl)-1H-benzimidazol-5-amine (200.0 mg, 0.780 mmol, 1.0 equiv.) and 1-(3-acetyl-6-chloro-2-pyridyl)-5-methyl-pyrazole-3-carbonitrile (245.08 mg, 0.940 mmol, 1.2 equiv., prepared in example 64, step 1). Separation of the isomers by preparative HPLC (Welch Ultimate XB-SiOH 250 mm×50 mm×10 μm, gradient 10-50% EtOH (with 0.1% ammonium hydroxide) in heptane over 15 min, then 100% EtOH (with 0.1% ammonium hydroxide) for 3 min) yielded 1-[3-acetyl-6-[5-methoxy-6-[(6-methylpyridazin-3-yl)amino]benzimidazol-1-yl]-2-pyridyl]-5-methyl-pyrazole-3-carbonitrile (120 mg, 0.250 mmol, 31.9% yield) as yellow solid and 1-[3-acetyl-6-[6-methoxy-5-[(6-methylpyridazin-3-yl)amino]benzimidazol-1-yl]-2-pyridyl]-5-methyl-pyrazole-3-carbonitrile (20 mg, 0.040 mmol, 5.3% yield) as yellow solid. Regioisomer 1: LC-MS: m/z=480.1 [M+H]⁺, ESI pos. Regioisomer 2: LC-MS: m/z=480.1 [M+H]⁺, ESI pos. ¹H NMR (400 MHz, CD₃OD) δ=2.25 (s, 3H) 2.58 (d, J=1.38 Hz, 6H) 4.01-4.06 (m, 3H) 6.85 (d, J=0.63 Hz, 1H) 7.29-7.32 (m, 1H) 7.35-7.39 (m, 2H) 8.11-8.17 (m, 1H) 8.52 (d, J=8.38 Hz, 1H) 8.83 (s, 1H) 9.28 (s, 1H).

### Step 4: 1-[3-(-hydroxyethyl)-6-[5-methoxy-6-[(6-methylpyridazin-3-yl)amino]benzimidazol-1-yl]-2-pyridyl]-5-methyl-pyrazole-3-carbonitrile

Error! Objects cannot be created from editing field codes.

Prepared in analogy to example 53, step 4 using 1-[3-acetyl-6-[5-methoxy-6-[(6-methylpyridazin-3-yl)amino]benzimidazol-1-yl]-2-pyridyl]-5-methyl-pyrazole-3-carbonitrile (100.0 mg, 0.210 mmol, 1.0 equiv.) and NaBH₄ (23.67 mg, 0.630 mmol, 3.0 equiv.) in THF/MeOH (4:1) to yield 1-[3-(1-hydroxyethyl)-6-[5-methoxy-6-[(6-methylpyridazin-3-yl)amino]benzimidazol-1-yl]-2-pyridyl]-5-methyl-pyrazole-3-carbonitrile (46.8 mg, 0.100 mmol, 46.6% yield) as white solid by lyophilization. LC-MS: m/z=482.2 [M+H]⁺, ESI pos. ¹H NMR (400 MHz, CD₃OD) δ ppm 1.44 (d, J=6.36 Hz, 3H) 2.40 (s, 3H) 2.59 (s, 3H) 4.02 (s, 3H) 4.74-4.83 (m, 1H) 6.82 (d, J=0.61 Hz, 1H) 7.27-7.32 (m, 1H) 7.34-7.39 (m, 2H) 8.16 (d, J=8.44 Hz, 1H) 8.55-8.60 (m, 1H) 8.75-8.78 (m, 1H) 9.10 (s, 1H).

### Example 143

### 1-[6-[5-[(6-Methylpyridazin-3-yl)amino]benzimidazol-1-yl]-2-[3-methyl-1-(2,2,2-trifluoroethyl)pyrazol-4-yl]-3-pyridyl]ethanol

Error! Objects cannot be created from editing field codes.

### Step 1: 4-bromo-3-methyl-1-(2,2,2-trifluoroethyl)pyrazole and 4-bromo-5-methyl-1-(2,2,2-trifluoroethyl)pyrazole

Error! Objects cannot be created from editing field codes.

A mixture of 4-bromo-3-methylpyrazole (15.0 g, 93.17 mmol, 1.0 equiv.), 2,2,2-trifluoroethyl trifluoromethanesulfonate (22.71 g, 97.83 mmol, 1.05 equiv.) and Cs₂CO₃ (25.33 g, 186.34 mmol, 2 equiv.) in DMF (150 mL) was stirred at 100° C. for 12 hours. The reaction mixture was filtered and the filtrate was diluted with H₂O and extracted with EtOAc. The combined organic layers were washed with brine, dried over Na₂SO₄ and the volatiles removed under reduced pressure. The residue was purified by fish column chromatography (silica gel, 10-16% EtOAc in DCM) to yield a mixture of 4-bromo-5-methyl-1-(2,2,2-trifluoroethyl)pyrazole (6.5 g, 26.75 mmol, 28.7% yield) and 4-bromo-3-methyl-1-(2,2,2-trifluoroethyl)pyrazole (13 g, 53.49 mmol, 57.4% yield) as off-white oil. LC-MS: m/z=242.9 [M+H]⁺, ESI pos.

### Step 2: 3-methyl-4-(4,4,5,5-tetramethyl-1,3,2-dioxaborolan-2-yl)-1-(2,2,2-trifluoroethyl)pyrazole

Error! Objects cannot be created from editing field codes.

To a mixture of bis(pinacolato)diboron (17.24 g, 67.9 mmol, 1.5 equiv.) and 4-bromo-3-methyl-1-(2,2,2-trifluoroethyl)pyrazole (11.0 g, 45.26 mmol, 1.0 equiv.) in 1,4-dioxane (200 mL) was added Pd(dppf)Cl₂ CH₂Cl₂ (3.7 g, 4.53 mmol, 0.10 equiv.) and KOAc (8.9 g, 90.53 mmol, 2.0 equiv.), the mixture was stirred at 100° C. for 16 h under an inert atmosphere. The mixture was concentrated under reduced pressure and purified by flash chromatography (silica gel, 10-20% EtOAc in PE) to yield 3-methyl-4-(4,4,5,5-tetramethyl-1,3,2-dioxaborolan-2-yl)-1-(2,2,2-trifluoroethyl)pyrazole (15.1 g, 52.1 mmol, 84.0% yield) as yellow gum. LC-MS: m/z=291.1 [M+H]⁺, ESI pos.

### Step 4: 1-[6-[5-[(6-methylpyridazin-3-yl)amino]benzimidazol-1-yl]-2-[3-methyl-l-(2,2,2-trifluoroethyl)pyrazol-4-yl]-3-pyridyl]ethanone and 1-[6-[6-[(6-methylpyridazin-3-yl)amino]benzimidazol-1-yl]-2-[5-methyl-1-(2,2,2-trifuoroethyl)pyrazol-4-yl]-3-pyridyl]ethanone

Error! Objects cannot be created from editing field codes.

Prepared in analogy to example 135, step 2 using 1-[2-chloro-6-[5-[(6-methylpyridazin-3-yl)amino]benzimidazol-1-yl]-3-pyridyl]ethanone (0.5 g, 1.32 mmol, 1 equiv., prepared in example 76, step 2) and 3-methyl-4-(4,4,5,5-tetramethyl-1,3,2-dioxaborolan-2-yl)-1-(2,2,2-trifluoroethyl)pyrazole (574.33 mg, 1.98 mmol, 1.5 equiv.). Purification by SFC (Chiralpak AD-3 50 mm×4.6 mm, 3 μm, 40% ⁱPrOH+CH₃CN with 0.05% Et₂NH, flow rate 3 mL/min, back pressure 100 bar)) yielded 1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanone (319 mg, 0.63 mmol, 47.7% yield) as yellow solid and 1-[6-[6-[(6-methylpyridazin-3-yl)amino]benzimidazol-1-yl]-2-[5-methyl-1-(2,2,2-trifluoroethyl)pyrazol-4-yl]-3-pyridyl]ethanone (190 mg, 0.376 mmol, 28.5% yield) as yellow solid. LC-MS: m/z=507.2 [M+H]⁺, ESI pos. ¹H NMR (400 MHz, CD₃OD) δ=8.92 (s, 1H), 8.39-8.17 (m, 3H), 7.97 (s, 1H), 7.87 (d, J=8.4 Hz, 1H), 7.68-7.57 (m, 1H), 7.35 (d, J=9.2 Hz, 1H), 7.13 (d, J=9.2 Hz, 1H), 5.01-4.94 (m, 2H), 2.53 (s, 3H), 2.39 (s, 3H), 2.36 (s, 3H).

### Step 5: 1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanol

Error! Objects cannot be created from editing field codes.

Prepared in analogy to example 53, step 4 using 1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanone (750.0 mg, 1.48 mmol, 1.0 equiv.) and NaBH₄ (281.58 mg, 7.4 mmol, 5 equiv.) at −70° C. to yield 1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanol (450 mg, 0.880 mmol, 69.0% yield) as yellow solid. LC-MS: m/z=509.1 [M+H]⁺, ESI pos.

### Example 144

### 1-[3-(1-Hydroxyethyl)-6-[6-[(6-methylpyridazin-3-yl)amino]benzimidazol-1-yl]-2-pyridyl]-N,5-dimethyl-pyrazole-3-carboxamide

Error! Objects cannot be created from editing field codes.

### Step 1: N,5-dimethyl-1H-pyrazole-3-carboxamide

Error! Objects cannot be created from editing field codes.

To a stirred solution of 3-methyl-1H-pyrazole-5-carboxylic acid ethyl ester (280 mg, 1.82 mmol, 1.0 equiv.) at RT in MeOH (2 mL) under an argon atmosphere was added methylamine, 41% in water (2.75 g, 3.08 mL, 36.32 mmol, 20 equiv). The mixture was heated to 60° C. and stirring at that temperature was continued overnight. The mixture was cooled to RT and concentrated to dryness to yield N,3-dimethyl-1H-pyrazole-5-carboxamide as white solid, which was used in the next step without further purification. LC-MS: m/z=140.1 [M+H]⁺, ESI pos.

### Step 2: 1-(3-acetyl-6-chloro-2-pyridyl)-N,5-dimethyl-pyrazole-3-carboxamide

Error! Objects cannot be created from editing field codes.

Prepared in analogy to example 62, step 1 using N,5-dimethyl-1H-pyrazole-3-carboxamide (used as crude from the previous step) and 1-(6-chloro-2-fluoro-3-pyridyl)ethanone (296.2 mg, 1.71 mmol, 1.0 equiv.) to yield 1-(3-acetyl-6-chloro-2-pyridyl)-N,5-dimethyl-pyrazole-3-carboxamide (142 mg, 24.7% yield) as bowrn solid. LC-MS: m/z=293.1 [M+H]⁺, ESI pos.

### Step 3: 1-[3-acetyl-6-[6-[(6-methylpyridazin-3-yl)amino]benzimidazol-1-yl]-2-pyridyl]-N,5-dimethyl-pyrazole-3-carboxamide and 1-[3-acetyl-6-[5-[(6-methylpyridazin-3-yl)amino]benzimidazol-1-yl]-2-pyridyl]-N,5-dimethyl-pyrazole-3-carboxamide

Error! Objects cannot be created from editing field codes.

Prepared in analogy to example 65, step 1 using 1-(3-acetyl-6-chloro-2-pyridyl)-N,5-dimethyl-pyrazole-3-carboxamide (137 mg, 0.407 mmol, 1 equiv.) and 1H-benzimidazol-5-yl-(6-methylpyridazin-3-yl)amine (101.91 mg, 0.407 mmol, 1 equiv.) over 8 h to yield 1-[3-acetyl-6-[6-[(6-methylpyridazin-3-yl)amino]benzimidazol-1-yl]-2-pyridyl]-N,5-dimethyl-pyrazole-3-carboxamide (40 mg, 19.6% yield) and 1-[3-acetyl-6-[5-[(6-methylpyridazin-3-yl)amino]benzimidazol-1-yl]-2-pyridyl]-N,5-dimethyl-pyrazole-3-carboxamide (33 mg, 15.5% yield) as orange solid after separation of the isomers by flash column chromatography. Regiosisomer 1: LC-MS: m/z=482.2 [M+H]⁺, ESI pos. ¹H NMR (600 MHz, DMSO-d₆) δ ppm 9.28 (s, 1H), 9.11 (s, 1H), 8.46 (d, J=2.2 Hz, 1H), 8.44 (d, J=8.4 Hz, 1H), 8.24 (d, J=8.5 Hz, 1H), 8.17 (d, J=9.1 Hz, 1H), 7.53 (dd, J=8.9, 2.2 Hz, 1H), 7.35 (d, J=9.1 Hz, 1H), 7.11 (d, J=9.1 Hz, 1H), 6.79 (d, J=0.8 Hz, 1H), 2.74 (d, J=4.7 Hz, 3H), 2.55 (d, J=0.8 Hz, 3H), 2.48 (s, 3H), 2.04 (s, 3H) Regioisomer 2: LC-MS: m/z=482.2 [M+H]⁺, ESI pos. ¹H NMR (600 MHz, DMSO-d₆) δ=9.32 (s, 1H), 8.98 (s, 1H), 8.81 (d, J=2.0 Hz, 1H), 8.43-8.49 (m, 1H), 8.18 (d, J=8.5 Hz, 1H), 8.04 (q, J=4.7 Hz, 1H), 7.71 (d, J=8.7 Hz, 1H), 7.53 (dd, J=8.7, 2.1 Hz, 1H), 7.30 (d, J=9.1 Hz, 1H), 7.09 (d, J=9.1 Hz, 1H), 6.73 (d, J=0.9 Hz, 1H), 2.74 (d, J=4.7 Hz, 3H), 2.55 (d, J=0.7 Hz, 3H), 2.48 (s, 3H), 2.04 (s, 3H).

### Step 4: 1-[3-(1-hydroxyethyl)-6-[6-[(6-methylpyridazin-3-yl)amino]benzimidazol-1-yl]-2-pyridyl]-N,5-dimethyl-pyrazole-3-carboxamide

Error! Objects cannot be created from editing field codes.

Prepared in analogy to example 53, step 4 using 1-[3-acetyl-6-[6-[(6-methylpyridazin-3-yl)amino]benzimidazol-1-yl]-2-pyridyl]-N,5-dimethyl-pyrazole-3-carboxamide (37 mg, 0.077 mmol, 1.0 equiv.) in MeOH/THF (1:1) to yield 1-[3-(1-hydroxyethyl)-6-[6-[(6-methylpyridazin-3-yl)amino]benzimidazol-1-yl]-2-pyridyl]-N,5-dimethyl-pyrazole-3-carboxamide (23 mg, 60.7% yield) as light brown solid. LC-MS: m/z=484.3 [M+H]⁺, ESI pos.

### Example 145

### 1-[2-(5-Methyl-3-methylsulfonyl-pyrazol-1-yl)-6-[5-[(6-methylpyridazin-3-yl)amino]benzimidazol-1-yl]-3-pyridyl]ethanol

Error! Objects cannot be created from editing field codes.

### Step 1: 1-[2-(3-bromo-5-methyl-pyrazol-1-yl)-6-chloro-3-pyridyl]ethanone

Error! Objects cannot be created from editing field codes.

Prepared in analogy to example 64, step 1 using 1-(6-chloro-2-fluoro-3-pyridyl)ethanone (CAS #1260663-13-5, 2.0 g, 11.52 mmol, 1.0 equiv.) and 3-bromo-5-methyl-1H-pyrazole (1.86 g, 11.52 mmol, 1.0 equiv.) to yield 1-[2-(3-bromo-5-methyl-pyrazol-1-yl)-6-chloro-3-pyridyl]ethanone (1.8 g, 5.72 mmol, 47.2% yield) as light yellow solid. LC-MS: m/z=313.9 [M+H]⁺ and 315.9 [M+H]⁺, (bromo isotopes) ESI pos. ¹H NMR (400 MHz, CDCl₃) δ=7.91 (d, J=8.1 Hz, 1H), 7.39 (d, J=8.1 Hz, 1H), 6.29 (d, J=0.8 Hz, 1H), 2.60 (d, J=0.8 Hz, 3H), 2.14 (s, 3H).

### Step 2: 1-[2-(3-bromo-5-methyl-pyrazol-1-yl)-6-[5-[(6-methylpyridazin-3-yl)amino]benzimidazol-1-yl]-3-pyridyl]ethanone

Error! Objects cannot be created from editing field codes.

Prepared in analogy to example 64, step 2 using 1-[2-(3-bromo-5-methyl-pyrazol-1-yl)-6-chloro-3-pyridyl]ethanone (1000.0 mg, 3.18 mmol, 1.0 equiv.) and N-(6-methylpyridazin-3-yl)-1H-benzimidazol-5-amine (716.06 mg, 3.18 mmol, 1.0 equiv., prepared in example 64, intermediate 1) to yield 1-[2-(3-bromo-5-methyl-pyrazol-1-yl)-6-[5-[(6-methylpyridazin-3-yl)amino]benzimidazol-1-yl]-3-pyridyl]ethanone (373 mg, 0.740 mmol, 23.31% yield) as a yellow solid. LC-MS: m/z=503.1 [M+H]⁺ and 505.1 [M+H]⁺, ESI pos.

### Step 3: 1-[2-(5-methyl-3-methylsulfonyl-pyrazol-1-yl)-6-[5-[(6-methylpyridazin-3-yl)amino]benzimidazol-1-yl]-3-pyridyl]ethanone

Error! Objects cannot be created from editing field codes.

A mixture of 1-[2-(3-bromo-5-methyl-pyrazol-1-yl)-6-[5-[(6-methylpyridazin-3-yl)amino]benzimidazol-1-yl]-3-pyridyl]ethanone (378.0 mg, 0.750 mmol, 1.0 equiv.), copper (I) triflate (23.95 mg, 0.11 mmol, 0.150 equiv.), (1R,2R)—N,N-dimethyl-1,2-cyclohexanediamine (32.05 mg, 0.230 mmol, 0.30 equiv.) and sodium methanesulphinate (115.0 mg, 1.13 mmol, 1.5 equiv.) in DMSO (12 mL) was stirred at 95° C. for 16 h under inert atmosphere. The mixture was filtered and purified by preparative HPLC (formic acid buffer?) to yield 1-[2-(5-methyl-3-methylsulfonyl-pyrazol-1-yl)-6-[5-[(6-methylpyridazin-3-yl)amino]benzimidazol-1-yl]-3-pyridyl]ethanone (168 mg, 0.330 mmol, 43.6% yield) as a yellow solid. LC-MS: m/z=503.1 [M+H]⁺, ESI pos.

### Step 4: 1-[2-(5-methyl-3-methylsulfonyl-pyrazol-1-yl)-6-[5-[(6-methylpyridazin-3-yl)amino]benzimidazol-1-yl]-3-pyridyl]ethanol

Error! Objects cannot be created from editing field codes.

Prepared in analogy to example 53, step 4 using 1-[2-(5-methyl-3-methylsulfonyl-pyrazol-1-yl)-6-[5-[(6-methylpyridazin-3-yl)amino]benzimidazol-1-yl]-3-pyridyl]ethanone (168.0 mg, 0.330 mmol, 1.0 equiv.) and NaBH₄ (50.0 mg, 1.32 mmol, 3.95 equiv.) in DMF/MeOH (3:1) to yield 1-[2-(5-methyl-3-methylsulfonyl-pyrazol-1-yl)-6-[5-[(6-methylpyridazin-3-yl)amino]benzimidazol-1-yl]-3-pyridyl]ethanol (66.3 mg, 0.130 mmol, 39.3% yield) as a yellow solid. LC-MS: m/z=505.1 [M+H]⁺, ESI pos. ¹H NMR (400 MHz, CD₃OD) δ=8.94 (s, 1H), 8.53 (d, J=8.4 Hz, 1H), 8.27 (s, 1H), 8.23 (br s, 1H), 8.19-8.13 (t, J=8.4 Hz, 2H), 7.63-7.57 (m, 1H), 7.40 (d, J=9.2 Hz, 1H), 7.17 (d, J=8.8 Hz, 1H), 6.91 (s, 1H), 4.81 (q, J=7.6 Hz, 1H), 3.30 (s, 3H), 2.56 (s, 3H), 2.47 (s, 3H), 1.45 (d, J=6.4 Hz, 3H).

### Example 146

### 1-[3-(1-Hydroxyethyl)-6-[5-[(2-keto-1-methyl-pyrimidin-4-yl)amino]benzimidazol-1-yl]-2-pyridyl]-5-methyl-pyrazole-3-carbonitrile

Error! Objects cannot be created from editing field codes.

### Step 1: 1-[3-acetyl-6-[5-[(2-keto-1-methyl-pyrimidin-4-yl)amino]benzimidazol-1-yl]-2-pyridyl]-5-methyl-pyrazole-3-carbonitrile

Error! Objects cannot be created from editing field codes.

Prepared in analogy to example 104, step 3 using 1-[3-acetyl-6-(5-bromobenzimidazol-1-yl)-2-pyridyl]-5-methyl-pyrazole-3-carbonitrile (50 mg, 0.119 mmol, 1.0 equiv., prepared in example 65, step 1) and 4-amino-1-methyl-pyrimidin-2-one (29.7 mg, 0.237 mmol, 2.0 equiv.) to yield the title compound (39.8 mg, 68.4% yield) as yellow solid. LC-MS: m/z=466.3 [M+H]⁺, ESI pos.

### Step 2: 1-[3-(1-hydroxyethyl)-6-[5-[(2-keto-1-methyl-pyrimidin-4-yl)amino]benzimidazol-1-yl]-2-pyridyl]-5-methyl-pyrazole-3-carbonitrile

Error! Objects cannot be created from editing field codes.

Prepared in analogy to example 53, step 4 using 1-[3-acetyl-6-[5-[(2-keto-1-methyl-pyrimidin-4-yl)amino]benzimidazol-1-yl]-2-pyridyl]-5-methyl-pyrazole-3-carbonitrile (39.8 mg, 0.081 mmol, 1 equiv.) in MeOH/THF to yield the title compound (19.9 mg, 49.8% yield) as light yellow solid. LC-MS: m/z=468.3 [M+H]⁺, ESI pos.

### Example 148

### 5-Methyl-1-[6-[5-[(6-methylpyridazin-3-yl)amino]benzimidazol-1-yl]-3-(2,2,2-trifluoro-1-hydroxy-ethyl)-2-pyridyl]pyrazole-3-carbonitrile

Error! Objects cannot be created from editing field codes.

### Step 1: 1-(6-chloro-2-fluoro-3-pyridyl)-2,2,2-trifluoro-ethanone

Error! Objects cannot be created from editing field codes.

LDA (4.56 mL, 9.12 mmol, 1.2 equiv.) was added dropwise to a solution of 2-chloro-6-fluoropyridine (1.0 g, 7.6 mmol, 1 equiv.) in THF (20 mL) at −70° C. A yellow suspension was formed. The mixture was stirred at −70° C. for 1 hour, then N-methoxy-N-methyltrifluoroacetamide (1.26 g, 7.99 mmol, 1.05 equiv.) was added dropwise. After addition, the clear yellow solution was stirred at −70° C. for 1 hour. The mixture was quenched with 100 mL sat. aq. NH₄Cl, extracted with EtOAc and the organic layers were concentrated under reduced pressure. The residue was purified by flash column choromatography (silica gel, 25% EtOAc in PE) to yield 1-(6-chloro-2-fluoro-3-pyridyl)-2,2,2-trifluoro-ethanone (600 mg, 2.64 mmol, 34.7% yield) as light brown oil. LC-MS: m/z=246.1 [M+H₂O+H]⁺, ESI pos.

### Step 2: 1-[6-chloro-3-(2,2,2-trifluoroacetyl)-2-pyridyl]-5-methyl-pyrazole-3-carbonitrile

Error! Objects cannot be created from editing field codes.

To mixture of 1-(6-chloro-2-fluoro-3-pyridyl)-2,2,2-trifluoro-ethanone (6.2 g, 27.25 mmol, 1.0 equiv.) and 5-methyl-1H-pyrazole-3-carbonitrile (2.91 g, 27.21 mmol, 1.0 equiv.) in DMSO (50 mL) was added DIPEA (7.9 mL, 54.5 mmol, 2.0 equiv.) dropwise at 0° C. After addition, the mixture was stirred at 20° C. for 3 hours. The mixture was quenched with 100 mL water, extracted with 100 mL EtOAc, and the organic layer concentrated under reduced pressure. The residue was purified by reversed phase preparative HPLC (Waters Xbridge BEH C18 150 mm×50 mm×10 μm, gradient 30-50% CH₃CN in H₂O (with 10 mM NH₄HCO₃) over 22 min, then 100% CH₃CN (5 min), flow rate 140 mL/min) to give 1-[6-chloro-3-(2,2,2-trifluoroacetyl)-2-pyridyl]-5-methyl-pyrazole-3-carbonitrile (3.2 g, 10.17 mmol, 37.3% yield) as a pink solid. LC-MS: m/z=315.1 [M+H]⁺, 333.1 [M+H₂O+H]⁺ ESI pos.

### Step 3: 5-methyl-1-[6-[5-[(6-methylpyridazin-3-yl)amino]benzimidazol-1-yl]-3-(2,2,2-trifluoroacetyl)-2-pyridyl]pyrazole-3-carbonitrile; formic acid and 5-methyl-1-[6-[6-[(6-methylpyridazin-3-yl)amino]benzimidazol-1-yl]-3-(2,2,2-trifluoroacetyl)-2-pyridyl]pyrazole-3-carbonitrile; formic acid

Error! Objects cannot be created from editing field codes.

A mixture of 1-[6-chloro-3-(2,2,2-trifluoroacetyl)-2-pyridyl]-5-methyl-pyrazole-3-carbonitrile (0.4 g, 1.27 mmol, 1.0 equiv.), N-(6-methylpyridazin-3-yl)-1H-benzimidazol-5-amine (301.0 mg, 1.34 mmol, 1.05 equiv.) and DIPEA (0.45 mL, 2.54 mmol, 2.0 equiv.) in DMF (10 mL) was stirred at 100° C. for 16 hours. The mixture was purified by preparative HPLC (Shim-pack C18 150 mm×25 mm×10 μm, gradient 1-30% CH₃CN in H₂O (with 0.225% formic acid) over 10 min, then 100% CH₃CN (2 min), flow rate 25 mL/min, 1 injection) to yield 5-methyl-1-[6-[5-[(6-methylpyridazin-3-yl)amino]benzimidazol-1-yl]-3-(2,2,2-trifluoroacetyl)-2-pyridyl]pyrazole-3-carbonitrile; formic acid (170 mg, 26.6% yield) as dark brown solid. LC-MS: m/z=504.1 [M+H]⁺, ESI pos. and 5-methyl-1-[6-[6-[(6-methylpyridazin-3-yl)amino]benzimidazol-1-yl]-3-(2,2,2-trifluoroacetyl)-2-pyridyl]pyrazole-3-carbonitrile; formic acid (120 mg, 18.8% yield) as dark brown solid. LC-MS: m/z=504.1 [M+H]⁺, ESI pos.

### Step 4: 5-methyl-1-[6-[5-[(6-methylpyridazin-3-yl)amino]benzimidazol-1-yl]-3-(2,2,2-trifluoro-1-hydroxy-ethyl)-2-pyridyl]pyrazole-3-carbonitrile and 5-methyl-1-[6-[6-[(6-methylpyridazin-3-yl)amino]benzimidazol-1-yl]-3-(2,2,2-trifluoro-1-hydroxy-ethyl)-2-pyridyl]pyrazole-3-carbonitrile

Error! Objects cannot be created from editing field codes.

Prepared in analogy to example 53, step 3, using 5-methyl-1-[6-[5-[(6-methylpyridazin-3-yl)amino]benzimidazol-1-yl]-3-(2,2,2-trifluoroacetyl)-2-pyridyl]pyrazole-3-carbonitrile (40.0 mg, 0.080 mmol, 1 equiv.) and NaBH₄ (8.0 mg, 0.210 mmol, 2.7 equiv.) to yield 5-methyl-1-[6-[5-[(6-methylpyridazin-3-yl)amino]benzimidazol-1-yl]-3-(2,2,2-trifluoro-1-hydroxy-ethyl)-2-pyridyl]pyrazole-3-carbonitrile (5.3 mg, 0.010 mmol, 11.9% yield) and 5-methyl-1-[6-[6-[(6-methylpyridazin-3-yl)amino]benzimidazol-1-yl]-3-(2,2,2-trifluoro-1-hydroxy-ethyl)-2-pyridyl]pyrazole-3-carbonitrile (1.0 mg, 0.002 mmol, 2.5% yield) as a white solids after purification by preparative HPLC (Waters Xbridge BEH C18 150 mm×50 mm×5 μm, gradient 29-59% CH₃CN in H₂O (with 10 mM NH₄HCO₃) over 8 min, then 100% CH₃CN (2 min), flow rate 25 mL/min) and separation of the two isomers by preparative NPLC (Welch Ultimate XB-SiOH 250 mm×50 mm×10 μm, gradient 10-50% EtOH (with 0.1% ammonium hydroxide) in heptane over 15 min, then 100% EtOH (with 0.1% ammonium hydroxide) for 3 min), flow rate 100 mL/min). Regioisomer 1: LC-MS: m/z=506.2 [M+H]⁺, ESI pos. ¹H NMR (400 MHz, CDCl₃) δ=8.54-8.47 (m, 1H), 8.42-8.35 (m, 1H), 7.96-7.89 (m, 1H), 7.75-7.68 (m, 1H), 7.66-7.38 (m, 1H), 7.22-7.17 (m, 1H), 7.16-7.12 (m, 1H), 7.10-7.05 (m, 1H), 6.98-6.92 (m, 1H), 5.23-5.14 (m, 1H), 4.13-3.86 (m, 1H), 3.78-3.56 (m, 2H), 2.64-2.57 (m, 3H), 2.49-2.41 (m, 1H), 2.05-1.99 (m, 1H), 1.61-1.53 (m, 3H), 1.52-1.47 (m, 3H). Regioisomer 2: LC-MS: m/z=506.2 [M+H]⁺, ESI pos. ¹H NMR (400 MHz, CD₃OD) δ=8.97-8.95 (m, 1H), 8.64-8.61 (m, 1H), 8.27-8.25 (m, 1H), 8.25-8.22 (m, 1H), 8.18-8.14 (m, 1H), 7.66-7.62 (m, 1H), 7.39-7.36 (m, 1H), 7.16-7.13 (m, 1H), 6.91 (s, 1H), 5.55-5.49 (m, 1H), 2.56-2.54 (m, 3H), 2.45-2.43 (m, 3H), 2.06-2.05 (m, 1H).

### Example 149

### 1-[2-[1-(Difluoromethyl)-3-methyl-pyrazol-4-yl]-6-[5-[(6-methylpyridazin-3-yl)amino]benzimidazol-1-yl]-3-pyridyl]ethanol

Error! Objects cannot be created from editing field codes.

### Step 1: 4-bromo-1-(difluoromethyl)-3-methyl-pyrazole

Error! Objects cannot be created from editing field codes.

To a solution of 4-bromo-3-methylpyrazole (200.0 mg, 1.24 mmol, 1.0 equiv.) in DMF (5 mL) was added sodium chlorodifluoroacetate (378.8 mg, 2.48 mmol, 2.0 equiv.) and potassium carbonate (515.1 mg, 3.73 mmol, 3.0 equiv.). The mixture was heated to 100° C. for 12 hours. The reaction mixture was then diluted with 20 mL water and extracted with EtOAc. The combined extracts were washed with brine and concentrated under reduced pressure. The residue was purified by flash column chromatography (0-20% EtOAc in PE) to yield 4-bromo-1-(difluoromethyl)-3-methyl-pyrazole (200 mg, 0.950 mmol, 66.4% yield) as a colorless oil. LC-MS: m/z=213.0 [M+H]⁺, ESI pos.

### Step 2: 1-(difluoromethyl)-3-methyl-4-(4,4,5,5-tetramethyl-1,3,2-dioxaborolan-2-yl)pyrazole

Error! Objects cannot be created from editing field codes.

Prepared in analogy to example 143, step 2 using bis(pinacolato)diboron (240.7 mg, 0.950 mmol, 2.0 equiv.) and 4-bromo-1-(difluoromethyl)-3-methyl-pyrazole (100 mg, 0.474 mmol, 1.0 equiv.) to yield 1-(difluoromethyl)-3-methyl-4-(4,4,5,5-tetramethyl-1,3,2-dioxaborolan-2-yl)pyrazole (113 mg, 0.436 mmol, 98.4% yield) as a white crystalline solid. LC-MS: m/z=259.2 [M+H]⁺, ESI pos.

### Step 3: 1-[2-[1-(difluoromethyl)-3-methyl-pyrazol-4-yl]-6-[5-[(6-methylpyridazin-3-yl)amino]benzimidazol-1-yl]-3-pyridyl]ethanone

Error! Objects cannot be created from editing field codes.

Prepared in analogy to example 135 Step 2, using of 1-[2-chloro-6-[5-[(6-methylpyridazin-3-yl)amino]benzimidazol-1-yl]-3-pyridyl]ethanone (5.0 mg, 0.010 mmol, 1.0 equiv., prepared in example 76, step 2) and 1-[2-chloro-6-[5-[(6-methylpyridazin-3-yl)amino]benzimidazol-1-yl]-3-pyridyl]ethanone (5.0 mg, 0.010 mmol, 1 equiv.) while running the reaction for 2 h at 100° C. to yield 1-[2-[1-(difluoromethyl)-5-methyl-pyrazol-4-yl]-6-[5-[(6-methylpyridazin-3-yl)amino]benzimidazol-1-yl]-3-pyridyl]ethanone (12 mg, 0.030 mmol, 7.44% yield). LC-MS: m/z 475.2 [M+H]⁺, ESI pos.

### Step 4: 1-[2-[1-(difluoromethyl)-3-methyl-pyrazol-4-yl]-6-[5-[(6-methylpyridazin-3-yl)amino]benzimidazol-1-yl]-3-pyridyl]ethanol

Error! Objects cannot be created from editing field codes.

Prepared in analogy to example 53, Step 3, using 1-[2-[1-(difluoromethyl)-3-methyl-pyrazol-4-yl]-6-[5-[(6-methylpyridazin-3-yl)amino]benzimidazol-1-yl]-3-pyridyl]ethanone (20.0 mg, 0.040 mmol, 1.0 equiv.) and NaBH₄ (4.78 mg, 0.130 mmol, 3 equiv.) to yield 1-[2-[1-(difluoromethyl)-3-methyl-pyrazol-4-yl]-6-[5-[(6-methylpyridazin-3-yl)amino]benzimidazol-1-yl]-3-pyridyl]ethanol (5.9 mg, 0.010 mmol, 29.4% yield) as a white solid. LC-MS: m/z=477.1 [M+H]⁺, ESI pos.

### Example 150

### 1-[3-(1-Hydroxyethyl)-6-[6-[(6-methylpyridazin-3-yl)amino]benzimidazol-1-yl]-2-pyridyl]-5-methyl-pyrazole-4-carbonitrile

Error! Objects cannot be created from editing field codes.

### Step 1: 1-(3-acetyl-6-chloro-2-pyridyl)-3-methyl-pyrazole-4-carbonitrile

Error! Objects cannot be created from editing field codes.

Prepared in analogy to example 62, step 1 using 5-methyl-1H-pyrazole-4-carbonitrile (616.9 mg, 5.76 mmol, 1.0 equiv.) and 1-(6-chloro-2-fluoro-3-pyridyl)ethanone (CAS #1260663-13-5, 1.0 g, 5.76 mmol, 1.0 equiv.) to yield 1-(3-acetyl-6-chloro-2-pyridyl)-3-methyl-pyrazole-4-carbonitrile (900 mg, 3.45 mmol, 59.9% yield) as white solid. LC-MS: m/z=260.9 [M+H]⁺, ESI pos.

### Step 2: 1-[3-acetyl-6-[6-[(6-methylpyridazin-3-yl)amino]benzimidazol-1-yl]-2-pyridyl]-5-methyl-pyrazole-4-carbonitrile and 1-[3-acetyl-6-[5-[(6-methylpyridazin-3-yl)amino]benzimidazol-1-yl]-2-pyridyl]-5-methyl-pyrazole-4-carbonitrile

Error! Objects cannot be created from editing field codes.

Prepared in analogy to example 53, step 3 using 1-(3-acetyl-6-chloro-2-pyridyl)-5-methyl-pyrazole-4-carbonitrile (182.0 mg, 0.700 mmol, 1.0 equiv.), N-(6-methylpyridazin-3-yl)-1H-benzimidazol-5-amine (188.72 mg, 0.840 mmol, 1.2 equiv., prepared in example 64, intermediate 1) and DIPEA (0.24 mL, 1.4 mmol, 2 equiv.) to yield 1-[3-acetyl-6-[5-[(6-methylpyridazin-3-yl)amino]benzimidazol-1-yl]-2-pyridyl]-5-methyl-pyrazole-4-carbonitrile (100 mg, 0.220 mmol, 31.87% yield) as light brown solid and 1-[3-acetyl-6-[6-[(6-methylpyridazin-3-yl)amino]benzimidazol-1-yl]-2-pyridyl]-5-methyl-pyrazole-4-carbonitrile (60 mg, 0.130 mmol, 19.12% yield) as light brown solid after separation by preparative NPLC (preparative NPLC (Welch Ultimate XB-SiOH 250 mm×70 mm×10 μm, gradient 20-60% EtOH in hexane over 20 min, then 100% EtOH (3 min), flow rate 140 mL/min). Regiosisomer 1: LC-MS: m/z=450.2 [M+H]⁺, ESI pos. ¹H NMR (400 MHz, CDCl₃) δ=8.62 (s, 1H), 8.32-8.25 (m, 1H), 8.03-7.97 (m, 1H), 7.94-7.91 (m, 1H), 7.87-7.82 (m, 1H), 7.78-7.72 (m, 1H), 7.50-7.44 (m, 1H), 7.21-7.16 (m, 1H), 7.15-7.09 (m, 1H), 2.79 (s, 3H), 2.62 (s, 3H), 2.24 (s, 3H). Regiosisomer 2: LC-MS: m/z=450.2 [M+H]⁺, ESI pos. ¹H NMR (400 MHz, CDCl₃) δ=8.71-8.64 (m, 1H), 8.58-8.53 (m, 1H), 8.35-8.23 (m, 1H), 7.93-7.89 (m, 1H), 7.86-7.77 (m, 2H), 7.25-7.15 (m, 2H), 7.05-6.94 (m, 2H), 2.80 (s, 3H), 2.64 (s, 3H), 2.28-2.22 (m, 3H).

### Step 3: 1-[3-(-hydroxyethyl)-6-[6-[(6-methylpyridazin-3-yl)amino]benzimidazol-1-yl]-2-pyridyl]-5-methyl-pyrazole-4-carbonitrile

Error! Objects cannot be created from editing field codes.

Prepared in analogy to example 53, Step 3, using of 1-[3-acetyl-6-[6-[(6-methylpyridazin-3-yl)amino]benzimidazol-1-yl]-2-pyridyl]-5-methyl-pyrazole-4-carbonitrile (60.0 mg, 0.130 mmol, 1.0 equiv.) to yield 1-[3-(1-hydroxyethyl)-6-[6-[(6-methylpyridazin-3-yl)amino]benzimidazol-1-yl]-2-pyridyl]-5-methyl-pyrazole-4-carbonitrile (34.5 mg, 0.080 mmol, 57.24% yield) as an off-white solid after preparative HPLC (Phenomenex Synergi C18 150×25 mm×10 μm, gradient 2-32% CH₃CN in H₂O (with 0.225% formic acid) over 10 min, then 100% CH₃CN (2 min), flow rate 25 mL/min, 1 injection). LC-MS: m/z=451.9 [M+H]⁺, ESI pos. ¹H NMR (400 MHz, CD₃OD) δ=8.87-8.74 (m, 2H), 8.56-8.47 (m, 1H), 8.16-8.02 (m, 2H), 7.70-7.61 (m, 1H), 7.41-7.29 (m, 2H), 7.15-7.06 (m, 1H), 4.83-4.80 (m, 1H), 2.58-2.54 (m, 3H), 2.53-2.48 (m, 3H), 1.45-1.36 (m, 3H)

### Example 151

### 1-[3-(1-Hydroxyethyl)-6-[5-[(6-methylpyridazin-3-yl)amino]benzimidazol-1-yl]-2-pyridyl]-5-methyl-pyrazole-4-carbonitrile

Error! Objects cannot be created from editing field codes.

Prepared in analogy to example 53, Step 3 using 1-[3-acetyl-6-[5-[(6-methylpyridazin-3-yl)amino]benzimidazol-1-yl]-2-pyridyl]-5-methyl-pyrazole-4-carbonitrile (100.0 mg, 0.220 mmol, 1.0 equiv., prepared in example 150, step 2) in CHCl₃/MeOH (1:1) to yield 1-[3-(1-hydroxyethyl)-6-[5-[(6-methylpyridazin-3-yl)amino]benzimidazol-1-yl]-2-pyridyl]-5-methyl-pyrazole-4-carbonitrile (58.5 mg, 0.130 mmol, 58.24% yield) as yellow solid after preparative HPLC (Phenomenex Synergi C18 150×25 mm×10 μm, gradient 3-36% CH₃CN in H₂O (with 0.225% formic acid) over 11 min, then 100% CH₃CN (2 min), flow rate 25 mL/min). LC-MS: m/z=452.0 [M+H]⁺, ESI pos. ¹H NMR (400 MHz, CD₃OD) δ=8.87 (s, 1H), 8.51-8.44 (m, 1H), 8.24-8.18 (m, 1H), 8.13-8.04 (m, 3H), 7.59-7.54 (m, 1H), 7.40-7.34 (m, 1H), 7.17-7.10 (m, 1H), 4.82-4.80 (m, 1H), 2.53 (s, 6H), 1.41 (d, J=6.5 Hz, 3H).

### Example 152

### 1-[3-(1-Hydroxyethyl)-6-[5-[(6-methylpyridazin-3-yl)amino]benzimidazol-1-yl]-2-pyridyl]-N,5-dimethyl-pyrazole-3-carboxamide

Error! Objects cannot be created from editing field codes.

Prepared in analogy to example 55, Step 4, using of 1-[3-acetyl-6-[5-[(6-methylpyridazin-3-yl)amino]benzimidazol-1-yl]-2-pyridyl]-N,5-dimethyl-pyrazole-3-carboxamide (30 mg, 0.062 mmol, 1.0 equiv., prepared in example 144, step 3) in MeOH/THF (1:1) while stirring overnight to yield to yield the title compound (14 mg, 46% yield) as a light brown solid. LC-MS: m/z=484.3 [M+H]⁺, ESI pos.

### Example 155

### 1-[3-(1-Hydroxyethyl)-6-[5-[(2-keto-1-methyl-4-piperidyl)amino]benzimidazol-1-yl]-2-pyridyl]-5-methyl-pyrazole-3-carbonitrile

Error! Objects cannot be created from editing field codes.

### Step 1: 1-[3-acetyl-6-[5-[(2-keto-1-methyl-4-piperidyl)amino]benzimidazol-1-yl]-2-pyridyl]-5-methyl-pyrazole-3-carbonitrile

Error! Objects cannot be created from editing field codes.

Prepared in analogy to example 104, Step 2 with 4-amino-1-methyl-2-piperidone; dihydrochloride (23.87 mg, 0.119 mmol, 2.0 equiv.) for 6 h to afford the title compound (10.7 mg, 36.6% yield) as yellow solid. LC-MS: m/z=469.3 [M+H]⁺, ESI pos.

### Step 2: 1-[3-(1-hydroxyethyl)-6-[5-[(2-keto-1-methyl-4-piperidyl)amino]benzimidazol-1-yl]-2-pyridyl]-5-methyl-pyrazole-3-carbonitrile

Error! Objects cannot be created from editing field codes.

Prepared in analogy to example 53, step 4 in MeOH/THF (1:1) to afford the title compound (6.5 mg, 58.7% yield) as yellow solid. LC-MS: m/z=471.3 [M+H]⁺, ESI pos.

### Example 158

### [2-(3-methoxy-5-methyl-pyrazol-1-yl)-6-[5-[(6-methylpyridazin-3-yl)amino]benzimidazol-1-yl]-3-pyridyl]methanol

Error! Objects cannot be created from editing field codes.

### Intermediate 1: N-(6-methylpyridazin-3-yl)-1H-benzimidazol-5-amine

Error! Objects cannot be created from editing field codes.

A mixture of 5-aminobenzimidazole (8.0 g, 60.1 mmol, 1.0 equiv.) and 3-chloro-6-methylpyridazine (7.34 g, 57.08 mmol, 0.950 equiv.) in ⁱPrOH (120 mL) was stirred at 120° C. for 72 hours. The dark brown suspension was concentrated in vacuo and the residue was triturated in MeOH (60 mL). The solid was collected by filtration and it was triturated in DCM (40 mL). The product was collected by filtration, washed with DCM and dried. The title compound (10 g, 71.3% yield) was obtained as brown solid. LC-MS: m/z=226.0 [M+H]⁺, ESI pos. ¹H NMR (400 MHz, DMSO-d6): δ=9.60 (br s, 1H), 8.72 (s, 1H), 8.52 (d, J=1.7 Hz, 1H), 7.63 (d, J=8.8 Hz, 1H), 7.44 (dd, J=2.0, 8.8 Hz, 1H), 7.39 (d, J=9.2 Hz, 1H), 7.21 (d, J=9.2 Hz, 1H), 5.04-4.15 (m, 1H), 2.49 (s, 3H).

### Step 1: methyl 6-chloro-2-(3-methoxy-5-methyl-pyrazol-1-yl)pyridine-3-carboxylate

Error! Objects cannot be created from editing field codes.

Prepared in analogy to example 133, Step 1 using methyl 6-chloro-2-fluoro-pyridine-3-carboxylate (1.0 g, 5.28 mmol, 1.0 equiv.) and and 3-methoxy-5-methyl-1H-pyrazole (600.0 mg, 5.35 mmol, 1.0 equiv.) to give methyl 6-chloro-2-(3-methoxy-5-methyl-pyrazol-1-yl)pyridine-3-carboxylate (1.4 g, 4.97 mmol, 94.2% yield) as white solid. LC-MS: m/z=282.2 [M+H]⁺, ESI pos.

### Step 2: methyl 2-(3-methoxy-5-methyl-pyrazol-1-yl)-6-[5-[(6-methylpyridazin-3-yl)amino]benzimidazol-1-yl]pyridine-3-carboxylate

Error! Objects cannot be created from editing field codes.

A mixture of N-(6-methylpyridazin-3-yl)-1H-benzimidazol-5-amine (0.92 g, 4.07 mmol, 1.04 equiv., intermediate 1), methyl 6-chloro-2-(3-methoxy-5-methyl-pyrazol-1-yl)pyridine-3-carboxylate (1.1 g, 3.91 mmol, 1.0 equiv.) and K₂CO₃ (1.65 g, 11.94 mmol, 3.1 equiv.) in DMSO (50 mL) was stirred at 50° C. for 12 hours. The mixture was cooled to RT and poured into H₂O (500 mL). A solid precipitated out. This was extracted with EtOAc (3×400 mL). The combined organic layers were concentrated. The residue was purified by preparative HPLC: column Phenomenex Luna C18 (250 mm×70 mm×15 μm. Flow rate 140 mL/min. Gradient: 20% to 50% CH₃CN in (H₂O with 0.225% formic acid v/v) (35 min) then 100% CH₃CN (1 min). A mixture of methyl 2-(3-methoxy-5-methyl-pyrazol-1-yl)-6-[6-[(6-methylpyridazin-3-yl)amino]benzimidazol-1-yl]pyridine-3-carboxylate (370 mg, 0.79 mmol, 20.1% yield) and methyl 2-(3-methoxy-5-methyl-pyrazol-1-yl)-6-[5-[(6-methylpyridazin-3-yl)amino]benzimidazol-1-yl]pyridine-3-carboxylate (350 mg, 0.74 mmol, 18.1% yield) as light yellow solids was obtained. LC-MS: m/z=471.1 [M+H]⁺, ESI pos. This mixture was purified by preparative NPLC: column Welch Ultimate XB-SiOH (250 mm×70 mm×10 um). Flow rate 140 mL/min. Gradient: 20% to 60% EtOH in hexane (20 min) then 100% EtOH (3 min).

### Step 3: [2-(3-methoxy-5-methyl-pyrazol-1-yl)-6-[5-[(6-methylpyridazin-3-yl)amino]benzimidazol-1-yl]-3-pyridyl]methanol

Error! Objects cannot be created from editing field codes.

A solution of methyl 2-(3-methoxy-5-methyl-pyrazol-1-yl)-6-[5-[(6-methylpyridazin-3-yl)amino]benzimidazol-1-yl]pyridine-3-carboxylate (30.0 mg, 0.060 mmol, 1.0 equiv.) in DCM (4 mL) was added DIBAL-H (0.14 mL, 0.140 mmol, 2.2 equiv.) at −76° C. over 5 min. The mixture was stirred at −76° C. for 1 hour. Another portion of DIBAL-H (0.14 mL, 0.140 mmol, 2.2 equiv.) was added after 2 hours and 4 hours at −76° C., and the reaction mixture stirred for another hour at this temperature. Then the reaction mixture was diluted with 5 mL THF and warmed to 0° C., the reaction mixture was quenched by addition of 0.16 mL of H₂O, followed by 0.16 mL of 15% aq. NaOH and 0.4 mL H₂O in this order. After being stirred at RT for 15 min, the MgSO₄ was added to the reaction mixture which was stirred at RT for 15 min. After filtration, the volatiles were removed under reduced pressure. The residue was purified by preparative TLC (silica gel, 9% MeOH in DCM) to give [2-(3-methoxy-5-methyl-pyrazol-1-yl)-6-[5-[(6-methylpyridazin-3-yl)amino]benzimidazol-1-yl]-3-pyridyl]methanol (20 mg, 0.050 mmol, 68.8% yield) as light yellow solid. LC-MS: m/z=443.1 [M+H]⁺, ESI pos. ¹H NMR (400 MHz, CD₃OD): δ=8.84 (s, 1H), 8.34 (d, J=8.3 Hz, 1H), 8.21 (d, J=2.0 Hz, 1H), 8.13 (d, J=8.9 Hz, 1H), 7.92 (d, J=8.3 Hz, 1H), 7.61 (dd, J=2.1, 8.9 Hz, 1H), 7.35 (d, J=9.1 Hz, 1H), 7.13 (d, J=9.1 Hz, 1H), 5.85 (s, 1H), 4.69 (s, 2H), 3.90 (s, 3H), 2.53 (s, 3H), 2.41 (s, 3H).

### Example 159

### 1-[2-(3,5-Dimethylisoxazol-4-yl)-6-[5-[(6-methylpyridazin-3-yl)amino]benzimidazol-1-yl]-3-pyridyl]ethanol

Error! Objects cannot be created from editing field codes.

### Step 1: 1-[2-(3,5-dimethylisoxazol-4-yl)-6-[5-[(6-methylpyridazin-3-yl)amino]benzimidazol-1-yl]-3-pyridyl]ethanone

Error! Objects cannot be created from editing field codes.

Prepared in analogy to example 143, step 2 using (3,5-dimethylisoxazol-4-yl)boronic acid (26.0 mg, 0.180 mmol, 1.4 equiv.) and of 1-[2-chloro-6-[5-[(6-methylpyridazin-3-yl)amino]benzimidazol-1-yl]-3-pyridyl]ethanone (50.0 mg, 0.130 mmol, 1.0 equiv., prepared in example 76, step 2) to give 1-[2-(3,5-dimethylisoxazol-4-yl)-6-[5-[(6-methylpyridazin-3-yl)amino]benzimidazol-1-yl]-3-pyridyl]ethanone (25 mg, 0.060 mmol, 43.1% yield) as yellow solid. LC-MS: 440.2 [M+H]⁺, ESI pos.

### Step 2: 1-[2-(3,5-dimethylisoxazol-4-yl)-6-[5-[(6-methylpyridazin-3-yl)amino]benzimidazol-1-yl]-3-pyridyl]ethanol

Error! Objects cannot be created from editing field codes.

Prepared in analogy to example 53, step 4 to yield 1-[2-(3,5-dimethylisoxazol-4-yl)-6-[5-[(6-methylpyridazin-3-yl)amino]benzimidazol-1-yl]-3-pyridyl]ethanol (9.8 mg, 0.020 mmol, 39.0% yield) as white solid. Lc-MS: m/z=442.2 [M+H]⁺, ESI pos.

### Example 160

### 1-[3-(1-Hydroxyethyl)-6-[5-[(4-methoxypyridazin-3-yl)amino]benzimidazol-1-yl]-2-pyridyl]-5-methyl-pyrazole-3-carbonitrile

Error! Objects cannot be created from editing field codes.

### Step 1: 1-[3-acetyl-6-[5-[(4-methoxypyridazin-3-yl)amino]benzimidazol-1-yl]-2-pyridyl]-5-methyl-pyrazole-3-carbonitrile

Error! Objects cannot be created from editing field codes.

Was prepared in analogy to example 104, step 2 using (4-methoxypyridazin-3-yl)amine (23.76 mg, 0.190 mmol, 2.0 equiv.) to yield the title compound (29.6 mg, 63.6% yield) as yellow solid. LC-MS: 466.3 [M+H]⁺, ESI pos.

### Step 2: 1-[3-(-hydroxyethyl)-6-[5-[(4-methoxypyridazin-3-yl)amino]benzimidazol-1-yl]-2-pyridyl]-5-methyl-pyrazole-3-carbonitrile

Error! Objects cannot be created from editing field codes.

In analogy to example 53, step 4, in MeOH/THF (1:1) the title compound (18.9 mg, 63.6% yield) was obtained as light yellow solid. LC-MS: 468.3 [M+H]⁺, ESI pos.

### Example 161

### 1-[3-(1-Hydroxyethyl)-6-[5-[(5-keto-1-methyl-pyrrolidin-3-yl)amino]benzimidazol-1-yl]-2-pyridyl]-5-methyl-pyrazole-3-carbonitrile

Error! Objects cannot be created from editing field codes.

### Step 1: 1-[3-acetyl-6-[5-[(5-keto-1-methyl-pyrrolidin-3-yl)amino]benzimidazol-1-yl]-2-pyridyl]-5-methyl-pyrazole-3-carbonitrile

Error! Objects cannot be created from editing field codes.

Was prepared in analogy to example 104, step 2 using 4-amino-1-methyl-2-pyrrolidone (27.1 mg, 0.237 mmol, 2 equiv.) to yield the title compound (43.1 mg, 75.9% yield). LC-MS: 455.3 [M+H]⁺, ESI pos.

### Step 2: 1-[3-(1-hydroxyethyl)-6-[5-[(5-keto-1-methyl-pyrrolidin-3-yl)amino]benzimidazol-1-yl]-2-pyridyl]-5-methyl-pyrazole-3-carbonitrile

Error! Objects cannot be created from editing field codes.

In analogy to example 53, step 4, in MeOH/THF (1:1) the title compound (36.7 mg, 81.4% yield) was obtained as yellow solid. LC-MS: 457.3 [M+H]⁺, ESI pos.

### Example 162

### 1-[6-[5-[(6-Methylpyridazin-3-yl)amino]benzimidazol-1-yl]-2-(3-methyl-4-pyridyl)-3-pyridyl]ethanol

Error! Objects cannot be created from editing field codes.

### Step 1: 1-[6-[5-[(6-methylpyridazin-3-yl)amino]benzimidazol-1-yl]-2-(3-methyl-4-pyridyl)-3-pyridyl]ethanone

Error! Objects cannot be created from editing field codes.

Prepared in analogy to example 143, step 2 using 3-methylpyridine-4-boronic acid (36.15 mg, 0.260 mmol, 2 equiv.) and of 1-[2-chloro-6-[5-[(6-methylpyridazin-3-yl)amino]benzimidazol-1-yl]-3-pyridyl]ethanone (50.0 mg, 0.130 mmol, 1.0 equiv., prepared in example 76, step 2) to give 1-[6-[5-[(6-methylpyridazin-3-yl)amino]benzimidazol-1-yl]-2-(3-methyl-4-pyridyl)-3-pyridyl]ethanone (22 mg, 0.050 mmol, 38.28% yield) as a yellow solid. LC-MS: m/z=436.2, [M+H]⁺, ESI pos.

### Step 2: 1-[6-[5-[(6-methylpyridazin-3-yl)amino]benzimidazol-1-yl]-2-(3-methyl-4-pyridyl)-3-pyridyl]ethanol

Error! Objects cannot be created from editing field codes.

In analogy to example 53, step 4, using 5.0 equivalents of NaBH₄, 1-[6-[5-[(6-methylpyridazin-3-yl)amino]benzimidazol-1-yl]-2-(3-methyl-4-pyridyl)-3-pyridyl]ethanol (8.9 mg, 0.020 mmol, 44.3% yield) was obtained as a white solid. LC-MS: m/z=438.2, [M+H]⁺, ESI pos.

### Example 163

### 1-[2-[4-(Cyclopropylamino)pyrimidin-5-yl]-6-[5-[(6-methylpyridazin-3-yl)amino]benzimidazol-1-yl]-3-pyridyl]ethanol

Error! Objects cannot be created from editing field codes.

### Step 1: [4-(cyclopropylamino)pyrimidin-5-yl]boronic acid

Error! Objects cannot be created from editing field codes.

Prepared in analogy to example 143, step 2 using 5-bromo-N-cyclopropyl-pyrimidin-4-amine (1.0 g, 4.67 mmol, 1.0 equiv.) and bis(pinacolato)diboron (1779.45 mg, 7.01 mmol, 1.5 equiv.) to yield [4-(cyclopropylamino)pyrimidin-5-yl]boronic acid (368 mg, 2.06 mmol, 44.0% yield) as white solid. LC-MS: m/z=180.1, [M+H]⁺, ESI pos.

### Step 2: 1-[2-chloro-6-[5-[(6-methylpyridazin-3-yl)amino]benzimidazol-1-yl]-3-pyridyl]ethanol

Error! Objects cannot be created from editing field codes.

In analogy to example 53, step 4 using 1-[2-chloro-6-[5-[(6-methylpyridazin-3-yl)amino]benzimidazol-1-yl]-3-pyridyl]ethanone (200.0 mg, 0.530 mmol, 1.0 equiv. prepared in example 76, step 2) and 5.0 equivalents of NaBH4 1-[2-chloro-6-[5-[(6-methylpyridazin-3-yl)amino]benzimidazol-1-yl]-3-pyridyl]ethanol (130 mg, 0.340 mmol, 51.7% yield) was obtained as a grey solid. LC-MS: m/z=381.1, [M+H]⁺, ESI pos.

### Step 3: 1-[2-[4-(cyclopropylamino)pyrimidin-5-yl]-6-[5-[(6-methylpyridazin-3-yl)amino]benzimidazol-1-yl]-3-pyridyl]ethanol

Error! Objects cannot be created from editing field codes.

In analogy to example 143, step 2 using 1-[2-chloro-6-[5-[(6-methylpyridazin-3-yl)amino]benzimidazol-1-yl]-3-pyridyl]ethanol (30.0 mg, 0.080 mmol, 1.0 equiv. from Example 163, step 2) and [4-(cyclopropylamino)pyrimidin-5-yl]boronic acid (42.3 mg, 0.240 mmol, 3.0 equiv. from Example 163, step 1), 1-[2-[4-(cyclopropylamino)pyrimidin-5-yl]-6-[5-[(6-methylpyridazin-3-yl)amino]benzimidazol-1-yl]-3-pyridyl]ethanol (4 mg, 0.010 mmol, 10.5% yield) was obtained as white solid. LC-MS: m/z=480.1, [M+H]⁺, ESI pos.

### Example 164

### 5-[3-(1-Hydroxyethyl)-6-[5-[(6-methylpyridazin-3-yl)amino]benzimidazol-1-yl]-2-pyridyl]-4-(trifluoromethyl)pyridin-2-ol

Error! Objects cannot be created from editing field codes.

### Step 1: 5-bromo-2-methoxy-4-(trifluoromethyl)pyridine

Error! Objects cannot be created from editing field codes.

A solution of 5-bromo-2-chloro-4-(trifluoromethyl)pyridine (1.0 g, 3.84 mmol, 1.0 equiv.) and methoxysodium (0.41 g, 7.68 mmol, 2 equiv.) in methanol (7 mL) was stirred at 70° C. for 2 hour. The reaction mixture was dissolved in DCM (10 mL) and filtered, the filtrate was concentrated in vacuum to give the desired product as white solid, which was used without further purification. LC-MS: m/z=257.9, [M+H]⁺, ESI pos.

### Step 2: 2-methoxy-5-(4,4,5,5-tetramethyl-1,3,2-dioxaborolan-2-yl)-4-(trifluoromethyl)pyridine

Error! Objects cannot be created from editing field codes.

Prepared in analogy to example 143, step 2 3-bromo-6-methoxy-2-(trifluoromethyl)pyridine (used as crude from the previous step) to provide the desired compound as an off-white solid (400 mg, 62.7% yield). LC-MS: m/z=304.1, [M+H]⁺, ESI pos.

### Step 3: 1-[2-[6-methoxy-4-(trifluoromethyl)-3-pyridyl]-6-[5-[(6-methylpyridazin-3-yl)amino]benzimidazol-1-yl]-3-pyridyl]ethanone

Error! Objects cannot be created from editing field codes.

Prepared in analogy to example 143, step 2 using 2-methoxy-5-(4,4,5,5-tetramethyl-1,3,2-dioxaborolan-2-yl)-4-(trifluoromethyl)pyridine (240.03 mg, 0.790 mmol, 3 equiv.) and 1-[2-chloro-6-[5-[(6-methylpyridazin-3-yl)amino]benzimidazol-1-yl]-3-pyridyl]ethanone (100.0 mg, 0.260 mmol, 1.0 equiv., prepared in example 76, step 2) to give 1-[2-[6-methoxy-4-(trifluoromethyl)-3-pyridyl]-6-[5-[(6-methylpyridazin-3-yl)amino]benzimidazol-1-yl]-3-pyridyl]ethanone (45 mg, 0.090 mmol, 32.82% yield) as yellow solid. LC-MS: m/z=520.2, [M+H]⁺, ESI pos.

### Step 4: 1-[2-[6-hydroxy-4-(trifluoromethyl)-3-pyridyl]-6-[5-[(6-methylpyridazin-3-yl)amino]benzimidazol-1-yl]-3-pyridyl]ethanone; formic acid

Error! Objects cannot be created from editing field codes.

To a solution of 1-[2-[6-methoxy-4-(trifluoromethyl)-3-pyridyl]-6-[5-[(6-methylpyridazin-3-yl)amino]benzimidazol-1-yl]-3-pyridyl]ethanone (40.0 mg, 0.080 mmol, 1.0 equiv.) in HCl (2008.86 mg, 3 mmol, 39.0 equiv.) and the resulting mixture was stirred at 100° C. for 3 hours. The reaction mixture was concentrated under reduced pressure. The residue was purified by preparative HPLC (Phenomenex Luna C18 (150 mm×40 mm×15 μm). Flow rate: 60 mL/min. Gradient: 10% to 40% CH₃CN in (0.225% formic acid in H₂O v/v) (13 min) then 100% CH₃CN (2 min)) and lyophilized to yield 1-[2-[6-hydroxy-4-(trifluoromethyl)-3-pyridyl]-6-[5-[(6-methylpyridazin-3-yl)amino]benzimidazol-1-yl]-3-pyridyl]ethanone; formic acid (30 mg, 0.060 mmol, 77.1% yield) as yellow solid. LC-MS: m/z=506.1, [M+H]⁺, ESI pos.

### Step 5: 5-[3-(1-hydroxyethyl)-6-[5-[(6-methylpyridazin-3-yl)amino]benzimidazol-1-yl]-2-pyridyl]-4-(trifluoromethyl)pyridin-2-ol

Error! Objects cannot be created from editing field codes.

Prepared in analogy to example 53, step 4 to yield 5-[3-(1-hydroxyethyl)-6-[5-[(6-methylpyridazin-3-yl)amino]benzimidazol-1-yl]-2-pyridyl]-4-(trifluoromethyl)pyridin-2-ol (10.1 mg, 0.020 mmol, 32.7% yield) as grey solid. LC-MS: m/z=508.1, [M+H]⁺, ESI pos.

### Example 165

### 3-[3-(1-Hydroxyethyl)-6-[5-[(6-methylpyridazin-3-yl)amino]benzimidazol-1-yl]-2-pyridyl]-6,7-dihydro-5H-pyrazolo[1,5-a]pyrazin-4-one

Error! Objects cannot be created from editing field codes.

### Step 1: 6,7-dihydro-5H-pyrazolo[1,5-a]pyrazin-4-one

Error! Objects cannot be created from editing field codes.

To a solution of tert-butyl N-(2-bromoethyl)carbamate (10.66 g, 47.58 mmol, 1.2 equiv.) in DMF (100 mL) was added tert-butyl N-(2-bromoethyl)carbamate (10.66 g, 47.58 mmol, 1.2 equiv.) and Cs₂CO₃ (25.84 g, 79.3 mmol, 2 equiv.). The mixture was stirred at 30° C. for 12 hours. The mixture was filtered and the filter cake rinsed with EtOAc. The filtrate was concentrated under reduced pressure to remove EtOAc, diluted with water, and extracted with EtOAc. The combined extracts were washed with brine and concentrated under reduced pressure. The residue was purified by flash column chromatography (0-25% EtOAc in PE) and concentrated under reduced pressure to give colorless oil. This oil was dissolved in 1,4-dioxane (20 mL) 4M HCl in 1,4-dioxane (20.0 mL, 80 mmol, 2.02 equiv.) was added at 0° C. The mixture was stirred at 20° C. for 2 hours. The volatiles were removed, and the residue was dissolved in 1,4-dioxane (50 mL) and sodium carbonate (16.81 g, 158.59 mmol, 4 equiv.) was added as a solid. The mixture was stirred at 30° C. for 12 hours. The reaction mixture was filtered and the filter cake rinsed with EtOAc. The filtrate was concentrated under reduced pressure to remove the volatiles. The residue was purified by flash column chromatography (0-100% EtOAc in PE) to yield 6,7-dihydro-5H-pyrazolo[1,5-a]pyrazin-4-one (1.5 g, 10.94 mmol, 27.6% yield) as an orange powder. LC-MS: m/z=138.2, [M+H]⁺, ESI pos.

### Step 2: 3-bromo-6,7-dihydro-5H-pyrazolo[1,5-a]pyrazin-4-one

Error! Objects cannot be created from editing field codes.

To a solution of 6,7-dihydro-5H-pyrazolo[1,5-a]pyrazin-4-one (1.5 g, 10.94 mmol, 1.0 equiv.) in DMF (15 mL) was added 1-bromopyrrolidine-2,5-dione (2.14 g, 12.03 mmol, 1.1.0 equiv.). The mixture was stirred at 30° C. for 12 hours. The reaction mixture was poured into aq. sat. NH₄Cl and stirred for 10 min. Then mixture was filtered and rinsed with water (10 mL*2) and sat. Na₂S₂O₃ (10 mL). The filter cake was concentrated under reduced pressure yield 3-bromo-6,7-dihydro-5H-pyrazolo[1,5-a]pyrazin-4-one (2.2 g, 10.18 mmol, 93.1% yield) as an off-white solid. LC-MS: m/z=216.0, [M+H]⁺, ESI pos.

### Step 3: (4-oxo-6,7-dihydro-5H-pyrazolo[1,5-a]pyrazin-3-yl)boronic acid

Error! Objects cannot be created from editing field codes.

Prepared in analogy to example 143, step 2 using 3-bromo-6,7-dihydro-5H-pyrazolo[1,5-a]pyrazin-4-one (500.0 mg, 2.31 mmol, 1.0 equiv.) to yield (4-oxo-6,7-dihydro-5H-pyrazolo[1,5-a]pyrazin-3-yl)boronic acid (800 mg, 4.42 mmol, 48.61% yield) as a yellow solid. LC-MS: m/z=182.1, [M+H]⁺, ESI pos.

### Step 4: 3-[3-acetyl-6-[5-[(6-methylpyridazin-3-yl)amino]benzimidazol-1-yl]-2-pyridyl]-6,7-dihydro-5H-pyrazolo[1,5-a]pyrazin-4-one

Error! Objects cannot be created from editing field codes.

Prepared in analogy to example 143, step 2 (4-oxo-6,7-dihydro-5H-pyrazolo[1,5-a]pyrazin-3-yl)boronic acid (600.0 mg, 1.23 mmol, 7.75 equiv.) and 1-[2-chloro-6-[5-[(6-methylpyridazin-3-yl)amino]benzimidazol-1-yl]-3-pyridyl]ethanone ((60.0 mg, 0.160 mmol, 1.0 equiv., prepared in example 76, step 2) to yield 3-[3-acetyl-6-[5-[(6-methylpyridazin-3-yl)amino]benzimidazol-1-yl]-2-pyridyl]-6,7-dihydro-5H-pyrazolo[1,5-a]pyrazin-4-one (35 mg, 0.070 mmol, 45.6% yield) as a brown solid. LC-MS: m/z=480.2, [M+H]⁺, ESI pos.

### Step 5: 3-[3-(1-hydroxyethyl)-6-[5-[(6-methylpyridazin-3-yl)amino]benzimidazol-1-yl]-2-pyridyl]-6,7-dihydro-5H-pyrazolo[1,5-a]pyrazin-4-one

Error! Objects cannot be created from editing field codes.

Prepared in analogy to example 53, step 4 to give the 3-[3-(1-hydroxyethyl)-6-[5-[(6-methylpyridazin-3-yl)amino]benzimidazol-1-yl]-2-pyridyl]-6,7-dihydro-5H-pyrazolo[1,5-a]pyrazin-4-one (6.8 mg, 0.010 mmol, 22.6% yield) as a white solid. LC-MS: m/z=482.2, [M+H]⁺, ESI pos.

### Example 166

### 1-[6-[6-Fluoro-5-[[(3S,4R)-4-fluoropyrrolidin-3-yl]amino]benzimidazol-1-yl]-3-(1-hydroxyethyl)-2-pyridyl]-5-methyl-pyrazole-3-carbonitrile; formic acid

Error! Objects cannot be created from editing field codes.

### Step 1: tert-butyl (3R,4S)-3-fluoro-4-(2-fluoro-4,5-dinitro-anilino)pyrrolidine-1-carboxylate

Error! Objects cannot be created from editing field codes.

To a solution of 1,2-difluoro-4,5-dinitro-benzene (450.0 mg, 2.2 mmol, 1.0 equiv.) in DMF (10 mL) was added tert-butyl (3S,4R)-3-amino-4-fluoropyrrolidine-1-carboxylate (450.0 mg, 2.2 mmol, 1.0 equiv.) and N,N-Diisopropylethylamine (1.09 mL, 6.61 mmol, 3.0 equiv.). The reaction mixture was stirred at 30° C. for 12 hours. The reaction mixture was diluted with water, and extracted with EtOAc. The organic layers were washed with brine and concentrated under vacuum to yield tert-butyl (3R,4S)-3-fluoro-4-(2-fluoro-4,5-dinitro-anilino)pyrrolidine-1-carboxylate (800 mg, 2.06 mmol, 93.4% yield) as yellow solid. LC-MS: m/z=289.1 [M-Boc+H]⁺, ESI pos.

### Step 2: 6-fluoro-N-[(3S,4R)-4-fluoropyrrolidin-3-yl]-1H-benzimidazol-5-amine

Error! Objects cannot be created from editing field codes.

A solution of tert-butyl (3R,4S)-3-fluoro-4-(2-fluoro-4,5-dinitro-anilino)pyrrolidine-1-carboxylate (550.0 mg, 1.42 mmol, 1.0 equiv.) in formic acid (65.19 mg, 1.42 mmol, 1.0 equiv.) was added nickel (83.13 mg, 1.42 mmol, 1.0 equiv.). The mixture was stirred at 30° C. for 12 h under H₂ atmosphere (50 psi). The mixture was filtered through a pad of celite and the filtrate was concentrated in vacuo to yield crude 6-fluoro-N-[(3S,4R)-4-fluoropyrrolidin-3-yl]-1H-benzimidazol-5-amine (500 mg, 2.1 mmol, 74.1% yield) as dark brown gum. LC-MS: m/z=239.1 [M+H]⁺, ESI pos.

### Step 3: tert-butyl (3R,4S)-3-fluoro-4-[(6-fluoro-1H-benzimidazol-5-yl)amino]pyrrolidine-1-carboxylate

Error! Objects cannot be created from editing field codes.

To a solution of 6-fluoro-N-[(3S,4R)-4-fluoropyrrolidin-3-yl]-1H-benzimidazol-5-amine (600.0 mg, 1.26 mmol, 1.0 equiv.) in DCM (10 mL) and was added TEA (382.27 mg, 3.78 mmol, 3 equiv.) and tert-butyl (2-methylpropan-2-yl)oxycarbonyl carbonate (412.24 mg, 1.89 mmol, 1.5 equiv.). The mixture was stirred at 25° C. for 12 hours. The mixture was poured into water and extracted with EtOAc. The combined organic layers were washed with brine, dried over Na₂SO₄, filtered and the filtrate was concentrated in vacuo. The residue was dissolved in MeOH, and to the solution was added sat. aq.

K₂CO₃. The mixture was stirred at 25° C. for 12 hours. The mixture was extracted with EtOAc and the combined organic layers were dried over Na₂SO₄, filtered and the filtrate was concentrated in vacuo. The residue was purified by prep-HPLC (Waters Xbridge C18 150 mm×50 mm×10 μm, gradient 3-33% CH₃CN in H₂O (with 10 mM NH₄CO₃) over 11 min, then 100% CH₃CN (2 min), flow rate 60 mL/min)), and the eluent was lyophilized to yield tert-butyl (3R,4S)-3-fluoro-4-[(6-fluoro-1H-benzimidazol-5-yl)amino]pyrrolidine-1-carboxylate (180 mg, 0.530 mmol, 42.3% yield) as off-white solid. LC-MS: m/z=339.2 [M+H]⁺, ESI pos.

### Step 4: tert-butyl (3S,4R)-3-[[1-[5-acetyl-6-(3-cyano-5-methyl-pyrazol-1-yl)-2-pyridyl]-6-fluoro-benzimidazol-5-yl]amino]-4-fluoro-pyrrolidine-1-carboxylate

Error! Objects cannot be created from editing field codes.

Prepared in analogy to example 64, step 2 using tert-butyl rac-(3R,4S)-3-fluoro-4-[(6-fluoro-1H-benzimidazol-5-yl)amino]pyrrolidine-1-carboxylate (170.0 mg, 0.500 mmol, 1.0 equiv.) and 1-(3-acetyl-6-chloro-2-pyridyl)-5-methyl-pyrazole-3-carbonitrile (196.46 mg, 0.750 mmol, 1.5 equiv., prepared in Example 64, step 1) to yield tert-butyl (3S,4R)-3-[[3-[5-acetyl-6-(3-cyano-5-methyl-pyrazol-1-yl)-2-pyridyl]-6-fluoro-benzimidazol-5-yl]amino]-4-fluoro-pyrrolidine-1-carboxylate (40 mg, 0.070 mmol, 14.2% yield) and tert-butyl (3S,4R)-3-[[1-[5-acetyl-6-(3-cyano-5-methyl-pyrazol-1-yl)-2-pyridyl]-6-fluoro-benzimidazol-5-yl]amino]-4-fluoro-pyrrolidine-1-carboxylate (50 mg, 0.090 mmol, 17.7% yield) as light yellow gums. The regioisomers were separated by preparative NPLC (Welch Ultimate XB-SiOH 250×50×10 μm, 10-50% EtOH (0.1% ammonium hydroxide) in hexane over 15 min, then 100% EtOH (0.1% ammonium hydroxide) (5 min), flow rate 100 mL/min). LC-MS: m/z=563.2 [M+H]⁺, ESI pos. Regioisomer 1: ¹H NMR (400 MHz, CDCl₃) δ=8.52 (s, 1H), 8.28 (d, J=8.4 Hz, 1H), 7.78 (d, J=11.1 Hz, 1H), 7.68 (d, J=8.4 Hz, 1H), 7.18 (d, J=7.8 Hz, 1H), 6.73 (s, 1H), 5.36-5.13 (m, 1H), 4.41 (br d, J=7.8 Hz, 1H), 4.27-4.02 (m, 2H), 3.73 (q, J=6.9 Hz, 1H), 3.21 (q, J=9.7 Hz, 1H), 2.61 (s, 3H), 2.20 (s, 3H), 1.49 (s, 9H). Regioisomer 2: ¹H NMR (400 MHz, CDCl₃) δ=8.45-8.37 (m, 1H), 8.32 (d, J=8.3 Hz, 1H), 7.70 (br d, J=8.3 Hz, 1H), 7.51 (br d, J=11.1 Hz, 1H), 7.40 (d, J=7.5 Hz, 1H), 6.76-6.67 (m, 1H), 5.28-5.04 (m, 1H), 4.64-4.40 (m, 1H), 4.04-3.53 (m, 4H), 3.27-3.11 (m, 1H), 2.58 (s, 3H), 2.26-2.17 (s, 3H), 1.54-1.46 (s, 9H).

### Step 5: 1-[3-acetyl-6-[6-fluoro-5-[[(3S,4R)-4-fluoropyrrolidin-3-yl]amino]benzimidazol-1-yl]-2-pyridyl]-5-methyl-pyrazole-3-carbonitrile

Error! Objects cannot be created from editing field codes.

A solution of tert-butyl (3S,4R)-3-[[1-[5-acetyl-6-(3-cyano-5-methyl-pyrazol-1-yl)-2-pyridyl]-6-fluoro-benzimidazol-5-yl]amino]-4-fluoro-pyrrolidine-1-carboxylate (50.0 mg, 0.090 mmol, 1.0 equiv.) and trifluoroacetic acid (0.63 mL, 8.11 mmol, 91.3 equiv.) in DCM (2.5 mL) was stirred at 25° C. for 1 hour. The mixture was concentrated in vacuo to yield 60 mg of crude 1-[3-acetyl-6-[6-fluoro-5-[[(3S,4R)-4-fluoropyrrolidin-3-yl]amino]benzimidazol-1-yl]-2-pyridyl]-5-methyl-pyrazole-3-carbonitrile. The crude product was used in next step without further purification. LC-MS: m/z=463.1 [M+H]⁺, ESI pos.

### Step 6: 1-[6-[6-fluoro-5-[[(3S,4R)-4-fluoropyrrolidin-3-yl]amino]benzimidazol-1-yl]-3-(1-hydroxyethyl)-2-pyridyl]-5-methyl-pyrazole-3-carbonitrile; formic acid

Error! Objects cannot be created from editing field codes.

To a solution of 1-[3-acetyl-6-[6-fluoro-5-[[(3S,4R)-4-fluoropyrrolidin-3-yl]amino]benzimidazol-1-yl]-2-pyridyl]-5-methyl-pyrazole-3-carbonitrile; 2,2,2-trifluoroacetic acid (used as crude from the previous step) in methanol (3 mL) was added NaBH₄ (11.81 mg, 0.310 mmol, 3.0 equiv.) at 0° C. and stirred for 30 min. The mixture was quenched by sat. aq. NH₄Cl and extracted with EtOAc. The combined organic layers were dried over Na₂SO₄ and was concentrated in vacuo. The residue was purified by preparative HPLC (Waters Xbridge 150*25 mm*5 um, H₂O (10 mM NH₄HCO₃)—CH₃CN, 23-53% B, gradient time 8 min, 100% B hold time 2 min, flow rate 25 mL/min), the eluent was lyophilized to yield 1-[6-[6-fluoro-5-[[(3S,4R)-4-fluoropyrrolidin-3-yl]amino]benzimidazol-1-yl]-3-(1-hydroxyethyl)-2-pyridyl]-5-methyl-pyrazole-3-carbonitrile; formic acid (13.6 mg, 0.030 mmol, 26.73% yield) as white solid. LC-MS: m/z=465.3 [M+H]⁺, ESI pos.

### Example 167

### 1-[2-[2-(Difluoromethoxy)-5-methyl-4-pyridyl]-6-[5-[(6-methylpyridazin-3-yl)amino]benzimidazol-1-yl]-3-pyridyl]ethanol

Error! Objects cannot be created from editing field codes.

### Step 1: 4-bromo-2-(difluoromethoxy)-5-methyl-pyridine

Error! Objects cannot be created from editing field codes.

A mixture of 4-bromo-5-methyl-pyridin-2-ol (290.0 mg, 1.54 mmol, 1.0 equiv.), Cs₂CO₃ (1.01 g, 3.08 mmol, 2.0 equiv.) and sodium chlorodifluoroacetate (705.46 mg, 4.63 mmol, 3.0 equiv.) in DMF (5 mL) was stirred at 100° C. for 12 hours. The reaction mixture was filtered, the filtrate was dissolved in brine, extracted with ethyl acetate, and the combined extracts were concentrated under reduced pressure. The residue was purified by flash column chromatography (9% EtOAc in PE) to yield 4-bromo-2-(difluoromethoxy)-5-methyl-pyridine (200 mg, 0.840 mmol, 54.48% yield) as a colorless oil. LC-MS: m/z=238.0, [M+H]⁺, ESI pos.

### Step 2: 2-(difluoromethoxy)-5-methyl-4-(4,4,5,5-tetramethyl-1,3,2-dioxaborolan-2-yl)pyridine

Error! Objects cannot be created from editing field codes.

Prepared in analogy to example 143, step 2 using 4-bromo-2-(difluoromethoxy)-5-methyl-pyridine (150.0 mg, 0.630 mmol, 1.0 equiv) to yield 2-(difluoromethoxy)-5-methyl-4-(4,4,5,5-tetramethyl-1,3,2-dioxaborolan-2-yl)pyridine (250 mg, 0.880 mmol, 139.2% yield) as a yellow oil. LC-MS: m/z=286.2, [M+H]⁺, ESI pos.

### Step 3: 1-[2-[2-(difluoromethoxy)-5-methyl-4-pyridyl]-6-[5-[(6-methylpyridazin-3-yl)amino]benzimidazol-1-yl]-3-pyridyl]ethanone

Error! Objects cannot be created from editing field codes.

Prepared in analogy to example 143, step 2 using 1-[2-chloro-6-[5-[(6-methylpyridazin-3-yl)amino]benzimidazol-1-yl]-3-pyridyl]ethanone (50.0 mg, 0.130 mmol, 1.0 equiv., prepared in example 76, step 2), and 2-(difluoromethoxy)-5-methyl-4-(4,4,5,5-tetramethyl-1,3,2-dioxaborolan-2-yl)pyridine (718.5 mg, 2.5 mmol, 20 equiv.) to afford 1-[2-[2-(difluoromethoxy)-5-methyl-4-pyridyl]-6-[5-[(6-methylpyridazin-3-yl)amino]benzimidazol-1-yl]-3-pyridyl]ethanone (30 mg, 0.060 mmol, 45.3% yield) as a yellow solid. LC-MS: m/z=502.3, [M+H]⁺, ESI pos.

### Step 4: 1-[2-[2-(difluoromethoxy)-5-methyl-4-pyridyl]-6-[5-[(6-methylpyridazin-3-yl)amino]benzimidazol-1-yl]-3-pyridyl]ethanol

Error! Objects cannot be created from editing field codes.

Prepared in analogy to example 53, step 4 using 5.0 equivalents of NaBH₄ (11.37 mg, 0.300 mmol) to yield 1-[2-[2-(difluoromethoxy)-5-methyl-4-pyridyl]-6-[5-[(6-methylpyridazin-3-yl)amino]benzimidazol-1-yl]-3-pyridyl]ethanol (19.4 mg, 0.040 mmol, 64.4% yield) as a yellow solid. LC-MS: m/z=502.4, [M+H]⁺, ESI pos.

### Example 168

### 1-[2-(2-Fluoro-5-methyl-4-pyridyl)-6-[5-[(6-methylpyridazin-3-yl)amino]benzimidazol-1-yl]-3-pyridyl]ethanol

Error! Objects cannot be created from editing field codes.

### Step 1: 2-fluoro-5-methyl-4-(4,4,5,5-tetramethyl-1,3,2-dioxaborolan-2-yl)pyridine

Error! Objects cannot be created from editing field codes.

Prepared in analogy to example 143, step 2 using 4-bromo-2-fluoro-5-methyl-pyridine (950.0 mg, 5 mmol, 1.0 equiv.) to afford 2-fluoro-5-methyl-4-(4,4,5,5-tetramethyl-1,3,2-dioxaborolan-2-yl)pyridine (1000 mg, 4.22 mmol, 84.4% yield) as light yellow solid. LC-MS: m/z=238.1 [M+H]⁺, ESI pos. ¹H NMR (400 MHz, CDCl₃) δ=8.00 (s, 1H), 7.22 (s, 1H), 2.44 (s, 3H), 1.35 (s, 13H).

### Step 2: 1-[2-(2-fluoro-5-methyl-4-pyridyl)-6-[5-[(6-methylpyridazin-3-yl)amino]benzimidazol-1-yl]-3-pyridyl]ethanone

Error! Objects cannot be created from editing field codes.

Prepared in analogy to example 143, step 2 using 1-[2-chloro-6-[5-[(6-methylpyridazin-3-yl)amino]benzimidazol-1-yl]-3-pyridyl]ethanone (50.0 mg, 0.130 mmol, 1.0 equiv., prepared in example 76, step 2) and 2-fluoro-5-methyl-4-(4,4,5,5-tetramethyl-1,3,2-dioxaborolan-2-yl)pyridine (125.17 mg, 0.530 mmol, 4.0 equiv.) to yield 1-[2-(2-fluoro-5-methyl-4-pyridyl)-6-[5-[(6-methylpyridazin-3-yl)amino]benzimidazol-1-yl]-3-pyridyl]ethanone (30 mg, 0.070 mmol, 50.1% yield) as grey solid. LC-MS: m/z=454.1 [M+H]⁺, ESI pos.

### Step 3: 1-[2-(2-fluoro-5-methyl-4-pyridyl)-6-[5-[(6-methylpyridazin-3-yl)amino]benzimidazol-1-yl]-3-pyridyl]ethanol

Error! Objects cannot be created from editing field codes.

Prepared in analogy to example 53, step 4 using 5.0 equivalents of NaBH₄ (11.26 mg, 0.300 mmol, 5 equiv.) LC-MS: m/z=456.2 [M+H]⁺, ESI pos.

### Example 169

### 1-[3-(1-Hydroxyethyl)-6-[6-[(6-methylpyridazin-3-yl)amino]benzimidazol-1-yl]-2-pyridyl]-4-methyl-pyrazole-3-carbonitrile; formic acid

Error! Objects cannot be created from editing field codes.

### Step 1: 1-(3-acetyl-6-chloro-2-pyridyl)-4-bromo-pyrazole-3-carbonitrile

Error! Objects cannot be created from editing field codes.

Prepared in analogy to example 62, step 1 using 4-bromopyrazole-3-carbonitrile (1.0 g, 5.81 mmol, 1.0 equiv.) to afford 1-(3-acetyl-6-chloro-2-pyridyl)-4-bromo-pyrazole-3-carbonitrile (1.5 g, 4.61 mmol, 79.2% yield) as off-white solid. LC-MS: m/z=326.8 [M+H]⁺, ESI pos.

### Step 2: 1-[3-acetyl-6-[6-[(6-methylpyridazin-3-yl)amino]benzimidazol-1-yl]-2-pyridyl]-4-bromo-pyrazole-3-carbonitrile and 1-[3-acetyl-6-[5-[(6-methylpyridazin-3-yl)amino]benzimidazol-1-yl]-2-pyridyl]-4-bromo-pyrazole-3-carbonitrile

Error! Objects cannot be created from editing field codes.

Prepared in analogy to example 76, step 1 using 1-(3-acetyl-6-chloro-2-pyridyl)-4-bromo-pyrazole-3-carbonitrile (1.0 g, 3.07 mmol, 1.0 equiv.) and N-(6-methylpyridazin-3-yl)-1H-benzimidazol-5-amine (0.83 g, 3.69 mmol, 1.2 equiv., prepared in example 64, step 1) to provide 1-[3-acetyl-6-[6-[(6-methylpyridazin-3-yl)amino]benzimidazol-1-yl]-2-pyridyl]-4-bromo-pyrazole-3-carbonitrile (350 mg, 0.680 mmol, 22.2% yield) and 1-[3-acetyl-6-[5-[(6-methylpyridazin-3-yl)amino]benzimidazol-1-yl]-2-pyridyl]-4-bromo-pyrazole-3-carbonitrile (160 mg, 0.310 mmol, 10.1% yield) as an light brown solid. Regioisomer 1: LC-MS: m/z=514.0 [M+H]⁺, ESI pos. ¹H NMR (400 MHz, DMSO-d₆) δ=9.32 (s, 1H), 9.29 (s, 1H), 9.17 (s, 1H), 8.44 (d, J=1.7 Hz, 1H), 8.40 (d, J=8.4 Hz, 1H), 8.21 (dd, J=8.7, 10.7 Hz, 2H), 7.61 (dd, J=1.8, 8.8 Hz, 1H), 7.35 (d, J=9.2 Hz, 1H), 7.10 (d, J=9.0 Hz, 1H), 2.49 (s, 3H), 2.41 (s, 3H). Regioisomer 2: LC-MS: m/z=514.0 [M+H]⁺, ESI pos. ¹H NMR (400 MHz, DMSO-d₆) δ=9.64 (d, J=1.7 Hz, 1H), 9.45 (s, 1H), 9.39 (s, 1H), 8.99 (s, 1H), 8.40 (d, J=8.4 Hz, 1H), 8.20-8.13 (m, 1H), 7.68 (d, J=8.7 Hz, 1H), 7.37 (d, J=9.0 Hz, 1H), 7.25 (dd, J=1.9, 8.7 Hz, 1H), 7.15 (d, J=9.0 Hz, 1H), 2.51 (br d, J=1.5 Hz, 3H), 2.43 (s, 2H).

### Step 3: 1-[3-acetyl-6-[6-[(6-methylpyridazin-3-yl)amino]benzimidazol-1-yl]-2-pyridyl]-4-methyl-pyrazole-3-carbonitrile

Error! Objects cannot be created from editing field codes.

Prepared in analogy to example 169/170, step 3 using 1-[3-acetyl-6-[6-[(6-methylpyridazin-3-yl)amino]benzimidazol-1-yl]-2-pyridyl]-4-bromo-pyrazole-3-carbonitrile (50.0 mg, 0.100 mmol, 1.0 equiv.) to afford the title compound as an off-white solid (25.1 mg, 0.056 mmol, 56.2% yield). LC-MS: m/z=450.1, [M+H]⁺, ESI pos.

### Step 4: 1-[3-(-hydroxyethyl)-6-[6-[(6-methylpyridazin-3-yl)amino]benzimidazol-1-yl]-2-pyridyl]-4-methyl-pyrazole-3-carbonitrile; formic acid

Error! Objects cannot be created from editing field codes.

Prepared in analogy to example 53, step 4 using 1-[3-acetyl-6-[6-[(6-methylpyridazin-3-yl)amino]benzimidazol-1-yl]-2-pyridyl]-4-methyl-pyrazole-3-carbonitrile (20.0 mg, 0.040 mmol, 1.0 equiv.) in MeOH/DCM (5:1) to provide 1-[3-(1-hydroxyethyl)-6-[6-[(6-methylpyridazin-3-yl)amino]benzimidazol-1-yl]-2-pyridyl]-4-methyl-pyrazole-3-carbonitrile; formic acid (3.8 mg, 0.010 mmol, 14.9% yield) as yellow solid by lyophilization. LC-MS: m/z=452.2, [M+H]⁺, ESI pos. ¹H NMR (400 MHz, CD₃OD) δ=9.31 (d, J=1.7 Hz, 1H), 8.86-8.81 (m, 1H), 8.71 (s, 1H), 8.57 (d, J=8.4 Hz, 1H), 8.44 (br s, 2H), 8.03-7.98 (m, 1H), 7.69 (d, J=8.8 Hz, 1H), 7.40 (d, J=9.2 Hz, 1H), 7.30 (dd, J=1.8, 8.6 Hz, 1H), 7.20-7.15 (m, 1H), 5.61 (q, J=6.3 Hz, 1H), 2.58 (s, 3H), 2.21 (s, 3H), 1.54 (d, J=6.4 Hz, 3H).

### Example 170

### 1-[3-(1-Hydroxyethyl)-6-[5-[(6-methylpyridazin-3-yl)amino]benzimidazol-1-yl]-2-pyridyl]-4-methyl-pyrazole-3-carbonitrile; formic acid

Error! Objects cannot be created from editing field codes.

### Step 1: 1-[3-acetyl-6-[5-[(6-methylpyridazin-3-yl)amino]benzimidazol-1-yl]-2-pyridyl]-4-methyl-pyrazole-3-carbonitrile

Error! Objects cannot be created from editing field codes.

To a solution of 1-[3-acetyl-6-[5-[(6-methylpyridazin-3-yl)amino]benzimidazol-1-yl]-2-pyridyl]-4-bromo-pyrazole-3-carbonitrile (200.0 mg, 0.390 mmol, 1.0 equiv., provided in example 169, step 2) and trimethylboroxine (195.25 mg, 0.780 mmol, 2.0 equiv.) in DMF (15 mL) were added K₃PO₄ (0.13 mL, 1.56 mmol, 4.0 equiv.) and PdCl₂(dppf) CH₂Cl₂ (31.73 mg, 0.040 mmol, 0.10 equiv.). The mixture was purged with nitrogen for several min, and then the reaction was stirred at 100° C. for 12 h under nitrogen atmosphere. The mixture was poured into water and extracted with EtOAc. The organic layers were separated, dried over Na₂SO₄, filtered and concentracted under reduce pressure. The crude was purified by preparative TLC (10% MeOH in DCM) to provide 1-[3-acetyl-6-[5-[(6-methylpyridazin-3-yl)amino]benzimidazol-1-yl]-2-pyridyl]-4-methyl-pyrazole-3-carbonitrile (40 mg, 0.090 mmol, 22.9% yield) as off white solid. LC-MS: m/z=450.2, [M+H]⁺, ESI pos.

### Step 2: 1-[3-(-hydroxyethyl)-6-[5-[(6-methylpyridazin-3-yl)amino]benzimidazol-1-yl]-2-pyridyl]-4-methyl-pyrazole-3-carbonitrile; formic acid

Error! Objects cannot be created from editing field codes.

Prepared in analogy to example 53, step 4 using 1-[3-acetyl-6-[5-[(6-methylpyridazin-3-yl)amino]benzimidazol-1-yl]-2-pyridyl]-4-methyl-pyrazole-3-carbonitrile (40.0 mg, 0.090 mmol, 1.0 equiv.) in MeOH/DCM (5:1) to provide 1-[3-(1-hydroxyethyl)-6-[5-[(6-methylpyridazin-3-yl)amino]benzimidazol-1-yl]-2-pyridyl]-4-methyl-pyrazole-3-carbonitrile; formic acid (10.3 mg, 0.020 mmol, 23.3% yield) as yellow solid. LC-MS: m/z=452.2, [M+H]⁺, ESI pos. ¹H NMR (400 MHz, CD₃OD) δ=8.93 (s, 1H), 8.53 (d, J=8.4 Hz, 1H), 8.49 (s, 1H), 8.41 (br s, 1H), 8.24 (d, J=1.8 Hz, 1H), 8.17 (d, J=8.8 Hz, 1H), 8.02 (d, J=8.3 Hz, 1H), 7.67 (dd, J=1.9, 9.0 Hz, 1H), 7.38 (d, J=9.0 Hz, 1H), 7.15 (d, J=9.2 Hz, 1H), 5.53 (q, J=6.0 Hz, 1H), 2.56 (s, 3H), 2.36 (s, 3H), 1.52 (d, J=6.4 Hz, 3H).

### Example 171

### 1-[6-[5-[(6-Methylpyridazin-3-yl)amino]benzimidazol-1-yl]-2-[5-methyl-3-(trifluoromethyl)-6,7-dihydro-4H-pyrazolo[4,3-c]pyridin-1-yl]-3-pyridyl]ethanol; formic acid

Error! Objects cannot be created from editing field codes.

### Step 1: 1-[6-[5-[(6-methylpyridazin-3-yl)amino]benzimidazol-1-yl]-2-[5-methyl-3-(trifluoromethyl)-6,7-dihydro-4H-pyrazolo[4,3-c]pyridin-1-yl]-3-pyridyl]ethanone

Error! Objects cannot be created from editing field codes.

A solution of 1-[6-[5-[(6-methylpyridazin-3-yl)amino]benzimidazol-1-yl]-2-[3-(trifluoromethyl)-4,5,6,7-tetrahydropyrazolo[4,3-c]pyridin-1-yl]-3-pyridyl]ethanone; hydrochloride (300.0 mg, 0.530 mmol, 1.0 equiv., prepared in example 97, step 3) and formaldehyde (50.0 mg, 1.67 mmol, 3.2 equiv.) in MeOH (10 mL) was stirred at 30° C. for 30 min. Then NaBH₃CN (100.0 mg, 1.59 mmol, 3.0 equiv.) was added to the mixture which was further stirred at 30° C. for 1.5 hours. The reaction mixture was diluted with water and extracted with EtOAc. The combined organic layers were dried over Na₂SO₄, filtered and concentrated under reduced pressure to yield crude 1-[6-[5-[(6-methylpyridazin-3-yl)amino]benzimidazol-1-yl]-2-[5-methyl-3-(trifluoromethyl)-6,7-dihydro-4H-pyrazolo[4,3-c]pyridin-1-yl]-3-pyridyl]ethanone (183 mg, 0.334 mmol, 63.1% yield) as yellow solid. The crude product was used without further purification. LC-MS: m/z=548.3 [M+H]⁺, ESI pos.

### Step 2: 1-[6-[5-[(6-methylpyridazin-3-yl)amino]benzimidazol-1-yl]-2-[5-methyl-3-(trifluoromethyl)-6,7-dihydro-4H-pyrazolo[4,3-c]pyridin-1-yl]-3-pyridyl]ethanol; formic acid

Error! Objects cannot be created from editing field codes.

Prepared in analogy to example 53, step 4 in DCM/MeOH (9:1) using 1-[6-[5-[(6-methylpyridazin-3-yl)amino]benzimidazol-1-yl]-2-[5-methyl-3-(trifluoromethyl)-6,7-dihydro-4H-pyrazolo[4,3-c]pyridin-1-yl]-3-pyridyl]ethanone (183 mg, 0.334 mmol, 1.0 eq.) to yield 1-[6-[5-[(6-methylpyridazin-3-yl)amino]benzimidazol-1-yl]-2-[5-methyl-3-(trifluoromethyl)-6,7-dihydro-4H-pyrazolo[4,3-c]pyridin-1-yl]-3-pyridyl]ethanol; formic acid (58 mg, 0.106 mmol, 31.8% yield) as light yellow solid. LC-MS: m/z=550.2 [M+H]⁺, ESI pos.

### Example 172

### 1-[[[3-(1-hydroxyethyl)-6-[6-[(6-methylpyridazin-3-yl)amino]benzimidazol-1-yl]-2-pyridyl]-methyl-amino]methyl]cyclopropanecarbonitrile

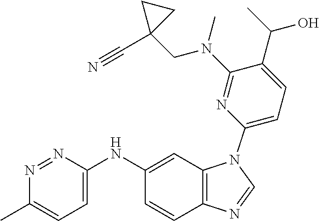 [SMILES: Cc1ccc(Nc2ccc3ncn(-c4ccc(C(C)O)c(N(C)CC5(C#N)CC5)n4)c3c2)nn1]

### Step 1: tert-butyl N-[(1-cyanocyclopropyl)methyl]carbamate

Error! Objects cannot be created from editing field codes.

To a solution of 1-(aminomethyl)cyclopropanecarbonitrile; hydrochloride (450.0 mg, 3.39 mmol, 1.0 equiv.) in DCM (15 mL) was added tert-butyl (2-methylpropan-2-yl)oxycarbonyl carbonate (962.9 mg, 4.41 mmol, 1.3 equiv.) and TEA (1.03 g, 10.18 mmol, 3.0 equiv.). The mixture was stirred at 30° C. for 12 hours. The mixture was diluted with DCM and purified by flash column chromatography (10-90% MeOH in DCM) to yield tert-butyl N-[(1-cyanocyclopropyl)methyl]carbamate (350 mg, 1.78 mmol, 52.6% yield) as an off-white solid. ¹H NMR (400 MHz, CDCl₃) δ=3.25 (d, J=6.4 Hz, 2H), 1.46 (s, 9H), 1.29-1.22 (m, 2H), 1.06 (s, 2H).

### Step 2: tert-butyl N-[(-cyanocyclopropyl)methyl]-N-methyl-carbamate

Error! Objects cannot be created from editing field codes.

To a solution of tert-butyl N-[(1-cyanocyclopropyl)methyl]carbamate (680.0 mg, 3.46 mmol, 1.0 equiv.) in THF (15 mL) und inert atmosphere was added NaH (207.9 mg, 5.2 mmol, 1.5 equiv.) at 0° C. The mixture was stirred at 0° C. for 30 min. Then Mel (737.7 mg, 5.2 mmol, 1.5 equiv.) was added. The mixture was warmed to 25° C. and stirred for 12 hours. The mixture was quenched by sat. aq. NH₄Cl and extracted with EtOAc. The combined organic layers were dried over Na₂SO₄ and the volatiles evaporated to yield crude tert-butyl N-[(1-cyanocyclopropyl)methyl]-N-methyl-carbamate as yellow oil which was used without further purification in the next step.

### Step 3: 1-(methylaminomethyl)cyclopropanecarbonitrile; 2,2,2-trifluoroacetic acid

Error! Objects cannot be created from editing field codes.

A solution of tert-butyl N-[(1-cyanocyclopropyl)methyl]-N-methyl-carbamate (used as crude from the previous step) and TFA (5.0 mL, 64.9 mmol, 20 equiv.) in DCM (5 mL) was stirred at 30° C. for 12 hours. The mixture was concentrated in vacuo to give the crude product 1-(methylaminomethyl)cyclopropanecarbonitrile; 2,2,2-trifluoroacetic as light yellow gum which was used in the next step without further purification. LC-MS: m/z=111.1 [M+H]⁺, ESI pos.

### Step 4: 1-[[(3-acetyl-6-chloro-2-pyridyl)-methyl-amino]methyl]cyclopropanecarbonitrile

Error! Objects cannot be created from editing field codes.

To a solution of 1-(6-chloro-2-fluoro-3-pyridyl)ethanone (CAS #1260663-13-5, 696.8 mg, 4.01 mmol, 1.2 equiv.), 1-(methylaminomethyl)cyclopropanecarbonitrile; 2,2,2-trifluoroacetic acid (used as crude from the previous step) in DMSO (8 mL) was added DIPEA (1.66 mL, 10.04 mmol, 3.0 equiv.) and was stirred at 30° C. for 12 hours. The mixture was diluted with water and extracted with EtOAc. The combined organic layers were washed with brine, dried over Na₂SO₄, and the volatiles evaporated. The residue was purified by flash column chromatography (10-33% EtOAc in PE) to yield 1-[[(3-acetyl-6-chloro-2-pyridyl)-methyl-amino]methyl]cyclopropanecarbonitrile (650 mg, 2.46 mmol, 70.7% yield) as a colorless gum. LC-MS: m/z=254.0 [M+H]⁺, ESI pos.

### Step 5: 1-[[[3-acetyl-6-[6-[(6-methylpyridazin-3-yl)amino]benzimidazol-1-yl]-2-pyridyl]-methyl-amino]methyl]cyclopropanecarbonitrile and 1-[[[3-acetyl-6-[5-[(6-methylpyridazin-3-yl)amino]benzimidazol-1-yl]-2-pyridyl]-methyl-amino]methyl]cyclopropanecarbonitrile

Error! Objects cannot be created from editing field codes.

Prepared in analogy to example 64, step 2 using 1-[[(3-acetyl-6-chloro-2-pyridyl)-methyl-amino]methyl]cyclopropanecarbonitrile (550.0 mg, 2.09 mmol, 1.0 equiv.) and N-(6-methylpyridazin-3-yl)-1H-benzimidazol-5-amine (704.65 mg, 3.13 mmol, 1.5 equiv., prepared in example 64, intermediate 1) to yield 1-[[[3-acetyl-6-[6-[(6-methylpyridazin-3-yl)amino]benzimidazol-1-yl]-2-pyridyl]-methyl-amino]methyl]cyclopropanecarbonitrile (110 mg, 0.240 mmol, 11.7% yield) as yellow solid and 1-[[[3-acetyl-6-[5-[(6-methylpyridazin-3-yl)amino]benzimidazol-1-yl]-2-pyridyl]-methyl-amino]methyl]cyclopropanecarbonitrile (110 mg, 0.240 mmol, 11.7% yield) as brown solid. Regioisomer 1: LC-MS: m/z=453.2 [M+H]⁺, ESI pos. ¹H NMR (400 MHz, DMSO-d₆) δ=9.33 (br s, 2H), 8.86 (s, 1H), 8.21 (d, J=8.2 Hz, 1H), 7.67 (d, J=8.7 Hz, 1H), 7.36 (d, J=9.0 Hz, 1H), 7.29 (d, J=8.2 Hz, 1H), 7.25 (dd, J=1.7, 8.9 Hz, 1H), 7.13 (d, J=9.0 Hz, 1H), 4.02 (s, 2H), 3.02 (s, 3H), 2.62 (s, 3H), 2.53-2.51 (m, 3H), 1.22-1.16 (m, 2H), 1.02-0.97 (m, 2H). Regioisomer 2: LC-MS: m/z=453.2 [M+H]⁺, ESI pos. ¹H NMR (400 MHz, DMSO-d₆) δ=9.23 (s, 1H), 8.98 (s, 1H), 8.38 (d, J=1.8 Hz, 1H), 8.21 (d, J=3.3 Hz, 1H), 8.19 (d, J=4.0 Hz, 1H), 7.59 (dd, J=2.0, 8.9 Hz, 1H), 7.33 (dd, J=5.8, 8.6 Hz, 2H), 7.09 (d, J=9.0 Hz, 1H), 3.87 (s, 2H), 3.01 (s, 3H), 2.59 (s, 3H), 2.48 (s, 3H), 1.34-1.28 (m, 2H), 1.18-1.16 (m, 2H).

### Step 6: 1-[[[3-(1-hydroxyethyl)-6-[6-[(6-methylpyridazin-3-yl)amino]benzimidazol-1-yl]-2-pyridyl]-methyl-amino]methyl]cyclopropanecarbonitrile

Error! Objects cannot be created from editing field codes.

Prepared in analogy to example 53, step 4 using 1-[[[3-acetyl-6-[6-[(6-methylpyridazin-3-yl)amino]benzimidazol-1-yl]-2-pyridyl]-methyl-amino]methyl]cyclopropanecarbonitrile (110.0 mg, 0.240 mmol, 1.0 equiv.) in MeOH:DMF (3:2) to yield 1-[[[3-(1-hydroxyethyl)-6-[6-[(6-methylpyridazin-3-yl)amino]benzimidazol-1-yl]-2-pyridyl]-methyl amino]methyl]cyclopropanecarbonitrile (45.9 mg, 0.100 mmol, 39.1% yield) as light yellow solid. LC-MS: m/z=455.2 [M+H]⁺, ESI pos.

### Example 173

### 1-[[[3-(1-Hydroxyethyl)-6-[5-[(6-methylpyridazin-3-yl)amino]benzimidazol-1-yl]-2-pyridyl]-methyl-amino]methyl]cyclopropanecarbonitrile

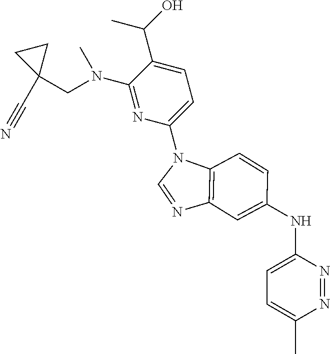 [SMILES: Cc1ccc(Nc2ccc3c(c2)ncn3-c2ccc(C(C)O)c(N(C)CC3(C#N)CC3)n2)nn1]

Prepared in analogy to example 53, step 4 using 1-[[[3-acetyl-6-[5-[(6-methylpyridazin-3-yl)amino]benzimidazol-1-yl]-2-pyridyl]-methyl-amino]methyl]cyclopropanecarbonitrile (110.0 mg, 0.240 mmol, 1.0 equiv., prepared in example 172, step 5) in MeOH:DMF (3:2) to yield 1-[[[3-(1-hydroxyethyl)-6-[5-[(6-methylpyridazin-3-yl)amino]benzimidazol-1-yl]-2-pyridyl]-methyl-amino]methyl]cyclopropanecarbonitrile (53.6 mg, 0.120 mmol, 45.4% yield) as light yellow solid. LC-MS: m/z=455.2 [M+H]⁺, ESI pos.

### Example 174

### 1-[2-(2-Chloro-5-fluoro-3-pyridyl)-6-[5-[(6-methylpyridazin-3-yl)amino]benzimidazol-1-yl]-3-pyridyl]ethanol; formic acid

Error! Objects cannot be created from editing field codes.

### Step 1: 2-chloro-5-fluoro-3-(4,4,5,5-tetramethyl-1,3,2-dioxaborolan-2-yl)pyridine

Error! Objects cannot be created from editing field codes.

Prepared in analogy to example 143, step 2 using 3-bromo-2-chloro-5-fluoro-pyridine (2.0 g, 9.5 mmol, 1.0 equiv.) to give 2-chloro-5-fluoro-3-(4,4,5,5-tetramethyl-1,3,2-dioxaborolan-2-yl)pyridine (2.4 g, 9.22 mmol, 97% yield) as a white solid. LC-MS: m/z=258.2, [M+H]⁺, ESI pos.

### Step 2: 1-[2-(2-chloro-5-fluoro-3-pyridyl)-6-[5-[(6-methylpyridazin-3-yl)amino]benzimidazol-1-yl]-3-pyridyl]ethanone

Error! Objects cannot be created from editing field codes.

Prepared in analogy to example 143, step 2 using 2-chloro-5-fluoro-3-(4,4,5,5-tetramethyl-1,3,2-dioxaborolan-2-yl)pyridine (84.97 mg, 0.330 mmol, 2.5 equiv.) and 1-[2-chloro-6-[5-[(6-methylpyridazin-3-yl)amino]benzimidazol-1-yl]-3-pyridyl]ethanone (50.0 mg, 0.130 mmol, 1.0 equiv., prepared in example 76, step 2) to afford 1-[2-(2-chloro-5-fluoro-3-pyridyl)-6-[5-[(6-methylpyridazin-3-yl)amino]benzimidazol-1-yl]-3-pyridyl]ethanone (25 mg, 0.050 mmol, 39.97% yield) as a yellow solid. LC-MS: m/z=474.3, [M+H]⁺, ESI pos.

### Step 3: 1-[2-(2-chloro-5-fluoro-3-pyridyl)-6-[5-[(6-methylpyridazin-3-yl)amino]benzimidazol-1-yl]-3-pyridyl]ethanol; formic acid

Error! Objects cannot be created from editing field codes.

Prepared in analogy to example 53, step 4 using 1-[2-(2-chloro-5-fluoro-3-pyridyl)-6-[5-[(6-methylpyridazin-3-yl)amino]benzimidazol-1-yl]-3-pyridyl]ethanone and NaBH₄ (6.01 mg, 0.160 mmol, 5 equiv.) to afford 1-[2-(2-chloro-5-fluoro-3-pyridyl)-6-[5-[(6-methylpyridazin-3-yl)amino]benzimidazol-1-yl]-3-pyridyl]ethanol; formic acid (3.2 mg, 0.010 mmol, 21.2% yield) as a yellow solid. LC-MS: m/z=476.3 [M+H]⁺, ESI pos.

### Example 175

### 1-[3-(1-Hydroxyethyl)-6-[6-[[6-[(3-methoxyazetidin-1-yl)methyl]pyridazin-3-yl]amino]benzimidazol-1-yl]-2-pyridyl]-5-methyl-pyrazole-3-carbonitrile; formic acid

Error! Objects cannot be created from editing field codes.

### Step 1: 1-[3-acetyl-6-[5-[[6-[(3-methoxyazetidin-1-yl)methyl]pyridazin-3-yl]amino]benzimidazol-1-yl]-2-pyridyl]-5-methyl-pyrazole-3-carbonitrile and 1-[3-acetyl-6-[6-[[6-[(3-methoxyazetidin-1-yl)methyl]pyridazin-3-yl]amino]benzimidazol-1-yl]-2-pyridyl]-5-methyl-pyrazole-3-carbonitrile

Error! Objects cannot be created from editing field codes.

Prepared in analogy to example 64, step 2 using N-[6-[(3-methoxyazetidin-1-yl)methyl]pyridazin-3-yl]-1H-benzimidazol-5-amine (200.0 mg, 0.640 mmol, 1.0 equiv., prepared in example 141, steps 1 and 2) and 1-(3-acetyl-6-chloro-2-pyridyl)-5-methyl-pyrazole-3-carbonitrile (251.99 mg, 0.970 mmol, 1.5 equiv., prepared in example 64, step 1) to afford 1-[3-acetyl-6-[5-[[6-[(3-methoxyazetidin-1-yl)methyl]pyridazin-3-yl]amino]benzimidazol-1-yl]-2-pyridyl]-5-methyl-pyrazole-3-carbonitrile (40 mg, 0.070 mmol, 11.6% yield) and 1-[3-acetyl-6-[6-[[6-[(3-methoxyazetidin-1-yl)methyl]pyridazin-3-yl]amino]benzimidazol-1-yl]-2-pyridyl]-5-methyl-pyrazole-3-carbonitrile (50 mg, 0.090 mmol, 14.5% yield) as yellow solids. Regioisomer 1: LC-MS: m/z=535.1 [M+H]⁺, ESI pos. ¹H NMR (400 MHz, DMSO-d₆) δ=9.37 (s, 1H), 9.12 (s, 1H), 8.54 (d, J=8.5 Hz, 1H), 8.46 (d, J=2.0 Hz, 1H), 8.30 (d, J=8.5 Hz, 1H), 8.15 (d, J=8.9 Hz, 1H), 7.55 (dd, J=2.0, 9.0 Hz, 1H), 7.40 (d, J=9.1 Hz, 1H), 7.17-7.10 (m, 2H), 3.99 (t, J=5.8 Hz, 1H), 3.75 (s, 2H), 3.55-3.49 (m, 2H), 3.15 (s, 3H), 2.96 (br t, J=6.7 Hz, 2H), 2.54 (s, 3H), 2.20 (s, 3H). Regioisomer 2: ¹H NMR (400 MHz, DMSO-d₆) δ=9.40 (s, 1H), 9.03 (s, 1H), 9.00 (d, J=1.9 Hz, 1H), 8.55 (d, J=8.5 Hz, 1H), 8.28 (d, J=8.5 Hz, 1H), 7.71 (d, J=8.6 Hz, 1H), 7.47 (dd, J=2.1, 8.7 Hz, 1H), 7.37 (d, J=9.1 Hz, 1H), 7.14-7.06 (m, 2H), 4.03-3.95 (m, 1H), 3.75 (s, 2H), 3.55-3.47 (m, 2H), 3.17-3.14 (m, 3H), 2.96 (br dd, J=5.8, 7.9 Hz, 2H), 2.57 (s, 3H), 2.19 (s, 3H).

### Step 2: 1-[3-(-hydroxyethyl)-6-[6-[[6-[(3-methoxyazetidin-1-yl)methyl]pyridazin-3-yl]amino]benzimidazol-1-yl]-2-pyridyl]-5-methyl-pyrazole-3-carbonitrile

Error! Objects cannot be created from editing field codes.

Prepared in analogy to example 53, step 4 using 1-[3-acetyl-6-[5-[[6-[(3-methoxyazetidin-1-yl)methyl]pyridazin-3-yl]amino]benzimidazol-1-yl]-2-pyridyl]-5-methyl-pyrazole-3-carbonitrile (40.0 mg, 0.070 mmol, 1.0 equiv.) in MeOH/DMF (3:1) to afford 1-[3-(1-hydroxyethyl)-6-[6-[[6-[(3-methoxyazetidin-1-yl)methyl]pyridazin-3-yl]amino]benzimidazol-1-yl]-2-pyridyl]-5-methyl-pyrazole-3-carbonitrile; formic acid (20.7 mg, 0.040 mmol, 49.0% yield) as light brown solid. LC-MS: m/z=537.2 [M+H]⁺, ESI pos.

### Example 176

### 1-[3-(1-Hydroxyethyl)-6-[5-[[6-[(3-methoxyazetidin-1-yl)methyl]pyridazin-3-yl]amino]benzimidazol-1-yl]-2-pyridyl]-5-methyl-pyrazole-3-carbonitrile

Error! Objects cannot be created from editing field codes.

Prepared in analogy to example 53, step 4 using 1-[3-acetyl-6-[6-[[6-[(3-methoxyazetidin-1-yl)methyl]pyridazin-3-yl]amino]benzimidazol-1-yl]-2-pyridyl]-5-methyl-pyrazole-3-carbonitrile (50.0 mg, 0.090 mmol, 1.0 equiv., prepared in example 175, step 1) in MeOH/DMF (3:1) to afford 1-[3-(1-hydroxyethyl)-6-[6-[[6-[(3-methoxyazetidin-1-yl)methyl]pyridazin-3-yl]amino]benzimidazol-1-yl]-2-pyridyl]-5-methyl-pyrazole-3-carbonitrile; formic acid (11.9 mg, 0.020 mmol, 23.7% yield) as light yellow solid. LC-MS: m/z=537.2 [M+H]⁺, ESI pos.

### Example 177

### 1-[6-[5-[(6-Methylpyridazin-3-yl)amino]benzimidazol-1-yl]-2-[5-(oxetan-3-yl)-3-(trifluoromethyl)-6,7-dihydro-4H-pyrazolo[4,3-c]pyridin-1-yl]-3-pyridyl]ethanol

Error! Objects cannot be created from editing field codes.

### Step 1: 1-[6-[5-[(6-methylpyridazin-3-yl)amino]benzimidazol-1-yl]-2-[5-(oxetan-3-yl)-3-(trifluoromethyl)-6,7-dihydro-4H-pyrazolo[4,3-c]pyridin-1-yl]-3-pyridyl]ethanone

Error! Objects cannot be created from editing field codes.

Prepared in analogy to example 171, step 4 using 1-[6-[5-[(6-methylpyridazin-3-yl)amino]benzimidazol-1-yl]-2-[3-(trifluoromethyl)-4,5,6,7-tetrahydropyrazolo[4,3-c]pyridin-1-yl]-3-pyridyl]ethanone; hydrochloride (300.0 mg, 0.530 mmol, 1.0 equiv., example 97, step 3) and 3-oxetanone (195.0 mg, 2.71 mmol, 5.14 equiv.) to give 1-[6-[5-[(6-methylpyridazin-3-yl)amino]benzimidazol-1-yl]-2-[5-(oxetan-3-yl)-3-(trifluoromethyl)-6,7-dihydro-4H-pyrazolo[4,3-c]pyridin-1-yl]-3-pyridyl]ethanone (150 mg, 0.250 mmol, 33.84% yield) as light yellow solid. LC-MS: m/z=590.2 [M+H]⁺, ESI pos.

### Step 2: 1-[6-[5-[(6-methylpyridazin-3-yl)amino]benzimidazol-1-yl]-2-[5-(oxetan-3-yl)-3-(trifluoromethyl)-6,7-dihydro-4H-pyrazolo[4,3-c]pyridin-1-yl]-3-pyridyl]ethanol

Error! Objects cannot be created from editing field codes.

Prepared in analogy to example 53, step 4 using 1-[6-[5-[(6-methylpyridazin-3-yl)amino]benzimidazol-1-yl]-2-[5-(oxetan-3-yl)-3-(trifluoromethyl)-6,7-dihydro-4H-pyrazolo[4,3-c]pyridin-1-yl]-3-pyridyl]ethanone (150.0 mg, 0.180 mmol, 1.0 equiv.) and NaBH₄ (30.0 mg, 0.790 mmol, 4.5 equiv.) in MeOH/DCM (1:9) to afford 1-[6-[5-[(6-methylpyridazin-3-yl)amino]benzimidazol-1-yl]-2-[5-(oxetan-3-yl)-3-(trifluoromethyl)-6,7-dihydro-4H-pyrazolo[4,3-c]pyridin-1-yl]-3-pyridyl]ethanol (45.2 mg, 0.080 mmol, 42.9% yield) as light yellow solid. LC-MS:m/z=592.3 [M+H]⁺, ESI pos.

### Example 178

### 1-[3-(1-Hydroxyethyl)-6-[6-[(6-methylpyridazin-3-yl)amino]benzimidazol-1-yl]-2-pyridyl]-3-methyl-pyrazole-4-carbonitrile

Error! Objects cannot be created from editing field codes.

Prepared in analogy to example 53, step 4 using 1-[3-acetyl-6-[6-[(6-methylpyridazin-3-yl)amino]benzimidazol-1-yl]-2-pyridyl]-3-methyl-pyrazole-4-carbonitrile (85.0 mg, 0.190 mmol, 1.0 equiv., prepared in example 150, step 2) in MeOH/THF/DCM (1:1:1) to afford 1-[3-(1-hydroxyethyl)-6-[6-[(6-methylpyridazin-3-yl)amino]benzimidazol-1-yl]-2-pyridyl]-3-methyl-pyrazole-4-carbonitrile (51.8 mg, 0.110 mmol, 60.49% yield) as off-white solid. LC-MS: m/z=452.1 [M+H]⁺, ESI pos.

### Example 179

### 1-[2-[2-(Methylamino)-3-pyridyl]-6-[5-[(6-methylpyridazin-3-yl)amino]benzimidazol-1-yl]-3-pyridyl]ethanol; formic acid

### Step 1: [2-(methylamino)-3-pyridyl]boronic acid

Error! Objects cannot be created from editing field codes.

Prepared in analogy to example 143, step 2 using 3-bromo-N-methyl-pyridin-2-amine (1.0 g, 5.35 mmol, 1.0 equiv.) to afford [2-(methylamino)-3-pyridyl]boronic acid (721 mg, 4.69 mmol, 87% yield) as a white solid. LC-MS: m/z=153.2, [M+H]⁺, ESI pos.

### Step 2:1-[2-[2-(methylamino)-3-pyridyl]-6-[5-[(6-methylpyridazin-3-yl)amino]benzimidazol-1-yl]-3-pyridyl]ethanol; formic acid

Error! Objects cannot be created from editing field codes.

Prepared in analogy to example 163, Step 3 using [2-(methylamino)-3-pyridyl]boronic acid (159.61 mg, 1.05 mmol, 8.0 equiv.) (used without purification) and 1-[2-chloro-6-[5-[(6-methylpyridazin-3-yl)amino]benzimidazol-1-yl]-3-pyridyl]ethanol (50.0 mg, 0.130 mmol, 1.0 equiv, prepared in example 163, step 2) to afford 1-[2-[2-(methylamino)-3-pyridyl]-6-[5-[(6-methylpyridazin-3-yl)amino]benzimidazol-1-yl]-3-pyridyl]ethanol; formic acid (17.8 mg, 0.040 mmol, 27.26% yield) as a yellow solid. LC-MS: 453.2, [M+H]⁺, ESI pos.

### Example 182

### 1-[3-(1-Hydroxyethyl)-6-[5-[(6-methylpyridazin-3-yl)amino]benzimidazol-1-yl]-2-pyridyl]-3-methyl-pyrazole-4-carbonitrile

Error! Objects cannot be created from editing field codes.

Prepared in analogy to example 53, step 4 using 1-[3-acetyl-6-[5-[(6-methylpyridazin-3-yl)amino]benzimidazol-1-yl]-2-pyridyl]-3-methyl-pyrazole-4-carbonitrile (45.0 mg, 0.100 mmol, 1.0 equiv., prepared in example 178, step 2) in MeOH/THF/DCM (1:1:1) to afford 1-[3-(1-hydroxyethyl)-6-[5-[(6-methylpyridazin-3-yl)amino]benzimidazol-1-yl]-2-pyridyl]-3-methyl-pyrazole-4-carbonitrile (9.8 mg, 0.020 mmol, 21.3% yield) as off-white solid. LC-MS: m/z=452.1 [M+H]⁺, ESI pos. ¹H NMR (400 MHz, CD₃OD) δ=9.06-9.01 (m, 1H), 8.98-8.92 (m, 1H), 8.55-8.49 (m, 1H), 8.26-8.22 (m, 1H), 8.20-8.15 (m, 1H), 8.05-7.99 (m, 1H), 7.70-7.65 (m, 1H), 7.43-7.37 (m, 1H), 7.21-7.13 (m, 1H), 5.61-5.51 (m, 1H), 2.56 (s, 3H), 2.52 (s, 3H), 1.52 (d, J=6.4 Hz, 3H).

### Example 183

### 1-[3-(1-Hydroxyethyl)-6-[5-[(6-methylpyridazin-3-yl)amino]benzimidazol-1-yl]-2-pyridyl]-3-methyl-azetidine-3-carbonitrile

Error! Objects cannot be created from editing field codes.

### Step 1: 1-(3-acetyl-6-chloro-2-pyridyl)-3-methyl-azetidine-3-carbonitrile

Error! Objects cannot be created from editing field codes.

Prepared in analogy to example 62, step 1 using 3-methyl-azetidine-3-carbonitrile (49.7 mg, 0.374 mmol, 1.0 equiv.) and 1-(6-chloro-2-fluoro-3-pyridyl)ethanone (CAS #1260663-13-5, 65 mg, 0.374 mmol, 1.0 equiv.) yielding the title compound (75 mg, 80.2% yield) as a light brown solid. LC-MS: m/z=254.16 [M+H]⁺, ESI pos.

### Step 2: 1-[3-acetyl-6-[6-[(6-methylpyridazin-3-yl)amino]benzimidazol-1-yl]-2-pyridyl]-3-methyl-azetidine-3-carbonitrile and 1-[3-acetyl-6-[5-[(6-methylpyridazin-3-yl)amino]benzimidazol-1-yl]-2-pyridyl]-3-methyl-azetidine-3-carbonitrile

Error! Objects cannot be created from editing field codes.

Prepared in analogy to example 64, step 2 using 1-(3-acetyl-6-chloro-2-pyridyl)-3-methyl-azetidine-3-carbonitrile (72 mg, 0.288 mmol, 1.0 equiv.) and 1H-benzimidazol-5-yl-(6-methylpyridazin-3-yl)amine (64.95 mg, 0.288 mmol, 1.0 equiv., prepared in example 64, intermediate 1) to afford: 1-[3-acetyl-6-[6-[(6-methylpyridazin-3-yl)amino]benzimidazol-1-yl]-2-pyridyl]-3-methyl-azetidine-3-carbonitrile (30.8 mg, 24.4% yield) and 1-[3-acetyl-6-[5-[(6-methylpyridazin-3-yl)amino]benzimidazol-1-yl]-2-pyridyl]-3-methyl-azetidine-3-carbonitrile (22.5 mg, 17.8% yield) after separation by SFC (Chiral OJ 250 mm×20 mm×10 μm, 25% MeOH (with 0.2% HNEt₂), 90 mL/min flow rate). Regioisomer 1: LC-MS: m/z=423.2 (M+H)+, ESI pos. ¹H NMR (600 MHz, DMSO-d₆) δ=9.22 (s, 1H), 9.03 (s, 1H), 8.35 (d, J=8.5 Hz, 1H), 8.32 (d, J=2.1 Hz, 1H), 8.29 (d, J=8.8 Hz, 1H), 7.61 (dd, J=8.9, 2.1 Hz, 1H), 7.35 (dd, J=8.7, 7.5 Hz, 2H), 7.11 (d, J=9.1 Hz, 1H), 4.41 (d, J=9.4 Hz, 2H), 4.05 (d, J=9.4 Hz, 2H), 2.57 (s, 3H), 2.49 (s, 3H), 1.67 (s, 3H). Regioisomer 2: LC-MS: m/z=423.2 (M+H)+, ESI pos. ¹H NMR (600 MHz, DMSO-d₆) δ=9.60 (d, J=1.5 Hz, 1H), 9.28-9.28 (m, 1H), 9.30 (s, 1H), 8.96 (s, 1H), 8.34 (d, J=8.4 Hz, 1H), 7.65 (d, J=8.7 Hz, 1H), 7.32-7.39 (m, 2H), 7.18 (dd, J=8.7, 2.2 Hz, 1H), 7.12 (d, J=9.0 Hz, 1H), 4.53 (br d, J=9.9 Hz, 2H), 4.17 (br d, J=9.8 Hz, 2H), 2.57 (s, 3H), 2.53 (s, 3H), 2.40-2.40 (m, 1H), 1.70 (s, 3H).

### Step 3: 1-[3-(-hydroxyethyl)-6-[5-[(6-methylpyridazin-3-yl)amino]benzimidazol-1-yl]-2-pyridyl]-3-methyl-azetidine-3-carbonitrile

Error! Objects cannot be created from editing field codes.

Prepared in analogy to example 53, step 4 using 1-[3-acetyl-6-[5-[(6-methylpyridazin-3-yl)amino]benzimidazol-1-yl]-2-pyridyl]-3-methyl-azetidine-3-carbonitrile (22.5 mg, 0.051 mmol, 1.0 equiv.) and NaBH₄ (2.91 mg, 0.077 mmol, 1.5 equiv.) in THF/ⁱPrOH (1:1) to obtain 1-[3-(1-hydroxyethyl)-6-[5-[(6-methylpyridazin-3-yl)amino]benzimidazol-1-yl]-2-pyridyl]-3-methyl-azetidine-3-carbonitrile (17 mg, 72.53%) as a white solid. LC-MS: m/z=441.21 [M+H]⁺, ESI pos.

### Example 184

### 1-[3-(1-Hydroxyethyl)-6-[6-[(6-methylpyridazin-3-yl)amino]benzimidazol-1-yl]-2-pyridyl]-3-methyl-azetidine-3-carbonitrile

Error! Objects cannot be created from editing field codes.

Prepared in analogy to example 53, step 4 using 1-[3-acetyl-6-[6-[(6-methylpyridazin-3-yl)amino]benzimidazol-1-yl]-2-pyridyl]-3-methyl-azetidine-3-carbonitrile (30.5 mg, 0.070 mmol, 1.0 equiv) and NaBH₄ (3.95 mg, 0.104 mmol, 1.5 equiv.) in THF/ⁱPrOH (1:1) to obtain 1-[3-(1-hydroxyethyl)-6-[6-[(6-methylpyridazin-3-yl)amino]benzimidazol-1-yl]-2-pyridyl]-3-methyl-azetidine-3-carbonitrile (13.5 mg, 42.61%) as white solid. LC-MS: m/z=441.21 [M+H]⁺, ESI pos.

### Example 185

### 1-[3-(1-Hydroxyethyl)-6-[6-methoxy-5-[(6-methylpyridazin-3-yl)amino]benzimidazol-1-yl]-2-pyridyl]-5-methyl-pyrazole-3-carbonitrile

Error! Objects cannot be created from editing field codes.

### Step 1: 1-[3-acetyl-6-(5-bromo-6-methoxy-benzimidazol-1-yl)-2-pyridyl]-5-methyl-pyrazole-3-carbonitrile; formic acid

Error! Objects cannot be created from editing field codes.

Prepared in analogy to example 64, step 2 using 1-(3-acetyl-6-chloro-2-pyridyl)-5-methyl-pyrazole-3-carbonitrile (1.0 g, 3.84 mmol, 1.0 equiv., prepared in example 64, step 1) and 5-bromo-6-methoxy-1H-benzimidazole (871.03 mg, 3.84 mmol, 1.0 equiv.) and potassium carbonate (1.06 g, 7.67 mmol, 2.0 equiv.) to give 1-[3-acetyl-6-(5-bromo-6-methoxy-benzimidazol-1-yl)-2-pyridyl]-5-methyl-pyrazole-3-carbonitrile; formic acid (409.5 mg, 0.820 mmol, 20.4% yield) as orange solid. LC-MS: m/z 452.9 [M+H]⁺, ESI pos.

### Step 2: 1-[3-acetyl-6-[6-methoxy-5-[(6-methylpyridazin-3-yl)amino]benzimidazol-1-yl]-2-pyridyl]-5-methyl-pyrazole-3-carbonitrile

Error! Objects cannot be created from editing field codes.

Prepared in analogy to example 104, step 2 using 1-[3-acetyl-6-(5-bromo-6-methoxy-benzimidazol-1-yl)-2-pyridyl]-5-methyl-pyrazole-3-carbonitrile; formic acid (50 mg, 0.101 mmol, 1.0 equiv. and, (6-methylpyridazin-3-yl)amine (21.94 mg, 0.201 mmol, 2.0 equiv.) to afford the title compound (30.4 mg, 61.8% yield). LC-MS: m/z=480.3 [M+H]⁺, ESI pos.

### Step 3: 1-[3-(1-hydroxyethyl)-6-[6-methoxy-5-[(6-methylpyridazin-3-yl)amino]benzimidazol-1-yl]-2-pyridyl]-5-methyl-pyrazole-3-carbonitrile

Error! Objects cannot be created from editing field codes.

Prepared in analogy to example 53, step 4 using 1-[3-acetyl-6-[6-methoxy-5-[(6-methylpyridazin-3-yl)amino]benzimidazol-1-yl]-2-pyridyl]-5-methyl-pyrazole-3-carbonitrile (30.4 mg, 0.062 mmol, 1.0 equiv.) in MeOH/THF (1:1) to afford the title compound (21.6 mg, 71.5% yield). LC-MS: m/z=480.3 [M+H]⁺, ESI pos.

### Example 186

### 1-[6-[5-[(6-Methylpyridazin-3-yl)amino]benzimidazol-1-yl]-2-[2-(1H-pyrazol-4-yl)-3-pyridyl]-3-pyridyl]ethanol

Error! Objects cannot be created from editing field codes.

### Step 1: 1-[2-[2-[1-[(4-methoxyphenyl)methyl]pyrazol-4-yl]-3-pyridyl]-6-[5-[(6-methylpyridazin-3-yl)amino]benzimidazol-1-yl]-3-pyridyl]ethanone

Error! Objects cannot be created from editing field codes.

Prepared in analogy to example 143, step 2 using 1-[2-chloro-6-[5-[(6-methylpyridazin-3-yl)amino]benzimidazol-1-yl]-3-pyridyl]ethanone (70.0 mg, 0.180 mmol, 1.0 equiv., prepared in example 76, step 2), and [2-[1-[(4-methoxyphenyl)methyl]pyrazol-4-yl]-3-pyridyl]boronic acid (114.24 mg, 0.370 mmol, 2 equiv.) to afford 1-[2-[2-[1-[(4-methoxyphenyl)methyl]pyrazol-4-yl]-3-pyridyl]-6-[5-[(6-methylpyridazin-3-yl)amino]benzimidazol-1-yl]-3-pyridyl]ethanone (50 mg, 0.080 mmol, 44.5% yield) as yellow solid. LC-MS: m/z=608.2 [M+H]⁺, ESI pos.

### Step 2: 1-[6-[5-[(6-methylpyridazin-3-yl)amino]benzimidazol-1-yl]-2-[2-(H-pyrazol-4-yl)-3-pyridyl]-3-pyridyl]ethanone, formic acid

Error! Objects cannot be created from editing field codes.

A mixture of 1-[2-[2-[1-[(4-methoxyphenyl)methyl]pyrazol-4-yl]-3-pyridyl]-6-[5-[(6-methylpyridazin-3-yl)amino]benzimidazol-1-yl]-3-pyridyl]ethanone (45.0 mg, 0.070 mmol, 1.0 equiv.) and 1 mL TFA was stirred at 60° C. for 4 hours. The reaction mixture was concentrated under reduced pressure and the residue was purified by HPLC (Phenomenex Luna C18 (150 mm×40 mm×15 μm). Flow rate: 60 mL/min. Gradient: 10% to 40% CH₃CN in (0.225% formic acid in H₂O v/v) (13 min) then 100% CH₃CN (2 min)) and lyophilized to give 1-[6-[5-[(6-methylpyridazin-3-yl)amino]benzimidazol-1-yl]-2-[2-(1H-pyrazol-4-yl)-3-pyridyl]-3-pyridyl]ethanone (30 mg, 0.060 mmol, 83.1% yield) as yellow solid. LC-MS: m/z=488.1 [M+H]⁺, ESI pos.

### Step 3: 1-[6-[5-[(6-methylpyridazin-3-yl)amino]benzimidazol-1-yl]-2-[2-(H-pyrazol-4-yl)-3-pyridyl]-3-pyridyl]ethanol

Error! Objects cannot be created from editing field codes.

Prepared in analogy to example 53, step 4 using 1-[6-[5-[(6-methylpyridazin-3-yl)amino]benzimidazol-1-yl]-2-[2-(1H-pyrazol-4-yl)-3-pyridyl]-3-pyridyl]ethanone (25.0 mg, 0.050 mmol, 1.0 equiv.) and NaBH₄ (9.75 mg, 0.260 mmol, 5 equiv.) to yield 1-[6-[5-[(6-methylpyridazin-3-yl)amino]benzimidazol-1-yl]-2-[2-(1H-pyrazol-4-yl)-3-pyridyl]-3-pyridyl]ethanol (10.6 mg, 0.020 mmol, 41.5% yield) as a white solid. LC-MS: m/z=490.2 [M+H]⁺, ESI pos.

### Example 187

### 1-[3-(1-Hydroxyethyl)-6-[6-(2-methoxyethoxy)-5-[(6-methylpyridazin-3-yl)amino]benzimidazol-1-yl]-2-pyridyl]-5-methyl-pyrazole-3-carbonitrile

Error! Objects cannot be created from editing field codes.

### Step 1: 5-bromo-6-(2-methoxyethoxy)-1H-benzimidazole

A solution of 4-bromo-5-(2-methoxyethoxy)benzene-1,2-diamine (1.6 g, 6.13 mmol, 1.0 equiv.) in trimethyl orthoformate (50.0 mL, 457.03 mmol, 74.59 equiv.) was stirred at 120° C. for 3 hours. The mixture was concentrated in vacuo and the residue was dissolved in THF and 1N aq. HCl. The resulting mixture was stirred at 25° C. for 1 hour. The pH was then adjusted to 8 with NaHCO₃, and extracted with EtOAc. The combined organic layers were dried over Na₂SO₄, the volatiles evaporated and the residue was purified by preparative HPLC (Waters Xbridge C18 150 mm×50 mm×10 μm, gradient 14-4% CH₃CN in H₂O (with 10 mM NH₄HCO₃) over 11 min, then 100% CH₃CN (with 10 mM NH₄HCO₃) (2 min), flow rate 60 mL/min) to yield 5-bromo-6-(2-methoxyethoxy)-1H-benzimidazole (470 mg, 1.73 mmol, 28.29% yield) as yellow solid. LC-MS: m/z=271.0 [M+H]⁺, ESI pos.

### Step 2: 1-[3-acetyl-6-[5-bromo-6-(2-methoxyethoxy)benzimidazol-1-yl]-2-pyridyl]-5-methyl-pyrazole-3-carbonitrile

Error! Objects cannot be created from editing field codes.

Prepared in analogy to example 64, step 2 using 5-bromo-6-(2-methoxyethoxy)-1H-benzimidazole (460.0 mg, 1.7 mmol, 1.0 equiv.) and 1-(3-acetyl-6-chloro-2-pyridyl)-5-methyl-pyrazole-3-carbonitrile (574.99 mg, 2.21 mmol, 1.3 equiv., prepared in example 64, step 1) to afford 1-[3-acetyl-6-[5-bromo-6-(2-methoxyethoxy)benzimidazol-1-yl]-2-pyridyl]-5-methyl-pyrazole-3-carbonitrile (221.5 mg, 0.450 mmol, 23.72% yield) as brown solid. LC-MS: m/z=495.0 [M+H]⁺, ESI pos.

### Step 3: 1-[3-acetyl-6-[6-(2-methoxyethoxy)-5-[(6-methylpyridazin-3-yl)amino]benzimidazol-1-yl]-2-pyridyl]-5-methyl-pyrazole-3-carbonitrile

Error! Objects cannot be created from editing field codes.

Prepared in analogy to example 104, step 2, using 1-[3-acetyl-6-[5-bromo-6-(2-methoxyethoxy)benzimidazol-1-yl]-2-pyridyl]-5-methyl-pyrazole-3-carbonitrile (50 mg, 0.101 mmol, 1 equiv.), and (6-methylpyridazin-3-yl)amine (22.03 mg, 0.202 mmol, 2.0 equiv., prepared in example 64, intermediate 1) to afford the title compound (35.5 mg, 63.8% yield) as yellow solid. LC-MS: m/z=524.3 [M+H]⁺, ESI pos.

### Step 4: 1-[3-(1-hydroxyethyl)-6-[6-(2-methoxyethoxy)-5-[(6-methylpyridazin-3-yl)amino]benzimidazol-1-yl]-2-pyridyl]-5-methyl-pyrazole-3-carbonitrile

Error! Objects cannot be created from editing field codes.

Prepared in analogy to example 53, step 4 using 1-[3-acetyl-6-[6-(2-methoxyethoxy)-5-[(6-methylpyridazin-3-yl)amino]benzimidazol-1-yl]-2-pyridyl]-5-methyl-pyrazole-3-carbonitrile (35.5 mg, 0.064 mmol, 1.0 equiv.) in MeOH/THF (1:1) to afford the title compound (23.2 mg, 67.2% yield) as yellow solid. LC-MS: m/z=526.3 [M+H]⁺, ESI pos.

### Example 188

### 1-[6-[5-fluoro-6-[[(3R,4S)-4-fluoropyrrolidin-3-yl]amino]benzimidazol-1-yl]-3-(1-hydroxyethyl)pyridin-2-yl]-5-methylpyrazole-3-carbonitrile

Error! Objects cannot be created from editing field codes.
Error! Objects cannot be created from editing field codes.

### Step 1: 1-[3-acetyl-6-[5-fluoro-6-[[(3S,4R)-4-fluoropyrrolidin-3-yl]amino]benzimidazol-1-yl]-2-pyridyl]-5-methyl-pyrazole-3-carbonitrile; 2,2,2-trifluoroacetic acid

Error! Objects cannot be created from editing field codes.

A solution of tert-butyl (3S,4R)-3-[[3-[5-acetyl-6-(3-cyano-5-methyl-pyrazol-1-yl)-2-pyridyl]-6-fluoro-benzimidazol-5-yl]amino]-4-fluoro-pyrrolidine-1-carboxylate (40.0 mg, 0.070 mmol, 1.0 equiv., prepared in example 166, step 4) and 0.5 mL TFA in DCM (2.5 mL) was stirred at 25° C. for 1 hour. The mixture was concentrated in vacuo to give crude 1-[3-acetyl-6-[5-fluoro-6-[[(3S,4R)-4-fluoropyrrolidin-3-yl]amino]benzimidazol-1-yl]-2-pyridyl]-5-methyl-pyrazole-3-carbonitrile; 2,2,2-trifluoroacetic acid which was used into next step without further purification. LC-MS: m/z=463.1 [M+H]⁺, ESI pos.

### Step 2: 1-[6-[5fluoro-6-[[(3S,4R)-4-fluoropyrrolidin-3-yl]amino]benzimidazol-1-yl]-3-(1-hydroxyethyl)-2-pyridyl]-5-methyl-pyrazole-3-carbonitrile

Error! Objects cannot be created from editing field codes.

Prepared in analogy to example 53, step 4 using 1-[3-acetyl-6-[5-fluoro-6-[[(3S,4R)-4-fluoropyrrolidin-3-yl]amino]benzimidazol-1-yl]-2-pyridyl]-5-methyl-pyrazole-3-carbonitrile; 2,2,2-trifluoroacetic acid (as crude from the previous step) to yield 1-[6-[5-fluoro-6-[[(3S,4R)-4-fluoropyrrolidin-3-yl]amino]benzimidazol-1-yl]-3-(1-hydroxyethyl)-2-pyridyl]-5-methyl-pyrazole-3-carbonitrile (19.1 mg, 0.040 mmol, 37.5% yield) as white solid. LC-MS: m/z=465.2 [M+H]⁺, ESI pos. ¹H NMR (400 MHz, DMSO-d₆) δ=8.88-8.77 (m, 1H), 8.44 (d, J=8.5 Hz, 1H), 8.28 (s, 1H), 8.24 (dd, J=1.9, 8.5 Hz, 1H), 7.55 (dd, J=4.6, 8.1 Hz, 1H), 7.50 (d, J=11.8 Hz, 1H), 7.12-6.98 (m, 1H), 5.36 (d, J=6.9 Hz, 1H), 5.06-4.81 (m, 1H), 4.65-4.52 (m, 1H), 3.24-2.96 (m, 4H), 2.84 (t, J=9.9 Hz, 1H), 2.33 (d, J=1.0 Hz, 3H), 2.07 (s, 1H), 1.27 (dd, J=6.4, 12.0 Hz, 3H).

### Example 189

### 1-[6-[6-[2-(Dimethylamino)ethoxy]-5-[(6-methylpyridazin-3-yl)amino]benzimidazol-1-yl]-3-(1-hydroxyethyl)-2-pyridyl]-5-methyl-pyrazole-3-carbonitrile

Error! Objects cannot be created from editing field codes.

### Step 1: 2-(4,5-diamino-2-bromo-phenoxy)ethyl-dimethyl-amine

Error! Objects cannot be created from editing field codes.

Under an argon atmosphere, 2-(5-amino-2-bromo-4-nitro-phenoxy)ethyl-dimethyl-amine (4.52 g, 13.38 mmol, 1.0 equiv.) was dissolved in ethanol (190 mL). Activated zinc (8.74 g, 133.75 mmol, 10.0 equiv.) was added and the reaction mixture was cooled to 0° C. A solution containing acetic acid (5.62 g, 5.36 mL, 93.63 mmol, 7.0 equiv.) in ethanol (49.45 mL) was added dropwise while the temperature was kept below 5° C. The cooling bath was removed, and the reaction mixture was allowed to warm to RT while stirring for another 2 hours. The reaction mixture was filtered, the filter cake rinsed with EtOH, and the combined filtrates evaporated to dryness. The residue was dissolved in Na₂CO₃ (2N) and extracted with DCM. The organic layer was washed with brine, dried over MgSO₄ and the volatiles evaporated. The crude product was purified over SiNH₂ (50 g) using a gradient from 0-5% MeOH in DCM to afford the title compound (3.24 g, 83.9% yield) as purple liquid. LC-MS: m/z=276.1 [M+H]⁺, ESI pos.

### Step 2: 2-[(6-bromo-3H-benzimidazol-5-yl)oxy]ethyl-dimethyl-amine

Error! Objects cannot be created from editing field codes.

A solution of 2-(4,5-diamino-2-bromo-phenoxy)ethyl-dimethyl-amine (3.24 g, 11.82 mmol, 1.0 equiv.) in trimethyl orthoformate (93.56 g, 96.45 mL, 881.62 mmol, 75 equiv.) was stirred at 120° C. for 3 hours. The reaction mixture was evaporated to dryness. The crude product was dissolved in DCM and extracted with aq. HCl (1N). The aq. layer was cooled, and 4N NaOH solution was added until the solution was basic. The product was extracted with DCM. The organic layer was washed with brine, dried over MgSO₄, and the volatiles removed. The crude product was purified by flash column chromatography (SiNH₂, 0-5% MeOH in DCM) to yield the title compound (984 mg, 27.8% yield) as light yellow foam. LC-MS: m/z=285.1 [M+H]⁺, ESI pos.

### Step 3: 1-[3-acetyl-6-[5-bromo-6-[2-(dimethylamino)ethoxy]benzimidazol-1-yl]-2-pyridyl]-5-methyl-pyrazole-3-carbonitrile

Error! Objects cannot be created from editing field codes.

Prepared in analogy to example 65, step 1 using 1-(3-acetyl-6-chloro-2-pyridyl)-5-methyl-pyrazole-3-carbonitrile (166.05 mg, 0.637 mmol, 1.0 equiv.) and 2-[(6-bromo-3H-benzimidazol-5-yl)oxy]ethyl-dimethyl-amine (181. mg, 0.637 mmol, 1.0 equiv.) to afford the title compound (62.3 mg, 17.3% yield) as orange solid. LC-MS: m/z=510.1 [M+H]⁺, ESI pos.

### Step 4: 1-[3-acetyl-6-[6-[2-(dimethylamino)ethoxy]-5-[(6-methylpyridazin-3-yl)amino]benzimidazol-1-yl]-2-pyridyl]-5-methyl-pyrazole-3-carbonitrile

Error! Objects cannot be created from editing field codes.

Prepared according Example 65, step 2 using 1-[3-acetyl-6-[5-bromo-6-[2-(dimethylamino)ethoxy]benzimidazol-1-yl]-2-pyridyl]-5-methyl-pyrazole-3-carbonitrile (41.1 mg, 0.081 mmol, 1.0 equiv.), (6-methylpyridazin-3-yl)amine (17.65 mg, 0.162 mmol, 2.0 equiv.) to afford the title compound (20.5 mg, 44.9% yield) as yellow solid. LC-MS: m/z=537.3 [M+H]⁺, ESI pos.

### Step 5: 1-[6-[6-[2-(dimethylamino)ethoxy]-5-[(6-methylpyridazin-3-yl)amino]benzimidazol-1-yl]-3-(1-hydroxyethyl)-2-pyridyl]-5-methyl-pyrazole-3-carbonitrile

Error! Objects cannot be created from editing field codes.

Prepared according to example 52, step 4 using 1-[3-acetyl-6-[6-[2-(dimethylamino)ethoxy]-5-[(6-methylpyridazin-3-yl)amino]benzimidazol-1-yl]-2-pyridyl]-5-methyl-pyrazole-3-carbonitrile (20.5 mg, 0.038 mmol, 1 equiv.) in MeOH/THF (1:1) to yield the title compound (13.3 mg, 63.3% yield) as light yellow solid. LC-MS: m/z=539.3 [M+H]⁺, ESI pos.

### Example 190

### 1-[6-[5-Bromo-6-[4-(oxetan-3-yl)piperazino]benzimidazol-1-yl]-3-(1-hydroxyethyl)-2-pyridyl]-5-methyl-pyrazole-3-carbonitrile

Error! Objects cannot be created from editing field codes.

Prepared according to example 53, step 4 using 1-[3-acetyl-6-[5-bromo-6-[4-(oxetan-3-yl)piperazin-1-yl]benzimidazol-1-yl]-2-pyridyl]-5-methyl-pyrazole-3-carbonitrile (30 mg, 0.053 mmol, 1.0 equiv., prepared in example 130, step 4) in MeOH/THF (1:1). LC-MS: m/z=565.2 [M+H]⁺, ESI pos.

### Example 191

### 1-[3-(1-Hydroxyethyl)-6-[5-[[6-(3-methoxyazetidine-1-carbonyl)pyridazin-3-yl]amino]benzimidazol-1-yl]-2-pyridyl]-5-methyl-pyrazole-3-carbonitrile

Error! Objects cannot be created from editing field codes.

### Step 1: (6-aminopyridazin-3-yl)-(3-methoxyazetidin-1-yl)methanone; 2,2,2-trifluoroacetic acid

Error! Objects cannot be created from editing field codes.

Under argon atmosphere, N-[6-(3-methoxyazetidine-1-carbonyl)pyridazin-3-yl]carbamic acid tert-butyl ester (88 mg, 0.285 mmol, 1.0 equiv.) was dissolved in DCM (1 mL). TFA (813.6 mg, 550 μL, 7.14 mmol, 25 equiv.) was added, and the reaction mixture was stirred at RT over 1.5 hours. The volatiles were evaporated, and the crude title compound as dark brown oil was used without further purification. LC-MS: m/z=209.1 [M+H]⁺, ESI pos.

### Step 2: 1-[3-acetyl-6-[5-[[6-(3-methoxyazetidine-1-carbonyl)pyridazin-3-yl]amino]benzimidazol-1-yl]-2-pyridyl]-5-methyl-pyrazole-3-carbonitrile

Error! Objects cannot be created from editing field codes.

Prepared in analogy to example 104, step 2 using 1-[3-acetyl-6-(5-bromobenzimidazol-1-yl)-2-pyridyl]-5-methyl-pyrazole-3-carbonitrile (60 mg, 0.142 mmol, 1.0 equiv., prepared in example 65, step 1) and (6-aminopyridazin-3-yl)-(3-methoxyazetidin-1-yl)methanone; 2,2,2-trifluoroacetic acid (used as crude from the previous step) to afford the title compound (28 mg, 10.8% yield, 30% purity) as yellow oil. LC-MS: m/z=549.2 [M+H]⁺, ESI pos.

### Step 3: 1-[3-(1-hydroxyethyl)-6-[5-[[6-(3-methoxyazetidine-1-carbonyl)pyridazin-3-yl]amino]benzimidazol-1-yl]-2-pyridyl]-5-methyl-pyrazole-3-carbonitrile

Error! Objects cannot be created from editing field codes.

Prepared in analogy to example 53, step 4 using 1-[3-acetyl-6-[5-[[6-(3-methoxyazetidine-1-carbonyl)pyridazin-3-yl]amino]benzimidazol-1-yl]-2-pyridyl]-5-methyl-pyrazole-3-carbonitrile (28 mg, 0.051 mmol, 1.0 equiv.) to yield the title compound (1.0 mg, 3.6% yield) as white lyophilized powder. LC-MS: m/z=515.3 [M+H]⁺, ESI pos.

### Example 192

### 1-[3-(1-Hydroxyethyl)-6-[5-[(6-methylpyridazin-3-yl)amino]-6-pyrrolidin-3-yloxy-benzimidazol-1-yl]-2-pyridyl]-5-methyl-pyrazole-3-carbonitrile

Error! Objects cannot be created from editing field codes.

### Step 1: tert-butyl 3-[(6-bromo-3H-benzimidazol-5-yl)oxy]pyrrolidine-1-carboxylate

Error! Objects cannot be created from editing field codes.

A mixture of tert-butyl 3-(4,5-diamino-2-bromo-phenoxy)pyrrolidine-1-carboxylate (520.0 mg, 1.4 mmol, 1.0 equiv.) in trimethyl orthoformate (30.0 mL, 274.22 mmol, 196.3 equiv.) was heated to 120° C. for 12 hours. The mixture was concentrated, purified by reversed phase (FA) and lyophilizated to give tert-butyl 3-[(6-bromo-3H-benzimidazol-5-yl)oxy]pyrrolidine-1-carboxylate (447 mg, 1.17 mmol, 78.5% yield) as a red oil. LC-MS: m/z=326.1 [M-56+H]⁺, ESI pos.

### Step 2: tert-butyl 3-[3-[5-acetyl-6-(3-cyano-5-methyl-pyrazol-1-yl)-2-pyridyl]-6-bromo-benzimidazol-5-yl]oxypyrrolidine-1-carboxylate

Error! Objects cannot be created from editing field codes.

Prepared in analogy to example 64, step 2 using tert-butyl 3-[(6-bromo-3H-benzimidazol-5-yl)oxy]pyrrolidine-1-carboxylate (397.0 mg, 1.04 mmol, 1.0 equiv.) and 1-(3-acetyl-6-chloro-2-pyridyl)-5-methyl-pyrazole-3-carbonitrile (270.74 mg, 1.04 mmol, 1.0 equiv., prepared in example 64, step 1) to afford tert-butyl 3-[3-[5-acetyl-6-(3-cyano-5-methyl-pyrazol-1-yl)-2-pyridyl]-6-bromo-benzimidazol-5-yl]oxypyrrolidine-1-carboxylate (131.5 mg, 0.220 mmol, 20.0% yield) as an off-white solid. LC-MS: m/z=605.9 [M+H]⁺, ESI pos. ¹H NMR (400 MHz, CD₃OD) δ=8.94 (s, 1H), 8.53 (br d, J=8.0 Hz, 1H), 8.16 (br d, J=8.0 Hz, 1H), 7.98 (s, 2H), 6.91 (s, 1H), 5.01 (br s, 1H), 3.61-3.51 (m, 4H), 2.53 (s, 3H), 2.27 (s, 3H), 1.47 (br d, J=16.0 Hz, 9H)

### Step 3: 3-[3-[5-acetyl-6-(3-cyano-5-methyl-pyrazol-1-yl)-2-pyridyl]-6-[(6-methylpyridazin-3-yl)amino]benzimidazol-5-yl]oxypyrrolidine-1-carboxylic acid tert-butyl ester

Error! Objects cannot be created from editing field codes.

Prepared in analogy to example 104, step 2 using 3-[3-[5-acetyl-6-(3-cyano-5-methyl-pyrazol-1-yl)-2-pyridyl]-6-bromo-benzimidazol-5-yl]oxypyrrolidine-1-carboxylic acid tert-butyl ester (50 mg, 0.082 mmol, 1.0 equiv.) and (6-methylpyridazin-3-yl)amine (17.99 mg, 0.165 mmol, 2.0 equiv.) to afford the title compound (35.4 mg, 67.7% yield) as yellow solid. LC-MS: m/z=635.4 [M+H]⁺, ESI pos.

### Step 4: 3-[3-[6-(3-cyano-5-methyl-pyrazol-1-yl)-5-(1-hydroxyethyl)-2-pyridyl]-6-[(6-methylpyridazin-3-yl)amino]benzimidazol-5-yl]oxypyrrolidine-1-carboxylic acid tert-butyl ester

Error! Objects cannot be created from editing field codes.

Prepared in analogy to example 53, step 4 using 3-[3-[5-acetyl-6-(3-cyano-5-methyl-pyrazol-1-yl)-2-pyridyl]-6-[(6-methylpyridazin-3-yl)amino]benzimidazol-5-yl]oxypyrrolidine-1-carboxylic acid tert-butyl ester (35.4 mg, 0.056 mmol, 1 equiv.) in MeOH/THF (1:1) to afford the title compound (31.0 mg, 82.9% yield) as light yellow solid. LC-MS: 637.4 [M+H]⁺, ESI pos.

### Step 5: 1-[3-(-hydroxyethyl)-6-[5-[(6-methylpyridazin-3-yl)amino]-6-pyrrolidin-3-yloxy-benzimidazol-1-yl]-2-pyridyl]-5-methyl-pyrazole-3-carbonitrile

Error! Objects cannot be created from editing field codes.

To a stirred solution of 3-[3-[6-(3-cyano-5-methyl-pyrazol-1-yl)-5-(1-hydroxyethyl)-2-pyridyl]-6-[(6-methylpyridazin-3-yl)amino]benzimidazol-5-yl]oxypyrrolidine-1-carboxylic acid tert-butyl ester (31 mg, 0.049 mmol, 1 equiv.) at RT in DCM (0.231 mL) was added TFA (111 mg, 75 μL, 0.974 mmol, 20 equiv.). Stirring was continued at 23° C. for 2 hours. The reaction mixture was cooled to 0° C., 20 mL aq.

NaHCO₃ sol. (pH=8) was added carefully. The mixture was extracted with DCM, the organic layers washed with brine, dried over MgSO₄, and evaporated to dryness. The crude product was purified by falsh column chromatography (SiNH2,2-10% MeOH in DCM) to afford the title compound (17 0.0 mg, 60.5% yield) as light yellow solid. LC-MS: m/z=537.3 [M+H]⁺, ESI pos.

### Example 193

### 1-[3-(1-Hydroxyethyl)-6-[5-[[6-(2-methoxyethoxy)pyridazin-3-yl]amino]benzimidazol-1-yl]-2-pyridyl]-5-methyl-pyrazole-3-carbonitrile

Error! Objects cannot be created from editing field codes.

### Step 1: 3-chloro-6-(2-methoxyethoxy)pyridazine

Error! Objects cannot be created from editing field codes.

To a solution of 2-methoxyethanol (776.0 mg, 10.2 mmol, 0.990 equiv.) in THF (15 mL) was added NaH (492.95 mg, 12.32 mmol, 1.2 equiv.) in portions under nitrogen atmosphere at 0° C. The mixture was stirred at 0° C. for 30 min. Then 3,6-dichloropyridazine (1.53 g, 10.27 mmol, 1.0 equiv.) was added dropwise. The reaction mixture was stirred at 0° C. for another hour. The reaction mixture was quenched by addition of sat. aq. NH₄Cl at 0° C., and then diluted with EtOAc. The layers were separated and the aqueous layer washed further with EtOAc. The combined organic layers were filtered and concentrated under reduced pressure. The residue was purified by flash column chromatography (silica gel, 25% EtOAc in PE to yield 3-chloro-6-(2-methoxyethoxy)pyridazine (1.6 g, 8.48 mmol, 82.6% yield) as a colourless oil. ¹H NMR (400 MHz, CDCl₃) δ=7.44-7.33 (m, 1H), 7.12-6.99 (m, 1H), 4.73-4.57 (m, 2H), 3.87-3.74 (m, 2H), 3.45 (s, 3H).

### Step 2: tert-butyl N-[6-(2-methoxyethoxy)pyridazin-3-yl]carbamate

Error! Objects cannot be created from editing field codes.

To a brown turbid mixture of 3-chloro-6-(2-methoxyethoxy)pyridazine (1.0 g, 5.3 mmol, 1.0 equiv.), Pd₂(dba)₃ (485.51 mg, 0.530 mmol, 0.10 equiv.) in 1,4-dioxane (15 mL) were added tert-butyl carbamate (1.24 g, 10.6 mmol, 2.0 equiv.), Cs₂CO₃ (3.45 g, 10.6 mmol, 2.0 equiv.) and 9,9-dimethyl-4,5-bis(diphenylphosphino)xanthene (613.57 mg, 1.06 mmol, 0.20 equiv.). The reaction mixture was heated to 90° C. and stirred for 3 h under nitrogen atmosphere. The mixture was poured into water, and the aqueous phase was extracted with EtOAc. The combined organic layers were washed with, dried over Na₂SO₄ and concentrated under vacuum. The residue was purified by flash column chromatography (silica gel, 33% EtOAc in PE) to yield tert-butyl N-[6-(2-methoxyethoxy)pyridazin-3-yl]carbamate (800 mg, 2.97 mmol, 56.0% yield) as off-white solid. The reaction was successful. LC-MS: m/z=231.7 [M+H]⁺, ESI pos.

### Step 3: 6-(2-methoxyethoxy)pyridazin-3-amine; 2,2,2-trifluoroacetic acid

Error! Objects cannot be created from editing field codes.

A solution of tert-butyl N-[6-(2-methoxyethoxy)pyridazin-3-yl]carbamate (700.0 mg, 2.6 mmol, 1.0 equiv.) in trifluoroacetic acid (10.0 mL, 129.8 mmol, 50 equiv.) was stirred at 0° C. for 2 hours. The reaction mixture was concentrated under vacuum. The residue was triturated with PE/EtOAc (10:1) and filtered. The filter cake was dried to give 6-(2-methoxyethoxy)pyridazin-3-amine; 2,2,2-trifluoroacetic acid (650.9 mg, 2.3 mmol, 88.4% yield) as off-white solid. LC-MS: m/z=168.9 [M+H]⁺, ESI pos.

### Step 4: 1-[3-acetyl-6-[5-[[6-(2-methoxyethoxy)pyridazin-3-yl]amino]benzimidazol-1-yl]-2-pyridyl]-5-methyl-pyrazole-3-carbonitrile

Error! Objects cannot be created from editing field codes.

Prepared in analogy to example 104, step 2 using 1-[3-acetyl-6-(5-bromobenzimidazol-1-yl)-2-pyridyl]-5-methyl-pyrazole-3-carbonitrile (30 mg, 0.071 mmol, 1.0 equiv., prepared in example 65, step 1), [6-(2-methoxyethoxy)pyridazin-3-yl]amine; 2,2,2-trifluoroacetic acid (40.34 mg, 0.142 mmol, 2.0 equiv.) to afford the title compound (19.3 g, 50.5% yield) as yellow solid. LC-MS: m/z=510.3 [M+H]⁺, ESI pos.

### Step 5: 1-[3-(-hydroxyethyl)-6-[5-[[6-(2-methoxyethoxy)pyridazin-3-yl]amino]benzimidazol-1-yl]-2-pyridyl]-5-methyl-pyrazole-3-carbonitrile

Error! Objects cannot be created from editing field codes.

Prepared in analogy to example 53, step 4 using 1-[3-acetyl-6-[5-[[6-(2-methoxyethoxy)pyridazin-3-yl]amino]benzimidazol-1-yl]-2-pyridyl]-5-methyl-pyrazole-3-carbonitrile (19.3 mg, 0.036 mmol, 1.0 equiv.) in MeOH/THF (1:1) to afford the title compound (14.8 mg, 78.8% yield) as a white solid. LC-MS: m/z=512.3 [M+H]⁺, ESI pos.

### Example 194

### 1-[2-(1H-Benzotriazol-4-yl)-6-[5-[(6-methylpyridazin-3-yl)amino]benzimidazol-1-yl]-3-pyridyl]ethanol

Error! Objects cannot be created from editing field codes.

Prepared in analogy to example 163, step 3 using 1-[2-chloro-6-[5-[(6-methylpyridazin-3-yl)amino]benzimidazol-1-yl]-3-pyridyl]ethanol (30.0 mg, 0.080 mmol, 1.0 equiv., prepared in example 163, step 2) and 1H-benzotriazol-4-ylboronic acid (25.67 mg, 0.160 mmol, 2 equiv.) to yield 1-[2-(1H-benzotriazol-4-yl)-6-[5-[(6-methylpyridazin-3-yl)amino]benzimidazol-1-yl]-3-pyridyl]ethanol (1.3 mg, 3.2% yield) as a white solid. LC-MS: m/z=464.2 [M+H]⁺, ESI pos.

### Example 195

### 1-[6-[5-[[6-(2,2-Difluoroethoxy)pyridazin-3-yl]amino]benzimidazol-1-yl]-3-(1-hydroxyethyl)-2-pyridyl]-5-methyl-pyrazole-3-carbonitrile

Error! Objects cannot be created from editing field codes.

### Step 1: 3-chloro-6-(2,2-difluoroethoxy)pyridazine

Error! Objects cannot be created from editing field codes.

Prepared in analogy to example 193, step 1, using 2,2-difluoroethanol (600.0 mg, 7.31 mmol, 1.0 equiv.), sodium hydride (444.0 mg, 11.1 mmol, 1.5 equiv.), and 3,6-dichloropyridazine (1090.18 mg, 7.32 mmol, 1.0 equiv.) to afford 3-chloro-6-(2,2-difluoroethoxy)pyridazine (1000 mg, 5.14 mmol, 70.3% yield) as white solid. LC-MS: m/z=195.1[M+H]⁺, ESI pos.

### Step 2: tert-butyl N-[6-(2,2-difluoroethoxy)pyridazin-3-yl]carbamate

Error! Objects cannot be created from editing field codes.

Prepared in analogy to example 193, step 2, using 3-chloro-6-(2,2-difluoroethoxy)pyridazine (900.0 mg, 4.63 mmol, 1.0 equiv.) and tert-butyl carbamate (812.83 mg, 6.94 mmol, 1.5 equiv.) to afford tert-butyl N-[6-(2,2-difluoroethoxy)pyridazin-3-yl]carbamate (390 mg, 1.42 mmol, 30.6% yield) as white solid. LC-MS: m/z=220.1 [M-^{t}Bu+H]⁺, ESI pos.

### Step 3: 6-(2,2-difluoroethoxy)pyridazin-3-amine

Error! Objects cannot be created from editing field codes.

Prepared in analogy to example 193, step 3, using tert-butyl N-[6-(2,2-difluoroethoxy)pyridazin-3-yl]carbamate (370.0 mg, 1.34 mmol, 1.0 equiv.) to afford 6-(2,2-difluoroethoxy)pyridazin-3-amine (170.5 mg, 0.970 mmol, 72.42% yield) as white solid. LC-MS: m/z=176.1 [M+H]⁺, ESI pos.

### Step 4: 1-[3-acetyl-6-[5-[[6-(2,2-difluoroethoxy)pyridazin-3-yl]amino]benzimidazol-1-yl]-2-pyridyl]-5-methyl-pyrazole-3-carbonitrile

Error! Objects cannot be created from editing field codes.

Prepared in analogy to example 104, step 2 using 1-[3-acetyl-6-(5-bromobenzimidazol-1-yl)-2-pyridyl]-5-methyl-pyrazole-3-carbonitrile (30 mg, 0.071 mmol, 1.0 equiv., prepared in example 65, step 1) and [6-(2,2-difluoroethoxy)pyridazin-3-yl]amine (24.95 mg, 0.142 mmol, 2.0 equiv.) to afford the title compound (38.5 mg, 83.9% yield) as light yellow solid. LC-MS: m/z=516.3 [M+H]⁺, ESI pos.

### Step 5: 1-[6-[5-[[6-(2,2-difluoroethoxy)pyridazin-3-yl]amino]benzimidazol-1-yl]-3-(1-hydroxyethyl)-2-pyridyl]-5-methyl-pyrazole-3-carbonitrile

Error! Objects cannot be created from editing field codes.

Prepared in analogy to example 53, step 4, using 1-[3-acetyl-6-[5-[[6-(2,2-difluoroethoxy)pyridazin-3-yl]amino]benzimidazol-1-yl]-2-pyridyl]-5-methyl-pyrazole-3-carbonitrile (38.5 mg, 0.060 mmol, 1.0 equiv.) in MeOH/THF (1:1) to afford the title compound (22.1 mg, 67.9% yield) as light yellow solid. LC-MS: m/z=518.3 [M+H]⁺, ESI pos.

### Example 197

### 1-[6-[5-[[6-(Difluoromethoxy)pyridazin-3-yl]amino]benzimidazol-1-yl]-3-(1-hydroxyethyl)-2-pyridyl]-5-methyl-pyrazole-3-carbonitrile

Error! Objects cannot be created from editing field codes.

### Step 1: 6-(difluoromethoxy)pyridazin-3-amine

Error! Objects cannot be created from editing field codes.

To a solution of 6-aminopyridazin-3(2H)-one (1.24 g, 11.16 mmol, 1.0 equiv.) in CH₃CN (20 mL) was added 2,2-difluoro-2-(fluorosulfonyl)acetic acid (2.39 g, 13.39 mmol, 1.2 equiv.). The mixture was stirred at 20° C. for 48 h. The reaction solution was concentrated under reduced pressure and the crude was purified by preparative HPLC (Phenomenex luna C18 150 mm×25 mm×10 μm, gradient 1-15% CH₃CN in H₂O (with 0.225% formic acid) over 20 min, then 100% CH₃CN (2 min), flow rate 140 20 mL/min, 1 injection) to give the 6-(difluoromethoxy)pyridazin-3-amine (169.6 mg, 1.05 mmol, 9.2% yield) as brown gum. LC-MS: m/z=162.0 [M+H]⁺, ESI pos.

### Step 2: 1-[3-acetyl-6-[5-[[6-(difluoromethoxy)pyridazin-3-yl]amino]benzimidazol-1-yl]-2-pyridyl]-5-methyl-pyrazole-3-carbonitrile

Error! Objects cannot be created from editing field codes.

Prepared in analogy to example 104, step 2, using 1-[3-acetyl-6-(5-bromobenzimidazol-1-yl)-2-pyridyl]-5-methyl-pyrazole-3-carbonitrile (30 mg, 0.071 mmol, 1.0 equiv. prepared in example 65, step 1) and [6-(difluoromethoxy)pyridazin-3-yl]amine (22.95 mg, 0.142 mmol, 2.0 equiv.) to afford the title compound (24.1 mg, 64.1% yield) as yellow solid. LC-MS: m/z=502.3 [M+H]⁺, ESI pos.

### Step 3: 1-[6-[5-[[6-(difluoromethoxy)pyridazin-3-yl]amino]benzimidazol-1-yl]-3-(1-hydroxyethyl)-2-pyridyl]-5-methyl-pyrazole-3-carbonitrile

Error! Objects cannot be created from editing field codes.

Prepared in analogy to example 53, step 4 using 1-[3-acetyl-6-[5-[[6-(difluoromethoxy)pyridazin-3-yl]amino]benzimidazol-1-yl]-2-pyridyl]-5-methyl-pyrazole-3-carbonitrile (24.1 mg, 0.046 mmol, 1.0 equiv.) in MeOH/THF (1:1) to yield the title compound (19.3 mg, 79.8% yield) as off-white solid. LC-MS: m/z=504.3 [M+H]⁺, ESI pos.

### Example 198

### 1-[3-(2,2-Difluoro-1-hydroxy-ethyl)-6-[5-[(6-methylpyridazin-3-yl)amino]benzimidazol-1-yl]-2-pyridyl]-5-methyl-pyrazole-3-carbonitrile

Error! Objects cannot be created from editing field codes.

### Step 1: 1-[6-chloro-3-(4,4,4-trifluoro-3-oxo-butanoyl)-2-pyridyl]-5-methyl-pyrazole-3-carbonitrile

Error! Objects cannot be created from editing field codes.

To a solution of 1-(3-acetyl-6-chloro-2-pyridyl)-5-methyl-pyrazole-3-carbonitrile (3.53 g, 11.51 mmol, 1.0 equiv., prepared in example 64, step 1) in THF (35 mL) was added LiHMDS (23.0 mL, 23.02 mmol, 2.0 equiv.) dropwise at −70° C. The reaction mixture was stirred at −70° C. for 1 h. To the reaction mixture was added ethyl trifluoroacetate (1.64 g, 11.51 mmol, 1.0 equiv.) in THF (10 mL) dropwise at −70° C. The reaction mixture was stirred at RT for 2 hours. The reaction mixture was added into sat. aq.

NH₄Cl and extracted with EtOAc. The combined organic layers were concentrated under reduced pressure. The residue was purified by flash column chromatography (silica gel, 10-25% EtOAc in PE) to yield 1-[6-chloro-3-(4,4,4-trifluoro-3-oxo-butanoyl)-2-pyridyl]-5-methyl-pyrazole-3-carbonitrile (1.4 15 g, 3.92 mmol, 34.1% yield) as brown oil. LC-MS: m/z=357.1 [M+H]⁺, ESI pos.

### Step 2: 1-[6-chloro-3-(2,2-difluoroacetyl)-2-pyridyl]-5-methyl-pyrazole-3-carbonitrile

Error! Objects cannot be created from editing field codes.

A solution of 1-[6-chloro-3-(4,4,4-trifluoro-3-oxo-butanoyl)-2-pyridyl]-5-methyl-pyrazole-3-carbonitrile (3.2 g, 8.07 mmol, 1.0 equiv.) in CH₃CN (60 mL) was added 1-chloromethyl-4-fluoro-1,4-diazoniabicyclo[2.2.2]octane (7.15 g, 20.19 mmol, 2.5 equiv.) portionwise at 15° C. Then the mixture was stirred at 90° C. for 1 hour. The mixture was cooled to RT, and TEA (5.6 mL, 40.37 mmol, 5.0 equiv.) and water (0.29 mL, 16.15 mmol, 2.0 equiv.) were added at RT and stirred for 1.5 hours. The reaction mixture was diluted with water and extracted with EtOAc. The combined extracts were washed with brine, dried over Na₂SO₄ and concentrated to yield crude 1-[6-chloro-3-(2,2-difluoroacetyl)-2-pyridyl]-5-methyl-pyrazole-3-carbonitrile as brown oil which was used without further purification. LC-MS: m/z=296.9 [M+H]⁺, ESI pos.

### Step 3: 1-[6-chloro-3-(2,2-difluoro-1-hydroxy-ethyl)-2-pyridyl]-5-methyl-pyrazole-3-carbonitrile

Error! Objects cannot be created from editing field codes.

To a solution of 1-[6-chloro-3-(2,2-difluoroacetyl)-2-pyridyl]-5-methyl-pyrazole-3-carbonitrile (used as crude from the previous step) in THF (30 mL) was added NaBH₄ (918.21 mg, 24.27 mmol, 3.0 equiv.) at 0° C. in portions. After the addition was complete, the mixture was stirred at 0° C. for 1 h. The mixture was quenched with sat. aq. NH₄Cl and extracted with EtOAc. The combined organic layers were dried over Na₂SO₄ and concentrated. The crude product was purified by flash column chromatography (silica gel. 0-20% EtOAc in PE) to yield 1-[6-chloro-3-(2,2-difluoro-1-hydroxy-ethyl)-2-pyridyl]-5-methyl-pyrazole-3-carbonitrile (1.7 g, 5.69 mmol, 70.4% yield) as light yellow solid. LC-MS: m/z=299.0 [M+H]⁺, ESI pos.

### Step 4: 1-[3-(2,2-difluoro-1-hydroxy-ethyl)-6-[5-[(6-methylpyridazin-3-yl)amino]benzimidazol-1-yl]-2-pyridyl]-5-methyl-pyrazole-3-carbonitrile and 1-[3-(2,2-difluoro-1-hydroxy-ethyl)-6-[6-[(6-methylpyridazin-3-yl)amino]benzimidazol-1-yl]-2-pyridyl]-5-methyl-pyrazole-3-carbonitrile

Error! Objects cannot be created from editing field codes.

Prepared in analogy to example 64, step 2, using N-(6-methylpyridazin-3-yl)-1H-benzimidazol-5-amine (177.45 mg, 0.670 mmol, 1.0 equiv., prepared in example 64, intermediate 1) and 1-[6-chloro-3-(2,2-difluoro-1-hydroxy-ethyl)-2-pyridyl]-5-methyl-pyrazole-3-carbonitrile (200.0 mg, 0.670 mmol, 1.0 equiv.). The crude was purified by preparative HPLC (Phenomenex luna C18 (250×70 mm, 10 um), water (0.225% FA)-CH₃CN, 12-42% B, gradient time 20 min, 2 min 100% B hold time, flow rate 25 mL/min) followed by preparative NPLC (Waters Xbridge 150×25 mm×5 um), Hexane-EtOH (0.1% ammonium hydroxide), 10-50% B, gradient time 15 min, 5 min 100% B hold time, flow rate 100 mL/min, 1 injection) to separate the regioisomers. 1-[3-(2,2-difluoro-1-hydroxy-ethyl)-6-[5-[(6-methylpyridazin-3-yl)amino]benzimidazol-1-yl]-2-pyridyl]-5-methyl-pyrazole-3-carbonitrile (3.7 mg, 0.010 mmol, 1.13% yield) was obtained as white solid after further purification by preparative HPLC (Phenomenex luna C18 (250×70 mm, 10 um), water (10 mM NH₄HCO₃)—CH₃CN, 28-58% B, flow rate 25 mL/min, 1 injection). LC-MS: m/z=488.1 [M+H]⁺, ESI pos. ¹H NMR (400 MHz, DMSO-d₆) δ=9.25 (s, 1H), 9.05 (s, 1H), 8.50-8.42 (m, 2H), 8.32 (d, J=8.6 Hz, 1H), 8.06 (d, J=8.9 Hz, 1H), 7.55-7.46 (m, 1H), 7.33 (d, J=9.0 Hz, 1H), 7.15-7.03 (m, 2H), 6.84-6.58 (m, 1H), 6.32-5.95 (m, 1H), 4.86 (dt, J=3.4, 11.6 Hz, 1H), 2.48 (s, 3H), 2.36 (s, 3H).

### Example 199

### 1-[3-(2,2-Difluoro-1-hydroxy-ethyl)-6-[6-[(6-methylpyridazin-3-yl)amino]benzimidazol-1-yl]-2-pyridyl]-5-methyl-pyrazole-3-carbonitrile

Error! Objects cannot be created from editing field codes.

1-[3-(2,2-difluoro-1-hydroxy-ethyl)-6-[6-[(6-methylpyridazin-3-yl)amino]benzimidazol-1-yl]-2-pyridyl]-5-methyl-pyrazole-3-carbonitrile (6.1 mg, 0.010 mmol, 1.8% yield) was obtained as yellow solid after separation of the regioisomers by preparative NPLC (Waters Xbridge 150×25 mm×5 um), Hexane-EtOH (0.1% ammonium hydroxide), 10-50% B, gradient time 15 min, 5 min 100% B hold time, flow rate 100 mL/min, 1 injection) in example 198, step 4. LC-MS: m/z=488.3 [M+H]⁺, ESI pos. ¹H NMR (400 MHz, CD₃OD) δ=8.83 (br s, 2H), 8.61 (d, J=8.5 Hz, 1H), 8.20 (d, J=8.5 Hz, 1H), 7.70 (d, J=8.5 Hz, 1H), 7.45 (dd, J=1.7, 8.6 Hz, 1H), 7.40-7.31 (m, 1H), 7.13 (br d, J=9.1 Hz, 1H), 6.82 (s, 1H), 6.16-5.80 (m, 1H), 5.08-5.00 (m, 1H), 2.56 (s, 3H), 2.41 (s, 3H).

### Example 201

### 1-[6-[5-[(1,1-Dioxo-1,2-thiazolidin-2-yl)methyl]benzimidazol-1-yl]-3-(1-hydroxyethyl)-2-pyridyl]-5-methyl-pyrazole-3-carbonitrile

Error! Objects cannot be created from editing field codes.

### Step 1: 2-[[5-[(1,1-dioxo-1,2-thiazolidin-2-yl)methyl]benzimidazol-1-yl]methoxy]ethyl-trimethyl-silane

Error! Objects cannot be created from editing field codes.

To a solution of 2-[[5-(chloromethyl)benzimidazol-1-yl]methoxy]ethyl-trimethyl-silane (500.0 mg, 1.68 mmol, 1.0 equiv., prepared in example 212, step 3) in DMSO (10 mL) was added 1,3-propanesultam (245.0 mg, 2.02 mmol, 1.2 equiv.) and K₂CO₃ (698.29 mg, 5.05 mmol, 3 equiv.). The reaction mixture was stirred at 30° C. for 2 h. The reaction mixture was diluted with water and was extracted with EtOAc. The organic layer was washed with brine and concentrated under reduced pressure to yield 2-[[5-[(1,1-dioxo-1,2-thiazolidin-2-yl)methyl]benzimidazol-1-yl]methoxy]ethyl-trimethyl-silane (600 mg, 1.57 mmol, 93.4% yield) as yellow oil. LC-MS: m/z=382.2 [M+H]⁺, ESI pos.

### Step 2: 2-(1H-benzimidazol-5-ylmethyl)-1,2-thiazolidine 1,1-dioxide

Error! Objects cannot be created from editing field codes.

A solution of 2-[[5-[(1,1-dioxo-1,2-thiazolidin-2-yl)methyl]benzimidazol-1-yl]methoxy]ethyl-trimethyl-silane (500.0 mg, 1.31 mmol, 1.0 equiv.) in TFA (5.0 mL) was stirred at 20° C. for 2 h. The reaction mixture was concentrated under reduced pressure. The residue was purified by preparative TLC (10% MeOH in DCM) to yield 2-(1H-benzimidazol-5-ylmethyl)-1,2-thiazolidine 1,1-dioxide (250 mg, 0.990 mmol, 75.9% yield) as colorless oil. LC-MS: m/z=252.0 [M+H]⁺, ESI pos.

### Step 3: 1-[3-acetyl-6-[6-[(1,1-dioxo-1,2-thiazolidin-2-yl)methyl]benzimidazol-1-yl]-2-pyridyl]-5-methyl-pyrazole-3-carbonitrile and 1-[3-acetyl-6-[5-[(1,1-dioxo-1,2-thiazolidin-2-yl)methyl]benzimidazol-1-yl]-2-pyridyl]-5-methyl-pyrazole-3-carbonitrile

Error! Objects cannot be created from editing field codes.

Prepared in analogy to example 64, step 2, using 1-(3-acetyl-6-chloro-2-pyridyl)-5-methyl-pyrazole-3-carbonitrile (248.96 mg, 0.960 mmol, 1.0 equiv., prepared in example 64, step 1) and 2-(1H-benzimidazol-5-ylmethyl)-1,2-thiazolidine 1,1-dioxide (240.0 mg, 0.960 mmol, 1.0 equiv.) to give 1-[3-acetyl-6-[5-[(1,1-dioxo-1,2-thiazolidin-2-yl)methyl]benzimidazol-1-yl]-2-pyridyl]-5-methyl-pyrazole-3-carbonitrile (150 mg, 0.320 mmol, 33.0% yield) as yellow oil and 1-[3-acetyl-6-[6-[(1,1-dioxo-1,2-thiazolidin-2-yl)methyl]benzimidazol-1-yl]-2-pyridyl]-5-methyl-pyrazole-3-carbonitrile (150 mg, 0.320 mmol, 33.0% yield) as yellow oil after separation by preparative TLC (10% MeOH in DCM). Regioisomer 1: LC-MS: m/z=476.1 [M+H]⁺, ESI pos. ¹H NMR (400 MHz, DMSO-d₆) δ=9.15 (s, 1H), 8.57 (br d, J=8.7 Hz, 1H), 8.36-8.22 (m, 2H), 7.79 (br d, J=7.3 Hz, 1H), 7.37 (br d, J=7.9 Hz, 1H), 7.15 (s, 1H), 4.23 (s, 2H), 3.26-3.20 (m, 2H), 3.09 (br t, J=6.2 Hz, 2H), 2.57 (s, 3H), 2.26-2.12 (m, 5H). Regioisomer 2: LC-MS: m/z=476.1 [M+H]⁺, ESI pos. ¹H NMR (400 MHz, DMSO-d₆) δ=9.15 (s, 1H), 8.56 (br d, J=8.2 Hz, 1H), 8.31 (br d, J=8.3 Hz, 1H), 8.20 (br d, J=8.4 Hz, 1H), 7.78 (s, 1H), 7.40 (br d, J=8.7 Hz, 1H), 7.13 (s, 1H), 4.23 (s, 2H), 3.26 (br t, J=7.5 Hz, 2H), 3.11 (br t, J=6.3 Hz, 2H), 2.53 (s, 3H), 2.21 (s, 5H).

### Step 4: 1-[6-[5-[(1,1-dioxo-1,2-thiazolidin-2-yl)methyl]benzimidazol-1-yl]-3-(1-hydroxyethyl)-2-pyridyl]-5-methyl-pyrazole-3-carbonitrile

Error! Objects cannot be created from editing field codes.

Prepared in analogy to example 53, step 4, using 1-[3-acetyl-6-[5-[(1,1-dioxo-1,2-thiazolidin-2-yl)methyl]benzimidazol-1-yl]-2-pyridyl]-5-methyl-pyrazole-3-carbonitrile (150.0 mg, 0.320 mmol, 1.0 equiv.) in MeOH/THF (1:1) to afford 1-[6-[5-[(1,1-dioxo-1,2-thiazolidin-2-yl)methyl]benzimidazol-1-yl]-3-(1-hydroxyethyl)-2-pyridyl]-5-methyl-pyrazole-3-carbonitrile (41.3 mg, 0.090 mmol, 26.8% yield) as white solid. LC-MS: m/z=478.2 [M+H]⁺, ESI pos. ¹H NMR (400 MHz, DMSO-d₆) δ=9.07 (s, 1H), 8.48 (d, J=8.4 Hz, 1H), 8.29 (d, J=8.4 Hz, 1H), 8.11 (d, J=8.4 Hz, 1H), 7.77 (d, J=0.7 Hz, 1H), 7.37 (dd, J=1.4, 8.5 Hz, 1H), 7.11 (d, J=0.7 Hz, 1H), 5.56 (d, J=4.4 Hz, 1H), 4.63-4.54 (m, 1H), 4.23 (s, 2H), 3.30-3.24 (m, 2H), 3.10 (t, J=6.7 Hz, 2H), 2.35 (s, 3H), 2.26-2.17 (m, 2H), 1.27 (d, J=6.4 Hz, 3H).

### Example 202

### 1-[6-[6-[(1,1-Dioxo-1,2-thiazolidin-2-yl)methyl]benzimidazol-1-yl]-3-(1-hydroxyethyl)-2-pyridyl]-5-methyl-pyrazole-3-carbonitrile

Error! Objects cannot be created from editing field codes.

Prepared in analogy to example 53, step 4, using 1-[3-acetyl-6-[6-[(1,1-dioxo-1,2-thiazolidin-2-yl)methyl]benzimidazol-1-yl]-2-pyridyl]-5-methyl-pyrazole-3-carbonitrile (150.0 mg, 0.320 mmol, 1.0 equiv., obtained in example 201, step 3) MeOH/THF (1:1) to afford 1-[6-[6-[(1,1-dioxo-1,2-thiazolidin-2-yl)methyl]benzimidazol-1-yl]-3-(1-hydroxyethyl)-2-pyridyl]-5-methyl-pyrazole-3-carbonitrile (43.2 mg, 0.090 mmol, 28.7% yield) as white solid. LC-MS: m/z=478.2 [M+H]⁺, ESI pos. ¹H NMR (400 MHz, DMSO-d₆) δ=9.05 (s, 1H), 8.48 (d, J=8.6 Hz, 1H), 8.27 (d, J=8.4 Hz, 1H), 8.14 (s, 1H), 7.76 (d, J=8.3 Hz, 1H), 7.32 (dd, J=1.4, 8.3 Hz, 1H), 7.10 (d, J=0.6 Hz, 1H), 5.55 (d, J=4.4 Hz, 1H), 4.64-4.55 (m, 1H), 4.20 (s, 2H), 3.25-3.18 (m, 2H), 3.06 (t, J=6.7 Hz, 2H), 2.37 (s, 3H), 2.22-2.09 (m, 2H), 1.27 (d, J=6.5 Hz, 3H).

### Example 203

### 3-[3-(1-Hydroxyethyl)-6-[5-[(6-methylpyridazin-3-yl)amino]benzimidazol-1-yl]-2-pyridyl]-2-methoxy-phenol; formic acid

Error! Objects cannot be created from editing field codes.

### Step 1: 1-[2-(3-hydroxy-2-methoxy-phenyl)-6-[5-[(6-methylpyridazin-3-yl)amino]benzimidazol-1-yl]-3-pyridyl]ethanone

Error! Objects cannot be created from editing field codes.

Prepared in analogy to example 143, step 2 using 1-[2-chloro-6-[5-[(6-methylpyridazin-3-yl)amino]benzimidazol-1-yl]-3-pyridyl]ethanone (80.0 mg, 0.210 mmol, 1.0 equiv., prepared in example 76, step 2), and 2-methoxy-3-(4,4,5,5-tetramethyl-1,3,2-dioxaborolan-2-yl)phenol (316.9 mg, 1.27 mmol, 6 equiv.), to afford 1-[2-(3-hydroxy-2-methoxy-phenyl)-6-[5-[(6-methylpyridazin-3-yl)amino]benzimidazol-1-yl]-3-pyridyl]ethanone (20 mg, 0.040 mmol, 20.3% yield) as a yellow solid. LC-MS: m/z=467.3, [M+H]⁺, ESI pos.

### Step 2:3-[3-(1-hydroxyethyl)-6-[5-[(6-methylpyridazin-3-yl)amino]benzimidazol-1-yl]-2-pyridyl]-2-methoxy-phenol; formic acid

Error! Objects cannot be created from editing field codes.

Prepared in analogy to example 53, step 4 using 1-[2-(3-hydroxy-2-methoxy-phenyl)-6-[5-[(6-methylpyridazin-3-yl)amino]benzimidazol-1-yl]-3-pyridyl]ethanone (20.0 mg, 0.040 mmol, 1.0 equiv) to afford 3-[3-(1-hydroxyethyl)-6-[5-[(6-methylpyridazin-3-yl)amino]benzimidazol-1-yl]-2-pyridyl]-2-methoxy-phenol; formic acid (3 mg, 0.010 mmol, 12.2% yield) as a yellow solid. LC-MS: m/z=469.2, [M+H]⁺, ESI pos.

### Example 204

### 3-[3-(1-Hydroxyethyl)-6-[5-[(6-methylpyridazin-3-yl)amino]benzimidazol-1-yl]-2-pyridyl]furan-2-carbonitrile

Error! Objects cannot be created from editing field codes.

### Step 1: 3-[3-acetyl-6-[5-[(6-methylpyridazin-3-yl)amino]benzimidazol-1-yl]-2-pyridyl]furan-2-carbonitrile

Error! Objects cannot be created from editing field codes.

Prepared in analogy to example 143, step 2 using 1-[2-chloro-6-[5-[(6-methylpyridazin-3-yl)amino]benzimidazol-1-yl]-3-pyridyl]ethanone (50.0 mg, 0.130 mmol, 1.0 equiv., prepared in example 65, step 1) and (2-cyano-3-furyl)boronic acid (35.6 mg, 0.260 mmol, 2.0 equiv.) to afford 3-[3-acetyl-6-[5-[(6-methylpyridazin-3-yl)amino]benzimidazol-1-yl]-2-pyridyl]furan-2-carbonitrile (12 mg, 0.030 mmol, 20.9% yield) as yellow solid. LC-MS: m/z=436.1 [M+H]⁺, ESI pos.

### Step 2: 3-[3-(1-hydroxyethyl)-6-[5-[(6-methylpyridazin-3-yl)amino]benzimidazol-1-yl]-2-pyridyl]furan-2-carbonitrile

Error! Objects cannot be created from editing field codes.

Prepared in analogy to example 53, step 4 using 3-[3-acetyl-6-[5-[(6-methylpyridazin-3-yl)amino]benzimidazol-1-yl]-2-pyridyl]furan-2-carbonitrile (10.0 mg, 0.020 mmol, 1.0 equiv.) and NaBH₄ (4.37 mg, 0.110 mmol, 5. Equiv.) to afford 3-[3-(1-hydroxyethyl)-6-[5-[(6-methylpyridazin-3-yl)amino]benzimidazol-1-yl]-2-pyridyl]furan-2-carbonitrile (3.3 mg, 0.010 mmol, 32.3% yield) as white solid. LC-MS: m/z=438.1 [M+H]⁺, ESI pos. ¹H NMR (400 MHz, CD₃OD) δ=8.95 (s, 1H), 8.38 (d, J=8.8 Hz, 1H), 8.27-8.17 (m, 2H), 8.06-7.95 (m, 2H), 7.68-7.58 (m, 1H), 7.35 (d, J=9.2 Hz, 1H), 7.18-7.09 (m, 2H), 5.24-5.11 (m, 1H), 2.53 (s, 3H), 1.53 (d, J=6.4 Hz, 3H).

### Example 205

### 1-[3-(1-Hydroxyethyl)-6-[5-[(6-methylpyridazin-3-yl)amino]benzimidazol-1-yl]-2-pyridyl]-4,5,6,7-tetrahydropyrazolo[4,3-c]pyridine-3-carbonitrile

Error! Objects cannot be created from editing field codes.

### Step 1: 5-tert-butyl 3-ethyl 6,7-dihydro-1H-pyrazolo[4,3-c]pyridine-3,5(4H)-dicarboxylate

Error! Objects cannot be created from editing field codes.

Di-tert.-butyl-dicarbonate (1.04 g, 4.76 mmol, 1.0 equiv.) was added to an ice-cold suspension of ethyl 4,5,6,7-tetrahydro-1H-pyrazolo[4,3-c]pyridine-3-carboxylate (930 mg, 4.76 mmol, 1.0 equiv.) in Et₂O (45 ml). After the addition the reaction mixture was stirred at RT overnight. Water was added and the mixture was extracted with EtOAc. The organic layers were washed with water and brine, dried over Na₂SO₄ and evaporated. The crude was purified by flash column chromatography (silica gel, 30-55% EtOAc in heptane) to yield the title compound (1.08 g, 76.7% yield) as white solid. LC-MS: m/z=396.0 [M+H]⁺, 240.2 [M-Boc+H]⁺.

### Step 2: 5-tert-butoxycarbonyl-1,4,6,7-tetrahydropyrazolo[4,3-c]pyridine-3-carboxylic acid

Error! Objects cannot be created from editing field codes.

To a stirred, almost clear solution 5-tert-butyl 3-ethyl 6,7-dihydro-1H-pyrazolo[4,3-c]pyridine-3,5(4H)-dicarboxylate (1.25 g, 4.23 mmol, 1.0 equiv.) at RT in a mixture of methanol (6.5 mL) and tetrahydrofuran (6.5 mL) under an argon atmosphere was added 1 M NaOH (12.7 mL, 12.7 mmol, 3.0 equiv.). Stirring was continued overnight. The mixture was treated with 1N HCl (12.2 ml) and diluted with H₂O (25 ml), and the reaction mixture was stirred at RT for another 1 h 30, yielding a suspension. The product was collected by filtration, washed thoroughly with H₂O and dried under reduced pressure to yield 5-tert-butoxycarbonyl-1,4,6,7-tetrahydropyrazolo[4,3-c]pyridine-3-carboxylic acid (997 mg, 86.4% yield) as off-white solid. LC-MS: m/z=266.2 [M−H]−, ESI neg.

### Step 3: 3-carbamoyl-1,4,6,7-tetrahydropyrazolo[4,3-c]pyridine-5-carboxylic acid tert-butyl ester

Error! Objects cannot be created from editing field codes.

To a stirred solution of 5-tert-butoxycarbonyl-1,4,6,7-tetrahydropyrazolo[4,3-c]pyridine-3-carboxylic acid (955 mg, 3.22 mmol, 1.0 equiv.) at RT in DMF (15 mL) under an argon atmosphere was added CDI (677.86 mg, 4.18 mmol, 1.3 equiv.). After stirring for 3 hours at RT, NH₄OH (2.25 g, 2.5 mL, 64.31 mmol, 20 equiv.) was added and stirring was continued for 6 hours. H₂O was added yielding an off-white slurry which was was stirred at RT overnight. The solid was collected by filtration, washed thoroughly with H₂O and dried to yield 3-carbamoyl-1,4,6,7-tetrahydropyrazolo[4,3-c]pyridine-5-carboxylic acid tert-butyl ester (775 mg, 88.7% yield) as a white solid. LC-MS: m/z=265.2 [M−H]⁻, ESI neg.

### Step 4: 3-cyano-1,4,6,7-tetrahydropyrazolo[4,3-c]pyridine-5-carboxylic acid tert-butyl ester

Error! Objects cannot be created from editing field codes.

A suspension of 3-carbamoyl-1,4,6,7-tetrahydropyrazolo[4,3-c]pyridine-5-carboxylic acid tert-butyl ester (770 mg, 2.75 mmol, 1.0 equiv.) and Et₃N (833.9 mg, 1.15 mL, 8.24 mmol, 3.0 equiv.) in DCM (20 mL) under an argon atmosphere was cooled to 0° C. and TFAA (1.73 g, 1.16 mL, 8.24 mmol, 3 equiv.) was slowly. The reaction mixture was allowed to stir at 0° C. for 1 h. The reaction mixture was adsorbed onto silica gel and purified by flash column chromatography (silica gel, 0-15% MeOH in DCM). The resulting light brown paste was suspended in Et₂O and stirred at RT overnight. The solid was collected by filtration, washed with Et₂O and dried to yield the title compound (490 mg, 68.3% yield) as a white solid. LC-MS: m/z=247.2 [M−H]⁻, ESI neg.

### Step 5: 1-(3-acetyl-6-chloro-2-pyridyl)-3-cyano-6,7-dihydro-4H-pyrazolo[4,3-c]pyridine-5-carboxylic acid tert-butyl ester

Error! Objects cannot be created from editing field codes.

Prepared in analogy to example 62, step 1 using 3-cyano-1,4,6,7-tetrahydropyrazolo[4,3-c]pyridine-5-carboxylic acid tert-butyl ester (480 mg, 1.84 mmol, 1.0 equiv.) and 1-(6-chloro-2-fluoro-3-pyridyl)ethanone (318.78 mg, 1.84 mmol, 1.0 equiv.) to afford the title compound (550 mg, 70.8% yield) as white foam. LC-MS: m/z=402.1 [M+H]⁺, ESI pos.

### Step 6: 1-[3-acetyl-6-[5-[(6-methylpyridazin-3-yl)amino]benzimidazol-1-yl]-2-pyridyl]-3-cyano-6,7-dihydro-4H-pyrazolo[4,3-c]pyridine-5-carboxylic acid tert-butyl ester and 1-[3-acetyl-6-[6-[(6-methylpyridazin-3-yl)amino]benzimidazol-1-yl]-2-pyridyl]-3-cyano-6,7-dihydro-4H-pyrazolo[4,3-c]pyridine-5-carboxylic acid tert-butyl ester

Error! Objects cannot be created from editing field codes.

To a stirred solution of 1-(3-acetyl-6-chloro-2-pyridyl)-3-cyano-6,7-dihydro-4H-pyrazolo[4,3-c]pyridine-5-carboxylic acid tert-butyl ester (700 mg, 1.74 mmol, 1.0 eqiuv.) at RT in DMSO (7 mL) under an argon atmosphere were added 1H-benzimidazol-5-yl-(6-methylpyridazin-3-yl)amine (435.97 mg, 1.74 mmol, 1 equiv., prepared in example 64, intermediate 1) and DABCO (397.79 mg, 393.85 μL, 2.61 mmol, 1.5 equiv.). The mixture was heated to 85° C. and stirring at that temperature was continued for 5 h. The mixture was allowed to cool to RT, diluted with EtOAc and washed with H₂O. The aqueous layer was back-extracted with EtOAc. The combined organic layers were washed with H₂O and brine, dried over MgSO4 and the volatiles removed. The residue was purified by flash column chromatography (silica gel, 0-15% MeOH) twice to separate both regioisomers to yield 1-[3-acetyl-6-[5-[(6-methylpyridazin-3-yl)amino]benzimidazol-1-yl]-2-pyridyl]-3-cyano-6,7-dihydro-4H-pyrazolo[4,3-c]pyridine-5-carboxylic acid tert-butyl ester (213 mg, 20.3% yield) as light brown solid and 1-[3-acetyl-6-[6-[(6-methylpyridazin-3-yl)amino]benzimidazol-1-yl]-2-pyridyl]-3-cyano-6,7-dihydro-4H-pyrazolo[4,3-c]pyridine-5-carboxylic acid tert-butyl ester (250 mg, 23.8% yield) as a light brown solid. Regioisomer 1: LC-MS: m/z=591.3 [M+H]⁺, ESI pos. ¹H NMR (600 MHz, DMSO-d₆) δ ppm 9.26 (s, 1H), 9.05 (s, 1H), 8.46 (s, 1H), 8.45 (s, 1H), 8.19-8.23 (m, 1H), 8.10 (d, J=8.7 Hz, 1H), 7.51 (br s, 1H), 7.34 (d, J=9.1 Hz, 1H), 7.09 (d, J=9.1 Hz, 1H), 4.59 (s, 2H), 3.68 (t, J=5.7 Hz, 2H), 3.10 (br t, J=5.5 Hz, 2H), 2.48 (s, 3H), 2.27 (s, 3H), 1.37-1.55 (m, 9H). Regioisomer 2: LC-MS: m/z=591.3 [M+H]⁺, ESI pos. ¹H NMR (600 MHz, DMSO-d₆) δ ppm 9.32 (s, 1H), 9.25 (d, J=1.7 Hz, 1H), 8.93 (s, 1H), 8.47 (d, J=8.4 Hz, 1H), 8.15 (d, J=8.4 Hz, 1H), 7.70 (d, J=8.7 Hz, 1H), 7.39 (dd, J=8.8, 2.1 Hz, 1H), 7.30 (br d, J=8.8 Hz, 1H), 7.05 (d, J=9.0 Hz, 1H), 4.52 (s, 2H), 3.46 (t, J=5.7 Hz, 2H), 3.02-3.20 (m, 2H), 2.46 (s, 3H), 2.28 (s, 3H), 1.20-1.42 (m, 9H)

### Step 7: 3-cyano-1-[3-(1-hydroxyethyl)-6-[5-[(6-methylpyridazin-3-yl)amino]benzimidazol-1-yl]-2-pyridyl]-6,7-dihydro-4H-pyrazolo[4,3-c]pyridine-5-carboxylic acid tert-butyl ester

Error! Objects cannot be created from editing field codes.

Prepared in analogy to example 53, step 4 using 1-[3-acetyl-6-[5-[(6-methylpyridazin-3-yl)amino]benzimidazol-1-yl]-2-pyridyl]-3-cyano-6,7-dihydro-4H-pyrazolo[4,3-c]pyridine-5-carboxylic acid tert-butyl ester (53 mg, 0.088 mmol, 1.0 equiv.) in MeOH/THF (1:1) to afford the title compound (44 mg, 80.2% yield) as light brown solid. LC-MS: m/z=593.3 [M+H]⁺, ESI pos.

### Step 8: 1-[3-(1-hydroxyethyl)-6-[5-[(6-methylpyridazin-3-yl)amino]benzimidazol-1-yl]-2-pyridyl]-4,5,6,7-tetrahydropyrazolo[4,3-c]pyridine-3-carbonitrile

Error! Objects cannot be created from editing field codes.

To a stirred light brown solution of 3-cyano-1-[3-(1-hydroxyethyl)-6-[5-[(6-methylpyridazin-3-yl)amino]benzimidazol-1-yl]-2-pyridyl]-6,7-dihydro-4H-pyrazolo[4,3-c]pyridine-5-carboxylic acid tert-butyl ester (41 mg, 0.066 mmol, 1.0 equiv.) at RT in 1,4-dioxane (1.7 mL) under an argon atmosphere was added 4 M HCl in dioxane (394.3 mg, 329 μL, 1.31 mmol, 20 equiv.), and stirring at r.t. was continued for 1 h45. The mixture was concentrated to dryness and purified by (Phenomenex Gemini NX C18 (100 mm×30 mm×5 μm). Flow rate: 30 mL/min. Gradient: 15% to 45% CH₃CN in (0.1% TEA in H2O) (8 min) then 100% CH₃CN (2 min). to yield the title compound (22 mg, 66.6% yield) as off-white solid. LC-MS: m/z=493.3 [M+H]⁺, ESI pos.

### Example 206

### 1-[6-[5-[[6-[2-(1,1-Diketo-1,4-thiazinan-4-yl)ethoxy]pyridazin-3-yl]amino]benzimidazol-1-yl]-3-(1-hydroxyethyl)-2-pyridyl]-5-methyl-pyrazole-3-carbonitrile

Error! Objects cannot be created from editing field codes.

### Step 1: 4-[2-(6-chloropyridazin-3-yl)oxyethyl]-1,4-thiazinane 1,1-dioxide

Error! Objects cannot be created from editing field codes.

Prepared in analogy to example 193, step 1 using 4-(2-hydroxyethyl)thiomorpholine 1,1-dioxide (1.2 g, 6.7 mmol, 1.0 equiv.) and 3,6-dichloropyridazine (1.0 g, 6.7 mmol, 1.0 equiv.) to yield 4-[2-(6-chloropyridazin-3-yl)oxyethyl]-1,4-thiazinane 1,1-dioxide (400 mg, 1.37 mmol, 20.4% yield) as off-white solid. LC-MS: m/z=292.0 [M+H]⁺, ESI pos.

### Step 2: tert-butyl N-[6-[2-(1,1-dioxo-1,4-thiazinan-4-yl)ethoxy]pyridazin-3-yl]carbamate

Error! Objects cannot be created from editing field codes.

Prepared in analogy to example 193, step 2 using 4-[2-(6-chloropyridazin-3-yl)oxyethyl]-1,4-thiazinane 1,1-dioxide (330.0 mg, 1.13 mmol, 1.0 equiv.) and tert-butyl carbamate (265.02 mg, 2.26 mmol, 2 equiv.), Cs₂CO₃ (737.07 mg, 2.26 mmol, 2 equiv.) to provide tert-butyl N-[6-[2-(1,1-dioxo-1,4-thiazinan-4-yl)ethoxy]pyridazin-3-yl]carbamate (300 mg, 0.810 mmol, 71.2% yield) as off-white solid. LC-MS: m/z=373.1 [M+H]⁺, ESI pos.

### Step 3: 6-[2-(1,1-dioxo-1,4-thiazinan-4-yl)ethoxy]pyridazin-3-amine

Error! Objects cannot be created from editing field codes.

Prepared in analogy to example 193, step 3 using tert-butyl N-[6-[2-(1,1-dioxo-1,4-thiazinan-4-yl)ethoxy]pyridazin-3-yl]carbamate (300.0 mg, 0.810 mmol, 1.0 equiv.) in DCM (6 mL) were added trifluoroacetic acid (3.0 mL, 38.94 mmol, 48.34 equiv.) to afford 6-[2-(1,1-dioxo-1,4-thiazinan-4-yl)ethoxy]pyridazin-3-amine (161 mg, 0.590 mmol, 73.4% yield) as white solid. LC-MS: m/z=273.0 [M+H]⁺, ESI pos.

### Step 4: 1-[3-acetyl-6-[5-[[6-[2-(1,1-diketo-1,4-thiazinan-4-yl)ethoxy]pyridazin-3-yl]amino]benzimidazol-1-yl]-2-pyridyl]-5-methyl-pyrazole-3-carbonitrile

Error! Objects cannot be created from editing field codes.

Prepared in analogy to example 104, step 2 using 1-[3-acetyl-6-(5-bromobenzimidazol-1-yl)-2-pyridyl]-5-methyl-pyrazole-3-carbonitrile (30 mg, 0.071 mmol, 1.0 equiv., prepared in example 65, step 1) and[6-[2-(1,1-diketo-1,4-thiazinan-4-yl)ethoxy]pyridazin-3-yl]amine (38.79 mg, 0.142 mmol, 2.0 equiv.)to afford the title compound (21.2 mg, 46.2% yield) as yellow solid. LC-MS: m/z=613.3 [M+H]⁺, ESI pos.

### Step 5: 1-[6-[5-[[6-[2-(1,1-diketo-1,4-thiazinan-4-yl)ethoxy]pyridazin-3-yl]amino]benzimidazol-1-yl]-3-(1-hydroxyethyl)-2-pyridyl]-5-methyl-pyrazole-3-carbonitrile

Error! Objects cannot be created from editing field codes.

Prepared in analogy to example 53, step 4 using 1-[3-acetyl-6-[5-[[6-[2-(1,1-diketo-1,4-thiazinan-4-yl)ethoxy]pyridazin-3-yl]amino]benzimidazol-1-yl]-2-pyridyl]-5-methyl-pyrazole-3-carbonitrile (21.2 mg, 0.033 mmol, 1.0 equiv.) on MeOH/THF (1:1) to afford the title compound (11.1 mg, 53.3% yield). LC-MS: m/z=615.3 [M+H]⁺, ESI pos.

### Example 207

### 1-[3-(1-Hydroxyethyl)-6-[5-[[6-(oxetan-3-yloxy)pyridazin-3-yl]amino]benzimidazol-1-yl]-2-pyridyl]-5-methyl-pyrazole-3-carbonitrile

Error! Objects cannot be created from editing field codes.

### Step 1: 3-chloro-6-(oxetan-3-yloxy)pyridazine

Error! Objects cannot be created from editing field codes.

Prepared in analogy to example 93, step 1 using oxetan-3-ol (493.8 mg, 6.7 mmol, 1.0 equiv.) and 3,6-dichloropyridazine (1.0 g, 6.7 mmol, 1.0 equiv.) to afford 3-chloro-6-(oxetan-3-yloxy)pyridazine (1.0 g, 5.36 mmol, 79.8% yield) as off-white solid. LC-MS: m/z=187.0 [M+H]⁺, ESI pos.

### Step 2: tert-butyl N-[6-(oxetan-3-yloxy)pyridazin-3-yl]carbamate

Error! Objects cannot be created from editing field codes.

Prepared in analogy to example 193, step 2 using 3-chloro-6-(oxetan-3-yloxy)pyridazine (1.0 g, 5.36 mmol, 1.0 equiv.) and tert-butyl carbamate (1.26 g, 10.72 mmol, 2.0 equiv.). LC-MS: m/z=268.0 [M+H]⁺, ESI pos.

### Step 3: 6-(oxetan-3-yloxy)pyridazin-3-amine

Error! Objects cannot be created from editing field codes.

Prepared in analogy to example 193, step 3 using tert-butyl N-[6-(oxetan-3-yloxy)pyridazin-3-yl]carbamate (400.0 mg, 1.5 mmol, 1.0 equiv.) to provide 6-(oxetan-3-yloxy)pyridazin-3-amine (102 mg, 0.610 mmol, 36.7% yield) as white solid. ). LC-MS: m/z=168.0 [M+H]⁺, ESI pos.

### Step 4: 1-[3-acetyl-6-[5-[[6-(oxetan-3-yloxy)pyridazin-3-yl]amino]benzimidazol-1-yl]-2-pyridyl]-5-methyl-pyrazole-3-carbonitrile

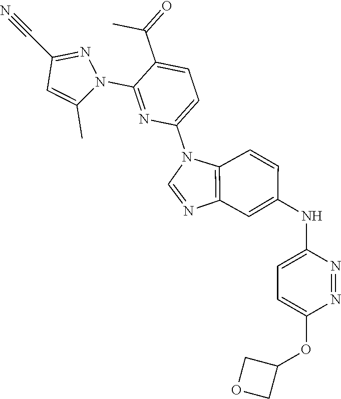 [SMILES: CC(=O)c1ccc(-n2cnc3cc(Nc4ccc(OC5COC5)nn4)ccc32)nc1-n1nc(C#N)cc1C]

Prepared in analogy to example 104, step 2 using 1-[3-acetyl-6-(5-bromobenzimidazol-1-yl)-2-pyridyl]-5-methyl-pyrazole-3-carbonitrile (30 mg, 0.071 mmol, 1.0 equiv., prepared in example 65, step 1) and [6-(oxetan-3-yloxy)pyridazin-3-yl]amine (23.81 mg, 0.142 mmol, 2 equiv.) to afford the title compound (23.7 mg, 62.3% yield) as a yellow solid. LC-MS: m/z=508.3 [M+H]⁺, ESI pos.

### Step 5: 1-[3-(1-hydroxyethyl)-6-[5-[[6-(oxetan-3-yloxy)pyridazin-3-yl]amino]benzimidazol-1-yl]-2-pyridyl]-5-methyl-pyrazole-3-carbonitrile

Error! Objects cannot be created from editing field codes.

Prepared in analogy to example 53, step 4 using 1-[3-acetyl-6-[5-[[6-(oxetan-3-yloxy)pyridazin-3-yl]amino]benzimidazol-1-yl]-2-pyridyl]-5-methyl-pyrazole-3-carbonitrile (23.7 mg, 0.047 mmol, 1.0 equiv.) in MeOH/THF (1:1) to afford the title compound (14.3 mg, 57.1% yield) as light yellow solid. LC-MS: m/z=510.3 [M+H]⁺, ESI pos.

### Example 208

### 1-[6-[5-[(6-Methylpyridazin-3-yl)amino]benzimidazol-1-yl]-2-[(3S)-pyrrolidin-3-yl]oxy-3-pyridyl]ethanol

Error! Objects cannot be created from editing field codes.

### Step 1: 6-chloro-2-hydroxy-nicotinic acid methyl ester

Error! Objects cannot be created from editing field codes.

Under an argon atmosphere was dissolved 6-chloro-2-hydroxy-nicotinic acid (1.0 g, 5.76 mmol, 1.0 equiv.) in DCM (15 mL) and cooled to 0° C. before SOCl₂ (2.06 g, 1.25 mL, 17.29 mmol, 3.0 equiv.) was added. After 5 min at 0° C., the ice bath was removed and the reaction was stirred for 1.5 h at RT. MeOH (184.6 mg, 233 μL, 5.76 mmol, 1.0 equiv.) was added and the reaction was stirred for 30 min. The reaction mixture was concentrated in vacuo and purified by flash column chromatography (silica gel, 0-100% EtOAc in heptane) to obtain 6-chloro-2-hydroxy-nicotinic acid methyl ester (710 mg, 65%) as off-white solid. LC-MS: m/z=188.02 [M+H]⁺, ESI pos.

### Step 2: 2-[(3 S)-1-tert-butoxycarbonylpyrrolidin-3-yl]oxy-6-chloro-nicotinic acid methyl ester

Error! Objects cannot be created from editing field codes.

Under an argon atmosphere were added 6-chloro-2-hydroxy-nicotinic acid methyl ester (150 mg, 0.800 mmol, 1 equiv.), (3R)-3-hydroxypyrrolidine-1-carboxylic acid tert-butyl ester (179.7 mg, 0.960 mmol, 1.2 equiv.) and PPh₃ (251.7 mg, 0.960 mmol, 1.2 equiv.). The compounds were dissolved in THF (3 mL). DIAD (198 mg, 190 μL, 0.960 mmol, 1.2 equiv.) was added. After 1 h stirring at RT, another portion of (3R)-3-hydroxypyrrolidine-1-carboxylic acid tert-butyl ester (179.7 mg, 0.960 mmol, 1.2 eq uiv.), PPh₃ (251.7 mg, 0.960 mmol, 1.2 equiv.) and DIAD (198 mg, 190 μL, 0.960 mmol, 1.2 equiv.) were added, and the mixture stirred for another h. The mixture was diluted with water and extracted with EtOAc. The organic layers were combined, dried over MgSO₄ and concentrated under reduced pressure. The crude was purified by flash column chromatography (silica gel, 0-100% EtOAc in heptane) to obtain the title compound 2-[(3S)-1-tert-butoxycarbonylpyrrolidin-3-yl]oxy-6-chloro-nicotinic acid methyl ester (247.6 mg, 86.3%) as light brown solid. LC-MS: m/z=301.04 [M-^{t}Bu+H]⁺, ESI pos.

### Step 3: 2-[(3S)-1-tert-butoxycarbonylpyrrolidin-3-yl]oxy-6-chloro-nicotinic acid

Error! Objects cannot be created from editing field codes.

To a stirred suspension of 6-chloro-2-[(3S)-1-tert-butoxycarbonylpyrrolidin-3-yl]oxy-nicotinic acid methyl ester (240 mg, 0.673 mmol, 1.0 equiv.) in a mixture of MeOH (3.2 mL), THF (3.2 mL) and water (5.2 mL) was added under an argon atmosphere and at RT 1 M NaOH (1.35 mL, 1.35 mmol, 2 eq). Stirring at RT was continued overnight. The mixture was heated to 65° C. and stirring was continued for 6 hours. The mixture was cooled to 0° C. before 1N HCl (1.35 ml) was added. The mixture was diluted with water and extracted with EtOAc. The combined organic layers were dried over MgSO₄ and the volatiles evaporated to yield 2-[(3S)-1-tert-butoxycarbonylpyrrolidin-3-yl]oxy-6-chloro-nicotinic acid as off-white solid which was used in the next step without further purification. LC-MS: m/z=341.21 [M−H]⁻, ESI neg.

### Step 4: (3S)-3-[[6-chloro-3-[methoxy(methyl)carbamoyl]-2-pyridyl]oxy]pyrrolidine-1-carboxylic acid tert-butyl ester

Error! Objects cannot be created from editing field codes.

To a stirred solution of 6-chloro-2-[(3S)-1-tert-butoxycarbonylpyrrolidin-3-yl]oxy-nicotinic acid (used as crude from the previous step) at RT in DMF (2 mL) under an argon atmosphere were added HATU (221.0 mg, 0.581 mmol, 1.2 equiv.) and DIPEA (187.8 mg, 248.7 μL, 1.45 mmol, 3.0 equiv.). After stirring for 5 minutes, N,O-dimethylhydroxylamine hydrochloride (56.7 mg, 0.581 mmol, 1.2 equiv.) was added and the mixture was stirred at RT for further 90 minutes. The mixture was diluted with H₂O upon which a precipitate formed and the yellow suspension was stirred for 1 hour. The solid was then collected by filtration, washed well with H₂O and dried under reduced pressure to yield the title compound (153 mg, 81.9% yield). LC-MS: m/z=286.12 [M-Boc+H]⁺, ESI pos.

### Step 5: (3S)-3-[(3-acetyl-6-chloro-2-pyridyl)oxy]pyrrolidine-1-carboxylic acid tert-butyl ester

Error! Objects cannot be created from editing field codes.

To a stirred suspension of (3S)-3-[[6-chloro-3-[methoxy(methyl)carbamoyl]-2-pyridyl]oxy]pyrrolidine-1-carboxylic acid tert-butyl ester (130 mg, 0.337 mmol, 1.0 equiv.) at 0° C. in THF (3.25 mL) under an argon atmosphere was added MeMgBr (3.2 M in 2-MeTHF, 126 μL, 0.40 mmol, 1.2 equiv.). The cooling bath was removed and stirring at RT was continued for 15 minutes. Another portion of MeMgBr (3.2 M in 2-MeTHF, 126 μL, 0.40 mmol, 1.2 equiv.) was added and stirring at RT was continued overnight. The mixture was treated carefully with H₂O extracted with EtOAc. The combined organic layers were washed with water and brine, dried over MgSO₄ and concentrated to obtain crude (3S)-3-[(3-acetyl-6-chloro-2-pyridyl)oxy]pyrrolidine-1-carboxylic acid tert-butyl ester as brown solid which was used in the next step without further purification. LC-MS: m/z=285.10 [M-tBu+H]⁺, ESI pos.

### Step 6: (3S)-3-[[3-acetyl-6-[5-[(6-methylpyridazin-3-yl)amino]benzimidazol-1-yl]-2-pyridyl]oxy]pyrrolidine-1-carboxylic acid tert-butyl ester and (3S)-3-[[3-acetyl-6-[6-[(6-methylpyridazin-3-yl)amino]benzimidazol-1-yl]-2-pyridyl]oxy]pyrrolidine-1-carboxylic acid tert-butyl ester

Error! Objects cannot be created from editing field codes.

Prepared in analogy to Example 64, step 2 using (3S)-3-[(3-acetyl-6-chloro-2-pyridyl)oxy]pyrrolidine-1-carboxylic acid tert-butyl ester (used as crude from the previous step), 1H-benzimidazol-5-yl-(6-methylpyridazin-3-yl)amine (87.24 mg, 0.387 mmol, 1.0 equiv., example 64, intermediate 1) and K₂CO₃ (160.6 mg, 1.16 mmol, 3.0 equiv.) to yield (3S)-3-[[3-acetyl-6-[5-[(6-methylpyridazin-3-yl)amino]benzimidazol-1-yl]-2-pyridyl]oxy]pyrrolidine-1-carboxylic acid tert-butyl ester as an off-white solid (70 mg, 34.1% yield) and (3S)-3-[[3-acetyl-6-[6-[(6-methylpyridazin-3-yl)amino]benzimidazol-1-yl]-2-pyridyl]oxy]pyrrolidine-1-carboxylic acid tert-butyl ester as an off-white solid (42 mg, 20.3% yield). LC-MS: m/z=423.2 [M+H]⁺, ESI pos. Regioisomer 1: 1H NMR (600 MHz, DMSO-d₆) δ ppm 9.27 (s, 1H), 9.05 (s, 1H), 8.35 (br d, J=3.9 Hz, 1H), 8.30 (br d, J=8.2 Hz, 1H), 8.23 (br dd, J=8.6, 5.7 Hz, 1H), 7.63-7.69 (m, 2H), 7.36 (d, J=9.1 Hz, 1H), 7.11 (d, J=9.1 Hz, 1H), 5.79-5.94 (m, 1H), 3.62-3.78 (m, 2H), 3.53 (br s, 2H), 2.56 (s, 3H), 2.31 (br d, J=1.1 Hz, 2H), 1.40 (br d, J=18.4 Hz, 9H). Regioisomer 2: 1H NMR (600 MHz, DMSO-d₆) δ ppm 9.54 (br s, 1H), 9.35 (s, 1H), 8.96 (s, 1H), 8.23-8.45 (m, 1H), 7.62-7.79 (m, 2H), 7.37 (d, J=9.1 Hz, 1H), 7.20 (br d, J=8.6 Hz, 1H), 7.12 (s, 1H), 6.03-6.35 (m, 1H), 3.65-3.97 (m, 1H), 3.51-3.67 (m, 1H), 2.58 (s, 3H), 2.15-2.38 (m, 2H), 1.36 (br d, J=17.3 Hz, 9H).

### Step 7: (3S)-3-[[3-(1-hydroxyethyl)-6-[5-[(6-methylpyridazin-3-yl)amino]benzimidazol-1-yl]-2-pyridyl]oxy]pyrrolidine-1-carboxylic acid tert-butyl ester

Error! Objects cannot be created from editing field codes.

Prepared in analogy to example 53, step 4, using (3S)-3-[[3-acetyl-6-[5-[(6-methylpyridazin-3-yl)amino]benzimidazol-1-yl]-2-pyridyl]oxy]pyrrolidine-1-carboxylic acid tert-butyl ester (42.7 mg, 0.081 mmol, 1.0 equiv.) in THF/iPrOH (1:1) to yield (3S)-3-[[3-(1-hydroxyethyl)-6-[5-[(6-methylpyridazin-3-yl)amino]benzimidazol-1-yl]-2-pyridyl]oxy]pyrrolidine-1-carboxylic acid tert-butyl ester (29.9 mg, 69.8%) as white solid. LC-MS: m/z=532.22 [M+H]⁺, ESI pos.

### Step 8: 1-[6-[5-[(6-methylpyridazin-3-yl)amino]benzimidazol-1-yl]-2-[(3S)-pyrrolidin-3-yl]oxy-3-pyridyl]ethanol

Error! Objects cannot be created from editing field codes.

(3S)-3-[[3-(1-hydroxyethyl)-6-[5-[(6-methylpyridazin-3-yl)amino]benzimidazol-1-yl]-2-pyridyl]oxy]pyrrolidine-1-carboxylic acid tert-butyl ester (29.9 mg, 0.056 mmol, 1.0 equiv.) was dissolved in DCM (0.6 mL). At 0° C. TFA (128.3 mg, 86.7 μL, 1.12 mmol, 20.0 equiv.) was added. The ice bath was removed and the reaction was stirred at RT for 2 hours. The mixture was concentrated in vacuo and purified by reversed phase column chromatography (silica gel C18, 10-100% CH₃CN in H₂O) to obtain the title compound 1-[6-[5-[(6-methylpyridazin-3-yl)amino]benzimidazol-1-yl]-2-[(3S)-pyrrolidin-3-yl]oxy-3-pyridyl]ethanol (9.7 mg, 40.0%) as off-white solid. LC-MS: m/z=432.27 [M+H]⁺, ESI pos.

### Example 212

### 1-[3-(1-Hydroxyethyl)-6-[6-[(2-oxopyrrolidin-1-yl)methyl]benzimidazol-1-yl]-2-pyridyl]-5-methyl-pyrazole-3-carbonitrile

Error! Objects cannot be created from editing field codes.

### Step 1: methyl 1-(2-trimethylsilylethoxymethyl)benzimidazole-5-carboxylate

Error! Objects cannot be created from editing field codes.

To a solution of methyl 1H-benzimidazole-5-carboxylate (3.0 g, 17.03 mmol, 1.0 equiv.) in THF (50 mL) was added sodium hydride (1.02 g, 25.54 mmol, 1.5 equiv.) slowly at 0° C., and the reaction mixture was stirred at 0° C. for 1 h. 2-(trimethylsilyl)ethoxymethyl chloride (3.92 mL, 22.14 mmol, 1.3 equiv.) was added slowly at 0° C., the cooling bath was removed and the reaction mixture was stirred at RT for 2 hours. The reaction was poured into water EtOAc. The organic phase was washed with brine, dried with anhydrous Na₂SO₄, filtered and concentrated to dryness in vacuo. The residue was purified by flash column chromatography (silica gel, 10-50% EtOAc in PE) to afford methyl 1-(2-trimethylsilylethoxymethyl)benzimidazole-5-carboxylate (3.8 g, 12.4 mmol, 72.8% yield) as light brown oil. LC-MS: m/z=307.2 [M+H]⁺, ESI pos.

### Step 2: [1-(2-trimethylsilylethoxymethyl)benzimidazol-5-yl]methanol

Error! Objects cannot be created from editing field codes.

To a solution of methyl 1-(2-trimethylsilylethoxymethyl)benzimidazole-5-carboxylate (6.5 g, 21.2 mmol, 1.0 equiv.) in THF (65 mL) was added LiAlH₄ (1.28 g, 32.1 mmol, 1.5 equiv.) in portions at −10° C. The reaction mixture was stirred at −10° C. for 1.5 h. After the completion of the reaction, it was added dropwise water (0.53 mL) at −15° C. under stirring, followed by NaOH (15%, 0.53 mL). The mixture was filtered, the mother liquor was concentrated under reduced pressure. The residue was purified by flash column chromatography (silica gel, 70% EtOAc in PE) to give [1-(2-trimethylsilylethoxymethyl)benzimidazol-5-yl]methanol (5.0 g, 18.0 mmol, 84.7% yield) as yellow oil. LC-MS: m/z=279.2 [M+H]⁺, ESI pos.

### Step 3: 2-[[5-(chloromethyl)benzimidazol-1-yl]methoxy]ethyl-trimethyl-silane

Error! Objects cannot be created from editing field codes.

To the solution of [1-(2-trimethylsilylethoxymethyl)benzimidazol-5-yl]methanol (2.5 g, 9.0 mmol, 1.0 equiv.) and DIPEA (4.68 mL, 27.0 mmol, 3.0 equiv.) in DCM (40 mL) was added MsCl (1.39 mL, 18.0 mmol, 2.0 equiv.) dropwise at 0° C. The reaction mixture was stirred at RT for 12 hours. The reaction mixture was poured into aqueous NaHCO₃ under stirring and was extracted with DCM. The combined extracts were concentrated under vacuum and purified by flash column chromatography (silica gel, 40% EtOAc in PE) to yield 2-[[5-(chloromethyl)benzimidazol-1-yl]methoxy]ethyl-trimethyl-silane (1.7 g, 5.73 mmol, 63.7% yield) as yellow oil. LC-MS: m/z=297.1 [M+H]⁺, ESI pos.

### Step 4: 1-[[1-(2-trimethylsilylethoxymethyl)benzimidazol-5-yl]methyl]pyrrolidin-2-one

Error! Objects cannot be created from editing field codes.

To a solution of 2-pyrrolidone (0.23 mL, 3.03 mmol, 1.5 equiv.) in DMF (20 mL) was added sodium hydride (162.0 mg, 4.05 mmol, 2.0 equiv.) in portions. The mixture was stirred at 0° C. for 30 min. Then 2-[[5-(chloromethyl)benzimidazol-1-yl]methoxy]ethyl-trimethyl-silane (600.0 mg, 2.02 mmol, 1.0 equiv.) was added dropwise as a solution in DMF (2 mL). The reaction mixture was stirred at 20° C. for 1 h. The reaction mixture was poured into aqueous NH₄Cl solution under stirring and was extracted with EtOAc. The organic phase was washed with brine and the volatiles evaporated. The residue was purified by flash column chromatography (silica gel, 10% MeOH in EtOAc) and concentrated under reduced pressure to give 1-[[1-(2-trimethylsilylethoxymethyl)benzimidazol-5-yl]methyl]pyrrolidin-2-one (450 mg, 1.3 mmol, 64.44% yield) as yellow oil. LC-MS: m/z=346.1 [M+H]⁺, ESI pos.

### Step 5: 1-(1H-benzimidazol-5-ylmethyl)pyrrolidin-2-one

Error! Objects cannot be created from editing field codes.

TFA (3 mL) was added to 1-[[1-(2-trimethylsilylethoxymethyl)benzimidazol-5-yl]methyl]pyrrolidin-2-one (430.0 mg, 1.24 mmol, 1.0 equiv.) at 0° C., the cooling bath removed and the reaction was further stirred at RT for 2 hours. The mixture was concentrated to yield crude 1-(1H-benzimidazol-5-ylmethyl)pyrrolidin-2-one as yellow oil which was used in the next step without further purification. LC-MS: m/z=216.1 [M+H]⁺, ESI pos.

### Step 6: 1-[3-acetyl-6-[6-[(2-oxopyrrolidin-1-yl)methyl]benzimidazol-1-yl]-2-pyridyl]-5-methyl-pyrazole-3-carbonitrile and 1-[3-acetyl-6-[6-[(2-oxopyrrolidin-1-yl)methyl]benzimidazol-1-yl]-2-pyridyl]-5-methyl-pyrazole-3-carbonitrile

Error! Objects cannot be created from editing field codes.

Prepared in analogy to example 64, step 2 using 1-(3-acetyl-6-chloro-2-pyridyl)-5-methyl-pyrazole-3-carbonitrile (270.0 mg, 1.04 mmol, 1.0 equiv., prepared in example 64, step 1), 1-(1H-benzimidazol-5-ylmethyl)pyrrolidin-2-one (used as crude from the previous step) and K₂CO₃ (429.42 mg, 3.11 mmol, 3 equiv.) to yield 1-[3-acetyl-6-[5-[(2-oxopyrrolidin-1-yl)methyl]benzimidazol-1-yl]-2-pyridyl]-5-methyl-pyrazole-3-carbonitrile (45 mg, 0.100 mmol, 9.9% yield) as yellow oil and 1-[3-acetyl-6-[6-[(2-oxopyrrolidin-1-yl)methyl]benzimidazol-1-yl]-2-pyridyl]-5-methyl-pyrazole-3-carbonitrile (30 mg, 0.070 mmol, 6.59% yield) as yellow solid after purification and separation of both regioisomers by preparative TLC (10% MeOH in DCM). Regioisomer 1: LC-MS: m/z=440.2 [M+H]⁺, ESI pos. ¹H NMR (400 MHz, CDCl₃) δ=2.23 (s, 3H) 2.42-2.52 (m, 3H) 2.63 (s, 3H) 3.33 (t, J=7.03 Hz, 2H) 4.13 (q, J=7.21 Hz, 2H) 4.63 (s, 2H) 6.73 (s, 1H) 7.34-7.40 (m, 1H) 7.74-7.81 (m, 2H) 7.98-8.03 (m, 1H) 8.31 (d, J=8.31 Hz, 1H) 8.65 (s, 1H). Regioisomer 2: LC-MS: m/z=440.2 [M+H]⁺, ESI pos ¹H NMR (400 MHz, CDCl₃) δ=1.94-1.99 (m, 2H) 2.23 (s, 3H) 2.38-2.43 (m, 2H) 2.63 (s, 3H) 3.28-3.33 (m, 3H) 4.59-4.63 (m, 2H) 6.74 (s, 1H) 7.31 (d, J=8.19 Hz, 1H) 7.77-7.82 (m, 1H) 7.85 (d, J=8.31 Hz, 1H) 7.94-7.98 (m, 1H) 8.30-8.36 (m, 1H) 8.64 (s, 1H).

### Step 7: 1-[3-(-hydroxyethyl)-6-[6-[(2-oxopyrrolidin-1-yl)methyl]benzimidazol-1-yl]-2-pyridyl]-5-methyl-pyrazole-3-carbonitrile

Error! Objects cannot be created from editing field codes.

Prepared in analogy to example 53, step 4 using 1-[3-acetyl-6-[6-[(2-oxopyrrolidin-1-yl)methyl]benzimidazol-1-yl]-2-pyridyl]-5-methyl-pyrazole-3-carbonitrile (25.0 mg, 0.060 mmol, 1.0 equiv.) and NaBH₄ (6.46 mg, 0.170 mmol, 3.0 equiv.) to provide 1-[3-(1-hydroxyethyl)-6-[6-[(2-oxopyrrolidin-1-yl)methyl]benzimidazol-1-yl]-2-pyridyl]-5-methyl-pyrazole-3-carbonitrile (4.1 mg, 0.010 mmol, 16.3% yield) as colorless oil after preparative HPLC (Phenomenex Synergi C18 150×25 mm×10 μm, gradient 29-45% CH₃CN in H₂O (with formic acid) over 8 min, then 100% CH₃CN (2 min), flow rate 25 mL/min, 1 injection). LC-MS: m/z=442.1, [M+H]⁺, ESI pos. ¹H NMR (400 MHz, CD₃OD) δ=8.96 (s, 1H), 8.53 (d, J=8.5 Hz, 1H), 8.17 (d, J=8.5 Hz, 1H), 8.10 (d, J=0.9 Hz, 1H), 7.77 (d, J=8.4 Hz, 1H), 7.34 (dd, J=1.6, 8.3 Hz, 1H), 6.91 (d, J=0.8 Hz, 1H), 4.78 (d, J=6.5 Hz, 1H), 4.62 (d, J=3.4 Hz, 2H), 3.38-3.34 (m, 2H), 2.41 (d, J=0.6 Hz, 3H), 2.38 (d, J=8.5 Hz, 2H), 2.00-1.92 (m, 2H), 1.43 (d, J=6.5 Hz, 3H).

### Example 213

### 1-[3-(1-Hydroxyethyl)-6-[5-[(2-oxopyrrolidin-1-yl)methyl]benzimidazol-1-yl]-2-pyridyl]-5-methyl-pyrazole-3-carbonitrile

Error! Objects cannot be created from editing field codes.

Prepared in analogy to example 53, step 4 using 1-[3-acetyl-6-[5-[(2-oxopyrrolidin-1-yl)methyl]benzimidazol-1-yl]-2-pyridyl]-5-methyl-pyrazole-3-carbonitrile (45.0 mg, 0.100 mmol, 1.0 equiv., prepared in example 212, step 6) and NaBH₄ (11.62 mg, 0.310 mmol, 3.0 equiv.) to provide 1-[3-(1-hydroxyethyl)-6-[5-[(2-oxopyrrolidin-1-yl)methyl]benzimidazol-1-yl]-2-pyridyl]-5-methyl-pyrazole-3-carbonitrile (6 mg, 0.010 mmol, 13.27% yield) as white solid after preparative HPLC (Phenomenex Synergi C18 150×25 mm×10 μm, gradient 27-45% CH₃CN in H₂O (with formic acid) over 9 min, then 100% CH₃CN (2 min), flow rate 25 mL/min, 1 injection). LC-MS: m/z=442.1, [M+H]⁺, ESI pos. ¹H NMR (400 MHz, CD₃OD) δ=8.97 (s, 1H), 8.54 (d, J=8.3 Hz, 1H), 8.18 (dd, J=2.4, 8.4 Hz, 2H), 7.70 (s, 1H), 7.39-7.30 (m, 1H), 6.88 (s, 1H), 4.79 (d, J=6.2 Hz, 1H), 4.63 (s, 2H), 3.40 (t, J=7.1 Hz, 2H), 2.48 (t, J=8.0 Hz, 2H), 2.42 (s, 3H), 2.09-2.00 (m, 2H), 1.43 (d, J=6.5 Hz, 3H).

### Example 214

### 1-[2-(3-Methoxy-5-methyl-pyrazol-1-yl)-6-[5-[(6-methylpyridazin-3-yl)amino]benzimidazol-1-yl]-3-pyridyl]ethanol; formic acid

Error! Objects cannot be created from editing field codes.

### Step 1: 2-(3-methoxy-5-methyl-pyrazol-1-yl)-6-[5-[(6-methylpyridazin-3-yl)amino]benzimidazol-]-yl]pyridine-3-carboxylic acid

Error! Objects cannot be created from editing field codes.

To a solution of methyl 2-(3-methoxy-5-methyl-pyrazol-1-yl)-6-[5-[(6-methylpyridazin-3-yl)amino]benzimidazol-1-yl]pyridine-3-carboxylate (200.0 mg, 0.430 mmol, 1.0 equiv., prepared in Example 153, step 2) in THF (10 mL), MeOH (10 mL) and water (10 mL) was added LiOH H₂O monohydrate (20.0 mg, 0.480 mmol, 1.12 equiv.). The mixture was stirred at 25° C. for 4 h. The solution was acidified to pH 7 with aqueous HCl (1N) and concentrated in vacuo to give 2-(3-methoxy-5-methyl-pyrazol-1-yl)-6-[5-[(6-methylpyridazin-3-yl)amino]benzimidazol-1-yl]pyridine-3-carboxylic acid as light yellow solid. The crude product was used in the next step without further purification. LC-MS: m/z=457.3 [M+H]⁺, ESI pos.

### Step 2: N-methoxy-2-(3-methoxy-5-methyl-pyrazol-1-yl)-N-methyl-6-[5-[(6-methylpyridazin-3-yl)amino]benzimidazol-1-yl]pyridine-3-carboxamide

Error! Objects cannot be created from editing field codes.

To a solution of 2-(3-methoxy-5-methyl-pyrazol-1-yl)-6-[5-[(6-methylpyridazin-3-yl)amino]benzimidazol-1-yl]pyridine-3-carboxylic acid (used as crude from the previous step) in DMF (4 mL) were added O,N-dimethylhydroxylamine HCl (42.0 mg, 0.430 mmol, 1.2 equiv.) and DIPEA (0.12 mL, 0.710 mmol, 2.1 equiv.) in this order at 0° C. Finally HATU (158.0 mg, 0.420 mmol, 1.2 equiv.) was added. The pH was confirmed to be at 5-6. The reaction mixture was stirred at 25° C. for 16 h. Then, the reaction mixture was quenched with aqueous NH₄Cl, and extracted with EtOAc. The combined organic layers were dried over Na₂SO₄, filtered and concentrated under reduced pressure. The residue was purified by preparative TLC (silica gel, 10% MeOH in DCM) to give N-methoxy-2-(3-methoxy-5-methyl-pyrazol-1-yl)-N-methyl-6-[5-[(6-methylpyridazin-3-yl)amino]benzimidazol-1-yl]pyridine-3-carboxamide (120 mg, 0.240 mmol, 69.4% yield) as a light yellow solid. LC-MS: m/z=500.3 [M+H]⁺, ESI pos.

### Step 3: 1-[2-(3-methoxy-5-methyl-pyrazol-1-yl)-6-[5-[(6-methylpyridazin-3-yl)amino]benzimidazol-]-yl]-3-pyridyl]ethanone

Error! Objects cannot be created from editing field codes.

N-methoxy-2-(3-methoxy-5-methyl-pyrazol-1-yl)-N-methyl-6-[5-[(6-methylpyridazin-3-yl)amino]benzimidazol-1-yl]pyridine-3-carboxamide (120.0 mg, 0.240 mmol, 1.0 equiv.) was dissolved in THF (15 mL). Methyl magnesium bromide (20.0 mL, 60 mmol, 250 equiv.) was added dropwise the mixture at 0° C. After addition, the mixture was stirred at 0° C. for 1 h. As a yellow solid precipitated out after 1 h, the mixture was further diluted with (15 mL) and stirred at 25° C. for 3 h. The resulting yellow suspension was quenched with aqueous NH₄Cl and extracted with EtOAc. The combined organic layers were dried over Na₂SO₄, filtered and concentrated under reduced pressure. The residue was purified by preparative TLC (silica gel, 10% MeOH in DCM) to give 1-[2-(3-methoxy-5-methyl-pyrazol-1-yl)-6-[5-[(6-methylpyridazin-3-yl)amino]benzimidazol-1-yl]-3-pyridyl]ethanone (30 mg, 0.070 mmol, 19.2% yield) as a light yellow solid. LC-MS: m/z=455.2 [M+H]⁺, ESI pos.

### Step 4: 1-[2-(3-methoxy-5-methyl-pyrazol-1-yl)-6-[5-[(6-methylpyridazin-3-yl)amino]benzimidazol-1-yl]-3-pyridyl]ethanol; formic acid

Error! Objects cannot be created from editing field codes.

Prepared in analogy to Example 53, step 4 using 1-[2-(3-methoxy-5-methyl-pyrazol-1-yl)-6-[5-[(6-methylpyridazin-3-yl)amino]benzimidazol-1-yl]-3-pyridyl]ethanone (20.0 mg, 0.03 mmol, 1.0 equiv.) and NaBH₄ (6.0 mg, 0.160 mmol, 5.2 equiv.) to yield 1-[2-(3-methoxy-5-methyl-pyrazol-1-yl)-6-[5-[(6-methylpyridazin-3-yl)amino]benzimidazol-1-yl]-3-pyridyl]ethanol; formic acid (4.2 mg, 0.010 mmol, 24.4% yield) as light yellow solid. LC-MS: m/z=457.2 [M+H]⁺, ESI pos. ¹H NMR (400 MHz, CD₃OD) δ=8.86 (s, 1H), 8.41 (d, J=8.4 Hz, 1H), 8.34 (br s, 1H), 8.21 (d, J=2.0 Hz, 1H), 8.15 (d, J=9.0 Hz, 1H), 7.97 (d, J=8.4 Hz, 1H), 7.60 (dd, J=1.7, 8.9 Hz, 1H), 7.36 (d, J=9.1 Hz, 1H), 7.14 (d, J=9.1 Hz, 1H), 5.83 (s, 1H), 5.07 (q, J=6.5 Hz, 1H), 3.90 (s, 3H), 2.53 (s, 3H), 2.35 (s, 3H), 1.43 (d, J=6.5 Hz, 3H).

### Example 215

### 1-[3-(1-Hydroxyethyl)-6-[6-[(2-oxo-1-piperidyl)methyl]benzimidazol-1-yl]-2-pyridyl]-5-methyl-pyrazole-3-carbonitrile

Error! Objects cannot be created from editing field codes.

### Step 1: 1-[[1-(2-trimethylsilylethoxymethyl)benzimidazol-5-yl]methyl]piperidin-2-one

Error! Objects cannot be created from editing field codes.

Prepared in analogy to example 212, step 3 using 2-piperidone (400.0 mg, 4.0 mmol, 1.2 equiv.) and 2-[[5-(chloromethyl)benzimidazol-1-yl]methoxy]ethyl-trimethyl-silane (1.0 g, 3.37 mmol, 1.0 equiv., prepared in example 212, step 2) to give 1-[[1-(2-trimethylsilylethoxymethyl)benzimidazol-5-yl]methyl]piperidin-2-one (200 mg, 0.560 mmol, 16.5% yield) as a colorless oil. LC-MS: m/z=360.2 [M+H]⁺, ESI pos.

### Step 2: 1-(1H-benzimidazol-5-ylmethyl)piperidin-2-one

Error! Objects cannot be created from editing field codes.

Prepared in analogy to example 212, step 4 using 1-[[1-(2-trimethylsilylethoxymethyl)benzimidazol-5-yl]methyl]piperidin-2-one (180.0 mg, 0.500 mmol, 1.0 equiv.) to give 1-(1H-benzimidazol-5-ylmethyl)piperidin-2-one as a colorless oil which was used directly in the next step without further purification. LC-MS: m/z=230.0 [M+H]⁺, ESI pos.

### Step 3: 1-[3-acetyl-6-[5-[(2-oxo-1-piperidyl)methyl]benzimidazol-1-yl]-2-pyridyl]-5-methyl-pyrazole-3-carbonitrile and 1-[3-acetyl-6-[5-[(2-oxo-1-piperidyl)methyl]benzimidazol-1-yl]-2-pyridyl]-5-methyl-pyrazole-3-carbonitrile

Error! Objects cannot be created from editing field codes.

Prepared in analogy to example Example 64, step 2 using 1-(3-acetyl-6-chloro-2-pyridyl)-5-methyl-pyrazole-3-carbonitrile (120.0 mg, 0.460 mmol, 1.0 equiv., prepared in Example 64, step 1) and 1-(1H-benzimidazol-5-ylmethyl)piperidin-2-one (used as crude from the previous step) to yield 1-[3-acetyl-6-[6-[(2-oxo-1-piperidyl)methyl]benzimidazol-1-yl]-2-pyridyl]-5-methyl-pyrazole-3-carbonitrile (30 mg, 0.070 mmol, 14.37% yield) and 1-[3-acetyl-6-[5-[(2-oxo-1-piperidyl)methyl]benzimidazol-1-yl]-2-pyridyl]-5-methyl-pyrazole-3-carbonitrile (30 mg, 0.070 mmol, 14.37% yield) as yellow oils after separation by preparative TLC (silica gel, 10% MeOH in DCM). Regioisomer 1: LC-MS: m/z=454.1 [M+H]⁺, ESI pos. ¹H NMR (400 MHz, DMSO-d₆) δ=9.15 (s, 1H), 8.56 (d, J=8.4 Hz, 1H), 8.31 (d, J=8.6 Hz, 1H), 8.18 (d, J=8.4 Hz, 1H), 7.65 (s, 1H), 7.31 (br d, J=8.8 Hz, 1H), 7.13 (s, 1H), 4.63 (s, 2H), 3.21 (br s, 2H), 2.52 (br s, 3H), 2.32 (br s, 2H), 2.21 (s, 3H), 1.71 (br s, 4H). Regioisomer 2: LC-MS: m/z=454.1 [M+H]⁺, ESI pos. ¹H NMR (400 MHz, DMSO-d₆) δ=9.13 (s, 1H), 8.57 (d, J=8.4 Hz, 1H), 8.30 (d, J=8.6 Hz, 1H), 8.05 (s, 1H), 7.76 (d, J=8.2 Hz, 1H), 7.27 (br d, J=8.2 Hz, 1H), 7.16 (s, 1H), 4.63 (s, 2H), 3.17 (br s, 2H), 2.48 (br s, 3H), 2.21 (s, 5H), 1.59 (br s, 4H).

### Step 4: 1-[3-(-hydroxyethyl)-6-[6-[(2-oxo-1-piperidyl)methyl]benzimidazol-1-yl]-2-pyridyl]-5-methyl-pyrazole-3-carbonitrile

Error! Objects cannot be created from editing field codes.

Prepared in analogy to example 53, step 4 using 1-[3-acetyl-6-[6-[(2-oxo-1-piperidyl)methyl]benzimidazol-1-yl]-2-pyridyl]-5-methyl-pyrazole-3-carbonitrile (30.0 mg, 0.070 mmol, 1.0 equiv.) and NaBH₄ (8.0 mg, 0.210 mmol, 3.2 equiv.) in MeOH/THF (1:3) to afford 1-[3-(1-hydroxyethyl)-6-[6-[(2-oxo-1-piperidyl)methyl]benzimidazol-1-yl]-2-pyridyl]-5-methyl-pyrazole-3-carbonitrile (5.2 mg, 0.010 mmol, 16.8% yield) as white solid. LC-MS: m/z=456.2 [M+H]⁺, ESI pos. ¹H NMR (400 MHz, DMSO-d₆) δ=9.04 (s, 1H), 8.46 (d, J=8.5 Hz, 1H), 8.26 (d, J=8.5 Hz, 1H), 7.95 (s, 1H), 7.73 (d, J=8.3 Hz, 1H), 7.23 (dd, J=1.5, 8.3 Hz, 1H), 7.12 (d, J=0.8 Hz, 1H), 5.54 (d, J=4.4 Hz, 1H), 4.61 (s, 2H), 4.55-4.47 (m, 1H), 3.15 (br t, J=5.6 Hz, 2H), 2.31 (s, 3H), 2.18 (t, J=6.3 Hz, 2H), 1.65-1.53 (m, 4H), 1.25 (d, J=6.5 Hz, 3H).

### Example 216

### 1-[3-(1-Hydroxyethyl)-6-[5-[(2-oxo-1-piperidyl)methyl]benzimidazol-1-yl]-2-pyridyl]-5-methyl-pyrazole-3-carbonitrile

Error! Objects cannot be created from editing field codes.

Prepared in analogy to example 53, step 4 using 1-[3-acetyl-6-[5-[(2-oxo-1-piperidyl)methyl]benzimidazol-1-yl]-2-pyridyl]-5-methyl-pyrazole-3-carbonitrile (30.0 mg, 0.070 mmol, 1.0 equiv., obtained in example 215, step 3) and NaBH4 (8.0 mg, 0.210 mmol, 3.2 equiv.) in MeOH/THF (1:3) to afford 1-[3-(1-hydroxyethyl)-6-[5-[(2-oxo-1-piperidyl)methyl]benzimidazol-1-yl]-2-pyridyl]-5-methyl-pyrazole-3-carbonitrile (6.5 mg, 0.010 mmol, 21.1% yield) as white solid. LC-MS: m/z=456.2 [M+H]⁺, ESI pos. ¹H NMR (400 MHz, DMSO-d₆) δ=9.04 (s, 1H), 8.46 (d, J=8.5 Hz, 1H), 8.27 (d, J=8.5 Hz, 1H), 8.07 (d, J=8.5 Hz, 1H), 7.63 (s, 1H), 7.27 (dd, J=1.3, 8.4 Hz, 1H), 7.09 (d, J=0.6 Hz, 1H), 5.64-5.46 (m, 1H), 4.62 (s, 2H), 4.56 (q, J=6.3 Hz, 1H), 3.20 (br s, 2H), 2.34 (s, 3H), 2.32 (br d, J=2.6 Hz, 2H), 1.70 (br t, J=3.3 Hz, 4H), 1.26 (d, J=6.5 Hz, 3H).

### Example 219

### 3-[[1-[6-(3-Cyano-5-methyl-pyrazol-1-yl)-5-(1-hydroxyethyl)-2-pyridyl]benzimidazol-5-yl]amino]-N,N,6-trimethyl-pyridazine-4-carboxamide

Error! Objects cannot be created from editing field codes.

### Step 1: 3-chloro-N,N,6-trimethyl-pyridazine-4-carboxamide

To a solution of 3-chloro-6-methylpyridazine-4-carboxylic acid (900.0 mg, 5.22 mmol, 1.0 equiv.) in DMF (10 mL) were added dimethylamine hydrochloride (510.3 mg, 6.26 mmol, 1.2 equiv.), DIPEA (2.73 mL, 15.65 mmol, 3 equiv.) and HATU (1.84 g, 7.82 mmol, 1.5 equiv.). The reaction mixture was stirred at 30° C. for 16 h. The reaction mixture was poured into water and extracted with EtOAc and DCM. The combined organic layers were dried over Na₂SO₄, and the volatiles evaporated. The crude product was purified by flash column chromatography (0-100% EtOAc in PE). Further purification by preparative HPLC (Phenomenex luna C18 150 mm×40 mm×15 μm, gradient 1-30% CH₃CN in H₂O (with 0.225% formic acid) over 13 min, then 100% CH₃CN (2 min), flow rate 60 mL/min) to give 3-chloro-N,N,6-trimethyl-pyridazine-4-carboxamide (350 mg, 1.75 mmol, 33.62% yield) as yellow solid. LC-MS: m/z=200.0 [M+H]⁺, ESI pos.

### Step 2: 3-amino-N,N,6-trimethyl-pyridazine-4-carboxamide

Error! Objects cannot be created from editing field codes.

A solution of 3-chloro-N,N,6-trimethyl-pyridazine-4-carboxamide (300.0 mg, 1.5 mmol, 1.0 equiv.) in 7M NH₃ in MeOH (8.0 mL, 56 mmol, 37 equiv.) in an autoclave was stirred at 120° C. for 72 h. The reaction mixture was concentrated under reduced pressure. The crude was purified by preparative HPLC (Waters Atlantis 150 mm×30 mm×5 μm, gradient 1-20% CH₃CN in H₂O (with 0.225% formic acid) over 10 min, then 100% CH₃CN (2 min), flow rate 25 mL/min). The resulting product was purified by preparative TLC (10% MeOH in DCM) to yield 3-amino-N,N,6-trimethyl-pyridazine-4-carboxamide (48 mg, 0.270 mmol, 17.4% yield) as an off-white solid. LC-MS: m/z=181.0 [M+H]⁺, ESI pos. ¹H NMR (400 MHz, CDCl₃) δ=6.92 (s, 1H), 5.24 (br s, 2H), 3.10-3.01 (m, 3H), 2.94 (br d, J=9.9 Hz, 3H), 2.51 (s, 3H).

### Step 3: 3-[[1-[5-acetyl-6-(3-cyano-5-methyl-pyrazol-1-yl)-2-pyridyl]benzimidazol-5-yl]amino]-N,N,6-trimethyl-pyridazine-4-carboxamide

Error! Objects cannot be created from editing field codes.

Prepared in analogy to example 104, step 2 using 1-[3-acetyl-6-(5-bromobenzimidazol-1-yl)-2-pyridyl]-5-methyl-pyrazole-3-carbonitrile (30 mg, 0.071 mmol, 1.0 equiv., prepared in example 65, step 1) and 3-amino-N,N,6-trimethyl-pyridazine-4-carboxamide (25.67 mg, 0.142 mmol, 2.0 equiv.) to yield the title compound (14.2 mg, 36.4% yield) as a yellow solid. LC-MS: m/z=521.3 [M+H]⁺, ESI pos.

### Step 4: 3-[[1-[6-(3-cyano-5-methyl-pyrazol-1-yl)-5-(1-hydroxyethyl)-2-pyridyl]benzimidazol-5-yl]amino]-N,N,6-trimethyl-pyridazine-4-carboxamide

Error! Objects cannot be created from editing field codes.

Prepared in analogy to example 53, step 4 using 3-[[1-[5-acetyl-6-(3-cyano-5-methyl-pyrazol-1-yl)-2-pyridyl]benzimidazol-5-yl]amino]-N,N,6-trimethyl-pyridazine-4-carboxamide (14.2 mg, 0.027 mmol, 1.0 equiv.) in MeOH/THF to yield the title compound (7.7 mg, 51.3% yield) as a light yellow solid. LC-MS: m/z=523.3 [M+H]⁺, ESI pos.

### Example 220

### 5-Hydroxy-2-[3-(1-hydroxyethyl)-6-[5-[(6-methylpyridazin-3-yl)amino]benzimidazol-1-yl]-2-pyridyl]benzonitrile; formic acid

Error! Objects cannot be created from editing field codes.

### Step 1: 2-[3-acetyl-6-[5-[(6-methylpyridazin-3-yl)amino]benzimidazol-1-yl]-2-pyridyl]-5-hydroxy-benzonitrile

Error! Objects cannot be created from editing field codes.

Prepared in analogy to example 143, step 2 using 1-[2-chloro-6-[5-[(6-methylpyridazin-3-yl)amino]benzimidazol-1-yl]-3-pyridyl]ethanone (50.0 mg, 0.130 mmol, 1.0 equiv., prepared in example 65, step 1) and (2-cyano-4-hydroxy-phenyl) (25.81 mg, 0.160 mmol, 1.2 equiv.) to yield 2-[3-acetyl-6-[5-[(6-methylpyridazin-3-yl)amino]benzimidazol-1-yl]-2-pyridyl]-5-hydroxy-benzonitrile (30 mg, 0.070 mmol, 49.3% yield) as a yellow solid LC-MS: m/z=462.2 [M+H]⁺, ESI pos.

### Step 2: 5-hydroxy-2-[3-(1-hydroxyethyl)-6-[5-[(6-methylpyridazin-3-yl)amino]benzimidazol-1-yl]-2-pyridyl]benzonitrile; formic acid

Error! Objects cannot be created from editing field codes.

Prepared in analogy to example 53, step 4 using 2-[3-acetyl-6-[5-[(6-methylpyridazin-3-yl)amino]benzimidazol-1-yl]-2-pyridyl]-5-hydroxy-benzonitrile (30.0 mg, 0.070 mmol, 1.0 equiv.) and NaBH₄ (12.36 mg, 0.330 mmol, 5.0 equiv.) to yield 5-hydroxy-2-[3-(1-hydroxyethyl)-6-[5-[(6-methylpyridazin-3-yl)amino]benzimidazol-1-yl]-2-pyridyl]benzonitrile; formic acid (4.5 mg, 0.010 mmol, 15.0% yield) as a white. LC-MS: m/z=464.1 [M+H]⁺, ESI pos. ¹H NMR (400 MHz, CD₃OD) δ=8.85 (s, 1H), 8.36 (d, J=8.4 Hz, 1H), 8.32-8.25 (m, 1H), 8.23-8.12 (m, 2H), 7.96 (d, J=8.4 Hz, 1H), 7.62-7.54 (m, 1H), 7.50 (d, J=8.4 Hz, 1H), 7.36 (d, J=9.2 Hz, 1H), 7.30 (d, J=2.4 Hz, 1H), 7.26-7.18 (m, 1H), 7.14 (d, J=9.2 Hz, 1H), 4.90 (s, 1H), 2.53 (s, 3H), 1.42 (d, J=6.4 Hz, 3H).

### Example 223

### 5-[3-(1-Hydroxyethyl)-6-[5-[(6-methylpyridazin-3-yl)amino]benzimidazol-1-yl]-2-pyridyl]-3-(trifluoromethyl)-1H-pyridazin-6-one; formic acid

Error! Objects cannot be created from editing field codes.

### Step 1: 1-[2-[3-methoxy-6-(trifluoromethyl)pyridazin-4-yl]-6-[5-[(6-methylpyridazin-3-yl)amino]benzimidazol-1-yl]-3-pyridyl]ethanone

Error! Objects cannot be created from editing field codes.

Prepared in analogy to example 143, step 2 using 1-[2-chloro-6-[5-[(6-methylpyridazin-3-yl)amino]benzimidazol-1-yl]-3-pyridyl]ethanone (80.0 mg, 0.210 mmol, 1.0 equiv., prepared in example 76, step 2) and 1-[2-chloro-6-[5-[(6-methylpyridazin-3-yl)amino]benzimidazol-1-yl]-3-pyridyl]ethanone (80.0 mg, 0.210 mmol, 1.0 equiv.) to yield 1-[2-[3-methoxy-6-(trifluoromethyl)pyridazin-4-yl]-6-[5-[(6-methylpyridazin-3-yl)amino]benzimidazol-1-yl]-3-pyridyl]ethanone (25 mg, 0.050 mmol, 22.8% yield) as yellow solid. LC-MS: m/z=521.3 [M+H]⁺, ESI pos.

### Step 2: 5-[3-acetyl-6-[5-[(6-methylpyridazin-3-yl)amino]benzimidazol-1-yl]-2-pyridyl]-3-(trifluoromethyl)-1H-pyridazin-6-one

Error! Objects cannot be created from editing field codes.

A solution of 1-[2-[3-methoxy-6-(trifluoromethyl)pyridazin-4-yl]-6-[5-[(6-methylpyridazin-3-yl)amino]benzimidazol-1-yl]-3-pyridyl]ethanone (20.0 mg, 0.040 mmol, 1.0 equiv.) in 37% aqueous HCl (0.16 mL, 2 mmol, 50 equiv.) was stirred at 100° C. for 3 h. The reaction mixture was concentrated under reduced pressure and purified by preparative HPLC (Phenomenex luna C18 (250×70 mm, 10 um), water (0.225% FA) —CH₃CN, 12-42% B, gradient time 20 min, 2 min 100% B hold time, flow rate 25 mL/min) to yield 5-[3-acetyl-6-[5-[(6-methylpyridazin-3-yl)amino]benzimidazol-1-yl]-2-pyridyl]-3-(trifluoromethyl)-1H-pyridazin-6-one (19 mg, 0.040 mmol, 97.6% yield) as a yellow solid. LC-MS: m/z=506.9 [M+H]⁺, ESI pos.

### Step 3: 5-[3-(1-hydroxyethyl)-6-[5-[(6-methylpyridazin-3-yl)amino]benzimidazol-1-yl]-2-pyridyl]-3-(trifluoromethyl)-1H-pyridazin-6-one; formic acid

Error! Objects cannot be created from editing field codes.

Prepared in analogy to example 53, step 4 using 5-[3-acetyl-6-[5-[(6-methylpyridazin-3-yl)amino]benzimidazol-1-yl]-2-pyridyl]-3-(trifluoromethyl)-1H-pyridazin-6-one (15.0 mg, 0.030 mmol, 1.0 equiv.) and NaBH₄ (5.63 mg, 0.150 mmol, 5.0 equiv.) to yield 5-[3-(1-hydroxyethyl)-6-[5-[(6-methylpyridazin-3-yl)amino]benzimidazol-1-yl]-2-pyridyl]-3-(trifluoromethyl)-1H-pyridazin-6-one; formic acid (3.3 mg, 0.010 mmol, 21.9% yield) as yellow solid. LC-MS: m/z=509.1 [M+H]⁺, ESI pos. ¹H NMR (400 MHz, CD₃OD) δ=8.85 (s, 1H), 8.32 (d, J=8.8 Hz, 1H), 8.26 (s, 1H), 8.23-8.09 (m, 2H), 8.05-7.95 (m, 2H), 7.62-7.52 (m, 1H), 7.36 (d, J=9.2 Hz, 1H), 7.14 (d, J=9.2 Hz, 1H), 4.89 (s, 1H), 2.53 (s, 3H), 1.49 (d, J=6.4 Hz, 3H).

### Example 226

### 2-[3-(1-Hydroxyethyl)-6-[5-[(6-methylpyridazin-3-yl)amino]benzimidazol-1-yl]-2-pyridyl]furan-3-carbonitrile

Error! Objects cannot be created from editing field codes.

### Step 1: 2-[3-acetyl-6-[5-[(6-methylpyridazin-3-yl)amino]benzimidazol-1-yl]-2-pyridyl]furan-3-carbonitrile

Error! Objects cannot be created from editing field codes.

Prepared in analogy to example 143, step 2 using (3-cyano-2-furyl)boronic acid (27.1 mg, 0.200 mmol, 1.5 equiv.) and 1-[2-chloro-6-[5-[(6-methylpyridazin-3-yl)amino]benzimidazol-1-yl]-3-pyridyl]ethanone (50.0 mg, 0.130 mmol, 1.0 equiv., prepared in example 64, step 1), to yield 2-[3-acetyl-6-[5-[(6-methylpyridazin-3-yl)amino]benzimidazol-1-yl]-2-pyridyl]furan-3-carbonitrile (20 mg, 0.050 mmol, 26.5% yield) as a yellow solid LC-MS: m/z=436.1, [M+H]⁺, ESI pos.

### Step 2: 2-[3-(1-hydroxyethyl)-6-[5-[(6-methylpyridazin-3-yl)amino]benzimidazol-1-yl]-2-pyridyl]furan-3-carbonitrile

Error! Objects cannot be created from editing field codes.

Prepared in analogy to example 53, step 4 using 2-[3-acetyl-6-[5-[(6-methylpyridazin-3-yl)amino]benzimidazol-1-yl]-2-pyridyl]furan-3-carbonitrile (20.0 mg, 0.050 mmol, 1.0 equiv.) and NaBH₄ (8.69 mg, 0.230 mmol, 5.0 equiv.) to yield 2-[3-(1-hydroxyethyl)-6-[5-[(6-methylpyridazin-3-yl)amino]benzimidazol-1-yl]-2-pyridyl]furan-3-carbonitrile (1 mg, 4.98% yield) as a white solid. LC-MS: m/z=438.2, [M+H]⁺, ESI pos. ¹H NMR (400 MHz, DMSO-d₆) δ=9.23 (s, 1H), 9.02 (s, 1H), 8.45-8.41 (m, 2H), 8.36 (s, 1H), 8.22-8.18 (m, 2H), 8.13 (d, J=8.6 Hz, 1H), 7.55 (dd, J=2.0, 8.8 Hz, 1H), 7.33 (d, J=9.2 Hz, 1H), 7.27 (d, J=2.0 Hz, 1H), 7.08 (d, J=9.2 Hz, 1H), 5.35 (q, J=6.4 Hz, 1H), 2.48 (s, 3H), 1.42 (d, J=6.4 Hz, 3H).

### Example 227

### 1-[2-[2-(2,2-Difluorocyclopropyl)pyrazol-3-yl]-6-[5-[(6-methylpyridazin-3-yl)amino]benzimidazol-1-yl]-3-pyridyl]ethanol; formic acid

Error! Objects cannot be created from editing field codes.

### Step 1: [2-(2,2-difluorocyclopropyl)pyrazol-3-yl]boronic acid

Error! Objects cannot be created from editing field codes.

Prepared in analogy to example 143, step 2 using 5-bromo-1-(2,2-difluorocyclopropyl)pyrazole (80.0 mg, 0.360 mmol, 1.0 equiv. to yield bis(pinacolato)diboron (136.64 mg, 0.540 mmol, 1.5 equiv.), potassium acetate (0.07 mL, 1.08 mmol, 3 equiv.) [2-(2,2-difluorocyclopropyl)pyrazol-3-yl]boronic acid which was used in the next step without purification. LC-MS: m/z=189.1, [M+H]⁺, ESI pos.

### Step 2: 1-[2-[2-(2,2-difluorocyclopropyl)pyrazol-3-yl]-6-[5-[(6-methylpyridazin-3-yl)amino]benzimidazol-1-yl]-3-pyridyl]ethanone

Error! Objects cannot be created from editing field codes.

Prepared in analogy to example 143, step 2 using 1-[2-chloro-6-[5-[(6-methylpyridazin-3-yl)amino]benzimidazol-1-yl]-3-pyridyl]ethanone (30.0 mg, 0.080 mmol, 1.0 equiv., prepared in example 76, step 2) and [2-(2,2-difluorocyclopropyl)pyrazol-3-yl]boronic acid (used as crude from the previous step), to yield 1-[2-[2-(2,2-difluorocyclopropyl)pyrazol-3-yl]-6-[5-[(6-methylpyridazin-3-yl)amino]benzimidazol-1-yl]-3-pyridyl]ethanone (17 mg, 0.030 mmol, 35.7% yield) as a yellow solid. LC-MS: m/z=487.1, [M+H]⁺, ESI pos.

### Step 3: 1-[2-[2-(2,2-difluorocyclopropyl)pyrazol-3-yl]-6-[5-[(6-methylpyridazin-3-yl)amino]benzimidazol-1-yl]-3-pyridyl]ethanol; formic acid

Error! Objects cannot be created from editing field codes.

Prepared in analogy to example 53, step 4 using 1-[2-[2-(2,2-difluorocyclopropyl)pyrazol-3-yl]-6-[5-[(6-methylpyridazin-3-yl)amino]benzimidazol-1-yl]-3-pyridyl]ethanone (15.0 mg, 0.030 mmol, 1.0 equiv.) and NaBH₄ (5.83 mg, 0.150 mmol, 5.0 equiv.) at −70° C. to yield 1-[2-[2-(2,2-difluorocyclopropyl)pyrazol-3-yl]-6-[5-[(6-methylpyridazin-3-yl)amino]benzimidazol-1-yl]-3-pyridyl]ethanol; formic acid (3.7 mg, 0.010 mmol, 22.5% yield) as a yellow solid. LC-MS: m/z=489.3, [M+H]⁺, ESI pos. ¹H NMR (400 MHz, CD₃OD) δ=8.88 (s, 1H), 8.41 (d, J=8.6 Hz, 1H), 8.24 (s, 1H), 8.20 (d, J=2.0 Hz, 1H), 8.09 (d, J=9.2 Hz, 1H), 8.00 (d, J=8.6 Hz, 1H), 7.69 (d, J=2.0 Hz, 1H), 7.59 (dd, J=2.0, 8.8 Hz, 1H), 7.37 (d, J=9.2 Hz, 1H), 7.15 (d, J=9.2 Hz, 1H), 6.68 (d, J=2.0 Hz, 1H), 5.07 (q, J=6.4 Hz, 1H), 4.47-4.38 (m, 1H), 2.53 (s, 3H), 2.23-2.05 (m, 2H), 1.50 (d, J=6.4 Hz, 3H).

### Example 228

### 1-[3-(1-Hydroxyethyl)-6-[5-[[6-(oxetan-3-yl)pyridazin-3-yl]amino]benzimidazol-1-yl]-2-pyridyl]-5-methyl-pyrazole-3-carbonitrile

Error! Objects cannot be created from editing field codes.

### Step 1:6-(oxetan-3-yl)pyridazin-3-amine; formic acid

Error! Objects cannot be created from editing field codes.

A 8 mL vial equipped with a stir bar was charged with 3-amino-6-bromopyridazine (1.0 g, 5.75 mmol, 1.0 equiv.), 3-iodooxetane (1.37 g, 7.47 mmol, 1.3 equiv.), Ir[dF(CF₃)ppy]2(dtbpy)(PF₆) (64.94 mg, 0.060 mmol, 0.01 equiv.), NiCl₂·dtbbpy (77.13 mg, 0.290 mmol, 0.050 equiv.), tris(trimethylsilyl)silane (1.43 g, 5.75 mmol, 1.0 equiv.) and Na₂CO₃ (1.22 g, 11.49 mmol, 2.0 equiv.) in DME (80 mL). The vial was sealed and placed under nitrogen. The reaction was stirred and irradiated with a 34 W blue LED lamp (distanced at 7 cm), in presence of a cooling fan to maintain the reaction at 25° C. for 14 h. The reaction mixture was quenched with water and extracted with ethyl acetate. And the combined aqueous phase was concentrated under reduced pressure to give a residue. The residue was preparative HPLC (Waters Atlantis T3 150 mm×30 mm×5 μm, gradient 1-20% CH₃CN in H₂O (with 0.225% formic acid) over 108 min, then 100% CH₃CN (2 min), flow rate 25 mL/min) to give 6-(oxetan-3-yl)pyridazin-3-amine; formic acid (65.8 mg, 0.330 mmol, 5.8% yield) as brown solid. LC-MS: m/z=152.0 [M+H]⁺, ESI pos. ¹H NMR (400 MHz, DMSO-d₆) δ=8.13 (s, 1H), 7.43 (d, J=9.1 Hz, 1H), 6.93 (d, J=9.1 Hz, 1H), 6.75 (br s, 2H), 4.88 (dd, J=5.9, 8.4 Hz, 2H), 4.72 (t, J=6.2 Hz, 2H), 4.33 (td, J=7.6, 15.1 Hz, 1H).

### Step 2: 1-[3-acetyl-6-[5-[[6-(oxetan-3-yl)pyridazin-3-yl]amino]benzimidazol-1-yl]-2-pyridyl]-5-methyl-pyrazole-3-carbonitrile

Error! Objects cannot be created from editing field codes.

Prepared in analogy to example 104, step 2 using 1-[3-acetyl-6-(5-bromobenzimidazol-1-yl)-2-pyridyl]-5-methyl-pyrazole-3-carbonitrile (30 mg, 0.071 mmol, 1 equiv., prepared in example 65, step 1) and [6-(oxetan-3-yl)pyridazin-3-yl]amine; formic acid (28.1 mg, 0.142 mmol, 2.0 equiv.) to yield the title compound (14.8 mg, 40.2% yield) as a yellow solid. LC-MS: m/z=492.3 [M+H]⁺, ESI pos.

### Step 3: 1-[3-(-hydroxyethyl)-6-[5-[[6-(oxetan-3-yl)pyridazin-3-yl]amino]benzimidazol-1-yl]-2-pyridyl]-5-methyl-pyrazole-3-carbonitrile

Error! Objects cannot be created from editing field codes.

Prepared in analogy to example 53, step 1 using 1-[3-acetyl-6-[5-[[6-(oxetan-3-yl)pyridazin-3-yl]amino]benzimidazol-1-yl]-2-pyridyl]-5-methyl-pyrazole-3-carbonitrile (14.8 mg, 0.029 mmol, 1.0 equiv.) in MeOH/THF (1:1) to yield the title compound (7.7 mg, 53.8% yield) as a light yellow solid. LC-MS: m/z=494.3 [M+H]⁺, ESI pos.

### Example 229

### (1S)-1-[2-[3-(Difluoromethyl)-5-methyl-pyrazol-1-yl]-6-[5-[(6-methylpyridazin-3-yl)amino]benzimidazol-1-yl]-3-pyridyl]ethanol

Error! Objects cannot be created from editing field codes.

The enantiomers of 1-[2-[3-(difluoromethyl)-5-methyl-pyrazol-1-yl]-6-[5-[(6-methylpyridazin-3-yl)amino]benzimidazol-1-yl]-3-pyridyl]ethanol (121.5 mg, 0.255 mmol, 1 equiv., from example 129) were separated by chiral SFC (chiral OJ-H 250 mm×20 mm×5 μm, 18% MeOH (with 0.2% diethylamine)) to yield (1S)-1-[2-[3-(difluoromethyl)-5-methyl-pyrazol-1-yl]-6-[5-[(6-methylpyridazin-3-yl)amino]benzimidazol-1-yl]-3-pyridyl]ethanol (43 mg, 34.3%, 95.6% ee) as off-white lyophilized solid. Assignment of the absolute stereochemistry was done in analogy to examples 109 and 110.

### Example 230

### (1R)-1-[2-[3-(Difluoromethyl)-5-methyl-pyrazol-1-yl]-6-[5-[(6-methylpyridazin-3-yl)amino]benzimidazol-1-yl]-3-pyridyl]ethanol

Error! Objects cannot be created from editing field codes.

The enantiomers of 1-[2-[3-(difluoromethyl)-5-methyl-pyrazol-1-yl]-6-[5-[(6-methylpyridazin-3-yl)amino]benzimidazol-1-yl]-3-pyridyl]ethanol (121.5 mg, 0.255 mmol, 1 equiv., from example 129) were separated by chiral FC (chiral OJ-H 250 mm×20 mm×5 μm, 18% MeOH (with 0.2% diethylamine)) to yield (1R)-1-[2-[3-(difluoromethyl)-5-methyl-pyrazol-1-yl]-6-[5-[(6-methylpyridazin-3-yl)amino]benzimidazol-1-yl]-3-pyridyl]ethanol (50 mg, 41.2%, 100% ee). Assignment of the absolute stereochemistry was done in analogy to examples 109 and 110.

### Example 231

### 1-[2-[5-(difluoromethyl)-3-methyl-pyrazol-1-yl]-6-[5-[(6-methylpyridazin-3-yl)amino]benzimidazol-1-yl]-3-pyridyl]ethanol

Was obtained in example 129 from the reduction of the trace mounts of 1-[2-[5-(difluoromethyl)-3-methyl-pyrazol-1-yl]-6-[5-[(6-methylpyridazin-3-yl)amino]benzimidazol-1-yl]-3-pyridyl]ethanone contained in 1-[2-[3-(difluoromethyl)-5-methyl-pyrazol-1-yl]-6-[5-[(6-methylpyridazin-3-yl)amino]benzimidazol-1-yl]-3-pyridyl]ethanone to give 1-[2-[5-(difluoromethyl)-3-methyl-pyrazol-1-yl]-6-[5-[(6-methylpyridazin-3-yl)amino]benzimidazol-1-yl]-3-pyridyl]ethanol (3.4 mg, 2.24% yield) as a white solid after purification SFC (chiral OJ-H 250 mm×20 mm×5 μm, 18% MeOH (with 0.2% diethylamine)). LC-MS: m/z=477.2 [M+H]⁺, ESI pos. ¹H NMR (600 MHz, CDCl₃) δ=8.54 (s, 1H), 8.27 (d, J=8.3 Hz, 1H), 7.89 (d, J=8.7 Hz, 1H), 7.78 (d, J=1.9 Hz, 1H), 7.62 (d, J=8.2 Hz, 1H), 7.38 (dd, J=8.7, 2.1 Hz, 1H), 7.24-7.26 (m, 1H), 7.12-7.15 (m, 1H), 7.01-7.09 (m, 1H), 6.97-7.05 (m, 1H), 6.67-6.68 (m, 1H), 5.05-5.20 (m, 1H), 4.99 (q, J=6.5 Hz, 1H), 2.59-2.61 (m, 4H), 2.43 (s, 3H), 1.57-1.58 (m, 10H).

### Example 234

### 1-[3-(1-Hydroxy-2-methoxy-ethyl)-6-[5-[(6-methylpyridazin-3-yl)amino]benzimidazol-1-yl]-2-pyridyl]-5-methyl-pyrazole-3-carbonitrile; formic acid

Error! Objects cannot be created from editing field codes.

### Step 1: 1-[6-chloro-3-(2-methoxyacetyl)-2-pyridyl]-5-methyl-pyrazole-3-carbonitrile

Error! Objects cannot be created from editing field codes.

A mixture of 5-methyl-1H-pyrazole-3-carbonitrile (315.7 mg, 2.95 mmol, 1.0 equiv.), DIPEA (1.0 mL, 5.89 mmol, 2.0 equiv.) and 1-(6-chloro-2-fluoro-3-pyridyl)-2-methoxy-ethanone (600.0 mg, 2.95 mmol, 1.0 equiv.) in DMSO (10 mL) was stirred at 100° C. for 3 h. The reaction mixture poured into water and extracted with EtOAc, the combined organic layers were dried and concentrated under vacuum. The combined residue was purified by flash column chromatography (silica gel, 25% EtOAc, in PE) to give 1-[6-chloro-3-(2-methoxyacetyl)-2-pyridyl]-5-methyl-pyrazole-3-carbonitrile (500 mg, 1.72 mmol, 58.4% yield) as a yellow gum. ¹H NMR (400 MHz, CDCl₃) δ=7.91 (d, J=8.0 Hz, 1H), 7.48 (d, J=8.0 Hz, 1H), 6.63 (s, 1H), 4.05 (s, 2H), 3.19 (s, 3H), 2.66 (s, 3H).

### Step 2: 1-[3-(2-methoxyacetyl)-6-[5-[(6-methylpyridazin-3-yl)amino]benzimidazol-1-yl]-2-pyridyl]-5-methyl-pyrazole-3-carbonitrile and 1-[3-(2-methoxyacetyl)-6-[6-[(6-methylpyridazin-3-yl)amino]benzimidazol-1-yl]-2-pyridyl]-5-methyl-pyrazole-3-carbonitrile

Error! Objects cannot be created from editing field codes.

Prepared in analogy to example 76, step 1 using 1-[6-chloro-3-(2-methoxyacetyl)-2-pyridyl]-5-methyl-pyrazole-3-carbonitrile (500.0 mg, 1.72 mmol, 1.0 equiv.), DIPEA (0.6 mL, 3.44 mmol, 2 equiv.) and N— (6-methylpyridazin-3-yl)-1H-benzimidazol-5-amine (387.41 mg, 1.72 mmol, 1.0 equiv., prepared in example 64, intermediate 1) to yield 1-[3-(2-methoxyacetyl)-6-[5-[(6-methylpyridazin-3-yl)amino]benzimidazol-1-yl]-2-pyridyl]-5-methyl-pyrazole-3-carbonitrile (30 mg, 0.060 mmol, 3.6% yield) and 1-[3-(2-methoxyacetyl)-6-[6-[(6-methylpyridazin-3-yl)amino]benzimidazol-1-yl]-2-pyridyl]-5-methyl-pyrazole-3-carbonitrile (20 mg, 0.040 mmol, 2.4% yield) as two grey solid after separation by preparative HPLC (Phenomenex luna C18 150 mm×25 mm×10 μm, gradient 14-34% CH₃CN in H₂O (with 0.225% formic acid) over 10 min, then 100% CH₃CN (5 min), flow rate 25 mL/min) and preparative NPLC (Welch Ultimate XB-SiOH 250 mm×50 mm×10 μm, gradient 29-45% EtOH (with 0.1% ammonium hydroxide) in hexane over 15 min, then 100% EtOH (with 0.1% ammonium hydroxide) (5 min), flow rate 100 mL/min, 1 injection). LC-MS: m/z=480.3 [M+H]⁺, ESI pos.

### Step 3: 1-[3-(-hydroxy-2-methoxy-ethyl)-6-[5-[(6-methylpyridazin-3-yl)amino]benzimidazol-1-yl]-2-pyridyl]-5-methyl-pyrazole-3-carbonitrile; formic acid

Error! Objects cannot be created from editing field codes.

Prepared in analogy to example 53, step 4 using 1-[3-(2-methoxyacetyl)-6-[5-[(6-methylpyridazin-3-yl)amino]benzimidazol-1-yl]-2-pyridyl]-5-methyl-pyrazole-3-carbonitrile (30.0 mg, 0.060 mmol, 1.0 equiv.) to give 1-[3-(1-hydroxy-2-methoxy-ethyl)-6-[5-[(6-methylpyridazin-3-yl)amino]benzimidazol-1-yl]-2-pyridyl]-5-methyl-pyrazole-3-carbonitrile; formic acid (13.6 mg, 0.030 mmol, 45.1% yield) as a yellow soli. LC-MS: m/z=482.2 [M+H]⁺, ESI pos. ¹H NMR (400 MHz, DMSO-d₆) δ=8.94 (s, 1H), 8.40 (d, J=8.6 Hz, 1H), 8.35 (s, 1H), 8.17 (d, J=8.7 Hz, 1H), 8.13-8.10 (m, 1H), 8.03 (d, J=8.8 Hz, 1H), 7.51-7.46 (m, 1H), 7.37 (d, J=9.2 Hz, 1H), 7.12 (d, J=9.2 Hz, 1H), 7.02 (s, 1H), 4.60 (t, J=5.5 Hz, 1H), 3.36 (t, J=5.1 Hz, 2H), 3.14 (s, 3H), 2.46 (s, 3H), 2.32 (s, 3H).

### Example 235

### 1-[3-(1-Hydroxy-2-methoxy-ethyl)-6-[6-[(6-methylpyridazin-3-yl)amino]benzimidazol-1-yl]-2-pyridyl]-5-methyl-pyrazole-3-carbonitrile

Error! Objects cannot be created from editing field codes.

Prepared in analogy to example 53, step 4 using 1-[3-(2-methoxyacetyl)-6-[6-[(6-methylpyridazin-3-yl)amino]benzimidazol-1-yl]-2-pyridyl]-5-methyl-pyrazole-3-carbonitrile (20.0 mg, 0.040 mmol, 1.0 equiv., prepared in example 234, step 2) to give 1-[3-(1-hydroxy-2-methoxy-ethyl)-6-[6-[(6-methylpyridazin-3-yl)amino]benzimidazol-1-yl]-2-pyridyl]-5-methyl-pyrazole-3-carbonitrile (4.4 mg, 0.010 mmol, 21.9% yield) as a yellow solid. LC-MS: m/z=482.2 [M+H]⁺, ESI pos. ¹H NMR (400 MHz, CD₃OD) δ=8.80 (s, 1H), 8.76 (d, J=1.8 Hz, 1H), 8.55 (d, J=8.6 Hz, 1H), 8.15 (d, J=8.3 Hz, 1H), 7.69 (d, J=8.8 Hz, 1H), 7.45 (dd, J=2.0, 8.7 Hz, 1H), 7.36 (d, J=9.2 Hz, 1H), 7.15 (d, J=9.2 Hz, 1H), 6.81 (s, 1H), 4.68-4.61 (m, 1H), 3.58-3.48 (m, 2H), 3.30 (br s, 3H), 2.56 (s, 3H), 2.39 (s, 3H).

### Example 236

### 1-[6-[6-Fluoro-5-[(6-methylpyridazin-3-yl)amino]benzimidazol-1-yl]-3-[(1S)-1-hydroxyethyl]-2-pyridyl]-5-methyl-pyrazole-3-carbonitrile

Error! Objects cannot be created from editing field codes.

The two enantiomers of 1-[6-[6-fluoro-5-[(6-methylpyridazin-3-yl)amino]benzimidazol-1-yl]-3-(1-hydroxyethyl)-2-pyridyl]-5-methyl-pyrazole-3-carbonitrile (120.0 mg, from Example 93/94, step 6) were separated by chiral SFC (Daicel Chiralpak AS 250 mm×30 mm×10 μm, 35% MeOH (with 0.1% ammonium hydroxide) over 30 min, 65 mL/min flow rate) to yield 1-[6-[6-fluoro-5-[(6-methylpyridazin-3-yl)amino]benzimidazol-1-yl]-3-[(1S)-1-hydroxyethyl]-2-pyridyl]-5-methyl-pyrazole-3-carbonitrile (44 mg, 0.090 mmol, 36.67% yield) as light yellow solid. (S)-isomer: LC-MS: m/z=470.0 [M+H]⁺, ESI pos. ¹H NMR (400 MHz, CD₃OD) δ=8.94 (s, 1H), 8.54 (dd, J=7.9, 15.9 Hz, 2H), 8.17 (d, J=8.6 Hz, 1H), 8.01 (d, J=11.2 Hz, 1H), 7.40 (d, J=9.2 Hz, 1H), 7.22 (d, J=9.0 Hz, 1H), 6.90 (s, 1H), 4.78 (q, J=6.3 Hz, 1H), 2.56 (s, 3H), 2.43 (s, 3H), 1.43 (d, J=6.5 Hz, 3H). Assignment of the absolute stereochemistry was done in analogy to examples 109 and 110.

### Example 237

### 1-[6-[6-Fluoro-5-[(6-methylpyridazin-3-yl)amino]benzimidazol-1-yl]-3-[(1R)-1-hydroxyethyl]-2-pyridyl]-5-methyl-pyrazole-3-carbonitrile

Error! Objects cannot be created from editing field codes.

1-[6-[6-fluoro-5-[(6-methylpyridazin-3-yl)amino]benzimidazol-1-yl]-3-[(1R)-1-hydroxyethyl]-2-pyridyl]-5-methyl-pyrazole-3-carbonitrile (50 mg, 0.110 mmol, 41.7% yield) was obtained as white solid after separation of both enantiomers by chiral SFC (Daicel Chiralpak AS 250 mm×30 mm×10 μm, 35% MeOH (with 0.1% ammonium hydroxide) over 30 min, 65 mL/min flow rate). (R)-isomer: LC-MS: m/z=470.0 [M+H]⁺, ESI pos. ¹H NMR (400 MHz, CD₃OD) δ=8.94 (s, 1H), 8.54 (dd, J=7.9, 13.8 Hz, 2H), 8.17 (d, J=8.6 Hz, 1H), 8.01 (d, J=11.4 Hz, 1H), 7.41 (d, J=9.2 Hz, 1H), 7.23 (d, J=9.2 Hz, 1H), 6.90 (s, 1H), 4.78 (q, J=6.4 Hz, 1H), 2.56 (s, 3H), 2.43 (s, 3H), 1.43 (d, J=6.4 Hz, 3H). Assignment of the absolute stereochemistry was done in analogy to examples 109 and 110.

### Example 239

### 1-[5-Chloro-3-(1-hydroxyethyl)-6-[5-[(6-methylpyridazin-3-yl)amino]benzimidazol-1-yl]-2-pyridyl]pyrazole-3-carbonitrile

Error! Objects cannot be created from editing field codes.

### Step 1: 2,5,6-trichloropyridine-3-carbonyl chloride

Error! Objects cannot be created from editing field codes.

To a suspension of 2,5,6-trichloronicotinic acid (5.0 g, 22.08 mmol, 1.0 equiv.) in DCM (50 mL) were added dropwise oxalyl chloride (4.3 mL, 50.79 mmol, 2.3 equiv.) and DMF (161.39 mg, 2.21 mmol, 0.10 equiv.) at 0° C. The reaction mixture was stirred at RT for 30 minutes. The resulting clear solution was concentrated to dryness yield 2,5,6-trichloropyridine-3-carbonyl chloride as light yellow oil which was used in the ext step without further purification. LC-MS: m/z=239.9 [M+H]⁺, ESI pos.

### Step 2: 2,5,6-trichloro-N-methoxy-N-methyl-pyridine-3-carboxamide

Error! Objects cannot be created from editing field codes.

To a suspension of 2,5,6-trichloropyridine-3-carbonyl chloride (used as crude from the previous step) in DCM (54 mL) was added TEA (6.69 g, 66.15 mmol, 3.0 equiv.). After 3 min, O,N-dimethylhydroxylamine HCl (2.37 g, 24.26 mmol, 1.1 equiv.) in DCM (10 mL) was added dropwise to the solution at 0° C. The reaction mixture was stirred at RT for 1 hour during which a yellow precipitate formed. The reaction mixture was quenched with water and extracted with DCM. The combined organic layers were dried over Na₂SO₄ and concentrated under reduced pressure. The residue was purified by flash column chromatography (silica gel, 40% EtOAc in PE) to yield 2,5,6-trichloro-N-methoxy-N— methyl-pyridine-3-carboxamide (5.7 g, 21.15 mmol, 95.9% yield) as off-white solid. LC-MS: m/z=269.0 [M+H]⁺, ESI pos.

### Step 3: 1-(2,5,6-trichloro-3-pyridyl)ethanone

Error! Objects cannot be created from editing field codes.

2,5,6-trichloro-N-methoxy-N-methyl-pyridine-3-carboxamide (5.7 g, 21.15 mmol, 1.0 equiv.) was dissolved in THF (60 mL). MeMgBr (21.2 mL, 63.45 mmol, 3.0 equiv.) was added dropwise at 0° C. under nitrogen atmosphere. After several minutes, an orange solid recipitated. The mixture was stirred for another 2 h at 0° C. The reaction mixture was then quenched with water and extracted with EtOAc.

The combined organic layers were dried over Na₂SO₄ and concentrated under reduced pressure. The residue was purified by flash column chromatography (silica gel, 16-25% EtOAc in PE) to yield 1-(2,5,6-trichloro-3-pyridyl)ethanone (4.5 g, 20.05 mmol, 94.8% yield) as colorless oil. LC-MS: m/z=223.9 [M−H]⁻, ESI neg. ¹H NMR (400 MHz, CDCl₃) δ=8.05 (s, 1H), 2.73 (s, 3H).

### Step 4: 1-[2,5-dichloro-6-[5-[(6-methylpyridazin-3-yl)amino]benzimidazol-1-yl]-3-pyridyl]ethanone

Error! Objects cannot be created from editing field codes.

To a solution of 1-(2,5,6-trichloro-3-pyridyl)ethanone (2.0 g, 8.91 mmol, 1.0 equiv.) in DMSO (20 mL) were added N-(6-methylpyridazin-3-yl)-1H-benzimidazol-5-amine (2.01 g, 8.91 mmol, 1.0 equiv., prepared in example 64, intermediate 1) and DIPEA (3.11 mL, 17.82 mmol, 2.0 equiv.). The reaction mixture was stirred at 100° C. for 16 h. The reaction mixture was added into water which lead to the formation of a black precipitate The filter cake was purified by flash column chromatography (silica gel, 10% MeOH in DCM) which led to the separation of the two regioisomeric products. Further purification by preparative NPLC (Welch Ultimate XB-SiOH 250 mm×70 mm×10 μm, gradient 25-65% EtOH (with 0.1% ammonium hydroxide) in heptane over 15 min, then 100% EtOH (with 0.1% ammonium hydroxide) (3 min), flow rate 140 mL/min) to give 1-[2,5-dichloro-6-[5-[(6-methylpyridazin-3-yl)amino]benzimidazol-1-yl]-3-pyridyl]ethanone (800 mg, 1.94 mmol, 21.7% yield) as a brown gum. LC-MS: m/z=413.2 [M+H]⁺, ESI pos. ¹H NMR (400 MHz, DMSO-d₆) δ=8.99 (s, 1H), 8.51 (s, 1H), 8.45 (s, 1H), 8.20 (d, J=1.8 Hz, 1H), 7.36-7.32 (m, 1H), 7.29-7.25 (m, 1H), 7.10 (d, J=9.0 Hz, 1H), 6.85 (d, J=9.1 Hz, 1H), 2.48 (s, 3H), 2.25 (s, 3H).

### Step 5: 1-[3-acetyl-5-chloro-6-[5-[(6-methylpyridazin-3-yl)amino]benzimidazol-1-yl]-2-pyridyl]pyrazole-3-carbonitrile

Error! Objects cannot be created from editing field codes.

A solution of 1-[2,5-dichloro-6-[5-[(6-methylpyridazin-3-yl)amino]benzimidazol-1-yl]-3-pyridyl]ethanone (100.0 mg, 0.240 mmol, 1.0 equiv.), 1H-pyrazole-3-carbonitrile (22.53 mg, 0.240 mmol, 1.0 equiv.) and DIPEA (62.55 mg, 0.480 mmol, 2 equiv.) in DMSO (2 mL) was stirred at 100° C. for 16 h. The reaction mixture was poured into water under stirring, and a precipitate formed. The reaction mixture was filtered, the filter cake was dried under reduced pressure to give 1-[3-acetyl-5-chloro-6-[5-[(6-methylpyridazin-3-yl)amino]benzimidazol-1-yl]-2-pyridyl]pyrazole-3-carbonitrile (80 mg, 0.170 mmol, 70.4% yield) as yellow solid. LC-MS: m/z=470.2 [M+H]⁺, ESI pos. ¹H NMR (400 MHz, DMSO-d₆) δ=9.21 (s, 1H), 8.79 (d, J=2.7 Hz, 1H), 8.72 (s, 1H), 8.69 (s, 1H), 8.42 (d, J=1.8 Hz, 1H), 7.65 (d, J=8.8 Hz, 1H), 7.52 (dd, J=2.0, 8.8 Hz, 1H), 7.35-7.31 (m, 2H), 7.08 (d, J=9.0 Hz, 1H), 2.48 (s, 6H).

### Step 6: 1-[5-chloro-3-(-hydroxyethyl)-6-[5-[(6-methylpyridazin-3-yl)amino]benzimidazol-1-yl]-2-pyridyl]pyrazole-3-carbonitrile

Error! Objects cannot be created from editing field codes.

Prepared according to example 53, step 4 using 1-[3-acetyl-5-chloro-6-[5-[(6-methylpyridazin-3-yl)amino]benzimidazol-1-yl]-2-pyridyl]pyrazole-3-carbonitrile (100.0 mg, 0.210 mmol, 1.0 equiv.) and NaBH₄ (25.0 mg, 0.660 mmol, 3.1 equiv.) in MeOH/DCM (1:1) to afford 1-[5-chloro-3-(1-hydroxyethyl)-6-[5-[(6-methylpyridazin-3-yl)amino]benzimidazol-1-yl]-2-pyridyl]pyrazole-3-carbonitrile (29 mg, 0.060 mmol, 28% yield) as white solid. LC-MS: m/z=472.1[M+H]⁺, ESI pos. ¹H NMR (400 MHz, DMSO-d₆) δ=9.18 (s, 1H), 8.72-8.67 (m, 2H), 8.62 (s, 1H), 8.40 (s, 1H), 7.59-7.54 (m, 1H), 7.52-7.47 (m, 1H), 7.36-7.30 (m, 2H), 7.07 (d, J=9.0 Hz, 1H), 5.77-5.74 (m, 1H), 5.35-5.25 (m, 1H), 2.48 (br s, 3H), 1.37 (d, J=6.4 Hz, 3H).

### Example 240

### 1-[6-[5-[(6-Methylpyridazin-3-yl)amino]benzimidazol-1-yl]-2-[5-methyl-1-(2,2,2-trifluoroethyl)pyrazol-4-yl]-3-pyridyl]ethanol; formic acid

Error! Objects cannot be created from editing field codes.

### Step 1: 1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanone

Error! Objects cannot be created from editing field codes.

Prepared in analogy to example 143, step 2 using 3-methyl-4-(4,4,5,5-tetramethyl-1,3,2-dioxaborolan-2-yl)-1-(2,2,2-trifluoroethyl)pyrazole (574.33 mg, 1.98 mmol, 1.5 equiv., prepared in example 143, step 2) and 1-[2-chloro-6-[5-[(6-methylpyridazin-3-yl)amino]benzimidazol-1-yl]-3-pyridyl]ethanone (500.0 mg, 1.32 mmol, 1.0 equiv., prepared in example 76, step 2) to yield 1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanone (200 mg, 0.39 mmol, 29.9% yield) as yellow solid. LC-MS: m/z=507.1 [M+H]⁺, ESI pos. Separation by SFC (Chiralpak AD-3 50 mm×4.6 mm, 3 μm, 40% ⁱPrOH+CH₃CN (with 0.05% Et₂NH) in scCO₂, flow rate 3 mL/min, back pressure 100 bar) yielded 1-[6-[5-[(6-methylpyridazin-3-yl)amino]benzimidazol-1-yl]-2-[5-methyl-1-(2,2,2-trifluoroethyl)pyrazol-4-yl]-3-pyridyl]ethanone (650 mg, 1.28 mmol, 28.5% yield) as yellow solid. LC-MS: m/z=507.1 [M+H]⁺, ESI pos. ¹H NMR (400 MHz, CD₃OD) δ=8.92 (s, 1H), 8.39-8.17 (m, 3H), 7.97 (s, 1H), 7.87 (d, J=8.4 Hz, 1H), 7.68-7.57 (m, 1H), 7.35 (d, J=9.2 Hz, 1H), 7.13 (d, J=9.2 Hz, 1H), 5.01-4.94 (m, 2H), 2.53 (s, 3H), 2.39 (s, 3H), 2.36 (s, 3H).

### Step 2: 1-[6-[5-[(6-methylpyridazin-3-yl)amino]benzimidazol-1-yl]-2-[5-methyl-1-(2,2,2-trifluoroethyl)pyrazol-4-yl]-3-pyridyl]ethanol; formic acid

Error! Objects cannot be created from editing field codes.

Prepared in analogy to example 53, step 4 using 1-[6-[5-[(6-methylpyridazin-3-yl)amino]benzimidazol-1-yl]-2-[5-methyl-1-(2,2,2-trifluoroethyl)pyrazol-4-yl]-3-pyridyl]ethanone (30.0 mg, 0.060 mmol, 1.0 equiv.) and NaBH₄ (11.26 mg, 0.300 mmol, 5.0 equiv.) at −70° to yield formic acid; 1-[6-[5-[(6-methylpyridazin-3-yl)amino]benzimidazol-1-yl]-2-[5-methyl-1-(2,2,2-trifluoroethyl)pyrazol-4-yl]-3-pyridyl]ethanol (11.9 mg, 0.020 mmol, 38.09% yield) as yellow solid. LC-MS: m/z=509.1 [M+H]⁺, ESI pos. ¹H NMR (400 MHz, CD₃OD) δ=8.83 (s, 1H), 8.30 (d, J=8.4 Hz, 1H), 8.22-8.14 (m, 2H), 8.14 (s, 1H), 7.84 (d, J=8.4 Hz, 1H), 7.81 (s, 1H), 7.63-7.51 (m, 1H), 7.38 (d, J=9.2 Hz, 1H), 7.16 (d, J=9.2 Hz, 1H), 5.09-5.02 (m, 2H), 2.53 (s, 3H), 2.45 (s, 3H), 1.46 (d, J=6.4 Hz, 3H).

### Example 241

### 1-[6-[5-[(6-Chloro-4-methoxy-pyridazin-3-yl)amino]benzimidazol-1-yl]-3-(1-hydroxyethyl)-2-pyridyl]-5-methyl-pyrazole-3-carbonitrile

Error! Objects cannot be created from editing field codes.

### Step 1: 6-chloro-4-methoxypyridazin-3-amine

Error! Objects cannot be created from editing field codes.

To a stirred, brown slurry of 4-bromo-6-chloropyridazin-3-amine (6.888 g, 33.0 mmol, 1.0 equiv.) in MeOH (100 ml) under an argon atmosphere was added dropwise a 5.4M solution NaOMe in MeOH (7.34 ml, 39.7 mmol, 1.2 equiv.) diluted in MeOH (65 ml). Upon completion of the addition, stirring at RT was continued overnight. The dark brown slurry was concentrated and the residual dark brown solid was purified by flash column chromatography (silica gel, 0-5% MeOH in DCM) to yield the title compound (2.91 g, 55.1% yield) as a brown solid. LC-MS: m/z=160.1 [M+H]⁺, ESI pos.

### Step 2: 1-[3-acetyl-6-[5-[(6-chloro-4-methoxy-pyridazin-3-yl)amino]benzimidazol-1-yl]-2-pyridyl]-5-methyl-pyrazole-3-carbonitrile

Error! Objects cannot be created from editing field codes.

Prepared in analogy to example 104, step 2 using 1-[3-acetyl-6-(5-bromobenzimidazol-1-yl)-2-pyridyl]-5-methyl-pyrazole-3-carbonitrile (30 mg, 0.071 mmol, 1 equiv., prepared in example 65, step 1), (6-chloro-4-methoxy-pyridazin-3-yl)amine (22.73 mg, 0.142 mmol, 2.0 equiv.) and two additions of [tBuBrettPhos Pd(allyl)]OTf (5.56 mg, 0.007 mmol, 0.100 equiv.) in an interval of 2 h to yield the title compound (15.9 mg, 35.7% yield) as a yellow solid. LC-MS: m/z=492.3 [M+H]⁺, ESI pos.

### Step 3: 1-[6-[5-[(6-chloro-4-methoxy-pyridazin-3-yl)amino]benzimidazol-1-yl]-3-(-hydroxyethyl)-2-pyridyl]-5-methyl-pyrazole-3-carbonitrile

Error! Objects cannot be created from editing field codes.

Prepared in analogy to example 53, step 4 using 1-[3-acetyl-6-[5-[(6-chloro-4-methoxy-pyridazin-3-yl)amino]benzimidazol-1-yl]-2-pyridyl]-5-methyl-pyrazole-3-carbonitrile (17.3 mg, 0.033 mmol, 1 equiv.) and NaBH₄ (7.5 mg, 0.2 mmol, 6.0 equiv.) in MeOH/THF (1:1) to yield the title compound (9 mg, 52.9% yield) as a light yellow solid. LC-MS: m/z=502.3 [M+H]⁺, ESI pos.

### Example 242

### (3R,5S)-1-[3-(1-hydroxyethyl)-6-[5-[(6-methylpyridazin-3-yl)amino]benzimidazol-1-yl]-2-pyridyl]-5-methyl-pyrrolidine-3-carbonitrile; formic acid

Error! Objects cannot be created from editing field codes.

### Step 1: tert-butyl (2S,4S)-2-methyl-4-methylsulfonyloxy-pyrrolidine-1-carboxylate

Error! Objects cannot be created from editing field codes.

To a solution of tert-butyl (2R,4R)-4-hydroxy-2-methyl-pyrrolidine-1-carboxylate (1.0 g, 4.97 mmol, 1.0 equiv.) and TEA (2.5 g, 24.9 mmol, 5.0 equiv.) in DCM (10 mL) was added MsCl (0.78 mL, 9.95 mmol, 2.0 equiv.) dropwise at 0° C. The reaction mixture was stirred at 0° C. for 3 h. TLC (PE/EA=1/1, ninhydrin) showed tert-butyl (2R,4R)-4-hydroxy-2-methyl-pyrrolidine-1-carboxylate was consumed completely and a new spot was formed. The mixture was diluted with water and extracted with DCM.

The combined organic layers were washed with brine, dried over Na₂SO₄ and the volatiles evaporated.

The residue was purified by flash column chromatography (10-50% EtOAc in PE) to yield tert-butyl rac-(2R,4R)-2-methyl-4-methylsulfonyloxy-pyrrolidine-1-carboxylate (1410 mg, 5.05 mmol, 96.5% yield) as a yellow oil. ¹H NMR (400 MHz, CDCl₃) δ=5.21-5.15 (m, 1H), 4.06-3.71 (m, 2H), 3.56 (br d, J=10.4 Hz, 1H), 3.03 (s, 3H), 2.45 (br d, J=1.6 Hz, 1H), 1.90-1.81 (m, 1H), 1.47 (s, 9H), 1.30-1.26 (m, 3H).

### Step 2: tert-butyl (2S,4R)-4-cyano-2-methyl-pyrrolidine-1-carboxylate

Error! Objects cannot be created from editing field codes.

To a mixture of tert-butyl (2S,4S)-2-methyl-4-methylsulfonyloxy-pyrrolidine-1-carboxylate (1.4 g, 5.0 mmol, 1.0 equiv.) in DMSO (15 mL) was added sodium cyanide (0.98 g, 20.0 mmol, 4.0 equiv.). The mixture was stirred at 80° C. for 16 h. The mixture was poured into aq. sat. NaHCO₃ and extracted with EtOAc. The combined organic layers were washed with brine, dried over Na₂SO₄ and the volatiles evaporated. The residue was purified by column chromatography (10-50% EtOAc in PE) to yield tert-butyl (2S,4R)-4-cyano-2-methyl-pyrrolidine-1-carboxylate (805 mg, 3.83 mmol, 72.6% yield) as a colorless oil. LC-MS: m/z=155.1 [M-56+H]⁺ ESI pos. ¹H NMR (400 MHz, CDCl₃) δ=5.21-5.15 (m, 1H), 4.06-3.71 (m, 2H), 3.56 (br d, J=10.4 Hz, 1H), 3.03 (s, 3H), 2.45 (br d, J=1.7 Hz, 1H), 1.90-1.81 (m, 1H), 1.47 (s, 9H), 1.30-1.26 (m, 3H). (NaCN work-up: Aqueous KOH (1M) was added to the combined aqueous phase to pH about 12. Then the mixture was poured into NaClO aqueous(5%, 1500 mL) and standing overnight and detected by analysis department recycled by special recycling bucket.)

### Step 3: (3R,5S)-5-methylpyrrolidine-3-carbonitrile; 2,2,2-trifluoroacetic acid

Error! Objects cannot be created from editing field codes.

To a solution of tert-butyl (2S,4R)-4-cyano-2-methyl-pyrrolidine-1-carboxylate (1.5 g, 7.13 mmol, 1.0 equiv.) in DCM (10 mL) was added TFA (10.0 mL, 123.25 mmol, 17.3 equiv.). The mixture was stirred at 25° C. for 2 h. The mixture was concentrated in vacuo to afford (3R,5S)-5-methylpyrrolidine-3-carbonitrile; 2,2,2-trifluoroacetic acid as light brown oil. The crude product was used into next step without further purification. ¹H NMR (400 MHz, DMSO-d₆) δ=3.63-3.43 (m, 4H), 2.60-2.52 (m, 1H), 1.86-1.76 (m, 1H), 1.32 (d, J=6.5 Hz, 3H).

### Step 4: (3R,5S)-1-(3-acetyl-6-chloro-2-pyridyl)-5-methyl-pyrrolidine-3-carbonitrile

Error! Objects cannot be created from editing field codes.

A solution of (3R,5S)-5-methylpyrrolidine-3-carbonitrile; 2,2,2-trifluoroacetic acid (used as crude from the previous step) and DIPEA (6.18 mL, 37.37 mmol, 5.59 equiv.) in DMSO (20 mL) was stirred at RT for 5 min. Then, 1-(6-chloro-2-fluoro-3-pyridyl)ethanone (CAS #1260663-13-5, 1.22 g, 7.05 mmol, 1.05 equiv., prepared in example 64, step 1) was added, and the reaction mixture stirred for 16 hours at RT. The reaction mixture was quenched with water and extracted with EtOAc. The combined organic layers were dried over Na₂SO₄ and concentrated under reduced pressure. The residue was purified by flash column chromatography (silica gel, 0-25% EtOAc in PE) to give (3R,5S)-1-(3-acetyl-6-chloro-2-pyridyl)-5-methyl-pyrrolidine-3-carbonitrile (1.2 g, 4.55 mmol, 60% yield) as light yellow oil. LC-MS: m/z=264.0 [M+H]⁺, ESI pos. ¹H NMR (400 MHz, CDCl₃) δ=7.84 (d, J=8.0 Hz, 1H), 6.74 (d, J=8.1 Hz, 1H), 4.48-4.39 (m, 1H), 3.75 (t, J=10.7 Hz, 1H), 3.06-2.97 (m, 1H), 2.95-2.84 (m, 1H), 2.67-2.59 (m, 1H), 2.55 (s, 3H), 2.03-1.96 (m, 1H), 1.36 (d, J=6.0 Hz, 3H).

### Step 5: mixture of (3R,5S)-1-[3-acetyl-6-[5-[(6-methylpyridazin-3-yl)amino]benzimidazol-1-yl]-2-pyridyl]-5-methyl-pyrrolidine-3-carbonitrile and (3R,5S)-1-[3-acetyl-6-[6-[(6-methylpyridazin-3-yl)amino]benzimidazol-1-yl]-2-pyridyl]-5-methyl-pyrrolidine-3-carbonitrile

Error! Objects cannot be created from editing field codes.

Prepared in analogy to example 64, step 2 using (3R,5S)-1-(3-acetyl-6-chloro-2-pyridyl)-5-methyl-pyrrolidine-3-carbonitrile (1.20 g, 4.6 mmol, 1.0 equiv.), N-(6-methylpyridazin-3-yl)-1H-benzimidazol-5-amine (1.32 g, 5.0 mmol, 1.1 equiv., prepared in example 64, intermediate 1) and K₂CO₃ (1.92 g, 13.9 mmol, 3.05 equiv.) Purification by preparative HPLC (Phenomenex Luna C18 250 mm×50 mm×10 μm, gradient 5-40% CH₃CN in H₂O (with 0.1% TFA) over 20 min, then 100% CH₃CN (2 min), flow rate 100 mL/min) to yield (3R,5S)-1-[3-acetyl-6-[5-[(6-methylpyridazin-3-yl)amino]benzimidazol-1-yl]-2-pyridyl]-5-methyl-pyrrolidine-3-carbonitrile (450 mg, 0.990 mmol, 21.9% yield) and (3R,5S)-1-[3-acetyl-6-[6-[(6-methylpyridazin-3-yl)amino]benzimidazol-1-yl]-2-pyridyl]-5-methyl-pyrrolidine-3-carbonitrile (800 mg, 1.760 mmol, 38.9% yield) as light-brown solids. Regioisomer 1: LC-MS: m/z=453.2 [M+H]⁺, ESI pos. ¹H NMR (400 MHz, CD₃OD) δ=9.05 (s, 1H), 8.38 (dd, J=3.0, 8.6 Hz, 2H), 8.22 (d, J=1.8 Hz, 1H), 7.91 (d, J=9.4 Hz, 1H), 7.73 (d, J=9.4 Hz, 1H), 7.55 (dd, J=2.1, 8.9 Hz, 1H), 7.27 (d, J=8.3 Hz, 1H), 4.68-4.58 (m, 1H), 3.73 (s, 1H), 3.24-3.15 (m, 2H), 2.76 (td, J=7.2, 12.5 Hz, 1H), 2.68-2.65 (m, 6H), 2.11-1.98 (m, 1H), 1.44 (d, J=6.1 Hz, 3H). Regioisomer 2: LC-MS: m/z=453.2 [M+H]⁺, ESI pos.

### Step 6: (3R,5S)-1-[3-(1-hydroxyethyl)-6-[5-[(6-methylpyridazin-3-yl)amino]benzimidazol-1-yl]-2-pyridyl]-5-methyl-pyrrolidine-3-carbonitrile; formic acid

Error! Objects cannot be created from editing field codes.

Prepared in analogy to example 53, step 4 using (3R,5S)-1-[3-acetyl-6-[5-[(6-methylpyridazin-3-yl)amino]benzimidazol-1-yl]-2-pyridyl]-5-methyl-pyrrolidine-3-carbonitrile (450 mg, 1.0 mmol, 1.0 equiv.) and NaBH₄ (113 mg, 3.0 mmol, 3.0 equiv.) at 20° C. to yield (3R,5S)-1-[3-(1-hydroxyethyl)-6-[5-[(6-methylpyridazin-3-yl)amino]benzimidazol-1-yl]-2-pyridyl]-5-methyl-pyrrolidine-3-carbonitrile; formic acid (91.4 mg, 0.180 mmol, 17.1% yield) as a yellow solid after preparative HPLC (Phenomenex C18 150 mm×25 mm×10 μm, gradient 10-4% CH₃CN in H₂O (with 0.225% formic acid) over 10 min, then 100% CH₃CN (2 min), flow rate 25 mL/min). LC-MS: m/z=455.2 [M+H]⁺, ESI pos. ¹H NMR (400 MHz, CD₃OD) δ=8.78 (br s, 1H), 8.36-8.25 (m, 1H), 8.24-8.04 (m, 3H), 7.63 (br d, J=8.9 Hz, 1H), 7.48-7.35 (m, 2H), 7.20-7.13 (m, 1H), 5.24-5.15 (m, 1H), 4.47-4.39 (m, 1H), 4.00-3.87 (m, 1H), 3.78-3.67 (m, 1H), 3.26-3.14 (m, 1H), 2.75-2.67 (m, 1H), 2.60-2.53 (m, 3H), 2.06-1.94 (m, 1H), 1.64 (br d, J=6.4 Hz, 2H), 1.52-1.42 (m, 1H), 1.35-1.25 (m, 3H).

### Example 245

### 1-[5-Chloro-3-(1-hydroxyethyl)-6-[5-[(6-methylpyridazin-3-yl)amino]benzimidazol-1-yl]-2-pyridyl]-5-methyl-pyrazole-3-carbonitrile; formic acid

Error! Objects cannot be created from editing field codes.

### Step 1: 1-[3-acetyl-5-chloro-6-[5-[(6-methylpyridazin-3-yl)amino]benzimidazol-1-yl]-2-pyridyl]-5-methyl-pyrazole-3-carbonitrile

Error! Objects cannot be created from editing field codes.

Prepared in analogy to example 239, step 2 using 1-[2,5-dichloro-6-[5-[(6-methylpyridazin-3-yl)amino]benzimidazol-1-yl]-3-pyridyl]ethanone (350.0 mg, 0.850 mmol, 1.0 equiv., prepared in example 239, step 1) and 5-methyl-1H-pyrazole-3-carbonitrile (90.71 mg, 0.850 mmol, 1.0 equiv.) to yield 1-[3-acetyl-5-chloro-6-[5-[(6-methylpyridazin-3-yl)amino]benzimidazol-1-yl]-2-pyridyl]-5-methyl-pyrazole-3-carbonitrile (40 mg, 0.080 mmol, 9.8% yield) as yellow solid. LC-MS: m/z=484.1 [M+H]⁺, ESI pos. ¹H NMR (400 MHz, CD₃OD) δ=8.70 (s, 1H), 8.64 (s, 1H), 8.27 (s, 1H), 7.63-7.61 (m, 2H), 7.38 (d, J=9.1 Hz, 1H), 7.15 (d, J=9.1 Hz, 1H), 6.87 (s, 1H), 2.58 (s, 3H), 2.55 (s, 3H), 2.35 (s, 3H).

### Step 2: 1-[5-chloro-3-(1-hydroxyethyl)-6-[5-[(6-methylpyridazin-3-yl)amino]benzimidazol-1-yl]-2-pyridyl]-5-methyl-pyrazole-3-carbonitrile; formic acid

Error! Objects cannot be created from editing field codes.

Prepared in analogy to example 53, step 4 using 1-[3-acetyl-5-chloro-6-[5-[(6-methylpyridazin-3-yl)amino]benzimidazol-1-yl]-2-pyridyl]-5-methyl-pyrazole-3-carbonitrile (20.0 mg, 0.040 mmol, 1.0 equiv.) and NaBH₄ (4.69 mg, 0.120 mmol, 3.0 equiv.) to yield 1-[5-chloro-3-(1-hydroxyethyl)-6-[5-[(6-methylpyridazin-3-yl)amino]benzimidazol-1-yl]-2-pyridyl]-5-methyl-pyrazole-3-carbonitrile; formic acid (11.3 mg, 0.020 mmol, 56.3% yield) as yellow solid. LC-MS: m/z=486.2 [M+H]⁺, ESI pos. ¹H NMR (400 MHz, CD₃OD) δ=8.64 (s, 1H), 8.62 (s, 1H), 8.30 (br s, 1H), 8.24 (d, J=1.8 Hz, 1H), 7.59 (dd, J=2.0, 8.9 Hz, 1H), 7.52-7.47 (m, 1H), 7.38 (d, J=9.3 Hz, 1H), 7.15 (d, J=9.1 Hz, 1H), 6.84 (d, J=0.8 Hz, 1H), 4.94 (s, 1H), 2.55 (s, 3H), 2.40 (d, J=0.6 Hz, 3H), 1.42 (d, J=6.4 Hz, 3H).

### Example 246

### 5-[3-(1-Hydroxyethyl)-6-[5-[(6-methylpyridazin-3-yl)amino]benzimidazol-1-yl]-2-pyridyl]-2-methyl-pyrazole-3-carbonitrile; formic acid

Error! Objects cannot be created from editing field codes.

### Step 1: 5-bromo-2-methyl-pyrazole-3-carbonitrile

Error! Objects cannot be created from editing field codes.

To a solution of 5-bromo-2-methyl-pyrazole-3-carboxamide (500.0 mg, 2.45 mmol, 1.0 equiv.) and TEA (1.02 mL, 7.35 mmol, 3 equiv.) in DCM (10 mL), was added TFAA (0.41 mL, 2.94 mmol, 1.2 equiv.) slowly at 0° C., and the reaction was stirred at 30° C. for 12 h. The mixture was poured into water. The aqueous phase was extracted with EtOAc). The combined organic layers were washed with, dried over anhydrous Na₂SO₄ and concentrated under reduced pressure. The residue was purified by flash column chromatography (silica gel, 5% EtOAc in DCM) to afford 5-bromo-2-methyl-pyrazole-3-carbonitrile (400 mg, 2.15 mmol, 86.0% yield) as a white solid. ¹H NMR (400 MHz, CDCl₃) δ=6.77-6.83 (m, 1H) 4.04-4.09 (m, 3H).

### Step 2: 2-methyl-5-(4,4,5,5-tetramethyl-1,3,2-dioxaborolan-2-yl)pyrazole-3-carbonitrile

Error! Objects cannot be created from editing field codes.

Prepared in analogy to example 143, step 2 using 5-bromo-2-methyl-pyrazole-3-carbonitrile (100.0 mg, 0.540 mmol, 1.0 equiv.) and 5-bromo-2-methyl-pyrazole-3-carbonitrile (100.0 mg, 0.540 mmol, 1.0 equiv.) to yield 2-methyl-5-(4,4,5,5-tetramethyl-1,3,2-dioxaborolan-2-yl)pyrazole-3-carbonitrile (100 mg, 0.430 mmol, 76.62% yield) a brown solid after purification by preparative TLC (25% EtOAc in PE). ¹H NMR (400 MHz, CDCl₃) δ=7.15-7.17 (m, 1H) 2.05 (s, 3H) 1.35-1.37 (m, 12H).

### Step 3: 5-[3-acetyl-6-[5-[(6-methylpyridazin-3-yl)amino]benzimidazol-1-yl]-2-pyridyl]-2-methyl-pyrazole-3-carbonitrile

Error! Objects cannot be created from editing field codes.

Prepared in analogy to example 143, step 2 using 1-[2-chloro-6-[5-[(6-methylpyridazin-3-yl)amino]benzimidazol-1-yl]-3-pyridyl]ethanone (50.0 mg, 0.130 mmol, 1.0 equiv., prepared in example 65, step 1)and 2-methyl-5-(4,4,5,5-tetramethyl-1,3,2-dioxaborolan-2-yl)pyrazole-3-carbonitrile (46.14 mg, 0.200 mmol, 1.5 equiv.) to give 5-[3-acetyl-6-[5-[(6-methylpyridazin-3-yl)amino]benzimidazol-1-yl]-2-pyridyl]-2-methyl-pyrazole-3-carbonitrile (15 mg, 0.030 mmol, 24.3% yield) as a white solid. LC-MS: m/z=450. 2 [M+H]⁺, ESI pos. ¹H NMR (400 MHz, DMSO-d₆) δ ppm 9.22-9.26 (m, 1H) 9.07-9.12 (m, 1H) 8.34-8.42 (m, 2H) 8.11-8.15 (m, 1H) 8.05-8.08 (m, 1H) 7.60-7.64 (m, 1H) 7.32-7.37 (m, 1H)7.08-7.14 (m, 1H)4.10-4.13 (m, 3H)2.41-2.43(m, 3H).

### Step 4:5-[3-(1-hydroxyethyl)-6-[5-[(6-methylpyridazin-3-yl)amino]benzimidazol-1-yl]-2-pyridyl]-2-methyl-pyrazole-3-carbonitrile

Error! Objects cannot be created from editing field codes.

Prepared in analogy to example 53, step 4 using 5-[3-acetyl-6-[5-[(6-methylpyridazin-3-yl)amino]benzimidazol-1-yl]-2-pyridyl]-2-methyl-pyrazole-3-carbonitrile (15.0 mg, 0.030 mmol, 1.0 equiv.) to give 5-[3-(1-hydroxyethyl)-6-[5-[(6-methylpyridazin-3-yl)amino]benzimidazol-1-yl]-2-pyridyl]-2-methyl-pyrazole-3-carbonitrile; formic acid (4 mg, 0.010 mmol, 26.4% yield) as a white solid. LC-MS: m/z=452.1 [M+H]⁺, ESI pos. ¹H NMR (400 MHz, CD₃OD) δ ppm 8.82-8.88 (m, 1H) 8.33-8.38 (m, 1H) 8.14-8.23 (m, 2H) 7.79-7.83 (m, 1H) 7.60-7.65 (m, 1H) 7.52-7.56 (m, 1H) 7.35-7.41 (m, 1H) 7.13-7.19 (m, 1H) 5.78-5.86 (m, 1H) 4.14-4.20 (m, 3H) 2.53-2.59 (m, 3H) 1.51-1.57 (m, 3H).

### Example 247

### 1-[3-[(1R)-1-hydroxyethyl]-6-[6-[(6-methylpyridazin-3-yl)amino]benzimidazol-1-yl]-2-pyridyl]-5-methyl-pyrazole-3-carbonitrile

Error! Objects cannot be created from editing field codes.

1-[3-(1-hydroxyethyl)-6-[6-[(6-methylpyridazin-3-yl)amino]benzimidazol-1-yl]-2-pyridyl]-5-methyl-pyrazole-3-carbonitrile (obtained as in step 3 of example 64) (400 mg, 0.89 mmol, 1 equiv.) was purified by chiral SFC (Daicel Chiralpak AY-H (250 mm×30 mm×10 μm). Flow rate: 70 mL/min. 60% (0.1% NH₄OH in isopropanol)). The title compound (124.2 mg, 0.275 mmol, 30.9% yield) was obtained as a white solid. LC-MS: m/z=452.2 [M+H]⁺, ESI pos. ¹H NMR (400 MHz, CD₃OD): δ=8.89 (s, 1H), 8.50 (d, J=8.4 Hz, 1H), 8.22 (d, J=2.0 Hz, 1H), 8.16-8.08 (m, 2H), 7.59 (dd, J=2.1, 8.9 Hz, 1H), 7.35 (d, J=9.3 Hz, 1H), 7.12 (d, J=9.1 Hz, 1H), 6.87 (s, 1H), 4.79-4.74 (m, 1H), 2.53 (s, 3H), 2.41 (s, 3H), 1.41 (d, J=6.5 Hz, 3H). Assignment of the absolute stereochemistry was done in analogy to examples 109 and 110.

### Example 248

### 1-[2-[3,5-Bis(difluoromethyl)pyrazol-1-yl]-6-[5-[(6-methylpyridazin-3-yl)amino]benzimidazol-1-yl]-3-pyridyl]ethanol

Error! Objects cannot be created from editing field codes.

### Step 1: 1-[2-fluoro-6-[5-[(6-methylpyridazin-3-yl)amino]benzimidazol-1-yl]-3-pyridyl]ethanone

Error! Objects cannot be created from editing field codes.

1H-benzimidazol-5-yl-(6-methylpyridazin-3-yl)amine (4.05 g, 15.27 mmol, 1.2 equiv.) and K₂CO₃ (3.52 g, 25.46 mmol, 2.0 equiv.) were suspended in DMF (65 mL) and cooled to −20° C. 1-(2,6-difluoro-3-pyridyl)ethanone (2.0 g, 1.45 mL, 12.73 mmol, 1.0 equiv.) was added dropwise and the reaction allowed to stir for 3 h. The reaction was allowed to warm to rt, water and DCM were added which resulted in the formation of a suspension. The mixture was filtered. The filter cake was dissolved in DMF and reprecipitated on ice. After filtration, the filter cake is adsorbed on isolute, and purified by column chromatography (silica gel, 5-50% MeOH/NH₄OH (9:1) in MeOH to yield 1-[2-fluoro-6-[5-[(6-methylpyridazin-3-yl)amino]benzimidazol-1-yl]-3-pyridyl]ethanone (1.71 g, 34.4%) as yellow solid. LC-MS: m/z=363.2 [M+H]⁺, ESI pos.

### Step 2: 1-[2-[3,5-bis(difluoromethyl)pyrazol-1-yl]-6-[5-[(6-methylpyridazin-3-yl)amino]benzimidazol-1-yl]-3-pyridyl]ethanone

Error! Objects cannot be created from editing field codes.

1-[2-fluoro-6-[5-[(6-methylpyridazin-3-yl)amino]benzimidazol-1-yl]-3-pyridyl]ethanone (30 mg, 0.083 mmol, 1.0 equiv.) was dissolved in DMF (1 mL) and 3,5-bis(difluoromethyl)-1H-pyrazole (15.31 mg, 0.091 mmol, 1.1 equiv.) and K₂CO₃ (17.16 mg, 0.124 mmol, 1.5 equiv.) were added at rt. The mixture was stirred for 5 h at rt. LC-MS showed reaction was finished. The reaction mixture was diluted with water and extracted two times with EtOAc. The organic layers were washed with brine, dried over MgSO₄ and concentrated to dryness to obtain the crude intermediate 1-[2-[3,5-bis(difluoromethyl)pyrazol-1-yl]-6-[5-[(6-methylpyridazin-3-yl)amino]benzimidazol-1-yl]-3-pyridyl]ethanone as light yellow solid which was used in the next step without further purification.

### Step 3:1-[2-[3,5-bis(difluoromethyl)pyrazol-1-yl]-6-[5-[(6-methylpyridazin-3-yl)amino]benzimidazol-1-yl]-3-pyridyl]ethanol

Error! Objects cannot be created from editing field codes.

Prepared in analogy to example 53, step 4 using 1-[2-[3,5-bis(difluoromethyl)pyrazol-1-yl]-6-[5-[(6-methylpyridazin-3-yl)amino]benzimidazol-1-yl]-3-pyridyl]ethanone (used as crude from the previous step) in ⁱPrOH/THF (1:1) to yield 1-[2-[3,5-bis(difluoromethyl)pyrazol-1-yl]-6-[5-[(6-methylpyridazin-3-yl)amino]benzimidazol-1-yl]-3-pyridyl]ethanol (24.5 mg, 57.3%) as light yellow solid. LC-MS: m/z=513.27 [M+H]⁺, ESI pos.

### Example 249

### 1-[3-(1-Hydroxyethyl)-6-[5-[[6-(trifluoromethyl)pyridazin-3-yl]amino]benzimidazol-1-yl]-2-pyridyl]-5-methyl-pyrazole-3-carbonitrile

Error! Objects cannot be created from editing field codes.

### Step 1: 1-[3-acetyl-6-[5-[[6-(trifluoromethyl)pyridazin-3-yl]amino]benzimidazol-1-yl]-2-pyridyl]-5-methyl-pyrazole-3-carbonitrile

Error! Objects cannot be created from editing field codes.

Prepared in analogy to example 104, step 2 using 1-[3-acetyl-6-(5-bromobenzimidazol-1-yl)-2-pyridyl]-5-methyl-pyrazole-3-carbonitrile (30 mg, 0.071 mmol, 1.0 equiv., prepared in example 65, step 1) and [6-(trifluoromethyl)pyridazin-3-yl]amine (23.23 mg, 0.142 mmol, 2.0 equiv.) to yield the title compound (23.4 mg, 58.7% yield) as a light yellow solid. LC-MS: m/z=504.3 [M+H]⁺, ESI pos.

### Step 2: 1-[3-(-hydroxyethyl)-6-[5-[[6-(trifluoromethyl)pyridazin-3-yl]amino]benzimidazol-1-yl]-2-pyridyl]-5-methyl-pyrazole-3-carbonitrile

Error! Objects cannot be created from editing field codes.

Prepared in analogy to example 53, step 4 using 1-[3-acetyl-6-[5-[[6-(trifluoromethyl)pyridazin-3-yl]amino]benzimidazol-1-yl]-2-pyridyl]-5-methyl-pyrazole-3-carbonitrile (23.4 mg, 0.046 mmol, 1 equiv.) in MeOH/THF (1:1) to yield the title compound (22.2 mg, 91.7% yield) as light yellow solid. LC-MS: m/z=506.3 [M+H]⁺, ESI pos.

### Example 250

### 1-[2-[2-Ethyl-5-(trifluoromethyl)pyrazol-3-yl]-6-[5-[(6-methylpyridazin-3-yl)amino]benzimidazol-1-yl]-3-pyridyl]ethanol

Error! Objects cannot be created from editing field codes.

### Step 1: 5-bromo-1-ethyl-3-(trifluoromethyl)pyrazole

Error! Objects cannot be created from editing field codes.

To a solution of 5-bromo-3-(trifluoromethyl)-1H-pyrazole (500.0 mg, 2.33 mmol, 1.0 equiv.) in 1,4-dioxane (10 mL) was added iodoethane (0.28 mL, 3.49 mmol, 1.5 equiv.) and K₂CO₃ (0.96 g, 6.98 mmol, 3.0 equiv.). The reaction mixture was stirred at 100° C. for 12 h. The mixture was cooled to RT and filtered. The filtrate was used without further purification in the next step. LC-MS: m/z=245.0 [M+H]⁺, ESI pos.

### Step 2: 1-ethyl-5-(4,4,5,5-tetramethyl-1,3,2-dioxaborolan-2-yl)-3-(trifluoromethyl)pyrazole

Error! Objects cannot be created from editing field codes.

Prepared in analogy to example 109, step 3 using 5-bromo-1-ethyl-3-(trifluoromethyl)pyrazole used as crude from the previous step) and bis(pinacolato)diboron (783 mg, 3.100 mmol, 1.5 equiv.). After the reaction was completed, the mixture was cooled to RT and filtered. The filtrate was evaporated to dryness to yield 1-ethyl-5-(4,4,5,5-tetramethyl-1,3,2-dioxaborolan-2-yl)-3-(trifluoromethyl)pyrazole (153.2 mg, 0.530 mmol, 22.7% yield over 2 steps) as brown liquid which was used in next step without further purification. LC-MS: m/z=327.0 [M+H]⁺, ESI pos.

### Step 3: 1-[2-[2-ethyl-5-(trifluoromethyl)pyrazol-3-yl]-6-[5-[(6-methylpyridazin-3-yl)amino]benzimidazol-1-yl]-3-pyridyl]ethanone

Error! Objects cannot be created from editing field codes.

Prepared in analogy to example 143, step 2 using 1-ethyl-5-(4,4,5,5-tetramethyl-1,3,2-dioxaborolan-2-yl)-3-(trifluoromethyl)pyrazole (153.2 mg, 0.530 mmol, 2.0 equiv.) and 1-[2-chloro-6-[5-[(6-methylpyridazin-3-yl)amino]benzimidazol-1-yl]-3-pyridyl]ethanone (100.0 mg, 0.260 mmol, 1.0 equiv., prepared in example 76, step 2) to yield 1-[2-[2-ethyl-5-(trifluoromethyl)pyrazol-3-yl]-6-[5-[(6-methylpyridazin-3-yl)amino]benzimidazol-1-yl]-3-pyridyl]ethanone (10 mg, 0.020 mmol, 7.1% yield). LC-MS: m/z=509.0 [M+H]⁺, ESI pos.

### Step 4: 1-[2-[2-ethyl-5-(trifluoromethyl)pyrazol-3-yl]-6-[5-[(6-methylpyridazin-3-yl)amino]benzimidazol-1-yl]-3-pyridyl]ethanol

Error! Objects cannot be created from editing field codes.

Prepared in analogy to example 53, step 4 using 1-[2-[2-ethyl-5-(trifluoromethyl)pyrazol-3-yl]-6-[5-[(6-methylpyridazin-3-yl)amino]benzimidazol-1-yl]-3-pyridyl]ethanone (10.0 mg, 0.020 mmol, 1.0 equiv.) and NaBH₄ (1.12 mg, 0.030 mmol, 1.5 equiv.) at −70° C. to give 1-[2-[2-ethyl-5-(trifluoromethyl)pyrazol-3-yl]-6-[5-[(6-methylpyridazin-3-yl)amino]benzimidazol-1-yl]-3-pyridyl]ethanol (3.73 mg, 0.010 mmol, 37.2% yield) as white solid LC-MS: m/z=509.0 [M+H]⁺, ESI pos. ¹H NMR (400 MHz, CD₃OD) δ=8.89 (s, 1H), 8.40 (d, J=8.6 Hz, 1H), 8.21 (d, J=1.5 Hz, 1H), 8.14 (d, J=8.9 Hz, 1H), 8.04 (d, J=8.7 Hz, 1H), 7.59-7.53 (m, 1H), 7.43 (d, J=9.2 Hz, 1H), 7.21 (d, J=9.2 Hz, 1H), 6.88 (s, 1H), 4.96-4.93 (m, 1H), 4.26 (dq, J=2.0, 7.1 Hz, 2H), 2.55 (s, 3H), 1.45 (d, J=6.5 Hz, 3H), 1.39 (t, J=7.2 Hz, 3H).

### Example 251

### (1R)-1-[6-[5-[(6-Methylpyridazin-3-yl)amino]benzimidazol-1-yl]-2-[3-methyl-1-(2,2,2-trifluoroethyl)pyrazol-4-yl]-3-pyridyl]ethanol

Error! Objects cannot be created from editing field codes.

The enantiomers of 1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanol (450.0 mg, 0.880 mmol, 1.0 equiv., from Example 240, step 2) were separated by chiral SFC (Daicel ChiralPak IG 250 mm×30 mm, 10 μm, ⁱPrOH (with 0.1% ammonium hydroxide) (1R)-1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanol (211.9 mg, 0.420 mmol, 47.1% yield, 99.4% ee) as light yellow solid. LC-MS: m/z=509.2 [M+H]⁺, ESI pos. ¹H NMR (400 MHz, CD₃OD) δ=8.83 (s, 1H), 8.28 (d, J=8.4 Hz, 1H), 8.19 (d, J=1.6 Hz, 1H), 8.15 (d, J=8.8 Hz, 1H), 8.01 (s, 1H), 7.83 (d, J=8.4 Hz, 1H), 7.62-7.52 (m, 1H), 7.35 (d, J=9.2 Hz, 1H), 7.12 (d, J=9.2 Hz, 1H), 5.06-4.95 (m, 3H), 2.52 (s, 3H), 2.35 (s, 3H), 1.47 (d, J=6.4 Hz, 3H). Assignment of the absolute stereochemistry was done in analogy to examples 109 and 110.

### Example 252

### (1S)-1-[6-[5-[(6-Methylpyridazin-3-yl)amino]benzimidazol-1-yl]-2-[3-methyl-1-(2,2,2-trifluoroethyl)pyrazol-4-yl]-3-pyridyl]ethanol

Error! Objects cannot be created from editing field codes.

The enantiomers of 1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanol (450.0 mg, 0.880 mmol, 1.0 equiv., from Example 240, step 2) were separated by chiral SFC (Daicel ChiralPak IG 250 mm×30 mm, 10 μm, ⁱPrOH (with 0.1% ammonium hydroxide)) (1S)-1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanol (206.3 mg, 0.410 mmol, 45.8% yield, 100% ee) as light yellow solid. LC-MS: m/z=509.2 [M+H]⁺, ESI pos. ¹H NMR (400 MHz, CD₃OD) δ=8.84 (s, 1H), 8.30 (s, 1H), 8.20 (d, J=2.0 Hz, 1H), 8.16 (d, J=8.8 Hz, 1H), 8.02 (s, 1H), 7.84 (d, J=8.4 Hz, 1H), 7.65-7.54 (m, 1H), 7.36 (d, J=9.2 Hz, 1H), 7.13 (d, J=9.2 Hz, 1H), 5.09-4.98 (m, 3H), 2.53 (s, 3H), 2.35 (s, 3H), 1.47 (d, J=6.4 Hz, 3H). Assignment of the absolute stereochemistry was done in analogy to examples 109 and 110.

### Example 253

### 1-[3-(1-Hydroxyethyl)-6-[5-[(6-methylpyridazin-3-yl)amino]benzimidazol-1-yl]-2-pyridyl]triazole-4-carbonitrile

Error! Objects cannot be created from editing field codes.

### Step 1: 2-[3-acetyl-6-[5-[(6-methylpyridazin-3-yl)amino]benzimidazol-1-yl]-2-pyridyl]triazole-4-carbonitrile, and 1-[3-acetyl-6-[5-[(6-methylpyridazin-3-yl)amino]benzimidazol-1-yl]-2-pyridyl]triazole-4-carbonitrile, and 3-[3-acetyl-6-[5-[(6-methylpyridazin-3-yl)amino]benzimidazol-]-yl]-2-pyridyl]triazole-4-carbonitrile

Error! Objects cannot be created from editing field codes.

A solution of 1-[2-chloro-6-[5-[(6-methylpyridazin-3-yl)amino]benzimidazol-1-yl]-3-pyridyl]ethanone (300.0 mg, 0.790 mmol, 1.0 equiv., prepared in example 76, step 2), 2H-triazole-4-carbonitrile (90.0 mg, 0.960 mmol, 1.21 equiv.) and DIPEA (0.42 mL, 2.55 mmol, 3.22 equiv.) in DMSO (6 mL) was stirred at 100° C. for 12 h. The reaction was poured into water and a dark brown solid was precipitated out. After filtration, the filter cake was dried under reduced pressure. The filtrate was extracted with and the combined organic layers were washed with brine. The organic layer and filter cake were combined and concentrated under vacuum. The residue was purified by preparative NPLC (Welch Ultimate XB-CN 250 mm×70 mm×10 μm, gradient 60-100% EtOH in hexane over 15 min, then 100% EtOH (2 min), flow rate 140 mL/min) followed by preparative HPLC (Phenomenex Synergi Poar-RP 100 mm×25 mm×4 μm, gradient 24-44% CH₃CN in H₂O (with 0.225% formic acid) over 7 min, then 100% CH₃CN (2 min), flow rate 25 mL/min, 1 injection) to afford 2-[3-acetyl-6-[5-[(6-methylpyridazin-3-yl)amino]benzimidazol-1-yl]-2-pyridyl]triazole-4-carbonitrile (30 mg, 0.070 mmol, 8.68% yield) as white solid, 1-[3-acetyl-6-[5-[(6-methylpyridazin-3-yl)amino]benzimidazol-1-yl]-2-pyridyl]triazole-4-carbonitrile (20 mg, 0.050 mmol, 5.8% yield) and 3-[3-acetyl-6-[5-[(6-methylpyridazin-3-yl)amino]benzimidazol-1-yl]-2-pyridyl]triazole-4-carbonitrile (15 mg, 0.030 mmol, 4.2% yield) as white solids. Regioisomer 1: LC-MS: m/z=437.1 [M+H]⁺, ESI pos. ¹H NMR (400 MHz, DMSO-d₆) δ=9.93 (s, 1H), 9.28 (s, 1H), 9.19 (s, 1H), 8.63 (d, J=8.5 Hz, 1H), 8.46 (d, J=1.9 Hz, 1H), 8.37 (d, J=8.6 Hz, 1H), 8.28 (d, J=8.9 Hz, 1H), 7.60 (dd, J=2.1, 8.9 Hz, 1H), 7.36 (d, J=9.1 Hz, 1H), 7.11 (d, J=9.1 Hz, 1H), 2.50-2.49 (m, 3H), 2.48 (s, 3H). Regioisomer 2: LC-MS: m/z=437.1 [M+H]⁺, ESI pos. ¹H NMR (400 MHz, DMSO-d₆) δ=10.20-10.08 (m, 1H), 9.15 (s, 1H), 8.51 (d, J=8.3 Hz, 1H), 8.43 (d, J=8.9 Hz, 1H), 8.33 (s, 1H), 8.16 (d, J=8.4 Hz, 1H), 7.86-7.77 (m, 1H), 7.60 (br d, J=8.4 Hz, 1H), 7.55 (dd, J=2.1, 8.9 Hz, 1H), 7.01-6.87 (m, 1H), 2.59 (s, 3H), 1.92 (s, 3H). Regioisomer 3: LC-MS: m/z=437.1 [M+H]⁺, ESI pos. ¹H NMR (400 MHz, DMSO-d₆) δ=10.17 (br s, 1H), 9.25 (s, 1H), 9.02 (s, 1H), 8.61 (d, J=8.4 Hz, 1H), 8.52 (d, J=8.9 Hz, 1H), 8.40 (d, J=8.6 Hz, 1H), 8.25 (d, J=1.3 Hz, 1H), 7.83 (br d, J=8.9 Hz, 1H), 7.64-7.59 (m, 1H), 7.56 (dd, J=2.0, 9.0 Hz, 1H), 2.59 (s, 3H), 2.49-2.49 (m, 3H).

### Step 2: 1-[3-(1-hydroxyethyl)-6-[5-[(6-methylpyridazin-3-yl)amino]benzimidazol-1-yl]-2-pyridyl]triazole-4-carbonitrile

Error! Objects cannot be created from editing field codes.

Prepared in analogy to example 53, step 4 suing 1-[3-acetyl-6-[5-[(6-methylpyridazin-3-yl)amino]benzimidazol-1-yl]-2-pyridyl]triazole-4-carbonitrile (30.0 mg, 0.070 mmol, 1.0 equiv.) and NaBH₄ (8.0 mg, 0.210 mmol, 3.1 equiv.) in DCM/MeOH (1:1) to afford 1-[3-(1-hydroxyethyl)-6-[5-[(6-methylpyridazin-3-yl)amino]benzimidazol-1-yl]-2-pyridyl]triazole-4-carbonitrile (11.3 mg, 0.030 mmol, 37.5% yield) as yellow solid. Purification by HPLC (Waters Xbridge 150×25 mm×5 um), water (10 mM NH₄HCO₃)-ACN, 24%-54%), flow rate 25 mL/min). LCMS:439.1[M+H]⁺ ESI pos. ¹H NMR (400 MHz, DMSO-d₆) δ=9.84 (s, 1H), 9.23 (s, 1H), 9.07 (s, 1H), 8.53 (d, J=8.4 Hz, 1H), 8.42 (d, J=2.0 Hz, 1H), 8.29 (d, J=8.6 Hz, 1H), 8.15 (d, J=8.9 Hz, 1H), 7.54 (dd, J=2.1, 8.9 Hz, 1H), 7.34 (d, J=9.0 Hz, 1H), 7.09 (d, J=9.0 Hz, 1H), 5.60 (br s, 1H), 5.00 (q, J=6.4 Hz, 1H), 2.48 (br s, 3H), 1.40 (d, J=6.4 Hz, 3H).

### Example 254

### (3S,5R)-1-[3-(1-hydroxyethyl)-6-[5-[(6-methylpyridazin-3-yl)amino]benzimidazol-1-yl]-2-pyridyl]-5-methyl-pyrrolidine-3-carbonitrile

Error! Objects cannot be created from editing field codes.

### Step 1: tert-butyl (2R,4R)-2-methyl-4-methylsulfonyloxy-pyrrolidine-1-carboxylate

Error! Objects cannot be created from editing field codes.

Prepared in analogy to example 242, step 1 using tert-butyl (2R,4R)-4-hydroxy-2-methyl-pyrrolidine-1-carboxylate (1.0 g, 4.97 mmol, 1.0 equiv.), TEA (2.5 g, 24.9 mmol, 5.0 equiv.) and MsCl (0.78 mL, 9.95 mmol, 2.0 equiv.) in DCM (10 mL) to give tert-butyl (2R,4R)-2-methyl-4-methylsulfonyloxy-pyrrolidine-1-carboxylate (1.41 g, 5.05 mmol, 96.5% yield) as a yellow oil. ¹H NMR (400 MHz, CDCl₃) δ=5.21-5.15 (m, 1H), 4.06-3.71 (m, 2H), 3.56 (br d, J=10.4 Hz, 1H), 3.03 (s, 3H), 2.45 (br d, J=1.6 Hz, 1H), 1.90-1.81 (m, 1H), 1.47 (s, 9H), 1.30-1.26 (m, 3H).

### Step 2: tert-butyl (2R,4S)-4-cyano-2-methyl-pyrrolidine-1-carboxylate

Error! Objects cannot be created from editing field codes.

Prepared in analogy to example 242, step 2 using tert-butyl (2R,4R)-2-methyl-4-methylsulfonyloxy-pyrrolidine-1-carboxylate (1.4 g, 5.1 mmol, 1.0 equiv.), sodium cyanide (0.99 g, 20.2 mmol, 4.0 equiv.) and DMSO (15 mL) to yield tert-butyl (2R,4S)-4-cyano-2-methyl-pyrrolidine-1-carboxylate (660.0 mg, 3.14 mmol, 62.4% yield) as a colorless oil, which was used without purification in the next step. LC-MS: m/z=155.1 [M-56+H]⁺ ESI pos. ¹H NMR (400 MHz, CDCl₃) δ=5.21-5.15 (m, 1H), 4.06-3.71 (m, 2H), 3.56 (br d, J=10.4 Hz, 1H), 3.03 (s, 3H), 2.45 (br d, J=1.7 Hz, 1H), 1.90-1.81 (m, 1H), 1.47 (s, 9H), 1.30-1.26 (m, 3H).

### Step 3: (3S,5R)-5-methylpyrrolidine-3-carbonitrile; 2,2,2-trifluoroacetic acid

Error! Objects cannot be created from editing field codes.

Prepared in analogy to example 242, step 3 using tert-butyl (2R,4S)-4-cyano-2-methyl-pyrrolidine-1-carboxylate (660.0 mg, 3.14 mmol, 1.0 equiv.) and trifluoroacetic acid (4.1 mL, 53.4 mmol, 17 equiv.) and DCM (5 mL) to yield (3S,5R)-5-methylpyrrolidine-3-carbonitrile (310 mg, 2.81 mmol, 89.5% yield) as a red oil which was used without purification in the next step.

### Step 4: (3S,5R)-1-(3-acetyl-6-methyl-2-pyridyl)-5-methyl-pyrrolidine-3-carbonitrile

Error! Objects cannot be created from editing field codes.

Prepared in analogy to example 105, step 1 using 1-(6-chloro-2-fluoro-3-pyridyl)ethanone (CAS #1260663-13-5, 745.3 mg, 4.3 mmol, 1.1 equiv.) and (3S,5R)-5-methylpyrrolidine-3-carbonitrile (430.0 mg, 3.9 mmol, 1.0 equiv.) to give (3S,5R)-1-(3-acetyl-6-methyl-2-pyridyl)-5-methyl-pyrrolidine-3-carbonitrile (330 mg, 1.36 mmol, 34.75% yield) as a yellow oil. LC-MS: m/z=264.0 [M+H]⁺, ESI pos. ¹H NMR (400 MHz, DMSO-d₆) δ=8.10 (d, J=8.0 Hz, 1H), 6.84 (d, J=8.0 Hz, 1H), 4.29-4.20 (m, 1H), 3.44-3.38 (m, 1H), 3.26-3.18 (m, 1H), 3.02 (dd, J=7.2, 10.0 Hz, 1H), 2.61-2.57 (m, 1H), 2.55 (s, 3H), 1.95-1.84 (m, 1H), 1.26 (d, J=6.0 Hz, 3H)

### Step 5: (3S,5R)-1-(3-acetyl-6-chloro-2-pyridyl)-5-methyl-pyrrolidine-3-carbonitrile

Error! Objects cannot be created from editing field codes.

Prepared in analogy to example 64, step 2 using (3S,5R)-1-(3-acetyl-6-chloro-2-pyridyl)-5-methyl-pyrrolidine-3-carbonitrile (670.0 mg, 2.54 mmol, 1.0 equiv.) and N-(6-methylpyridazin-3-yl)-1H-benzimidazol-5-amine (572.26 mg, 2.54 mmol, 1.0 equiv., prepared in example 64, intermediate 1) to yield (3S,5R)-1-(3-acetyl-6-chloro-2-pyridyl)-5-methyl-pyrrolidine-3-carbonitrile (400 mg, 1.52 mmol, 59.6% yield) as a yellow solid after separation of the isomers by preparative HPLC (Phenomenex Luna 75 mm×30 mm×3 μm, gradient 22-42% CH₃CN in H₂O (with 0.1% TFA) over 7 min, then 100% CH₃CN (2 min), flow rate 25 mL/min). NO LC-MS H NMR (400 MHz, DMSO-d₆) δ=1.17 (d, J=5.99 Hz, 3H) 1.87-1.97 (m, 1H) 2.61 (d, J=7.95 Hz, 7H) 3.31 (br d, J=10.15 Hz, 1H) 3.36-3.41 (m, 1H) 3.56 (s, 1H) 4.48-4.57 (m, 1H) 7.28-7.37 (m, 2H) 7.64 (br d, J=9.29 Hz, 1H) 7.75-7.83 (m, 2H) 8.35-8.38 (m, 1H) 8.93-9.05 (m, 2H) 10.30 (br s, 1H).

### Step 6: (3S,5R)-1-[3-(1-hydroxyethyl)-6-[5-[(6-methylpyridazin-3-yl)amino]benzimidazol-1-yl]-2-pyridyl]-5-methyl-pyrrolidine-3-carbonitrile

Error! Objects cannot be created from editing field codes.

Prepared in analogy to example 53, step 4 using (3S,5R)-1-[3-acetyl-6-[5-[(6-methylpyridazin-3-yl)amino]benzimidazol-1-yl]-2-pyridyl]-5-methyl-pyrrolidine-3-carbonitrile (70.0 mg, 0.150 mmol, 1.0 equiv.) and NaBH₄ (50.0 mg, 1.32 mmol, 8.5 equiv.) at RT to yield formic acid; (3S,5R)-1-[3-(1-hydroxyethyl)-6-[5-[(6-methylpyridazin-3-yl)amino]benzimidazol-1-yl]-2-pyridyl]-5-methyl-pyrrolidine-3-carbonitrile (15.3 mg, 0.030 mmol, 20.7% yield) as a yellow solid. LC-MS: m/z=455.2 [M+H]⁺, ESI pos. ¹H NMR (400 MHz, CD₃OD) δ=8.78 (d, J=2.0 Hz, 1H), 8.35-8.26 (m, 1H), 8.21-8.05 (m, 3H), 7.68-7.60 (m, 1H), 7.47-7.35 (m, 2H), 7.16 (d, J=9.2 Hz, 1H), 5.25-5.16 (m, 1H), 4.54-4.41 (m, 1H), 4.04-3.86 (m, 1H), 3.82-3.66 (m, 1H), 3.29 (br s, 1H), 2.71 (dd, J=6.8, 12.5 Hz, 1H), 2.55 (s, 3H), 2.06-1.92 (m, 1H), 1.64 (d, J=6.4 Hz, 2H), 1.46 (d, J=6.4 Hz, 2H), 1.30 (t, J=5.6 Hz, 3H).

### Example 255

### 5-[3-(1-hydroxyethyl)-6-[5-[(6-methylpyridazin-3-yl)amino]benzimidazol-1-yl]-2-pyridyl]-1-methyl-pyrazole-3-carbonitrile

Error! Objects cannot be created from editing field codes.

### Step 1: 5-[3-acetyl-6-[5-[(6-methylpyridazin-3-yl)amino]benzimidazol-1-yl]-2-pyridyl]-1-methyl-pyrazole-3-carbonitrile

Error! Objects cannot be created from editing field codes.

Prepared in analogy to example 143, step 2 using 1-[2-chloro-6-[5-[(6-methylpyridazin-3-yl)amino]benzimidazol-1-yl]-3-pyridyl]ethanone (50.0 mg, 0.130 mmol, 1.0 equiv., prepared in example 76, step 2) and 1-methyl-5-(4,4,5,5-tetramethyl-1,3,2-dioxaborolan-2-yl)pyrazole-3-carbonitrile (46.14 mg, 0.200 mmol, 1.5 equiv.) to give 5-[3-acetyl-6-[5-[(6-methylpyridazin-3-yl)amino]benzimidazol-1-yl]-2-pyridyl]-1-methyl-pyrazole-3-carbonitrile (15 mg, 0.030 mmol, 24.3% yield) as a white solid. LC-MS: m/z=450.2 [M+H]⁺, ESI pos.

### Step 2: 5-[3-(1-hydroxyethyl)-6-[5-[(6-methylpyridazin-3-yl)amino]benzimidazol-1-yl]-2-pyridyl]-1-methyl-pyrazole-3-carbonitrile

Error! Objects cannot be created from editing field codes.

Prepared in analogy to example 53, step 4 using 5-[3-acetyl-6-[5-[(6-methylpyridazin-3-yl)amino]benzimidazol-1-yl]-2-pyridyl]-1-methyl-pyrazole-3-carbonitrile (15.0 mg, 0.030 mmol, 1.0 equiv.) at −78° C. to give 5-[3-(1-hydroxyethyl)-6-[5-[(6-methylpyridazin-3-yl)amino]benzimidazol-1-yl]-2-pyridyl]-1-methyl-pyrazole-3-carbonitrile (11.7 mg, 0.030 mmol, 74.39% yield) as a white solid. LC-MS: m/z=452.1 [M+H]⁺, ESI pos. ¹H NMR (400 MHz, CD₃OD) δ ppm 8.95-9.08 (m, 1H) 8.30-8.35 (m, 1H) 8.11-8.19 (m, 2H) 7.97-8.02 (m, 1H) 7.79-7.84 (m, 1H) 7.62-7.68 (m, 1H) 7.40-7.46 (m, 1H) 7.00-7.03 (m, 1H) 4.83-4.86 (m, 1H) 3.83-3.87 (m, 3H) 2.55-2.60 (m, 3H) 1.34-1.39 (m, 3H).

### Example 256

### 2-[3-(1-Hydroxyethyl)-6-[5-[(6-methylpyridazin-3-yl)amino]benzimidazol-1-yl]-2-pyridyl]triazole-4-carbonitrile

Error! Objects cannot be created from editing field codes.

Example 256 was prepared in analogy to example 53, step 4 using 2-[3-acetyl-6-[5-[(6-methylpyridazin-3-yl)amino]benzimidazol-1-yl]-2-pyridyl]triazole-4-carbonitrile (20.0 mg, 0.050 mmol, 1.0 equiv., prepared in example 253, step 1) and NaBH₄ (6.0 mg, 0.160 mmol, 3.5 equiv.) in MeOH/DCM to yield 2-[3-(1-hydroxyethyl)-6-[5-[(6-methylpyridazin-3-yl)amino]benzimidazol-1-yl]-2-pyridyl]triazole-4-carbonitrile (13.7 mg, 0.030 mmol, 68.19% yield) as yellow solid. LC-MS: m/z=439.2[M+H]⁺, ESI pos. ¹H NMR (400 MHz, DMSO-d₆) δ=9.23 (s, 1H), 9.03 (d, J=13.4 Hz, 2H), 8.50 (d, J=8.6 Hz, 1H), 8.41 (d, J=1.9 Hz, 1H), 8.34-8.25 (m, 2H), 7.52 (dd, J=2.0, 8.9 Hz, 1H), 7.33 (d, J=9.1 Hz, 1H), 7.09 (d, J=9.0 Hz, 1H), 5.59 (br s, 1H), 4.96 (q, J=6.3 Hz, 1H), 2.48 (s, 3H), 1.36 (d, J=6.4 Hz, 3H).

### Example 263

### 1-[6-(3-Cyano-5-methyl-pyrazol-1-yl)-5-(1-hydroxyethyl)-2-pyridyl]benzimidazole-5-carbonitrile

Error! Objects cannot be created from editing field codes.

### Step 1: 1-[5-acetyl-6-(3-cyano-5-methyl-pyrazol-1-yl)-2-pyridyl]benzimidazole-5-carbonitrile

Error! Objects cannot be created from editing field codes.

1-[3-acetyl-6-(5-bromobenzimidazol-1-yl)-2-pyridyl]-5-methyl-pyrazole-3-carbonitrile (30 mg, 0.071 mmol, 1 equiv.) was suspended in 1,4-dioxane (1 mL) and (2S)-pyrrolidine-2-carbonitrile; hydrochloride (10.39 mg, 0.078 mmol, 1.1 equiv.), water (33.49 mg, 33.49 μL, 1.86 mmol, 26 equiv.) and Cs₂CO₃ (69.61 mg, 0.214 mmol, 3.0 equiv.) were added at rt. The reaction mixture was degased with argon before QPhosPd(crotyl)Cl (5.17 mg, 0.006 mmol, 0.08 equiv.) was added. The vial was closed and heated to 80° C. overnight. The reaction mixture was diluted with water and extracted twice with EtOAc. The organic layers were dried over MgSO₄ and concentrated to dryness. The crude material was purified by flash column chromatography (silica gel, 0-5% MeOH in DCM) to yield 1-[5-acetyl-6-(3-cyano-5-methyl-pyrazol-1-yl)-2-pyridyl]benzimidazole-5-carbonitrile (17.4 mg, 64.5%) as pink solid. LC-MS: m/z=368.1258 [M+H]⁺, ESI pos.

### Step 2: 1-[6-(3-cyano-5-methyl-pyrazol-1-yl)-5-(-hydroxyethyl)-2-pyridyl]benzimidazole-5-carbonitrile

Error! Objects cannot be created from editing field codes.

Prepared in analogy to example 55, step 3 using 1-[5-acetyl-6-(3-cyano-5-methyl-pyrazol-1-yl)-2-pyridyl]benzimidazole-5-carbonitrile (15 mg, 0.041 mmol, 1.0 equiv.) and NaBH₄ (3.09 mg, 0.082 mmol, 2.0 equiv.) in THF/ⁱPrOH (1:1) at RT to obtain the title compound 1-[6-(3-cyano-5-methyl-pyrazol-1-yl)-5-(1-hydroxyethyl)-2-pyridyl]benzimidazole-5-carbonitrile (10.2 mg, 67.6%) as white solid. LC-MS: m/z=370.20 [M+H]⁺, ESI pos.

### Example 264

### 1-[2-(1-Ethyl-3-methyl-pyrazol-4-yl)-6-[5-[(6-methylpyridazin-3-yl)amino]benzimidazol-1-yl]-3-pyridyl]ethanol; formic acid

Error! Objects cannot be created from editing field codes.

### Step 1: 1-ethyl-3-methyl-4-(4,4,5,5-tetramethyl-1,3,2-dioxaborolan-2-yl)pyrazole and 1-ethyl-5-methyl-4-(4,4,5,5-tetramethyl-1,3,2-dioxaborolan-2-yl)pyrazole

Error! Objects cannot be created from editing field codes.

A mixture of 3-methyl-4-(4,4,5,5-tetramethyl-1,3,2-dioxaborolan-2-yl)-1H-pyrazole (500.0 mg, 2.4 mmol, 1.0 equiv.), iodoethane (0.38 mL, 4.81 mmol, 2.0 equiv.), Cs₂CO₃ (1.57 mg, 4.81 mmol, 2.0 equiv.) and CH₃CN (10 mL) was stirred at 75° C. for 12 h. The reaction mixture was filtered and the mother liquor was concentrated under reduced pressure to give a 1:1 mixture of 1-ethyl-3-methyl-4-(4,4,5,5-tetramethyl-1,3,2-dioxaborolan-2-yl)pyrazole and 1-ethyl-5-methyl-4-(4,4,5,5-tetramethyl-1,3,2-dioxaborolan-2-yl)pyrazole (560 mg, 2.38 mmol, 98.8% yield) as white gum. LC-MS: m/z=237.2 [M+H]⁺, ESI pos.

### Step 2: 1-[2-(-ethyl-3-methyl-pyrazol-4-yl)-6-[5-[(6-methylpyridazin-3-yl)amino]benzimidazol-1-yl]-3-pyridyl]ethanone and 1-[2-(1-ethyl-5-methyl-pyrazol-4-yl)-6-[5-[(6-methylpyridazin-3-yl)amino]benzimidazol-1-yl]-3-pyridyl]ethanone

Error! Objects cannot be created from editing field codes.

Prepared in analogy to example 143, step 2 using 1-[2-chloro-6-[5-[(6-methylpyridazin-3-yl)amino]benzimidazol-1-yl]-3-pyridyl]ethanone (120.0 mg, 0.320 mmol, 1.0 equiv., prepared in example 76, step 2) and 1-ethyl-3-methyl-4-(4,4,5,5-tetramethyl-1,3,2-dioxaborolan-2-yl)pyrazole to give a mixture of 1-[2-(1-ethyl-5-methyl-pyrazol-4-yl)-6-[5-[(6-methylpyridazin-3-yl)amino]benzimidazol-1-yl]-3-pyridyl]ethanone and 1-[2-(1-ethyl-3-methyl-pyrazol-4-yl)-6-[5-[(6-methylpyridazin-3-yl)amino]benzimidazol-1-yl]-3-pyridyl]ethanone (60 mg, 0.14 mmol, 41.8% yield) as yellow solid. LC-MS: m/z=453.0 [M+H]⁺, ESI pos.

### Step 3: 1-[2-(1-ethyl-3-methyl-pyrazol-4-yl)-6-[5-[(6-methylpyridazin-3-yl)amino]benzimidazol-1-yl]-3-pyridyl]ethanol; formic acid

Error! Objects cannot be created from editing field codes.

Prepared in analogy to example 53, step 4 using 1-[2-(1-ethyl-3-methyl-pyrazol-4-yl)-6-[5-[(6-methylpyridazin-3-yl)amino]benzimidazol-1-yl]-3-pyridyl]ethanone (30.0 mg, 0.070 mmol, 1.0 equiv.) and NaBH₄ (12.61 mg, 0.330 mmol, 5.0 equiv.) to yield 1-[2-(1-ethyl-3-methyl-pyrazol-4-yl)-6-[5-[(6-methylpyridazin-3-yl)amino]benzimidazol-1-yl]-3-pyridyl]ethanol; formic acid (18.2 mg, 0.040 mmol, 54.2% yield) after purification by preparative HPLC (Phenomenex Luna C18 150 mm×25 mm×10 μm, gradient 10-30% CH₃CN in H₂O (with 0.225% formic acid) over 10 min, then 100% CH₃CN (2 min), flow rate 25 mL/min, 1 injection). LC-MS: m/z=455.1 [M+H]⁺, ESI pos. ¹H NMR (400 MHz, CD₃OD) δ=8.82 (s, 1H), 8.27 (d, J=8.4 Hz, 1H), 8.23 (br s, 1H), 8.18 (d, J=2.0 Hz, 1H), 8.16 (d, J=9.2 Hz, 1H), 7.87 (s, 1H), 7.80 (d, J=8.4 Hz, 1H), 7.60-7.52 (m, 1H), 7.37 (d, J=9.2 Hz, 1H), 7.15 (d, J=9.2 Hz, 1H), 5.08-5.02 (m, 1H), 4.30-4.15 (m, 2H), 2.53 (s, 3H), 2.34 (s, 3H), 1.52 (t, J=7.2 Hz, 3H), 1.47 (d, J=6.4 Hz, 3H).

### Example 265

### 1-[2-[1-(2-Methoxyethyl)-3-methyl-pyrazol-4-yl]-6-[5-[(6-methylpyridazin-3-yl)amino]benzimidazol-1-yl]-3-pyridyl]ethanol; formic acid

Error! Objects cannot be created from editing field codes.

### Step 1: 1-(2-methoxyethyl)-5-methyl-4-(4,4,5,5-tetramethyl-1,3,2-dioxaborolan-2-yl)pyrazole and 1-(2-methoxyethyl)-3-methyl-4-(4,4,5,5-tetramethyl-1,3,2-dioxaborolan-2-yl)pyrazole

Error! Objects cannot be created from editing field codes.

A mixture of Cs₂CO₃ (1.57 g, 4.81 mmol, 2.0 equiv.), 3-methyl-4-(4,4,5,5-tetramethyl-1,3,2-dioxaborolan-2-yl)-1H-pyrazole (500.0 mg, 2.4 mmol, 1.0 equiv.), 2-bromoethyl methyl ether (0.25 mL, 2.64 mmol, 1.1 equiv.) and CH₃CN (10 mL) was stirred at 80° C. for 12 h. The reaction mixture was filtered and the filter liquor was concentrated under reduced pressure to give a 1:1 mixture of 1-(2-methoxyethyl)-5-methyl-4-(4,4,5,5-tetramethyl-1,3,2-dioxaborolan-2-yl)pyrazole and 1-(2-methoxyethyl)-3-methyl-4-(4,4,5,5-tetramethyl-1,3,2-dioxaborolan-2-yl)pyrazole (600 mg, 2.26 mmol, 93.8% yield) as brown oil. LC-MS: m/z=267.2 [M+H]⁺, ESI pos.

### Step 2: 1-[2-[1-(2-methoxyethyl)-3-methyl-pyrazol-4-yl]-6-[5-[(6-methylpyridazin-3-yl)amino]benzimidazol-1-yl]-3-pyridyl]ethanone and 1-[2-[1-(2-methoxyethyl)-5-methyl-pyrazol-4-yl]-6-[5-[(6-methylpyridazin-3-yl)amino]benzimidazol-1-yl]-3-pyridyl]ethanone

Error! Objects cannot be created from editing field codes.

Prepared in analogy to example 143, step 2 using 1-[2-chloro-6-[5-[(6-methylpyridazin-3-yl)amino]benzimidazol-1-yl]-3-pyridyl]ethanone (150.0 mg, 0.400 mmol, 1.0 equiv., prepared in example 76, step 2) and 1-(2-methoxyethyl)-5-methyl-4-(4,4,5,5-tetramethyl-1,3,2-dioxaborolan-2-yl)pyrazole (158.07 mg, 0.590 mmol, 1.5 equiv.) to yield a 1:1 mixture of 1-[2-[1-(2-methoxyethyl)-5-methyl-pyrazol-4-yl]-6-[5-[(6-methylpyridazin-3-yl)amino]benzimidazol-1-yl]-3-pyridyl]ethanone and 1-[2-[1-(2-methoxyethyl)-3-methyl-pyrazol-4-yl]-6-[5-[(6-methylpyridazin-3-yl)amino]benzimidazol-1-yl]-3-pyridyl]ethanone (80 mg, 0.16 mmol, 41.8% yield) as yellow solid. LC-MS: m/z=483.3 [M+H]⁺, ESI pos.

### Step 3:1-[2-[1-(2-methoxyethyl)-3-methyl-pyrazol-4-yl]-6-[5-[(6-methylpyridazin-3-yl)amino]benzimidazol-1-yl]-3-pyridyl]ethanol; formic acid

Error! Objects cannot be created from editing field codes.

Prepared in analogy to example 53, step 4 using 1-[2-[1-(2-methoxyethyl)-3-methyl-pyrazol-4-yl]-6-[5-[(6-methylpyridazin-3-yl)amino]benzimidazol-1-yl]-3-pyridyl]ethanone (30.0 mg, 0.060 mmol, 1.0 equiv.) and NaBH₄ (11.82 mg, 0.310 mmol, 5.0 equiv.) to yield 1-[2-[1-(2-methoxyethyl)-3-methyl-pyrazol-4-yl]-6-[5-[(6-methylpyridazin-3-yl)amino]benzimidazol-1-yl]-3-pyridyl]ethanol; formic acid (20.6 mg, 0.040 mmol, 68.4% yield) as yellow solid after purification by preparative HPLC (Phenomenex Luna C18 150 mm×25 mm, 10 μm, gradient 20-40% CH₃CN in H₂O (with 0.225% formic acid), flow rate 25 mL/min). LC-MS: m/z=485.1 [M+H]⁺, ESI pos. ¹H NMR (400 MHz, CD₃OD) δ=8.81 (s, 1H), 8.27 (d, J=8.4 Hz, 1H), 8.21-8.09 (m, 2H), 7.87 (s, 1H), 7.79 (d, J=8.4 Hz, 1H), 7.60-7.52 (m, 1H), 7.36 (d, J=9.2 Hz, 1H), 7.14 (d, J=9.2 Hz, 1H), 5.14-5.03 (m, 1H), 4.33 (t, J=5.2 Hz, 2H), 3.78 (t, J=5.2 Hz, 2H), 3.37 (s, 3H), 2.53 (s, 3H), 2.34 (s, 3H), 1.47 (d, J=6.4 Hz, 3H).

### Example 266

### 1-[2-[1-(Cyclopropylmethyl)-3-methyl-pyrazol-4-yl]-6-[5-[(6-methylpyridazin-3-yl)amino]benzimidazol-1-yl]-3-pyridyl]ethanol; formic acid

Error! Objects cannot be created from editing field codes.

### Step 1: 1-(cyclopropylmethyl)-5-methyl-4-(4,4,5,5-tetramethyl-1,3,2-dioxaborolan-2-yl)pyrazole and 1-(cyclopropylmethyl)-3-methyl-4-(4,4,5,5-tetramethyl-1,3,2-dioxaborolan-2-yl)pyrazole

Error! Objects cannot be created from editing field codes.

A mixture of 3-methyl-4-(4,4,5,5-tetramethyl-1,3,2-dioxaborolan-2-yl)-1H-pyrazole (500.0 mg, 2.4 mmol, 1.0 equiv.), (bromomethyl)cyclopropane (0.26 mL, 2.64 mmol, 1.1 equiv.) and Cs₂CO₃ (1.57 mg, 4.81 mmol, 2 equiv.) in CH₃CN (10 mL) was stirred at 80° C. for 12 h. The reaction mixture was filtered and the mother liquor was concentrated under reduced pressure to give a 2:1 mixture of 1-(cyclopropylmethyl)-5-methyl-4-(4,4,5,5-tetramethyl-1,3,2-dioxaborolan-2-yl)pyrazole and 1-(cyclopropylmethyl)-3-methyl-4-(4,4,5,5-tetramethyl-1,3,2-dioxaborolan-2-yl)pyrazole (600 mg, 2.28 mmol, 95.4% yield) as yellow gum. LC-MS: m/z=263.1 [M+H]⁺, ESI pos. ¹H NMR (400 MHz, CDCl₃) δ=7.76 (s, 1H), 7.73 (s, 1H), 3.99 (t, J=6.9 Hz, 4H), 2.50-2.43 (m, 7H), 1.33-1.30 (m, 25H), 0.70-0.62 (m, 3H), 0.61-0.51 (m, 2H), 0.45-0.34 (m, 4H).

### Step 2: 1-[2-[1-(cyclopropylmethyl)-3-methyl-pyrazol-4-yl]-6-[5-[(6-methylpyridazin-3-yl)amino]benzimidazol-1-yl]-3-pyridyl]ethanone and 1-[2-[1-(cyclopropylmethyl)-5-methyl-pyrazol-4-yl]-6-[5-[(6-methylpyridazin-3-yl)amino]benzimidazol-1-yl]-3-pyridyl]ethanone

Error! Objects cannot be created from editing field codes.

Prepared in analogy to example 143, step 2 using 1-(cyclopropylmethyl)-5-methyl-4-(4,4,5,5-tetramethyl-1,3,2-dioxaborolan-2-yl)pyrazole (249.1 mg, 0.950 mmol, 3.0 equiv.) and 1-[2-chloro-6-[5-[(6-methylpyridazin-3-yl)amino]benzimidazol-1-yl]-3-pyridyl]ethanone (120.0 mg, 0.32 mmol, 1.0 equiv to yield a 2:1 mixture of 1-[2-[1-(cyclopropylmethyl)-3-methyl-pyrazol-4-yl]-6-[5-[(6-methylpyridazin-3-yl)amino]benzimidazol-1-yl]-3-pyridyl]ethanone and 1-[2-[1-(cyclopropylmethyl)-5-methyl-pyrazol-4-yl]-6-[5-[(6-methylpyridazin-3-yl)amino]benzimidazol-1-yl]-3-pyridyl]ethanone (69 mg, 0.15 mmol, 45.6% yield) as yellow solid. LC-MS: m/z=479.2 [M+H]⁺, ESI pos.

### Step 3: 1-[2-[1-(cyclopropylmethyl)-3-methyl-pyrazol-4-yl]-6-[5-[(6-methylpyridazin-3-yl)amino]benzimidazol-1-yl]-3-pyridyl]ethanol; formic acid

Error! Objects cannot be created from editing field codes.

Prepared in analogy to example 53, step 4 using 1-[2-[1-(cyclopropylmethyl)-3-methyl-pyrazol-4-yl]-6-[5-[(6-methylpyridazin-3-yl)amino]benzimidazol-1-yl]-3-pyridyl]ethanone (30.0 mg, 0.060 mmol, 1.0 equiv.) in methanol (2 mL) and NaBH₄ (11.92 mg, 0.310 mmol, 5 equiv.) to yield 1-[2-[1-(cyclopropylmethyl)-3-methyl-pyrazol-4-yl]-6-[5-[(6-methylpyridazin-3-yl)amino]benzimidazol-1-yl]-3-pyridyl]ethanol; formic acid (20.6 mg, 0.040 mmol, 68.4% yield) as yellow solid after purification by preparative HPLC (Phenomenex Luna C18 150 mm×25 mm×10 μm, gradient 20-40% CH₃CN in H₂O (with 0.225% formic acid) over 10 min, then 100% CH₃CN (2 min), flow rate 25 mL/min). LC-MS: m/z=481.2 [M+H]⁺, ESI pos. ¹H NMR (400 MHz, CD₃OD) δ=8.82 (s, 1H), 8.27 (d, J=8.4 Hz, 1H), 8.21 (s, 1H), 8.20-8.14 (m, 2H), 7.92 (s, 1H), 7.80 (d, J=8.4 Hz, 1H), 7.60-7.53 (m, 1H), 7.37 (d, J=9.2 Hz, 1H), 7.16 (d, J=9.2 Hz, 1H), 5.09-5.03 (m, 1H), 4.03 (d, J=7.2 Hz, 2H), 2.53 (s, 3H), 2.35 (s, 3H), 1.48 (d, J=6.4 Hz, 3H), 1.42-1.30 (m, 1H), 0.70-0.62 (m, 2H), 0.49-0.39 (m, 2H).

### Example 269

### 5-Methyl-1-[3-methylol-6-[6-[(6-methylpyridazin-3-yl)amino]benzimidazol-1-yl]-2-pyridyl]pyrazole-3-carbonitrile; formic acid

Error! Objects cannot be created from editing field codes.

### Step 1: 1-(6-chloro-3-methylol-2-pyridyl)-5-methyl-pyrazole-3-carbonitrile

Error! Objects cannot be created from editing field codes.

1-(6-chloro-3-formyl-2-pyridyl)-5-methyl-pyrazole-3-carbonitrile (100 mg, 0.405 mmol, 1.0 equiv., prepared in example 243, step 2) was dissolved in MeOH (1 mL). NaBH₄ (757.49 ug, 0.020 mmol, 3.0 equiv.) was added and the reaction mixture was stirred at RT over 30 minutes. Water was added and the product was purified by reverse phase chromatography to yield the title compound (75 mg, 70.7% yield) as white lyophilized powder. LC-MS: m/z=249.1 [M+H]⁺, ESI pos.

### Step 2: 5-methyl-1-[3-methylol-6-[6-[(6-methylpyridazin-3-yl)amino]benzimidazol-1-yl]-2-pyridyl]pyrazole-3-carbonitrile; formic acid and 5-methyl-1-[3-methylol-6-[5-[(6-methylpyridazin-3-yl)amino]benzimidazol-1-yl]-2-pyridyl]pyrazole-3-carbonitrile; formic acid

Error! Objects cannot be created from editing field codes.

Prepared in analogy to example 65, step 2 using 1-(6-chloro-3-methylol-2-pyridyl)-5-methyl-pyrazole-3-carbonitrile (75 mg, 0.302 mmol, 1.0 equiv.) and 1H-benzimidazol-5-yl-(6-methylpyridazin-3-yl)amine (81.52 mg, 0.362 mmol, 1.2 equiv., prepared in example 64, intermediate 1) to yield the title compounds: 5-methyl-1-[3-methylol-6-[6-[(6-methylpyridazin-3-yl)amino]benzimidazol-1-yl]-2-pyridyl]pyrazole-3-carbonitrile; formic acid (3.0 mg, 2.0% yield) and 5-methyl-1-[3-methylol-6-[5-[(6-methylpyridazin-3-yl)amino]benzimidazol-1-yl]-2-pyridyl]pyrazole-3-carbonitrile; formic acid (2.0 mg, 1.4% yield) as light yellow lyophilized powders. Regioisomer 1: LC-MS: m/z=438.3 [M+H]⁺, ESI pos. ¹H NMR (600 MHz, DMSO-d₆) δ=9.26 (s, 1H), 8.88 (s, 1H), 8.83 (d, J=1.8 Hz, 1H), 8.41 (d, J=8.4 Hz, 1H), 8.19 (d, J=8.5 Hz, 1H), 7.68 (d, J=9.1 Hz, 1H), 7.42 (dd, J=8.8, 2.1 Hz, 1H), 7.29 (d, J=9.2 Hz, 1H), 7.06 (d, J=9.0 Hz, 1H), 7.01 (d, J=0.9 Hz, 1H), 5.50 (t, J=5.5 Hz, 1H), 4.42 (d, J=4.9 Hz, 2H), 2.39 (d, J=0.8 Hz, 3H). Regioisomer 2: LC-MS: m/z=438.3 [M+H]⁺, ESI pos. ¹H NMR (600 MHz, DMSO-d₆) δ=9.21 (s, 1H), 9.00 (s, 1H), 8.35-8.46 (m, 1H), 8.31-8.41 (m, 2H), 8.21-8.23 (m, 1H), 8.24 (d, J=8.5 Hz, 1H), 8.05 (d, J=8.8 Hz, 1H), 7.49 (dd, J=8.9, 2.1 Hz, 1H), 7.39-7.53 (m, 1H), 7.33 (d, J=9.1 Hz, 1H), 7.06-7.20 (m, 2H), 5.60 (s, 1H), 4.42 (s, 2H), 2.38 (d, J=0.8 Hz, 3H).

### Example 270

### 5-Methyl-1-[3-methylol-6-[5-[(6-methylpyridazin-3-yl)amino]benzimidazol-1-yl]-2-pyridyl]pyrazole-3-carbonitrile; formic acid

Error! Objects cannot be created from editing field codes.

Prepared in analogy to example 65, step 2 using 1-(6-chloro-3-methylol-2-pyridyl)-5-methyl-pyrazole-3-carbonitrile (75 mg, 0.302 mmol, 1.0 equiv., prepared in example 269, step 1) and 1H-benzimidazol-5-yl-(6-methylpyridazin-3-yl)amine (81.52 mg, 0.362 mmol, 1.2 equiv., prepared in example 64, intermediate 1) to yield the title compound (3.0 mg, 2.0% yield) (2.0 mg, 1.4% yield) as light yellow lyophilized powders. LC-MS: m/z=438.3 [M+H]⁺, ESI pos.

### Example 271

### 1-[2-(1-Ethyl-5-methyl-pyrazol-4-yl)-6-[5-[(6-methylpyridazin-3-yl)amino]benzimidazol-1-yl]-3-pyridyl]ethanol; formic acid

Error! Objects cannot be created from editing field codes.

Prepared in analogy to example 53, step 4 using 1-[2-(1-ethyl-5-methyl-pyrazol-4-yl)-6-[5-[(6-methylpyridazin-3-yl)amino]benzimidazol-1-yl]-3-pyridyl]ethanone (15.0 mg, 0.030 mmol, 1.0 equiv., prepared in example 264, step 2) and NaBH₄ (6.3 mg, 0.170 mmol, 5.0 equiv.) to yield the title compound (6.9 mg, 45.2% yield) as yellow solid. LC-MS: m/z=455.1 [M+H]⁺, ESI pos. ¹H NMR (400 MHz, CD₃OD) δ=8.82 (s, 1H), 8.28 (d, J=8.4 Hz, 2H), 8.20-8.11 (m, 2H), 7.81 (d, J=8.4 Hz, 1H), 7.69 (s, 1H), 7.62-7.55 (m, 1H), 7.36 (d, J=9.2 Hz, 1H), 7.14 (d, J=9.2 Hz, 1H), 5.15-5.06 (m, 1H), 4.30-4.19 (m, 2H), 2.53 (s, 3H), 2.43 (s, 3H), 1.50-1.44 (m, 6H).

### Example 272

### 1-[2-[1-(Cyclopropylmethyl)-5-methyl-pyrazol-4-yl]-6-[5-[(6-methylpyridazin-3-yl)amino]benzimidazol-1-yl]-3-pyridyl]ethanol; formic acid

Error! Objects cannot be created from editing field codes.

Prepared in analogy to example 53, step 4 using 1-[2-[1-(cyclopropylmethyl)-5-methyl-pyrazol-4-yl]-6-[5-[(6-methylpyridazin-3-yl)amino]benzimidazol-1-yl]-3-pyridyl]ethanone (15.0 mg, 0.030 mmol, 1.0 equiv., prepared in example 266, step 2) and NaBH₄ (5.96 mg, 0.160 mmol, 5.0 equiv.) to yield 1-[2-[1-(cyclopropylmethyl)-5-methyl-pyrazol-4-yl]-6-[5-[(6-methylpyridazin-3-yl)amino]benzimidazol-1-yl]-3-pyridyl]ethanol; formic acid (9.2 mg, 0.020 mmol, 61.1% yield) as yellow solid. LC-MS: m/z=481.0 [M+H]⁺, ESI pos. ¹H NMR (400 MHz, CD₃OD) δ=8.83 (s, 1H), 8.29 (d, J=8.4 Hz, 1H), 8.27-8.23 (m, 1H), 8.17 (d, J=2.0 Hz, 1H), 8.15 (d, J=8.8 Hz, 1H), 7.82 (d, J=8.4 Hz, 1H), 7.69 (s, 1H), 7.61-7.55 (m, 1H), 7.37 (d, J=9.2 Hz, 1H), 7.14 (d, J=9.2 Hz, 1H), 5.14-5.08 (m, 1H), 4.10 (d, J=6.8 Hz, 2H), 2.53 (s, 3H), 2.44 (s, 3H), 1.47 (d, J=6.4 Hz, 3H), 1.40-1.26 (m, 1H), 0.68-0.57 (m, 2H), 0.50-0.42 (m, 2H).

### Example 273

### 1-[2-[1-(Cyclobutylmethyl)-5-methyl-pyrazol-4-yl]-6-[5-[(6-methylpyridazin-3-yl)amino]benzimidazol-1-yl]-3-pyridyl]ethanol; formic acid

Error! Objects cannot be created from editing field codes.

### Step 1: 1-(cyclobutylmethyl)-3-methyl-4-(4,4,5,5-tetramethyl-1,3,2-dioxaborolan-2-yl)pyrazole and 1-(cyclobutylmethyl)-5-methyl-4-(4,4,5,5-tetramethyl-1,3,2-dioxaborolan-2-yl)pyrazole

Error! Objects cannot be created from editing field codes.

Prepared in analogy to example 264, step 1 using 3-methyl-4-(4,4,5,5-tetramethyl-1,3,2-dioxaborolan-2-yl)-1H-pyrazole (500.0 mg, 2.4 mmol, 1.0 equiv.) and (bromomethyl)cyclobutane (393.94 mg, 2.64 mmol, 1.1 equiv.) to yield a 1:1 mixture of 1-(cyclobutylmethyl)-3-methyl-4-(4,4,5,5-tetramethyl-1,3,2-dioxaborolan-2-yl)pyrazole and 1-(cyclobutylmethyl)-5-methyl-4-(4,4,5,5-tetramethyl-1,3,2-dioxaborolan-2-yl)pyrazole (590 mg, 1.07 mmol, 88.9% yield) as a yellow oil. LC-MS: m/z=277.2 [M+H]⁺, ESI pos.

### Step 2: 1-[2-[1-(cyclobutylmethyl)-3-methyl-pyrazol-4-yl]-6-[5-[(6-methylpyridazin-3-yl)amino]benzimidazol-1-yl]-3-pyridyl]ethanone and 1-[2-[1-(cyclobutylmethyl)-5-methyl-pyrazol-4-yl]-6-[5-[(6-methylpyridazin-3-yl)amino]benzimidazol-1-yl]-3-pyridyl]ethanone

Error! Objects cannot be created from editing field codes.

Prepared in analogy to example 143, step 2 using 1-[2-chloro-6-[5-[(6-methylpyridazin-3-yl)amino]benzimidazol-1-yl]-3-pyridyl]ethanone (150.0 mg, 0.400 mmol, 1.0 equiv., prepared in example 76, step 2) and 1-(cyclobutylmethyl)-3-methyl-4-(4,4,5,5-tetramethyl-1,3,2-dioxaborolan-2-yl)pyrazole (328.07 mg, 1.19 mmol, 3.0 equiv.), to yield 1-[2-[1-(cyclobutylmethyl)-3-methyl-pyrazol-4-yl]-6-[5-[(6-methylpyridazin-3-yl)amino]benzimidazol-1-yl]-3-pyridyl]ethanone (35 mg, 17.9% yield) as a yellow solid and 1-[2-[1-(cyclobutylmethyl)-5-methyl-pyrazol-4-yl]-6-[5-[(6-methylpyridazin-3-yl)amino]benzimidazol-1-yl]-3-pyridyl]ethanone (25 mg, 12.8% yield) as an orange solid after separation by SFC (Chiralpak AS-3 250 mm×30 mm, 10 μm, MeOH (with 0.1% ammonium hydroxide)). Regioisomer 1: LC-MS: m/z=493.1 [M+H]⁺, ESI pos. ¹H NMR (400 MHz, DMSO-d₆) δ=9.23 (s, 1H), 9.02 (s, 1H), 8.44 (d, J=2.0 Hz, 1H), 8.25 (d, J=8.8 Hz, 1H), 8.19 (d, J=8.6 Hz, 1H), 7.93 (s, 1H), 7.91 (s, 1H), 7.50 (dd, J=2.0, 8.9 Hz, 1H), 7.33 (d, J=9.2 Hz, 1H), 7.08 (d, J=9.0 Hz, 1H), 4.14 (d, J=7.3 Hz, 2H), 2.83-2.74 (m, 1H), 2.48 (s, 3H), 2.32 (s, 3H), 2.27 (s, 3H), 2.04-1.95 (m, 2H), 1.90-1.76 (m, 4H). Regioisomer 2: LC-MS: m/z=493.1 [M+H]⁺, ESI pos. ¹H NMR (400 MHz, DMSO-d₆) δ=9.23 (s, 1H), 9.04 (s, 1H), 8.43 (d, J=1.8 Hz, 1H), 8.26 (d, J=8.8 Hz, 1H), 8.21 (d, J=8.4 Hz, 1H), 7.93 (d, J=8.4 Hz, 1H), 7.56 (s, 1H), 7.51 (dd, J=2.0, 8.9 Hz, 1H), 7.33 (d, J=9.0 Hz, 1H), 7.08 (d, J=9.2 Hz, 1H), 4.17 (d, J=7.1 Hz, 2H), 2.86-2.77 (m, 1H), 2.48 (s, 3H), 2.39 (s, 3H), 2.33 (s, 3H), 2.02-1.94 (m, 2H), 1.89-1.79 (m, 4H).

### Step 3: 1-[2-[1-(cyclobutylmethyl)-5-methyl-pyrazol-4-yl]-6-[5-[(6-methylpyridazin-3-yl)amino]benzimidazol-1-yl]-3-pyridyl]ethanol; formic acid

Error! Objects cannot be created from editing field codes.

Prepared in analogy to example 55, step 5 using 1-[2-[1-(cyclobutylmethyl)-5-methyl-pyrazol-4-yl]-6-[5-[(6-methylpyridazin-3-yl)amino]benzimidazol-1-yl]-3-pyridyl]ethanone (25.0 mg, 0.050 mmol, 1.0 equiv.) and NaBH₄ (9.6 mg, 0.250 mmol, 5.0 equiv.) to yield 1-[2-[1-(cyclobutylmethyl)-5-methyl-pyrazol-4-yl]-6-[5-[(6-methylpyridazin-3-yl)amino]benzimidazol-1-yl]-3-pyridyl]ethanol; formic acid (23.3 mg, 0.040 mmol, 84.92% yield) as a yellow solid LC-MS: m/z=495.0 [M+H]⁺, ESI pos. ¹H NMR (400 MHz, CD₃OD) δ=8.83 (s, 1H), 8.28 (d, J=8.6 Hz, 1H), 8.21 (s, 1H), 8.18 (d, J=2.0 Hz, 1H), 8.15 (d, J=8.8 Hz, 1H), 7.81 (d, J=8.4 Hz, 1H), 7.68 (s, 1H), 7.57 (dd, J=2.0, 8.8 Hz, 1H), 7.38 (d, J=9.2 Hz, 1H), 7.15 (d, J=9.2 Hz, 1H), 5.09 (q, J=6.4 Hz, 1H), 4.22 (d, J=7.2 Hz, 2H), 2.90 (td, J=7.6, 14.8 Hz, 1H), 2.53 (s, 3H), 2.42 (s, 3H), 2.13-2.05 (m, 2H), 1.98-1.86 (m, 4H), 1.45 (d, J=6.4 Hz, 3H).

### Example 274

### 1-[2-[1-(Cyclobutylmethyl)-3-methyl-pyrazol-4-yl]-6-[5-[(6-methylpyridazin-3-yl)amino]benzimidazol-1-yl]-3-pyridyl]ethanol

Error! Objects cannot be created from editing field codes.

Prepared in analogy to example 55, step 5 using 1-[2-[1-(cyclobutylmethyl)-3-methyl-pyrazol-4-yl]-6-[5-[(6-methylpyridazin-3-yl)amino]benzimidazol-1-yl]-3-pyridyl]ethanone (33.0 mg, 0.070 mmol, 1.0 equiv., prepared in example 273, step 2) to yield 1-[2-[1-(cyclobutylmethyl)-3-methyl-pyrazol-4-yl]-6-[5-[(6-methylpyridazin-3-yl)amino]benzimidazol-1-yl]-3-pyridyl]ethanol (20.8 mg, 0.040 mmol, 61.5% yield) as a yellow solid. LC-MS: m/z=495.0 [M+H]⁺, ESI pos. ¹H NMR (400 MHz, CD₃OD) δ=8.82 (s, 1H), 8.27 (d, J=8.4 Hz, 1H), 8.19 (d, J=2.0 Hz, 1H), 8.16 (d, J=8.9 Hz, 1H), 7.85 (s, 1H), 7.80 (d, J=8.4 Hz, 1H), 7.57 (dd, J=2.0, 8.9 Hz, 1H), 7.37 (d, J=9.2 Hz, 1H), 7.14 (d, J=9.0 Hz, 1H), 5.04 (q, J=6.4 Hz, 1H), 4.18 (d, J=7.3 Hz, 2H), 2.89 (td, J=7.5, 15.2 Hz, 1H), 2.53 (s, 3H), 2.33 (s, 3H), 2.15-2.07 (m, 2H), 1.99-1.84 (m, 4H), 1.46 (d, J=6.5 Hz, 3H).

### Example 276

### 1-[3-[(1S)-1-hydroxyethyl]-6-[5-[(6-methylpyridazin-3-yl)amino]benzimidazol-1-yl]-2-pyridyl]-5-methyl-pyrazole-3-carboximidic acid methyl ester

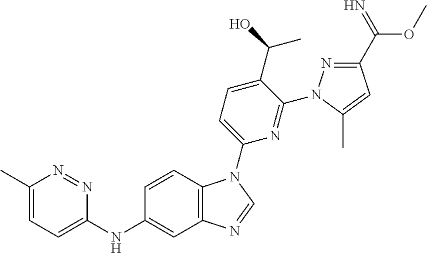

Example 276 was isolated as a by-product during the purification of Example 109 and Example 110 (22 mg, 6% yield) as a white powder. LC-MS: m/z=484.2 [M+H]⁺, ESI pos. ¹H NMR (600 MHz, CDCl₃) δ ppm 8.59 (s, 1H), 8.34 (d, J=8.4 Hz, 1H), 8.02-8.24 (m, 1H), 8.01 (d, J=8.8 Hz, 1H), 7.77 (d, J=1.9 Hz, 1H), 7.72 (d, J=8.4 Hz, 1H), 7.37 (dd, J=8.7, 2.2 Hz, 1H), 7.12-7.15 (m, 1H), 7.06-7.09 (m, 1H), 6.96 (br d, J=1.1 Hz, 1H), 6.60 (s, 1H), 4.90 (q, J=6.5 Hz, 1H), 3.98-4.21 (m, 1H), 3.97 (s, 3H), 2.61 (s, 3H), 2.49 (d, J=0.7 Hz, 3H), 1.51-1.53 (m, 3H).

### Example 280

### 1-[3-[(1S)-1-hydroxyethyl]-6-[6-[(6-methylpyridazin-3-yl)amino]benzimidazol-1-yl]-2-pyridyl]-5-methyl-pyrazole-3-carbonitrile

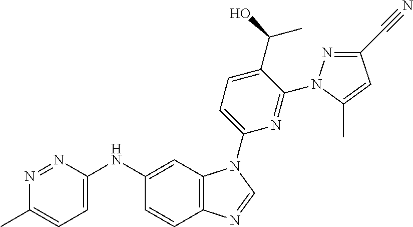 [SMILES: Cc1ccc(Nc2ccc3ncn(-c4ccc([C@H](C)O)c(-n5nc(C#N)cc5C)n4)c3c2)nn1]

Example 280 was isolated as a regioisomer during the preparation of Example 109 and Example 110 (28 mg, 7% yield) as a white powder. LC-MS: m/z=452.3 [M+H]⁺, ESI pos. ¹H NMR (600 MHz, CDCl₃) δ ppm 8.50 (s, 1H), 8.38 (d, J=8.4 Hz, 1H), 8.35 (d, J=1.7 Hz, 1H), 7.78 (dd, J=8.3, 7.2 Hz, 2H), 7.19 (dd, J=8.7, 2.1 Hz, 1H), 7.12 (d, J=9.2 Hz, 2H), 6.99 (d, J=9.2 Hz, 1H), 6.65 (s, 1H), 5.30 (s, 1H), 4.84 (d, J=6.5 Hz, 1H), 2.61 (s, 3H), 2.44 (d, J=0.8 Hz, 3H), 1.47-1.53 (m, 4H), 1.23-1.26 (m, 1H).

### Example 304

### 1-[3-(1-hydroxyethyl)-6-[5-[[6-[1-(oxetan-3-yl)pyrrolidin-2-yl]pyridazin-3-yl]amino]benzimidazol-1-yl]-2-pyridyl]-5-methyl-pyrazole-3-carbonitrile

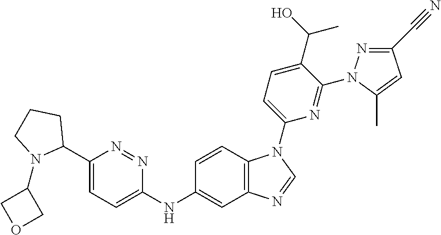 [SMILES: Cc1cc(C#N)nn1-c1nc(-n2cnc3cc(Nc4ccc(C5CCCN5C5COC5)nn4)ccc32)ccc1C(C)O]

### Step 1: tert-butyl 2-(6-aminopyridazin-3-yl)pyrrolidine-1-carboxylate

An oven-dried 15 mL vial equipped with magnetic stir bar was charged with 6-iodopyridazin-3-amine (1.00 g, 4.52 mmol, 1.0 eq), 1-(tert-butoxycarbonyl)pyrrolidine-2-carboxylic acid (1.27 g, 5.88 mmol, 1.3 eq), Ir[dF(CF₃)ppy]2(dtbpy)(PF₆) (51 mg, 0.05 mmol, 0.01 eq), NiCl₂.dtbbpy (90 mg, 0.23 mmol, 0.05 eq), Cs₂CO₃ (2.21 g, 6.79 mmol, 1.5 eq) in DMA (40 mL). The reaction mixture was bubbled with N₂ for 10 minutes then irradiated with two 34 W blue LED lamps (approximately 7 cm away from the light source to keep the reaction temperature at 25° C.) for 12 hours. The reaction mixture was poured into H₂O (150 mL) and extracted with EtOAc (3×40 mL). The combined organic layers were dried over Na₂SO₄, filtered and concentracted. The residue was purified by preparative HPLC (Waters Xbridge 150 mm×25 mm×5 μm, water (10 mM NH₄HCO₃)-ACN) to give tert-butyl 2-(6-aminopyridazin-3-yl)pyrrolidine-1-carboxylate (120 mg, 0.45 mmol, 10% yield) as white solid. LC-MS: m/z=265.1 [M+H]⁺, ESI pos.

### Step 2: tert-butyl 2-[6-[[1-[5-acetyl-6-(3-cyano-5-methyl-pyrazol-1-yl)-2-pyridyl]benzimidazol-5-yl]amino]pyridazin-3-yl]pyrrolidine-1-carboxylate

To a solution of tert-butyl 2-(6-aminopyridazin-3-yl)pyrrolidine-1-carboxylate (69 mg, 0.26 mmol, 1.1 eq) and 1-[3-acetyl-6-(5-bromobenzimidazol-1-yl)-2-pyridyl]-5-methyl-pyrazole-3-carbonitrile (100 mg, 0.24 mmol, 1.0 eq, prepared in example 65, step 1) in 1,4-dioxane (8 mL) was added Cs₂CO₃ (232 mg, 0.71 mmol, 3.0 eq). The mixture was bubbled with N₂ for 10 minutes and [tBuBrettPhos Pd(allyl)]OTf (19 mg, 0.02 mmol, 0.1 eq) was added. The reaction mixture was stirred at 80° C. for 2 hours. The mixture was cooled to RT, poured into H₂O (20 mL) and extracted with EtOAc (3×20 ml). The combined organic layers were washed with brine (20 mL), dried over Na₂SO₄, filtered and concentrated.

The residue was purified by preparative TLC (DCM:MeOH 10:1) to give tert-butyl 2-[6-[[1-[5-acetyl-6-(3-cyano-5-methyl-pyrazol-1-yl)-2-pyridyl]benzimidazol-5-yl]amino]pyridazin-3-yl]pyrrolidine-1-carboxylate (110 mg, 0.18 mmol, 77% yield) as light brown solid. LC-MS: m/z=605.2 [M+H]⁺, ESI pos.

### Step 3: 1-[3-acetyl-6-[5-[(6-pyrrolidin-2-ylpyridazin-3-yl)amino]benzimidazol-1-yl]-2-pyridyl]-5-methyl-pyrazole-3-carbonitrile

Error! Objects cannot be created from editing field codes.

To a solution of tert-butyl 2-[6-[[1-[5-acetyl-6-(3-cyano-5-methyl-pyrazol-1-yl)-2-pyridyl]benzimidazol-5-yl]amino]pyridazin-3-yl]pyrrolidine-1-carboxylate (30 mg, 0.05 mmol, 1.0 eq) in DCM (1.5 mL) was added HCl (4 M in dioxane) (0.8 mL). The mixture was stirred at RT for 1 hour. The reaction mixture was concentrated. The residue was purified by preparative HPLC (Phenomenex Synergi C18 150 mm×25 mm×10 μm, water with FA-ACN) to give 1-[3-acetyl-6-[5-[(6-pyrrolidin-2-ylpyridazin-3-yl)amino]benzimidazol-1-yl]-2-pyridyl]-5-methyl-pyrazole-3-carbonitrile (20 mg, 0.04 mmol, 78% yield) as an off white solid. LC-MS: m/z=505.2 [M+H]⁺, ESI pos.

### Step 4: 1-[3-acetyl-6-[5-[[6-[1-(oxetan-3-yl)pyrrolidin-2-yl]pyridazin-3-yl]amino]benzimidazol-1-yl]-2-pyridyl]-5-methyl-pyrazole-3-carbonitrile

A solution of NaBH₃CN (9 mg, 0.14 mmol, 3.02 eq) and 3-oxetanone (11 mg, 0.15 mmol, 3.16 eq) in MeOH (2 mL) was stirred at 30° C. for 0.5 hour. 1-[3-acetyl-6-[5-[(6-pyrrolidin-2-ylpyridazin-3-yl)amino]benzimidazol-1-yl]-2-pyridyl]-5-methyl-pyrazole-3-carbonitrile; hydrochloride (25 mg, 0.05 mmol, 1.0 eq) was added and the mixture was stirred at 30° C. for 12 hours. The reaction mixture was concentracted. The residue was purified by preparative TLC (DCM:MeOH 10:1) to give 1-[3-acetyl-6-[5-[[6-[1-(oxetan-3-yl)pyrrolidin-2-yl]pyridazin-3-yl]amino]benzimidazol-1-yl]-2-pyridyl]-5-methyl pyrazole-3-carbonitrile (20 mg, 0.04 mmol, 77% yield) as light yellow solid. LC-MS: m/z=561.2 [M+H]⁺, ESI pos.

### Step 5: 1-[3-(1-hydroxyethyl)-6-[5-[[6-[1-(oxetan-3-yl)pyrrolidin-2-yl]pyridazin-3-yl]amino]benzimidazol-1-yl]-2-pyridyl]-5-methyl-pyrazole-3-carbonitrile

To a solution of 1-[3-acetyl-6-[5-[[6-[1-(oxetan-3-yl)pyrrolidin-2-yl]pyridazin-3-yl]amino]benzimidazol-1-yl]-2-pyridyl]-5-methyl-pyrazole-3-carbonitrile (20 mg, 0.04 mmol, 1.0 eq) in MeOH (2.5 mL) was added sodium borohydride (4 mg, 0.11 mmol, 3.0 eq) at 0° C. The reaction mixture was stirred at 0° C. for 1 hour. The reaction mixture was diluted with sat. aq. NH₄Cl (5 mL) at 0° C. and extracted with EtOAc (2×10 ml). The combined organic layers were dried over Na₂SO₄, filtered and concentracted.

The residue was purified by preparative HPLC (Phenomenex Synergi C18 150 mm×25 mm×10 μm, water with FA-ACN) to give 1-[3-(1-hydroxyethyl)-6-[5-[[6-[1-(oxetan-3-yl)pyrrolidin-2-yl]pyridazin-3-yl]amino]benzimidazol-1-yl]-2-pyridyl]-5-methyl-pyrazole-3-carbonitrile (5 mg, 0.01 mmol, 22% yield) as an off white solid. LC-MS: m/z=563.2 [M+H]⁺, ESI pos. ¹H NMR (400 MHz, METHANOL-d4) δ=9.22 (s, 1H), 8.75 (d, J=5.0 Hz, 1H), 8.58 (d, J=8.4 Hz, 1H), 8.36-8.31 (m, 1H), 8.27-8.22 (m, 2H), 8.05-8.01 (m, 1H), 7.59-7.55 (m, 1H), 6.91-6.89 (m, 1H), 4.84-4.77 (m, 2H), 4.71-4.67 (m, 1H), 4.55 (t, J=6.4 Hz, 1H), 4.16-4.08 (m, 1H), 2.93-2.65 (m, 2H), 2.44-2.33 (m, 5H), 2.13-1.98 (m, 4H), 1.45-1.42 (m, 3H).

### Examples 277-279, 281, 283-285, 289, 313, 315, 316, 332, 335, 346, 349, 356, 363, 386, 396, 399

In analogy to Example 76, Examples in the following table were prepared, using the respective building blocks A.X (A.1 to A.20) and B.X (B.1 to B.3) in step 3.

### Building block A.1: 1-(2,2-difluorocyclopropyl)-5-methyl-4-(4,4,5,5-tetramethyl-1,3,2-dioxaborolan-2-yl)pyrazole and 1-(2,2-difluorocyclopropyl)-3-methyl-4-(4,4,5,5-tetramethyl-1,3,2-dioxaborolan-2-yl)pyrazole

Error! Objects cannot be created from editing field codes. Error! Objects cannot be created from editing field codes.

A mixture of 2-bromo-1,1-difluoro-cyclopropane (151 mg, 0.96 mmol, 2.0 equiv.), 3-methyl-4-(4,4,5,5-tetramethyl-1,3,2-dioxaborolan-2-yl)-1H-pyrazole (100 mg, 0.48 mmol, 1.0 equiv.), Cs₂CO₃ (313 mg, 0.96 mmol, 2.0 equiv.) and DMF (2 mL) was stirred at 120° C. for 12 hours. The reaction mixture was cooled to RT, diluted with water (10 mL) and extracted with EtOAC (3×10 mL). The combined organic layers were washed with brine (15 mL), dried over anhydrous Na₂SO₄, filtered and concentrated. The crude was purified by flash chromatography (SiO₂, 10% EtOAc in petroleum ether) to give a mixture of 1-(2,2-difluorocyclopropyl)-5-methyl-4-(4,4,5,5-tetramethyl-1,3,2-dioxaborolan-2-yl)pyrazole and 1-(2,2-difluorocyclopropyl)-3-methyl-4-(4,4,5,5-tetramethyl-1,3,2-dioxaborolan-2-yl)pyrazole (120 mg, 0.42 mmol, 89% yield) as a yellow oil. LC-MS: m/z=284.8 [M+H]⁺, ESI pos.

### Building blocks A.2 and A.3: 1-(2-methoxypropyl)-5-methyl-4-(4,4,5,5-tetramethyl-1,3,2-dioxaborolan-2-yl)pyrazole and 1-(2-methoxypropyl)-3-methyl-4-(4,4,5,5-tetramethyl-1,3,2-dioxaborolan-2-yl)pyrazole

Error! Objects cannot be created from editing field codes. Error! Objects cannot be created from editing field codes.

### Step 1: 1-(4-bromo-5-methyl-pyrazol-1-yl)propan-2-ol and 1-(4-bromo-3-methyl-pyrazol-1-yl)propan-2-ol

To a solution of 4-bromo-3-methylpyrazole (4.0 g, 24.84 mmol, 1.0 equiv.) in DMF (50 mL) was added 1-chloro-2-propanol (3.52 g, 37.27 mmol, 1.5 equiv.) and Cs₂CO₃ (16.19 g, 49.69 mmol, 2.0 equiv.). The mixture was stirred at 100° C. for 12 hours. The mixture was diluted with water (100 mL), extracted with EtOAc (2×100 mL). The combined organic layers were washed with brine, dried over with anhydrous Na₂SO₄ and concentrated under reduced pressure to give a mixture of 1-(4-bromo-5-methyl-pyrazol-1-yl)propan-2-ol and 1-(4-bromo-3-methyl-pyrazol-1-yl)propan-2-ol (5.0 g, 22.82 mmol, 83% yield) as yellow oil which was used without further purification. LC-MS: m/z=219.0 [M+H]⁺, ESI pos.

### Step 2: 4-bromo-1-(2-methoxypropyl)-5-methyl-pyrazole and 4-bromo-1-(2-methoxypropyl)-3-methyl-pyrazole

To a solution of 1-(4-bromo-3-methyl-pyrazol-1-yl)propan-2-ol (1.0 g, 4.56 mmol, 1.0 equiv.) in DMF (10 mL) was added NaH (365 mg, 9.13 mmol, 2.0 equiv.) at 0° C. The mixture was stirred at 0° C. for 30 minutes. Iodomethane (1.42 mL, 22.82 mmol, 5.0 equiv.) was added. The mixture was stirred at 20° C. for 0.5 h. The mixture was diluted with water (50 mL), extracted with EtOAc (3×50 mL). The combined organic layers were washed with brine, dried over with Na₂SO₄ and concentrated under reduced pressure to give a mixture of 4-bromo-1-(2-methoxypropyl)-5-methyl-pyrazole and 4-bromo-1-(2-methoxypropyl)-3-methyl-pyrazole (1.2 g, 5.15 mmol, 96% yield) as yellow oil which was used without further purification. LC-MS: m/z=235.3 [M+H]⁺, ESI pos.

### Step 3: 1-(2-methoxypropyl)-5-methyl-4-(4,4,5,5-tetramethyl-1,3,2-dioxaborolan-2-yl)pyrazole and 1-(2-methoxypropyl)-3-methyl-4-(4,4,5,5-tetramethyl-1,3,2-dioxaborolan-2-yl)pyrazole

To a solution of bis(pinacolato)diboron (2.72 g, 10.72 mmol, 2.5 equiv.) and a mixture of 4-bromo-1-(2-methoxypropyl)-5-methyl-pyrazole and 4-bromo-1-(2-methoxypropyl)-3-methyl-pyrazole (1.0 g, 4.29 mmol, 1.0 equiv.) in di(ethylene glycol)dimethyl ether (10 mL) was added tricyclohexyl phosphine (241 mg, 0.86 mmol, 0.2 equiv.) and water (0.050 mL). The mixture was bubbled with N₂ for about 10 minutes. Pd(II)(OAc)₂ (1.5 mg, 0.010 mmol, 0.10 equiv.) was added and the mixture was stirred under N₂ at 100° C. for 3 hours. The crude was purified by flash chromatography (SiO₂, 10% EtOAc in petroleum ether) to give a mixture of 1-(2-methoxypropyl)-5-methyl-4-(4,4,5,5-tetramethyl-1,3,2-dioxaborolan-2-yl)pyrazole and 1-(2-methoxypropyl)-3-methyl-4-(4,4,5,5-tetramethyl-1,3,2-dioxaborolan-2-yl)pyrazole (880 mg, 3.14 mmol, 36% yield) as brown oil. LC-MS: m/z=281.3 [M+H]⁺, ESI pos.

### Building blocks A.4 and A.6: 1-(2,2-difluoropropyl)-3-methyl-4-(4,4,5,5-tetramethyl-1,3,2-dioxaborolan-2-yl)pyrazole and 1-(2,2-difluoropropyl)-5-methyl-4-(4,4,5,5-tetramethyl-1,3,2-dioxaborolan-2-yl)pyrazole

Error! Objects cannot be created from editing field codes. Error! Objects cannot be created from editing field codes.

Building blocks AA and A.6 was synthesized in analogy to Building block A.1 using 2,2-difluoropropyl trifluoromethanesulfonate to give a mixture of 1-(2,2-difluoropropyl)-3-methyl-4-(4,4,5,5-tetramethyl-1,3,2-dioxaborolan-2-yl)pyrazole and 1-(2,2-difluoropropyl)-5-methyl-4-(4,4,5,5-tetramethyl-1,3,2-dioxaborolan-2-yl)pyrazole which was used without further purification. LC-MS: m/z=287.2 [M+H]⁺, ESI pos.

### Building block A.5: 2-ethyl-5-methyl-4-(4,4,5,5-tetramethyl-1,3,2-dioxaborolan-2-yl)pyridine

Error! Objects cannot be created from editing field codes.

### CAS: 2223045-66-5

### Building block A.7: 2-methoxy-5-methyl-4-(4,4,5,5-tetramethyl-1,3,2-dioxaborolan-2-yl)pyridine

Error! Objects cannot be created from editing field codes.

### CAS: 2246720-33-0

### Building block A.8: 2-cyclopropyl-5-methyl-4-(4,4,5,5-tetramethyl-1,3,2-dioxaborolan-2-yl)pyridine

Error! Objects cannot be created from editing field codes.

### CAS: 2223028-93-9

### Building block A.9: 1-(2,2-difluoroethyl)-3-methyl-4-(4,4,5,5-tetramethyl-1,3,2-dioxaborolan-2-yl)pyrazole

Error! Objects cannot be created from editing field codes.

Building block A.9 was synthesized in analogy to Building block A.1 using 2,2-difluoroethyl trifluoromethanesulfonate to give 1-(2,2-difluoroethyl)-3-methyl-4-(4,4,5,5-tetramethyl-1,3,2-dioxaborolan-2-yl)pyrazole. LC-MS: m/z=273.4 [M+H]⁺, ESI pos.

### Building blocks A.10 and A.11: 3-methyl-4-(4,4,5,5-tetramethyl-1,3,2-dioxaborolan-2-yl)-1-(2,2,2-trifluoroethyl)pyrazole and 5-methyl-4-(4,4,5,5-tetramethyl-1,3,2-dioxaborolan-2-yl)-1-(2,2,2-trifluoroethyl)pyrazole

Error! Objects cannot be created from editing field codes. Error! Objects cannot be created from editing field codes.

### Step 1: 4-bromo-3-methyl-1-(2,2,2-trifluoroethyl)pyrazole and 4-bromo-5-methyl-1-(2,2,2-trifluoroethyl)pyrazole

A mixture of 4-bromo-3-methylpyrazole (15.0 g, 93.17 mmol, 1.0 equiv.), 2,2,2-trifluoroethyl trifluoromethanesulfonate (22.71 g, 97.83 mmol, 1.05 equiv.) and Cs₂CO₃ (25.33 g, 186.34 mmol, 2.0 equiv.) in DMF (150 mL) was stirred at 100° C. for 12 hours. The reaction mixture was cooled and filtered. The filtrate was diluted with H₂O (250 mL) and extracted with EtOAc (3×250 mL). The combined organic layers were washed with brine (250 mL), dried over anhydrous Na₂SO₄, filtered and concentrated. The crude was purified by flash chromatography (SiO₂, 0-20% EtOAc in petroleum ether) to give a mixture of 4-bromo-3-methyl-1-(2,2,2-trifluoroethyl)pyrazole and 4-bromo-5-methyl-1-(2,2,2-trifluoroethyl)pyrazole (19.5 g, 80.24 mmol, 86% yield) as colorless oil which was used without further purification. LC-MS: m/z=242.9 [M+H]⁺, ESI pos.

### Step 2: 3-methyl-4-(4,4,5,5-tetramethyl-1,3,2-dioxaborolan-2-yl)-1-(2,2,2-trifluoroethyl)pyrazole and 5-methyl-4-(4,4,5,5-tetramethyl-1,3,2-dioxaborolan-2-yl)-1-(2,2,2-trifluoroethyl)pyrazole

To a mixture of 4-bromo-3-methyl-1-(2,2,2-trifluoroethyl)pyrazole and 4-bromo-5-methyl-1-(2,2,2-trifluoroethyl)pyrazole (11.0 g, 45.26 mmol, 1.0 equiv.), KOAc (8.88 g, 90.53 mmol, 2.0 equiv.) and bis(pinacolato)diboron (13.79 g, 54.32 mmol, 1.2 equiv.) in 1,4-dioxane (200 mL) was added Pd(dppf)Cl₂.CH₂Cl₂ (3.7 g, 4.53 mmol, 0.1 equiv.). The mixture was stirred at 100° C. for 16 hours under N₂. The mixture was cooled and concentrated under reduced pressure. The residue was purified by flash chromatography (SiO₂, 10-20% EtOAc in petroleum ether) to give a mixture of 3-methyl-4-(4,4,5,5-tetramethyl-1,3,2-dioxaborolan-2-yl)-1-(2,2,2-trifluoroethyl)pyrazole and 5-methyl-4-(4,4,5,5-tetramethyl-1,3,2-dioxaborolan-2-yl)-1-(2,2,2-trifluoroethyl)pyrazole (9.0 g, 31 mmol, 69% yield) as yellow gum. LC-MS: m/z=291.1 [M+H]⁺, ESI pos.

### Building block A.12: 5-methyl-4-(4,4,5,5-tetramethyl-1,3,2-dioxaborolan-2-yl)-2-(trifluoromethyl)pyridine

Error! Objects cannot be created from editing field codes.

### CAS: 2170501-97-8

### Building block A.13: 2-(difluoromethyl)-5-methyl-4-(4,4,5,5-tetramethyl-1,3,2-dioxaborolan-2-yl)pyridine

Error! Objects cannot be created from editing field codes.

### CAS: 2632985-01-2

### Building block A.14: 2-(difluoromethoxy)-5-methyl-4-(4,4,5,5-tetramethyl-1,3,2-dioxaborolan-2-yl)pyridine

Error! Objects cannot be created from editing field codes.

A mixture of 4-bromo-2-(difluoromethoxy)-5-methyl-pyridine (1805592-30-6) (400 mg, 1.68 mmol, 1.0 equiv.), bis(pinacolato)diboron (1707 mg, 6.72 mmol, 4.0 equiv.), KOAc (330 mg, 3.36 mmol, 2.0 equiv.) and [1,1′-bis(diphenylphosphino)ferrocene]palladium(II) chloride (137 mg, 0.17 mmol, 0.1 equiv.) in 1,4-dioxane (20 mL) was stirred at 80° C. under N₂ atmosphere for 2 hours. The reaction mixture was cooled and filtered. The filtrate was concentrated and the residue was purified by flash chromatography (SiO₂, 10-50% EtOAc in petroleum ether) to give 2-(difluoromethoxy)-5-methyl-4-(4,4,5,5-tetramethyl-1,3,2-dioxaborolan-2-yl)pyridine (433 mg, 1.52 mmol, 90% yield) as a yellow solid. LC-MS: m/z=286.1 [M+H]⁺, ESI pos.

### Building block A.15: 4-methyl-3-(4,4,5,5-tetramethyl-1,3,2-dioxaborolan-2-yl)-1-(2,2,2-trifluoroethyl)pyrazole

Error! Objects cannot be created from editing field codes.

Building block A.15 was synthesized in analogy to Building block A.10 and A.11 using 3-bromo-4-methyl-1H-pyrazole in step 1 to give 4-methyl-3-(4,4,5,5-tetramethyl-1,3,2-dioxaborolan-2-yl)-1-(2,2,2-trifluoroethyl)pyrazole. LC-MS: m/z=209.1 [M+H]⁺, ESI pos.

### Building block A.16: 1-(cyclopropylmethyl)-3-methyl-4-(4,4,5,5-tetramethyl-1,3,2-dioxaborolan-2-yl)pyrazole

Error! Objects cannot be created from editing field codes.

### CAS: 1353003-17-4

### Building block A.17: 1-(difluoromethyl)-4-methyl-3-(4,4,5,5-tetramethyl-1,3,2-dioxaborolan-2-yl)pyrazole

Error! Objects cannot be created from editing field codes.

To a solution of 4-methyl-3-(4,4,5,5-tetramethyl-1,3,2-dioxaborolan-2-yl)-1H-pyrazole (500 mg, 2.4 mmol, 1.0 equiv.) and diethyl(bromodifluoromethyl)phosphonate (1.28 g, 4.81 mmol, 2.0 equiv.) in ACN (10 mL) was added KF (419 mg, 7.21 mmol, 3.0 equiv.). The mixture was stirred at 50° C. for 12 hours. The mixture was diluted with DCM (30 mL) and filtered. The filtrate was washed by saturated aqueous NaHCO₃ (20 mL) and saturated aqueous NH₄Cl (20 mL). The organic layer was dried over Na₂SO₄, filtered and concentrated to give 1-(difluoromethyl)-4-methyl-3-(4,4,5,5-tetramethyl-1,3,2-dioxaborolan-2-yl)pyrazole (120 mg, 0.46 mmol, 17% yield) as brown gum which was used without further purification. ¹H NMR (400 MHz, CDCl₃) δ=7.61 (s, 1H), 7.26 (t, 1H), 2.25 (s, 3H), 1.37 (s, 12H).

### Building block A.18: 3-fluoro-4-(4,4,5,5-tetramethyl-1,3,2-dioxaborolan-2-yl)-1-(2,2,2-trifluoroethyl)pyridin-2-one

Error! Objects cannot be created from editing field codes.

### Step 1: 4-bromo-3-fluoro-1H-pyridin-2-one

A mixture of 4-bromo-2,3-difluoro-pyridine (5.0 g, 25.78 mmol, 1.0 equiv.) in acetic acid (50 mL) and H₂O (25 mL) was stirred at 110° C. for 32 hours. The mixture was concentrated under vacuo and the residue was neutralized with aq. NaHCO₃ (150 mL) and extracted with EtOAc (3×100 mL). The combined organic layers were dried over Na₂SO₄, filtered and concentrated to give 4-bromo-3-fluoro-1H-pyridin-2-one (4.76 g, 24.79 mmol, 94% yield) as white solid which was used without further purification. LC-MS: m/z=191.9 [M+H]⁺, ESI pos.

### Step 2: 4-bromo-3-fluoro-1-(2,2,2-trifluoroethyl)pyridin-2-one

To a solution of 4-bromo-3-fluoro-1H-pyridin-2-one (4.76 g, 24.79 mmol, 1.0 equiv.) in THF (100 mL) was added dropwise LiHMDS (1 M in THF) (49.6 mL, 49.59 mmol, 2.0 equiv.) at 0° C. under N₂ and the reaction mixture was stirred at 0° C. for 1 hour under N₂. 2,2,2-trifluoroethyl trifluoromethanesulfonate (11.51 g, 49.59 mmol, 2.0 equiv.) in THF (50 mL) was added dropwise and the mixture was warmed to RT over 30 minutes. The mixture was heated to 70° C. and stirred for another 16 hours. The mixture was diluted with saturated NH₄Cl (200 mL) and extracted with EtOAc (3×100 mL). The combined organic layers were washed with brine (50 mL), dried over Na₂SO₄, filtered and concentrated. The crude was purified by flash chromatography (SiO₂, 40% EtOAc in petroleum ether) to afford 4-bromo-3-fluoro-1-(2,2,2-trifluoroethyl)pyridin-2-one (5.95 g, 21.71 mmol, 85% yield) as light brown solid. LC-MS: m/z=275.8 [M+H]⁺, ESI pos. ¹H NMR (400 MHz, CDCl₃) δ=7.01 (br d, J=7.5 Hz, 2H), 6.40 (dd, J=5.6, 7.5 Hz, 2H), 4.63 (q, J=8.4 Hz, 4H).

### Step 3: 3-fluoro-4-(4,4,5,5-tetramethyl-1,3,2-dioxaborolan-2-yl)-1-(2,2,2-trifluoroethyl)pyridin-2-one

To a solution of 4-bromo-3-fluoro-1-(2,2,2-trifluoroethyl)pyridin-2-one (2.0 g, 7.3 mmol, 1.0 equiv.) and bis(pinacolato)diboron (3.71 g, 14.6 mmol, 2.0 equiv.) in DMSO (20 mL) was added Pd(dppf)Cl₂ (533 mg, 0.73 mmol, 0.1 equiv.). The reaction mixture was bubbled with N₂ for 10 minutes. KOAc (1.43 g, 14.6 mmol, 2.0 equiv.) was added and the mixture was stirred at 100° C. for 1 hour under N₂. The mixture was cooled and poured into water (100 mL). The brown precipitate was filtered out and the filtrate was extracted with EtOAc (5×50 mL). The combined organic layers were washed with brine (50 mL), dried over Na₂SO₄, filtered and concentrated. The crude was purified by flash chromatography (SiO₂, 50% EtOAc in petroleum ether) to give 3-fluoro-4-(4,4,5,5-tetramethyl-1,3,2-dioxaborolan-2-yl)-1-(2,2,2-trifluoroethyl)pyridin-2-one (1.4 g, 4.36 mmol, 60% yield) as white solid. LC-MS: m/z=240.0 [M+H]⁺, ESI pos. ¹H NMR (400 MHz, CDCl₃) δ=7.04 (d, J=7.0 Hz, 1H), 6.34 (dd, J=4.6, 7.0 Hz, 1H), 4.64 (q, J=8.4 Hz, 2H), 1.36 (s, 12H).

### Building block A.19: 6-(difluoromethoxy)-3-methyl-4-(4,4,5,5-tetramethyl-1,3,2-dioxaborolan-2-yl)pyridazine

Error! Objects cannot be created from editing field codes.

### Step 1: 3,4-dichloro-6-(difluoromethoxy)pyridazine

To a solution of 3,4-dichloro-1H-pyridazin-6-one (9.0 g, 54.55 mmol, 1.0 equiv.) in ACN (100 mL) was added 2,2-difluoro-2-(fluorosulfonyl)acetic acid (19.43 g, 109.1 mmol, 2.0 equiv.). The reaction mixture was stirred at 80° C. for 16 hours to give a white suspension. The reaction mixture was cooled to RT and filtered. The filtrate was concentrated and the residue was purified by flash chromatography (SiO₂, 0-10% EtOAc in petroleum ether) to give 3,4-dichloro-6-(difluoromethoxy)pyridazine (2.7 g, 12.56 mmol, 23% yield) as a light yellow oil. LC-MS: m/z=215.1 [M+H]⁺, ESI pos.

### Step 2: 4-chloro-6-(difluoromethoxy)-3-methyl-pyridazine

To a solution of 3,4-dichloro-6-(difluoromethoxy)pyridazine (1.7 g, 7.91 mmol, 1.0 equiv.) in 1,4-dioxane (20 mL) was added Na₂CO₃ (2.5 g, 23.59 mmol, 2.98 equiv.), trimethylboroxine (2.0 g, 7.97 mmol, 1.01 equiv.) and Pd(dppf)Cl₂DCM (645 mg, 0.79 mmol, 0.1 equiv.) and H₂O (2 mL). The mixture was bubbled with N₂ for 10 minutes. The reaction mixture was stirred at 90° C. under N₂ for 3 hours. The reaction mixture was cooled to RT, poured under stirring into saturated aqueous NH₄Cl (200 ml) and extracted with EtOAc (3×50 mL). The combined organic layers were washed with brine (50 mL), dried over Na₂SO₄, filtered and concentrated. The residue was purified by flash chromatography (SiO₂, 0-10% EtOAc in petroleum ether) to give 4-chloro-6-(difluoromethoxy)-3-methyl-pyridazine (1.3 g, 6.68 mmol, 84% yield) as a white solid. LC-MS: m/z=195.0 [M+H]⁺, ESI pos.

### Step 3: 6-(difluoromethoxy)-3-methyl-4-(4,4,5,5-tetramethyl-1,3,2-dioxaborolan-2-yl)pyridazine

To a solution of 4-chloro-6-(difluoromethoxy)-3-methyl-pyridazine (100 mg, 0.51 mmol, 1.0 equiv.) in 1,4-dioxane (1 mL) was added Pd₂(dba)₃ (47 mg, 0.05 mmol, 0.1 equiv.), bis(pinacolato)diboron (196 mg, 0.77 mmol, 1.5 equiv.), Xphos (49 mg, 0.1 mmol, 0.2 equiv.) and KOAc (152 mg, 1.55 mmol, 3.0 equiv.) at 20° C. The mixture was bubble with N₂ for 10 minutes. The reaction mixture was stirred at 100° C. for 3 hours under N₂. Thiourea on resin (100 mg) was added and the mixture was stirred at RT for 5 minutes. The mixture was filtered and the filtrate was concentrated to give 6-(difluoromethoxy)-3-methyl-4-(4,4,5,5-tetramethyl-1,3,2-dioxaborolan-2-yl)pyridazine (100.0 mg, 0.35 mmol, 68.01% yield) as a brown liquid which was used without further purification. LC-MS: m/z=205.1 [M+H]⁺ (boronic acid), ESI pos.

### Building block A.20: 1-(2,3-difluoropropyl)-3-methyl-4-(4,4,5,5-tetramethyl-1,3,2-dioxaborolan-2-yl)pyrazole

Error! Objects cannot be created from editing field codes.

### Step 1: 4-bromo-3-methyl-1-(oxiran-2-ylmethyl)pyrazole

To a solution of 4-bromo-3-methylpyrazole (5.0 g, 31.06 mmol, 1.0 equiv.) in ACN (100 mL) were added 2-(bromomethyl)oxirane (4.25 g, 31.06 mmol, 1.0 equiv.) and K₂CO₃ (12.88 g, 93.17 mmol, 3.0 equiv.). The mixture was stirred at 50° C. for 16 hours. The mixture was cooled to RT, filtered and the filtrate was concentrated. The crude was purified by flash chromatography (SiO₂, 0-20% EtOAc in petroleum ether) to give 4-bromo-3-methyl-1-(oxiran-2-ylmethyl)pyrazole (4.5 g, 20.73 mmol, 67% yield) as colorless oil. LC-MS: m/z=217.1 [M+H]⁺, ESI pos.

### Step 2: 3-(4-bromo-3-methyl-pyrazol-1-yl)-2-fluoro-propan-1-ol

To a solution of 4-bromo-3-methyl-1-(oxiran-2-ylmethyl)pyrazole (500 mg, 2.3 mmol, 1.0 equiv.) in THF (5 mL) was added tetrabutylammonium fluoride (25.34 mL, 25.34 mmol, 11.0 equiv.) dropwise. The reaction mixture was stirred at 90° C. for 16 hours. The mixture was cooled to RT, poured into water (10 mL) and extracted with EtOAc (3×10 mL). The combined organic layers were washed with brine (20 mL), dried over Na₂SO₄, filtered and concentrated under reduced pressure. The crude was purified by flash chromatography (SiO₂, 0-10% EtOAc in petroleum ether) to give 3-(4-bromo-3-methyl-pyrazol-1-yl)-2-fluoro-propan-1-ol (400 mg, 1.69 mmol, 73% yield) as colorless oil. LC-MS: m/z=237.0 [M+H]⁺, ESI pos.

### Step 3: 4-bromo-1-(2,3-difluoropropyl)-3-methyl-pyrazole

To a solution of 3-(4-bromo-3-methyl-pyrazol-1-yl)-2-fluoro-propan-1-ol (2.6 g, 10.97 mmol, 1.0 equiv.) in toluene (40 mL) was added DAST (3.62 mL, 27.42 mmol, 2.5 equiv.) dropwise at 0° C. under N₂. The mixture was warmed to RT and stirred for 5 minutes. Pyridine (3.0 mL, 37.09 mmol, 3.38 equiv.) was added to the reaction mixture. The reaction mixture was stirred at 70° C. for 16 hours. The mixture was cooled to RT, poured into saturated NaHCO₃ (100 mL) and extracted with EtOAc (3×60 mL). The combined organic layers were washed with brine (50 nL), dried over Na₂SO₄, filtered and concentrated. The crude was purified by preparative HPLC (Phenomenex luna C18 150×40 mm×15 μm, water with FA-ACN) to give 4-bromo-1-(2,3-difluoropropyl)-3-methyl-pyrazole (1.4 g, 5.86 mmol, 53.4% yield)) as brown oil. LC-MS: m/z=239.2 [M+H]⁺, ESI pos. ¹H NMR (400 MHz, CDCl₃) δ=7.57-7.39 (m, 1H), 5.19-4.89 (m, 1H), 4.85-4.34 (m, 4H), 2.39-2.21 (m, 3H).

### Step 4: 1-(2,3-difluoropropyl)-3-methyl-4-(4,4,5,5-tetramethyl-1,3,2-dioxaborolan-2-yl)pyrazole

To a solution of 4-bromo-1-(2,3-difluoropropyl)-3-methyl-pyrazole (1.00 g, 4.18 mmol, 1.0 equiv.) in 1,4-dioxane (40 mL) were added bis(pinacolato)diboron (2.124 g, 8.37 mmol, 2.0 equiv.), 1,1′-bis(diphenylphosphino)ferrocenepalladium(II)dichloride (344 mg, 0.42 mmol, 0.1 equiv.) and KOAc (1.232 g, 12.55 mmol, 3.0 equiv.). The mixture was bubbled with N₂ for 10 minutes. The mixture was stirred at 100° C. for 16 hours. The reaction mixture was cooled to RT, poured into H₂O (50 mL) and extracted with EtOAc (3×40 mL). The combined organic layers were washed with brine (2×20 mL), dried over Na₂SO₄, filtered and concentrated. The crude was purified by preparative HPLC (Phenomenex luna C18 150×40 mm×15 μm, water with FA-ACN) to give 1-(2,3-difluoropropyl)-3-methyl-4-(4,4,5,5-tetramethyl-1,3,2-dioxaborolan-2-yl)pyrazole (270 mg, 0.94 mmol, 14% yield). LC-MS: m/z=287.1 [M+H]⁺, ESI pos.

### Building block B.1: 1-[2-chloro-6-[5-[(6-methylpyridazin-3-yl)amino]benzimidazol-1-yl]-3-pyridyl]ethanone

Error! Objects cannot be created from editing field codes.

Building block B.1 was prepared in Example 76, step 2.

### Building block B.2: 2-chloro-6-[5-[(6-methylpyridazin-3-yl)amino]benzimidazol-1-yl]pyridine-3-carbaldehyde

Error! Objects cannot be created from editing field codes.

### Step 1: 2,6-dichloro-3-(1,3-dioxolan-2-yl)pyridine

In a 250 mL three-necked flask equipped with a Dean-Stark trap, an intensive cooler and a thermometer, 2,6-dichloronicotinaldehyde (3.75 g, 19.2 mmol, 1.0 equiv.) was dissolved in toluene (100 ml). Then, ethane-1,2-diol (1.8 g, 1.6 ml, 28.7 mmol, 1.5 equiv.) and pTsOH (91.1 mg, 479 μmol, 0.025 eq.) were added. The orange solution was heated to reflux overnight with azeotropic removal of H₂O via Dean Stark trap. Once the mixture had cooled down, it was concentrated in vacuum. Residual material was diluted with EtOAc and sat. aq. NaHCO₃. The organic layer was separated and washed with H₂O. The organic layer was dried with Na₂SO₄, filtered and evaporated. The crude was purified by flash column chromatography using 0-50% EtOAc in heptane as eluent. The title compound 2,6-dichloro-3-(1,3-dioxolan-2-yl)pyridine (3.85 g, 91.3%) was obtained as a light yellow oil. LC-MS: m/z=220.1 [M+H]⁺, ESI pos.

### Step 2: 1-[6-chloro-5-(1,3-dioxolan-2-yl)-2-pyridyl]-N-(6-methylpyridazin-3-yl)benzimidazol-5-amine

A mixture of N-(6-methylpyridazin-3-yl)-1H-benzimidazol-5-amine (5.12 g, 22.72 mmol, 1.0 eq.), 2,6-dichloro-3-(1,3-dioxolan-2-yl)pyridine (5.0 g, 22.72 mmol, 1.0 equiv.), K₂CO₃ (6.28 g, 45.44 mmol, 2.0 equiv.) and DMSO (50 mL) was stirred at 60° C. for 24 hours. The reaction mixture was diluted with H₂O and extracted with ethyl acetate. The combined organic layers were dried over anhydrous Na₂SO₄, filtered and concentrated under reduced pressure. The residue was purified by flash column chromatography using 10% MeOH in DCM as eluent to give 1-[6-chloro-5-(1,3-dioxolan-2-yl)-2-pyridyl]-N-(6-methylpyridazin-3-yl)benzimidazol-5-amine (1.0 g, 10.8%) as a brown solid. LC-MS: m/z=409.1 [M+H]⁺, ESI pos.

### Step 4: 2-chloro-6-[5-[(6-methylpyridazin-3-yl)amino]benzimidazol-1-yl]pyridine-3-carbaldehyde

To a solution of 1-[6-chloro-5-(1,3-dioxolan-2-yl)-2-pyridyl]-N-(6-methylpyridazin-3-yl)benzimidazol-5-amine (600 mg, 1.5 mmol, 1.0 equiv.) in HCl/dioxane (8.0 mL, 32.0 mmol, 21.8 eq.) was added HCl (8.0 mL, 48.0 mmol, 32.71 equiv.) at 25° C. and the resulting mixture was stirred at 25° C. for 2 hours. The reaction mixture was filtered and the filter cake was dissolved with H₂O and lyophilized to give 2-chloro-6-[5-[(6-methylpyridazin-3-yl)amino]benzimidazol-1-yl]pyridine-3-carbaldehyde (530.0 mg, 99.0%) as a yellow solid. LC-MS: m/z=365.1 [M+H]⁺, ESI pos.

### Building block B.3: 1-[2-chloro-6-[6-fluoro-5-[(6-methylpyridazin-3-yl)amino]benzimidazol-1-yl]-3-pyridyl]ethanone

Error! Objects cannot be created from editing field codes.

A mixture of 6-fluoro-N-(6-methylpyridazin-3-yl)-1H-benzimidazol-5-amine (Example 93, step 2) (1.70 g, 6.99 mmol, 1.0 equiv.), 1-(2,6-dichloro-3-pyridyl)ethanone (1.33 g, 6.99 mmol, 1.0 equiv.) and DIEA (3.64, L, 20.97 mmol, 3.0 equiv.) in DMSO (15 mL) was stirred at 130° C. for 12 h. The reaction mixture was cooled to RT and water (100 mL) was added. The reaction mixture was filtered and the filtrate was concentrated. The residue was purified by preparative HPLC(Welch Ultimate XB-SiOH 250 mm×70 mm×10 μm; Gradient: 10% to 55% EtOH in hexane) to give product 1-[2-chloro-6-[6-fluoro-5-[(6-methylpyridazin-3-yl)amino]benzimidazol-1-yl]-3-pyridyl]ethanone (1.19 g, 3.01 mmol, 43% yield) as a yellow solid. LC-MS: m/z=397.2 [M+H]⁺, ESI pos. ¹H NMR (400 MHz, DMSO-d6) δ=9.14 (s, 1H), 8.92 (s, 1H), 8.73 (d, J=7.7 Hz, 1H), 8.48 (d, J=8.4 Hz, 1H), 8.23 (d, J=11.6 Hz, 1H), 8.15 (d, J=8.4 Hz, 1H), 7.40-7.34 (m, 1H), 7.28 (d, J=9.0 Hz, 1H), 2.68 (s, 3H), 2.54 (br s, 3H).

| | | | Building | |
|---|---|---|---|---|
| Ex. | Structure | Systematic name | blocks | Analytics |
| 277 | Error! Objects cannot be | 1-[2-[1-(2,2- | A.1 | LC-MS: m/z = 503.0 |
| | created from editing field | difluorocyclopropyl)-3- | and | [M + H]+, ESI pos |
| | codes. | methyl-pyrazol-4-yl]-6- | B.1 | 1H NMR (400 MHz, |
| | | [5-[(6-methylpyridazin-3- | | METHANOL-d4) δ = |
| | | yl)amino]benzimidazol-1- | | 8.83 (s, 1H), 8.28 |
| | | yl]-3-pyridyl]ethanol | | (d, J = 8.4 Hz, 2H), |
| | | | | 8.19 (d, J = 2.0 Hz, |
| | | | | 1H), 8.16 (d, J = 9.0 Hz, |
| | | | | 1H), 7.98 (s, |
| | | | | 1H), 7.84 (d, J = 8.4 Hz, |
| | | | | 1H), 7.62-7.54 |
| | | | | (m, 1H), 7.37 (d, J = |
| | | | | 9.2 Hz, 1H), 7.14 (d, |
| | | | | J = 9.2 Hz, 1H), 5.08- |
| | | | | 5.00 (m, 1H), 4.44- |
| | | | | 4.29 (m, 1H), 2.53 |
| | | | | (s, 3H), 2.39-2.22 |
| | | | | (m, 5H), 1.51-1.42 |
| 278 | Error! Objects cannot be | 1-[2-[1-(2- | A.2 | LC-MS: m/z = 499.2 |
| | created from editing field | methoxypropyl)-3- | and | [M + H]+, ESI pos |
| | codes. | methyl-pyrazol-4-yl]-6- | B.1 | ¹H NMR (400 MHz, |
| | | [5-[(6-methylpyridazin-3- | | METHANOL-d4) δ = |
| | | yl)amino]benzimidazol-1- | | 8.82 (s, 1H), 8.32 |
| | | yl]-3-pyridyl]ethanol | | (br d, J = 4.9 Hz, |
| | | | | 1H), 8.27 (d, J = 8.6 Hz, |
| | | | | 1H), 8.19 (d, J = |
| | | | | 1.8 Hz, 1H), 8.16 (d, |
| | | | | J = 8.9 Hz, 1H), 7.86 |
| | | | | (d, J = 2.2 Hz, 1H), |
| | | | | 7.80 (d, J = 8.4 Hz, |
| | | | | 1H), 7.56 (dd, J = |
| | | | | 2.0, 8.9 Hz, 1H), |
| | | | | 7.36 (d, J = 9.0 Hz, |
| | | | | 1H), 7.14 (d, J = 9.0 Hz, |
| | | | | 1H), 5.10-5.03 |
| | | | | (m, 1H), 4.24-4.09 |
| | | | | (m, 2H), 3.80-3.73 |
| | | | | (m, 1H), 3.33-3.32 |
| | | | | (m, 3H), 2.53 (s, |
| | | | | 3H), 2.35 (s, 3H), |
| | | | | 1.47 (dd, J = 5.1, 6.2 Hz, |
| | | | | 3H), 1.19 (dd, J = |
| | | | | 3.9, 6.2 Hz, 3H) |
| 279 | Error! Objects cannot be | 1-[2-[1-(2- | A.3 | LC-MS: m/z = 499.0 |
| | created from editing field | methoxypropyl)-5- | and | [M + H]+, ESI pos |
| | codes. | methyl-pyrazol-4-yl]-6- | B.1 | ¹H NMR (400 MHz, |
| | | [5-[(6-methylpyridazin-3- | | METHANOL-d4) δ = |
| | | yl)amino]benzimidazol-1- | | 8.83 (s, 1H), 8.30 |
| | | yl]-3-pyridyl]ethanol | | (s, 1H), 8.28 (d, J = |
| | | | | 8.5 Hz, 1H), 8.21- |
| | | | | 8.12 (m, 2H), 7.82 |
| | | | | (d, J = 8.5 Hz, 1H), |
| | | | | 7.71 (d, J = 1.6 Hz, |
| | | | | 1H), 7.56 (dd, J = |
| | | | | 2.0, 8.9 Hz, 1H), |
| | | | | 7.36 (d, J = 9.1 Hz, |
| | | | | 1H), 7.14 (d, J = 9.1 Hz, |
| | | | | 1H), 5.10 (q, J = |
| | | | | 6.5 Hz, 1H), 4.26- |
| | | | | 4.12 (m, 2H), 3.82 |
| | | | | (ddd, J = 4.4, 6.3, |
| | | | | 7.5 Hz, 1H), 3.27 (s, |
| | | | | 3H), 2.53 (s, 3H), |
| | | | | 2.43 (d, J = 1.5 Hz, |
| | | | | 3H), 1.45 (d, J = 6.5 Hz, |
| | | | | 3H), 1.23 (d, J = |
| | | | | 6.3 Hz, 3H) |
| 281 | Error! Objects cannot be | 1-[2-[1-(2,2- | A.4 | LC-MS: m/z = 505.1 |
| | created from editing field | difluoropropyl)-3-methyl- | and | [M + H]+, ESI pos |
| | codes. | pyrazol-4-yl]-6-[5-[(6- | B.1 | ¹H NMR (400 MHz, |
| | | methylpyridazin-3- | | METHANOL-d4) δ = |
| | | yl)amino]benzimidazol-1- | | 8.83 (s, 1H), 8.28 |
| | | yl]-3-pyridyl]ethanol | | (d, J = 8.4 Hz, 1H), |
| | | | | 8.21-8.10 (m, 2H), |
| | | | | 7.94 (s, 1H), 7.83 (d, |
| | | | | J = 8.4 Hz, 1H), 7.58 |
| | | | | (d, J = 9.2 Hz, 1H), |
| | | | | 7.36 (d, J = 9.2 Hz, |
| | | | | 1H), 7.14 (d, J = 9.2 Hz, |
| | | | | 1H), 5.10-5.00 |
| | | | | (m, 1H), 4.60 (s, |
| | | | | 2H), 2.53 (s, 3H), |
| | | | | 2.35 (s, 3H), 1.67 (t, |
| | | | | J = 18.8 Hz, 3H), |
| | | | | 1.47 (d, J = 6.4 Hz, |
| | | | | 3H) |
| 283 | Error! Objects cannot be | 1-[2-(2-ethyl-5-methyl-4- | A.5 | LC-MS: m/z = 466.3 |
| | created from editing field | pyridyl)-6-[5-[(6- | and | [M + H]+, ESI pos |
| | codes. | methylpyridazin-3- | B.1 | ¹H NMR (400 MHz, |
| | | yl)amino]benzimidazol-1- | | METHANOL-d4) δ = |
| | | yl]-3-pyridyl]ethanol | | 8.85 (s, 1H), 8.47 |
| | | | | (s, 1H), 8.36 (d, J = |
| | | | | 8.6 Hz, 1H), 8.28 (br s, |
| | | | | 1H), 8.18 (d, J = |
| | | | | 1.8 Hz, 1H), 8.10 (d, |
| | | | | J = 8.8 Hz, 1H), 7.97 |
| | | | | (d, J = 8.6 Hz, 1H), |
| | | | | 7.52 (dd, J = 2.0, 8.8 Hz, |
| | | | | 1H), 7.35 (d, J = |
| | | | | 9.2 Hz, 1H), 7.29 (br s, |
| | | | | 1H), 7.13 (d, J = |
| | | | | 9.2 Hz, 1H), 4.65 (br s, |
| | | | | 1H), 2.87 (q, J = |
| | | | | 7.7 Hz, 2H), 2.52 (s, |
| | | | | 3H), 2.19 (s, 3H), |
| | | | | 1.38-1.31 (m, 6H) |
| 284 | Error! Objects cannot be | 1-[2-[1-(2,2- | A.6 | LC-MS: m/z = 505.1 |
| | created from editing field | difluoropropyl)-5-methyl- | and | [M + H]+, ESI pos |
| | codes. | pyrazol-4-yl]-6-[5-[(6- | B.1 | ¹H NMR (400 MHz, |
| | | methylpyridazin-3- | | METHANOL-d4) δ = |
| | | yl)amino]benzimidazol-1- | | 8.83 (s, 1H), 8.41 |
| | | yl]-3-pyridyl]ethanol | | (s, 1H), 8.29 (d, J = |
| | | | | 8.8 Hz, 1H), 8.21- |
| | | | | 8.10 (m, 2H), 7.84 |
| | | | | (d, J = 8.8 Hz, 1H), |
| | | | | 7.77 (s, 1H), 7.58 (d, |
| | | | | J = 8.8 Hz, 1H), 7.35 |
| | | | | (d, J = 9.2 Hz, 1H), |
| | | | | 7.13 (d, J = 9.2 Hz, |
| | | | | 1H), 5.13-5.03 (m, |
| | | | | 1H), 4.68-4.62 (m, |
| | | | | 2H), 2.53 (s, 3H), |
| | | | | 2.44 (s, 3H), 1.71 (t, |
| | | | | J = 18.8 Hz, 3H), |
| | | | | 1.46 (d, J = 6.4 Hz, |
| | | | | 3H) |
| 285 | Error! Objects cannot be | 1-[2-(2-methoxy-5- | A.7 | LC-MS: m/z = 468.3 |
| | created from editing field | methyl-4-pyridyl)-6-[5- | and | [M + H]+, ESI pos |
| | codes. | [(6-methylpyridazin-3- | B.1 | ¹H NMR (400 MHz, |
| | | yl)amino]benzimidazol-1- | | METHANOL-d4) δ = |
| | | yl]-3-pyridyl]ethanol | | 8.87-8.84 (m, |
| | | | | 1H), 8.41-8.30 (m, |
| | | | | 2H), 8.24 (s, 1H), |
| | | | | 8.21-8.16 (m, 1H), |
| | | | | 8.16-8.08 (m, 2H), |
| | | | | 8.01-7.86 (m, 1H), |
| | | | | 7.56-7.50 (m, 1H), |
| | | | | 7.35 (d, J = 9.2 Hz, |
| | | | | 1H), 7.13 (d, J = 9.2 Hz, |
| | | | | 1H), 4.97-4.92 |
| | | | | (m, 1H), 3.96 (s, |
| | | | | 3H), 2.52 (s, 3H), |
| | | | | 2.10 (s, 3H), 1.39- |
| | | | | 1.34 (m, 3H) |
| 289 | Error! Objects cannot be | 1-[2-(2-cyclopropyl-5- | A.8 | LC-MS: m/z = 478.3 |
| | created from editing field | methyl-4-pyridyl)-6-[5- | and | [M + H]+, ESI pos |
| | codes. | [(6-methylpyridazin-3- | B.1 | ¹H NMR (400 MHz, |
| | | yl)amino]benzimidazol-1- | | METHANOL-d4) δ = |
| | | yl]-3-pyridyl]ethanol | | 8.85 (s, 1H), 8.40- |
| | | | | 8.34 (m, 2H), 8.18 |
| | | | | (d, J = 1.5 Hz, 1H), |
| | | | | 8.11 (d, J = 8.6 Hz, |
| | | | | 1H), 7.97 (d, J = 8.6 Hz, |
| | | | | 1H), 7.53 (dd, J = |
| | | | | 1.4, 9.0 Hz, 1H), |
| | | | | 7.36 (d, J = 9.2 Hz, |
| | | | | 1H), 7.21-7.11 (m, |
| | | | | 2H), 4.95-4.92 (m, |
| | | | | 1H), 2.53 (s, 3H), |
| | | | | 2.16 (s, 4H), 1.39- |
| | | | | 1.31 (m, 3H), 1.08- |
| | | | | 0.99 (m, 4H) |
| 308 | 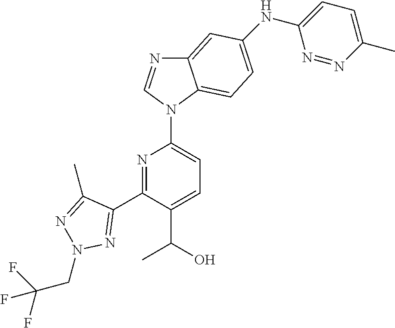 [SMILES: Cc1ccc(Nc2ccc3c(c2)ncn3-c2ccc(C(C)O)c(-c3nn(CC(F)(F)F)nc3C)n2)nn1] | 1-[6-[5-[(6- methylpyridazin-3- yl)amino]benzimidazol-1- yl]-2-[5-methyl-2-(2,2,2- trifluoroethyl)triazol-4- yl]-3-pyridyl]ethanol | A.21 and B.1 | LC-MS: m/z = 510.3 [M + H]+, ESI pos ¹H NMR (400 MHz, METHANOL-d4) δ = 8.85 (s, 1H), 8.46 (br s, 1H), 8.42 (d, J = 8.4 Hz, 1H), 8.22 (d, J = 1.6 Hz, 1H), 8.12 (d, J = 8.8 Hz, 1H), 7.93 (d, J = 8.4 Hz, 1H), 7.61 (dd, J = 2.0, 8.8 Hz, 1H), 7.36 (d, J = 9.2 Hz, 1H), 7.14 (d, J = 9.2 Hz, 1H), 5.45-5.40 (m, 1H), 5.33-5.27 (m, 2H), 2.54 (d, J = 2.9 Hz, 6H), 1.52 (d, J = 6.4 Hz, 3H) |
| 311 | 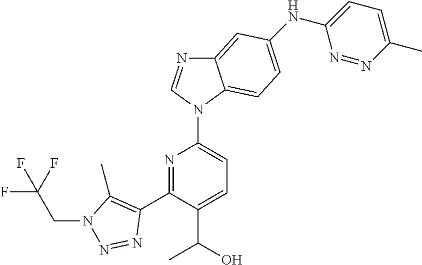 [SMILES: Cc1ccc(Nc2ccc3c(c2)ncn3-c2ccc(C(C)O)c(-c3nnn(CC(F)(F)F)c3C)n2)nn1] | 1-[6-[5-[(6- methylpyridazin-3- yl)amino]benzimidazol-1- yl]-2-[5-methyl-1-(2,2,2- trifluoroethyl)triazol-4- yl]-3-pyridyl]ethanol | A.21 and B.1 | LC-MS: m/z = 510.3 [M + H]+, ESI pos ¹H NMR (400 MHz, METHANOL-d4) δ = 8.87 (s, 1H), 8.42 (d, J = 8.4 Hz, 2H), 8.23 (d, J = 1.6 Hz, 1H), 8.13 (d, J = 8.8 Hz, 1H), 7.94 (d, J = 8.4 Hz, 1H), 7.63 (dd, J = 2.0, 8.8 Hz, 1H), 7.38 (d, J = 9.2 Hz, 1H), 7.15 (d, J = 9.2 Hz, 1H), 5.51- 5.46 (m, 1H), 5.41 (q, J = 8.4 Hz, 2H), |
| | | | | 2.65 (s, 3H), 2.55 (s, |
| | | | | 3H), 1.57 (d, J = 6.4 Hz, |
| | | | | 3H) |
| 313 | Error! Objects cannot be | 1-[2-[1-(2,2- | A.9 | LC-MS: m/z = 491.2 |
| | created from editing field | difluoroethyl)-3-methyl- | and | [M + H]+, ESI pos |
| | codes. | pyrazol-4-yl]-6-[5-[(6- | B.1 | ¹H NMR (400 MHz, |
| | | methylpyridazin-3- | | METHANOL-d4) δ = |
| | | yl)amino]benzimidazol-1- | | 8.90-8.79 (m, |
| | | yl]-3-pyridyl]ethanol | | 1H), 8.28 (d, J = 8.4 Hz, |
| | | | | 1H), 8.23-8.14 |
| | | | | (m, 2H), 7.95 (s, |
| | | | | 1H), 7.83 (d, J = 8.4 Hz, |
| | | | | 1H), 7.57 (d, J = |
| | | | | 8.7 Hz, 1H), 7.36 (d, |
| | | | | J = 9.2 Hz, 1H), 7.14 |
| | | | | (d, J = 9.3 Hz, 1H), |
| | | | | 6.40-6.09 (m, 1H), |
| | | | | 5.08-5.01 (m, 1H), |
| | | | | 4.63-4.57 (m, 2H), |
| | | | | 2.53 (s, 3H), 2.35 (s, |
| | | | | 3H), 1.47 (d, J = 6.5 Hz, |
| | | | | 3H) |
| 315 | Error! Objects cannot be | [6-[5-[(6- | A.10 | LC-MS: m/z = 495.2 |
| | created from editing field | methylpyridazin-3- | and | [M + H]+, ESI pos |
| | codes. | yl)amino]benzimidazol-1- | B.2 | ¹H NMR (400 MHz, |
| | | yl]-2-[3-methyl-1-(2,2,2- | | METHANOL-d4) δ = |
| | | trifluoroethyl)pyrazol-4- | | 8.83 (s, 1H), 8.32- |
| | | yl]-3-pyridyl]methanol | | 8.11 (m, 4H), 7.78 |
| | | | | (d, J = 8.4 Hz, 1H), |
| | | | | 7.60 (dd, J = 2.0, 8.8 Hz, |
| | | | | 1H), 7.35 (d, J = |
| | | | | 9.2 Hz, 1H), 7.12 (d, |
| | | | | J = 9.2 Hz, 1H), 4.97 |
| | | | | (q, J = 8.8 Hz, 2H), |
| | | | | 4.66 (s, 2H), 2.53 (s, |
| | | | | 3H), 2.43 (s, 3H) |
| 316 | Error! Objects cannot be | [6-[5-[(6- | A.11 | LC-MS: m/z = 495.2 |
| | created from editing field | methylpyridazin-3- | and | [M + H]+, ESI pos |
| | codes. | yl)amino]benzimidazol-1- | B.2 | ¹H NMR (400 MHz, |
| | | yl]-2-[5-methyl-1-(2,2,2- | | METHANOL-d4) δ = |
| | | trifluoroethyl)pyrazol-4- | | 8.84 (s, 1H), 8.34- |
| | | yl]-3-pyridyl]methanol | | 8.08 (m, 3H), 7.97 |
| | | | | (s, 1H), 7.81 (d, J = |
| | | | | 8.4 Hz, 1H), 7.60 |
| | | | | (dd, J = 2.0, 8.8 Hz, |
| | | | | 1H), 7.35 (d, J = 9.2 Hz, |
| | | | | 1H), 7.13 (d, J = |
| | | | | 9.2 Hz, 1H), 5.02 (q, |
| | | | | J = 8.8 Hz, 2H), 4.69 |
| | | | | (s, 2H), 2.52 (d, J = |
| | | | | 4.4 Hz, 6H) |
| 332 | Error! Objects cannot be | 1-[6-[5-[(6- | A.12 | LC-MS: m/z = 506.4 |
| | created from editing field | methylpyridazin-3- | and | [M + H]+, ESI pos |
| | codes. | yl)amino]benzimidazol-1- | B.1 | ¹H NMR (400 MHz, |
| | | yl]-2-[5-methyl-2- | | METHANOL-d4) δ = |
| | | (trifluoromethyl)-4- | | 8.86 (s, 1H), 8.77 |
| | | pyridyl]-3- | | (s, 1H), 8.49-8.42 |
| | | pyridyl]ethanol | | (m, 1H), 8.38 (d, J = |
| | | | | 8.3 Hz, 1H), 8.19 (d, |
| | | | | J = 1.8 Hz, 1H), 8.08 |
| | | | | (d, J = 8.8 Hz, 1H), |
| | | | | 8.02 (d, J = 8.6 Hz, |
| | | | | 1H), 7.83 (br s, 1H), |
| | | | | 7.54 (d, J = 9.0 Hz, |
| | | | | 1H), 7.34 (d, J = 9.2 Hz, |
| | | | | 1H), 7.11 (d, J = |
| | | | | 9.2 Hz, 1H), 4.92- |
| | | | | 4.90 (m, 1H), 2.52 |
| | | | | (s, 3H), 2.32 (s, 3H), |
| | | | | 1.37 (br d, J = 6.4 Hz, |
| | | | | 3H) |
| 335 | Error! Objects cannot be | 1-[2-[2-(difluoromethyl)- | A.13 | LC-MS: m/z = 488.4 |
| | created from editing field | 5-methyl-4-pyridyl]-6-[5- | and | [M + H]+, ESI pos |
| | codes. | [(6-methlpyridazin-3- | B.1 | ¹H NMR (400 MHz, |
| | | yl)amino]benzimidazol-1- | | METHANOL-d4) δ = |
| | | yl]-3-pyridyl]ethanol | | 8.87 (s, 1H), 8.70 |
| | | | | (s, 1H), 8.38 (d, J = |
| | | | | 8.5 Hz, 1H), 8.32 (br s, |
| | | | | 1H), 8.19 (d, J = |
| | | | | 2.0 Hz, 1H), 8.10 (d, |
| | | | | J = 9.0 Hz, 1H), 8.02 |
| | | | | (d, J = 8.5 Hz, 1H), |
| | | | | 7.67 (br d, J = 6.9 Hz, |
| | | | | 1H), 7.53 (dd, J = |
| | | | | 2.1, 8.8 Hz, 1H), |
| | | | | 7.35 (d, J = 9.1 Hz, |
| | | | | 1H), 7.12 (d, J = 9.1 Hz, |
| | | | | 1H), 6.99-6.63 |
| | | | | (m, 1H), 4.91 (br s, |
| | | | | 1H), 2.52 (s, 3H), |
| | | | | 2.29 (s, 3H), 1.41- |
| | | | | 1.32 (m, 3H) |
| 346 | Error! Objects cannot be | 1-[2-[2- | A.14 | LC-MS: m/z = 522.4 |
| | created from editing field | (difluoromethoxy)-5- | and | [M + H]+, ESI pos |
| | codes. | methyl-4-pyridyl]-6-[6- | B.3 | ¹H NMR (400 MHz, |
| | | fluoro-5-[(6- | | METHANOL-d4) δ = |
| | | methylpyridazin-3- | | 8.91 (s, 1H), 8.49 |
| | | yl)amino]benzimidazol-1- | | (d, J = 7.5 Hz, 1H), |
| | | yl]-3-pyridyl]ethanol | | 8.36 (d, J = 8.6 Hz, |
| | | | | 1H), 8.26 (s, 1H), |
| | | | | 8.04-7.98 (m, 2H), |
| | | | | 7.81-7.41 (m, 1H), |
| | | | | 7.38 (d, J = 9.1 Hz, |
| | | | | 1H), 7.19 (d, J = 9.1 Hz, |
| | | | | 1H), 7.00 (br s, |
| | | | | 1H), 4.97-4.90 (m, |
| | | | | 1H), 2.53 (s, 3H), |
| | | | | 2.17 (s, 3H), 1.37 (br s, |
| | | | | 3H) |
| 349 | Error! Objects cannot be | 1-[6-[5-[(6- | A.15 | LC-MS: m/z = 508.9 |
| | created from editing field | methylpyridazin-3- | and | [M + H]+, ESI pos |
| | codes. | yl)amino]benzimidazol-1- | B.1 | ¹H NMR (400 MHz, |
| | | yl]-2-[4-methyl-1-(2,2,2- | | METHANOL-d4) δ = |
| | | trifluoroethyl)pyrazol-3- | | 8.85 (s, 1H), 8.34 |
| | | yl]-3-pyridyl]ethanol | | (d, J = 8.4 Hz, 1H), |
| | | | | 8.23-8.14 (m, 2H), |
| | | | | 7.88 (d, J = 8.6 Hz, |
| | | | | 1H), 7.72 (s, 1H), |
| | | | | 7.60 (dd, J = 2.1, 8.9 Hz, |
| | | | | 1H), 7.37 (d, J = |
| | | | | 9.2 Hz, 1H), 7.14 (d, |
| | | | | J = 9.2 Hz, 1H), 5.29 |
| | | | | (q, J = 6.4 Hz, 1H), |
| | | | | 4.99 (q, J = 8.5 Hz, |
| | | | | 2H), 2.54 (s, 3H), |
| | | | | 2.25 (s, 3H), 1.49 (d, |
| | | | | J = 6.4 Hz, 3H) |
| 356 | Error! Objects cannot be | 1-[2-[1- | A.16 | LC-MS: m/z = 499.4 |
| | created from editing field | (cyclopropylmethyl)-3- | and | [M + H]+, ESI pos |
| | codes. | methyl-pyrazol-4-yl]-6- | B.3 | ¹H NMR (400 MHz, |
| | | [6-fluoro-5-[(6- | | METHANOL-d4) δ = |
| | | methylpyridazin-3- | | 8.88 (s, 1H), 8.49 |
| | | yl)amino]benzimidazol-1- | | (d, J = 7.5 Hz, 1H), |
| | | yl]-3-pyridyl]ethanol | | 8.43 (s, 1H), 8.27 (d, |
| | | | | J = 8.4 Hz, 1H), 8.14 |
| | | | | (d, J = 11.4 Hz, 1H), |
| | | | | 7.94 (s, 1H), 7.83 (d, |
| | | | | J = 8.5 Hz, 1H), 7.38 |
| | | | | (d, J = 9.1 Hz, 1H), |
| | | | | 7.20 (d, J = 9.1 Hz, |
| | | | | 1H), 5.06 (q, J = 6.3 Hz, |
| | | | | 1H), 4.05 (d, J = |
| | | | | 3H), 2.36 (s, 3H), |
| | | | | 1.48 (d, J = 6.4 Hz, |
| | | | | 3H), 1.41-1.35 (m, |
| | | | | 1H), 0.69-0.64 (m, |
| | | | | 2H), 0.48-0.43 (m, |
| | | | | 2H) |
| 363 | Error! Objects cannot be | 1-[2-[1-(difluoromethyl)- | A.17 | LC-MS: m/z = 477.2 |
| | created from editing field | 4-methyl-pyrazol-3-yl]-6- | and | [M + H]+, ESI pos |
| | codes. | [5-[(6-methylpyridazin-3- | B.1 | ¹H NMR (400 MHz, |
| | | yl)amino]benzimidazol-1- | | CHLOROFORM-d) |
| | | yl]-3-pyridyl]ethanol | | δ = 8.64 (s, 1H), |
| | | | | 8.37 (s, 1H), 8.22 (d, |
| | | | | J = 8.5 Hz, 1H), 8.02 |
| | | | | (d, J = 8.8 Hz, 1H), |
| | | | | 7.80 (s, 1H), 7.77 (d, |
| | | | | J = 1.8 Hz, 1H), 7.63 |
| | | | | (d, J = 8.4 Hz, 1H), |
| | | | | 7.37 (s, 1H), 7.35 |
| | | | | (dd, J = 2.0, 8.8 Hz, |
| | | | | 1H), 7.22 (s, 1H), |
| | | | | 7.18-7.11 (m, 2H), |
| | | | | 7.07 (s, 1H), 5.11 (q, |
| | | | | J = 6.5 Hz, 1H), 2.60 |
| | | | | (s, 3H), 2.36 (s, 3H), |
| | | | | 1.63 (d, J = 6.6 Hz, |
| | | | | 3H) |
| 386 | Error! Objects cannot be | 3-fluoro-4-[3-(1- | A.18 | LC-MS: m/z = 540.1 |
| | created from editing field | hydroxyethyl)-6-[5-[(6- | and | [M + H]+, ESI pos |
| | codes. | methylpyridazin-3- | B.1 | |
| | | yl)amino]benzimidazol-1- | | |
| | | yl]-2-pyridyl]-1-(2,2,2- | | |
| | | trifluoroethyl)pyridin-2- | | |
| | | one | | |
| 396 | Error! Objects cannot be | 1-[2-[6- | A.19 | LC-MS: m/z = 505.3 |
| | created from editing field | (difluoromethoxy)-3- | and | [M + H]+, ESI pos |
| | codes. | methyl-pyridazin-4-yl]-6- | B.1 | ¹H NMR (400 MHz, |
| | | [5-[(6-methylpyridazin-3- | | DMSO-d6) δ = 9.18 |
| | | yl)amino]benzimidazol-1- | | (s, 1H), 8.98 (s, 1H), |
| | | yl]-3-pyridyl]ethanol | | 8.43-8.40 (m, 1H), |
| | | | | 8.31 (d, J = 8.6 Hz, |
| | | | | 1H), 8.28 (s, 1H), |
| | | | | 8.15-8.11 (m, 1H), |
| | | | | 8.03 (d, J = 8.9 Hz, |
| | | | | 1H), 7.95 (s, 1H), |
| | | | | 7.77 (s, 1H), 7.65 (s, |
| | | | | 1H), 7.45-7.40 (m, |
| | | | | 1H), 7.32 (d, J = 9.1 Hz, |
| | | | | 1H), 7.07 (d, J = |
| | | | | 9.0 Hz, 1H), 2.47 (s, |
| | | | | 3H), 2.46 (s, 3H), |
| | | | | 1.29 (br d, J = 5.6 Hz, |
| | | | | 3H) |
| 399 | Error! Objects cannot be | 1-[2-[1-(2,3- | A.20 | LC-MS: m/z = 505.1 |
| | created from editing field | difluoropropyl)-3-methyl- | and | [M + H]+, ESI pos |
| | codes. | pyrazol-4-yl]-6-[5-[(6- | B.1 | ¹H NMR (400 MHz, |
| | | methylpyridazin-3- | | METHANOL-d4) δ = |
| | | yl)amino]benzimidazol-1- | | 8.85 (s, 1H), 8.30 |
| | | yl]-3-pyridyl]ethanol | | (d, J = 8.4 Hz, 1H), |
| | | | | 8.25-8.20 (m, 1H), |
| | | | | 8.20-8.16 (m, 1H), |
| | | | | 7.94 (s, 1H), 7.87- |
| | | | | 7.80 (m, 1H), 7.62- |
| | | | | 7.55 (m, 1H), 7.39 |
| | | | | (d, J = 9.1 Hz, 1H), |
| | | | | 7.16 (d, J = 9.1 Hz, |
| | | | | 1H), 5.09-5.05 (m, |
| | | | | 1H), 4.80-4.56 (m, |
| | | | | 5H), 2.55 (s, 3H), |
| | | | | 2.37 (s, 3H), 1.49 (d, |
| | | | | J = 6.5 Hz, 3H) |

### Examples 275, 282, 293, 294, 297, 302, 307, 314, 317, 319, 333, 337, 345, 347, 348, 353-355, 357, 361, 362, 364, 367, 371-379, 381-385, 388-391, 394, 400, 402, 403

In analogy to Example 65, Examples in the following table were prepared, using the respective building blocks C.X (C.1 to C.7) and D.X (D.1 to D.8) in step 1 and E.X (E.1 to E.19) in step 2.

### Building block C.1: 6-Bromo-5-(trifluoromethyl)-1H-benzimidazole

Error! Objects cannot be created from editing field codes.

### CAS: 1008361-62-3

### Building block C.2: 5-bromo-6-(oxetan-3-yloxy)-1˜{H}-benzimidazole

Error! Objects cannot be created from editing field codes.

### Step 1: 4-bromo-2-nitro-5-(oxetan-3-yloxy)aniline

To a solution of oxetan-3-ol (4.26 g, 57.46 mmol, 1.5 eq.) in THF (90 mL) under N₂ cooled to 0° C. was added sodium hydride (60% in oil) (2.3 g, 57.61 mmol, 1.5 eq.) in portions and the mixture was stirred at 0° C. for 30 minutes. 4-bromo-5-fluoro-2-nitro-aniline (9.0 g, 38.3 mmol, 1.0 eq.) was added and the reaction mixture was allowed to warm to RT and was stirred at RT for 12 hours. The reaction was quenched with H₂O (100 mL) which formed a precipitate. The mixture was filtered and the filter cake was dried under reduced pressure to give 4-bromo-2-nitro-5-(oxetan-3-yloxy)aniline (4.0 g, 13.84 mmol, 36% yield). The filtrate was extracted with EtOAc (3×250 mL). The combined organic layers were dried over Na₂SO₄, filtered and concentrated. The residue was triturated in a mixture of petrol ether/EtOAc (10:1, 30 mL) at RT for 30 minutes. The mixture was filtered and the filter cake was dried under reduced pressure to give 4-bromo-2-nitro-5-(oxetan-3-yloxy)aniline (7.0 g, 24.21 mmol, 63% yield) as yellow solid. ESI pos [M+H]⁺ 289.0. ¹H NMR (400 MHz, DMSO-d₆) δ=8.16 (s, 1H), 7.56 (s, 2H), 6.21 (s, 1H), 5.31 (q, J=5.4 Hz, 1H), 4.93 (t, J=6.8 Hz, 2H), 4.59 (dd, J=5.2, 7.6 Hz, 2H).

### Step 2: 4-bromo-5-(oxetan-3-yloxy)benzene-1,2-diamine

To a solution of 4-bromo-2-nitro-5-(oxetan-3-yloxy)aniline (10.5 g, 36.32 mmol, 1.0 eq.) in EtOH (120 mL) under N₂ was added Fe (10.14 g, 181.61 mmol, 5.0 eq.), NH₄Cl (19.43 g, 363.22 mmol, 10.0 eq.) and H₂O (40 mL). The reaction mixture was stirred under N₂ at 50° C. for 12 hours. The reaction mixture was cooled to RT and filtered. The filtrate was diluted with H₂O (100 mL) and extracted with EtOAc (3×100 mL). The combined organic layers were concentrated to give 4-bromo-5-(oxetan-3-yloxy)benzene-1,2-diamine (9.4 g, 36.28 mmol, quant. yield) as black solid. ESI pos [M+H]⁺ 259.0.

### Step 3: 5-bromo-6-(oxetan-3-yloxy)-1H-benzimidazole, formic acid

A solution of 4-bromo-5-(oxetan-3-yloxy)benzene-1,2-diamine (9.0 g, 34.74 mmol, 1.0 eq.) and formic acid (8.0 mL, 41463.19 mmol, 1193.68 eq.) in triethyl orthoformate (80 mL) was stirred at 80° C. for 12 hours. The reaction mixture was cooled to RT and concentrated. The residue was purified by preparative HPLC (Phenomenex luna C18 10 μm, 150 mm×40 mm, water+0.1% FA−ACN) to give 5-bromo-6-(oxetan-3-yloxy)-1H-benzimidazole (8.6 g, 31.96 mmol, 91% yield) as a white solid. ESI pos [M+H]⁺ 270.9. ¹H NMR (400 MHz, CDCl₃) δ=8.16 (s, 1H), 8.05 (s, 1H), 7.88 (s, 1H), 6.80 (s, 1H), 5.29 (td, J=5.6, 11.2 Hz, 1H), 5.04 (t, J=6.8 Hz, 2H), 4.95-4.84 (m, 2H).

### Building block C.3: 6-Bromo-1H-benzimidazole

Error! Objects cannot be created from editing field codes.

### CAS: 4887-88-1

### Building block C.4: 4-[2-[(6-bromo-3˜{H}-benzimidazol-5-yl)oxy]ethyl]morpholine

### Step 3: 4-bromo-5-(2-morpholinoethoxy)-2-nitro-aniline

To a solution of 2-morpholinoethanol (614 mg, 4.68 mmol, 1.1 eq) in THF (50 mL) was added NaH (204 mg, 5.1 mmol, 1.2 eq) at 0° C. under N₂. The reaction mixture was stirred at 0° C. for 30 min. 4-bromo-5-fluoro-2-nitro-aniline (1.00 g, 4.26 mmol, 1.0 eq) was added at 0° C. under N₂. The reaction mixture was stirred at 20° C. for 14 h. The reaction mixture was poured into sat. aq. NH₄Cl (300 mL) under vigorous stirring and extracted with EtOAc (3×200 mL). The combined organic layers were washed with brine (50 mL), dried over Na₂SO₄, filtered and concentrated. The residue was purified by flash chromatography (SiO₂, 0-10% MeOH in DCM) to give 4-bromo-5-(2-morpholinoethoxy)-2-nitro-aniline (800 mg, 2.31 mmol, 54% yield) as yellow solid. LC-MS: m/z=348.1 [M+H]⁺, ESI pos. ¹H NMR (400 MHz, DMSO-d6) δ=8.12 (s, 1H), 7.57 (s, 2H), 6.60 (s, 1H), 4.15 (t, J=5.6 Hz, 2H), 3.59-3.53 (m, 4H), 2.75 (t, J=5.6 Hz, 2H), 2.53-2.51 (m, 4H).

### Step 3: 4-bromo-5-(2-morpholinoethoxy)benzene-1,2-diamine

To a solution of 4-bromo-5-(2-morpholinoethoxy)-2-nitro-aniline (650 mg, 1.88 mmol, 1.0 eq) in EtOH (10 mL) was added Fe (524 mg, 9.39 mmol, 5.0 eq), NH₄Cl (1.00 g, 18.78 mmol, 10 eq) and H₂O (3 mL). The reaction mixture was stirred under N₂ at 50° C. for 12 h. The reaction mixture was diluted with EtOAc (50 mL). The mixture was filtered and the filtrate was added into H₂O (20 mL). The aqueous phase was extracted with EtOAc (3×30 mL). Combined organic layers were washed with brine (30 mL), dried over Na₂SO₄, filtered and concentrated to give 4-bromo-5-(2-morpholinoethoxy)benzene-1,2-diamine (600 mg, 1.9 mmol, 71% yield) as brown oil which was used without further purification. LC-MS: m/z=316.0 [M+H]⁺, ESI pos.

### Step 3: 4-[2-[(6-bromo-1H-benzimidazol-5-yl)oxy]ethyl]morpholine

To a solution of 4-bromo-5-(2-morpholinoethoxy)benzene-1,2-diamine (500 mg, 1.58 mmol, 1.0 eq) in EtOH (10 mL) were added trimethyl orthoformate (1.73 mL, 15.81 mmol, 10 eq) and TsOH (24 mg, 0.16 mmol, 0.1 eq). The mixture was stirred at 80° C. for 2 h. The reaction mixture was cooled to RT and diluted with sat. aq. NaHCO₃ (50 ml) and extracted with EtOAc (3×30 mL). The combined organic layers were washed with brine (20 mL), dried over Na₂SO₄, filtered and concentrated to give 4-[2-[(6-bromo-1H-benzimidazol-5-yl)oxy]ethyl]morpholine (500 mg, 1.53 mmol, 97% yield) as yellow solid which was used without further purification. LC-MS: m/z=328.0 [M+H]⁺, ESI pos.

### Building block C.5: 6-Bromo-5-fluoro-1H-benzimidazole

Error! Objects cannot be created from editing field codes.

### CAS: 1008360-84-6

### Building block C.6: 6-Bromo-5-(difluoromethoxy)-1H-benzimidazole

Error! Objects cannot be created from editing field codes.

### CAS: 1805750-52-0

### Building block C.7: 6-Bromo-5-methoxy-1H-benzimidazole

Error! Objects cannot be created from editing field codes.

### CAS:1008361-65-6

### Building block D.1: 1-(3-acetyl-6-chloro-2-pyridyl)-5-methyl-pyrazole-3-carbonitrile

Error! Objects cannot be created from editing field codes.

Building block D.1 was prepared in Example 64, step 1.

### Building block D.2: 1-[6-chloro-2-[3-methyl-1-(2,2,2-trifluoroethyl)pyrazol-4-yl]-3-pyridyl]ethanone and 1-[6-chloro-2-[5-methyl-1-(2,2,2-trifluoroethyl)pyrazol-4-yl]-3-pyridyl]ethanone

Error! Objects cannot be created from editing field codes. Error! Objects cannot be created from editing field codes.

A mixture of 3-methyl-4-(4,4,5,5-tetramethyl-1,3,2-dioxaborolan-2-yl)-1-(2,2,2-trifluoroethyl)pyrazole (Building block A.10) (280 mg, 0.97 mmol, 1.0 eq), 1-(2-bromo-6-chloro-3-pyridyl)ethanone (1256789-68-O) (226 mg, 0.97 mmol, 1.0 eq), K₂CO₃ (267 mg, 1.93 mmol, 2.0 eq) and Pd(dppf)Cl₂CH₂Cl₂ (79 mg, 0.1 mmol, 0.1 eq) in 1,4-dioxane (4 mL) and H₂O (0.400 mL) was stirred at 80° C. under N₂ atmosphere for 12 hours. The reaction mixture was cooled to RT and concentrated. The residue was purified by flash chromatography (SiO₂, 0-20% EtOAc in PE) to give a mixture of 1-[6-chloro-2-[5-methyl-1-(2,2,2-trifluoroethyl)pyrazol-4-yl]-3-pyridyl]ethanone and 1-[6-chloro-2-[3-methyl-1-(2,2,2-trifluoroethyl)pyrazol-4-yl]-3-pyridyl]ethanone (252 mg, 0.79 mmol, 82% yield) (mixture) as yellow oil. LC-MS: m/z=318.0 [M+H]⁺, ESI pos.

### Building block D.3: 1-[6-chloro-2-[3-(difluoromethyl)-5-methyl-pyrazol-1-yl]-3-pyridyl]ethanone and 1-[6-chloro-2-[5-(difluoromethyl)-3-methyl-pyrazol-1-yl]-3-pyridyl]ethanone

Error! Objects cannot be created from editing field codes.

Building block D.3 was prepared in Example 128, step 1.

### Building block D.4: 1-[6-chloro-2-[3-(difluoromethoxy)-5-methyl-pyrazol-1-yl]-3-pyridyl]ethanone

Error! Objects cannot be created from editing field codes.

To a colorless solution of 3-(difluoromethoxy)-5-methyl-1H-pyrazole (2108002-48-6) (2.8 g, 18.9 mmol, 1.0 eq) in DMSO (30 mL) was added K₂CO₃ (7.84 g, 56.71 mmol, 3.0 eq) and 1-(6-chloro-2-fluoro-3-pyridyl)ethanone (3.35 g, 18.9 mmol, 1.0 eq). The mixture was stirred at 30° C. for 5 hours. The reaction mixture was poured into H₂O (100 mL) and extracted with EtOAc (3×50 mL). The combined organic layers were washed with brine (20 mL), dried over Na₂SO₄, filtered and concentrated. The residue was purified by flash chromatography (SiO₂, 10-30% EtOAc in PE) to give 1-[6-chloro-2-[3-(difluoromethoxy)-5-methyl-pyrazol-1-yl]-3-pyridyl]ethanone (1.5 g, 4.97 mmol, 24% yield) as a white solid. LC-MS: m/z=302.0 [M+H]⁺, ESI pos. ¹H NMR (400 MHz, CDCl₃) δ=7.81 (d, J=7.9 Hz, 1H), 7.32 (d, J=7.9 Hz, 1H), 6.83 (t, J=72.9 Hz, 1H), 5.92 (s, 1H), 2.61 (s, 3H), 2.17 (s, 3H).

### Building block D.5: 1-[6-chloro-2-(3-chloro-5-methyl-pyrazol-1-yl)-3-pyridyl]ethanone

Error! Objects cannot be created from editing field codes.

Building block D.5 was prepared in analogy to Building block D.4 using 1-(6-chloro-2-fluoro-3-pyridyl)ethanone (300 mg, 1.73 mmol, 1.0 eq) and 3-chloro-5-methyl-1H-pyrazole (201 mg, 1.73 mmol, 1.0 eq) to give 1-[6-chloro-2-(3-chloro-5-methyl-pyrazol-1-yl)-3-pyridyl]ethanone (330 mg, 1.22 mmol, 71% yield) as colorless oil. LC-MS: m/z=269.8 [M+H]⁺, ESI pos. ¹H NMR (400 MHz, CDCl₃) δ=7.91 (d, J=7.9 Hz, 1H), 7.40 (d, J=8.1 Hz, 1H), 6.22 (s, 1H), 2.61 (s, 3H), 2.17 (s, 3H).

### Building block D.6: 1-(6-chloro-3-formyl-2-pyridyl)-5-methyl-pyrazole-3-carbonitrile

Error! Objects cannot be created from editing field codes.

Building block D.6 was prepared in analogy to Building block DA using 6-chloro-2-fluoro-pyridine-3-carbaldehyde (1.0 g, 6.27 mmol, 1.0 eq) and 5-methyl-1H-pyrazole-3-carbonitrile (738 mg, 6.89 mmol, 1.1 eq) to give 1-(6-chloro-3-formyl-2-pyridyl)-5-methyl-pyrazole-3-carbonitrile (800 mg, 3.24 mmol, 52% yield) as colorless oil. LC-MS: m/z=247.1 [M+H]⁺, ESI pos. ¹H NMR (400 MHz, DMSO-d6) δ ppm 9.80 (s, 1H) 8.43 (d, J=8.13 Hz, 1H) 7.92 (d, J=8.25 Hz, 1H) 7.15 (d, J=0.75 Hz, 1H) 2.54 (d, J=0.63 Hz, 3H).

### Building block D.7: methyl 6-chloro-2-[3-(difluoromethyl)-5-methyl-pyrazol-1-yl]pyridine-3-carboxylate

Error! Objects cannot be created from editing field codes.

Building block D.7 was prepared in analogy to Building block DA using 6-chloro-2-fluoro-nicotinic acid methyl ester (1 g, 5.28 mmol, 1.0 eq) and 3-(difluoromethyl)-5-methyl-1H-pyrazole (836.27 mg, 6.33 mmol, 1.2 eq) to give 6-chloro-2-[3-(difluoromethyl)-5-methyl-pyrazol-1-yl]nicotinic acid methyl ester (943 mg, 3.12 mmol, 59% yield) as colorless oil. LC-MS: m/z=302.05 [M+H]⁺, ESI pos. ¹H NMR (600 MHz, CDCl₃) δ ppm 8.11 (d, J=8.2 Hz, 1H), 7.44 (d, J=8.1 Hz, 1H), 6.61 (t, J=55.0 Hz, 1H), 6.43 (d, J=0.7 Hz, 1H), 3.72 (s, 3H), 2.54 (d, J=0.7 Hz, 3H).

### Building block D.8: methyl 2-[3,5-bis(difluoromethyl)pyrazol-1-yl]-6-chloro-pyridine-3-carboxylate

Error! Objects cannot be created from editing field codes.

Building block D.8 was prepared in analogy to Building block DA using 6-chloro-2-fluoro-nicotinic acid methyl ester (250 mg, 1.32 mmol, 1.0 eq) and 3,5-bis(difluoromethyl)-1H-pyrazole (243.84 mg, 1.45 mmol, 1.1 eq) to give 2-[3,5-bis(difluoromethyl)pyrazol-1-yl]-6-chloro-nicotinic acid methyl ester (392 mg, 1.16 mmol, 88% yield) as colorless oil. LC-MS: m/z=338.0 [M+H]⁺, ESI pos.

### Building block E.1: 3-amino-6-methylpyridazine

Error! Objects cannot be created from editing field codes.

### CAS: 18591-82-7

### Building block E.2: 6-[2-[2-[2-[2-[2-[2-(2-methoxyethoxy)ethoxy]ethoxy]ethoxy]ethoxy]ethoxy]ethoxy]pyridazin-3-amine

Error! Objects cannot be created from editing field codes.

### Step 1: 3-chloro-6-[2-[2-[2-[2-[2-[2-(2-methoxyethoxy)ethoxy]ethoxy]ethoxy]ethoxy]ethoxy]ethoxy]pyridazine

To a solution of 2,5,8,11,14,17,20-heptaoxadocosan-22-ol (3.0 g, 8.81 mmol, 1.0 eq) in DMF (30 mL) was added NaH (0.6 g, 17.63 mmol, 2.0 eq) at 0° C. The reaction was stirred at 0° C. for 30 minutes. 3,6-dichloropyridazine (1.44 g, 9.69 mmol, 1.1 eq) was added and the reaction mixture was stirred at 25° C. for 16 hours. The reaction mixture was diluted with saturated aqueous NH₄Cl (50 mL) and extracted with EtOAc (3×50 mL), The combined organic layers were washed with brine (20 mL), dried over Na₂SO₄, filtered and concentrated. The residue was purified by flash chromatography (SiO₂, 10-30% EtOAc in PE) to give 3-chloro-6-[2-[2-[2-[2-[2-[2-(2-methoxyethoxy)ethoxy]ethoxy]ethoxy]ethoxy]ethoxy]ethoxy]pyridazine (2.2 g, 4.86 mmol, 55% yield) as yellow oil. LC-MS: m/z=453.1 [M+H]⁺, ESI pos. ¹H NMR (400 MHz, CDCl₃) δ ppm 7.39 (d, J=9.17 Hz, 1H) 7.03 (d, J=9.17 Hz, 1H) 4.61-4.75 (m, 2H) 3.87-3.94 (m, 2H) 3.63-3.73 (m, 22H) 3.53-3.59 (m, 2H) 3.35-3.41 (m, 3H).

### Step 2: tert-butyl N-[6-[2-[2-[2-[2-[2-[2-(2-methoxyethoxy)ethoxy]ethoxy]ethoxy]ethoxy]ethoxy]ethoxy]pyridazin-3-yl]carbamate

A mixture of 3-chloro-6-[2-[2-[2-[2-[2-[2-(2-methoxyethoxy)ethoxy]ethoxy]ethoxy]ethoxy]ethoxy]ethoxy]pyridazine (1.0 g, 2.21 mmol, 1.0 eq), tert-butyl carbamate (517 mg, 4.42 mmol, 2.0 eq), Cs₂CO₃ (2.16 g, 6.62 mmol, 3.0 eq), 9,9-dimethyl-4,5-bis(diphenylphosphino)xanthene (256 mg, 0.44 mmol, 0.2 eq) and tris(dibenzylideneacetone)dipalladium (0) (202 mg, 0.22 mmol, 0.1 eq) in 1,4-dioxane (10 mL) was stirred at 80° C. for 3 h under N₂ atmosphere. The mixture was cooled to RT, diluted with H₂O (50 mL) and extracted with EtOAc (3×50 mL). The combined organic layers were washed with brine (20 mL), dried over Na₂SO₄, filtered and concentrated. The residue was purified by flash chromatography (SiO₂, 100% EtOAc) to give tert-butyl N-[6-[2-[2-[2-[2-[2-[2-(2-methoxyethoxy)ethoxy]ethoxy]ethoxy]ethoxy]ethoxy]ethoxy]pyridazin-3-yl]carbamate 00800 mg, 1.50 mmol, 68% yield). LC-MS: m/z=534.2 [M+H]⁺, ESI pos.

### Step 3: 6-[2-[2-[2-[2-[2-[2-(2-methoxyethoxy)ethoxy]ethoxy]ethoxy]ethoxy]ethoxy]ethoxy]pyridazin-3-amine

A mixture of tert-butyl N-[6-[2-[2-[2-[2-[2-[2-(2-methoxyethoxy)ethoxy]ethoxy]ethoxy]ethoxy]ethoxy]ethoxy]pyridazin-3-yl]carbamate (700 mg, 1.31 mmol, 1.0 eq) in TFA (2.0 mL) and DCM (6 mL) was stirred at 25° C. for 16 hours. The reaction mixture was concentrated. The residue was purified preparative HPLC (Phenomenex luna C18 10 μm, 150 mm×40 mm, water+0.1% FA-ACN) to give 6-[2-[2-[2-[2-[2-[2-(2-methoxyethoxy)ethoxy]ethoxy]ethoxy]ethoxy]ethoxy]ethoxy]pyridazin-3-amine (200 mg, 0.46 mmol, 35% yield) as colorless oil. LC-MS: m/z=434.1 [M+H]⁺, ESI pos.

### Building block E.3: 5-amino-2-methylpyridine

Error! Objects cannot be created from editing field codes.

### CAS: 3430-14-6 Building block E.4: 3-pyridazinamine

Error! Objects cannot be created from editing field codes.

### CAS: 5469-70-5 Building block E.5: 6-(2-methyl-1,3-dioxolan-2-yl)pyridazin-3-amine

Error! Objects cannot be created from editing field codes.

A mixture of 1-(6-aminopyridazin-3-yl)ethanone (930600-44-5) (40 mg, 0.29 mmol, 1.0 eq), ethylene glycol (0.05 mL, 0.88 mmol, 3.0 eq) and TosOH (5 mg, 0.03 mmol, 0.1 eq) in toluene (3 mL) was refluxed at 120° C. for 16 hours with azeotropical removal of water. The mixture was cooled to RT, concentrated and the residue was purified by preparative TLC (DCM/MeOH=10/1, Rf=0.4) to give 6-(2-methyl-1,3-dioxolan-2-yl)pyridazin-3-amine (20 mg, 0.11 mmol, 38% yield) as a white solid. LC-MS: m/z=182.2 [M+H]⁺, ESI pos. ¹H NMR (400 MHz, acetonitrile-d3) δ=7.38 (d, J=9.2 Hz, 1H), 6.79 (d, J=9.2 Hz, 1H), 5.21 (br s, 2H), 4.06-4.01 (m, 2H), 3.92-3.87 (m, 2H), 1.68 (s, 3H).

### Building block E.6: 3-amino-N,N,6-trimethyl-pyridazine-4-carboxamide

Error! Objects cannot be created from editing field codes.

### Step 1: ethyl 3-(benzhydrylideneamino)-6-methyl-pyridazine-4-carboxylate

To a solution of ethyl 3-chloro-6-methyl-pyridazine-4-carboxylate (1445-53-O) (1.0 g, 4.98 mmol, 1.0 eq) and benzophenone imine (1.25 mL, 7.48 mmol, 1.5 eq) in 1,4-dioxane (12 mL) were added Cs₂CO₃ (4.87 g, 14.95 mmol, 3.0 eq) and Xantphos Pd G4 (0.48 g, 0.5 mmol, 0.1 eq). The reaction mixture was stirred at 90° C. for 16 hours under N₂. The reaction mixture was cooled to RT, poured into water (100 mL) and extracted with EtOAc (3×100 mL). The combined organic layers were dried over Na₂SO₄, filtered and concentrated. The residue was purified by flash chromatography (SiO₂, 40% EtOAc in PE) to give 3-(benzhydrylideneamino)-6-methyl-pyridazine-4-carboxylic acid (800 mg, 2.52 mmol, 25% yield) as a yellow solid. LC-MS: m/z=346.1 [M+H]⁺, ESI pos. ¹H NMR (400 MHz, DMSO-d6) δ=7.79-7.05 (m, 11H), 4.23 (q, J=7.1 Hz, 2H), 2.56 (s, 3H), 1.16 (t, J=7.1 Hz, 3H).

### Step 2: lithium 3-(benzhydrylideneamino)-6-methyl-pyridazine-4-carboxylate

To a mixture of ethyl 3-(benzhydrylideneamino)-6-methyl-pyridazine-4-carboxylate (1.20 g, 3.47 mmol, 1.0 eq) in THF (6 mL), MeOH (6 mL) and H₂O (6 mL), was added LiOH (249 mg, 10.42 mmol, 3.0 eq), and the reaction was stirred at 30° C. for 12 hours. The reaction was concentrated to give lithium; 3-(benzhydrylideneamino)-6-methyl-pyridazine-4-carboxylate (1.00 g, 3.09 mmol, 89% yield) as a yellow solid which was used without further purification. LC-MS: m/z=318.1 [M+H]⁺, ESI pos.

### Step 3: 3-(benzhydrylideneamino)-N,N,6-trimethyl-pyridazine-4-carboxamide

To a suspension of dimethylamine hydrochloride (324 mg, 3.97 mmol, 1.5 eq) in DMF (10 mL) was added DIPEA (1.38 mL, 7.94 mmol, 3.0 eq), lithium 3-(benzhydrylideneamino)-6-methyl-pyridazine-4-carboxylate acid (840 mg, 2.65 mmol, 1.0 eq), HATU (934 mg, 3.97 mmol, 1.5 eq). The mixture was stirred at 30° C. for 1 hour. The reaction mixture was diluted with H₂O (50 mL) and extracted with EtOAc (3×20 mL). The combined organic layers were washed with brine (20 mL), dried over Na₂SO₄, filtered and concentrated. The residue was purified by flash chromatography (SiO₂, 30-40% EtOAc in PE) to give 3-(benzhydrylideneamino)-N,N,6-trimethyl-pyridazine-4-carboxamide (400 mg, 1.16 mmol, 39% yield) as a white solid. LC-MS: m/z=345.2 [M+H]⁺, ESI pos. ¹H NMR (400 MHz, CDCl₃) δ=7.84-7.22 (m, 11H), 3.06 (s, 3H), 3.02 (s, 3H), 2.61 (s, 3H).

### Step 4: 3-amino-N,N,6-trimethyl-pyridazine-4-carboxamide

To a solution of 3-(benzhydrylideneamino)-N,N,6-trimethyl-pyridazine-4-carboxamide (400 mg, 1.16 mmol, 1.0 eq) in MeOH (20 mL) was added hydroxylamine hydrochloride (161 mg, 2.32 mmol, 2.0 eq), NaOAc (238 mg, 2.9 mmol, 2.5 eq) and the mixture was stirred at 20° C. for 2 hours. The reaction mixture was concentrated. The residue was suspended in H₂O (20 mL) and extracted with EtOAc (3×10 mL). The combined organic layers were washed by brine (10 mL), dried over Na₂SO₄, filtered and concentrated. The residue was purified by preparative TLC (EtOAc) to give 3-amino-N,N,6-trimethyl-pyridazine-4-carboxamide (220 mg, 1.22 mmol, 95% yield) as brown solid. LC-MS: m/z=181.1 [M+H]⁺, ESI pos. ¹H NMR (400 MHz, CDCl₃) δ=6.92 (s, 1H), 5.42 (br s, 2H), 3.04 (br s, 3H), 2.94 (br s, 3H), 2.50 (s, 3H).

### Building block E.7: cyclopropanecarboxamide

Error! Objects cannot be created from editing field codes.

### CAS: 6228-73-5

### Building block E.8: 2,3-difluoro-4-methylbenzenamine

Error! Objects cannot be created from editing field codes.

### CAS: 886503-79-3

### Building block E.9: 2,3,4-trifluoroaniline

Error! Objects cannot be created from editing field codes.

### CAS: 3862-73-5

### Building block E.10: 3-amino-6-chloropyridazine

Error! Objects cannot be created from editing field codes.

### CAS: 5469-69-2

### Building block E.11: 2-amino-5-methyl-1,3,4-oxadiazole

Error! Objects cannot be created from editing field codes.

### CAS: 52838-39-8

### Building block E.12: 6,7-dihydro-5H-cyclopenta[c]pyridazin-3-amine

Error! Objects cannot be created from editing field codes.

### CAS:1342288-68-9

### Building block E.13: 2-amino-5-methyl-1,3,4-thiadiazole

Error! Objects cannot be created from editing field codes.

### CAS: 108-33-8

### Building block E.14: 5-fluoro-6-methyl-3-pyridinamine

Error! Objects cannot be created from editing field codes.

### CAS: 1211542-12-9

### Building block E.15: 5-(1-azetidinyl)-3-pyridazinamine

Error! Objects cannot be created from editing field codes.

### CAS: 1379254-05-3

### Building block E.16: 7,8-dihydro-5H-pyrano[4,3-c]pyridazin-3-amine

Error! Objects cannot be created from editing field codes.

### CAS: 1490340-46-9

### Building block E.17: 2-methyl-5-pyrimidinamine

Error! Objects cannot be created from editing field codes.

### CAS: 39889-94-6

### Building block E.18: 3-amino-1-methyl-1H-pyridin-2-one

Error! Objects cannot be created from editing field codes.

### CAS: 33631-01-5

### Building block E.19: 6-(difluoromethyl)pyridazin-3-amine

Error! Objects cannot be created from editing field codes.

### CAS: 1706450-11-4

| | | | Building | |
|---|---|---|---|---|
| Ex. | Structure | Systematic name | blocks | Analytics |
| 275 | Error! Objects cannot be | 1-[3-(1-hydroxyethyl)-6- | C.1 | LC-MS: m/z = 520.5 |
| | created from editing field | [5-[(6-methylpyridazin- | and | [M+H]+, ESI pos |
| | codes. | 3-yl)amino]-6- | D.1 | |
| | | (trifluoromethyl) | and | |
| | | benzimidazol- | E.1 | |
| | | 1-yl]-2-pyridyl]-5- | | |
| | | methyl-pyrazole-3- | | |
| | | carbonitrile | | |
| 282 | Error! Objects cannot be | 1-[3-(1-hydroxyethyl)-6- | C.2 | LC-MS: m/z = 524.0 |
| | created from editing field | [5-[(6-methylpyridazin- | and | [M+H]+, ESI pos |
| | codes. | 3-yl)amino]-6-(oxetan-3- | D.1 | ¹H NMR (400 MHz, |
| | | yloxy)benzimidazol-1- | and | METHANOL-d4) δ = |
| | | yl]-2-pyridyl]-5-methyl- | E.1 | 8.86 (s, 1H), 8.59 (s, 1H), |
| | | pyrazole-3-carbonitrile | | 8.50 (d, J = 8.6 Hz, 1H), |
| | | | | 8.15 (s, 1H), 7.47 (s, 1H), |
| | | | | 7.39 (d, J = 9.0 Hz, 1H), |
| | | | | 7.28 (d, J = 9.2 Hz, 1H), |
| | | | | 6.95 (s, 1H), 5.23 (quin, |
| | | | | J = 5.4 Hz, 1H), 4.83-4.77 |
| | | | | (m, 5H), 2.56 (s, 3H), |
| | | | | 2.41 (s, 3H), 1.43 (d, J = |
| | | | | 6.4 Hz, 3H) |
| 293 | Error! Objects cannot be | 1-[3-(1-hydroxyethyl)-6- | C.3 | LC-MS: m/z = 776.4 |
| | created from editing field | [5-[[6-[2- | and | [M+H]+, ESI pos |
| | codes. | [2-[2-[2-[2-[2-(2- | D.1 | ¹H NMR (400 MHz, |
| | | methoxyethoxy)ethoxy] | and | METHANOL-d4) δ ppm |
| | | ethoxy]ethoxy]ethoxy] | E.2 | 8.85-8.81(m, 1 H) 8.45- |
| | | ethoxy]ethoxy]pyridazin- | | 8.43 (m, J = 8.44 Hz, 1 |
| | | 3- | | H) 8.17-7.98 (m, 3 H) |
| | | yl]amino]benzimidazol- | | 7.52-7.48 (m, 1 H) 7.13 |
| | | 1-yl]- | | (br d, J = 9.41 Hz, 1 H) |
| | | 2-pyridyl]-5-methyl- | | 7.01-6.97 (m, 1 H) 6.85- |
| | | pyrazole-3-carbonitrile | | 6.84 (m, 1 H) 4.75 (br d, |
| | | | | J = 5.01 Hz, 1 H) 4.58 (br |
| | | | | s, 1 H) 4.46 (br d, J = |
| | | | | 3.30 Hz, 2 H) 3.86 (br s, |
| | | | | 2 H) 3.68-3.57 (m, 23 |
| | | | | H) 3.49 (br d, J = 3.91 |
| | | | | Hz, 3 H) 2.39-2.37 (m, 3 |
| | | | | H) 1.39 (br d, J = 5.99 |
| | | | | Hz, 3 H) |
| 294 | Error! Objects cannot be | 1-[3-(1-hydroxyethyl)-6- | C.4 | LC-MS: m/z = 580.2 |
| | created from editing field | [6-[(6-methyl-3- | and | [M+H]+, ESI pos |
| | codes. | pyridyl)amino]-5-(2- | D.1 | ¹H NMR (400 MHz, |
| | | morpholinoethoxy) | and | DMSO-d6) δ = 8.89 (s, |
| | | benzimidazol-1-yl]-2- | E.3 | 1H), 8.39 (d, J = 8.5 Hz, |
| | | pyridyl]- | | 1H), 8.26-8.22 (m, 1H), |
| | | 5-methyl-pyrazole-3- | | 8.22 (s, 1H), 8.21 (s, 1H), |
| | | carbonitrile | | 7.93 (s, 1H), 7.41 (s, 1H), |
| | | | | 7.37 (s, 1H), 7.32 (dd, J = |
| | | | | 2.8, 8.4 Hz, 1H), 7.00 (d, |
| | | | | J = 0.6 Hz, 1H), 6.94 (d, |
| | | | | J = 8.3 Hz, 1H), 5.45 (br |
| | | | | s, 1H), 4.48 (q, J = 6.5 |
| | | | | Hz, 1H), 4.21 (t, J = 5.8 |
| | | | | Hz, 2H), 3.55-3.52 (m, |
| | | | | 4H), 2.74 (t, J = 5.8 Hz, |
| | | | | 2H), 2.47-2.44 (m, 4H), |
| | | | | 2.43 (s, 3H), 2.16 (s, 3H), |
| | | | | 1.22 (d, J = 6.5 Hz, 3H). |
| 297 | Error! Objects cannot be | 1-[3-(1-hydroxyethyl)-6- | C.4 | LC-MS: m/z = 580.3 |
| | created from editing field | [5-[(6-methyl-3- | and | [M+H]+, ESI pos |
| | codes. | pyridyl)amino]-6-(2- | D.1 | ¹H NMR (400 MHz, |
| | | morpholinoethoxy) | and | DMSO-d6) δ = 8.89 (s, |
| | | benzimidazol-1-yl]-2- | E.3 | 1H), 8.45 (d, J = 8.5 Hz, |
| | | pyridyl]- | | 1H), 8.27 (d, J = 8.5 Hz, |
| | | 5-methyl-pyrazole-3- | | 1H), 8.19 (d, J = 2.8 Hz, |
| | | carbonitrile | | 1H), 7.80 (s, 1H), 7.44- |
| | | | | 7.38 (m, 2H), 7.28 (dd, |
| | | | | J = 2.8, 8.3 Hz, 1H), 7.10- |
| | | | | 7.04 (m, 2H), 5.54 (br s, |
| | | | | 1H), 4.61 (br d, J = 6.0 |
| | | | | Hz, 1H), 4.06 (t, J = 5.9 |
| | | | | Hz, 2H), 3.58-3.47 (m, |
| | | | | 4H), 2.65 (t, J = 5.9 Hz, |
| | | | | 2H), 2.39-2.34 (m, |
| | | | | 10H), 1.28 (d, J = 6.4 Hz, |
| | | | | 3H). |
| 302 | Error! Objects cannot be | 1-[6-[6-fluoro-5-[(6- | C.5 | LC-MS: m/z = 527.1 |
| | created from editing field | methylpyridazin-3- | and | [M+H]+, ESI pos |
| | codes. | yl)amino]benzimidazol- | D.2 | ¹H NMR (400 MHz, |
| | | 1-yl]-2-[3-methyl-1- | and | METHANOL-d4) δ = |
| | | (2,2,2- | E.1 | 8.88 (s, 1H), 8.49 (d, J = |
| | | trifluoroethyl)pyrazol-4- | | 7.5 Hz, 1H), 8.36 (br dd, |
| | | yl]-3-pyridyl]ethanol | | J = 2.3, 4.9 Hz, 1H), 8.29 |
| | | | | (d, J = 8.5 Hz, 1H), 8.11 |
| | | | | (d, J = 11.4 Hz, 1H), 8.02 |
| | | | | (s, 1H), 7.86 (d, J = 8.5 |
| | | | | Hz, 1H), 7.38 (d, J = 9.1 |
| | | | | Hz, 1H), 7.20 (d, J = 9.1 |
| | | | | Hz, 1H), 5.03-4.95 (m, |
| | | | | 3H), 2.54 (s, 3H), 2.36 (s, |
| | | | | 3H), 1.47 (d, J = 6.4 Hz, |
| | | | | 3H) |
| 307 | Error! Objects cannot be | (1S)-1-[2-[3- | C.5 | LC-MS: m/z = 481.1 |
| | created from editing field | (difluoromethyl)-5- | and | [M+H]+, ESI pos |
| | codes. | methyl-pyrazol-1-yl]-6- | D.3 | |
| | | [6-fluoro-5-(pyridazin-3- | and | |
| | | ylamino)benzimidazol-1- | E.4 | |
| | | yl]-3-pyridyl]ethanol | | |
| 314 | Error! Objects cannot be | 1-[6-[5-fluoro-6-[(6- | C.5 | LC-MS: m/z = 456.0 |
| | created from editing field | methylpyridazin-3- | and | [M+H]+, ESI pos |
| | codes. | yl)amino]benzimidazol- | D.6 | ¹H NMR (400 MHz, |
| | | 1-yl]-3-(hydroxymethyl)- | and | METHANOL-d4) δ ppm |
| | | 2-pyridyl]-5-methyl- | E.1 | 9.02-9.09 (m, 1 H) 8.84- |
| | | pyrazole-3-carbonitrile | | 8.93 (m, 1 H) 8.50- |
| | | | | 8.54 (m, 1 H) 8.12-8.16 |
| | | | | (m, 1 H) 7.56 (s, 1 H) |
| | | | | 7.38-7.42 (m, 1 H) 7.23- |
| | | | | 7.28 (m, 1 H) 6.78- |
| | | | | 6.82 (m, 1 H) 4.61-4.62 |
| | | | | (m, 2 H) 2.57-2.60 (m, 3 |
| | | | | H) 2.41-2.44 (m, 3 H) |
| 317 | Error! Objects cannot be | [2-[3-(difluoromethyl)-5- | C.3 | LC-MS: m/z = 505.2 |
| | created from editing field | methyl-pyrazol-1-yl]-6- | and | [M+H]+, ESI pos |
| | codes. | [5-(7,8-dihydro-5H- | D.7 | |
| | | pyrano[4,3-c]pyridazin- | and | |
| | | 3-ylamino)benzimidazol- | E.16 | |
| | | 1-yl]-3-pyridyl]methanol | followed | |
| | | | by | |
| | | | reduction | |
| | | | in | |
| | | | analogy | |
| | | | to | |
| | | | Example | |
| | | | 1, step 3 | |
| 319 | Error! Objects cannot be | [2-[3-(difluoromethyl)-5- | C.5 | LC-MS: m/z = 481.1 |
| | created from editing field | methyl-pyrazol-1-yl]-6- | and | [M+H]+, ESI pos |
| | codes. | [6-fluoro-5-[(6- | D.7 | ¹H NMR (400 MHz, |
| | | methylpyridazin-3- | and | METHANOL-d4) δ ppm |
| | | yl)amino]benzimidazol- | E.1 | 8.93 (s, 1 H) 8.54-8.58 |
| | | 1-yl]-3-pyridyl]methanol | followed | (d, 1 H) 8.42-8.46 (d, 1 |
| | | | by | H) 8.02-8.11 (m, 2 H) |
| | | | reduction | 7.37-7.42 (m, 1 H) 7.21- |
| | | | in | 7.25 (m, 1 H) 6.68- |
| | | | analogy | 6.98 (m, 1 H) 6.59-6.64 |
| | | | to | (m, 1 H) 4.59-4.61 (m, 2 |
| | | | Example | H) 2.56 (s, 3 H) 2.47 (s, 3 |
| | | | 1, step 3 | H) |
| 333 | Error! Objects cannot be | 1-[3-(1-hydroxyethyl)-6- | C.3 | LC-MS: m/z = 467.5 |
| | created from editing field | [5-[(2-keto-1-methyl-3- | and | [M+H]+, ESI pos |
| | codes. | pyridyl)amino]benzimidazol- | D.1 | ¹H NMR (300 MHZ, |
| | | 1-yl]-2-pyridyl]-5- | and | DMSO-d6) δ = 9.03 (s, |
| | | methyl-pyrazole-3- | E.18 | 1H), 8.45 (d, J = 8.5 Hz, |
| | | carbonitrile | | 1H), 8.27 (d, J = 8.7 Hz, |
| | | | | 1H), 8.04 (d, J = 8.9 Hz, |
| | | | | 1H), 7.72 (s, 1H), 7.60 (d, |
| | | | | J = 2.0 Hz, 1H), 7.31 (dd, |
| | | | | J = 2.0, 8.9 Hz, 1H), 7.14- |
| | | | | 7.04 (m, 3H), 6.16 (t, |
| | | | | J = 7.1 Hz, 1H), 5.53 (d, |
| | | | | J = 4.4 Hz, 1H), 4.56 (br |
| | | | | dd, J = 4.1, 6.1 Hz, 1H), |
| | | | | 3.53 (s, 3H), 2.37-2.32 |
| | | | | (m, 3H), 1.26 (d, J = 6.2 |
| | | | | Hz, 3H) |
| 337 | Error! Objects cannot be | 1-[2-[3-(difluoromethyl)- | C.5 | LC-MS: m/z = 567.2 |
| | created from editing field | 5-methyl-pyrazol-1-yl]-6- | and | [M+H]+, ESI pos |
| | codes. | [6-fluoro-5-[[6-(2- | D.3 | ¹H NMR (400 MHz, |
| | | methyl-1,3-dioxolan-2- | and | METHANOL-d4) δ = |
| | | yl)pyridazin-3- | E.5 | 8.95 (s, 1H), 8.49 (dd, J = |
| | | yl]amino]benzimidazol- | | 4.0, 7.9 Hz, 2H), 8.12 (d, |
| | | 1-yl]-3-pyridyl]ethanol | | J = 8.3 Hz, 1H), 8.06 (d, |
| | | | | J = 11.1 Hz, 1H), 7.62 (d, |
| | | | | J = 9.3 Hz, 1H), 7.23 (d, |
| | | | | J = 9.3 Hz, 1H), 6.99- |
| | | | | 6.65 (m, 1H), 6.60 (s, |
| | | | | 1H), 4.83 (d, J = 6.6 Hz, |
| | | | | 1H), 4.13-4.08 (m, 2H), |
| | | | | 3.93-3.89 (m, 2H), 2.41 |
| | | | | (s, 3H), 1.72 (s, 3H), 1.39 |
| | | | | (d, J = 6.5 Hz, 3H) |
| 345 | Error! Objects cannot be | 3-[[1-[6-[3- | C.5 | LC-MS: m/z = 566.2 |
| | created from editing field | (difluoromethyl)-5- | and | [M+H]+, ESI pos |
| | codes. | methyl-pyrazol-1-yl]-5- | D.3 | ¹H NMR (400 MHz, |
| | | [(1~{S})-1- | and | METHANOL-d4) δ = |
| | | hydroxyethyl]-2- | E.6 | 8.83 (s, 1H), 8.41-8.35 |
| | | pyridyl]-6-fluoro- | | (m, 1H), 8.22-8.16 (m, |
| | | benzimidazol-5- | | 1H), 8.04-7.98 (m, 1H), |
| | | yl]amino]-~{N},~{N},6- | | 7.95-7.90 (m, 1H), 7.31 |
| | | trimethyl-pyridazine-4- | | (s, 1H), 6.86-6.54 (t, |
| | | carboxamide | | 1H), 6.49 (s, 1H), 4.73- |
| | | | | 4.69 (m, 1H), 3.03-3.00 |
| | | | | (m, 3H), 2.98 (s, 3H), |
| | | | | 2.48 (s, 3H), 2.30 (s, 3H), |
| | | | | 1.30-1.26 (d, 3H) |
| 347 | Error! Objects cannot be | 1-[6-[6-fluoro-5-[(6- | C.5 | LC-MS: m/z = 456.0 |
| | created from editing field | methylpyridazin-3- | and | [M+H]+, ESI pos |
| | codes. | yl)amino]benzimidazol- | D.6 | ¹H NMR (400 MHz, |
| | | 1-yl]-3-(hydroxymethyl)- | and | DMSO-d6) δ = 9.07 (s, |
| | | 2-pyridyl]-5-methyl- | E.1 | 1H), 8.87 (s, 1H), 8.69- |
| | | pyrazole-3-carbonitrile | | 8.64 (m, 1H), 8.44-8.39 |
| | | | | (m, 1H), 8.31-8.26 (m, |
| | | | | 1H), 8.02-7.97 (m, 1H), |
| | | | | 7.38-7.33 (m, 1H), 7.27- |
| | | | | 7.23 (m, 1H), 7.11- |
| | | | | 7.08 (m, 1H), 5.58-5.48 |
| | | | | (m, 1H), 4.45-4.40 (m, |
| | | | | 2H), 2.49-2.48 (s, 3H), |
| | | | | 2.40-2.37 (s, 3H) |
| 348 | Error! Objects cannot be | 3-[[1-[6-[3,5- | C.3 | LC-MS: m/z = 570.2 |
| | created from editing field | bis(difluoromethyl)pyraz | and | [M+H]+, ESI pos |
| | codes. | ol-1-yl]-5- | D.8 | ¹H NMR (400 MHz, |
| | | (hydroxymethyl)-2- | and | METHANOL-d4) δ = |
| | | pyridyl]benzimidazol-5- | E.6 | 8.91-8.88 (m, 1H), 8.54- |
| | | yl]amino]-N,N,6- | | 8.49 (m, 1H), 8.11- |
| | | trimethyl-pyridazine-4- | | 8.05 (m, 3H), 7.61-7.57 |
| | | carboxamide | | (m, 1H), 7.47-7.18 (m, |
| | | | | 2H), 7.17-7.12 (m, 1H), |
| | | | | 7.10-6.81 (m, 1H), 4.79- |
| | | | | 4.77 (m, 2H), 3.12- |
| | | | | 3.09 (m, 3H), 3.08-3.05 |
| | | | | (m, 3H), 2.61-2.58 (m, |
| | | | | 3H) |
| 353 | Error! Objects cannot be | 1-[2-[3- | C.2 | LC-MS: m/z = 565.2 |
| | created from editing field | (difluoromethoxy)-5- | and | [M+H]+, ESI pos |
| | codes. | methyl-pyrazol-1-yl]-6- | D.4 | ¹H NMR (400 MHz, |
| | | [6-[(6-methylpyridazin- | and | METHANOL-d4) δ = |
| | | 3-yl)amino]-5-(oxetan-3- | E.1 | 8.99 (s, 1H), 8.71 (s, 1H), |
| | | yloxy)benzimidazol-1- | | 8.48 (d, J = 8.4 Hz, 1H), |
| | | yl]-3-pyridyl]ethanol | | 7.99 (d, J = 8.4 Hz, 1H), |
| | | | | 7.38-7.26 (m, 2H), 7.23 |
| | | | | (s, 1H), 7.05 (s, 1H), 6.90 |
| | | | | (s, 1H), 6.87 (s, 1H), 5.99 |
| | | | | (s, 1H), 5.40 (quin, J = |
| | | | | 5.4 Hz, 1H), 5.08 (t, J = |
| | | | | 6.8 Hz, 2H), 4.97 (q, J = |
| | | | | 6.4 Hz, 1H), 4.80 (dd, J = |
| | | | | 5.0, 7.4 Hz, 2H), 2.55 (s, |
| | | | | 3H), 2.33 (s, 3H), 1.41 (d, |
| | | | | J = 6.4 Hz, 3H). |
| 354 | Error! Objects cannot be | 1-[2-(3-chloro-5-methyl- | C.5 | LC-MS: m/z = 479.1 |
| | created from editing field | pyrazol-1-yl)-6-[5-fluoro- | and | [M+H]+, ESI pos |
| | codes. | 6-[(6-methylpyridazin-3- | D.5 | ¹H NMR (400 MHz, |
| | | yl)amino]benzimidazol- | and | METHANOL-d4) δ = |
| | | 1-yl]-3-pyridyl]ethanol | E.1 | 8.99 (d, J = 7.3 Hz, 1H), |
| | | | | 8.87 (s, 1H), 8.54 (d, J = |
| | | | | 8.4 Hz, 1H), 8.11 (d, J = |
| | | | | 8.4 Hz, 1H), 7.58 (d, J = |
| | | | | 10.9 Hz, 1H), 7.40 (d, J = |
| | | | | 9.0 Hz, 1H), 7.26 (d, J = |
| | | | | 9.0 Hz, 1H), 6.30 (s, 1H), |
| | | | | 4.94-4.91 (m, 1H), 2.58 |
| | | | | (s, 3H), 2.34 (s, 3H), 1.42 |
| | | | | (d, J = 6.5 Hz, 3H) |
| 355 | Error! Objects cannot be | 1-[2-(3-chloro-5-methyl- | C.5 | LC-MS: m/z = 479.2 |
| | created from editing field | pyrazol-1-yl)-6-[6-fluoro- | and | [M+H]+, ESI pos |
| | codes. | 5-[(6-methylpyridazin-3- | D.5 | ¹H NMR (400 MHz, |
| | | yl)amino]benzimidazol- | and | METHANOL-d4) δ = |
| | | 1-yl]-3-pyridyl]ethanol | E.1 | 8.94 (s, 1H), 8.56 (d, J = |
| | | | | 7.6 Hz, 1H), 8.49 (d, J = |
| | | | | 8.6 Hz, 1H), 8.14-7.99 |
| | | | | (m, 2H), 7.40 (d, J = 9.0 |
| | | | | Hz, 1H), 7.23 (d, J = 9.0 |
| | | | | Hz, 1H), 6.39 (s, 1H), |
| | | | | 4.94-4.92 (m, 1H), 2.56 |
| | | | | (s, 3H), 2.38 (s, 3H), 1.42 |
| | | | | (d, J = 6.5 Hz, 3H) |
| 357 | Error! Objects cannot be | 1-[2-[3- | C.2 | LC-MS: m/z = 565.1 |
| | created from editing field | (difluoromethoxy)-5- | and | [M+H]+, ESI pos |
| | codes. | methyl-pyrazol-1-yl]-6- | D.4 | ¹H NMR (400 MHz, |
| | | [5-[(6-methylpyridazin- | and | METHANOL-d4) δ = |
| | | 3-yl)amino]-6-(oxetan-3- | E.1 | 8.84 (s, 1H), 8.57 (s, 1H), |
| | | yloxy)benzimidazol-1- | | 8.55 (s, 1H), 8.43 (d, J = |
| | | yl]-3-pyridyl]ethanol | | 8.5 Hz, 1H), 8.05 (d, J = |
| | | | | 8.4 Hz, 1H), 7.54 (s, 1H), |
| | | | | 7.37 (d, J = 9.1 Hz, 1H), |
| | | | | 7.28-7.24 (m, 1H), 7.24- |
| | | | | 6.89 (m, 1H), 6.13 (s, |
| | | | | 1H), 5.25 (quin, J = 5.5 |
| | | | | Hz, 1H), 4.95 (q, J = 6.4 |
| | | | | Hz, 1H), 4.83 (br s, 1H), |
| | | | | 4.79-4.73 (m, 2H), 4.58 |
| | | | | (br s, 1H), 2.54 (s, 3H), |
| | | | | 2.36 (s, 3H), 1.43 (d, J = |
| | | | | 6.4 Hz, 3H). |
| 361 | Error! Objects cannot be | N-[1-[6-[3- | C.5 | LC-MS: m/z = 529.2 |
| | created from editing field | (difluoromethyl)-5- | and | [M+H]+, ESI pos |
| | codes. | methyl-pyrazol-1-yl]-5- | D.3 | ¹H NMR (300 MHz, |
| | | (1-hydroxyethyl)-2- | and | DMSO-d6) d = 10.02 (s, |
| | | pyridyl]-6-fluoro- | E.7 | 1H), 9.07 (s, 1H), 8.44 (d, |
| | | benzimidazol-5- | | J = 8.5 Hz, 1H), 8.24 (d, |
| | | yl] | | J = 8.3 Hz, 2H), 7.97 (d, |
| | | cyclopropanecarboxamide | | J = 11.1 Hz, 1H), 7.07 (t, |
| | | | | J = 54.5 Hz, 1H), 6.67 (d, |
| | | | | J = 0.6 Hz, 1H), 5.50 (s, |
| | | | | 1H), 4.79-4.58 (m, 1H), |
| | | | | 2.34 (s, 3H), 2.14-1.91 |
| | | | | (m, 1H), 1.23 (d, J = 6.4 |
| | | | | Hz, 3H), 0. 95-0.72 (m, |
| | | | | 4H) |
| 362 | Error! Objects cannot be | 1-[6-[6-(2,3-difluoro-4- | C.5 | LC-MS: m/z = 529.2 |
| | created from editing field | methyl-anilino)-5-fluoro- | and | [M+H]+, ESI pos |
| | codes. | benzimidazol-1-yl]-2-[3- | D.3 | |
| | | (difluoromethyl)-5- | and | |
| | | methyl-pyrazol-1-yl]-3- | E.8 | |
| | | pyridyl]ethanol | | |
| 364 | Error! Objects cannot be | 1-[2-[3-(difluoromethyl)- | C.5 | LC-MS: m/z = 533.2 |
| | created from editing field | 5-methyl-pyrazol-1-yl]-6- | and | [M+H]+, ESI pos |
| | codes. | [5-fluoro-6-(2,3,4- | D.3 | |
| | | trifluoroanilino) | and | |
| | | benzimidazol- | E.9 | |
| | | 1-yl]-3- | | |
| | | pyridyl]ethanol | | |
| 367 | Error! Objects cannot be | 1-[2-[3- | C.3 | LC-MS: m/z = 529.2 |
| | created from editing field | (difluoromethoxy)-5- | and | [M+H]+, ESI pos |
| | codes. | methylpyrazol-1-yl]-6-[5- | D.4 | |
| | | [[6- | and | |
| | | (difluoromethyl)pyridazin-3- | E.19 | |
| | | yl]amino]benzimidazol- | | |
| | | 1-yl]pyridin-3-yl]ethanol | | |
| 371 | Error! Objects cannot be | 1-[6-[6-[(6- | C.5 | LC-MS: m/z = 515.2 |
| | created from editing field | chloropyridazin-3- | and | [M+H]+, ESI pos |
| | codes. | yl)amino]-5-fluoro- | D.3 | |
| | | benzimidazol-1-yl]-2-[3- | and | |
| | | (difluoromethyl)-5- | E.10 | |
| | | methyl-pyrazol-1-yl]-3- | | |
| | | pyridyl]ethanol | | |
| 372 | Error! Objects cannot be | 1-[6-[5-[(6- | C.5 | LC-MS: m/z = 515.2 |
| | created from editing field | chloropyridazin-3- | and | [M+H]+, ESI pos |
| | codes. | yl)amino]-6-fluoro- | D.3 | |
| | | benzimidazol-1-yl]-2-[3- | and | |
| | | (difluoromethyl)-5- | E.10 | |
| | | methyl-pyrazol-1-yl]-3- | | |
| | | pyridyl]ethanol | | |
| 373 | Error! Objects cannot be | (1S)-1-[2-[3- | C.5 | LC-MS: m/z = 497.15 |
| | created from editing field | (difluoromethoxy)-5- | and | [M+H]+, ESI pos |
| | codes. | methyl-pyrazol-1-yl]-6- | D.4 | ¹H NMR (300 MHz, |
| | | [6-fluoro-5-(pyridazin-3- | and | DMSO-d6) δ = 9.07 (s, |
| | | ylamino)benzimidazol-1- | E.4 | 1H), 9.00 (s, 1H), 8.68 |
| | | yl]-3-pyridyl]ethanol | | (dd, J = 1.3, 4.5 Hz, 1H), |
| | | | | 8.57 (d, J = 7.7 Hz, 1H), |
| | | | | 8.42 (d, J = 8.5 Hz, 1H), |
| | | | | 8.21 (d, J = 8.5 Hz, 1H), |
| | | | | 8.03 (d, J = 11.5Hz, 1H), |
| | | | | 7.51-7.42 (m, 1H), 7.27 |
| | | | | (dd, J = 1.2, 9.1 Hz, 1H), |
| | | | | 7.36 (t, J = 73.1 Hz, 1H), |
| | | | | 6.21 (s, 1H), 5.47 (d, J = |
| | | | | 4.4 Hz, 1H), 4.88-4.72 |
| | | | | (m, 1H), 2.34 (d, J = 0.6 |
| | | | | Hz, 3H), 1.27 (d, J = 6.2 |
| | | | | Hz, 3H) |
| 374 | Error! Objects cannot be | (1S)-1-[2-[3- | C.5 | LC-MS: m/z = 511.17 |
| | created from editing field | (difluoromethoxy)-5- | and | [M+H]+, ESI pos |
| | codes. | methyl-pyrazol-1-yl]-6- | D.4 | ¹H NMR (300 MHz, |
| | | [6-fluoro-5-[(6- | and | DMSO-d6) δ = 9.05 (s, |
| | | methylpyridazin-3- | E.1 | 1H), 8.85 (d, J = 1.6 Hz, |
| | | yl)amino]benzimidazol- | | 1H), 8.65 (d, J = 7.9 Hz, |
| | | 1-yl]-3-pyridyl]ethanol | | 1H), 8.41 (d, J = 8.5 Hz, |
| | | | | 1H), 8.20 (d, J = 8.5 Hz, |
| | | | | 1H), 8.01 (d, J = 11.5 Hz, |
| | | | | 1H), 7.28-7.21 (m, 1H), |
| | | | | 7.63-7.08 (m, 2H), 6.21 |
| | | | | (s, 1H), 5.46 (d, J = 4.4 |
| | | | | Hz, 1H), 4.89-4.67 (m, |
| | | | | 1H), 2.48 (br s, 3H), 2.33 |
| | | | | (d, J = 0.6 Hz, 3H), 1.27 |
| | | | | (d, J = 6.4 Hz, 3H) |
| 375 | Error! Objects cannot be | (1R)-1-[2-[3- | C.5 | LC-MS: m/z = 511.14 |
| | created from editing field | (difluoromethoxy)-5- | and | [M+H]+, ESI pos |
| | codes. | methyl-pyrazol-1-yl]-6- | D.4 | ¹H NMR (300 MHz, |
| | | [6-fluoro-5-[(6- | and | DMSO-d6) δ = 9.05 (s, |
| | | methylpyridazin-3- | E.1 | 1H), 8.85 (d, J = 1.6 Hz, |
| | | yl)amino]benzimidazol- | | 1H), 8.65 (d, J = 7.9 Hz, |
| | | 1-yl]-3-pyridyl]ethanol | | 1H), 8.41 (d, J = 8.5 Hz, |
| | | | | 1H), 8.20 (d, J = 8.5 Hz, |
| | | | | 1H), 8.01 (d, J = 11.5 Hz, |
| | | | | 1H), 7.28-7.21 (m, 1H), |
| | | | | 7.67-7.09 (m, 2H), 6.21 |
| | | | | (s, 1H), 5.46 (d, J = 4.4 |
| | | | | Hz, 1H), 4.88-4.70 (m, |
| | | | | 1H), 2.48 (br s, 3H), 2.33 |
| | | | | (d, J = 0.6 Hz, 3H), 1.27 |
| | | | | (d, J = 6.4 Hz, 3H) |
| 376 | Error! Objects cannot be | 1-[6-[5-fluoro-6-[(5- | C.5 | LC-MS: m/z = 460.15 |
| | created from editing field | methyl-1,3,4-oxadiazol- | and | [M+H]+, ESI pos |
| | codes. | 2-yl)amino]benzimidazol- | D.1 | ¹H NMR (300 MHz, |
| | | 1-yl]-3-(1-hydroxyethyl)- | and | DMSO-d6) δ = 10.19 (s, |
| | | 2-pyridyl]-5-methyl- | E.11 | 1H), 9.04 (s, 1H), 8.92 (d, |
| | | pyrazole-3-carbonitrile | | J = 7.7 Hz, 1H), 8.48 (d, |
| | | | | J = 8.5 Hz, 1H), 8.26 (d, |
| | | | | J = 8.5 Hz, 1H), 7.72 (d, |
| | | | | J = 11.3 Hz, 1H), 7.03 (d, |
| | | | | J = 0.8 Hz, 1H), 5.54 (d, |
| | | | | J = 4.2 Hz, 1H), 4.54 (dd, |
| | | | | J = 4.4, 6.4 Hz, 1H), 2.42 |
| | | | | (d, J = 0.6 Hz, 3H), 2.40 |
| | | | | (s, 3H), 1.26 (d, J = 6.4 |
| | | | | Hz, 3H) |
| 377 | Error! Objects cannot be | 1-[6-[6-fluoro-5-[(5- | C.5 | LC-MS: m/z = 460.15 |
| | created from editing field | methyl-1,3,4-oxadiazol- | and | [M+H]+, ESI pos |
| | codes. | 2- | D.1 | ¹H NMR (300 MHz, |
| | | yl)amino]benzimidazol- | and | DMSO-d6) δ = 10.18 (s, |
| | | 1-yl]-3-(1-hydroxyethyl)- | E.11 | 1H), 9.09 (s, 1H), 8.47 (d, |
| | | 2-pyridyl]-5-methyl- | | J = 8.5 Hz, 1H), 8.42 (d, |
| | | pyrazole-3-carbonitrile | | J = 7.9 Hz, 1H), 8.30 (d, |
| | | | | J = 8.7 Hz, 1H), 7.98 (d, |
| | | | | J = 11.3 Hz, 1H), 7.11 (d, |
| | | | | J = 0.8 Hz, 1H), 5.55 (d, |
| | | | | J = 4.6 Hz, 1H), 4.58 (dd, |
| | | | | J = 4.5, 6.3 Hz, 1H), 2.41 |
| | | | | (s, 3H), 2.35 (s, 3H), 1.26 |
| | | | | (d, J = 6.4 Hz, 3H) |
| 378 | Error! Objects cannot be | 1-[6-[6-fluoro-5-(2,3,4- | C.5 | LC-MS: m/z = 508.16 |
| | created from editing field | trifluoroanilino) | and | [M+H]+, ESI pos |
| | codes. | benzimidazol-1-yl]-3-(1- | D.1 | ¹H NMR (300 MHz, |
| | | hydroxyethyl)-2- | and | DMSO-d6) d = 9.06 (s, |
| | | pyridyl]-5-methyl- | E.9 | 1H), 8.40 (d, J = 8.7 Hz, |
| | | pyrazole-3-carbonitrile | | 1H), 8.26 (d, J = 8.7 Hz, |
| | | | | 1H), 8.01 (s, 1H), 7.72 |
| | | | | (dd, J = 2.4, 8.1 Hz, 1H), |
| | | | | 7.65 (d, J = 11.3 Hz, 1H), |
| | | | | 7.18 (br d, J = 9.7 Hz, |
| | | | | 1H), 6.98-6.93 (m, 1H), |
| | | | | 6.92 (s, 1H), 5.48 (s, 1H), |
| | | | | 4.48 (br d, J = 6.6 Hz, |
| | | | | 1H), 2.11 (d, J = 0.6 Hz, |
| | | | | 3H), 1.21 (d, J = 6.4 Hz, |
| | | | | 3H) |
| 379 | Error! Objects cannot be | 1-[6-[5-fluoro-6-(2,3,4- | C.5 | LC-MS: m/z = 508.16 |
| | created from editing field | trifluoroanilino) | and | [M+H]+, ESI pos |
| | codes. | benzimidazol-1-yl]-3-(1- | D.1 | ¹H NMR (300 MHz, |
| | | hydroxyethyl)-2- | and | DMSO-d6) d = 9.07 (s, |
| | | pyridyl]-5-methyl- | E.9 | 1H), 8.47 (d, J = 8.5 Hz, |
| | | pyrazole-3-carbonitrile | | 1H), 8.29 (d, J = 8.5 Hz, |
| | | | | 1H), 8.00 (s, 1H), 7.96 (d, |
| | | | | J = 11.3 Hz, 1H), 7.43 |
| | | | | (dd, J = 0.9, 7.8 Hz, 1H), |
| | | | | 7.15 (br d, J = 9.5 Hz, |
| | | | | 1H), 7.11 (d, J = 0.8 Hz, |
| | | | | 1H), 6.78-6.64 (m, 1H), |
| | | | | 5.55 (br s, 1H), 4.57 (d, |
| | | | | J = 6.4 Hz, 1H), 2.35 (d, |
| | | | | J = 0.6 Hz, 3H), 1.26 (d, |
| | | | | J = 6.4 Hz, 3H) |
| 381 | Error! Objects cannot be | 1-[6-[6-(6,7-dihydro- | C.5 | LC-MS: m/z = 496.24 |
| | created from editing field | 5~{H}- | and | [M+H]+, ESI pos |
| | codes. | cyclopenta[c]pyridazin- | D.1 | ¹H NMR (300 MHz, |
| | | 3-ylamino)-5-fluoro- | and | DMSO-d6) d = 9.00 (s, |
| | | benzimidazol-1-yl]-3-(1- | E.12 | 1H), 9.00-8.96 (m, 1H), |
| | | hydroxyethyl)-2- | | 8.78 (d, J = 1.4 Hz, 1H), |
| | | pyridyl]-5-methyl- | | 8.47 (d, J = 8.5 Hz, 1H), |
| | | pyrazole-3-carbonitrile | | 8.24 (d, J = 8.5 Hz, 1H), |
| | | | | 7.68 (d, J = 11.3 Hz, 1H), |
| | | | | 7.10 (s, 1H), 6.99 (d, J = |
| | | | | 0.8 Hz, 1H), 5.54 (br s, |
| | | | | 1H), 4.55 (d, J = 6.4 Hz, |
| | | | | 1H), 3.02-2.93 (m, 2H), |
| | | | | 2.85 (t, J = 7.4 Hz, 2H), |
| | | | | 2.73 (s, 1H), 2.34 (s, 3H), |
| | | | | 1.26 (d, J = 6.2 Hz, 4H) |
| 382 | Error! Objects cannot be | 1-[6-[5-(6,7-dihydro- | C.5 | LC-MS: m/z = 496.24 |
| | created from editing field | 5~{H}- | and | [M+H]+, ESI pos |
| | codes. | cyclopenta[c]pyridazin- | D.1 | ¹H NMR (300 MHz, |
| | | 3-ylamino)-6-fluoro- | and | DMSO-d6) δ = 9.07 (s, |
| | | benzimidazol-1-yl]-3-(1- | E.12 | 1H), 8.75 (d, J = 1.4 Hz, |
| | | hydroxyethyl)-2- | | 1H), 8.62 (d, J = 7.9 Hz, |
| | | pyridyl]-5-methyl- | | 1H), 8.47 (d, J = 8.5 Hz, |
| | | pyrazole-3-carbonitrile | | 1H), 8.31 (d, J = 8.7 Hz, |
| | | | | 1H), 7.96 (d, J = 11.5 Hz, |
| | | | | 1H), 7.13 (s, 1H), 7.11 (d, |
| | | | | J = 0.8 Hz, 1H), 5.55 (br |
| | | | | s, 1H), 4.63-4.51 (m, |
| | | | | 1H), 3.01-2.82 (m, 4H), |
| | | | | 2.36 (d, J = 0.6 Hz, 3H), |
| | | | | 2.09-1.94 (m, 2H), 1.26 |
| | | | | (d, J = 6.4 Hz, 3H) |
| 383 | Error! Objects cannot be | (1S)-1-[2-[3- | C.2 | LC-MS: m/z = 565.3 |
| | created from editing field | (difluoromethoxy)-5- | and | [M+H]+, ESI pos |
| | codes. | methyl-pyrazol-1-yl]-6- | D.4 | ¹H NMR (400 MHz, |
| | | [5-[(6-methylpyridazin- | and | METHANOL-d4) δ = |
| | | 3-yl)amino]-6-(oxetan-3- | E.1 | 8.86 (s, 1H), 8.58 (s, 1H), |
| | | yloxy)benzimidazol-1- | | 8.46 (d, J = 8.4 Hz, 1H), |
| | | yl]-3-pyridyl]ethanol | | 8.07 (d, J = 8.4 Hz, 1H), |
| | | | | 7.57 (s, 1H), 7.40 (d, J = |
| | | | | 9.3 Hz, 1H), 7.31-6.89 |
| | | | | (m, 2H), 6.15 (s, 1H), |
| | | | | 5.27 (t, J = 5.3 Hz, 1H), |
| | | | | 4.97 (q, J = 6.3 Hz, 2H), |
| | | | | 4.80-4.76 (m, 3H), 2.56 |
| | | | | (s, 3H), 2.38 (s, 3H), 1.45 |
| | | | | (d, J = 6.5 Hz, 3H) |
| 384 | Error! Objects cannot be | (1R)-1-[2-[3- | C.2 | LC-MS: m/z = 565.0 |
| | created from editing field | (difluoromethoxy)-5- | and | [M+H]+, ESI pos |
| | codes. | methyl-pyrazol-1-yl]-6- | D.4 | ¹H NMR (400 MHz, |
| | | [5-[(6-methylpyridazin- | and | METHANOL-d4) δ = |
| | | 3-yl)amino]-6-(oxetan-3- | E.1 | 8.85 (s, 1H), 8.59 (s, 1H), |
| | | yloxy)benzimidazol-1- | | 8.45 (d, J = 8.4 Hz, 1H), |
| | | yl]-3-pyridyl]ethanol | | 8.07 (d, J = 8.6 Hz, 1H), |
| | | | | 7.56 (s, 1H), 7.39 (d, J = |
| | | | | 9.2 Hz, 1H), 7.30-6.89 |
| | | | | (m, 2H), 6.15 (s, 1H), |
| | | | | 5.26 (quin, J = 5.5 Hz, |
| | | | | 1H), 5.00-4.90 (m, 2H), |
| | | | | 4.85 (br s, 1H), 4.81- |
| | | | | 4.76 (m, 2H), 2.56 (s, |
| | | | | 3H), 2.37 (s, 3H), 1.45 (d, |
| | | | | J = 6.4 Hz, 3H) |
| 385 | Error! Objects cannot be | 1-[6-[6-fluoro-5-[(5- | C.5 | LC-MS: m/z = 476.12 |
| | created from editing field | methyl-1,3,4-thiadiazol- | and | [M+H]+, ESI pos |
| | codes. | 2- | D.1 | ¹H NMR (300 MHz, |
| | | yl)amino]benzimidazol- | and | DMSO-d6) δ = 10.09 (br |
| | | 1-yl]-3-(1-hydroxyethyl)- | E.13 | s, 1H), 9.09 (s, 1H), 8.77 |
| | | 2-pyridyl]-5-methyl- | | (d, J = 7.7 Hz, 1H), 8.46 |
| | | pyrazole-3-carbonitrile | | (d, J = 8.7 Hz, 1H), 8.30 |
| | | | | (d, J = 8.5 Hz, 1H), 7.97 |
| | | | | (d, J = 11.7 Hz, 1H), 7.11 |
| | | | | (d, J = 0.6 Hz, 1H), 5.54 |
| | | | | (d, J = 4.4 Hz, 1H), 4.57 |
| | | | | (dd, J = 4.5, 6.3 Hz, 1H), |
| | | | | 2.56 (s, 3H), 2.35 (s, 3H), |
| | | | | 1.26 (d, J = 6.4 Hz, 3H) |
| 388 | Error! Objects cannot be | 1-[6-[6- | C.6 | LC-MS: m/z = 518.2 |
| | created from editing field | (difluoromethoxy)-5-[(6- | and | [M+H]+, ESI pos |
| | codes. | methylpyridazin-3- | D.1 | |
| | | yl)amino]benzimidazol- | and | |
| | | 1-yl]-3-(1-hydroxyethyl)- | E.1 | |
| | | 2-pyridyl]-5-methyl- | | |
| | | pyrazole-3-carbonitrile | | |
| 389 | Error! Objects cannot be | 1-[6-[6-fluoro-5-[(5- | C.5 | LC-MS: m/z = 518.2 |
| | created from editing field | fluoro-6-methyl-3- | and | [M+H]+, ESI pos |
| | codes. | pyridyl)amino] | D.1 | |
| | | benzimidazol-1-yl]-3-(1- | and | |
| | | hydroxyethyl)-2- | E.14 | |
| | | pyridyl]-5-methyl- | | |
| | | pyrazole-3-carbonitrile | | |
| 390 | Error! Objects cannot be | 1-[6-[5-[[5-(azetidin-1- | C.5 | LC-MS: m/z = 487.3 |
| | created from editing field | yl)pyridazin-3-yl]amino]- | and | [M+H]+, ESI pos |
| | codes. | 6-fluoro-benzimidazol-1- | D.3 | |
| | | yl]-2-[3- | and | |
| | | (difluoromethyl)-5- | E.15 | |
| | | methyl-pyrazol-1-yl]-3- | | |
| | | pyridyl]ethanol | | |
| 391 | Error! Objects cannot be | 1-[6-[6-fluoro-5-[(5- | C.5 | LC-MS: m/z = 487.3 |
| | created from editing field | fluoro-6-methyl-3- | and | [M+H]+, ESI pos |
| | codes. | pyridyl)amino]benzimidazol- | D.1 | |
| | | 1-yl]-3-[(1S)-1- | and | |
| | | hydroxyethyl]-2- | E.14 | |
| | | pyridyl]-5-methyl- | | |
| | | pyrazole-3-carbonitrile | | |
| 394 | Error! Objects cannot be | 1-[6-[5-(7,8-dihydro- | C.7 | LC-MS: m/z = 524.2 |
| | created from editing field | 5~{H}-pyrano[4,3- | and | [M+H]+, ESI pos |
| | codes. | c]pyridazin-3-ylamino)- | D.1 | ¹H NMR (400 MHz, |
| | | 6-methoxy-benzimidazol- | and | METHANOL-d4) δ = |
| | | 1-yl]-3-(1-hydroxyethyl)- | E.16 | 8.80 (s, 1H), 8.53 (s, 1H), |
| | | 2-pyridyl]-5-methyl- | | 8.50 (d, J = 8.5 Hz, 1H), |
| | | pyrazole-3-carbonitrile | | 8.15 (d, J = 8.5 Hz, 1H), |
| | | | | 7.84 (s, 1H), 6.96 (s, 1H), |
| | | | | 6.87 (s, 1H), 4.80 (q, J = |
| | | | | 6.4 Hz, 1H), 4.73 (s, 2H), |
| | | | | 4.07 (t, J = 5.8 Hz, 2H), |
| | | | | 3.89 (s, 3H), 3.04 (t, J = |
| | | | | 5.8 Hz, 2H), 2.44 (s, 3H), |
| | | | | 1.42 (d, J = 6.5 Hz, 3H) |
| 400 | Error! Objects cannot be | (1S)-1-[2-[3- | C.7 | LC-MS: m/z = 523.2 |
| | created from editing field | (difluoromethoxy)-5- | and | [M+H]+, ESI pos |
| | codes. | methyl-pyrazol-1-yl]-6- | D.4 | ¹H NMR (400 MHz, |
| | | [6-methoxy-5-[(2- | and | METHANOL-d4) δ = |
| | | methylpyrimidin-5- | E.17 | 8.80 (br d, J = 5.5 Hz, |
| | | yl)amino]benzimidazol- | | 1H), 8.44-8.33 (m, 3H), |
| | | 1-yl]-3-pyridyl]ethanol | | 8.12 (d, J = 5.6 Hz, 1H), |
| | | | | 8.03-7.95 (m, 1H), 7.33 |
| | | | | (br d, J = 6.0 Hz, 1H), |
| | | | | 7.26-6.84 (m, 1H), 5.95 |
| | | | | (s, 1H), 4.61 (br s, 1H), |
| | | | | 4.01-3.92 (m, 3H), 2.64- |
| | | | | 2.57 (m, 3H), 2.24 (br s, |
| | | | | 3H), 1.38 (br d, J = 6.2 |
| | | | | Hz, 3H) |
| 402 | Error! Objects cannot be | 1-[6-[6- | C.6 | LC-MS: m/z = 518.2 |
| | created from editing field | (difluoromethoxy)-5-[(6- | and | [M+H]+, ESI pos |
| | codes. | methylpyridazin-3- | D.1 | |
| | | yl)amino]benzimidazol- | and | |
| | | 1-yl]-3-[(1S)-1- | E.1 | |
| | | hydroxyethyl]-2- | | |
| | | pyridyl]-5-methyl- | | |
| | | pyrazole-3-carbonitrile | | |
| 403 | Error! Objects cannot be | 1-[6-[6- | C.6 | LC-MS: m/z = 518.3 |
| | created from editing field | (difluoromethoxy)-5-[(6- | and | [M+H]+, ESI pos |
| | codes. | methylpyridazin-3- | D.1 | |
| | | yl)amino]benzimidazol- | and | |
| | | 1-yl]-3-[(1R)-1- | E.1 | |
| | | hydroxyethyl]-2- | | |
| | | pyridyl]-5-methyl- | | |
| | | pyrazole-3-carbonitrile | | |

### Examples 287, 290, 298, 299, 303, 305, 306, 309, 320-326, 329, 331, 344, 359, 360, 393

In analogy to Example 248, Examples in the following table were prepared, using the respective building blocks F.X in step 2.

### Building block F.1: 3,5-bis(difluoromethyl)-1H-pyrazole

Error! Objects cannot be created from editing field codes.

### CAS: 77614-79-0

### Building block F.2: 3-(difluoromethyl)pyrrolidine

Error! Objects cannot be created from editing field codes.

### CAS: 1376435-67-4

### Building block F.3: 1,1-difluoro-5-azaspiro[2.4]heptane

Error! Objects cannot be created from editing field codes.

### CAS: 1215166-77-0

### Building block F.4: 5-(difluoromethyl)-1H-pyrazole-3-carbonitrile

Error! Objects cannot be created from editing field codes.

### Step 1: methyl 5-(difluoromethyl)-1-(2-trimethylsilylethoxymethyl)pyrazole-3-carboxylate

To a solution of methyl 5-(difluoromethyl)-1H-pyrazole-3-carboxylate (1808112-13-1) (580 mg, 3.29 mmol, 1.0 eq) in DMF (6 mL) was added NaH (198 mg, 4.94 mmol, 1.5 eq) at 0° C. under N₂ and the mixture was stirred at 0° C. for 15 minutes. 2-(chloromethoxy)ethyl-trimethylsilane (824 mg, 4.94 mmol, 1.5 eq) was added and the mixture was stirred at 25° C. for 16 hours under N₂. The mixture was poured into water (30 mL) and extracted with EtOAc (2×20 mL). The combined organic layers were washed by brine (20 mL), dried over Na₂SO₄, filtered and concentrated. The residue was purified by flash chromatography (SiO₂, 0-10% EtOAc in PE) to methyl 5-(difluoromethyl)-1-(2-trimethylsilylethoxymethyl)pyrazole-3-carboxylate (670 mg, 2.19 mmol, 66% yield) as an off-white gum. LC-MS: m/z=249.3 [M+H]⁺, ESI pos.

### Step 2: 5-(difluoromethyl)-1-(2-trimethylsilylethoxymethyl)pyrazole-3-carboxamide

A solution of methyl 5-(difluoromethyl)-1-(2-trimethylsilylethoxymethyl)pyrazole-3-carboxylate (670 mg, 2.19 mmol, 1.0 eq) in ammonia in MeOH (7N) (11 mL) was stirred at 80° C. for 24 hours in a sealed tube. The mixture was concentrated to give 5-(difluoromethyl)-1-(2-trimethylsilylethoxymethyl)pyrazole-3-carboxamide (580 mg, 1.99 mmol, 91% yield) as colorless gum which was used without further purification. LC-MS: m/z=234.1 [M+H]⁺, ESI pos.

### Step 3: 5-(difluoromethyl)-1-(2-trimethylsilylethoxymethyl)pyrazole-3-carbonitrile

To a solution of 5-(difluoromethyl)-1-(2-trimethylsilylethoxymethyl)pyrazole-3-carboxamide (200 mg, 0.69 mmol, 1.0 eq) in DCM (4 mL) was added Burgess reagent (491 mg, 2.06 mmol, 3.0 eq). The mixture was stirred at 30° C. for 16 hours under N₂. The mixture was concentrated and the residue was purified by flash chromatography (SiO₂, 100% PE) to give 5-(difluoromethyl)-1-(2-trimethylsilylethoxymethyl)pyrazole-3-carbonitrile (50 mg, 0.18 mmol, 27% yield) as colorless oil. ¹H NMR (400 MHz, CDCl₃) δ=7.09 (s, 1H), 6.72 (t, J=54.5 Hz, 1H), 5.61 (s, 2H), 3.64 (t, J=8.2 Hz, 2H), 0.95 (t, J=8.2 Hz, 2H), 0.00 (s, 9H).

### Step 4: 5-(difluoromethyl)-1H-pyrazole-3-carbonitrile

A solution of 5-(difluoromethyl)-1-(2-trimethylsilylethoxymethyl)pyrazole-3-carbonitrile (100 mg, 0.37 mmol, 1.0 eq) in TFA (3.0 mL) was stirred at 30° C. for 1 hour. The mixture was diluted with EtOAc (5 mL) and concentrated in vacuo to give 5-(difluoromethyl)-1H-pyrazole-3-carbonitrile; 2,2,2-trifluoroacetic acid (90 mg, 0.35 mmol, 96% yield) as brown gum which was used without further purification.

### Building block E5: tert-butyl 3-cyano-7,7-difluoro-4,6-dihydro-1H-pyrazolo[4,3-c]pyridine-5-carboxylate

Error! Objects cannot be created from editing field codes.

### Step 1: tert-butyl 3,3-difluoro-4-pyrrolidin-1-yl-2,6-dihydropyridine-1-carboxylate

A mixture of tert-butyl 3,3-difluoro-4,4-dihydroxy-piperidine-1-carboxylate (5.0 g, 19.74 mmol, 1.0 eq) and pyrrolidine (1.63 mL, 19.76 mmol, 1.0 eq) in toluene (65 mL) was refluxed overnight with azeotropic removal of H₂O via Dean Stark trap. The reaction mixture was cooled to RT and concentrated to give tert-butyl 3,3-difluoro-4-pyrrolidin-1-yl-2,6-dihydropyridine-1-carboxylate (5.7 g, 19.77 mmol, 100% yield) as yellow solid which was used without further purification. ¹H NMR (400 MHz, CDCl₃) δ=4.47 (br s, 1H), 3.97 (br d, J=3.5 Hz, 2H), 3.77 (br t, J=11.5 Hz, 2H), 3.04 (br t, J=6.1 Hz, 4H), 1.80 (td, J=3.3, 6.5 Hz, 4H), 1.40 (s, 9H).

### Step 2: tert-butyl 5-(2-ethoxy-2-oxo-acetyl)-3,3-difluoro-4-pyrrolidin-1-yl-2,6-dihydropyridine-]-carboxylate

A solution of tert-butyl 3,3-difluoro-4-pyrrolidin-1-yl-2,6-dihydropyridine-1-carboxylate (5.7 g, 19.77 mmol, 1.0 eq) in DCM (250 mL) was bubbled with N₂ for 10 min. The solution was cooled to 0° C. Ethyl oxalyl chloride (3.25 g, 23.8 mmol, 1.2 eq) was added dropwise over 15 minutes while keeping the reaction temperature between 0° C. and 5° C. TEA (3.93 mL, 28.16 mmol, 1.42 eq) was added dropwise over 5 min. The reaction mixture was stirred at RT for 16 hours. The reaction mixture was diluted with H₂O (300 ml) and extracted with DCM (3×100 mL). The combined organic layers were dried over Na₂SO₄, filtered and concentrated. The residue was purified by flash chromatography (SiO₂, 0-25% EtOAc in PE) to give tert-butyl 5-(2-ethoxy-2-oxo-acetyl)-3,3-difluoro-4-pyrrolidin-1-yl-2,6-dihydropyridine-1-carboxylate (5.6 g, 14.42 mmol, 73% yield) as yellow oil. LC-MS: m/z=389.1 [M+H]⁺, ESI pos. ¹H NMR (400 MHz, CDCl₃) δ=4.40 (br s, 2H), 4.29 (q, J=7.2 Hz, 2H), 3.89 (t, J=12.2 Hz, 2H), 3.45 (br s, 4H), 1.99-1.94 (m, 4H), 1.47 (s, 9H), 1.35 (t, J=7.2 Hz, 3H)

### Step 3: 05-tert-butyl 03-ethyl 7,7-difluoro-4,6-dihydro-1H-pyrazolo[4,3-c]pyridine-3,5-dicarboxylate

Tert-butyl 5-(2-ethoxy-2-oxo-acetyl)-3,3-difluoro-4-pyrrolidin-1-yl-2,6-dihydropyridine-1-carboxylate (1.00 g, 2.57 mmol, 1.0 eq) was dissolved in EtOH (20 mL) and cooled to 0° C. Hydrazine hydrate (140 mg, 2.8 mmol, 1.09 eq) was added dropwise over 5 minutes. The reaction vessel was sealed and the mixture was stirred at 80° C. for 16 hours. The reaction mixture was diluted with H₂O (100 ml) and extracted with EtOAc (3×50 mL). The combined organic layers were dried over Na₂SO₄, filtered and concentrated. The residue was purified by flash chromatography (SiO₂, 0-50% EtOAc in PE) to give 05-tert-butyl 03-ethyl 7,7-difluoro-4,6-dihydro-1H-pyrazolo[4,3-c]pyridine-3,5-dicarboxylate (270 mg, 0.81 mmol, 32% yield) as a white solid. LC-MS: m/z=332.0 [M+H]⁺, ESI pos.

### Step 4: 05-tert-butyl 03-ethyl 7,7-difluoro-1-(2-trimethylsilylethoxymethyl)-4,6-dihydropyrazolo[4,3-c]pyridine-3,5-dicarboxylate

To a solution of 05-tert-butyl 03-ethyl 7,7-difluoro-4,6-dihydro-1H-pyrazolo[4,3-c]pyridine-3,5-dicarboxylate (260 mg, 0.78 mmol, 1.0 eq) in DMF (6 mL) was added NaH (38 mg, 0.95 mmol, 1.21 eq) at 0° C. The mixture was stirred at 0° C. for 30 minutes. 2-(trimethylsilyl)ethoxymethyl chloride (0.15 mL, 0.86 mmol, 1.1 eq) was added dropwise a and the mixture was stirred at 0° C. for 4 hours. The reaction mixture was diluted with sat. aq. NH₄Cl (50 mL) and extracted with EtOAc (3×30 mL). The combined organic layers were dried over Na₂SO₄, filtered and concentrated. The residue was purified by flash chromatography (SiO₂, 0-20% EtOAc in PE) to give 05-tert-butyl 03-ethyl 7,7-difluoro-1-(2-trimethylsilylethoxymethyl)-4,6-dihydropyrazolo[4,3-c]pyridine-3,5-dicarboxylate (270 mg, 0.58 mmol, 75% yield) as colorless oil.

### Step 5: tert-butyl 3-carbamoyl-7,7-difluoro-1-(2-trimethylsilylethoxymethyl)-4,6-dihydropyrazolo[4,3-c]pyridine-5-carboxylate

A solution of 05-tert-butyl 03-ethyl 7,7-difluoro-1-(2-trimethylsilylethoxymethyl)-4,6-dihydropyrazolo[4,3-c]pyridine-3,5-dicarboxylate (270 mg, 0.58 mmol, 1.0 eq) in ammonia in MeOH (7N) (6.0 mL). The reaction mixture was stirred at 80° C. for 60 hours. The reaction mixture was concentrated to give tert-butyl 3-carbamoyl-7,7-difluoro-1-(2-trimethylsilylethoxymethyl)-4,6-dihydropyrazolo[4,3-c]pyridine-5-carboxylate (220 mg, 0.51 mmol, 87% yield) as a white solid.

### Step 6: tert-butyl 3-cyano-7,7-difluoro-1-(2-trimethylsilylethoxymethyl)-4,6-dihydropyrazolo[4,3-c]pyridine-5-carboxylate

To a solution of tert-butyl 3-carbamoyl-7,7-difluoro-1-(2-trimethylsilylethoxymethyl)-4,6-dihydropyrazolo[4,3-c]pyridine-5-carboxylate (190 mg, 0.44 mmol, 1.0 eq) in pyridine (5.0 mL) was added trifluoroacetic anhydride (0.26 mL, 1.81 mmol, 4.1 eq) dropwise at 0° C. under N₂. The mixture was stirred at 0° C. for 1 hour. The mixture was concentracted and the residue was purified by flash chromatography (SiO₂, 0-10% EtOAc in PE) to give tert-butyl 3-cyano-7,7-difluoro-1-(2-trimethylsilylethoxymethyl)-4,6-dihydropyrazolo[4,3-c]pyridine-5-carboxylate (170 mg, 0.41 mmol, 93% yield) as light yellow oil. LC-MS: m/z=415.8 [M+H]⁺, ESI pos.

### Step 7: tert-butyl 3-cyano-7,7-difluoro-4,6-dihydro-1H-pyrazolo[4,3-c]pyridine-5-carboxylate

To a solution of tert-butyl 3-cyano-7,7-difluoro-1-(2-trimethylsilylethoxymethyl)-4,6-dihydropyrazolo[4,3-c]pyridine-5-carboxylate (70 mg, 0.17 mmol, 1.0 eq) in THF (2 mL) was added TBAF (1M in THF) (0.84 mL, 0.84 mmol, 5.0 eq) and ethylenediamine (51 mg, 0.85 mmol, 5.0 eq) The mixture was stirred at 60° C. for 3 hours under N₂. The reaction mixture was cooled to RT and diluted with saturated aqueous NH₄Cl (30 ml) and extracted with ethyl acetate (3×15 mL). The combined organic layers were dried over Na₂SO₄, filtered and concentrated. The residue was purified by preparative TLC (SiO₂, PE: EtOAc 5:1) to give tert-butyl 3-cyano-7,7-difluoro-4,6-dihydro-1H-pyrazolo[4,3-c]pyridine-5-carboxylate (25 mg, 0.09 mmol, 52% yield) as colorless oil. ¹H NMR (400 MHz, CDCl₃) δ=4.62 (br s, 2H), 4.00 (br s, 2H), 1.49 (s, 9H).

### Building block F6: 2-(trifluoromethyl)morpholine

Error! Objects cannot be created from editing field codes.

### CAS: 1196532-95-2

### Building block F.7: 3-azabicyclo[3.1.O]hexane-1-carbonitrile

Error! Objects cannot be created from editing field codes.

### CAS: 1422005-81-9

### Building block F8: 5,5-Difluoro-2-azabicyclo[2.2.]heptane

Error! Objects cannot be created from editing field codes.

### CAS: 1214875-10-1

### Building block F9: 2-(difluoromethyl)morpholine

Error! Objects cannot be created from editing field codes.

### CAS: 1242465-34-4

### Building block F.10: 6,6-difluoro-2-azabicyclo[2.2.I]heptane

Error! Objects cannot be created from editing field codes.

### CAS: 1357352-59-0

### Building block F.11: 7-(difluoromethyl)-5-azaspiro[2.4]heptane

Error! Objects cannot be created from editing field codes.

### CAS: 2137834-10-5

### Building block F.12: 6,6-difluoro-3-azabicyclo[3.2.O]heptane

Error! Objects cannot be created from editing field codes.

### CAS: 1214875-41-8

### Building block F.13: 1,1-dimethylethyl 4,7-diazaspiro[2.5]octane-4-carboxylate

Error! Objects cannot be created from editing field codes.

### CAS: 674792-08-6

### Building block F.14: 1,1-dimethylethyl 2-(trifluoromethyl)-1-piperazinecarboxylate

Error! Objects cannot be created from editing field codes.

### CAS: 886779-77-7

### Building block F.15: 5-(difluoromethyl)-3-methyl-1H-1,2,4-triazole

Error! Objects cannot be created from editing field codes.

### CAS: 1094760-03-8

### Building block F.16: 6-azaspiro[3.4]octane-8-carbonitrile

Error! Objects cannot be created from editing field codes.

### CAS: 2092409-34-0

### Building block F.17: 2-morpholinecarbonitrile

Error! Objects cannot be created from editing field codes.

### CAS: 135782-24-0

### Building block F.18: 1,1-dimethylethyl 3,3-difluoro-4-hydroxy-1-pyrrolidinecarboxylate

Error! Objects cannot be created from editing field codes.

### CAS: 1434141-81-7

### Building block F.19: 3-(difluoromethoxy)-5-methyl-1H-pyrazole

Error! Objects cannot be created from editing field codes.

### CAS: 2108002-48-6

### Building block F.20: 4,5-Dimethyl-1H-pyrazole-3-carbonitrile

Error! Objects cannot be created from editing field codes.

### CAS: 2138948-90-8

| | | | Building | |
|---|---|---|---|---|
| Ex. | Structure | Systematic name | blocks | Analytics |
| 287 | Error! Objects cannot be | (1S)-1-[2-[3,5- | F.1 | LC-MS: m/z = 513.3 |
| | created from editing field | bis(difluoromethyl)pyrazol- | | [M + H]+, ESI pos |
| | codes. | 1-yl]-6-[5-[(6- | | |
| | | methylpyridazin-3- | | |
| | | yl)amino]benzimidazol- | | |
| | | 1-yl]-3-pyridyl]ethanol | | |
| 288 | 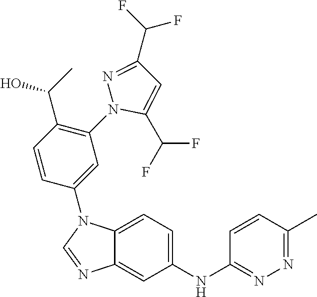 [SMILES: Cc1ccc(Nc2ccc3c(c2)ncn3-c2ccc([C@@H](C)O)c(-n3nc(C(F)F)cc3C(F)F)c2)nn1] | (1R)-1-[2-[3,5- bis(difluoromethyl)pyra zol-1-yl]-6-[5-[(6- methylpyridazin-3- yl)amino]benzimidazol- 1-yl]-3-pyridyl]ethanol | F.1 | LC-MS: m/z = 513.3 [M + H]+, ESI pos |
| 290 | Error! Objects cannot be | 1-[2-[3- | F.19 | LC-MS: m/z = 493.3 |
| | created from editing field | (difluoromethoxy)-5- | | [M + H]+, ESI pos |
| | codes. | methyl-pyrazol-1-yl]-6- | | ¹H NMR (400 MHz, |
| | | [5-[(6-methylpyridazin- | | METHANOL-d4) δ = |
| | | 3- | | 8.90 (s, 1H), 8.47 (d, J = |
| | | yl)amino]benzimidazol- | | 8.3 Hz, 1H), 8.23-8.20 |
| | | 1-yl]-3-pyridyl]ethanol | | (m, 1H), 8.17 (d, J = 9.2 Hz, |
| | | | | 1H), 8.06 (d, J = 8.3 Hz, |
| | | | | 1H), 7.62 (br d, J = |
| | | | | 9.2 Hz, 1H), 7.40 (d, J = |
| | | | | 9.2 Hz, 1H), 7.26-6.90 |
| | | | | (d, 1H), 7.18 (d, J = 9.4 Hz, |
| | | | | 1H), 6.09 (s, 1H), |
| | | | | 5.00 (br d, J = 2.3 Hz, |
| | | | | 1H), 2.56 (s, 3H), 2.40 |
| | | | | (s, 3H), 1.44 (d, J = 6.4 Hz, |
| | | | | 3H) |
| 298 | Error! Objects cannot be | 1-[2-[3- | F.2 | LC-MS: m/z = 466.39 |
| | created from editing field | (difluoromethyl)pyrrolidin- | | [M + H]+, ESI pos |
| | codes. | 1-yl]-6-[5-[(6- | | ¹H NMR (300 MHz, |
| | | methylpyridazin-3- | | DMSO-d6) δ = 9.16 (s, |
| | | yl)amino]benzimidazol- | | 1H), 8.83 (s, 1H), 8.37- |
| | | 1-yl]-3-pyridyl]ethanol | | 8.28 (m, 1H), 8.17 (d, J = |
| | | | | 8.9 Hz, 1H), 7.99- |
| | | | | 7.92 (m, 1H), 7.55 (td, J = |
| | | | | 2.2, 8.9 Hz, 1H), 7.32 |
| | | | | (d, J = 9.1 Hz, 1H), |
| | | | | 7.26 (d, J = 8.1 Hz, 1H), |
| | | | | 7.08 (d, J = 9.1 Hz, 1H), |
| | | | | 6.44-5.98 (m, 1H), 5.41- |
| | | | | 5.15 (m, 1H), 5.07 (q, J = |
| | | | | 6.3 Hz, 1H), 3.81- |
| | | | | 3.60 (m, 4H), 2.93-2.76 |
| | | | | (m, 1H), 2.48 (br s, 3H), |
| | | | | 2.22-1.88 (m, 2H), 1.41 |
| | | | | (d, J = 6.2 Hz, 3H) |
| 299 | Error! Objects cannot be | 1-[2-(2,2-difluoro-5- | F.3 | LC-MS: m/z = 478.35 |
| | created from editing field | azaspiro[2.4]heptan-5- | | [M + H]+, ESI pos |
| | codes. | yl)-6-[5-[(6- | | ¹H NMR (300 MHz, |
| | | methylpyridazin-3- | | DMSO-d6) δ = 9.15 (s, |
| | | yl)amino]benzimidazol- | | 1H), 8.83 (s, 1H), 8.31 |
| | | 1-yl]-3-pyridyl]ethanol | | (d, J = 2.0 Hz, 1H), 8.14 |
| | | | | (d, J = 8.9 Hz, 1H), 7.98 |
| | | | | (d, J = 8.1 Hz, 1H), 7.55 |
| | | | | (dd, J = 2.0, 8.9 Hz, |
| | | | | 1H), 7.31 (t, J = 8.5 Hz, |
| | | | | 2H), 7.07 (d, J = 9.1 Hz, |
| | | | | 1H), 5.24 (br s, 1H), |
| | | | | 5.13-4.99 (m, 1H), 3.88- |
| | | | | 3.60 (m, 4H), 2.48 (s, |
| | | | | 3H), 2.25-1.99 (m, 2H), |
| | | | | 1.79-1.57 (m, 2H), 1.40 |
| | | | | (dd, J = 4.0, 6.2 Hz, 3H) |
| 303 | Error! Objects cannot be | 5-(difluoromethyl)-2-[3- | F.4 | LC-MS: m/z = 488.1 |
| | created from editing field | (1-hydroxyethyl)-6-[5- | | [M + H]+, ESI pos |
| | codes. | [(6-methylpyridazin-3- | | ¹H NMR (400 MHz, |
| | | yl)amino]benzimidazol- | | METHANOL-d4) δ = |
| | | 1-yl]-2- | | 8.88 (s, 1H), 8.55 (d, J = |
| | | pyridyl]pyrazole-3- | | 8.4 Hz, 1H), 8.23 (d, J = |
| | | carbonitrile | | 2.0 Hz, 1H), 8.16 (d, J = |
| | | | | 8.6 Hz, 1H), 8.06 (d, J = |
| | | | | 8.9 Hz, 1H), 7.59 (dd, J = |
| | | | | 2.1, 8.9 Hz, 1H), 7.46 |
| | | | | (s, 1H), 7.39 (d, J = 9.2 Hz, |
| | | | | 1H), 7.37-7.08 (m, |
| | | | | 2H), 5.00 (q, J = 6.4 Hz, |
| | | | | 1H), 2.54 (s, 3H), 1.44 |
| | | | | (d, J = 6.4 Hz, 3H). |
| 305 | Error! Objects cannot be | 7,7-difluoro-1-[3-(1- | F.5 | LC-MS: m/z = 529.3 |
| | created from editing field | hydroxyethyl)-6-[5-[(6- | followd by | [M + H]+, ESI pos |
| | codes. | methylpyridazin-3- | Boc | ¹H NMR (400 MHz, |
| | | yl)amino]benzimidazol- | deprotection | DMSO-d6) δ = 9.24 (s, |
| | | 1-yl]-2-pyridyl]-5,6- | in | 1H), 9.02 (s, 1H), 8.52 |
| | | dihydro-4~{H}- | analogy to | (d, J = 8.5 Hz, 1H), 8.46 |
| | | pyrazolo[4,3- | Example | (s, 1H), 8.36 (d, J = 1.8 Hz, |
| | | c]pyridine-3- | 205, step 8 | 1H), 8.29 (d, J = 8.5 Hz, |
| | | carbonitrile | | 1H), 8.21 (d, J = 8.9 Hz, |
| | | | | 1H), 7.60 (dd, J = |
| | | | | 2.0, 9.0 Hz, 1H), 7.34 |
| | | | | (d, J = 9.0 Hz, 1H), 7.09 |
| | | | | (d, J = 9.1 Hz, 1H), 5.68- |
| | | | | 5.50 (m, 1H), 5.06- |
| | | | | 4.88 (m, 1H), 4.05 (br s, |
| | | | | 2H), 3.43-3.41 (m, 2H), |
| | | | | 2.48 (br s, 3H), 1.38 (d, |
| | | | | J = 6.4 Hz, 3H). |
| 306 | Error! Objects cannot be | 5-(difluoromethyl)-1-[3- | F.4 | LC-MS: m/z = 488.1 |
| | created from editing field | (1-hydroxyethyl)-6-[5- | | [M + H]+, ESI pos |
| | codes. | [(6-methylpyridazin-3- | | ¹H NMR (400 MHz, |
| | | yl)amino]benzimidazol- | | DMSO-d6) δ = 9.28 (s, |
| | | 1-yl]-2- | | 1H), 9.02 (s, 1H), 8.52 |
| | | pyridyl]pyrazole-3- | | (d, J = 8.4 Hz, 1H), 8.44- |
| | | carbonitrile | | 8.40 (m, 1H), 8.29 (d, J = |
| | | | | 8.6 Hz, 1H), 8.19 (d, J = |
| | | | | 8.8 Hz, 1H), 7.95 (s, |
| | | | | 1H), 7.55 (dd, J = 2.0, |
| | | | | 8.9 Hz, 1H), 7.44-7.14 |
| | | | | (m, 2H), 7.09 (d, J = 9.0 Hz, |
| | | | | 1H), 5.82-5.32 (m, |
| | | | | 1H), 4.95 (q, J = 6.2 Hz, |
| | | | | 1H), 2.48 (s, 3H), 1.38 |
| | | | | (d, J = 6.4 Hz, 3H) |
| 309 | Error! Objects cannot be | 1-[3-(1-hydroxyethyl)- | F.20 | LC-MS: m/z = 466.1 |
| | created from editing field | 6-[5-[(6- | | [M + H]+, ESI pos |
| | codes. | methylpyridazin-3- | | ¹H NMR (400 MHz, |
| | | yl)amino]benzimidazol- | | METHANOL-d4) δ = |
| | | 1-yl]-2-pyridyl]-4,5- | | 8.92 (s, 1H), 8.51 (d, J = |
| | | dimethyl-pyrazole-3- | | 8.6 Hz, 1H), 8.31 (br s, |
| | | carbonitrile | | 1H), 8.25 (d, J = 2.0 Hz, |
| | | | | 1H), 8.14 (t, J = 8.9 Hz, |
| | | | | 2H), 7.61 (dd, J = 2.1, |
| | | | | 8.9 Hz, 1H), 7.39 (d, J = |
| | | | | 9.2 Hz, 1H), 7.16 (d, J = |
| | | | | 9.0 Hz, 1H), 4.78 (q, J = |
| | | | | 6.4 Hz, 1H), 2.55 (s, |
| | | | | 3H), 2.34 (s, 3H), 2.28 |
| | | | | (s, 3H), 1.43 (d, J = 6.5 Hz, |
| | | | | 3H) |
| 320 | Error! Objects cannot be | 1-[6-[5-[(6- | F.6 | LC-MS: m/z = 500.38 |
| | created from editing field | methylpyridazin-3- | | [M + H]+, ESI pos |
| | codes. | yl)amino]benzimidazol- | | ¹H NMR (300 MHz, |
| | | 1-yl]-2-[2- | | DMSO-d6) δ = 9.18 (s, |
| | | (trifluoromethyl)morpholin- | | 1H), 8.89 (s, 1H), 8.39 |
| | | 4-yl]-3- | | (dd, J = 2.0, 4.0 Hz, |
| | | pyridyl]ethanol | | 1H), 8.17 (dd, J = 3.2, |
| | | | | 8.9 Hz, 1H), 8.08 (d, J = |
| | | | | 8.3 Hz, 1H), 7.64 (d, J = |
| | | | | 8.1 Hz, 1H), 7.56-7.48 |
| | | | | (m, 1H), 7.33 (d, J = 9.3 Hz, |
| | | | | 1H), 7.08 (dd, J = |
| | | | | 1.0, 9.1 Hz, 1H), 5.44- |
| | | | | 5.19 (m, 1H), 5.02 (q, J = |
| | | | | 6.3 Hz, 1H), 4.67- |
| | | | | 4.33 (m, 1H), 4.18-4.03 |
| | | | | (m, 1H), 3.99-3.72 (m, |
| | | | | 2H), 3.68-3.51 (m, 1H), |
| | | | | 3.23-3.13 (m, 1H), 3.13- |
| | | | | 2.97 (m, 1H), 2.48 (br s, |
| | | | | 3H), 1.44 (d, J = 6.4 Hz, |
| | | | | 3H) |
| 321 | Error! Objects cannot be | 3-[3-(1-hydroxyethyl)- | F.7 | LC-MS: m/z = 453.41 |
| | created from editing field | 6-[5-[(6- | | [M + H]+, ESI pos |
| | codes. | methylpyridazin-3- | | ¹H NMR (300 MHz, |
| | | yl)amino]benzimidazol- | | DMSO-d6) δ = 9.17 (s, |
| | | 1-yl]-2-pyridyl]-3- | | 1H), 8.84 (s, 1H), 8.33 |
| | | azabicyclo[3.1.0]hexane- | | (d, J = 2.0 Hz, 1H), 8.10 |
| | | 1-carbonitrile | | (d, J = 8.9 Hz, 1H), 7.99 |
| | | | | (dd, J = 1.6, 8.1 Hz, |
| | | | | 1H), 7.57 (dd, J = 2.0, |
| | | | | 8.9 Hz, 1H), 7.38 (d, J = |
| | | | | 8.1 Hz, 1H), 7.33 (d, J = |
| | | | | 9.1 Hz, 1H), 7.08 (d, J = |
| | | | | 9.1 Hz, 1H), 5.38-5.20 |
| | | | | (m, 1H), 4.93 (q, J = 6.0 Hz, |
| | | | | 1H), 4.13 (dd, J = |
| | | | | 10.0, 18.6 Hz, 1H), 3.90 |
| | | | | (dd, J = 10.5, 19.1 Hz, |
| | | | | 1H), 3.79-3.55 (m, 2H), |
| | | | | 2.55-2.52 (m, 1H), 2.48 |
| | | | | (br s, 3H), 1.50-1.42 |
| | | | | (m, 1H), 1.38 (dd, J = |
| | | | | 3.8, 6.2 Hz, 3H), 1.26- |
| | | | | 1.16 (m, 1H) |
| 322 | Error! Objects cannot be | 1-[2-(5,5-difluoro-2- | F.8 | LC-MS: m/z = 478.39 |
| | created from editing field | azabicyclo[2.2.1]heptan- | | [M + H]+, ESI pos |
| | codes. | 2-yl)-6-[5-[(6- | | ¹H NMR (300 MHz, |
| | | methylpyridazin-3- | | DMSO-d6) δ = 9.16 (s, |
| | | yl)amino]benzimidazol- | | 1H), 8.86-8.79 (m, 1H), |
| | | 1-yl]-3-pyridyl]ethanol | | 8.29 (d, J = 2.0 Hz, 1H), |
| | | | | 8.06 (d, J = 8.9 Hz, 1H), |
| | | | | 8.01-7.89 (m, 1H), 7.62- |
| | | | | 7.52 (m, 1H), 7.33 (d, J = |
| | | | | 9.1 Hz, 1H), 7.28 (d, J = |
| | | | | 8.1 Hz, 1H), 7.08 (d, J = |
| | | | | 9.1 Hz, 1H), 5.38- |
| | | | | 5.06 (m, 1H), 5.01-4.91 |
| | | | | (m, 1H), 4.79-4.61 (m, |
| | | | | 1H), 3.89-3.71 (m, 1H), |
| | | | | 3.59-3.42 (m, 1H), 3.03- |
| | | | | 2.93 (m, 1H), 2.48 (s, |
| | | | | 3H), 2.42-2.23 (m, 2H), |
| | | | | 1.97 (s, 2H), 1.52-1.28 |
| | | | | (m, 3H) |
| 323 | Error! Objects cannot be | 1-[2-[2- | F.9 | LC-MS: m/z = 482.41 |
| | created from editing field | (difluoromethyl)morpholin- | | [M + H]+, ESI pos |
| | codes. | 4-yl]-6-[5-[(6- | | ¹H NMR (300 MHz, |
| | | methylpyridazin-3- | | DMSO-d6) δ = 9.18 (s, |
| | | yl)amino]benzimidazol- | | 1H), 8.88 (s, 1H), 8.36 |
| | | 1-yl]-3-pyridyl]ethanol | | (s, 1H), 8.19 (dd, J = |
| | | | | 1.9, 8.8 Hz, 1H), 8.07 |
| | | | | (d, J = 8.3 Hz, 1H), 7.63 |
| | | | | (dd, J = 0.8, 8.1 Hz, |
| | | | | 1H), 7.54 (dd, J = 2.1, |
| | | | | 9.0 Hz, 1H), 7.33 (d, J = |
| | | | | 9.1 Hz, 1H), 7.08 (d, J = |
| | | | | 9.3 Hz, 1H), 6.41-5.94 |
| | | | | (m, 1H), 5.43-5.21 (m, |
| | | | | 1H), 5.02 (q, J = 6.6 Hz, |
| | | | | 1H), 4.16-3.93 (m, 2H), |
| | | | | 3.89-3.74 (m, 1H), 3.71- |
| | | | | 3.51 (m, 1H), 3.45- |
| | | | | 3.35 (m, 1H), 3.23-2.93 |
| | | | | (m, 2H), 2.48 (br s, 3H), |
| | | | | 1.44 (d, J = 6.2 Hz, 3H) |
| 324 | Error! Objects cannot be | 1-[2-(6,6-difluoro-2- | F.10 | LC-MS: m/z = 478.39 |
| | created from editing field | azabicyclo[2.2.1]heptan- | | [M + H]+, ESI pos |
| | codes. | 2-yl)-6-[5-[(6- | | ¹H NMR (300 MHz, |
| | | methylpyridazin-3- | | DMSO-d6) δ = 9.16 (s, |
| | | yl)amino]benzimidazol- | | 1H), 8.81 (s, 1H), 8.31 |
| | | 1-yl]-3-pyridyl]ethanol | | (d, J = 1.6 Hz, 1H), 8.16- |
| | | | | 8.06 (m, 1H), 8.02- |
| | | | | 7.89 (m, 1H), 7.61-7.51 |
| | | | | (m, 1H), 7.33 (d, J = 9.1 Hz, |
| | | | | 1H), 7.26-7.16 (m, |
| | | | | 1H), 7.08 (d, J = 9.1 Hz, |
| | | | | 1H), 5.43-5.18 (m, 1H), |
| | | | | 5.17-5.02 (m, 1H), 5.01- |
| | | | | 4.85 (m, 1H), 3.86- |
| | | | | 3.59 (m, 1H), 3.55-3.40 |
| | | | | (m, 1H), 2.85-2.76 (m, |
| | | | | 1H), 2.48 (s, 3H), 2.24- |
| | | | | 1.92 (m, 2H), 1.84 (br s, |
| | | | | 2H), 1.40 (d, J = 6.2 Hz, |
| | | | | 3H) |
| 325 | Error! Objects cannot be | 1-[2-[7- | F.11 | LC-MS: m/z = 492.35 |
| | created from editing field | (difluoromethyl)-5- | | [M + H]+, ESI pos |
| | codes. | azaspiro[2.4]heptan-5- | | ¹H NMR (300 MHz, |
| | | yl]-6-[5-[(6- | | DMSO-d6) δ = 9.15 (s, |
| | | methylpyridazin-3- | | 1H), 9.02-8.78 (m, 1H), |
| | | yl)amino]benzimidazol- | | 8.28 (dd, J = 2.1, 3.3 Hz, |
| | | 1-yl]-3-pyridyl]ethanol | | 1H), 8.14 (dd, J = 3.6, |
| | | | | 8.9 Hz, 1H), 8.02-7.90 |
| | | | | (m, 1H), 7.56 (td, J = |
| | | | | 2.4, 8.9 Hz, 1H), 7.38- |
| | | | | 7.29 (m, 1H), 7.24 (dd, J = |
| | | | | 4.2, 8.1 Hz, 1H), 7.07 |
| | | | | (d, J = 9.3 Hz, 1H), 6.45- |
| | | | | 5.98 (m, 1H), 5.39- |
| | | | | 5.14 (m, 1H), 5.12-5.01 |
| | | | | (m, 1H), 4.14-3.99 (m, |
| | | | | 1H), 3.92-3.76 (m, 2H), |
| | | | | 3.42-3.34 (m, 1H), 3.30- |
| | | | | 3.16 (m, 1H), 2.48 (s, |
| | | | | 3H), 1.49-1.29 (m, 3H), |
| | | | | 1.10-0.94 (m, 1H), 0.71 |
| | | | | (br s, 3H) |
| 326 | Error! Objects cannot be | 1-[2-(6,6-difluoro-3- | F.12 | LC-MS: m/z = 478.32 |
| | created from editing field | azabicyclo[3.2.0]heptan- | | [M + H]+, ESI pos |
| | codes. | 3-yl)-6-[5-[(6- | | ¹H NMR (300 MHz, |
| | | methylpyridazin-3- | | DMSO-d6) δ = 9.16 (s, |
| | | yl)amino]benzimidazol- | | 1H), 8.85 (s, 1H), 8.30 |
| | | 1-yl]-3-pyridyl]ethanol | | (d, J = 2.0 Hz, 1H), 8.18 |
| | | | | (d, J = 8.9 Hz, 1H), 8.04 |
| | | | | (d, J = 8.3 Hz, 1H), 7.56 |
| | | | | (dd, J = 2.1, 9.0 Hz, |
| | | | | 1H), 7.51-7.46 (m, 1H), |
| | | | | 7.33 (d, J = 9.1 Hz, 1H), |
| | | | | 7.08 (d, J = 9.1 Hz, 1H), |
| | | | | 5.45-5.21 (m, 1H), 5.19- |
| | | | | 5.00 (m, 1H), 4.00 (d, J = |
| | | | | 11.3 Hz, 1H), 3.60 (d, |
| | | | | J = 10.5 Hz, 1H), 3.48- |
| | | | | 3.34 (m, 2H), 3.25-3.12 |
| | | | | (m, 1H), 2.99-2.54 (m, |
| | | | | 3H), 2.48 (s, 3H), 1.50- |
| | | | | 1.39 (m, 3H) |
| 329 | Error! Objects cannot be | 1-[2-(4,7- | F.13 | LC-MS: m/z = 457.32 |
| | created from editing field | diazaspiro[2.5]octan-7- | followd by | [M + H]+, ESI pos |
| | codes. | yl)-6-[5-[(6- | Boc | ¹H NMR (300 MHz, |
| | | methylpyridazin-3- | deprotection | DMSO-d6) δ = 9.17 (s, |
| | | yl)amino]benzimidazol- | in | 1H), 8.86 (s, 1H), 8.30 |
| | | 1-yl]-3-pyridyl]ethanol | analogy to | (d, J = 1.8 Hz, 1H), 8.23 |
| | | | Example | (d, J = 8.9 Hz, 1H), 8.00 |
| | | | 205, step 8 | (d, J = 8.1 Hz, 1H), 7.61- |
| | | | | 7.53 (m, 2H), 7.33 (d, J = |
| | | | | 9.1 Hz, 1H), 7.08 (d, J = |
| | | | | 9.1 Hz, 1H), 5.23 (d, J = |
| | | | | 4.6 Hz, 1H), 5.10- |
| | | | | 4.97 (m, 1H), 3.29-3.23 |
| | | | | (m, 1H), 3.19-3.05 (m, |
| | | | | 3H), 3.02-2.89 (m, 3H), |
| | | | | 2.48-2.47 (m, 3H), 1.41 |
| | | | | (d, J = 6.2 Hz, 3H), 0.54 |
| | | | | (s, 4H) |
| 331 | Error! Objects cannot be | 1-[6-[5-[(6- | F.14 | LC-MS: m/z = 499.58 |
| | created from editing field | methylpyridazin-3- | | [M + H]+, ESI pos |
| | codes. | yl)amino]benzimidazol- | | ¹H NMR (300 MHz, |
| | | 1-yl]-2-[3- | | DMSO-d6) δ = 9.24 (d, |
| | | (trifluoromethyl)piperazin- | | J = 16.1 Hz, 1H), 8.87 |
| | | 1-yl]-3- | | (d, J = 3.6 Hz, 1H), 8.46- |
| | | pyridyl]ethanol | | 8.33 (m, 1H), 8.25- |
| | | | | 8.14 (m, 1H), 8.05 (dd, J = |
| | | | | 2.7, 8.2 Hz, 1H), 7.65- |
| | | | | 7.57 (m, 1H), 7.56- |
| | | | | 7.45 (m, 1H), 7.32 (dd, J = |
| | | | | 2.7, 9.2 Hz, 1H), 7.16- |
| | | | | 7.05 (m, 1H), 5.45- |
| | | | | 5.15 (m, 1H), 5.11-4.94 |
| | | | | (m, 1H), 4.42-4.25 (m, |
| | | | | 1H), 3.79-3.37 (m, 3H), |
| | | | | 3.16-2.85 (m, 4H), 2.48 |
| | | | | (br s, 3H), 1.53-1.33 |
| | | | | (m, 3H) |
| 344 | Error! Objects cannot be | 1-[2-[3- | F.15 | LC-MS: m/z = 478.34 |
| | created from editing field | (difluoromethyl)-5- | | [M + H]+, ESI pos |
| | codes. | methyl-1,2,4-triazol-1- | | ¹H NMR (300 MHz, |
| | | yl]-6-[5-[(6- | | DMSO-d6) δ = 9.22 (s, |
| | | methylpyridazin-3- | | 1H), 9.03 (s, 1H), 8.51- |
| | | yl)amino]benzimidazol- | | 8.43 (m, 2H), 8.28 (d, J = |
| | | 1-yl]-3-pyridyl]ethanol | | 8.5 Hz, 1H), 8.07 (d, J = |
| | | | | 8.9 Hz, 1H), 7.49 (dd, |
| | | | | J = 2.2, 8.9 Hz, 1H), |
| | | | | 7.33 (d, J = 9.1 Hz, 1H), |
| | | | | 7.09 (d, J = 9.3 Hz, 1H), |
| | | | | 7.19 (br t, J = 52.9 Hz, |
| | | | | 1H), 5.69-5.44 (m, 1H), |
| | | | | 4.69 (q, J = 6.2 Hz, 1H), |
| | | | | 2.55 (s, 3H), 2.48 (br s, |
| | | | | 3H), 1.30 (d, J = 6.4 Hz, |
| | | | | 3H) |
| 359 | Error! Objects cannot be | 6-[3-(1-hydroxyethyl)- | F.16 | LC-MS: m/z = 481.32 |
| | created from editing field | 6-[5-[(6- | | [M + H]+, ESI pos |
| | codes. | methylpyridazin-3- | | ¹H NMR (300 MHz, |
| | | yl)amino]benzimidazol- | | DMSO-d6) δ = 9.15 (s, |
| | | 1-yl]-2-pyridyl]-6- | | 1H), 8.84 (s, 1H), 8.12 |
| | | azaspiro[3.4]octane-8- | | (dd, J = 0.9, 9.0 Hz, |
| | | carbonitrile | | 1H), 7.97 (dd, J = 5.6, |
| | | | | 8.1 Hz, 1H), 7.58 (td, J = |
| | | | | 1.7, 8.9 Hz, 1H), 7.33 |
| | | | | (d, J = 9.1 Hz, 1H), 7.28 |
| | | | | (d, J = 8.1 Hz, 1H), 7.08 |
| | | | | (d, J = 9.3 Hz, 1H), 5.26 |
| | | | | (dd, J = 4.7, 13.8 Hz, |
| | | | | 1H), 5.11-4.97 (m, 1H), |
| | | | | 4.01-3.51 (m, 5H), 2.32- |
| | | | | 1.81 (m, 7H), 1.39 (dd, |
| | | | | J = 6.2, 13.7 Hz, 3H) |
| 360 | Error! Objects cannot be | 4-[3-(1-hydroxyethyl)- | F.17 | LC-MS: m/z = 457.27 |
| | created from editing field | 6-[5-[(6- | | [M + H]+, ESI pos |
| | codes. | methylpyridazin-3- | | ¹H NMR (300 MHz, |
| | | yl)amino]benzimidazol- | | DMSO-d6) δ = 9.21- |
| | | 1-yl]-2- | | 9.14 (m, 1H), 8.89 (s, |
| | | pyridyl]morpholine-2- | | 1H), 8.35-8.30 (m, 1H), |
| | | carbonitrile | | 8.19 (dd, J = 1.3, 8.8 Hz, |
| | | | | 1H), 8.10 (dd, J = 2.2, |
| | | | | 8.3 Hz, 1H), 7.67 (d, J = |
| | | | | 8.3 Hz, 1H), 7.58 (td, J = |
| | | | | 1.7, 9.0 Hz, 1H), 7.33 |
| | | | | (d, J = 9.3 Hz, 1H), 7.08 |
| | | | | (d, J = 9.1 Hz, 1H), 5.33 |
| | | | | (d, J = 4.6 Hz, 1H), 5.25- |
| | | | | 5.19 (m, 1H), 5.16- |
| | | | | 5.09 (m, 1H), 4.07-3.94 |
| | | | | (m, 2H), 3.77-3.44 (m, |
| | | | | 2H), 3.31-3.24 (m, 2H), |
| | | | | 2.48 (br s, 3H), 1.45 (dd, |
| | | | | J = 2.2, 6.2 Hz, 3H) |
| 393 | Error! Objects cannot be | 1-[2-(4,4- | F.18 | LC-MS: m/z = 468.09 |
| | created from editing field | difluoropyrrolidin-3- | | [M + H]+, ESI pos |
| | codes. | yl)oxy-6-[5-[(6- | | ¹H NMR (300 MHz, |
| | | methylpyridazin-3- | | METHANOL-d4) δ = |
| | | yl)amino]benzimidazol- | | 8.81-8.68 (m, 1H), 8.27- |
| | | 1-yl]-3-pyridyl]ethanol | | 8.00 (m, 3H), 7.73- |
| | | | | 7.60 (m, 1H), 7.56-7.47 |
| | | | | (m, 1H), 7.37 (d, J = 9.1 Hz, |
| | | | | 1H), 7.32-7.25 (m, |
| | | | | 1H), 7.15 (d, J = 9.1 Hz, |
| | | | | 1H), 5.89-5.58 (m, 1H), |
| | | | | 5.27-5.08 (m, 1H), 3.83- |
| | | | | 3.65 (m, 1H), 3.51- |
| | | | | 3.35 (m, 2H), 3.30-3.16 |
| | | | | (m, 2H), 2.55 (s, 3H), |
| | | | | 1.51 (br d, J = 6.4 Hz, |
| | | | | 3H) |

### Examples 291, 292, 295, 296, 310, 312, 318, 327, 328, 330, 334, 336, 338-343, 350-352, 358, 365, 366, 368-370, 380, 387, 392, 395, 397, 398, 401, 404-407

In analogy to Example 64, Examples in the following table were prepared, using the respective building blocks G.X and H.X in step 2.

### Building block G.1: N-(6-methylpyridazin-3-yl)-1H-benzimidazol-5-amine

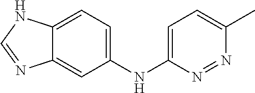 [SMILES: Cc1ccc(Nc2ccc3[nH]cnc3c2)nn1]

Building block G.1 is intermediate 1 in Example 64.

### Building block G.2: N-(6-methyl-4-methylsulfonyl-pyridazin-3-yl)-1˜{H}-benzimidazol-5-amine

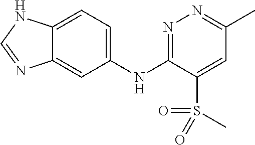 [SMILES: Cc1cc(S(C)(=O)=O)c(Nc2ccc3[nH]cnc3c2)nn1]

### Step 1: 3-chloro-6-methyl-4-methylsulfanyl-pyridazine

A solution of 3,4-dichloro-6-methyl-pyridazine (700 mg, 4.29 mmol, 1.0 eq), sodium thiomethoxide (301 mg, 4.29 mmol, 1.0 eq) in DMF (10 mL) was stirred at 0° C. for 2 hours. The mixture was poured into H₂O (50 mL) and extracted with EtOAc (3×20 ml). The combined organic layers were washed with brine (50 mL), dried over Na₂SO₄, filtered and concentrated. The crude was purified by flash chromatography (SiO₂, 50% EtOAc in PE) to give 3-chloro-6-methyl-4-methylsulfanyl-pyridazine (515 mg, 2.95 mmol, 69% yield) as an off-white solid. LC-MS: m/z=174.7 [M+H]⁺, ESI pos. ¹H NMR (400 MHz, CDCl₃) δ=6.97 (s, 1H), 2.67 (s, 3H), 2.49 (s, 3H).

### Step 2: 3-chloro-6-methyl-4-methylsulfonyl-pyridazine

To a solution of 3-chloro-6-methyl-4-methylsulfanyl-pyridazine (450 mg, 2.58 mmol, 1.0 eq) in DCM (20 mL) was added mCPBA (1.31 g, 6.44 mmol, 2.5 eq) at 0° C. The reaction mixture was stirred at 20° C. for 16 hours. The reaction mixture was poured into saturated aqueous NaHCO₃ (40 mL) under stirring, and was extracted with DCM (3×30 mL). The combined organic layers were dried over Na₂SO₄, filtered and. The residue was purified by preparative TLC (PE/EtOAc 2:1) to give 3-chloro-6-methyl-4-methylsulfonyl-pyridazine (100.0 mg, 0.48 mmol, 18.78% yield) as white solid. LC-MS: m/z=206.9 [M+H]⁺, ESI pos.

### Step 3: N-(6-methyl-4-methylsulfonyl-pyridazin-3-yl)-1H-benzimidazol-5-amine

A mixture of 3-chloro-6-methyl-4-methylsulfonyl-pyridazine (70 mg, 0.34 mmol, 1.0 eq) and 5-aminobenzimidazole (45 mg, 0.34 mmol, 1.0 eq) in iPrOH (3 mL) was stirred at 120° C. for 12 hours. The reaction mixture was concentrated. The residue was purified by preparative TLC (DCM/MeOH 10:1) to give N-(6-methyl-4-methylsulfonyl-pyridazin-3-yl)-1H-benzimidazol-5-amine (30 mg, 0.1 mmol, 29% yield) as a yellow solid. LC-MS: m/z=304.1 [M+H]⁺, ESI pos.

### Building block G.3: 5-(1H-benzimidazol-5-yl)-2-oxa-5-azaspiro[3.4]octane

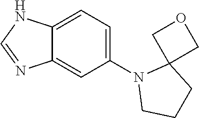 [SMILES: c1nc2cc(N3CCCC34COC4)ccc2[nH]1]

### Step 1: 5-(3,4-dinitrophenyl)-2-oxa-5-azaspiro[3.4]octane

To a solution of 4-fluoro-1,2-dinitro-benzene (100 mg, 0.537 mmol, 1.0 eq) and 2-oxa-5-azaspiro[3.4]octane (61 mg, 0.537 mmol, 1.0 eq) in NMP (2 mL) was added DIEA (141 μL, 0.806 mmol, 1.5 eq). The reaction mixture was stirred for 1 hour at 70° C. The reaction mixture was poured into water (30 mL) and extracted with EtOAc (3×30 mL). The combined organic layers were washed with brine (30 mL), dried over Na₂SO₄, filtered and concentrated. The crude product was purified by flash chromatography (SiO₂, 20-80% EtOAc in heptane) to give 5-(3,4-dinitrophenyl)-2-oxa-5-azaspiro[3.4]octane (124 mg, 0.435 mmol, 81% yield) as a yellow solid. LC-MS: m/z=280.2 [M+H]⁺, ESI pos.

### Step 2: [2-amino-4-(2-oxa-5-azaspiro[3.4]octan-5-yl)phenyl]amine

A solution of 5-(3,4-dinitrophenyl)-2-oxa-5-azaspiro[3.4]octane (124 mg, 0.435 mmol, 1.0 eq) in MeOH (2.24 mL) and THF (2.24 mL) was degassed with argon. Pd/C (22 mg, 0.021 mmol, 0.05 eq) was added. The reaction mixture was degassed with H₂ and stirred with H₂ balloon for 4 h at RT. The reaction mixture was filtered and the filtrate was concentrated to give [2-amino-4-(2-oxa-5-azaspiro[3.4]octan-5-yl)phenyl]amine (80 mg, 0.331 mmol, 76% yield) as a dark green liquid. LC-MS: m/z=220.2 [M+H]⁺, ESI pos.

### Step 3: 5-(1H-benzimidazol-5-yl)-2-oxa-5-azaspiro[3.4]octane

To a solution of [2-amino-4-(2-oxa-5-azaspiro[3.4]octan-5-yl)phenyl]amine (80 mg, 0.329 mmol, 1.0 eq) in EtOH (2.15 mL) was added trimethyl orthoformate (363 uL, 3.29 mmol, 10 eq) and p-toluenesulfonic acid monohydrate (6 mg, 0.033 mmol, 0.10 eq). The reaction mixture was stirred at 80° C. for 1 h. The reaction mixture was concentrated. The residue was suspended in sat. aq. NaHCO₃ (50 mL) and extracted with DCM (3×50 mL). The combined organic layers were washed with brine (50 mL), dried over Na₂SO₄, filtered and concentrated. The crude was purified by flash chromatography (SiO₂, 0-10% MeOH in DCM) to give 5-(1H-benzimidazol-5-yl)-2-oxa-5-azaspiro[3.4]octane (43 mg, 0.188 mmol, 57% yield) as a red solid. LC-MS: m/z=230.2 [M+H]⁺, ESI pos.

### Building block G.4: N-(1H-benzimidazol-5-yl)-4-methyl-2,3-dihydropyridazino[4,5-b][1,4]oxazin-8-amine

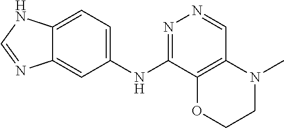 [SMILES: CN1CCOc2c1cnnc2Nc1ccc2[nH]cnc2c1]

### Step 1: 4-methyl-N-[1-(2-trimethylsilylethoxymethyl)benzimidazol-5-yl]-2,3-dihydropyridazino[4,5-b][1,4]oxazin-8-amine

To a solution of 8-chloro-4-methyl-2,3-dihydropyridazino[4,5-b][1,4]oxazine (66643-52-5) (1.0 g, 5.39 mmol, 1.0 eq) in 2-methyl-2-butanol (60.0 mL, 5.39 mmol, 1.0 eq) was added 1-(2-trimethylsilylethoxymethyl)benzimidazol-5-amine (317830-35-6) (1.42 g, 5.39 mmol, 1.0 eq), KOtBu (1.55 g, 16.16 mmol, 3.0 eq) and RuPhos Pd G4 (229.08 mg, 0.27 mmol, 0.05 eq). The reaction mixture was bubbled with N₂ for 10 min and stirred at 100° C. for 16 h. The reaction mixture was cooled to RT, poured into sat. aq. NH₄Cl (100 mL) and extracted with EtOAc (3×50 mL). The combined organic layers were washed with brine (50 mL), dried over Na₂SO₄, filtered and concentrated. The residue was purified by flash chromatography (SiO₂, 0-20% MeOH in DCM) to give 4-methyl-N-[1-(2-trimethylsilylethoxymethyl)benzimidazol-5-yl]-2,3-dihydropyridazino[4,5-b][1,4]oxazin-8-amine (1.2 g, 2.91 mmol, 54% yield) as brown gum. LC-MS: m/z=413.1 [M+H]⁺, ESI pos.

### Step 2: N-(1H-benzimidazol-5-yl)-4-methyl-2,3-dihydropyridazino[4,5-b][1,4]oxazin-8-amine

A solution of 4-methyl-N-[1-(2-trimethylsilylethoxymethyl)benzimidazol-5-yl]-2,3-dihydropyridazino[4,5-b][1,4]oxazin-8-amine (1.2 g, 2.91 mmol, 1.0 eq) in TFA (20 mL) was stirred at 50° C. for 1 h. The reaction mixture was concentrated, poured into sat. aq. NaHCO₃ (50 mL) and extracted with EtOAc (3×50 mL). The combined organic layers were dried over Na₂SO₄, filtered and concentrated. The residue was purified by flash chromatography (SiO₂, 0-20% MeOH in EtOAc) to give N-(1H-benzimidazol-5-yl)-4-methyl-2,3-dihydropyridazino[4,5-b][1,4]oxazin-8-amine (700 mg, 2.48 mmol, 85% yield) as a yellow solid. LC-MS: m/z=283.1 [M+H]⁺, ESI pos. ¹H NMR (400 MHz, METHANOL-d4) δ=8.26 (s, 1H), 8.11 (d, J=1.9 Hz, 1H), 8.07 (s, 1H), 7.52 (d, J=8.8 Hz, 1H), 7.34 (dd, J=1.9, 8.7 Hz, 1H), 4.48-4.43 (m, 2H), 3.48-3.42 (m, 2H), 3.03 (s, 3H).

### Building block G.5: 1-(1H-benzimidazol-5-yl)-N,N-dimethyl-pyrrolidine-2-carboxamide

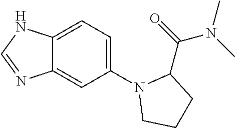 [SMILES: CN(C)C(=O)C1CCCN1c1ccc2[nH]cnc2c1]

Building block G.5 was prepared in analogy to Building block G.3 using N,N-dimethylpyrrolidine-2-carboxamide (433980-61-1) in step 1.

LC-MS: m/z=259.2 [M+H]⁺, ESI pos.

### Building block G.6: (4aS,7aR)-6-(1H-benzimidazol-5-yl)-4-methyl-2,3,4a,5,7,7a-hexahydropyrrolo[3,4-b][1,4]oxazine

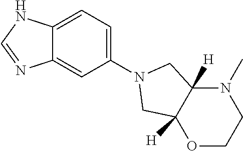 [SMILES: CN1CCO[C@@H]2CN(c3ccc4[nH]cnc4c3)C[C@@H]21]

Building block G.6 was prepared in analogy to Building block G.3 using (4aS,7aR)-4-methyl-3,4a,5,6,7,7a-hexahydro-2H-pyrrolo[3,4-b][1,4]oxazine (1360534-98-O) in step 1.

LC-MS: m/z=259.3 [M+H]⁺, ESI pos.

### Building block G.7: N-(1H-benzimidazol-5-yl)cyclopropanecarboxamide

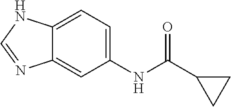 [SMILES: O=C(Nc1ccc2[nH]cnc2c1)C1CC1]

### Step 1: N-[1-(2-trimethylsilylethoxymethyl)benzimidazol-5-yl]cyclopropanecarboxamide

To a solution of cyclopropanecarboxylic acid (196 mg, 2.28 mmol, 1.2 eq) in DMF (5 mL) was added HATU (670 mg, 2.85 mmol, 1.5 eq) and the mixture was stirred at 25° C. for 30 min. DIPEA (1.0 mL, 5.69 mmol, 3.0 eq) was added and the mixture was stirred at 25° C. for 30 min. The reaction mixture was diluted with water (50 mL) and extracted by EtOAc (3×20 mL). The combined organic layers were washed with brine (20 mL), dried over Na₂SO₄, filtered and concentrated. The residue was purified by flash chromatography (SiO₂, 50% EtOAc in PE) to give N-[1-(2-trimethylsilylethoxymethyl)benzimidazol-5-yl]cyclopropanecarboxamide (550 mg, 1.66 mmol, 79% yield) as a brown solid. LC-MS: m/z=332.0 [M+H]⁺, ESI pos.

### Step 2: N-(1H-benzimidazol-5-yl)cyclopropanecarboxamide

To N-[1-(2-trimethylsilylethoxymethyl)benzimidazol-5-yl]cyclopropanecarboxamide (500 mg, 1.51 mmol, 1.0 eq) was added TFA (1.0 mL) and the reaction mixture was stirred at 25° C. for 1 h. The reaction mixture was concentrated to give N-(1H-benzimidazol-5-yl)cyclopropanecarboxamide (268 mg, 1.33 mmol, 88% yield) as brown oil which was used without further purification. LC-MS: m/z=202.1 [M+H]⁺, ESI pos.

### Building block G.8: (3aS,6aS)-1-(1H-benzimidazol-5-yl)-2,3,3a,4,5,6a-hexahydropyrrolo[3,4-b]pyrrol-6-one

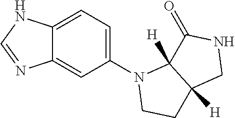 [SMILES: O=C1NC[C@@H]2CCN(c3ccc4[nH]cnc4c3)[C@H]12]

Building block G.8 was prepared in analogy to Building block G.3 using (3aS,6aS)-1-(3,4-diaminophenyl)-2,3,3a,4,5,6a-hexahydropyrrolo[2,3-c]pyrrol-6-one (1523530-51-9) in step 1.

LC-MS: m/z=243.1 [M+H]⁺, ESI pos.

### Building block G.9: 4-(1H-benzimidazol-5-yl)-1-methyl-pyrimidin-2-one

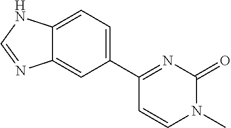 [SMILES: Cn1ccc(-c2ccc3[nH]cnc3c2)nc1=O]

### Step 1:1-methyl-4-[1-(2-trimethylsilylethoxymethyl)benzimidazol-5-yl]pyrimidin-2-one

A mixture of trimethyl-[2-[[5-(4,4,5,5-tetramethyl-1,3,2-dioxaborolan-2-yl)benzimidazol-1-yl]methoxy]ethyl]silane (780 mg, 2.08 mmol, 1.0 eq), 4-chloro-1-methyl-pyrimidin-2-one (361 mg, 2.5 mmol, 1.2 eq), Na₂COₛ(442 mg, 4.17 mmol, 2.0 eq) and Pd(dppf) Cl₂ (152 mg, 0.21 mmol, 0.1 eq) in 1,4-dioxane (2.5 mL) and water (0.5 mL) was stirred at 100° C. for 16 h under N₂. The mixture was concentrated. The residue was purified by flash chromatography (SiO₂, 0-20% MeOH in EtOAc) to give 1-methyl-4-[1-(2-trimethylsilylethoxymethyl)benzimidazol-5-yl]pyrimidin-2-one (407 mg, 1.14 mmol, 49% yield) as a yellow oil. LC-MS: m/z=357.3 [M+H]⁺, ESI pos.

### Step 2: 4-(1H-benzimidazol-5-yl)-1-methyl-pyrimidin-2-one

A mixture of 1-methyl-4-[1-(2-trimethylsilylethoxymethyl)benzimidazol-5-yl]pyrimidin-2-one (450 mg, 1.26 mmol, 1.0 eq) in TFA (5.0 mL) was stirred at 50° C. for 1 h. The mixture was concentrated to give 4-(1H-benzimidazol-5-yl)-1-methyl-pyrimidin-2-one (400 mg, 1.18 mmol, 93% yield) as a yellow solid. LC-MS: m/z=227.2 [M+H]⁺, ESI pos. ¹H NMR (400 MHz, METHANOL-d4) δ=9.44 (s, 1H), 8.64 (s, 1H), 8.36 (dd, J=1.1, 8.8 Hz, 1H), 8.30 (d, J=6.8 Hz, 1H), 7.97 (d, J=8.7 Hz, 1H), 7.23 (d, J=6.8 Hz, 1H), 3.65 (s, 3H).

### Building block G.10: 5-(6-methylpyridazin-3-yl)-1H-benzimidazole

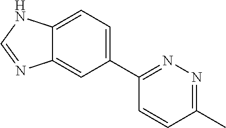 [SMILES: Cc1ccc(-c2ccc3[nH]cnc3c2)nn1]

Building block G.10 was prepared in analogy to Building block G.9 using 4-bromo-3-methoxy-1-methyl-1H-pyrazole (1350323-85-1) in step 1.

LC-MS: m/z=211.1 [M+H]⁺, ESI pos.

### Building block G.11: (2S)-1-(1H-benzimidazol-5-yl)pyrrolidine-2-carbonitrile

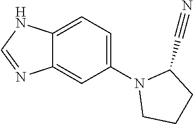 [SMILES: N#C[C@@H]1CCCN1c1ccc2[nH]cnc2c1]

Building block G.11 was prepared in analogy to Building block G.3 using (2S)-2-Pyrrolidinecarbonitrile (CAS: 204387-53-1) in step 1.

LC-MS: m/z=213.4 [M+H]⁺, ESI pos.

### Building block G.12: 4-[[6-(1H-benzimidazol-5-yloxy)pyridazin-3-yl]methyl]morpholine

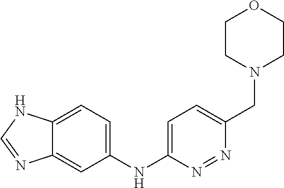 [SMILES: c1nc2cc(Nc3ccc(CN4CCOCC4)nn3)ccc2[nH]1]

### Step 1: 4-[[6-(1-tritylbenzimidazol-5-yl)oxypyridazin-3-yl]methyl]morpholine

A mixture of 1-tritylbenzimidazol-5-ol (100 mg, 0.27 mmol, 1.0 eq), 4-[(6-chloropyridazin-3-yl)methyl]morpholine (57 mg, 0.27 mmol, 1.0 eq), KHCO₃ (53.11 mg, 0.53 mmol, 2.0 eq) and DMF (2 mL) was stirred at 130° C. for 12 hours. The reaction mixture was concentrated and the residue was purified by flash chromatography (SiO₂, 0-20% MeOH in EtOAc) to give 4-[[6-(1-tritylbenzimidazol-5-yl)oxypyridazin-3-yl]methyl]morpholine (70 mg, 0.13 mmol, 48% yield) as yellow solid. LC-MS: m/z=554.1 [M+H]⁺, ESI pos.

Step 2: 4-[[6-(1H-benzimidazol-5-yloxy)pyridazin-3-yl]methyl]morpholine 4-[[6-(1-tritylbenzimidazol-5-yl)oxypyridazin-3-yl]methyl]morpholine (70 mg, 0.13 mmol, 1.0 eq.) was dissolved in TFA (2 mL) and the mixture was stirred at 0° C. for 2 hours. The reaction mixture was concentrated to give 4-[[6-(1H-benzimidazol-5-yloxy)pyridazin-3-yl]methyl]morpholine (0.11 mmol, 85% yield) which was used without further purification. LC-MS: m/z=312.3 [M+H]⁺, ESI pos.

### Building block G.13: 5-(6-methylpyridazin-3-yl)oxy-1H-benzimidazole

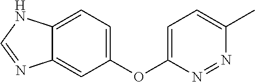 [SMILES: Cc1ccc(Oc2ccc3[nH]cnc3c2)nn1]

Building block G.13 was prepared in analogy to Building block G.12 using 3-chloro-6-methylpyridazine (CAS: 1121-79-5) in step 1.

LC-MS: m/z=227.2 [M+H]⁺, ESI pos. ¹H NMR (400 MHz, DMSO-d6) δ=8.24 (s, 1H), 7.61 (dd, J=4.8, 8.9 Hz, 2H), 7.39 (d, J=2.1 Hz, 1H), 7.30 (d, J=9.0 Hz, 1H), 7.01 (dd, J=2.3, 8.6 Hz, 1H), 3.17 (s, 3H).

### Building block G.14: 5-[6-[(4-methylpiperazin-1-yl)methyl]pyridazin-3-yl]oxy-1H-benzimidazole

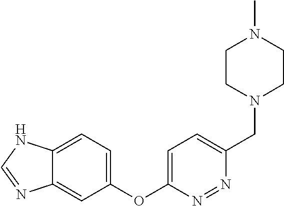 [SMILES: CN1CCN(Cc2ccc(Oc3ccc4[nH]cnc4c3)nn2)CC1]

Building block G.14 was prepared in analogy to Building block G.12 using 3-chloro-6-[(4-methyl-1-piperazinyl)methyl]pyridazine (CAS: 1566269-35-9) in step 1.

LC-MS: m/z=325.2 [M+H]⁺, ESI pos.

### Building block G.15: N-(1H-benzimidazol-5-yl)-2-(1-piperidyl)acetamide

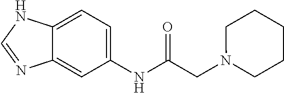 [SMILES: O=C(CN1CCCCC1)Nc1ccc2[nH]cnc2c1]

Building block G.15 was prepared in analogy to Building block G.7 using 1-piperidineacetic acid (CAS: 3235-67-4) in step 1.

LC-MS: m/z=259.1 [M+H]⁺, ESI pos.

### Building block G.16: 2-(1H-benzimidazol-5-yl)-6-methyl-5H-pyrrolo[3,4-b]pyridin-7-one

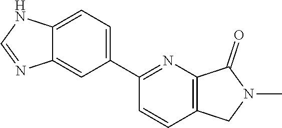 [SMILES: CN1Cc2ccc(-c3ccc4[nH]cnc4c3)nc2C1=O]

Building block G.16 was prepared in analogy to Building block G.9 using 2-chloro-5,6-dihydro-6-methyl-7H-pyrrolo[3,4-b]pyridin-7-one (CAS: 1201924-35-7) in step 1.

LC-MS: m/z=265.2 [M+H]⁺, ESI pos.

### Building block G.17: N-(1H-benzimidazol-5-yl)-1-fluoro-cyclopropanecarboxamide

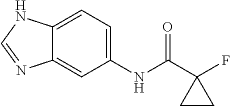 [SMILES: O=C(Nc1ccc2[nH]cnc2c1)C1(F)CC1]

Building block G.17 was prepared in analogy to Building block G.7 using 1-fluorocyclopropanecarboxylic acid (CAS: 137081-41-5) in step 1.

LC-MS: m/z=220.0 [M+H]⁺, ESI pos.

### Building block G.18: 6-fluoro-N-(6-methylpyridazin-3-yl)-1H-benzimidazol-5-amine

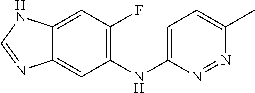 [SMILES: Cc1ccc(Nc2cc3nc[nH]c3cc2F)nn1]

Building block G.18 was prepared in Example 92, step 2.

### Building block G.19: 1-[5-(1H-benzimidazol-5-yl)oxazol-2-yl]pyrrolidin-2-one

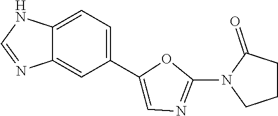 [SMILES: O=C1CCCN1c1ncc(-c2ccc3[nH]cnc3c2)o1]

### Step 1: 4-bromo-N-oxazol-2-yl-butanamide

To a solution of 1,3-oxazol-2-amine (2.00 g, 23.79 mmol, 1.0 eq) and pyridine (5.77 mL, 71.36 mmol, 3.0 eq) in DCM (50 mL) was added 4-bromobutyryl chloride (6.61 g, 35.68 mmol, 1.5 eq) dropwise slowly at 0° C. The reaction mixture was stirred at 0° C. for 1 hour. The reaction mixture was poured into H₂O (200 mL) and extracted with DCM (3×100 mL). The combined organic layers were washed with 0.5N HCl (200 mL), then washed with brine (300 mL), dried over Na₂SO₄, filtered and concentrated to give 4-bromo-N-oxazol-2-yl-butanamide (2.60 g, 11.16 mmol, 47% yield) as yellow oil which was used without further purification. LC-MS: m/z=233.0 [M+H]⁺, ESI pos.

### Step 2: 1-oxazol-2-ylpyrrolidin-2-one

To a solution of 4-bromo-N-oxazol-2-yl-butanamide (2.6 g, 11.16 mmol, 1.0 eq) in DMSO (50 mL) was added NaOAc (2.29 g, 27.89 mmol, 2.5 eq). The reaction mixture was stirred at 100° C. for 30 minutes. The reaction mixture was cooled to RT, concentrated and the residue was purified by flash chromatography (SiO₂, 0-20% MeOH in EtOAc) to give 1-oxazol-2-ylpyrrolidin-2-one (500 mg, 3.29 mmol, 29% yield) as a yellow solid. LC-MS: m/z=153.0 [M+H]⁺, ESI pos. ¹H NMR (400 MHz, METHANOL-d4) δ=7.70 (d, J=0.9 Hz, 1H), 7.08 (d, J=0.8 Hz, 1H), 4.00 (t, J=7.1 Hz, 2H), 2.60 (t, J=8.1 Hz, 2H), 2.21 (quin, J=7.6 Hz, 2H) Step 3: 1-(5-bromooxazol-2-yl)pyrrolidin-2-one

To a solution of 1-oxazol-2-ylpyrrolidin-2-one (500 mg, 3.29 mmol, 1.0 eq) in DMF (17 mL) was added NBS (585 mg, 3.29 mmol, 1.0 eq). The reaction mixture was stirred at 30° C. for 1 hour. The reaction mixture was concentrated and the residue was purified by flash chromatography (SiO₂, 0-20% MeOH in EtOAc) to give 1-(5-bromooxazol-2-yl)pyrrolidin-2-one (400 mg, 1.73 mmol, 53% yield) as a yellow solid. LC-MS: m/z=230.9 [M+H]⁺, ESI pos. ¹H NMR (400 MHz, METHANOL-d4) δ=7.03 (s, 1H), 3.97 (t, J=7.1 Hz, 2H), 2.60 (t, J=8.1 Hz, 2H), 2.26-2.17 (m, 2H).

### Step 4: 1-[5-[1-(2-trimethylsilylethoxymethyl)benzimidazol-5-yl]oxazol-2-yl]pyrrolidin-2-one

To a solution of 1-(5-bromooxazol-2-yl)pyrrolidin-2-one (400 mg, 1.73 mmol, 1.0 eq) and trimethyl-[2-[[5-(4,4,5,5-tetramethyl-1,3,2-dioxaborolan-2-yl)benzimidazol-1-yl]methoxy]ethyl]silane (972 mg, 2.6 mmol, 1.5 eq) in 1,4-dioxane (10 mL) was added Na₂CO₃ (459 mg, 4.33 mmol, 2.5 eq). The mixture was bubbled with N₂ for 10 minutes. Pd(dppf)Cl₂DCM (283 mg, 0.35 mmol, 0.2 eq) and H₂O (2 mL) were added the reaction mixture was stirred at 80° C. for 2 hours under N₂. The reaction mixture was cooled to RT. The reaction mixture was diluted with H₂O (40 mL) and extracted with EtOAc (2×30 mL). The combined organic layers were dried over Na₂SO₄, filtered, and concentrated. The residue was purified by flash chromatography (SiO₂, 0-100% EtOAc) to give 1-[5-[1-(2-trimethylsilylethoxymethyl)benzimidazol-5-yl]oxazol-2-yl]pyrrolidin-2-one (500 mg, 1.25 mmol, 72% yield) as yellow oil. LC-MS: m/z=399.1 [M+H]⁺, ESI pos.

### Step 5: 1-[5-(1H-benzimidazol-5-yl)oxazol-2-yl]pyrrolidin-2-one

1-[5-[1-(2-trimethylsilylethoxymethyl)benzimidazol-5-yl]oxazol-2-yl]pyrrolidin-2-one (500 mg, 1.25 mmol, 1.0 eq) was dissolved with TFA (5.0 mL). The solution was stirred at 20° C. for 1 hour. The reaction mixture was concentrated. The residue was dissolved in ACN (1 mL) and H₂O (10 mL) and the mixture was lyophilized to afford 1-[5-(1H-benzimidazol-5-yl)oxazol-2-yl]pyrrolidin-2-one (400 mg, 1.05 mmol, 83% yield) as yellow solid which was used without further purification. LC-MS: m/z=269.0 [M+H]⁺, ESI pos.

### Building block G.20: N-(6-methylpyridazin-3-yl)-6-(oxetan-3-ylmethyl)-1H-benzimidazol-5-amine

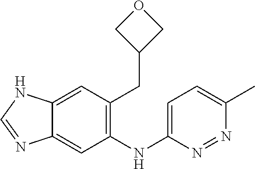 [SMILES: Cc1ccc(Nc2cc3nc[nH]c3cc2CC2COC2)nn1]

### Step 1: tert-butyl N-(5-bromo-4-methyl-2-nitro-phenyl)-N-tert-butoxycarbonyl-carbamate

To a solution of 5-bromo-4-methyl-2-nitro-aniline (25.0 g, 108.2 mmol, 1.0 eq) in DMF (100 mL) was added slowly NaH (5.19 g, 129.84 mmol, 1.2 eq) and the mixture was stirred at 0° C. for 10 min. A solution of di-t-butyldicarbonate (28.34 g, 129.84 mmol, 1.2 eq) in DMF (300 mL) was added dropwise and the mixture was stirred at RT for 14 hours. The reaction mixture was diluted with water (300 mL) and extracted with EtOAc (3×300 mL). The combined organic layers were dried over Na₂SO₄, filtered and concentrated. The residue was purified by flash chromatography (SiO₂, 0-10% EtOAc in PE) and concentrated to give tert-butyl N-(5-bromo-4-methyl-2-nitro-phenyl)-N-tert-butoxycarbonyl-carbamate (26.0 g, 60.29 mmol, 50% yield) as orange solid. ¹H NMR (400 MHz, CDCl₃) δ=7.98 (s, 1H), 7.53 (s, 1H), 2.52 (s, 3H), 1.44 (s, 18H).

### Step 2: tert-butyl N-[5-bromo-4-(bromomethyl)-2-nitro-phenyl]-N-tert-butoxycarbonyl-carbamate

To a solution of tert-butyl N-(5-bromo-4-methyl-2-nitro-phenyl)-N-tert-butoxycarbonyl-carbamate (25.0 g, 57.97 mmol, 1.0 eq) in trifluoromethylbenzene (10 mL) were added N-bromosuccinimide (12.38 g, 69.56 mmol, 1.2 eq) and benzoyl peroxide (1.4 g, 5.8 mmol, 0.1 eq). The reaction mixture was stirred at 80° C. for 16 hours. The reaction mixture was cooled to RT and poured into water (50 mL). The mixture was extracted with DCM (2×100 mL). The combined organic layers were washed with brine (100 mL), dried over Na₂SO₄, filtered, and concentrated. The residue was purified by flash chromatography (SiO₂, 25% EtOAc in PE) to give tert-butyl N-[5-bromo-4-(bromomethyl)-2-nitro-phenyl]-N-tert-butoxycarbonyl-carbamate (23.0 g, 45.08 mmol, 77.77% yield) as a yellow solid. ¹H NMR (400 MHz, CDCl₃) δ=9.74 (br s, 1H), 8.98 (s, 1H), 8.32 (s, 1H), 4.60 (s, 2H), 1.58 (br s, 9H).

### Step 3: tert-butyl N-[5-bromo-2-nitro-4-[(triphenyl-5-phosphanylidene)methyl]phenyl]-N-tert-butoxycarbonyl-carbamate

To a mixture of tert-butyl N-[5-bromo-4-(bromomethyl)-2-nitro-phenyl]-N-tert-butoxycarbonyl-carbamate (7.0 g, 13.72 mmol, 1.0 eq) in toluene (100 mL) was added triphenylphosphine (3.6 g, 13.72 mmol, 1.0 eq). The reaction was stirred at 110° C. for 12 hours. The reaction mixture was cooled to RT and concentrated. The residue was suspended in PE (50 mL), stirred at 30° C. for 1 h and filtered. The filtrate was concentrated to give tert-butyl N-[5-bromo-2-nitro-4-[(triphenyl-,5-phosphanylidene)methyl]phenyl]-N-tert-butoxycarbonyl-carbamate (5.0 g, 7.23 mmol, 53% yield) as a yellow solid. LC-MS: m/z=593.3 [M+H]⁺, ESI pos.

### Step 4: tert-butyl N-[5-bromo-2-nitro-4-(oxetan-3-ylidenemethyl)phenyl]carbamate

To a cooled (0° C.) solution of tert-butyl N-[5-bromo-2-nitro-4-[(triphenyl-,5-phosphanylidene)methyl]phenyl]-N-tert-butoxycarbonyl-carbamate (6.0 g, 8.68 mmol, 1.0 eq) in THF (200 mL) was added KOtBu (1.46 g, 13.01 mmol, 1.5 eq) followed by 3-oxetanone (1.25 g, 17.35 mmol, 2.0 eq) and the reaction was stirred at 0° C. for 2 hours. The reaction mixture was poured into water (50 mL) and extracted with EtOAc (3×50 mL). The combined organic layers were washed with brine (50 mL), dried over Na₂SO₄, filtered and concentrated. The residue was purified by flash chromatography (SiO₂, 25% EtOAc in PE) to afford tert-butyl N-[5-bromo-2-nitro-4-(oxetan-3-ylidenemethyl)phenyl]carbamate (2.0 g, 5.19 mmol, 60% yield) as a yellow solid.

### Step 5: tert-butyl N-[5-[(6-methylpyridazin-3-yl)amino]-2-nitro-4-(oxetan-3-ylidenemethyl)phenyl]carbamate

To a mixture of tert-butyl N-[5-bromo-2-nitro-4-(oxetan-3-ylidenemethyl)phenyl]carbamate (2.00 g, 5.19 mmol, 1.0 eq) and 3-amino-6-methylpyridazine (1.13 g, 10.38 mmol, 2.0 eq) in 1,4-dioxane (50 mL) was added Cs₂CO₃ (5.07 g, 15.58 mmol, 3.0 eq) and [tBuBrettPhos Pd(allyl)]OTf (811 mg, 1.04 mmol, 0.2 eq), and the reaction was stirred at 80° C. for 12 hours under N₂. The reaction mixture was cooled to RT, poured into water (50 mL), and the mixture was extracted with EtOAc (3×50 mL). The combined organic layers were washed with brine (50 mL), dried over Na₂SO₄, filtered and concentrated. The residue was purified by flash chromatography (SiO₂, 0-100% EtOAc in PE) to give tert-butyl N-[5-[(6-methylpyridazin-3-yl)amino]-2-nitro-4-(oxetan-3-ylidenemethyl)phenyl]carbamate (2.10 g, 5.08 mmol, 97% yield) as a brown solid. LC-MS: m/z=414.2 [M+H]⁺, ESI pos.

### Step 6: tert-butyl N-[2-amino-5-[(6-methylpyridazin-3-yl)amino]-4-(oxetan-3-ylmethyl)phenyl]carbamate

To a mixture of tert-butyl N-[5-[(6-methylpyridazin-3-yl)amino]-2-nitro-4-(oxetan-3-ylidenemethyl)phenyl]carbamate (200 mg, 0.48 mmol, 1.0 eq) in MeOH (10 mL) was added Pd/C (51 mg, 0.05 mmol, 0.1 eq) and the reaction was stirred at 30° C. for 3 hours under H₂ balloon. The reaction mixture was filtrated and the filtrate was concentrated to give tert-butyl N-[2-amino-5-[(6-methylpyridazin-3-yl)amino]-4-(oxetan-3-ylmethyl)phenyl]carbamate (150 mg, 0.39 mmol, 80% yield) as an off white oil which was used without further purification. LC-MS: m/z=386.2 [M+H]⁺, ESI pos.

### Step 7: N4-(6-methylpyridazin-3-yl)-5-(oxetan-3-ylmethyl)benzene-1,2,4-triamine

To a solution of tert-butyl N-[2-amino-5-[(6-methylpyridazin-3-yl)amino]-4-(oxetan-3-ylmethyl)phenyl]carbamate (150 mg, 0.39 mmol, 1.0 eq) in DCM (2 mL) was added TFA (1.0 mL) and the reaction was stirred at 30° C. for 2 hours. The reaction mixture was concentrated to give N4-(6-methylpyridazin-3-yl)-5-(oxetan-3-ylmethyl)benzene-1,2,4-triamine (110 mg, 0.39 mmol, 99% yield) as a yellow oil which was used without further purification. LC-MS: m/z=286.1 [M+H]⁺, ESI pos.

### Step 8: N-(6-methylpyridazin-3-yl)-6-(oxetan-3-ylmethyl)-1H-benzimidazol-5-amine

To a solution of N4-(6-methylpyridazin-3-yl)-5-(oxetan-3-ylmethyl)benzene-1,2,4-triamine (150 mg, 0.53 mmol, 1.0 eq) in MeOH (2 mL) were added trimethyl orthoformate (574 μL, 5.26 mmol, 10 eq) and TsOH (9 mg, 0.05 mmol, 0.1 eq). The solution was stirred at 80° C. for 2 hours. The reaction mixture was poured into water (50 mL) and extracted with EtOAc (3×50 mL). The combined organic layers were washed with brine (50 mL), dried over Na₂SO₄, filtered and concentrated. The residue was purified by preparative TLC (DCM:MeOH 10:1) to afford N-(6-methylpyridazin-3-yl)-6-(oxetan-3-ylmethyl)-1H-benzimidazol-5-amine (100 mg, 0.34 mmol, 64% yield) as a yellow oil. LC-MS: m/z=296.2 [M+H]⁺, ESI pos.

### Building block G.21: 5-(3-methoxy-1-methyl-pyrazol-4-yl)-1˜{H}-benzimidazole

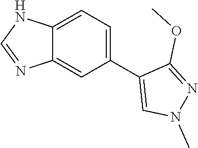 [SMILES: COc1nn(C)cc1-c1ccc2[nH]cnc2c1]

Building block G.21 was prepared in analogy to Building block G.9 using 2-chloro-5,6-dihydro-6-methyl-7H-pyrrolo[3,4-b]pyridin-7-one (CAS: 1201924-35-7) in step 1.

LC-MS: m/z=265.2 [M+H]⁺, ESI pos.

### Building block H.1: 1-(3-acetyl-6-chloro-2-pyridyl)-5-methyl-6,7-dihydro-4˜{H}-pyrazolo[4,3-c]pyridine-3-carbonitrile

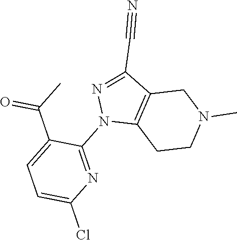 [SMILES: CC(=O)c1ccc(Cl)nc1-n1nc(C#N)c2c1CCN(C)C2]

To a stirred solution of 4,5,6,7-Tetrahydro-5-methyl-1H-pyrazolo[4,3-c]pyridine-3-carbonitrile (1522689-76-4) (89 mg, 0.544 mmol, 1.0 eq) and 1-(6-chloro-2-fluoro-3-pyridyl)ethanone (94 mg, 0.544 mmol, 1.0 eq) at RT in DMSO (3 mL) under an argon atmosphere was added DIPEA (190 uL, 1.09 mmol, 2.0 eq). The reaction mixture was stirred at RT for 5 hours. The reaction mixture was poured into H2O (50 mL) and extracted with EtOAc (3×40 ml). The combined organic layers were washed with brine (30 ml), dried over Na₂SO₄, filtered and concentrated. The residue was purified by flash chromatography (SiO₂, 0-50% EtOAc in heptane) to give 1-(3-acetyl-6-chloro-2-pyridyl)-5-methyl-6,7-dihydro-4˜{H}-pyrazolo[4,3-c]pyridine-3-carbonitrile (63 mg, 0.386 mmol, 71% yield) as a white foam. LC-MS: m/z=163.2 [M+H]⁺, ESI pos.

### Building block H.2: 1-(3-acetyl-6-chloro-2-pyridyl)-5-methyl-pyrazole-3-carbonitrile

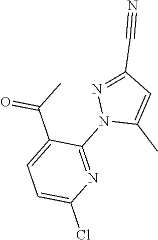 [SMILES: CC(=O)c1ccc(Cl)nc1-n1nc(C#N)cc1C]

Building block H.2 is prepared in Example 64, step 1.

### Building block H.3: methyl 6-chloro-2-[3-(difluoromethyl)-5-methyl-pyrazol-1-yl]pyridine-3-carboxylate

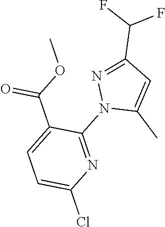 [SMILES: COC(=O)c1ccc(Cl)nc1-n1nc(C(F)F)cc1C]

To a solution of 6-chloro-2-fluoro-nicotinic acid methyl ester (500 mg, 2.64 mmol, 1.0 eq) in DMF (4.21 mL) was added 3-(difluoromethyl)-5-methyl-1H-pyrazole (418 mg, 3.17 mmol, 1.2 eq) and K₂CO₃ (547 mg, 3.96 mmol, 1.5 eq). The reaction mixture was stirred at RT for 5 hours. The reaction was diluted with H₂O (50 mL) and extracted with EtOAc (3×30 mL). The combined organic layers were washed with brine (30 mL), dried over Na₂SO₄, filtered and concentrated. The residue was purified by flash chromatography (SiO₂, 0-50% EtOAc in heptane) to give methyl 6-chloro-2-[3-(difluoromethyl)-5-methyl-pyrazol-1-yl]pyridine-3-carboxylate (315 mg, 1.04 mmol, 40% yield) as a colorless oil. LC-MS: m/z=302.1 [M+H]⁺, ESI pos. ¹H NMR (600 MHz, CDCl₃) δ ppm 8.12 (d, J=8.1 Hz, 1H), 7.45 (d, J=8.1 Hz, 1H), 6.53-6.72 (m, 1H), 6.42-6.45 (m, 1H), 3.73 (s, 3H), 2.55 (d, J=0.8 Hz, 3H).

### Building block H.4: 1-[6-chloro-2-[3-methyl-1-(2,2,2-trifluoroethyl)pyrazol-4-yl]-3-pyridyl]ethanone

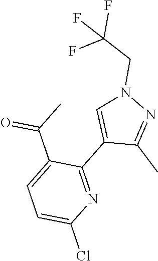 [SMILES: CC(=O)c1ccc(Cl)nc1-c1cn(CC(F)(F)F)nc1C]

A mixture of 3-methyl-4-(4,4,5,5-tetramethyl-1,3,2-dioxaborolan-2-yl)-1-(2,2,2-trifluoroethyl)pyrazole (Building block A.10) (3.71 g, 12.79 mmol, 1.0 eq), 1-(2-bromo-6-chloro-3-pyridyl)ethanone (3.0 g, 12.79 mmol, 1.0 eq), K₂CO₃ (3.54 g, 25.59 mmol, 2.0 eq) and Pd(dppf)Cl₂CH₂Cl₂ (1.04 g, 1.28 mmol, 0.1 eq) in 1,4-dioxane (60 mL) and water (6 mL) was stirred at 80° C. under N₂ atmosphere for 12 h. The mixture was cooled to RT and concentrated. The residue was purified by flash chromatography (SiO₂, 0-25% EtOAc in heptane) to give 1-[6-chloro-2-[3-methyl-1-(2,2,2-trifluoroethyl)pyrazol-4-yl]-3-pyridyl]ethanone (3.2 g, 10.07 mmol, 79% yield) as a yellow oil. LC-MS: m/z=318.1 [M+H]⁺, ESI pos.

### Building block H.5:1-[6-chloro-2-[3-(difluoromethyl)-5-ethyl-pyrazol-1-yl]-3-pyridyl]ethanone

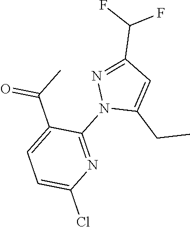 [SMILES: CCc1cc(C(F)F)nn1-c1nc(Cl)ccc1C(C)=O]

To a solution of 3-(difluoromethyl)-5-ethyl-1H-pyrazole (1015779-27-7) (192 mg, 1.31 mmol, 1.2 eq) in DMF (3 mL) was added 1-(6-chloro-2-fluoro-3-pyridyl)ethanone (150 mg, 0.864 mmol, 1.0 eq) and Cs₂CO₃ (563 mg, 1.73 mmol, 2.0 eq). The reaction mixture was stirred at RT for 16 hours. The reaction mixture was diluted with H₂O (30 mL) and extracted with DCM (3×30 mL). The combined organic layers were washed with brine (30 mL), dried over Na₂SO₄, filtered and concentrated. The residue was purified by flash chromatography (SiO₂, 0-100% EtOAc in heptane) to give 1-[6-chloro-2-[3-(difluoromethyl)-5-ethyl-pyrazol-1-yl]-3-pyridyl]ethanone (118 mg, 0.34 mmol, 25.54%) as light brown solid. LC-MS: m/z=300.0 [M+H]⁺, ESI pos. ¹H NMR (600 MHz, DMSO-d6) δ ppm 8.21 (d, J=8.1 Hz, 1H), 7.81 (d, J=8.1 Hz, 1H), 7.01 (t, J=54.3 Hz, 1H), 6.71 (s, 1H), 2.92 (dd, J=7.6, 0.7 Hz, 2H), 2.01 (s, 3H), 1.19-1.25 (m, 4H).

### Building block H.6:1-[2-[3,5-bis(difluoromethyl)pyrazol-1-yl]-6-chloro-3-pyridyl]ethanone

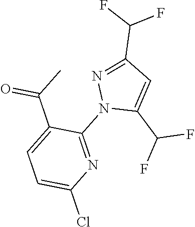 [SMILES: CC(=O)c1ccc(Cl)nc1-n1nc(C(F)F)cc1C(F)F]

1-(6-chloro-2-fluoro-3-pyridyl)ethanone (10 g, 57.61 mmol, 1.0 eq) was dissolved in DMF (200 mL) under Ar. Cs₂CO₃ (37.54 g, 115.23 mmol, 2.000 eq) was added and the mixture was cooled to −60° C. A solution of 3,5-bis(difluoromethyl)-1H-pyrazole (10.17 g, 60.49 mmol, 1.05 eq) in DMF (50 mL) was added dropwise. The reaction mixture was slowly warmed to 0° C. while stirring over 4 hours. The reaction mixture was poured into H₂O (1000 mL) water and extracted with EtOAc (3×250 mL). The combined organic layers were washed with water (200 mL), brine (200 mL), dried over Na₂SO₄, filtered and concentrated. The residue was purified by flash chromatography (SiO₂, 0-60% EtOAc in heptane) to give 1-[2-[3,5-bis(difluoromethyl)pyrazol-1-yl]-6-chloro-3-pyridyl]ethanone (14.7 g, 45.51 mmol, 79% yield) as a light yellow oil. LC-MS: m/z=322.0 [M+H]⁺, ESI pos. ¹H NMR (600 MHz, CDCl₃) δ ppm 7.90 (d, J=8.1 Hz, 1H), 7.54 (br t, J=55.0 Hz, 1H), 7.45 (d, J=8.1 Hz, 1H), 7.04 (s, 1H), 6.68 (t, J=54.4 Hz, 1H), 2.77-3.34 (m, 1H), 2.17 (s, 3H).

### Building block H. 7: 1-[6-chloro-2-[3-(difluoromethyl)-5-methoxy-pyrazol-1-yl]-3-pyridyl]ethanone

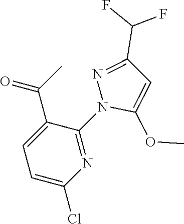 [SMILES: COc1cc(C(F)F)nn1-c1nc(Cl)ccc1C(C)=O]

### Step 1: 6-chloro-2-fluoro-3-(2-methyl-1,3-dioxolan-2-yl)pyridine

To a solution of 1-(6-chloro-2-fluoro-3-pyridyl)ethanone (5.0 g, 28.81 mmol, 1.0 eq) in toluene (100 mL) was added ethylene glycol (4.82 mL, 86.42 mmol, 3.0 eq) and TosOH (496 mg, 2.88 mmol, 0.1 eq). The reaction was stirred at reflux overnight with azeotropic removal of H₂O via Dean Stark trap. The reaction mixture was cooled to RT and concentrated to dryness. The residue was taken up in EtOAc (100 ml) and the solution was washed with H2O (2×50 ml) and brine (50 ml). The organic layer was dried over Na2SO4, filtered and concentrated. The residue was purified by flash chromatography (SiO₂, 10% EtOAc in PE) to give 6-chloro-2-fluoro-3-(2-methyl-1,3-dioxolan-2-yl)pyridine (5.5 g, 25.27 mmol, 88% yield) as a white solid. LC-MS: m/z=218.0 [M+H]⁺, ESI pos. ¹H NMR (400 MHz, METHANOL-d4) δ=8.02 (dd, J=7.9, 9.6 Hz, 1H), 7.46-7.25 (m, 1H), 4.12-4.06 (m, 2H), 3.87-3.78 (m, 2H), 1.69 (d, J=0.7 Hz, 3H).

### Step 2: [6-chloro-3-(2-methyl-1,3-dioxolan-2-yl)-2-pyridyl]hydrazine

A mixture of 6-chloro-2-fluoro-3-(2-methyl-1,3-dioxolan-2-yl)pyridine (5.0 g, 22.98 mmol, 1.0 eq) and hydrazine hydrate (50.0 mL) was stirred at 50° C. for 2 h The reaction mixture cooled to RT, poured into water (50 mL) and extracted with EtOAc (3×50 mL). The combined organic layers were washed with brine (50 mL), dried over anhydrous Na₂SO₄, filtered and concentrated. The residue was purified by preparative TLC (PE:EtOAc 1:1) to give [6-chloro-3-(2-methyl-1,3-dioxolan-2-yl)-2-pyridyl]hydrazine (5.0 g, 21.77 mmol, 95% yield) as a white solid. LC-MS: m/z=230.0 [M+H]⁺, ESI pos.

### Step 3: 2-[6-chloro-3-(2-methyl-1,3-dioxolan-2-yl)-2-pyridyl]-3-(difluoromethyl)-1H-pyrazol-5-one

A mixture of [6-chloro-3-(2-methyl-1,3-dioxolan-2-yl)-2-pyridyl]hydrazine (500 mg, 2.18 mmol, 1.0 eq) and ethyl 4,4-difluoro-3-oxobutanoate (362 mg, 2.18 mmol, 1.0 eq) in toluene (10 mL) was stirred at 130° C. for 12 hours. The reaction mixture cooled to RT, poured into H₂O (100 mL) and extracted with EtOAc (3×100 mL). The combined organic layers were washed with brine (50 mL), dried over Na₂SO₄, filtered and concentrated. The residue was purified by preparative HPLC (Phenomenex luna C18 150×40 mm×15 μm, water with FA-ACN) to give 2-[6-chloro-3-(2-methyl-1,3-dioxolan-2-yl)-2-pyridyl]-3-(difluoromethyl)-1H-pyrazol-5-one (90 mg, 0.27 mmol, 12% yield) as a white solid. LC-MS: m/z=331.8 [M+H]⁺, ESI pos. ¹H NMR (400 MHz, DMSO-d6) δ=12.15-11.52 (m, 1H), 8.24-8.11 (d, 1H), 7.85-7.69 (d, 1H), 7.02-6.60 (t, 1H), 5.35 (s, 1H), 3.93-3.80 (m, 2H), 3.54 (br s, 2H), 1.70-1.53 (s, 3H).

### Step 4: 6-chloro-2-[3-(difluoromethyl)-5-methoxy-pyrazol-1-yl]-3-(2-methyl-1,3-dioxolan-2-yl)pyridine

To a mixture of 2-[6-chloro-3-(2-methyl-1,3-dioxolan-2-yl)-2-pyridyl]-5-(difluoromethyl)-1H-pyrazol-3-one (70 mg, 0.21 mmol, 1.0 eq) in DMF (1 mL) was added iodomethane (30 mg, 0.21 mmol, 1.0 eq). The reaction mixture was stirred at 30° C. for 1 hour. The reaction mixture was cooled to RT, poured into water (50 mL) and extracted with EtOAc (3×50 mL). The combined organic layers were washed with brine (50 mL3), dried over Na₂SO₄, filtered and concentrated. The residue was purified by preparative TLC (PE:EtOAc 2:1) to give 6-chloro-2-[3-(difluoromethyl)-5-methoxy-pyrazol-1-yl]-3-(2-methyl-1,3-dioxolan-2-yl)pyridine (20 mg, 0.06 mmol, 27% yield) as a white solid. LC-MS: m/z=346.6 [M+H]⁺, ESI pos. ¹H NMR (400 MHz, METHANOL-d4) δ=8.13-8.02 (m, 1H), 7.62-7.50 (m, 1H), 6.72-6.34 (m, 1H), 5.97-5.86 (m, 1H), 3.86-3.83 (m, 3H), 3.82-3.75 (m, 2H), 3.59-3.51 (m, 2H), 1.56-1.46 (m, 3H).

### Step 5: 1-[6-chloro-2-[3-(difluoromethyl)-5-methoxy-pyrazol-1-yl]-3-pyridyl]ethanone

To a solution of 6-chloro-2-[3-(difluoromethyl)-5-methoxy-pyrazol-1-yl]-3-(2-methyl-1,3-dioxolan-2-yl)pyridine (80 mg, 0.23 mmol, 1.0 eq) in THF (1 mL) was added 1 N HCl (1.0 mL) and the reaction was stirred at 70° C. for 12 hours. The reaction mixture was cooled to RT, poured into water (50 mL) and extracted with EtOAc (3×50 mL). The combined organic layers were washed with (50 mL), dried over Na₂SO₄, filtered and concentrated to give 1-[6-chloro-2-[3-(difluoromethyl)-5-methoxy-pyrazol-1-yl]-3-pyridyl]ethanone (60 mg, 0.2 mmol, 86% yield) as a yellow oil which was used without further purification. LC-MS: m/z=301.9 [M+H]⁺, ESI pos.

### Building block H.8: 1-[6-chloro-2-[2-methyl-5-(2,2,2-trifluoroethyl)-1,2,4-triazol-3-yl]-3-pyridyl]ethanone

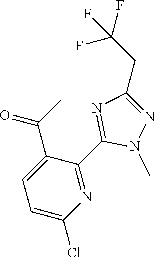 [SMILES: CC(=O)c1ccc(Cl)nc1-c1nc(CC(F)(F)F)nn1C]

### Step 1: N-[(Z)-[amino-(3-bromo-2-pyridyl)methylene]amino]-3,3,3-trifluoro-propanamide

To a solution of 3,3,3-trifluoropropionic acid (4.5 g, 35.15 mmol, 1.2 eq) in DMF (20 mL) were added N′-amino-3-bromo-pyridine-2-carboxamidine (814263-27-9) (6.3 g, 29.3 mmol, 1.0 eq) followed by HATU (10.34 g, 43.94 mmol, 1.5 eq) and DIPEA (10.21 mL, 58.59 mmol, 2.0 eq) and the reaction mixture was stirred at 30° C. for 1 hour. The reaction mixture was poured into water (100 mL) and extracted with EtOAc (3×80 mL). The combined organic layers were washed with brine (60 mL), dried over Na₂SO₄, filtered and concentrated. The residue was purified by flash chromatography (SiO₂, 100% EtOAc) to give N-[(Z)-[amino-(3-bromo-2-pyridyl)methylene]amino]-3,3,3-trifluoro-propanamide (4.0 g, 12.3 mmol, 42% yield) a light yellow solid. LC-MS: m/z=324.9 [M+H]⁺, ESI pos.

### Step 2: 3-bromo-2-[3-(2,2,2-trifluoroethyl)-1H-1,2,4-triazol-5-yl]pyridine

A solution of N-[(Z)-[amino-(3-bromo-2-pyridyl)methylene]amino]-3,3,3-trifluoro-propanamide (3.9 g, 12.0 mmol, 1.0 eq) in ethylene glycol (7.45 g, 119.97 mmol, 10.0 eq) was stirred at 180° C. for 1 hour. The reaction mixture was cooled to RT and poured into water (50 mL). The mixture was extracted with EtOAc (3×50 mL). The combined organic layers were washed with brine (50 mL), dried over Na₂SO₄, filtered and concentrated. The residue was purified by flash chromatography (SiO₂, 0-25% EtOAc in PE) to give 3-bromo-2-[3-(2,2,2-trifluoroethyl)-1H-1,2,4-triazol-5-yl]pyridine (2.0 g, 6.51 mmol, 54% yield) as a light brown solid. LC-MS: m/z=306.9 [M+H]⁺, ESI pos. ¹H NMR (400 MHz, METHANOL-d4) δ=8.69 (dd, J=1.4, 4.6 Hz, 1H), 8.25 (dd, J=1.4, 8.2 Hz, 1H), 7.46 (dd, J=4.6, 8.1 Hz, 1H), 3.83 (q, J=10.4 Hz, 2H).

### Step 3: 3-bromo-2-[2-methyl-5-(2,2,2-trifluoroethyl)-1,2,4-triazol-3-yl]pyridine

To a solution of 3-bromo-2-[3-(2,2,2-trifluoroethyl)-1H-1,2,4-triazol-5-yl]pyridine (2.0 g, 6.51 mmol, 1.0 eq) in DMF (20 mL) were added K₂CO₃ (1.36 g, 9.77 mmol, 1.5 eq) followed by iodomethane (1.39 g, 9.77 mmol, 1.5 eq). The reaction mixture was at 30° C. for 2 hours. The reaction mixture was poured into water (40 mL) and extracted with EtOAc (2×50 mL). The combined organic layers were washed with brine (30 mL), dried over Na₂SO₄, filtered and concentrated. The residue was purified by flash chromatography (SiO₂, 0-25% EtOAc in PE) to give 3-bromo-2-[2-methyl-5-(2,2,2-trifluoroethyl)-1,2,4-triazol-3-yl]pyridine (500 mg, 1.56 mmol, 60% yield) as a light brown. LC-MS: m/z=320.9 [M+H]⁺, ESI pos.

### Step 4: 3-(1-ethoxyvinyl)-2-[2-methyl-5-(2,2,2-trifuoroethyl)-1,2,4-triazol-3-yl]pyridine

To a solution of 3-bromo-2-[2-methyl-5-(2,2,2-trifluoroethyl)-1,2,4-triazol-3-yl]pyridine (500 mg, 1.56 mmol, 1.0 eq) in toluene (20 mL) were added tributyl(1-ethoxyvinyl)tin (844 mg, 2.34 mmol, 1.5 eq) and bis(triphenylphosphine)palladium(II) chloride (82 mg, 0.12 mmol, 0.08 eq). The reaction mixture was bubbled with N₂ for 10 minutes and stirred at 110° C. for 16 hours under N₂. The mixture reaction was cooled to RT and quenched with saturated aqueous KF (50 ml) and stirred at room temperature for 16 hours. The mixture was extracted with EtOAc (3×50 mL). The combined organic layers were washed with brine (30 mL), dried over Na₂SO₄, filtered and concentrated to give 3-(1-ethoxyvinyl)-2-[2-methyl-5-(2,2,2-trifluoroethyl)-1,2,4-triazol-3-yl]pyridine (450 mg, 1.44 mmol, 93% yield) as light brown oil which was used without further purification. LC-MS: m/z=313.0 [M+H]⁺, ESI pos.

### Step 5: 1-[2-[2-methyl-5-(2,2,2-trifluoroethyl)-1,2,4-triazol-3-yl]-3-pyridyl]ethanone

To a solution of 3-(1-ethoxyvinyl)-2-[2-methyl-5-(2,2,2-trifluoroethyl)-1,2,4-triazol-3-yl]pyridine (450 mg, 1.44 mmol, 1.0 eq) in THF (5 mL) was added aq. 1 N HCl (5 mL) and the mixture was stirred at RT for 2 hours. The reaction mixture was poured into saturated aqueous NaHCO₃ (10 mL). The mixture was extracted with EtOAc (2×20 mL). The combined organic layers were washed with brine (10 mL), dried over Na₂SO₄, filtered and concentrated. The residue was purified by flash chromatography (SiO₂, 0-25% EtOAc in PE) to give 1-[2-[2-methyl-5-(2,2,2-trifluoroethyl)-1,2,4-triazol-3-yl]-3-pyridyl]ethanone (400 mg, 1.41 mmol, 98% yield) as light brown solid. LC-MS: m/z=284.9 [M+H]⁺, ESI pos. ¹H NMR (400 MHz, CDCl₃) δ=8.75 (dd, J=1.6, 4.8 Hz, 1H), 7.85 (dd, J=1.7, 7.9 Hz, 1H), 7.46 (dd, J=4.9, 7.8 Hz, 1H), 4.21 (s, 3H), 3.57 (q, J=10.3 Hz, 2H), 2.45 (s, 3H).

### Step 6: 1-[2-[2-methyl-5-(2,2,2-trifluoroethyl)-1,2,4-triazol-3-yl]-1-oxido-pyridin-1-ium-3-yl]ethanone

To a solution of 1-[2-[2-methyl-5-(2,2,2-trifluoroethyl)-1,2,4-triazol-3-yl]-3-pyridyl]ethanone (450 mg, 1.58 mmol, 1.0 eq) in aq. H₂O₂(10.0 mL) was added acetic acid (5 mL). The reaction mixture was stirred at 85° C. for 4 hours. The reaction mixture was cooled to RT, diluted with saturated Na₂SO₃ (30 mL) and stirred for 1 hour. The resulting mixture was extracted with DCM (3×50 mL) and the combined organic layers were dried over Na₂SO₄, filtered and concentrated. The residue was purified by flash chromatography (SiO₂, 0-10% MeOH in DCM) to give 1-[2-[2-methyl-5-(2,2,2-trifluoroethyl)-1,2,4-triazol-3-yl]-1-oxido-pyridin-1-ium-3-yl]ethanone (260 mg, 0.87 mmol, 55% yield) as an off white solid. LC-MS: m/z=301.0 [M+H]⁺, ESI pos.

### Step 7: 1-[6-chloro-2-[2-methyl-5-(2,2,2-trifluoroethyl)-1,2,4-triazol-3-yl]-3-pyridyl]ethanone

A solution of 1-[2-[2-methyl-5-(2,2,2-trifluoroethyl)-1,2,4-triazol-3-yl]-1-oxido-pyridin-1-ium-3-yl]ethanone (260 mg, 0.87 mmol, 1.0 eq) in POCl₃ (6.0 mL) was stirred at 80° C. for 2 hours. The reaction mixture was cooled to RT and concentrated. The concentrated residue was slowly quenched by dropwise addition to sat. aq. NaHCO₃ (30 mL) at RT with vigorous stirring. The mixture was extracted with EtOAc (3×50 mL) and the combined organic layers were dried over Na₂SO₄, filtered and concentracted. The residue was purified by flash chromatography (SiO₂, 0-40% EtOAc in PE) to give 1-[6-chloro-2-[2-methyl-5-(2,2,2-trifluoroethyl)-1,2,4-triazol-3-yl]-3-pyridyl]ethanone (150 mg, 0.47 mmol, 54% yield) as an off white solid. LC-MS: m/z=318.9 [M+H]⁺, ESI pos.

### Building block H.9: 1-[6-chloro-2-[3-(difluoromethoxy)-5-(difluoromethyl)pyrazol-1-yl]-3-pyridyl]ethanone

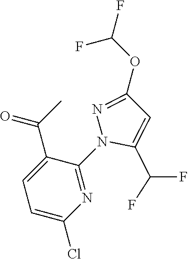 [SMILES: CC(=O)c1ccc(Cl)nc1-n1nc(OC(F)F)cc1C(F)F]

### Step 1: 3-(difluoromethyl)-5-hydroxy-pyrazole-1-carboxylic acid tert-butyl ester

Di-tert-butyl dicarbonate (1.22 g, 1.3 mL, 5.58 mmol, 1.2 eq) was added to a suspension of 3-(difluoromethyl)-1H-pyrazol-5-ol (2416700-52-O) (800 mg, 5.97 mmol, 1.0 eq) in THF (15 mL). A solution of NaHCO₃ (1.17 g, 13.95 mmol, 3.0 eq) in H₂O (15 mL) and 4-dimethylaminopyridine (9.11 mg, 74.58 μmol, 0.10 eq) were added and the reaction mixture stirred at RT for 16 hours. The reaction was poured into sat. aq. NH₄Cl and the mixture was extracted with EtOAc/DCM 1:1 (3×50 mL). The combined organic layers were washed with brine (50 mL), dried over Na₂SO₄, filtered and concentrated to give 3-(difluoromethyl)-5-hydroxy-pyrazole-1-carboxylic acid tert-butyl ester (1.4 g, 4.48 mmol, 75% yield) which was used without further purification. LC-MS: m/z=135.0 [M+H-Boc]⁺, ESI pos.

### Step 2: 5-(difluoromethoxy)-3-(difluoromethyl)pyrazole-1-carboxylic acid tert-butyl ester

To a mixture of 3-(difluoromethyl)-5-hydroxy-pyrazole-1-carboxylic acid tert-butyl ester (283 mg, 1.21 mmol, 1.0 eq) in DMF (12 mL) were added Cs₂CO₃ (1.18 g, 3.63 mmol, 3.0 eq) and sodium chlorodifluoroacetate (276 mg, 1.81 mmol, 1.5 eq). The mixture was stirred at 80° C. for 2 hours. The reaction mixture was cooled to RT, poured into H2O (50 mL) and extracted with EtOAc (3×50 mL). The combined organic layers was washed with H2O (2×20 mL), brine (50 mL), dried over Na₂SO₄, filtered and concentrated to give 5-(difluoromethoxy)-3-(difluoromethyl)pyrazole-1-carboxylic acid tert-butyl ester (1.7 g, quant. yield) as a light brown solid which was used without further purification.

### Step 3: 5-(difluoromethoxy)-3-(difluoromethyl)-1H-pyrazole

5-(difluoromethoxy)-3-(difluoromethyl)pyrazole-1-carboxylic acid tert-butyl ester (1.7 g, 5.98 mmol, 1.0 eq) was dissolved in DCM (15 mL) and TFA (2.3 mL, 29.91 mmol, 5.0 eq) was added. The reaction mixture was stirred at RT for 16 hours. The solvent reaction mixture was concentrated to give 5-(difluoromethoxy)-3-(difluoromethyl)-1H-pyrazole (1.1 g, quant. yield) which was used without further purification.

### Step 4: 1-[6-chloro-2-[3-(difluoromethoxy)-5-(difluoromethyl)pyrazol-1-yl]-3-pyridyl]ethanone

5-(difluoromethoxy)-3-(difluoromethyl)-1H-pyrazole (1.1 g, 5.96 mmol, 1.15 eq) was dissolved in DMF (17.28 mL) under argon. 1-(6-chloro-2-fluoro-3-pyridyl)ethanone (0.90 g, 5.19 mmol, 1.0 eq) and Cs₂CO₃ (8.45 g, 25.93 mmol, 5.0 eq) were added and the reaction mixture was stirred at RT for 1 hour. The reaction mixture was poured into H2O (100 mL) and the mixture was extracted with EtOAc (3×50 mL). The combined organic layers were washed with water (2×30 mL), brine (100 mL), dried over Na₂SO₄, filtered and concentrated. The residue was purified by flash chromatography (SiO₂, 10-100% EtOAc in heptane) to give 1-[6-chloro-2-[3-(difluoromethoxy)-5-(difluoromethyl)pyrazol-1-yl]-3-pyridyl]ethanone (220 mg, 0.60 mmol, 10% yield) as a white solid. LC-MS: m/z=338.1 [M+H]⁺, ESI pos.

### Building block H.10: 1-[6-chloro-2-[3-(difluoromethoxy)-5-methyl-pyrazol-1-yl]-3-pyridyl]ethanone

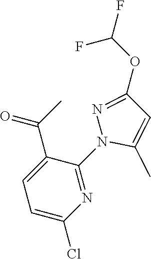 [SMILES: CC(=O)c1ccc(Cl)nc1-n1nc(OC(F)F)cc1C]

Building block H.10 was prepared as building block D.4.

### Building block H.11: 1-[6-chloro-2-[3-(difluoromethyl)-5-methyl-pyrazol-1-yl]-3-pyridyl]ethanone

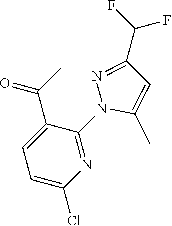 [SMILES: CC(=O)c1ccc(Cl)nc1-n1nc(C(F)F)cc1C]

Building block H.11 was prepared as building block D.3.

### Building block H.12: 1-[6-chloro-2-[3-(difluoromethyl)-4,5-dimethyl-pyrazol-1-yl]-3-pyridyl]ethanone

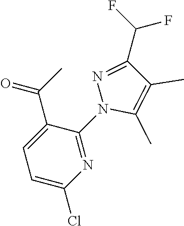 [SMILES: CC(=O)c1ccc(Cl)nc1-n1nc(C(F)F)c(C)c1C]

### Step 3: 3 formyl-4,5-dimethyl-pyrazole-1-carboxylic acid tert-butyl ester

Di-tert-butyl dicarbonate (259 mg, 1.19 mmol, 1.1 eq) was added to a suspension of 4,5-dimethyl-1H-pyrazole-3-carbaldehyde (112466-01-O) (529 mg, 4.26 mmol, 1.0 eq) in THF (10.58 mL) at 0° C. A solution of NaHCO₃ (1.07 g, 12.78 mmol, 3.000 eq) in H₂O (10 mL) was added and the reaction mixture was stirred at 0° C. for 1 hour. Then the ice bath was removed and the reaction allowed to stir at RT for 16 hours. The reaction mixture was diluted with H₂O (60 mL) and extracted with DCM (3×50 mL). The combined organic layers were dried over Na₂SO₄, filtered and concentrated. The residue was purified by flash chromatography (SiO₂, 10-50% EtOAc in heptane) to give 3-formyl-4,5-dimethyl-pyrazole-1-carboxylic acid tert-butyl ester (956 mg, 4.26 mmol, 100% yield) as a colorless oil. LC-MS: m/z=125.0 [M+H-Boc]⁺, ESI pos.

### Step 3: 3-(difluoromethyl)-4,5-dimethyl-pyrazole-1-carboxylic acid tert-butyl ester

3-formyl-4,5-dimethyl-pyrazole-1-carboxylic acid tert-butyl ester (956 mg, 4.26 mmol, 1.0 eq) was dissolved in DCM (20 mL) and cooled to −78° C. DAST (4.12 g, 3.38 mL, 25.58 mmol, 6.0 eq) was added and the reaction mixture was stirred for 1 hour at −78° C. before it was warmed to RT and stirred for 16 hours. The reaction mixture was diluted with saturated aqueous NaHCO₃ (50 mL) and extracted with DCM (3×30 mL). The combined organic layers were dried over Na₂SO₄, filtered and concentrated to give 3-(difluoromethyl)-4,5-dimethyl-pyrazole-1-carboxylic acid tert-butyl ester (1.05 g, quant. yield) which was used without further purification. LC-MS: m/z=147.0 [M+H-Boc]⁺, ESI pos.

### Step 3: 3-(difluoromethyl)-4,5-dimethyl-1H-pyrazole

3-(difluoromethyl)-4,5-dimethyl-pyrazole-1-carboxylic acid tert-butyl ester (1.05 g, 4.26 mmol, 1.0 eq) was dissolved in DCM (14 mL) and TFA (3.28 mL, 42.64 mmol, 10 eq) was added. The reaction mixture was stirred at RT for 2 hours. The solvent was removed under reduced pressure and the crude product was purified by flash chromatography (SiO₂, 10-50% EtOAc in heptane) to give 3-(difluoromethyl)-4,5-dimethyl-1H-pyrazole (381 mg, 2.60 mmol, 61% yield) as a colorless oil. LC-MS: m/z=147.0 [M+H]⁺, ESI pos.

### Step 3: 1-[6-chloro-2-[3-(difluoromethyl)-4,5-dimethyl-pyrazol-1-yl]-3-pyridyl]ethanone

To a solution of 3-(difluoromethyl)-4,5-dimethyl-1H-pyrazole (152 mg, 1.04 mmol, 1.2 eq) in DMF (2.88 mL) was added 1-(6-chloro-2-fluoro-3-pyridyl)ethanone (150 mg, 0.864 mmol, 1.0 eq) and Cs₂CO₃ (563 mg, 1.73 mmol, 2.000 eq). The reaction mixture was stirred at RT for 16 hours. The reaction mixture was diluted with H2O (50 mL) and extracted with EtOAv (3×50 mL). The combined organic layers were washed with brine (30 mL), dried over Na₂SO₄, filtered and concentrated. The residue was purified by flash chromatography (SiO₂, 0-100% EtOAc in heptane) to give 1-[6-chloro-2-[3-(difluoromethyl)-4,5-dimethyl-pyrazol-1-yl]-3-pyridyl]ethanone (118 mg, 0.328 mmol, 38%) as an off white powder. LC-MS: m/z=300.0 [M+H]⁺, ESI pos. ¹H NMR (600 MHz, DMSO-d6) δ ppm 8.17 (d, J=8.1 Hz, 1H), 7.77 (d, J=8.1 Hz, 1H), 6.89-7.09 (m, 1H), 2.43 (s, 3H), 2.17 (s, 1H), 2.12 (s, 3H), 2.10 (s, 1H), 2.01 (s, 3H)

| | | | Building | |
|---|---|---|---|---|
| Ex. | Structure | Systematic name | blocks | Analytics |
| 291 | 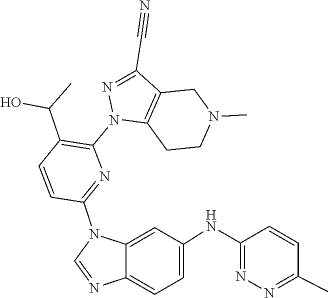 [SMILES: Cc1ccc(Nc2ccc3ncn(-c4ccc(C(C)O)c(-n5nc(C#N)c6c5CCN(C)C6)n4)c3c2)nn1] | 1-[3-(1-hydroxyethyl)- 6-[6-[(6-methylpyr- idazin-3-yl)amino]- benzimidazol-1-yl]-2- pyridyl]-5-methyl-6,7- dihydro-4~{H}-pyr- azolo[4,3-c]pyridine- 3-carbonitrile | G.1 and H.1 | LC-MS: m/z = 507.4 [M + H]+, ESI pos |
| 292 | 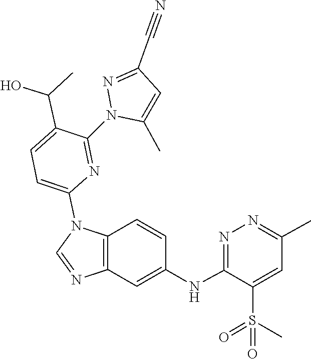 [SMILES: Cc1cc(S(C)(=O)=O)c(Nc2ccc3c(c2)ncn3-c2ccc(C(C)O)c(-n3nc(C#N)cc3C)n2)nn1] | 1-[3-(1-hydroxyethyl)- 6-[5-[(6-methyl-4- methylsulfonyl- pyridazin-3-yl)amino]- benzimidazol-1-yl]-2- pyridyl]-5-methyl- pyrazole-3-carbonitrile | G.2 and H.2 | LC-MS: m/z = 530.1 [M + H]+, ESI pos ¹H NMR (400 MHz, DMSO-d6) δ = 9.15 (s, 1H), 8.70 (s, 1H), 8.49 (d, J = 8.5 Hz, 1H), 8.31 (d, J = 8.5 Hz, 1H), 8.19 (d, J = 8.6 Hz, 1H), 7.78 (d, J = 2.0 Hz, 1H), 7.36 (dd, J = 1.9, 8.7 Hz, 1H), 7.11 (d, J = 0.6 Hz, 1H), 6.94 (s, 1H), 5.56 (d, J = 4.4 Hz, 1H), 4.61-4.50 (m, 1H), 3.53 (s, 3H), 3.30 (s, 3H), 2.45 (s, 3H), 2.34 (s, 3H), 2.07 (s, 1H), 1.26 (d, J = 6.4 Hz, 3H). |
| 295 | 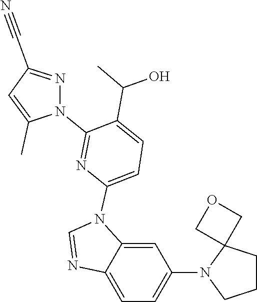 [SMILES: Cc1cc(C#N)nn1-c1nc(-n2cnc3ccc(N4CCCC45COC5)cc32)ccc1C(C)O] | 1-[3-(1-hydroxyethyl)- 6-[6-(2-oxa-5-aza- spiro[3.4]octan-5-yl)- benzimidazol-1-yl]-2- pyridyl]-5-methyl- pyrazole-3-carbonitrile | G.3 and H.2 | LC-MS: m/z = 456.4 [M + H]+, ESI pos |
| 296 | 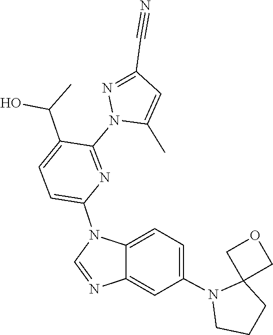 [SMILES: Cc1cc(C#N)nn1-c1nc(-n2cnc3cc(N4CCCC45COC5)ccc32)ccc1C(C)O] | 1-[3-(1-hydroxyethyl)- 6-[5-(2-oxa-5-azaspiro- [3.4]octan-5-l)benz- imidazol-1-yl]-2-pyr- idyl]-5-methyl-pyr- azole-3-carbonitrile | G.3 and H.2 | LC-MS: m/z = 456.4 [M + H]+, ESI pos |
| 301 | 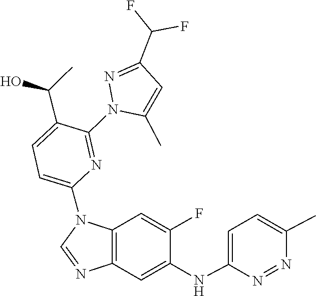 [SMILES: Cc1ccc(Nc2cc3ncn(-c4ccc([C@H](C)O)c(-n5nc(C(F)F)cc5C)n4)c3cc2F)nn1] | (1S)-1-[2-[3-(difluoro- methyl)-5-methyl- pyrazol-1-yl]-6-[6- fluoro-5-[(6-methyl- pyridazin-3-yl)amino]- benzimidazol-1-yl]-3- pyridyl]ethanol | G.18 and H.11 | LC-MS: m/z = 495.1 [M + H]+, ESI pos ¹H NMR (400 MHz, METHANOL-d4) δ ppm 8.79-8.83 (m, 1 H) 8.41-8.45 (m, 1 H) 8.35-8.39 (m, 1 H) 7.96-8.02 (m, 1 H) 7.89-7.94 (m, 1 H) 7.25-7.30 (m, 1 H) 7.06-7.12 (m, 1 H) 6.55-6.86 (m, 1 H) 6.46-6.52 (m, 1 H) 4.70-4.73 (m, 1 H) 2.42-2.45 (m, 3 H) 2.29-2.33 (m, 3 H) 1.27-1.30 (m, 3 H) |
| 310 | 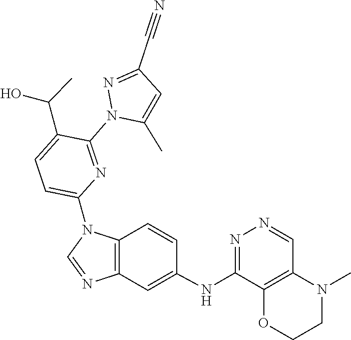 [SMILES: Cc1cc(C#N)nn1-c1nc(-n2cnc3cc(Nc4nncc5c4OCCN5C)ccc32)ccc1C(C)O] | 1-[3-(1-hydroxyethyl)- 6-[5-[(4-methyl-2,3- dihydropyridazino [4,5-b][1,4]oxazin-8- yl)amino]benzimid- azol-1-yl]-2-pyridyl]- 5-methyl-pyrazole-3- carbonitrile | G.4 and H.2 | LC-MS: m/z = 509.1 [M + H]+, ESI pos ¹H NMR (400 MHz, acetonitrile-d3) δ = 8.60 (s, 1H), 8.41 (d, J = 8.5 Hz, 1H), 8.30 (s, 1H), 8.22 (d, J = 2.0 Hz, 1H), 8.11 (s, 1H), 7.99 (d, J = 8.9 Hz, 1H), 7.94 (d, J = 8.5 Hz, 1H), 7.55 (dd, J = 2.1, 8.9 Hz, 1H), 7.17 (br s, 1H), 6.81 (s, 1H), 4.70 (q, J = 6.5 Hz, 1H), 4.44-4.36 (m, 2H), 3.44-3.38 (m, 2H), 2.99 (s, 3H), 2.35 (s, 3H), 1.34 (d, J = 6.5 Hz, 3H) |
| 312 | 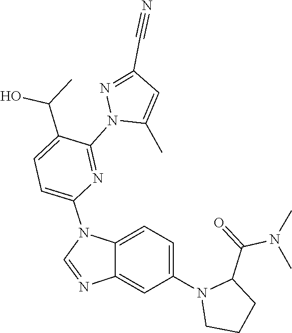 [SMILES: Cc1cc(C#N)nn1-c1nc(-n2cnc3cc(N4CCCC4C(=O)N(C)C)ccc32)ccc1C(C)O] | 1-[1-[6-(3-cyano-5- methyl-pyrazol-1-yl)- 5-(1-hydroxyethyl)-2- pyridyl]benzimidazol- 5-yl]-N,N-dimethyl- pyrrolidine-2-carbox- amide | G.5 and H.2 | LC-MS: m/z = 485.3 [M + H]+, ESI pos |
| 318 | 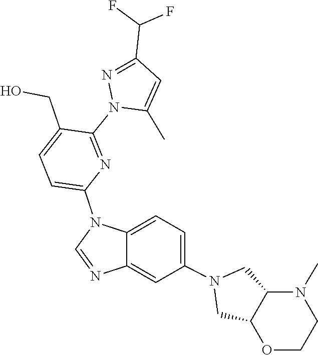 [SMILES: Cc1cc(C(F)F)nn1-c1nc(-n2cnc3cc(N4C[C@H]5OCCN(C)[C@H]5C4)ccc32)ccc1CO] | [6-[5-[(4aS,7aR)-4- methyl-2,3,4a,5,7,7a- hexahydropyrrolo[3,4- b][1,4]oxazin-6-yl]- benzimidazol-1-yl]-2- [3-(difluoromethyl)-5- methyl-pyrazol-1-yl]- 3-pyridyl]methanol | G.6 and H.3 followed by reduction in analogy to Example 1, step 3 | LC-MS: m/z = 496.4 [M + H]+, ESI pos |
| 327 | 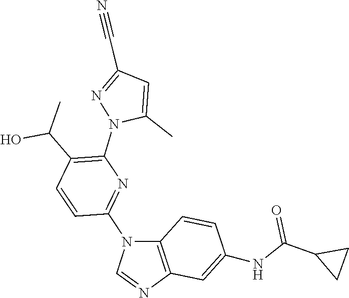 [SMILES: Cc1cc(C#N)nn1-c1nc(-n2cnc3cc(NC(=O)C4CC4)ccc32)ccc1C(C)O] | N-[1-[6-(3-cyano-5- methyl-pyrazol-1-yl)- 5-(1-hydroxyethyl)-2- pyridyl]benzimidazol- 5-yl]cyclopropane- carboxamide | G.7 and H.2 | LC-MS: m/z = 428.0 [M + H]⁺, ESI pos ¹H NMR (400 MHz, METHANOL-d4) δ = 8.92 (s, 1H), 8.51 (d, J = 8.6 Hz, 1H), 8.16- 8.13 (m, 2H), 8.10 (d, J = 8.9 Hz, 1H), 7.52 (dd, J = 1.7, 8.9 Hz, 1H), 6.88 (s, 1H), 4.78 (q, J = 6.5 Hz, 1H), 2.41 (s, 3H), 1.86-1.78 (m, 1H), 1.43 (d, J = 6.4 Hz, 3H), 1.03-0.97 (m, 2H), 0.92-0.86 (m, 2H) |
| 328 | 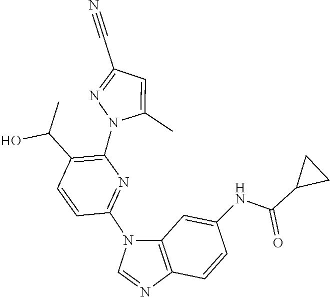 [SMILES: Cc1cc(C#N)nn1-c1nc(-n2cnc3ccc(NC(=O)C4CC4)cc32)ccc1C(C)O] | N-[3-[6-(3-cyano-5- methyl-pyrazol-1-yl)- 5-(1-hydroxyethyl)-2- pyridyl]benzimidazol- 5-yl]cyclopropane- carboxamide | G.7 and H.2 | LC-MS: m/z = 428.0 [M + H]+, ESI pos ¹H NMR (400 MHz, METHANOL-d4) δ = 8.84 (s, 1H), 8.60 (d, J = 1.3 Hz, 1H), 8.52 (d, J = 8.6 Hz, 1H), 8.11 (d, J = 8.6 Hz, 1H), 7.69 (d, J = 8.7 Hz, 1H), 7.45 (dd, J = 1.8, 8.7 Hz, 1H), 6.86 (s, 1H), 4.78 (q, J = 6.3 Hz, 1H), 2.44 (s, 3H), 1.86-1.75 (m, 1H), 1.43 (d, J = 6.5 Hz, 3H), 1.00-0.95 (m, 2H), 0.91-0.85 (m, 2H) |
| 330 | 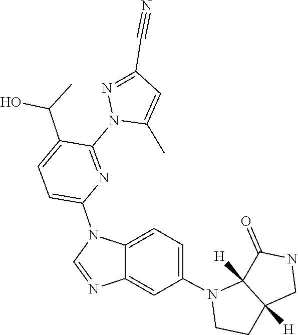 | 1-[6-[5-[(3aS,6aS)-6- oxo-2,3,3a,4,5,6a- hexahydropyrrolo[2,3- c]pyrrol-1-yl]benz- imidazol-1-yl]-3-(1- hydroxyethyl)-2-pyr- idyl]-5-methylpyr- azole-3-carbonitrile | G.8 and H.2 | LC-MS: m/z = 469.3 [M + H]+, ESI pos |
| 334 | 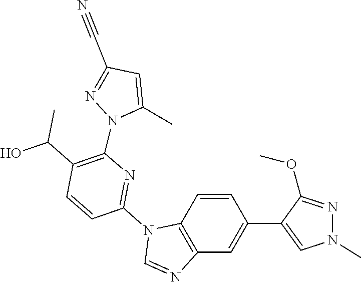 [SMILES: COc1nn(C)cc1-c1ccc2c(c1)ncn2-c1ccc(C(C)O)c(-n2nc(C#N)cc2C)n1] | 1-[3-(1-hydroxyethyl)- 6-[5-(3-methoxy-1- methyl-pyrazol-4-yl)- benzimidazol-1-yl]-2- pyridyl]-5-methyl- pyrazole-3-carbonitrile | G.21 and H.2 | LC-MS: m/z = 455.5 [M + H]+, ESI pos ¹H NMR (300 MHz, DMSO-d6) δ = 9.02 (s, 1H), 8.46 (d, J = 8.5 Hz, 1H), 8.27 (d, J = 8.5 Hz, 1H), 8.13- 7.95 (m, 3H), 7.63 (dd, J = 1.6, 8.7 Hz, 1H), 7.10 (d, J = 0.8 Hz, 1H), 5.55 (d, J = 4.2 Hz, 1H), 4.68- 4.50 (m, 1H), 3.93 (s, 3H), 3.73 (s, 3H), 2.35 (d, J = 0.6 Hz, 3H), 1.27 (d, J = 6.4 Hz, 3H) |
| 336 | 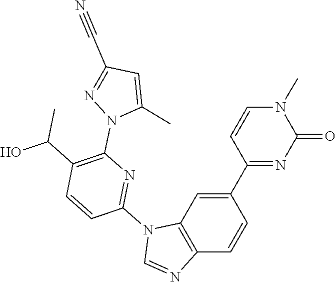 [SMILES: Cc1cc(C#N)nn1-c1nc(-n2cnc3ccc(-c4ccn(C)c(=O)n4)cc32)ccc1C(C)O] | 1-[3-(1-hydroxyethyl)- 6-[6-(1-methyl-2-oxo- pyrimidin-4-yl)benz- imidazol-1-yl]-2-pyr- idyl]-5-methyl-pyr- azole-3-carbonitrile | G.9 and H.2 | LC-MS: m/z = 453.3 [M + H]+, ESI pos ¹H NMR (400 MHz, METHANOL-d4) δ = 9.03 (s, 1H), 8.97 (d, J = 1.2 Hz, 1H), 8.56 (d, J = 8.4 Hz, 1H), 8.20 (dd, J = 7.6, 11.9 Hz, 2H), 8.14 (dd, J = 1.6, 8.6 Hz, 1H), 7.88 (d, J = 8.6 Hz, 1H), 7.08 (d, J = 6.8 Hz, 1H), 6.87 (s, 1H), 4.84 (br d, J = 6.6 Hz, 1H), 3.62 (s, 3H), 2.49 (s, 3H), 1.43 (d, J = 6.5 Hz, 3H) |
| 338 | 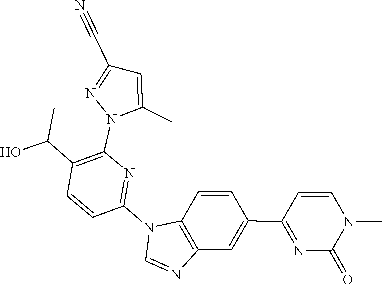 | 1-[3-(1-hydroxyethyl)- 6-[5-(1-methyl-2-oxo- pyrimidin-4-yl)benz- imidazol-1-yl]-2-pyr- idyl]-5-methyl-pyr- azole-3-carbonitrile | G.9 and H.2 | LC-MS: m/z = 567.2 [M + H]+, ESI pos ¹H NMR (400 MHz, METHANOL-d4) δ = 8.95 (s, 1H), 8.49 (dd, J = 4.0, 7.9 Hz, 2H), 8.12 (d, J = 8.3 Hz, 1H), 8.06 (d, J = 11.1 Hz, 1H), 7.62 (d, J = 9.3 Hz, 1H), 7.23 (d, J = 9.3 Hz, 1H), 6.99- 6.65 (m, 1H), 6.60 (s, 1H), 4.83 (d, J = 6.6 Hz, 1H), 4.13-4.08 (m, 2H), 3.93-3.89 (m, 2H), 2.41 (s, 3H), 1.72 (s, 3H), 1.39 (d, J = 6.5 Hz, 3H) |
| 339 | 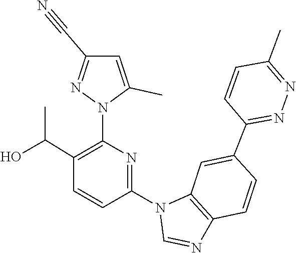 [SMILES: Cc1ccc(-c2ccc3ncn(-c4ccc(C(C)O)c(-n5nc(C#N)cc5C)n4)c3c2)nn1] | 1-[3-(1-hydroxyethyl)- 6-[6-(6-methylpyr- idazin-3-yl)benzimid- azol-1-yl]-2-pyridyl]- 5-methyl-pyrazole-3- carbonitrile | G.10 and H.2 | LC-MS: m/z = 437.2 [M + H]+, ESI pos ¹H NMR (400 MHz, METHANOL-d4) δ = 9.02 (s, 1H), 8.94 (d, J = 1.1 Hz, 1H), 8.55 (d, J = 8.4 Hz, 1H), 8.22 (d, J = 8.4 Hz, 1H), 8.09 (d, J = 8.8 Hz, 1H), 8.05 (dd, J = 1.7, 8.5 Hz, 1H), 7.92 (d, J = 8.6 Hz, 1H), 7.69 (d, J= 8.8 Hz, 1H), 6.87 (d, J = 0.7 Hz, 1H), 4.82 (d, J = 6.5 Hz, 1H), 2.74 (s, |
| | | | | 3H), 2.47 (s, 3H), 1.42 |
| | | | | (d, J = 6.5 Hz, 3H) |
| 340 | 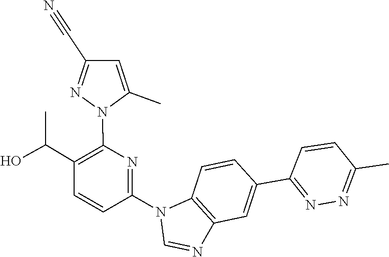 [SMILES: Cc1ccc(-c2ccc3c(c2)ncn3-c2ccc(C(C)O)c(-n3nc(C#N)cc3C)n2)nn1] | 1-[3-(1-hydroxyethyl)- 6-[5-(6-methylpyr- idazin-3-yl)benzimid- azol-1-yl]-2-pyridyl]- 5-methyl-pyrazole-3- carbonitrile | G.10 and H.2 | LC-MS: m/z = 437.1 [M + H]+, ESI pos ¹H NMR (400 MHz, METHANOL-d4) δ = 9.02 (s, 1H), 8.54 (d, J = 8.4 Hz, 1H), 8.47 (s, 1H), 8.33 (d, J = 8.7 Hz, 1H), 8.22- 8.13 (m, 3H), 7.70 (d, J = 8.8 Hz, 1H), 6.88 (s, 1H), 4.81-4.77 (m, 1H), 2.74 (s, 3H), 2.42 (s, 3H), 1.42 (d, J = 6.5 Hz, 3H) |
| 341 | 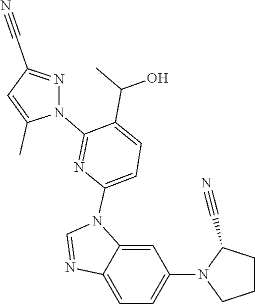 [SMILES: Cc1cc(C#N)nn1-c1nc(-n2cnc3ccc(N4CCC[C@H]4C#N)cc32)ccc1C(C)O] | 1-[6-[6-[(2S)-2-cyano- pyrrolidin-1-yl]benz- imidazol-1-yl]-3-(1- hydroxyethyl)-2-pyr- idyl]-5-methyl-pyr- azole-3-carbonitrile | G.11 and H.2 | LC-MS: m/z = 439.32 [M + H]+, ESI pos |
| 342 | 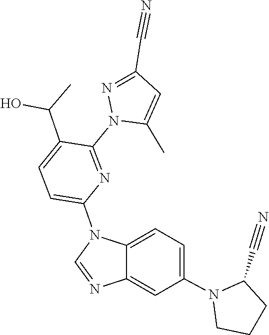 [SMILES: Cc1cc(C#N)nn1-c1nc(-n2cnc3cc(N4CCC[C@H]4C#N)ccc32)ccc1C(C)O] | 1-[6-[5-[(2S)-2-cyano- pyrrolidin-1-yl]benz- imidazol-1-yl]-3-(1- hydroxyethyl)-2-pyr- idyl]-5-methyl-pyr- azole-3-carbonitrile | G.11 and H.2 | LC-MS: m/z = 439.32 [M + H]+, ESI pos |
| 343 | 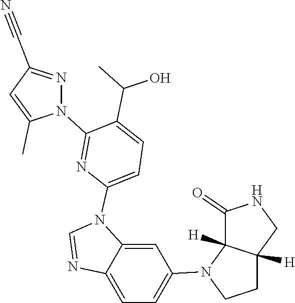 [SMILES: Cc1cc(C#N)nn1-c1nc(-n2cnc3ccc(N4CC[C@H]5CNC(=O)[C@H]54)cc32)ccc1C(C)O] | 1-[6-[6-[(3aS,6aS)-6- oxo-2,3,3a,4,5,6a- hexahydropyrrolo[2,3- c]pyrrol-1-yl]benz- imidazol-1-yl]-3-(1- hydroxyethyl)-2-pyr- idyl]-5-methyl-pyr- azole-3-carbonitrile | G.8 and H.2 | LC-MS: m/z = 469.32 [M + H]+, ESI pos |
| 350 | 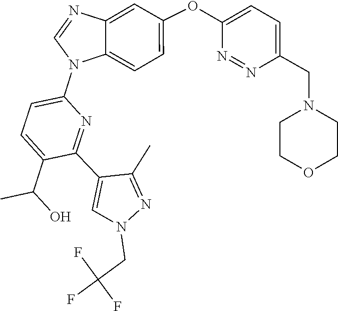 [SMILES: Cc1nn(CC(F)(F)F)cc1-c1nc(-n2cnc3cc(Oc4ccc(CN5CCOCC5)nn4)ccc32)ccc1C(C)O] | 1-[2-[3-methyl-1- (2,2,2-trifluoroethyl)- pyrazol-4-yl]-6-[5-[6- (morpholinomethyl)- pyridazin-3-yl]oxy- benzimidazol-1-yl]-3- pyridyl]ethanol | G.12 and H.4 | LC-MS: m/z = 595.3 [M + H]+, ESI pos ¹H NMR (400 MHz, METHANOL-d4) δ = 9.00-8.88 (m, 1H), 8.40-8.23 (m, 2H), 8.02 (s, 1H), 7.92- 7.79 (m, 2H), 7.59 (d, J = 2.0 Hz, 1H), 7.48- 7.37 (m, 1H), 7.34- 7.18 (m, 1H), 5.06- 4.95 (m, 3H), 3.80 (s, 2H), 3.77-3.61 (m, 4H), 2.54 (s, 4H), 2.42-2.28 (m, 3H), 1.48 (d, J = 6.4 Hz, 3H) |
| 351 | 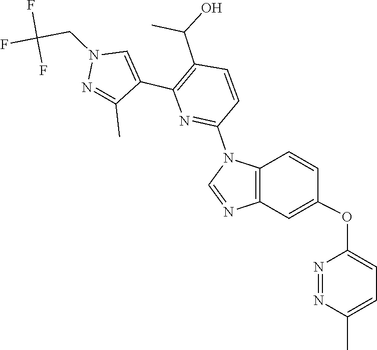 [SMILES: Cc1ccc(Oc2ccc3c(c2)ncn3-c2ccc(C(C)O)c(-c3cn(CC(F)(F)F)nc3C)n2)nn1] | 1-[6-[5-(6-methylpyr- idazin-3-yl)oxybenz- imidazol-1-yl]-2-[3- methyl-1-(2,2,2-tri- fluoroethyl)pyrazol-4- yl]-3-pyridyl]ethanol | G.13 and H.4 | LC-MS: m/z = 510.2 [M + H]+, ESI pos ¹H NMR (400 MHz, METHANOL-d4) δ = 8.93 (s, 1H), 8.30 (t, J = 9.1 Hz, 2H), 8.02 (s, 1H), 7.87 (d, J = 8.4 Hz, 1H), 7.64 (d, J = 9.0 Hz, 1H), 7.57 (d, J = 2.1 Hz, 1H), 7.34 (d, J = 9.0 Hz, 1H), 7.21 (br d, J = 2.9 Hz, 1H), 5.12-4.93 (m, 3H), 2.61 (s, 3H), 2.35 (s, 3H), 1.47 (d, J = 6.5 Hz, 3H) |
| 352 | 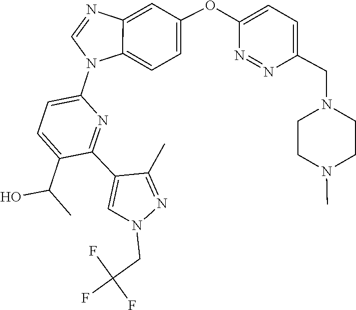 [SMILES: Cc1nn(CC(F)(F)F)cc1-c1nc(-n2cnc3cc(Oc4ccc(CN5CCN(C)CC5)nn4)ccc32)ccc1C(C)O] | 1-[6-[5-[6-[(4-methyl- piperazin-1-yl)meth- yl]pyridazin-3-yl]oxy- benzimidazol-1-yl]-2- [3-methyl-1-(2,2,2- trifluoroethyl)pyrazol- 4-yl]-3-pyridyl]ethanol | G.14 and H.4 | LC-MS: m/z = 608.3 [M + H]+, ESI pos ¹H NMR (400 MHz, METHANOL-d4) δ = 9.36-9.28 (m, 1H), 8.41-8.32 (m, 2H), 8.07 (s, 1H), 8.04 (s, 1H), 7.97-7.84 (m, 2H), 7.68 (s, 1H), 7.51 (d, J = 9.2 Hz, 1H), 7.38 (d, J = 9.2 Hz, 1H), 5.08-4.96 (m, 3H), 3.92 (s, 2H), 3.60-3.40 (m, 2H), 3.28-2.97 (m, 4H), 2.90 (s, 3H), 2.80- 2.46 (m, 2H), 2.35 (s, 3H), 1.48 (d, J = 6.4 Hz, 3H) |
| 358 | 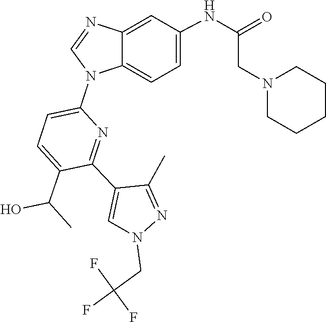 [SMILES: Cc1nn(CC(F)(F)F)cc1-c1nc(-n2cnc3cc(NC(=O)CN4CCCCC4)ccc32)ccc1C(C)O] | N-[1-[5-(1-hydroxy- ethyl)-6-[3-methyl-1- (2,2,2-trifluoroethyl)- pyrazol-4-yl]-2-pyr- idyl]benzimidazol-5- yl]-2-(1-piperidyl)- acetamide | G.15 and H.4 | LC-MS: m/z = 542.3 [M + H]+, ESI pos ¹H NMR (400 MHz, METHANOL-d4) δ = 8.88 (s, 1H), 8.30 (d, J = 8.5 Hz, 1H), 8.23- 8.15 (m, 2H), 8.02 (s, 1H), 7.84 (br d, J = 7.0 Hz, 1H), 7.50 (br d, J = 8.8 Hz, 1H), 5.03-4.96 (m, 3H), 4.59 (s, 2H), 3.67 (br s, 2H), 3.01 (br s, 2H), 2.34 (s, 3H), 1.86-1.79 (m, 4H), 1.63 (br d, J = 4.3 Hz, 2H), 1.47 (d, J = 6.5 Hz, 3H) |
| 365 | 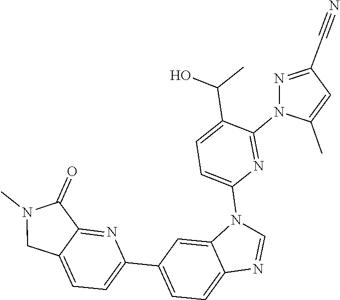 [SMILES: Cc1cc(C#N)nn1-c1nc(-n2cnc3ccc(-c4ccc5c(n4)C(=O)N(C)C5)cc32)ccc1C(C)O] | 1-[3-(1-hydroxyethyl)- 6-[6-(6-methyl-7-oxo- 5H-pyrrolo[3,4-b]- pyridin-2-yl)benzimid- azol-1-yl]-2-pyridyl]- 5-methyl-pyrazole-3- carbonitrile | G.16 and H.2 | LC-MS: m/z = 491.2 [M + H]+, ESI pos ¹H NMR (400 MHz, DMSO-d6) δ = 9.04 (s, 1H), 8.92 (d, J = 1.4 Hz, 1H), 8.44 (d, J = 8.4 Hz, 1H), 8.25 (d, J = 8.4 Hz, 1H), 8.07-8.03 (m, 3H), 7.82 (d, J = 8.5 Hz, 1H), 7.00 (d, J = 0.6 Hz, 1H), 5.82-5.28 (brs, 1H), 4.59 (q, J = 6.3 Hz, 1H), 4.45 (s, 2H), 3.10 (s, 3H), 2.51 (s, 3H), 1.22 (d, J = 6.5 Hz, 3H) |
| 366 | 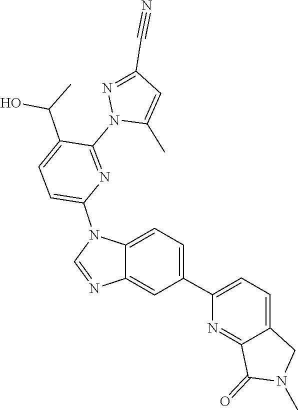 [SMILES: Cc1cc(C#N)nn1-c1nc(-n2cnc3cc(-c4ccc5c(n4)C(=O)N(C)C5)ccc32)ccc1C(C)O] | 1-[3-(1-hydroxyethyl)- 6-[5-(6-methyl-7-oxo- 5H-pyrrolo[3,4-b]- pyridin-2-yl)benzimid- azol-1-yl]-2-pyridyl]- 5-methyl-pyrazole-3- carbonitrile | G.16 and H.2 | LC-MS: m/z = 491.2 [M + H]+, ESI pos ¹H NMR (400 MHz, DMSO-d6) δ = 9.11 (s, 1H), 8.53 (s, 1H), 8.48 (d, J = 8.5 Hz, 1H), 8.30 (d, J = 8.5 Hz, 1H), 8.24-8.17 (m, 3H), 8.13-8.08 (m, 1H), 7.09 (d, J = 0.8 Hz, 1H), 5.53 (br d, J = 2.9 Hz, 1H), 4.58 (br d, J = 6.4 Hz, 1H), 4.50 (s, 2H), 3.13 (s, 3H), 2.36 (s, 3H), 1.25 (d, J = 6.4 Hz, 3H) |
| 368 | 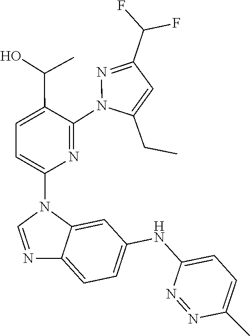 [SMILES: CCc1cc(C(F)F)nn1-c1nc(-n2cnc3ccc(Nc4ccc(C)nn4)cc32)ccc1C(C)O] | 1-[2-[3-(difluorometh- yl)-5-ethyl-pyrazol-1- yl]-6-[6-[(6-methyl- pyridazin-3-yl)amino]- benzimidazol-1-yl]-3- pyridyl]ethanol | G.1 and H.5 | LC-MS: m/z = 491.2 [M + H]+, ESI pos ¹H NMR (600 MHz, DMSO-d6) δ ppm 9.26 (s, 1 H), 8.82 (s, 1 H), 8.69 (d, J = 2.0 Hz, 1 H), 8.46 (d, J = 8.4 Hz, 1 H), 8.15 (d, J = 8.4 Hz, 1 H), 7.69 (d, J = 8.7 Hz, 1 H), 7.42 (dd, J = 8.7, 2.1 Hz, 1 H), 7.27 (d, J = 9.1 Hz, 1 H), 7.06 (d, J = 9.1 Hz, 1 H), 6.94- 7.14 (m, 1H), 6.62 (s, 1 H), 5.50 (d, J =4.3 Hz, 1 H), 4.60-4.64 (m, 1 H), 2.72 (q, J = .5 Hz, 2 H), 2.47-2.48 (m, 3 H), 1.24 (d, J = 6.3 Hz, 5 H), 1.10 (t, |
| | | | | J =7.6 Hz, 3 H) |
| 369 | 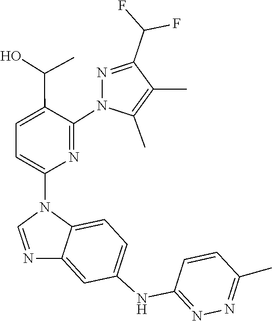 [SMILES: Cc1ccc(Nc2ccc3c(c2)ncn3-c2ccc(C(C)O)c(-n3nc(C(F)F)c(C)c3C)n2)nn1] | 1-[2-[3-(difluorometh- yl)-4,5-dimethyl-pyr- azol-1-yl]-6-[5-[(6- methylpyridazin-3-yl)- amino]benzimidazol- 1-yl]-3-pyridyl]ethanol | G.1 and H.12 | LC-MS: m/z = 491.2 [M + H]+, ESI pos ¹H NMR (600 MHz, DMSO-d6) δ ppm 9.20 (s, 1 H), 8.98 (s, 1 H), 8.41 (d, J = 8.1 Hz, 1 H), 8.40 (d, J = 2.1 Hz, 1 H), 8.19 (d, J = 8.2 Hz, 1 H), 8.06 (d, J = 9.0 Hz, 1 H), 7.52 (dd, J = 8.8, 2.1 Hz, 1 H), 7.33 (d, J = 9.1 Hz, 1 H), 7.08 (d, J = 9.1 Hz, 1 H), 6.95-7.15 (m, 2 H), 5.43-5.46 (m, 1 H), 4.63-4.68 (m, 1 H), 2.48 (s, 4 H), 2.26 (s, 3 H), 2.17 (s, 3 H), 1.23 (d, J = 6.4 Hz, 4 H) |
| 370 | 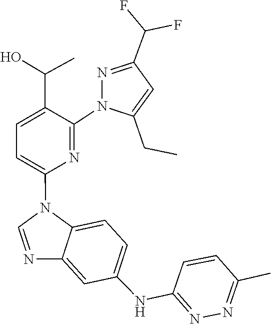 [SMILES: CCc1cc(C(F)F)nn1-c1nc(-n2cnc3cc(Nc4ccc(C)nn4)ccc32)ccc1C(C)O] | 1-[2-[3-(difluorometh- yl)-5-ethyl-pyrazol-1- yl]-6-[5-[(6-methyl- pyridazin-3-yl)amino]- benzimidazol-1-yl]-3- pyridyl]ethanol | G.1 and H.5 | LC-MS: m/z = 491.2 [M + H]+, ESI pos ¹H NMR (600 MHz, DMSO-d6) δ ppm 9.20 (s, 1 H), 8.99 (s, 1 H), 8.42-8.44 (m, 1 H), 8.42-8.43 (m, 1 H), 8.22 (d, J = 8.4 Hz, 1 H), 8.05 (d, J = 8.9 Hz, 1 H), 7.48 (dd, J = 8.9, 2.1 Hz, 1 H), 7.33 (d, J = 9.2 Hz, 1 H), 7.08 (d, J = 8.7 Hz, 1 H), 6.98 (s, 1 H), 6.69 (s, 1 H), 5.48 (d, J = 4.5 Hz, 1 H), 4.59-4.67 (m, 1 H), 2.68-2.74 (m, 2 H), 2.48 (s, 3 H), 1.23 (d, J = 6.4 Hz, 4 H), 1.15- |
| | | | | 1.19 (m, 3 H) |
| 380 | 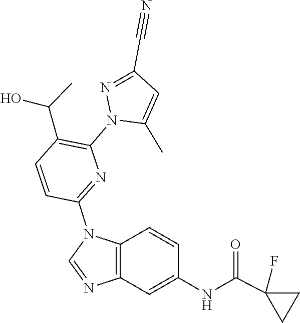 [SMILES: Cc1cc(C#N)nn1-c1nc(-n2cnc3cc(NC(=O)C4(F)CC4)ccc32)ccc1C(C)O] | N-[1-[6-(3-cyano-5- methyl-pyrazol-1-yl)- 5-(1-hydroxyethyl)-2- pyridyl]benzimidazol- 5-yl]-1-fluoro-cyclo- propanecarboxamide | G.17 and H.2 | LC-MS: m/z = 446.2 [M + H]+, ESI pos ¹H NMR (400 MHz, DMSO-d6) δ = 10.35 (d, J = 1.7 Hz, 1H), 9.05 (s, 1H), 8.47 (d, J = 8.4 Hz, 1H), 8.28 (d, J = 8.6 Hz, 1H), 8.19 (d, J = 1.8 Hz, 1H), 8.06 (d, J = 8.8 Hz, 1H), 7.69 (dd, J = 2.0, 8.9 Hz, 1H), 7.10 (s, 1H), 5.63-5.49 (m, 1H), 4.58 (q, J = 6.3 Hz, 1H), 2.35 (s, 3H), 1.48-1.44 (m, 1H), 1.42-1.39 (m, 1H), 1.38-1.33 (m, 2H), 1.27 (d, J = 6.5 Hz, 3H) |
| 387 | 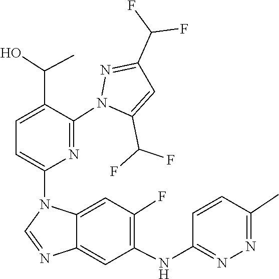 [SMILES: Cc1ccc(Nc2cc3ncn(-c4ccc(C(C)O)c(-n5nc(C(F)F)cc5C(F)F)n4)c3cc2F)nn1] | 1-[2-[3,5-bis(difluoro- methyl)pyrazol-1-yl]- 6-[6-fluoro-5-[(6- methylpyridazin-3-yl)- amino]benzimidazol- 1-yl]-3-pyridyl]ethanol | G.18 and H.6 | LC-MS: m/z = 531.1 [M + H]⁺, ESI pos ¹H NMR (600 MHz, DMSO-d6) d ppm 9.06 (s, 1 H), 8.85 (s, 1 H), 8.66 (d, J = 7.8 Hz, 1 H), 8.48 (d, J = 8.2 Hz, 1 H), 8.28 (d, J = 8.3 Hz, 1 H), 8.02 (d, J = 11.6 Hz, 1 H), 7.37-7.49 (m, 1 H), 7.35 (d, J = 9.1 Hz, 1 H), 7.33 (s, 1 H), 7.31 (s, 1 H), 7.30 (s, 1 H), 7.24 (d, J = 9.1 Hz, 1 H), 7.12-7.23 (m, 1 H), 5.56 (d, J = 4.6 Hz, 1 H), 4.77-4.88 |
| | | | | (m, 1 H), 1.26 (d, J = |
| | | | | 6.3 Hz, 3 H) |
| 392 | 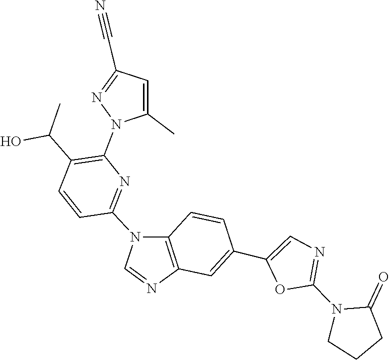 [SMILES: Cc1cc(C#N)nn1-c1nc(-n2cnc3cc(-c4cnc(N5CCCC5=O)o4)ccc32)ccc1C(C)O] | 1-[3-(1-hydroxyethyl)- 6-[5-[2-(2-oxopyr- rolidin-1-yl)oxazol-5- yl]benzimidazol-1-yl]- 2-pyridyl]-5-methyl- pyrazole-3-carbonitrile | G.19 and H.2 | LC-MS: m/z = 495.1 [M + H]+, ESI pos ¹H NMR (400 MHz, METHANOL-d4) δ = 8.97 (s, 1H), 8.52 (d, J = 8.5 Hz, 1H), 8.23 (d, J = 8.6 Hz, 1H), 8.16 (d, J = 8.4 Hz, 1H), 8.06 (d, J = 1.1 Hz, 1H), 7.75 (dd, J = 1.6, 8.7 Hz, 1H), 7.45 (s, 1H), 6.87 (s, 1H), 4.78 (q, J = 6.5 Hz, 1H), 4.09 (t, J = 7.1 Hz, 2H), 2.69-2.62 (m, 2H), 2.41 (s, 3H), 2.26 (t, J = 7.6 Hz, 2H), 1.41 (d, J = 6.4 Hz, 3H). |
| 395 | 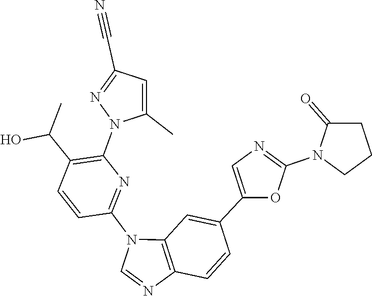 [SMILES: Cc1cc(C#N)nn1-c1nc(-n2cnc3ccc(-c4cnc(N5CCCC5=O)o4)cc32)ccc1C(C)O] | 1-[3-(1-hydroxyethyl)- 6-[5-[2-(2-oxopyr- rolidin-1-yl)oxazol-5- yl]benzimidazol-1-yl]- 2-pyridyl]-5-methyl- pyrazole-3-carbonitrile | G.19 and H.2 | LC-MS: m/z = 495.1 [M + H]+, ESI pos ¹H NMR (400 MHz, DMSO-d6) δ = 9.11 (s, 1H), 8.49 (d, J = 8.5 Hz, 1H), 8.39 (s, 1H), 8.31 (d, J = 8.5 Hz, 1H), 7.86 (d, J = 8.5 Hz, 1H), 7.69 (dd, J = 1.4, 8.4 Hz, 1H), 7.55 (s, 1H), 7.07 (s, 1H), 5.56 (br d, J = 4.3 Hz, 1H), 4.66- 4.53 (m, 1H), 3.89 (t, J = 7.1 Hz, 2H), 2.59- 2.57 (m, 2H), 2.39 (s, 3H), 2.19-2.11 (m, 2H), 1.28 (d, J = 6.4 Hz, 3H) |
| 397 | 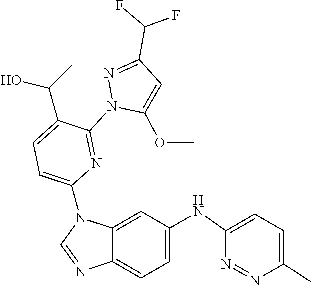 [SMILES: COc1cc(C(F)F)nn1-c1nc(-n2cnc3ccc(Nc4ccc(C)nn4)cc32)ccc1C(C)O] | 1-[2-[3-(difluorometh- yl)-5-methoxy-pyrazol- 1-yl]-6-[6-[(6-methyl- pyridazin-3-yl)amino]- benzimidazol-1-yl]-3- pyridyl]ethanol | G.1 and H.7 | LC-MS: m/z = 493.4 [M + H]+, ESI pos ¹H NMR (400 MHz, METHANOL-d4) δ = 8.88-8.70 (m, 2H), 8.58-8.46 (m, 1H), 8.18-8.04 (m, 1H), 7.78-7.67 (m, 1H), 7.48-7.30 (m, 2H), 7.24-7.10 (m, 1H), 6.94-6.47 (m, 1H), 6.23-6.04 (m, 1H), 4.96-4.94 (m, 1H), 3.99-3.85 (m, 3H), 2.62-2.49 (m, 3H), 1.45-1.34 (m, 3H) |
| 398 | 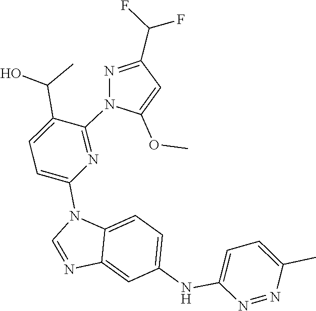 [SMILES: COc1cc(C(F)F)nn1-c1nc(-n2cnc3cc(Nc4ccc(C)nn4)ccc32)ccc1C(C)O] | 1-[2-[3-(difluorometh- yl)-5-methoxy-pyrazol- 1-yl]-6-[5-[(6-methyl- pyridazin-3-yl)amino]- benzimidazol-1-yl]-3- pyridyl]ethanol | G.1 and H.7 | LC-MS: m/z = 493.4 [M + H]⁺, ESI pos ¹H NMR (400 MHz, METHANOL-d4) δ = 8.98-8.81 (m, 1H), 8.31 (br dd, J = 1.8, 3.2 Hz, 1H), 8.29-8.16 (m, 2H), 8.14-7.98 (m, 1H), 7.71-7.52 (m, 1H), 7.50-7.30 (m, 1H), 7.26-7.03 (m, 1H), 6.98-6.49 (m, 1H), 6.42-6.02 (m, 1H), 4.91 (br s, 1H), 4.28-3.82 (m, 3H), 2.63-2.42 (m, 3H), 1.48-1.31(m, 3H) |
| 401 | 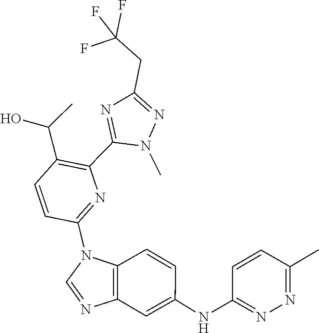 [SMILES: Cc1ccc(Nc2ccc3c(c2)ncn3-c2ccc(C(C)O)c(-c3nc(CC(F)(F)F)nn3C)n2)nn1] | 1-[6-[5-[(6-methyl- pyridazin-3-yl)amino]- benzimidazol-1-yl]-2- [2-methyl-5-(2,2,2- trifluoroethyl)-1,2,4- triazol-3-yl]-3-pyr- idyl]ethanol | G.1 and H.8 | LC-MS: m/z = 510.1 [M + H]+, ESI pos ¹H NMR (400 MHz, METHANOL-d4) δ = 8.92 (s, 1H), 8.46 (d, J = 8.7 Hz, 1H), 8.25 (d, J = 2.0 Hz, 1H), 8.12 (dd, J = 8.8, 14.7 Hz, 2H), 7.65 (dd, J = 1.8, 8.9 Hz, 1H), 7.39 (d, J = 9.2 Hz, 1H), 7.16 (d, J = 9.2 Hz, 1H), 5.31-5.26 (m, 1H), 4.09 (s, 3H), 3.83-3.73 (m, 2H), 2.55 (s, 3H), 1.52 (d, J = 6.5 Hz, 3H) |
| 404 | 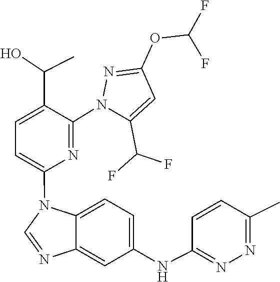 [SMILES: Cc1ccc(Nc2ccc3c(c2)ncn3-c2ccc(C(C)O)c(-n3nc(OC(F)F)cc3C(F)F)n2)nn1] | 1-[2-[3-(difluorometh- oxy)-5-(difluorometh- yl)pyrazol-1-yl]-6-[5- [(6-methylpyridazin- 3-yl)amino]benzimid- azol-1-yl]-3-pyridyl]- ethanol | G.1 and H.9 | LC-MS: m/z = 529.2 [M + H]+, ESI pos ¹H NMR (600 MHz, DMSO-d6) δ ppm 9.25-9.28 (m, 1 H), 8.96 (s, 1 H), 8.45 (d, J = 8.4 Hz, 1 H), 8.42 (d, J = 2.0 Hz, 1 H), 8.17 (d, J = 8.3 Hz, 1 H), 8.05 (d, J = 8.9 Hz, 1 H), 7.49 (dd, J = 8.9, 2.1 Hz, 1 H), 7.45 (s, 1 H), 7.38 (s, 1 H), 7.35 (br s, 1 H), 7.33- 7.37 (m, 1 H), 7.11- 7.14 (m, 1 H), 6.86 (s, 1 H), 5.51 (d, J = 4.6 Hz, 1 H), 4.97 (dd, J = 6.3, 4.5 Hz, 1 H), 2.47- 2.48 (m, 2 H), 1.28- 1.32 (m, 3 H) |
| 405 | 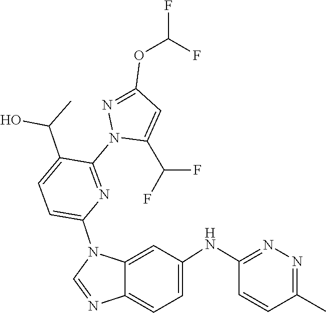 [SMILES: Cc1ccc(Nc2ccc3ncn(-c4ccc(C(C)O)c(-n5nc(OC(F)F)cc5C(F)F)n4)c3c2)nn1] | 1-[2-[3-(difluorometh- oxy)-5-difluorometh- yl)pyrazol-1-yl]-6-[6- [(6-methylpyridazin- 3-yl)amino]benzimid- azol-1-yl]-3-pyridyl]- ethanol | G.1 and H.9 | LC-MS: m/z = 529.2 [M + H]+, ESI pos ¹H NMR (600 MHz, DMSO-d6) d ppm 9.33 (br s, 1 H), 8.89 (s, 1 H), 8.85 (s, 1 H), 8.47 (d, J = 8.4 Hz, 1 H), 8.12 (d, J = 8.4 Hz, 1 H), 7.70 (d, J = 8.7 Hz, 1 H), 7.53- 7.56 (m, 1 H), 7.45- 7.46 (m, 1 H), 7.39 (dd, J = 8.7, 2.1 Hz, 1 H), 7.30-7.35 (m, 1 H), 7.05-7.14 (m, 1 H), 6.78 (s, 1 H), 5.53 (br d, J = 4.1 Hz, 1 H), 4.98-5.06 (m, 1 H), 2.48 (s, 2 H), 2.43- |
| | | | | 2.48 (m, 1 H), 1.31 (d, |
| | | | | J = 6.3 Hz, 3 H) |
| 406 | 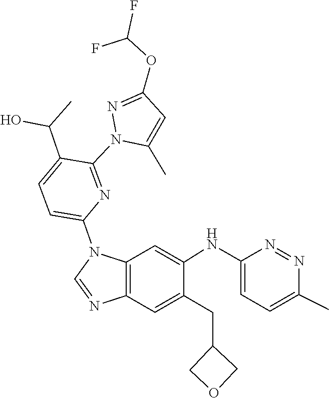 [SMILES: Cc1ccc(Nc2cc3c(cc2CC2COC2)ncn3-c2ccc(C(C)O)c(-n3nc(OC(F)F)cc3C)n2)nn1] | 1-[2-[3-(difluorometh- oxy)-5-methyl-pyr- azol-1-yl]-6-[6-[(6- methylpyridazin-3-yl)- amino]-5-(oxetan-3- ylmethyl)benzimid- azol-1-yl]-3-pyridyl]- ethanol | G.20 and H.10 | LC-MS: m/z = 563.4 [M + H]+, ESI pos ¹H NMR (400 MHz, METHANOL-d4) δ = 8.80 (s, 1H), 8.37-8.26 (m, 1H), 8.15-8.02 (m, 1H), 7.97-7.88 (m, 1H), 7.79-7.71 (m, 1H), 7.60-7.50 (m, 1H), 7.27 (d, J = 9.4 Hz, 1H), 6.93 (dt, J = 1.6, 73.3 Hz, 1H), 5.87 (d, J = 3.0 Hz, 1H), 4.90-4.80 (m, 1H), 4.03 (dd, J = 6.4, 12.7 Hz, 1H), 3.60- 3.43 (m, 3H), 2.93 (dd, J = 4.5, 15.0 Hz, 1H), 2.60 (dd, J = 9.8, 14.8 Hz, 1H), 2.53 (s, 3H), 2.24-2.13 (m, 1H), 2.09 (d, J = 2.6 Hz, 3H), 1.27 (d, J = 6.4 Hz, 3H) |
| 407 | 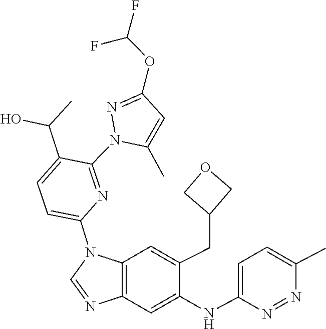 [SMILES: Cc1ccc(Nc2cc3ncn(-c4ccc(C(C)O)c(-n5nc(OC(F)F)cc5C)n4)c3cc2CC2COC2)nn1] | 1-[2-[3-(difluorometh- oxy)-5-methyl-pyr- azol-1-yl]-6-[5-[(6- methylpyridazin-3-yl)- amino]-6-(oxetan-3- ylmethyl)benzimid- azol-1-yl]-3-pyridyl]- ethanol | G.20 and H.10 | LC-MS: m/z = 563.4 [M + H]⁺, ESI pos ¹H NMR (400 MHz, METHANOL-d4) δ = 8.85 (s, 1H), 8.47 (d, J = 8.4 Hz, 1H), 8.09- 8.08 (m, 1H), 8.11 (s, 1H), 8.08-8.02 (m, 1H), 7.58-7.53 (m, 2H), 7.35 (d, J = 9.3 Hz, 1H), 7.09 (t, J = 73.3 Hz, 1H), 6.10 (s, 1H), 5.03-4.99 (m, 1H), 4.22 (dd, J = 6.2, 12.3 Hz, 1H), 3.68- 3.57 (m, 3H), 3.03 (dd, J = 4.8, 15.0 Hz, 1H), 2.71 (dd, J = 9.8, 14.9 Hz, 1H), 2.58 (s, 3H), 2.42 (s, 3H), 2.29 |
| | | | | (br d, J = 4.5 Hz, 1H), |
| | | | | 1.44 (dd, J = 1.4, 6.4 |
| | | | | Hz, 3H |

### Enzymatic Activity Assay

Assay description SIK1-3, RapidFire:

In the presence of SIK2 (resp. SIK1 or SIK3) and ATP the CHK-peptide (KKKVSRSGLYRSPSMPENLNRPR with C-terminal arginine amide modification) were phosphorylated at one of the four feasible serine's. Only one phosphorylation is observed under the assay conditions. 60 nl of each compound dilution series (12 point; dilution factor 3, generally 30 μM to 170 pM) in DMSO were transferred by acoustic dispensing to the assay plate and 30 min pre-incubated (ambient temperature) after the addition of 5 μl SIK1 (5 nM) resp. 5 μl SIK2 (0.5 nM) or 7 μl SIK3 (1.5 nM) in assay-buffer (12.5 mM HEPES (pH 7.0), 10 mM magnesium acetate, 0.005% BSA). 10 μM CHK-peptide solution and 5 μl 100 μM ATP for SIK1 & SIK2 resp. 3 μl for SIK3 in assay-buffer were added and incubated ambient for 45 min. 40 μl 0.125% formic acid in water were added to quench the reaction. RapidFire (RF) Mass Spectrometry was utilized for data generation as described below. The multiple charged species (3-5 charges) for the phosphorylated and non-phosphorylated form measured by MRM (Multiple Reaction Monitoring; API5000 or 6500+) or EIC (Extracted Ion Current; QToF) were summed up and the ratio calculated (sum phosphorylated species/sum all species) for data evaluation. Normalization was performed by Genedata software based on the non-inhibition control DMSO and the commercially available SIK inhibitor @ 1 μM YKL-05-099 (CAS number 1936529-65-5). The results of the assay are expressed in half-maximal inhibitory concentrations (IC50s) and are summarized below in Table 1.

### RapidFire Setup:

Samples were aspirated by vacuum for max. 600 ms and loaded to C4-cartridge (Agilent; #G9203A) for 3000 ms@1.5 ml/min with 0.1% formic acid in water. Afterwards samples were transferred to the APT5000 (APT6500+) or QToF mass spectrometer for 4000 ms@1.25 ml/min with 90% acetonitrile; 10% water; 0.007% TFA; 0.093 formic acid. The cartridge was reconditioned for additional 500 ms with 0.1% formic acid in water

### MS-Setup Sciex API5000/API6500+:

All MS analyses using the following MS-setup in MRM mode: Electrospray positive; Ion Spray Voltage: 4000V; Temperature: 550° C.; Collision Gas: 5; Curtain Gas: 15; Gas1: 40; Gas 2: 42; EP: 10

| Name | Q1 | Q3 | Time(ms) | DP | CE | CXP |
|---|---|---|---|---|---|---|
| CHK(4+) | 676.0 | 84.2 | 50 | 46 | 99 | 18 |
| CHK(5+) | 541.0 | 84.2 | 50 | 51 | 71 | 36 |
| pCHK(4+) | 696.0 | 84.2 | 50 | 66 | 105 | 18 |
| pCHK(5+) | 557.0 | 84.2 | 50 | 51 | 53 | 4 |

### MS-Setup Agilent QToF 6545

All MS analyses using the following MS-setup in Mode MS: Dual AJS Electrospray positive; VCap: 3000V; Drying & Sheath gas: 340° C.@81/min; Nebulizer: 60 psig; Nozzle Voltage: 2000V; Fragmentor: 130V; Skimmer: 35V; Oct1 RF Vpp: 700V; Ref masses on@5spectra/s

| Name | EIC | Width |
|---|---|---|
| CHK(3+) | 900.8345 | 50 ppm |
| CHK(4+) | 675.8789 | 50 ppm |
| CHK(5+) | 540.9048 | 50 ppm |
| pCHK(3+) | 927.4961 | 50 ppm |
| pCHK(4+) | 695.8747 | 50 ppm |

TABLE 1

| IC50 values for inhibition of SIK1, SIK2, and SIK3: | | | |
|---|---|---|---|
| | SIK1 RF | SIK2 RF | SIK3 RF |
| Example | IC50 (uM) | IC50 (uM) | IC50 (uM) |
| 1 | 0.152 | 0.1054 | 0.3584 |
| 15 | 0.018 | 0.0128 | 0.0554 |
| 16 | 0.692 | 0.3133 | 2.1808 |
| 17 | 0.327 | 0.1546 | 1.1374 |
| 18 | 1.001 | 0.9881 | 2.8782 |
| 19 | 0.068 | 0.0265 | 0.1164 |
| 20 | 0.012 | 0.0050 | 0.0198 |
| 21 | 0.192 | 0.1350 | 0.5427 |
| 22 | 0.970 | 0.8490 | 4.4815 |
| 23 | 3.643 | 3.8778 | 9.0118 |
| 24 | 0.353 | 0.1915 | 0.7266 |
| 25 | 0.777 | 0.6051 | 0.9868 |
| 26 | 0.806 | 0.6986 | 1.4396 |
| 27 | 0.535 | 0.4729 | 0.7606 |
| 28 | 0.326 | 0.2681 | 0.7134 |
| 32 | 0.373 | 0.2594 | 1.3961 |
| 33 | 0.320 | 0.4017 | 1.9412 |
| 34 | 0.508 | 0.3771 | 1.6825 |
| 35 | 13.442 | 6.0804 | 7.4714 |
| 36 | 23.938 | 9.6846 | 13.5555 |
| 37 | 0.015 | 0.0049 | 0.0224 |
| 38 | 0.158 | 0.0584 | 0.2590 |
| 39 | 0.152 | 0.0249 | 0.1776 |
| 40 | 1.032 | 0.5166 | 1.6602 |
| 41 | 0.197 | 0.0917 | 0.2999 |
| 42 | 1.303 | 1.0425 | 2.0577 |
| 53 | 2.155 | 0.3201 | 0.4049 |
| 55 | 1.288 | 0.1030 | 0.1301 |
| 62 | 1.666 | 0.1894 | 0.1759 |
| 63 | 0.093 | 0.0346 | 0.1515 |
| 64 | 0.059 | 0.0005 | 0.0006 |
| 65 | 0.652 | 0.0637 | 0.0819 |
| 67 | 0.767 | 0.1321 | 0.0979 |
| 68 | 0.179 | 0.0277 | 0.0297 |
| 71 | 0.005 | 0.0014 | 0.0022 |
| 72 | 0.005 | 0.0010 | 0.0021 |
| 74 | 0.045 | 0.0366 | 0.1901 |
| 75 | 1.443 | 0.0384 | 0.0063 |
| 76 | 7.391 | 0.3269 | 0.0662 |
| 77 | 0.028 | 0.0101 | 0.0157 |
| 78 | 0.033 | 0.0114 | 0.0204 |
| 81 | 0.009 | 0.0023 | 0.0008 |
| 82 | 0.005 | 0.0022 | 0.0024 |
| 83 | 0.002 | 0.0015 | 0.0018 |
| 84 | 0.007 | 0.0053 | 0.0021 |
| 85 | 1.070 | 0.8181 | 1.9111 |
| 86 | 0.062 | 0.0060 | 0.0015 |
| 87 | 0.022 | 0.0096 | 0.0029 |
| 88 | 0.062 | 0.0022 | 0.0008 |
| 89 | 0.095 | 0.0012 | 0.0007 |
| 90 | 0.053 | 0.0009 | 0.0005 |
| 93 | 0.097 | 0.0005 | 0.0006 |
| 94 | 0.024 | 0.0166 | 0.0549 |
| 97 | 0.007 | 0.0002 | 0.0005 |
| 98 | 0.005 | 0.0042 | 0.0134 |
| 103 | 0.035 | 0.0288 | 0.0175 |
| 104 | 0.616 | 0.0104 | 0.0026 |
| 105 | 1.338 | 0.7826 | 0.2362 |
| 106 | 0.869 | 0.0211 | 0.0025 |
| 107 | 2.614 | 1.3681 | 3.5005 |
| 108 | 3.823 | 0.0426 | 0.0064 |
| 109 | 1.798 | 0.0278 | 0.0186 |
| 110 | 0.026 | 0.0002 | 0.0002 |
| 111 | 0.011 | 0.0014 | 0.0008 |
| 113 | 0.319 | 0.0760 | 0.0341 |
| 114 | 0.049 | 0.0470 | 0.6060 |
| 115 | 0.283 | 0.0039 | 0.0007 |
| 116 | 1.615 | 0.0471 | 0.0096 |
| 117 | 3.533 | 1.0245 | 0.3646 |
| 118 | 2.774 | 0.0364 | 0.0071 |
| 119 | 0.012 | 0.0018 | 0.0016 |
| 120 | 2.212 | 1.3104 | 0.3072 |
| 121 | 1.200 | 0.0224 | 0.0036 |
| 122 | 3.465 | 0.0330 | 0.0069 |
| 123 | 0.013 | 0.0099 | 0.0059 |
| 124 | 1.520 | 0.0360 | 0.0101 |
| 125 | 0.981 | 0.8630 | 0.8952 |
| 126 | 0.065 | 0.0745 | 0.2278 |
| 127 | 0.457 | 0.0137 | 0.0155 |
| 128 | 0.193 | 0.0895 | 0.1548 |
| 129 | 0.472 | 0.0039 | 0.0012 |
| 130 | 0.589 | 0.2145 | 0.3395 |
| 133 | 0.447 | 0.0077 | 0.0022 |
| 134 | 0.426 | 0.3447 | 1.2932 |
| 135 | 27.667 | 0.5889 | 0.1672 |
| 136 | 0.345 | 0.0130 | 0.0039 |
| 137 | 5.410 | 1.6396 | 0.7460 |
| 138 | 3.061 | 2.4523 | 2.7014 |
| 139 | 3.021 | 0.0504 | 0.0096 |
| 140 | 0.051 | 0.0388 | 0.1741 |
| 143 | 0.103 | 0.0007 | 0.0013 |
| 144 | 8.396 | 5.9159 | 9.2367 |
| 145 | 0.552 | 0.0090 | 0.0014 |
| 146 | 0.227 | 0.0346 | 0.0078 |
| 148 | 0.663 | 0.0103 | 0.0012 |
| 149 | 1.291 | 0.0084 | 0.0019 |
| 150 | 24.370 | 0.2070 | 0.0335 |
| 151 | 13.566 | 4.7077 | 1.8844 |
| 152 | 5.044 | 0.0821 | 0.0161 |
| 155 | 0.361 | 0.0902 | 0.0265 |
| 158 | 2.152 | 0.0419 | 0.0128 |
| 159 | 23.848 | 0.3059 | 0.0702 |
| 160 | 0.223 | 0.0032 | 0.0012 |
| 161 | 0.450 | 0.1807 | 0.0661 |
| 162 | 8.569 | 0.1981 | 0.0469 |
| 163 | >30.000 | 0.9640 | 0.1805 |
| 164 | >30.000 | 3.3027 | 0.9626 |
| 165 | 13.316 | 0.3587 | 0.0955 |
| 166 | 0.681 | 0.0832 | 0.0408 |
| 167 | 0.040 | 0.0009 | 0.0012 |
| 168 | 1.760 | 0.0399 | 0.0086 |
| 169 | 0.613 | 0.1354 | 0.0852 |
| 170 | 0.216 | 0.0021 | 0.0010 |
| 171 | 0.009 | 0.0003 | 0.0007 |
| 172 | 2.616 | 2.0923 | 1.8508 |
| 173 | 3.486 | 0.1980 | 0.0539 |
| 174 | 12.079 | 0.1512 | 0.0290 |
| 175 | 0.054 | 0.0575 | 0.2029 |
| 176 | 0.035 | 0.0006 | 0.0013 |
| 177 | 0.253 | 0.0028 | 0.0031 |
| 178 | 27.009 | 6.3400 | 10.0000 |
| 179 | >30.000 | 0.4136 | 0.0743 |
| 182 | 15.364 | 0.0889 | 0.0257 |
| 183 | 2.834 | 0.0945 | 0.0175 |
| 184 | 1.986 | 2.4963 | 1.8968 |
| 185 | 0.006 | 0.0003 | 0.0007 |
| 186 | −n.d. | 1.2390 | 1.0613 |
| 187 | 0.070 | 0.0020 | 0.0056 |
| 188 | 0.087 | 0.0700 | 0.2131 |
| 189 | 0.186 | 0.0039 | 0.0047 |
| 190 | 0.084 | 0.0508 | 0.3816 |
| 191 | 0.053 | 0.0027 | 0.0022 |
| 192 | 0.196 | 0.0031 | 0.0040 |
| 193 | 0.068 | 0.0032 | 0.0019 |
| 194 | 3.333 | 0.2884 | 0.0295 |
| 195 | 0.020 | 0.0031 | 0.0014 |
| 197 | 0.015 | 0.0023 | 0.0009 |
| 198 | 0.391 | 0.0070 | 0.0018 |
| 199 | 0.245 | 0.1708 | 0.3709 |
| 201 | 0.111 | 0.0621 | 0.3215 |
| 202 | 1.016 | 0.5002 | 0.2048 |
| 203 | 14.434 | 0.5196 | 0.0554 |
| 204 | 9.948 | 0.1412 | 0.0344 |
| 205 | 0.003 | 0.0003 | 0.0003 |
| 206 | 0.072 | 0.0045 | 0.0053 |
| 207 | 0.027 | 0.0037 | 0.0026 |
| 208 | 0.462 | 0.0297 | 0.0048 |
| 212 | 0.453 | 0.2920 | 1.5788 |
| 213 | 7.585 | 1.5220 | 0.1736 |
| 214 | 1.429 | 0.0207 | 0.0035 |
| 215 | 0.347 | 0.3707 | 1.8646 |
| 216 | 10.840 | 2.2421 | 0.4757 |
| 219 | 2.205 | 0.0068 | 0.0042 |
| 220 | 6.742 | 0.1537 | 0.0462 |
| 223 | >30.000 | 1.5695 | 0.3349 |
| 226 | >30.000 | 0.0621 | 0.0217 |
| 227 | 4.175 | 0.2248 | 0.0286 |
| 228 | 0.091 | 0.0010 | 0.0009 |
| 229 | 0.223 | 0.0015 | 0.0004 |
| 230 | 7.093 | 0.0724 | 0.0197 |
| 231 | 1.047 | 0.0129 | 0.0020 |
| 234 | 0.756 | 0.0093 | 0.0011 |
| 235 | 0.647 | 0.2448 | 0.2012 |
| 236 | 0.084 | 0.0003 | 0.0003 |
| 237 | 7.821 | 0.0497 | 0.0528 |
| 239 | >30.000 | 1.3860 | 0.3286 |
| 240 | 0.043 | 0.0003 | 0.0002 |
| 241 | 0.173 | 0.0018 | 0.0009 |
| 242 | 0.843 | 0.0095 | 0.0018 |
| 245 | 6.408 | 0.0725 | 0.0674 |
| 246 | 7.777 | 0.0295 | 0.0054 |
| 247 | 1.445 | 0.1736 | 0.0461 |
| 248 | 0.242 | 0.0018 | 0.0007 |
| 249 | 0.054 | 0.0022 | 0.0008 |
| 250 | 0.985 | 0.0252 | 0.0048 |
| 251 | 0.063 | 0.0003 | 0.0004 |
| 252 | 5.616 | 0.0468 | 0.0804 |
| 253 | 8.973 | 0.1611 | 0.0345 |
| 254 | 5.108 | 0.1504 | 0.0373 |
| 255 | 12.808 | 0.1936 | 0.0304 |
| 256 | 1.530 | 0.0220 | 0.0048 |
| 263 | 0.330 | 0.1438 | 0.1582 |
| 264 | 0.851 | 0.0026 | 0.0015 |
| 265 | 0.270 | 0.0016 | 0.0017 |
| 266 | 0.136 | 0.0010 | 0.0014 |
| 269 | 0.151 | 0.2107 | 1.0341 |
| 270 | 0.134 | 0.0008 | 0.0002 |
| 271 | 0.895 | 0.0073 | 0.0021 |
| 272 | 0.193 | 0.0024 | 0.0012 |
| 273 | 0.115 | 0.0012 | 0.0015 |
| 274 | 0.120 | 0.0012 | 0.0011 |
| 275 | 0.800 | 0.0061 | 0.0159 |
| 276 | 0.082 | 0.0023 | 0.0018 |
| 277 | 0.521 | 0.0020 | 0.0018 |
| 278 | 0.249 | 0.0020 | 0.0047 |
| 279 | 0.532 | 0.0047 | 0.0029 |
| 280 | 0.039 | 0.0263 | 0.1491 |
| 281 | 0.046 | 0.0006 | 0.0012 |
| 282 | 0.007 | 0.0004 | 0.0008 |
| 283 | 1.055 | 0.0215 | 0.0079 |
| 284 | 0.167 | 0.0021 | 0.0019 |
| 285 | 0.497 | 0.0116 | 0.0064 |
| 287 | 0.109 | 0.0008 | 0.0004 |
| 288 | 1.597 | 0.0132 | 0.0058 |
| 289 | 0.595 | 0.0124 | 0.0058 |
| 290 | 0.054 | 0.0006 | 0.0005 |
| 291 | 0.001 | 0.0012 | 0.0119 |
| 292 | 0.617 | 0.0342 | 0.0242 |
| 293 | 0.132 | 0.0060 | 0.0028 |
| 294 | 0.002 | 0.0003 | 0.0030 |
| 295 | 0.010 | 0.0196 | 0.2348 |
| 296 | 0.137 | 0.0770 | 0.0900 |
| 297 | 0.010 | 0.0062 | 0.0148 |
| 298 | 1.509 | 0.0274 | 0.0030 |
| 299 | 0.273 | 0.0024 | 0.0007 |
| 301 | 0.664 | 0.0035 | 0.0020 |
| 302 | 0.409 | 0.0007 | 0.0011 |
| 303 | 0.030 | 0.0004 | 0.0004 |
| 304 | 0.273 | 0.0954 | 0.0372 |
| 305 | 0.155 | 0.0017 | 0.0021 |
| 306 | 0.208 | 0.0016 | 0.0007 |
| 307 | 0.679 | 0.0078 | 0.0028 |
| 308 | 0.058 | 0.0004 | 0.0004 |
| 309 | 0.027 | 0.0003 | 0.0004 |
| 310 | 0.093 | 0.0011 | 0.0005 |
| 311 | 0.017 | 0.0003 | 0.0003 |
| 312 | 0.250 | 0.0796 | 0.0856 |
| 313 | 0.138 | 0.0011 | 0.0014 |
| 314 | 0.070 | 0.0524 | 0.0651 |
| 315 | 1.086 | 0.0073 | 0.0029 |
| 316 | 0.975 | 0.0151 | 0.0046 |
| 317 | 0.961 | 0.0076 | 0.0026 |
| 318 | 0.327 | 0.0501 | 0.0650 |
| 319 | 2.118 | 0.0067 | 0.0070 |
| 320 | 4.865 | 0.0653 | 0.0095 |
| 321 | 1.095 | 0.0192 | 0.0035 |
| 322 | 0.966 | 0.0163 | 0.0024 |
| 323 | 2.534 | 0.0485 | 0.0071 |
| 324 | 0.577 | 0.0105 | 0.0008 |
| 325 | 1.122 | 0.0214 | 0.0034 |
| 326 | 1.675 | 0.0671 | 0.0078 |
| 327 | 0.040 | 0.0072 | 0.0041 |
| 328 | 0.127 | 0.0661 | 0.0365 |
| 329 | 1.737 | 0.0590 | 0.0100 |
| 330 | 0.113 | 0.0260 | 0.0513 |
| 331 | 3.344 | 0.0715 | 0.0093 |
| 332 | 0.316 | 0.0053 | 0.0028 |
| 333 | 0.041 | 0.0251 | 0.0194 |
| 334 | 0.003 | 0.0016 | 0.0034 |
| 335 | 0.010 | 0.0010 | 0.0003 |
| 336 | 0.029 | 0.0286 | 0.1389 |
| 337 | 1.200 | 0.0083 | 0.0048 |
| 338 | 0.087 | 0.0272 | 0.0276 |
| 339 | 0.032 | 0.0297 | 0.3008 |
| 340 | 0.042 | 0.0203 | 0.0179 |
| 341 | 0.014 | 0.0151 | 0.1240 |
| 342 | 0.022 | 0.0095 | 0.0161 |
| 343 | 0.005 | 0.0220 | 0.0630 |
| 344 | 1.569 | 0.0202 | 0.0032 |
| 345 | 17.199 | 0.0489 | 0.0602 |
| 346 | 0.133 | 0.0010 | 0.0014 |
| 347 | 1.122 | 0.0063 | 0.0039 |
| 348 | 14.523 | 0.0322 | 0.0371 |
| 349 | 0.432 | 0.0053 | 0.0066 |
| 350 | 3.801 | 0.0670 | 0.0606 |
| 351 | 3.393 | 0.0354 | 0.0093 |
| 352 | 2.335 | 0.0165 | 0.0241 |
| 353 | 0.230 | 0.0606 | 0.2176 |
| 354 | 0.225 | 0.0786 | 0.0759 |
| 355 | 1.049 | 0.0053 | 0.0036 |
| 356 | 0.245 | 0.0009 | 0.0017 |
| 357 | 0.019 | 0.0010 | 0.0017 |
| 358 | 0.051 | 0.0025 | 0.0084 |
| 359 | 1.166 | 0.0253 | 0.0046 |
| 360 | 2.315 | 0.0423 | 0.0079 |
| 361 | 0.321 | 0.0639 | 0.0511 |
| 362 | 0.012 | 0.0087 | 0.0267 |
| 363 | 0.152 | 0.0012 | 0.0010 |
| 364 | 0.008 | 0.0029 | 0.0117 |
| 365 | 0.020 | 0.0446 | 0.4455 |
| 366 | 0.133 | 0.0582 | 0.0233 |
| 367 | 0.088 | 0.0015 | 0.0008 |
| 368 | 0.106 | 0.0771 | 1.1008 |
| 369 | 1.322 | 0.0082 | 0.0041 |
| 370 | 0.496 | 0.0071 | 0.0020 |
| 371 | 0.145 | 0.0847 | 0.4330 |
| 372 | 1.221 | 0.0085 | 0.0036 |
| 373 | 0.184 | 0.0008 | 0.0007 |
| 374 | 0.088 | 0.0003 | 0.0004 |
| 375 | 0.998 | 0.0060 | 0.0087 |
| 376 | 0.014 | 0.0096 | 0.0420 |
| 377 | 0.047 | 0.0315 | 0.0432 |
| 378 | 0.079 | 0.0325 | 0.0217 |
| 379 | 0.001 | 0.0002 | 0.0004 |
| 380 | 0.081 | 0.0166 | 0.0124 |
| 381 | 0.055 | 0.0327 | 0.1955 |
| 382 | 0.207 | 0.0005 | 0.0006 |
| 383 | 0.005 | 0.0002 | 0.0005 |
| 384 | 0.215 | 0.0413 | 0.0802 |
| 385 | 0.022 | 0.0028 | 0.0025 |
| 386 | 0.069 | 0.0012 | 0.0012 |
| 387 | 0.462 | 0.0023 | 0.0018 |
| 388 | 0.011 | 0.0004 | 0.0007 |
| 389 | 0.042 | 0.0079 | 0.0051 |
| 390 | 5.888 | 0.0272 | 0.0160 |
| 391 | 0.023 | 0.0045 | 0.0020 |
| 392 | 0.010 | 0.0020 | 0.0045 |
| 393 | 0.750 | 0.0252 | 0.0055 |
| 394 | 0.013 | 0.0003 | 0.0006 |
| 395 | 0.004 | 0.0029 | 0.0397 |
| 396 | 1.118 | 0.0135 | 0.0056 |
| 397 | 0.037 | 0.0205 | 0.1719 |
| 398 | 0.271 | 0.0039 | 0.0020 |
| 399 | 0.039 | 0.0006 | 0.0010 |
| 400 | 0.002 | 0.0006 | 0.0078 |
| 401 | 1.363 | 0.0104 | 0.0042 |
| 402 | 0.005 | 0.0002 | 0.0003 |
| 403 | 0.458 | 0.0247 | 0.0322 |
| 404 | 0.041 | 0.0005 | 0.0005 |
| 405 | 0.039 | 0.0198 | 0.1501 |
| 406 | 0.047 | 0.0134 | 0.1444 |
| 407 | 0.321 | 0.0129 | 0.0229 |

### Specific Numbered Embodiments

1. A compound of formula (I)

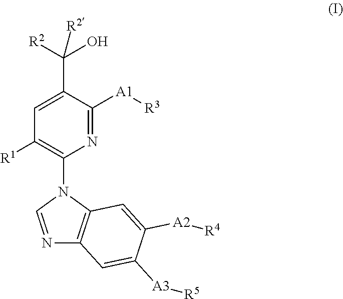

wherein
R¹ is hydrogen or halogen;
R² and R^{2′} are independently selected from hydrogen, alkyl, cyclopropyl, haloalkyl and alkoxyalkyl;
A1 is —O—, —NR⁶—, —(C═O)— or a bond;
R⁶ is hydrogen or alkyl;
R³ is hydroxyalkyl, heterocycloalkyl, heteroaryl, phenyl or cycloalkylalkyl, wherein heterocycloalkyl, heteroaryl, phenyl and cycloalkylalkyl are optionally substituted with 1, 2 or 3 substituents independently selected from R⁷; if A1 is —(C═O)—, then R³ can also be haloalkylamino;
each R⁷ is independently selected from alkoxy, alkylamino, alkyl, aminocarbonyl, amino, cyano, cycloalkylamino, haloalkyl, halocycloalkyl, halogen, heteroaryl, hydroxycarbonylamino, alkoxyalkyl, alkylaminocarbonyl, alkylsulfonyl, alkoxycarbonimidoyl, aminocarbonyl, hydroxy, cycloalkylalkyl, haloalkoxy, heterocycloalkyl and cycloalkyl;
A2 is —O—, —NH— or a bond;
R⁴ is hydrogen, alkyl, haloalkyl, alkoxyalkyl, dialkylaminoalkyl, cycloalkyl, cycloalkylcarbonyl, aryl, heteroaryl, heteroarylalkyl, heterocycloalkyl or heterocycloalkylalkyl; wherein aryl, cycloalkyl, heteroaryl, heteroarylalkyl, heterocycloalkyl and heterocycloalkylalkyl are optionally substituted with 1, 2 or 3 substituents independently selected from R⁸; if A2 is a bond, then R⁴ can also be halogen or cyano;
each R¹ is independently selected from alkyl, halogen, cyano, alkylsulfonyl, alkylaminocarbonyl, heterocycloalkyl and alkoxyheterocycloalkylalkyl;
A3 is —O—, —NR¹⁰— or a bond;
R⁵ is hydrogen, alkyl, alkylsulfonyl, cycloalkylcarbonyl, heterocycloalkylalkyl, heterocycloalkylalkylcarbonyl, aryl, heteroaryl or heterocycloalkyl, wherein cycloalkylcarbonyl, aryl, heteroaryl and heterocycloalkyl are optionally substituted with 1, 2 or 3 substituents independently selected from R⁹; if A3 is a bond, then R¹ can also be halogen or cyano;
each R⁹ is independently selected from alkoxy, halogen, dialkylaminocarbonyl, alkyl, alkoxyalkoxy, alkoxyheterocycloalkylalkyl, alkoxyheterocycloalkylcarbonyl, haloalkyl, haloalkoxy, heterocycloalkylalkoxy, heterocycloalkyl, heterocycloalkyloxy, hydroxy, hydroxyalkyl, alkylheterocycloalkyl, (haloalkyl)cycloalkyl, alkylheterocycloalkylalkyl, heterocycloalkylalkyl, alkylsulfonyl, (alkyl)heterocycloalkyl, alkylheterocycloalkyloxy, heterocycloalkylheterocycloalkyl, heterocycloalkylheterocycloalkyl, CH₃—O—(CH₂—CH₂—O)ₙ—, alkylaminocarbonyl and cyano; wherein n is selected from 5, 6, 7, 8 and 9; and
R¹⁰ is hydrogen or alkylcarbonyl;
or a pharmaceutically acceptable salt thereof.

2. A compound of formula (I)

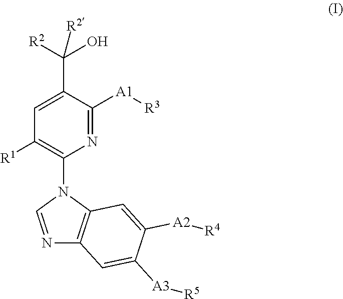

wherein
R¹ is hydrogen or halogen;
R² and R^{2′} are independently selected from hydrogen, alkyl, cyclopropyl, haloalkyl and alkoxyalkyl;
A1 is —O—, —NR⁶—, —(C═O)— or a bond;
R⁶ is hydrogen or alkyl;
R³ is alkyl, hydroxyalkyl, heterocycloalkyl, heteroaryl, phenyl or cycloalkylalkyl, wherein heterocycloalkyl, heteroaryl, phenyl and cycloalkylalkyl are optionally substituted with 1, 2 or 3 substituents independently selected from R⁷; if A1 is —(C═O)—, then R³ can also be haloalkylamino; each R⁷ is independently selected from alkoxy, alkylamino, alkyl, aminocarbonyl, amino, cyano, cycloalkylamino, haloalkyl, halocycloalkyl, halogen, heteroaryl, hydroxycarbonylamino, alkoxyalkyl, alkylaminocarbonyl, alkylsulfonyl, alkoxycarbonimidoyl, aminocarbonyl, hydroxy, cycloalkylalkyl, haloalkoxy, heterocycloalkyl and cycloalkyl;
A2 is —O—, —NH— or a bond;
R⁴ is hydrogen, alkyl, haloalkyl, alkoxyalkyl, dialkylaminoalkyl, cycloalkylcarbonyl, heteroaryl, heteroarylalkyl, heterocycloalkyl or heterocycloalkylalkyl; wherein heteroaryl, heteroarylalkyl, heterocycloalkyl and heterocycloalkylalkyl are optionally substituted with 1, 2 or 3 substituents independently selected from R⁸; if A2 is a bond, then R⁴ can also be halogen or cyano;
each R⁸ is independently selected from alkyl, halogen, cyano, alkylsulfonyl, alkylaminocarbonyl, heterocycloalkyl and alkoxyheterocycloalkylalkyl;
A3 is —O—, —NH— or a bond;
R⁵ is hydrogen, alkyl, alkylsulfonyl, cycloalkylcarbonyl, heterocycloalkylalkyl, heterocycloalkylalkylcarbonyl, heteroaryl or heterocycloalkyl, wherein heteroaryl and heterocycloalkyl are optionally substituted with 1, 2 or 3 substituents independently selected from R⁹; if A3 is a bond, then R⁵ can also be halogen or cyano;
each R⁹ is independently selected from alkoxy, halogen, dialkylaminocarbonyl, alkyl, alkoxyalkoxy, alkoxyheterocycloalkylalkyl, alkoxyheterocycloalkylcarbonyl, haloalkyl, haloalkoxy, heterocycloalkylalkoxy, heterocycloalkyl, heterocycloalkyloxy, hydroxy, alkylheterocycloalkyl, alkylheterocycloalkylalkyl, heterocycloalkylalkyl, alkylsulfonyl, (alkyl)heterocycloalkyl, alkylheterocycloalkyloxy, heterocycloalkylheterocycloalkyl, heterocycloalkylheterocycloalkyl, CH₃—O—(CH₂—CH₂—O)ₙ—, alkylaminocarbonyl and cyano; wherein n is selected from 5, 6, 7, 8 and 9; or a pharmaceutically acceptable salt thereof.

3. A compound according to embodiment 1 or 2, wherein R¹ is hydrogen or chloro.

4. A compound according to embodiment 1 to 3, wherein R¹ is hydrogen.

5. A compound according to any one of embodiments 1 to 4, wherein R¹ is halogen, in particular chloro.

6. A compound according to any one of embodiments 1 to 5, wherein R² and R^{2′} are independently selected from hydrogen, methyl, ethyl, cyclopropyl, difluoromethyl, trifluoromethyl and methoxymethyl.

7. A compound according to any one of embodiments 1 to 6, wherein R² and R^{2′} are independently selected from hydrogen, methyl, difluoromethyl, trifluoromethyl and methoxymethyl.

8. A compound according to any one of embodiments 1 to 7, wherein R² is hydrogen and R^{2′} is independently selected from hydrogen, methyl, ethyl, cyclopropyl, difluoromethyl, trifluoromethyl and methoxymethyl.

9. A compound according to any one of embodiments 1 to 8, wherein R² is alkyl and R^{2′} is hydrogen.

10. A compound according to any one of embodiments 1 to 9, wherein R² is methyl and R^{2′} is hydrogen.

11. A compound according to any one of embodiments 1 to 10, wherein A1 is —O—, —NR⁶— or a bond.

12. A compound according to any one of embodiments 1 to 10, wherein A1 is —O— or a bond.

13. A compound according to any one of embodiments 1 to 10, wherein A1 is —O—.

14. A compound according to any one of embodiments 1 to 10, wherein A1 is a bond.

15. A compound according to any one of embodiments 1 to 10, wherein A1 is —NR⁶—.

16. A compound according to any one of embodiments 1 to 10, wherein A1 is —(C═O)—.

17. A compound according to any one of embodiments 1 to 16, wherein R⁶ is hydrogen or methyl.

18. A compound according to any one of embodiments 1 to 17, wherein R⁶ is hydrogen.

19. A compound according to any one of embodiments 1 to 18, wherein R³ is methyl, ethyl, butyl, pentyl, hydroxypentyl, hydroxybutyl, phenyl, cyclopropylmethyl, heterocycloalkyl or heteroaryl, and wherein phenyl, heterocycloalkyl and heteroaryl are optionally substituted with 1, 2 or 3 substituents independently selected from R⁷; if A1 is —(C═O)—, then R³ can also be trifluoroethylamino;
each R⁷ is independently selected from aminocarbonyl, amino, cyano, methoxy, ethoxy, chloro, fluoro, hydroxy, trifluoromethyl, trifluoroethyl, difluoromethyl, methylsulfonyl, methylaminocarbonyl, methoxyethyl, cyclopropylamino, difluoromethoxy, oxetan-3-yl, methylamino, 1H-pyrazol-4-yl, difluorocyclopropyl, methyl, ethyl, cyclopropylmethyl and cyclobutylmethyl.

20. A compound according to any one of embodiments 1 to 18, wherein R³ is hydroxypentyl, hydroxybutyl, phenyl, cyclopropylmethyl, heterocycloalkyl or heteroaryl, and wherein phenyl, heterocycloalkyl and heteroaryl are optionally substituted with 1, 2 or 3 substituents independently selected from R⁷; if A1 is —(C═O)—, then R³ can also be trifluoroethylamino;
each R⁷ is independently selected from aminocarbonyl, amino, cyano, methoxy, ethoxy, chloro, fluoro, hydroxy, trifluoromethyl, trifluoroethyl, difluoromethyl, methylsulfonyl, methylaminocarbonyl, methoxyethyl, cyclopropylamino, difluoromethoxy, oxetan-3-yl, methylamino, 1H-pyrazol-4-yl, difluorocyclopropyl, methyl, ethyl, cyclopropylmethyl and cyclobutylmethyl.

21. A compound according to any one of embodiments 1 to 20, wherein R³ is methyl, ethyl, butyl, pentyl, hydroxypentyl, hydroxybutyl, phenyl, cyclopropylmethyl, heterocycloalkyl or heteroaryl, and wherein phenyl, heterocycloalkyl and heteroaryl are optionally substituted with 1, 2 or 3 substituents independently selected from R7; and
wherein each R⁷ is independently selected from aminocarbonyl, amino, cyano, methoxy, ethoxy, chloro, fluoro, hydroxy, trifluoromethyl, trifluoroethyl, difluoromethyl, methylsulfonyl, methylaminocarbonyl, methoxyethyl, cyclopropylamino, difluoromethoxy, oxetan-3-yl, methylamino, 1H-pyrazol-4-yl, difluorocyclopropyl, methyl, ethyl, cyclopropylmethyl and cyclobutylmethyl.

22. A compound according to any one of embodiments 1 to 21, wherein R³ is haloalkylamino.

23. A compound according to any one of embodiments 1 to 22, wherein the heterocycloalkyl of substituent R³ is selected from morpholino, pyrrolidinyl, piperidyl, 2-oxopyrrolidinyl, (1,1-dioxo-1,2-thiazolidinyl), (4,5,6,7-tetrahydropyrazolo[4,3-c]pyridinyl), pyrrolidinyl, [rac-(3aR,6aS)-2,3,3a,5,6,6a-hexahydro-1H-pyrrolo[3,2-b]pyrrolyl], [rac-(3aS,6aR)-2,3,3a,5,6,6a-hexahydro-1H-pyrrolo[3,2-b]pyrrolyl], [3-oxo-piperazinyl], (4-oxo-6,7-dihydro-5H-pyrazolo[1,5-a]pyrazinyl), (6,7-dihydro-4H-pyrazolo[4,3-c]pyridinyl), azetidinyl, pyrrolidinyl, (3-oxo-1,5,6,8-tetrahydrooxazolo[3,4-a]pyrazinyl), piperazinyl, 4,7-diazaspiro[2.5]octanyl, (2-oxa-5,8-diazaspiro[3.5]nonanyl), 3-azabicyclo[3.2.0]heptanyl), (5-azaspiro[2.4]heptanyl), (2-azabicyclo[2.2.1]heptanyl), morpholinyl, 4-oxa-7-azaspiro[2.5]octanyl, (3-azabicyclo[3.1.0]hexanyl), (6,7-dihydro-4H-pyrazolo[4,3-c]pyridinyl), 5,6-dihydro-4H-pyrazolo[4,3-c]pyridin-1-yl, 6-azaspiro[3.4]octan-6-yl and 2-oxa-7-azaspiro[3.4]octanyl.

24. A compound according to any one of embodiments 1 to 23, wherein the heteroaryl of substituent R³ is selected from 2-oxo-pyridyl, 2-oxo-3-imidazol-1-yl, 1,2,4-triazol-3-yl, pyrazolyl, pyridyl, pyridazinyl, isoxazolyl, pyrimidinyl, 1H-benzotriazolyl, furyl, [6-oxo-1H-pyridazinyl] and triazolyl.

25. A compound according to any one of embodiments 1 to 24, wherein the heterocycloalkyl of substituent R³ is selected from morpholino, pyrrolidinyl, piperidyl, 2-oxopyrrolidinyl, (1,1-dioxo-1,2-thiazolidinyl), (4,5,6,7-tetrahydropyrazolo[4,3-c]pyridinyl), pyrrolidinyl, [rac-(3aR,6aS)-2,3,3a,5,6,6a-hexahydro-1H-pyrrolo[3,2-b]pyrrolyl], [rac-(3aS,6aR)-2,3,3a,5,6,6a-hexahydro-1H-pyrrolo[3,2-b]pyrrolyl], [3-oxo-piperazinyl], (4-oxo-6,7-dihydro-5H-pyrazolo[1,5-a]pyrazinyl), (6,7-dihydro-4H-pyrazolo[4,3-c]pyridinyl), azetidinyl, pyrrolidinyl, (3-oxo-1,5,6,8-tetrahydrooxazolo[3,4-a]pyrazinyl), piperazinyl, 4,7-diazaspiro[2.5]octanyl, (2-oxa-5,8-diazaspiro[3.5]nonanyl), 3-azabicyclo[3.2.0]heptanyl), (5-azaspiro[2.4]heptanyl), (2-azabicyclo[2.2.1]heptanyl), morpholinyl, 4-oxa-7-azaspiro[2.5]octanyl, (3-azabicyclo[3.1.0]hexanyl), (6,7-dihydro-4H-pyrazolo[4,3-c]pyridinyl) and 2-oxa-7-azaspiro[3.4]octanyl; and wherein the heteroaryl of substituent R³ is selected from 2-oxo-pyridyl, pyrazolyl, pyridyl, pyridazinyl, isoxazolyl, pyrimidinyl, 1H-benzotriazolyl, furyl, [6-oxo-1H-pyridazinyl] and triazolyl.

26. A compound according to any one of embodiments 1 to 25, wherein the heterocycloalkyl of substituent R³ is selected from morpholino, pyrrolidin-1-yl, 1-piperidyl, 2-oxopyrrolidin-1-yl, (1,1-dioxo-1,2-thiazolidin-2-yl), (4,5,6,7-tetrahydropyrazolo[4,3-c]pyridin-1-yl), [rac-(3aR,6aS)-2,3,3a,5,6,6a-hexahydro-1H-pyrrolo[3,2-b]pyrrol-4-yl], [rac-(3aS,6aR)-2,3,3a,5,6,6a-hexahydro-1H-pyrrolo[3,2-b]pyrrol-4-yl], [3-oxo-piperazin-1-yl], (4-oxo-6,7-dihydro-5H-pyrazolo[1,5-a]pyrazin-3-yl), (6,7-dihydro-4H-pyrazolo[4,3-c]pyridin-1-yl), azetidin-1-yl, pyrrolidin-3-yl, (3-oxo-1,5,6,8-tetrahydrooxazolo[3,4-a]pyrazin-7-yl), piperazin-1-yl, (4,7-diazaspiro[2.5]octan-7-yl), (2-oxa-5,8-diazaspiro[3.5]nonan-8-yl), 3-azabicyclo[3.2.0]heptan-3-yl), (5-azaspiro[2.4]heptan-5-yl), (2-azabicyclo[2.2.1]heptan-2-yl), morpholin-4-yl, 4-oxa-7-azaspiro[2.5]octan-7-yl, (3-azabicyclo[3.1.0]hexan-3-yl), (6,7-dihydro-4H-pyrazolo[4,3-c]pyridin-1-yl) and 2-oxa-7-azaspiro[3.4]octan-7-yl; and
wherein the heteroaryl substituent of R³ is selected from 2-oxo-4-pyridyl, pyrazol-1-yl, pyrazol-3-yl, 3-pyridyl, pyridazin-4-yl, pyrazol-4-yl, isoxazol-4-yl, 4-pyridyl, pyrimidin-5-yl, 1H-benzotriazol-4-yl, 3-furyl, [6-oxo-1H-pyridazin-5-yl], 2-furyl, triazol-2-yl, triazol-4-yl and triazol-1-yl.

27. A compound according to any one of embodiments 1 to 26, wherein R³ is cycloalkylalkyl, heterocycloalkyl or heteroaryl, wherein heterocycloalkyl, heteroaryl and cycloalkylalkyl are optionally substituted with 1, 2 or 3 substituents independently selected from R7; and
wherein each R⁷ is independently selected from alkyl, cyano, haloalkyl, alkoxy, alkylsulfonyl, alkoxycarbonimidoyl and haloalkyloxy.

28. A compound according to any one of embodiments 1 to 27, wherein R³ is cyclopropylmethyl, heterocycloalkyl or heteroaryl, wherein heterocycloalkyl, heteroaryl and cycloalkylalkyl are optionally substituted with 1, 2 or 3 substituents independently selected from R⁷; and
wherein each R⁷ is independently selected from methyl, cyano, trifluoromethyl, difluoromethyl, trifluoroethyl, ethoxy, methoxy, methylsulfonyl, methoxycarbonimidoyl, difluoroethoxy and oxetan-3-yl.

29. A compound according to any one of embodiments 1 to 28, wherein the heterocycloalkyl of substituent R³ is selected from (4,5,6,7-tetrahydropyrazolo[4,3-c]pyridin-1-yl), pyrrolidin-1-yl, [rac-(3aR,6aS)-2,3,3a,5,6,6a-hexahydro-1H-pyrrolo[3,2-b]pyrrol-4-yl], [3-oxo-piperazin-1-yl], and (6,7-dihydro-4H-pyrazolo[4,3-c]pyridin-1-yl); and
wherein the heteroaryl of substituent R³ is selected from pyrazol-1-yl, pyrazol-3-yl, pyrazol-4-yl, 3-pyridyl, 4-pyridyl, triazol-1-yl and triazol-4-yl.

30. A compound according to any one of embodiments 1 to 29, wherein R³ is pyrazol-1-yl, pyrazol-3-yl or pyrazol-4-yl, and wherein pyrazol-1-yl, pyrazol-3-yl and pyrazol-4-yl are optionally substituted with 1, 2 or 3 substituents independently selected from R; and
wherein each R⁷ is independently selected from methyl, cyano, trifluoromethyl, difluoromethyl, trifluoroethyl, ethoxy, methoxy, difluoroethoxy and methylsulfonyl.

31. A compound according to any one of embodiments 1 to 30, wherein
A2 is —O—, —NH— or a bond;
R⁴ is hydrogen, haloalkyl, alkoxyalkyl, dialkylaminoalkyl, cycloalkyl, cycloalkylcarbonyl, aryl, heteroaryl, heteroarylalkyl, heterocycloalkyl or heterocycloalkylalkyl; wherein aryl, cycloalkyl, heteroaryl, heteroarylalkyl, heterocycloalkyl and heterocycloalkylalkyl are optionally substituted with 1, 2 or 3 substituents independently selected from R⁷; if A2 is a bond, then R⁴ can also be halogen or cyano; if A2 is —O— or —NH—, then R⁴ can also be alkyl;
each R¹ is independently selected from alkyl, halogen, cyano, alkylsulfonyl, alkylaminocarbonyl, heterocycloalkyl and alkoxyheterocycloalkylalkyl;
A3 is —O—, —NH— or a bond;
R⁵ is hydrogen, alkylsulfonyl, cycloalkylcarbonyl, heterocycloalkylalkyl, heterocycloalkylalkylcarbonyl, aryl, heteroaryl or heterocycloalkyl, wherein aryl, cycloalkylcarbonyl, heteroaryl and heterocycloalkyl are optionally substituted with 1, 2 or 3 substituents independently selected from R⁹; if A3 is a bond, then R⁵ can also be halogen or cyano; if A3 is —O— or —NH—, then R⁵ can also be alkyl;
each R⁹ is independently selected from alkoxy, halogen, dialkylaminocarbonyl, alkyl, alkoxyalkoxy, alkoxyheterocycloalkylalkyl, alkoxyheterocycloalkylcarbonyl, haloalkyl, haloalkoxy, heterocycloalkylalkoxy, heterocycloalkyl, heterocycloalkyloxy, hydroxy, alkylheterocycloalkyl, alkylheterocycloalkylalkyl, heterocycloalkylalkyl, alkylsulfonyl, (alkyl)heterocycloalkyl, alkylheterocycloalkyloxy, heterocycloalkylheterocycloalkyl, heterocycloalkylheterocycloalkyl, CH₃—O—(CH₂—CH₂—O)ₙ—, alkylaminocarbonyl and cyano; wherein n is selected from 5, 6, 7, 8 and 9; with the proviso that only one of R⁴ and R⁵ can be hydrogen.

32. A compound according to any one of embodiments 1 to 31, wherein A2 is —O— or a bond.

33. A compound according to any one of embodiments 1 to 32, wherein A2 is —O—.

34. A compound according to any one of embodiments 1 to 31, wherein A2 is a bond.

35. A compound according to any one of embodiments 1 to 31, wherein A2 is —NH—.

36. A compound according to any one of embodiments 1 to 35, wherein R⁴ is hydrogen, trifluoromethyl, methoxyethyl, dimethylaminoethyl, cyclopropylcarbonyl, morpholinoethyl, (1,1-dioxo-1,2-thiazolidin-2-yl)methyl, (2-oxopyrrolidin-1-yl)methyl, (2-oxo-1-piperidyl)methyl, (oxetan-3-yl)methyl, phenyl, heteroaryl or heterocycloalkyl, and wherein phenyl, heteroaryl and heterocycloalkyl are optionally substituted with 1, 2 or 3 substituents independently selected from R⁸; if A2 is a bond, then R⁴ can also be fluoro, bromo or cyano; if A2 is —O— or —NH—, then R⁴ can also be methyl;
each R⁸ is independently selected from methyl, fluoro, cyano, 2-oxopyrrolidin-1-yl, methylsulfonyl, oxetan-3-yl and (3-methoxyazetidin-1-yl)methyl.

37. A compound according to any one of embodiments 1 to 36, wherein R⁴ is hydrogen, trifluoromethyl, methoxyethyl, dimethylaminoethyl, cyclopropylcarbonyl, morpholinoethyl, (1,1-dioxo-1,2-thiazolidin-2-yl)methyl, (2-oxopyrrolidin-1-yl)methyl, (2-oxo-1-piperidyl)methyl, heteroaryl or heterocycloalkyl, and wherein heteroaryl and heterocycloalkyl are optionally substituted with 1, 2 or 3 substituents independently selected from R⁸; if A2 is a bond, then R⁴ can also be fluoro, bromo or cyano; if A2 is —O— or —NH—, then R⁴ can also be methyl.

38. A compound according to any one of embodiments 1 to 37, wherein R⁴ is hydrogen, trifluoromethyl, methoxyethyl, dimethylaminoethyl, cyclopropylcarbonyl, morpholinoethyl, (1,1-dioxo-1,2-thiazolidin-2-yl)methyl, (2-oxopyrrolidin-1-yl)methyl, (2-oxo-1-piperidyl)methyl, heteroaryl or heterocycloalkyl, wherein heteroaryl and heterocycloalkyl are optionally substituted with 1, 2 or 3 substituents independently selected from R⁸; if A2 is a bond, then R⁴ can also be fluoro, bromo or cyano; if A2 is —O—, then R⁴ can also be methyl;
each R⁸ is independently selected from methyl, fluoro, cyano, methylsulfonyl, oxetan-3-yl and (3-methoxyazetidin-1-yl)methyl.

39. A compound according to any one of embodiments 1 to 38, wherein the heterocycloalkyl of substituent R⁴ is selected from piperidyl, piperazinyl, pyrrolidinyl, 2,3-dihydropyridazino[4,5-b][1,4]oxazinyl, pyrrolidinyl, 2-oxo-pyrimidinyl, 6,7-dihydro-5H-cyclopenta[c]pyridazin-3-yl, 7-oxo-5H-pyrrolo[3,4-b]pyridin-2-yl, 2-oxa-5-azaspiro[3.4]octanyl and oxetanyl; and
wherein the heteroaryl of substituent R⁴ is selected from 2-oxazol-5-yl, pyridazinyl and pyridyl.

40. A compound according to any one of embodiments 1 to 39, wherein the heterocycloalkyl of substituent R⁴ is selected from 4-piperidyl, piperazin-1-yl, pyrrolidin-3-yl, 2,3-dihydropyridazino[4,5-b][1,4]oxazin-8-yl, pyrrolidin-1-yl, 2-oxo-pyrimidin-4-yl, 2-oxa-5-azaspiro[3.4]octan-5-yl, [rac-(3aS,6aS)-6-oxo-2,3,3a,4,5,6a-hexahydropyrrolo[2,3-c]pyrrol-1-yl] and oxetan-3-yl; and
wherein the heteroaryl of substituent R⁴ is selected from pyridazin-3-yl and 3-pyridyl.

41. A compound according to any one of embodiments 1 to 40, wherein R⁴ is selected from hydrogen, alkoxyalkyl, dialkylaminoalkyl and heterocycloalkyl; if A2 is a bond, then R⁴ can also be halogen; if A2 is —O— or —NH—, then R⁴ can also be alkyl.

42. A compound according to any one of embodiments 1 to 41, wherein R⁴ is selected from hydrogen, methoxyethyl, dimethylaminoethyl and pyrrolidin-3-yl; if A2 is a bond, then R⁴ can also be fluoro; if A2 is —O—, then R⁴ can also be methyl.

43. A compound according to any one of embodiments 1 to 42, wherein R⁴ is selected from methoxyethyl, dimethylaminoethyl and pyrrolidin-3-yl; if A2 is a bond, then R⁴ can also be fluoro; if A2 is —O—, then R⁴ can also be methyl.

44. A compound according to any one of embodiments 1 to 43, wherein R⁴ is hydrogen.

45. A compound according to any one of embodiments 1 to 44, wherein R⁴ is phenyl optionally substituted with one, two or three substituents independently selected from R⁸. 46. A compound according to any one of embodiments 1 to 45, wherein each R⁸ is independently selected from methyl, fluoro, cyano, methylsulfonyl, oxetan-3-yl and (3-methoxyazetidin-1-yl)methyl.

47. A compound according to any one of embodiments 1 to 46, wherein A3 is —O— or —NH—.

48. A compound according to any one of embodiments 1 to 47, wherein A3 is —O—.

49. A compound according to any one of embodiments 1 to 47, wherein A3 is —NH—.

50. A compound according to any one of embodiments 1 to 46, wherein A3 is a bond.

51. A compound according to any one of embodiments 1 to 50, wherein n is 7.

52. A compound according to any one of embodiments 1 to 51, wherein R⁵ is hydrogen, methylsulfonyl, morpholinoethyl, (2-oxopyrrolidin-1-yl)methyl, (2-oxo-1-piperidyl)methyl, (oxetan-3-yl)methyl, 1-piperidylethylcarbonyl, phenyl, heteroaryl or heterocycloalkyl; wherein phenyl, heteroaryl and heterocycloalkyl are optionally substituted with 1, 2 or 3 substituents independently selected from R⁹; if A3 is a bond, then R⁵ can also be fluoro or cyano; if A3 is —O— or —NH—, then R⁵ can also be methyl; and
wherein each R⁹ is independently selected from methyl, methoxy, oxetan-3-yl, azetidin-3-yl, azetidin-1-yl, 3,3-difluoroazetidin-1-yl, 2-oxopyrrolidin-1-yl, hydroxy, difluoromethyl, trifluoromethyl, (3-methoxyazetidin-1-yl)methyl, 3-methoxyazetidine-1-carbonyl, methoxyethoxy, difluoroethoxy, difluoromethoxy, [2-(1,1-dioxo-1,4-thiazinan-4-yl)ethoxy], oxetan-3-yloxy, chloro, dimethylaminocarbonyl, cyano, methylsulfonyl, morpholinomethyl, morpholino, (4-methylpiperazin-1-yl)methyl, CH₃—O—(CH₂—CH₂—O)r-, oxetan-3-yl, [1-(oxetan-3-yl)pyrrolidin-2-yl] and (2-methyl-1,3-dioxolan-2-yl).

53. A compound according to any one of embodiments 1 to 52, wherein R⁵ is hydrogen, methylsulfonyl, morpholinoethyl, (2-oxopyrrolidin-1-yl)methyl, (2-oxo-1-piperidyl)methyl, 1-piperidylethylcarbonyl, heteroaryl or heterocycloalkyl; wherein heteroaryl and heterocycloalkyl are optionally substituted with 1, 2 or 3 substituents independently selected from R⁹; if A3 is a bond, then R⁵ can also be fluoro or cyano; if A3 is —O— or —NH—, then R⁵ can also be methyl.

54. A compound according to any one of embodiments 1 to 53, wherein the heterocycloalkyl of substituent R⁵ is selected from piperazinyl, pyrrolidinyl, piperidyl, 2-oxo-piperidyl, 5-oxo-pyrrolidinyl, (1,1-dioxo-1,2-thiazolidinyl), oxetanyl, 2-oxa-5-azaspiro[3.4]octanyl, 7,8-dihydro-5H-pyrano[4,3-c]pyridazinyl, [rac-(4aS,7aR)-4-methyl-2,3,4a,5,7,7a-hexahydropyrrolo[3,4-b][1,4]oxazinyl] and [rac-(3aS,6aS)-6-oxo-2,3,3a,4,5,6a-hexahydropyrrolo[2,3-c]pyrrolyl]), and wherein the heteroaryl of substituent R⁵ is selected from (pyridazinyl, pyrazolyl, pyridyl, pyrazinyl, pyrimidinyl, (1,3,4-oxadiazolyl), (1,3,4-thiadiazolyl), (1,2,4-triazinyl), isoxazolyl, 2-oxo-pyrimidinyl, 1-methyl-2-oxo-pyridyl, 5,6-dihydropyrrolo[2,3-c]pyridazin-7-yl, 2-oxazol-5-yl, 7-oxo-5H-pyrrolo[3,4-b]pyridin-2-yl and (2,3-dihydropyridazino[4,5-b][1,4]oxazinyl)).

55. A compound according to any one of embodiments 1 to 54, wherein the heterocycloalkyl of substituent R⁵ is selected from piperazin-1-yl, pyrrolidin-1-yl, 4-piperidyl, 2-oxo-4-piperidyl, 5-oxo-pyrrolidin-3-yl, (1,1-dioxo-1,2-thiazolidin-2-yl), oxetan-3-yl, 2-oxa-5-azaspiro[3.4]octan-5-yl, 7,8-dihydro-5H-pyrano[4,3-c]pyridazin-3-yl, [rac-(4aS,7aR)-4-methyl-2,3,4a,5,7,7a-hexahydropyrrolo[3,4-b][1,4]oxazin-6-yl] and [rac-(3aS,6aS)-6-oxo-2,3,3a,4,5,6a-hexahydropyrrolo[2,3-c]pyrrol-1-yl]), and wherein the heteroaryl of substituent R⁵ is selected from (pyridazin-3-yl, pyrazol-4-yl, 3-pyridyl, pyrazin-2-yl, pyrimidin-2-yl, 2-pyridyl, pyrimidin-5-yl, pyridazin-4-yl, (1,3,4-oxadiazol-2-yl), (1,3,4-thiadiazol-2-yl), (1,2,4-triazin-3-yl), isoxazol-3-yl, 2-oxo-pyrimidin-4-yl, 1-methyl-2-oxo-3-pyridyl and (2,3-dihydropyridazino[4,5-b][1,4]oxazin-8-yl)).

56. A compound according to any one of embodiments 1 to 55, wherein R⁵ is pyrimidin-5-yl or pyridazin-3-yl, wherein pyrimidin-5-yl and pyridazin-3-yl are optionally substituted with 1, 2 or 3 substituents independently selected from R⁹.

57. A compound according to any one of embodiments 1 to 56, wherein R⁵ is pyridazin-3-yl, wherein pyridazin-3-yl is optionally substituted with one substituent selected from R⁹.

58. A compound according to any one of embodiments 1 to 57, wherein R⁵ is pyrimidin-5-yl wherein pyrimidin-5-yl is optionally substituted with one substituent selected from R⁹.

59. A compound according to any one of embodiments 1 to 58, wherein R5 is phenyl optionally substituted with one, two or three substituents independently selected from R⁹.

60. A compound according to any one of embodiments 1 to 59, wherein each R⁹ is independently selected from methyl, methoxy, oxetan-3-yl, azetidin-3-yl, hydroxy, difluoromethyl, trifluoromethyl, (3-methoxyazetidin-1-yl)methyl, 3-methoxyazetidine-1-carbonyl, methoxyethoxy, difluoroethoxy, difluoromethoxy, [2-(1,1-dioxo-1,4-thiazinan-4-yl)ethoxy], oxetan-3-yloxy, fluoro, chloro, dimethylaminocarbonyl, (difluoromethyl)cyclopropyl, cyano, methylsulfonyl, morpholinomethyl, morpholino, (4-methylpiperazin-1-yl)methyl, CH₃—O—(CH₂—CH₂—O)r, oxetan-3-yl, [1-(oxetan-3-yl)pyrrolidin-2-yl] and (2-methyl-1,3-dioxolan-2-yl).

61. A compound according to any one of embodiments 1 to 60, wherein each R⁹ is independently selected from methyl, methoxy, difluoromethyl, (3-methoxyazetidin-1-yl)methyl, methoxyethoxy, difluoroethoxy, difluoromethoxy, [2-(1,1-dioxo-1,4-thiazinan-4-yl)ethoxy], oxetan-3-yloxy, dimethylaminocarbonyl, oxetan-3-yl, chloro and trifluoromethyl.

62. A compound according to any one of embodiments 1 to 61, with the proviso that only one of R⁴ and R⁵ can be hydrogen.

63. A compound according to any one of embodiments 1 to 62, with the proviso that only one of R⁴ and R⁵ can be alkyl.

64. A compound according to any one of embodiments 1 to 63, wherein R¹⁰ is hydrogen.

65. A compound according to any one of embodiments 1 to 64, wherein R¹⁰ is alkylcarbonyl.

66. A compound according to any one of embodiments 1 to 65, selected from
[2-(3-chlorophenyl)-6-(5,6-dimethoxybenzimidazol-1-yl)-3-pyridyl]methanol;
(2-(3-chloro-2-fluorophenyl)-6-(5,6-dimethoxy-1H-benzo[d]imidazol-1-yl)pyridin-3-yl)methanol;
(6-(5,6-dimethoxy-1H-benzo[d]imidazol-1-yl)-2-(4-fluoro-2-methoxyphenyl)pyridin-3-yl)methanol;
1-(6-(5,6-dimethoxy-1H-benzo[d]imidazol-1-yl)-2-(4-fluoro-2-methoxyphenyl)pyridin-3-yl)ethan-1-ol;
1-(6-(5,6-dimethoxy-1H-benzo[d]imidazol-1-yl)-2-(4-fluoro-2-methoxyphenyl)pyridin-3-yl)propan-1-ol;
1-(2-(3-chlorophenyl)-6-(5,6-dimethoxy-1H-benzo[d]imidazol-1-yl)pyridin-3-yl)ethan-1-ol;
1-(2-(3-chloro-2-fluorophenyl)-6-(5,6-dimethoxy-1H-benzo[d]imidazol-1-yl)pyridin-3-yl)ethan-1-ol;
(S)-1-(6-(5,6-dimethoxy-1H-benzo[d]imidazol-1-yl)-2-(4-fluoro-2-methoxyphenyl)pyridin-3-yl)ethan-1-ol;
(R)-1-(6-(5,6-dimethoxy-1H-benzo[d]imidazol-1-yl)-2-(4-fluoro-2-methoxyphenyl)pyridin-3-yl)ethan-1-ol;
cyclopropyl(6-(5,6-dimethoxy-1H-benzo[d]imidazol-1-yl)-2-(4-fluoro-2-methoxyphenyl)pyridin-3-yl)methanol;
1-(2-(3-chlorophenyl)-6-(5,6-dimethoxy-1H-benzo[d]imidazol-1-yl)pyridin-3-yl)propan-1-ol;
(2-(3-chlorophenyl)-6-(5,6-dimethoxy-1H-benzo[d]imidazol-1-yl)pyridin-3-yl)(cyclopropyl)methanol;
(6-(5,6-dimethoxy-1H-benzo[d]imidazol-1-yl)-2-morpholinopyridin-3-yl)methanol;
(6-(5,6-dimethoxy-1H-benzo[d]imidazol-1-yl)-2-(pyrrolidin-1-yl)pyridin-3-yl)methanol;
(6-(5,6-dimethoxy-1H-benzo[d]imidazol-1-yl)-2-(piperidin-1-yl)pyridin-3-yl)methanol;
5-((6-(5,6-dimethoxy-1H-benzo[d]imidazol-1-yl)-3-(hydroxymethyl)pyridin-2-yl)amino)pentan-1-ol;
1-(6-(5,6-dimethoxy-1H-benzo[d]imidazol-1-yl)-3-(hydroxymethyl)pyridin-2-yl)pyrrolidin-2-one;
4-((6-(5,6-dimethoxy-1H-benzo[d]imidazol-1-yl)-3-(hydroxymethyl)pyridin-2-yl)amino)butan-1-ol;
(S)-2-(6-(5,6-dimethoxy-1H-benzo[d]imidazol-1-yl)-3-(1-hydroxyethyl)pyridin-2-yl)benzamide;
(R)-2-(6-(5,6-dimethoxy-1H-benzo[d]imidazol-1-yl)-3-(1-hydroxyethyl)pyridin-2-yl)benzamide;
(S)-3-(6-(5,6-dimethoxy-1H-benzo[d]imidazol-1-yl)-3-(1-hydroxyethyl)pyridin-2-yl)benzonitrile;
(R)-3-(6-(5,6-dimethoxy-1H-benzo[d]imidazol-1-yl)-3-(1-hydroxyethyl)pyridin-2-yl)benzonitrile;
(S)-4-(6-(5,6-dimethoxy-1H-benzo[d]imidazol-1-yl)-3-(1-hydroxyethyl)pyridin-2-yl)benzonitrile;
(R)-4-(6-(5,6-dimethoxy-1H-benzo[d]imidazol-1-yl)-3-(1-hydroxyethyl)pyridin-2-yl)benzonitrile;
(S)-6-(5,6-dimethoxy-1H-benzo[d]imidazol-1-yl)-3-(1-hydroxyethyl)-1′-methyl-[2,4′-bipyridin]-2′(1′H)-one;
(R)-6-(5,6-dimethoxy-1H-benzo[d]imidazol-1-yl)-3-(1-hydroxyethyl)-1′-methyl-[2,4′-bipyridin]-2′(1′H)-one;
1-[6-[5-(2-morpholinoethoxy)benzimidazol-1-yl]-2-phenoxy-3-pyridyl]ethanol;
1-[2-anilino-6-[5-(2-morpholinoethoxy)benzimidazol-1-yl]-3-pyridyl]ethanol;
1-[6-[5-(2-morpholinoethoxy)benzimidazol-1-yl]-2-[3-(trifluoromethyl)pyrazol-1-yl]-3-pyridyl]ethanol;
1-[6-(5,6-dimethoxybenzimidazol-1-yl)-2-[3-(trifluoromethyl)pyrazol-1-yl]-3-pyridyl]ethanol;
1-[3-(1-hydroxyethyl)-6-[5-[(6-methylpyridazin-3-yl)amino]benzimidazol-1-yl]-2-pyridyl]-5-methyl-pyrazole-3-carbonitrile;
1-[3-(1-hydroxyethyl)-6-[5-(4-methylpiperazin-1-yl)benzimidazol-1-yl]-2-pyridyl]-5-methyl-pyrazole-3-carbonitrile;
1-[3-(1-hydroxyethyl)-6-[5-[4-(oxetan-3-yl)piperazin-1-yl]benzimidazol-1-yl]-2-pyridyl]-5-methyl-pyrazole-3-carbonitrile;
1-[3-(1-hydroxyethyl)-6-[5-(2-morpholinoethoxy)benzimidazol-1-yl]-2-pyridyl]-5-methyl-pyrazole-3-carbonitrile;
1-[3-(1-hydroxyethyl)-6-[5-[[1-(oxetan-3-yl)pyrazol-4-yl]amino]benzimidazol-1-yl]-2-pyridyl]-5-methyl-pyrazole-3-carbonitrile;
1-[6-[5-[[1-(azetidin-3-yl)pyrazol-4-yl]amino]benzimidazol-1-yl]-3-(1-hydroxyethyl)-2-pyridyl]-5-methyl-pyrazole-3-carbonitrile;
1-[3-(1-hydroxyethyl)-6-[6-(6-methylpyridazin-3-yl)oxybenzimidazol-1-yl]-2-pyridyl]-5-methyl-pyrazole-3-carbonitrile;
1-[3-(1-hydroxyethyl)-6-[5-(6-methylpyridazin-3-yl)oxybenzimidazol-1-yl]-2-pyridyl]-5-methyl-pyrazole-3-carbonitrile;
5-[3-(1-hydroxyethyl)-6-[5-[(6-methylpyridazin-3-yl)amino]benzimidazol-1-yl]-2-pyridyl]-1-methyl-pyrazole-4-carbonitrile;
1-[3-(1-hydroxyethyl)-6-[5-[rac-(3R)-3-hydroxypyrrolidin-1-yl]benzimidazol-1-yl]-2-pyridyl]-5-methyl-pyrazole-3-carbonitrile;
1-[3-(1-hydroxyethyl)-6-[5-[(3S)-3-hydroxypyrrolidin-1-yl]benzimidazol-1-yl]-2-pyridyl]-5-methyl-pyrazole-3-carbonitrile;
1-[3-(1-hydroxyethyl)-6-[5-[(6-methyl-3-pyridyl)amino]benzimidazol-1-yl]-2-pyridyl]-5-methyl-pyrazole-3-carbonitrile;
1-[3-(1-hydroxyethyl)-6-[5-[(5-methylpyrazin-2-yl)amino]benzimidazol-1-yl]-2-pyridyl]-5-methyl-pyrazole-3-carbonitrile;
1-[3-(1-hydroxyethyl)-6-[5-[(5-methylpyrimidin-2-yl)amino]benzimidazol-1-yl]-2-pyridyl]-5-methyl-pyrazole-3-carbonitrile;
1-[3-(1-hydroxyethyl)-6-[5-[(5-methyl-2-pyridyl)amino]benzimidazol-1-yl]-2-pyridyl]-5-methyl-pyrazole-3-carbonitrile;
1-[2-(1,1-dioxo-1,2-thiazolidin-2-yl)-6-[5-[(6-methylpyridazin-3-yl)amino]benzimidazol-1-yl]-3-pyridyl]ethanol;
1-[3-(1-hydroxyethyl)-6-[5-[(2-methylpyrimidin-5-yl)amino]benzimidazol-1-yl]-2-pyridyl]-5-methyl-pyrazole-3-carbonitrile;
1-[3-(1-hydroxyethyl)-6-[5-(pyridazin-4-ylamino)benzimidazol-1-yl]-2-pyridyl]-5-methyl-pyrazole-3-carbonitrile;
1-[3-(1-hydroxyethyl)-6-[5-[(6-methoxypyridazin-3-yl)amino]benzimidazol-1-yl]-2-pyridyl]-5-methyl-pyrazole-3-carbonitrile;
1-[3-(1-hydroxyethyl)-6-[5-(pyridazin-3-ylamino)benzimidazol-1-yl]-2-pyridyl]-5-methyl-pyrazole-3-carbonitrile;
1-[6-[5-[[6-(difluoromethyl)pyridazin-3-yl]amino]benzimidazol-1-yl]-3-(1-hydroxyethyl)-2-pyridyl]-5-methyl-pyrazole-3-carbonitrile;
1-[6-[6-fluoro-5-[(6-methylpyridazin-3-yl)amino]benzimidazol-1-yl]-3-(1-hydroxyethyl)-2-pyridyl]-5-methyl-pyrazole-3-carbonitrile;
1-[6-[5-fluoro-6-[(6-methylpyridazin-3-yl)amino]benzimidazol-1-yl]-3-(1-hydroxyethyl)-2-pyridyl]-5-methyl-pyrazole-3-carbonitrile;
1-[6-[5-[(6-methylpyridazin-3-yl)amino]benzimidazol-1-yl]-2-[3-(trifluoromethyl)-4,5,6,7-tetrahydropyrazolo[4,3-c]pyridin-1-yl]-3-pyridyl]ethanol;
1-[6-[6-[(6-methylpyridazin-3-yl)amino]benzimidazol-1-yl]-2-[3-(trifluoromethyl)-4,5,6,7-tetrahydropyrazolo[4,3-c]pyridin-1-yl]-3-pyridyl]ethanol;
1-[3-(1-hydroxyethyl)-6-[5-[(5-methyl-1,3,4-oxadiazol-2-yl)amino]benzimidazol-1-yl]-2-pyridyl]-5-methyl-pyrazole-3-carbonitrile;
2-[3-(1-hydroxyethyl)-6-[5-[(6-methylpyridazin-3-yl)amino]benzimidazol-1-yl]-2-pyridyl]-5-methyl-pyrazole-3-carbonitrile;
(3R)-1-[3-(1-hydroxyethyl)-6-[6-[(6-methylpyridazin-3-yl)amino]benzimidazol-1-yl]-2-pyridyl]pyrrolidine-3-carbonitrile;
rac-(3R)-1-[3-(1-hydroxyethyl)-6-[5-[(6-methylpyridazin-3-yl)amino]benzimidazol-1-yl]-2-pyridyl]pyrrolidine-3-carbonitrile;
1-[2-(3,5-dimethylpyrazol-1-yl)-6-[6-[(6-methylpyridazin-3-yl)amino]benzimidazol-1-yl]-3-pyridyl]ethanol;
1-[2-(3,5-dimethylpyrazol-1-yl)-6-[5-[(6-methylpyridazin-3-yl)amino]benzimidazol-1-yl]-3-pyridyl]ethanol;
5-methyl-1-[6-[5-[(6-methylpyridazin-3-yl)amino]benzimidazol-1-yl]-3-[rac-(1R)-1-hydroxyethyl]-2-pyridyl]pyrazole-3-carbonitrile;
5-methyl-1-[6-[5-[(6-methylpyridazin-3-yl)amino]benzimidazol-1-yl]-3-[rac-(1S)-1-hydroxyethyl]-2-pyridyl]pyrazole-3-carbonitrile;
1-[3-(1-hydroxyethyl)-6-[5-[(5-methyl-1,3,4-thiadiazol-2-yl)amino]benzimidazol-1-yl]-2-pyridyl]-5-methyl-pyrazole-3-carbonitrile;
1-[3-(1-hydroxyethyl)-6-[5-[[1-(oxetan-3-yl)-4-piperidyl]oxy]benzimidazol-1-yl]-2-pyridyl]-5-methyl-pyrazole-3-carbonitrile;
1-[3-(1-hydroxyethyl)-6-[6-[[1-(oxetan-3-yl)-4-piperidyl]oxy]benzimidazol-1-yl]-2-pyridyl]-5-methyl-pyrazole-3-carbonitrile;
1-[3-(1-hydroxyethyl)-6-[5-[(6-methylpyridazin-3-yl)amino]benzimidazol-1-yl]-2-pyridyl]pyrazole-3-carbonitrile;
1-[[[3-(1-hydroxyethyl)-6-[5-[(6-methylpyridazin-3-yl)amino]benzimidazol-1-yl]-2-pyridyl]amino]methyl]cyclopropanecarbonitrile;
1-[[[3-(1-hydroxyethyl)-6-[6-[(6-methylpyridazin-3-yl)amino]benzimidazol-1-yl]-2-pyridyl]amino]methyl]cyclopropanecarbonitrile;
5-[3-(1-hydroxyethyl)-6-[5-[(6-methylpyridazin-3-yl)amino]benzimidazol-1-yl]-2-pyridyl]pyridine-3-carbonitrile;
1-[3-(1-hydroxyethyl)-6-[5-(1,2,4-triazin-3-ylamino)benzimidazol-1-yl]-2-pyridyl]-5-methyl-pyrazole-3-carbonitrile;
1-[3-(1-hydroxyethyl)-6-[6-[(6-methylpyridazin-3-yl)amino]benzimidazol-1-yl]-2-pyridyl]-3-methyl-pyrrolidine-3-carbonitrile;
1-[3-(1-hydroxyethyl)-6-[5-[(6-methylpyridazin-3-yl)amino]benzimidazol-1-yl]-2-pyridyl]-3-methyl-pyrrolidine-3-carbonitrile;
1-[6-[5-[(6-methylpyridazin-3-yl)amino]benzimidazol-1-yl]-2-[2-methyl-5-(trifluoromethyl)pyrazol-3-yl]-3-pyridyl]ethanol;
1-[3-(1-hydroxyethyl)-6-[5-(isoxazol-3-ylamino)benzimidazol-1-yl]-2-pyridyl]-5-methyl-pyrazole-3-carbonitrile;
3-(1-hydroxyethyl)-6-[5-[(6-methylpyridazin-3-yl)amino]benzimidazol-1-yl]-N-(2,2,2-trifluoroethyl)pyridine-2-carboxamide;
1-[6-[6-[(6-methylpyridazin-3-yl)amino]benzimidazol-1-yl]-2-[rac-(3aR,6aS)-2,3,3a,5,6,6a-hexahydro-1H-pyrrolo[3,2-b]pyrrol-4-yl]-3-pyridyl]ethanol;
1-[6-[6-[(6-methylpyridazin-3-yl)amino]benzimidazol-1-yl]-2-[rac-(3aS,6aR)-2,3,3a,5,6,6a-hexahydro-1H-pyrrolo[3,2-b]pyrrol-4-yl]-3-pyridyl]ethanol;
1-[6-[5-[(6-methylpyridazin-3-yl)amino]benzimidazol-1-yl]-2-[rac-(3aR,6aS)-2,3,3a,5,6,6a-hexahydro-1H-pyrrolo[3,2-b]pyrrol-4-yl]-3-pyridyl]ethanol;
1-[2-[3-(difluoromethyl)-5-methyl-pyrazol-1-yl]-6-[6-[(6-methylpyridazin-3-yl)amino]benzimidazol-1-yl]-3-pyridyl]ethanol;
1-[2-[3-(difluoromethyl)-5-methyl-pyrazol-1-yl]-6-[5-[(6-methylpyridazin-3-yl)amino]benzimidazol-1-yl]-3-pyridyl]ethanol;
1-[6-[6-bromo-5-[4-(oxetan-3-yl)piperazin-1-yl]benzimidazol-1-yl]-3-(1-hydroxyethyl)-2-pyridyl]-5-methyl-pyrazole-3-carbonitrile;
1-[2-(3-ethoxy-5-methyl-pyrazol-1-yl)-6-[5-[(6-methylpyridazin-3-yl)amino]benzimidazol-1-yl]-3-pyridyl]ethanol;
1-[2-(3-ethoxy-5-methyl-pyrazol-1-yl)-6-[6-[(6-methylpyridazin-3-yl)amino]benzimidazol-1-yl]-3-pyridyl]ethanol;
1-[2-(6-methylpyridazin-4-yl)-6-[5-[(6-methylpyridazin-3-yl)amino]benzimidazol-1-yl]-3-pyridyl]ethanol; 4-[3-(1-hydroxyethyl)-6-[5-[(6-methylpyridazin-3-yl)amino]benzimidazol-1-yl]-2-pyridyl]-1-(2,2,2-trifluoroethyl)piperazin-2-one; 4-[3-(1-hydroxyethyl)-6-[6-[(6-methylpyridazin-3-yl)amino]benzimidazol-1-yl]-2-pyridyl]-1-(2,2,2-trifluoroethyl)piperazin-2-one;
1-[[3-(1-hydroxyethyl)-6-[6-[(6-methylpyridazin-3-yl)amino]benzimidazol-1-yl]-2-pyridyl]oxymethyl]cyclopropanecarbonitrile;
1-[[3-(1-hydroxyethyl)-6-[5-[(6-methylpyridazin-3-yl)amino]benzimidazol-1-yl]-2-pyridyl]oxymethyl]cyclopropanecarbonitrile;
1-[3-(1-hydroxyethyl)-6-[5-methoxy-6-[(6-methylpyridazin-3-yl)amino]benzimidazol-1-yl]-2-pyridyl]-5-methyl-pyrazole-3-carbonitrile;
1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanol;
1-[3-(1-hydroxyethyl)-6-[6-[(6-methylpyridazin-3-yl)amino]benzimidazol-1-yl]-2-pyridyl]-N,5-dimethyl-pyrazole-3-carboxamide;
1-[2-(5-methyl-3-methylsulfonyl-pyrazol-1-yl)-6-[5-[(6-methylpyridazin-3-yl)amino]benzimidazol-1-yl]-3-pyridyl]ethanol;
1-[3-(1-hydroxyethyl)-6-[5-[(2-keto-1-methyl-pyrimidin-4-yl)amino]benzimidazol-1-yl]-2-pyridyl]-5-methyl-pyrazole-3-carbonitrile;
5-methyl-1-[6-[5-[(6-methylpyridazin-3-yl)amino]benzimidazol-1-yl]-3-(2,2,2-trifluoro-1-hydroxy-ethyl)-2-pyridyl]pyrazole-3-carbonitrile;
1-[2-[1-(difluoromethyl)-3-methyl-pyrazol-4-yl]-6-[5-[(6-methylpyridazin-3-yl)amino]benzimidazol-1-yl]-3-pyridyl]ethanol;
1-[3-(1-hydroxyethyl)-6-[5-[(6-methylpyridazin-3-yl)amino]benzimidazol-1-yl]-2-pyridyl]-5-methyl-pyrazole-4-carbonitrile;
1-[3-(1-hydroxyethyl)-6-[6-[(6-methylpyridazin-3-yl)amino]benzimidazol-1-yl]-2-pyridyl]-5-methyl-pyrazole-4-carbonitrile;
1-[3-(1-hydroxyethyl)-6-[5-[(6-methylpyridazin-3-yl)amino]benzimidazol-1-yl]-2-pyridyl]-N,5-dimethyl-pyrazole-3-carboxamide;
1-[3-(1-hydroxyethyl)-6-[5-[(2-keto-1-methyl-4-piperidyl)amino]benzimidazol-1-yl]-2-pyridyl]-5-methyl-pyrazole-3-carbonitrile; [2-(3-methoxy-5-methyl-pyrazol-1-yl)-6-[5-[(6-methylpyridazin-3-yl)amino]benzimidazol-1-yl]-3-pyridyl]methanol;
1-[2-(3,5-dimethylisoxazol-4-yl)-6-[5-[(6-methylpyridazin-3-yl)amino]benzimidazol-1-yl]-3-pyridyl]ethanol;
1-[3-(1-hydroxyethyl)-6-[5-[(4-methoxypyridazin-3-yl)amino]benzimidazol-1-yl]-2-pyridyl]-5-methyl-pyrazole-3-carbonitrile;
1-[3-(1-hydroxyethyl)-6-[5-[(5-keto-1-methyl-pyrrolidin-3-yl)amino]benzimidazol-1-yl]-2-pyridyl]-5-methyl-pyrazole-3-carbonitrile;
1-[6-[5-[(6-methylpyridazin-3-yl)amino]benzimidazol-1-yl]-2-(3-methyl-4-pyridyl)-3-pyridyl]ethanol;
1-[2-[4-(cyclopropylamino)pyrimidin-5-yl]-6-[5-[(6-methylpyridazin-3-yl)amino]benzimidazol-1-yl]-3-pyridyl]ethanol;
5-[3-(1-hydroxyethyl)-6-[5-[(6-methylpyridazin-3-yl)amino]benzimidazol-1-yl]-2-pyridyl]-4-(trifluoromethyl)pyridin-2-ol;
3-[3-(1-hydroxyethyl)-6-[5-[(6-methylpyridazin-3-yl)amino]benzimidazol-1-yl]-2-pyridyl]-6,7-dihydro-5H-pyrazolo[1,5-a]pyrazin-4-one;
1-[6-[6-fluoro-5-[[(3S,4R)-4-fluoropyrrolidin-3-yl]amino]benzimidazol-1-yl]-3-(1-hydroxyethyl)-2-pyridyl]-5-methyl-pyrazole-3-carbonitrile;
1-[2-[2-(difluoromethoxy)-5-methyl-4-pyridyl]-6-[5-[(6-methylpyridazin-3-yl)amino]benzimidazol-1-yl]-3-pyridyl]ethanol;
1-[2-(2-fluoro-5-methyl-4-pyridyl)-6-[5-[(6-methylpyridazin-3-yl)amino]benzimidazol-1-yl]-3-pyridyl]ethanol;
1-[3-(1-hydroxyethyl)-6-[6-[(6-methylpyridazin-3-yl)amino]benzimidazol-1-yl]-2-pyridyl]-4-methyl-pyrazole-3-carbonitrile;
1-[3-(1-hydroxyethyl)-6-[5-[(6-methylpyridazin-3-yl)amino]benzimidazol-1-yl]-2-pyridyl]-4-methyl-pyrazole-3-carbonitrile;
1-[6-[5-[(6-methylpyridazin-3-yl)amino]benzimidazol-1-yl]-2-[5-methyl-3-(trifluoromethyl)-6,7-dihydro-4H-pyrazolo[4,3-c]pyridin-1-yl]-3-pyridyl]ethanol;
1-[[[3-(1-hydroxyethyl)-6-[6-[(6-methylpyridazin-3-yl)amino]benzimidazol-1-yl]-2-pyridyl]-methyl-amino]methyl]cyclopropanecarbonitrile;
1-[[[3-(1-hydroxyethyl)-6-[5-[(6-methylpyridazin-3-yl)amino]benzimidazol-1-yl]-2-pyridyl]-methyl-amino]methyl]cyclopropanecarbonitrile;
1-[2-(2-chloro-5-fluoro-3-pyridyl)-6-[5-[(6-methylpyridazin-3-yl)amino]benzimidazol-1-yl]-3-pyridyl]ethanol;
1-[3-(1-hydroxyethyl)-6-[6-[[6-[(3-methoxyazetidin-1-yl)methyl]pyridazin-3-yl]amino]benzimidazol-1-yl]-2-pyridyl]-5-methyl-pyrazole-3-carbonitrile;
1-[3-(1-hydroxyethyl)-6-[5-[[6-[(3-methoxyazetidin-1-yl)methyl]pyridazin-3-yl]amino]benzimidazol-1-yl]-2-pyridyl]-5-methyl-pyrazole-3-carbonitrile;
1-[6-[5-[(6-methylpyridazin-3-yl)amino]benzimidazol-1-yl]-2-[5-(oxetan-3-yl)-3-(trifluoromethyl)-6,7-dihydro-4H-pyrazolo[4,3-c]pyridin-1-yl]-3-pyridyl]ethanol;
1-[3-(1-hydroxyethyl)-6-[6-[(6-methylpyridazin-3-yl)amino]benzimidazol-1-yl]-2-pyridyl]-3-methyl-pyrazole-4-carbonitrile;
1-[2-[2-(methylamino)-3-pyridyl]-6-[5-[(6-methylpyridazin-3-yl)amino]benzimidazol-1-yl]-3-pyridyl]ethanol;
1-[3-(1-hydroxyethyl)-6-[5-[(6-methylpyridazin-3-yl)amino]benzimidazol-1-yl]-2-pyridyl]-3-methyl-pyrazole-4-carbonitrile;
1-[3-(1-hydroxyethyl)-6-[5-[(6-methylpyridazin-3-yl)amino]benzimidazol-1-yl]-2-pyridyl]-3-methyl-azetidine-3-carbonitrile;
1-[3-(1-hydroxyethyl)-6-[6-[(6-methylpyridazin-3-yl)amino]benzimidazol-1-yl]-2-pyridyl]-3-methyl-azetidine-3-carbonitrile;
1-[3-(1-hydroxyethyl)-6-[6-methoxy-5-[(6-methylpyridazin-3-yl)amino]benzimidazol-1-yl]-2-pyridyl]-5-methyl-pyrazole-3-carbonitrile;
1-[6-[5-[(6-methylpyridazin-3-yl)amino]benzimidazol-1-yl]-2-[2-(1H-pyrazol-4-yl)-3-pyridyl]-3-pyridyl]ethanol;
1-[3-(1-hydroxyethyl)-6-[6-(2-methoxyethoxy)-5-[(6-methylpyridazin-3-yl)amino]benzimidazol-1-yl]-2-pyridyl]-5-methyl-pyrazole-3-carbonitrile;
1-[6-[5-fluoro-6-[[rac-(3R,4S)-4-fluoropyrrolidin-3-yl]amino]benzimidazol-1-yl]-3-(1-hydroxyethyl)pyridin-2-yl]-5-methylpyrazole-3-carbonitrile;
1-[6-[6-[2-(dimethylamino)ethoxy]-5-[(6-methylpyridazin-3-yl)amino]benzimidazol-1-yl]-3-(1-hydroxyethyl)-2-pyridyl]-5-methyl-pyrazole-3-carbonitrile;
1-[6-[5-bromo-6-[4-(oxetan-3-yl)piperazino]benzimidazol-1-yl]-3-(1-hydroxyethyl)-2-pyridyl]-5-methyl-pyrazole-3-carbonitrile;
1-[3-(1-hydroxyethyl)-6-[5-[[6-(3-methoxyazetidine-1-carbonyl)pyridazin-3-yl]amino]benzimidazol-1-yl]-2-pyridyl]-5-methyl-pyrazole-3-carbonitrile;
1-[3-(1-hydroxyethyl)-6-[5-[(6-methylpyridazin-3-yl)amino]-6-pyrrolidin-3-yloxy-benzimidazol-1-yl]-2-pyridyl]-5-methyl-pyrazole-3-carbonitrile;
1-[3-(1-hydroxyethyl)-6-[5-[[6-(2-methoxyethoxy)pyridazin-3-yl]amino]benzimidazol-1-yl]-2-pyridyl]-5-methyl-pyrazole-3-carbonitrile;
1-[2-(1H-benzotriazol-4-yl)-6-[5-[(6-methylpyridazin-3-yl)amino]benzimidazol-1-yl]-3-pyridyl]ethanol;
1-[6-[5-[[6-(2,2-difluoroethoxy)pyridazin-3-yl]amino]benzimidazol-1-yl]-3-(1-hydroxyethyl)-2-pyridyl]-5-methyl-pyrazole-3-carbonitrile;
1-[6-[5-[[6-(difluoromethoxy)pyridazin-3-yl]amino]benzimidazol-1-yl]-3-(1-hydroxyethyl)-2-pyridyl]-5-methyl-pyrazole-3-carbonitrile;
1-[3-(2,2-difluoro-1-hydroxy-ethyl)-6-[5-[(6-methylpyridazin-3-yl)amino]benzimidazol-1-yl]-2-pyridyl]-5-methyl-pyrazole-3-carbonitrile;
1-[3-(2,2-difluoro-1-hydroxy-ethyl)-6-[6-[(6-methylpyridazin-3-yl)amino]benzimidazol-1-yl]-2-pyridyl]-5-methyl-pyrazole-3-carbonitrile;
1-[6-[6-[(1,1-dioxo-1,2-thiazolidin-2-yl)methyl]benzimidazol-1-yl]-3-(1-hydroxyethyl)-2-pyridyl]-5-methyl-pyrazole-3-carbonitrile;
1-[6-[5-[(1,1-dioxo-1,2-thiazolidin-2-yl)methyl]benzimidazol-1-yl]-3-(1-hydroxyethyl)-2-pyridyl]-5-methyl-pyrazole-3-carbonitrile;
3-[3-(1-hydroxyethyl)-6-[5-[(6-methylpyridazin-3-yl)amino]benzimidazol-1-yl]-2-pyridyl]-2-methoxy-phenol;
3-[3-(1-hydroxyethyl)-6-[5-[(6-methylpyridazin-3-yl)amino]benzimidazol-1-yl]-2-pyridyl]furan-2-carbonitrile;
1-[3-(1-hydroxyethyl)-6-[5-[(6-methylpyridazin-3-yl)amino]benzimidazol-1-yl]pyridin-2-yl]-4,5,6,7-tetrahydropyrazolo[4,3-c]pyridine-3-carbonitrile;
1-[6-[5-[[6-[2-(1,1-diketo-1,4-thiazinan-4-yl)ethoxy]pyridazin-3-yl]amino]benzimidazol-1-yl]-3-(1-hydroxyethyl)-2-pyridyl]-5-methyl-pyrazole-3-carbonitrile;
1-[3-(1-hydroxyethyl)-6-[5-[[6-(oxetan-3-yloxy)pyridazin-3-yl]amino]benzimidazol-1-yl]-2-pyridyl]-5-methyl-pyrazole-3-carbonitrile;
1-[6-[5-[(6-methylpyridazin-3-yl)amino]benzimidazol-1-yl]-2-[(3S)-pyrrolidin-3-yl]oxy-3-pyridyl]ethanol;
1-[3-(1-hydroxyethyl)-6-[6-[(2-oxopyrrolidin-1-yl)methyl]benzimidazol-1-yl]-2-pyridyl]-5-methyl-pyrazole-3-carbonitrile;
1-[3-(1-hydroxyethyl)-6-[5-[(2-oxopyrrolidin-1-yl)methyl]benzimidazol-1-yl]-2-pyridyl]-5-methyl-pyrazole-3-carbonitrile;
1-[2-(3-methoxy-5-methyl-pyrazol-1-yl)-6-[5-[(6-methylpyridazin-3-yl)amino]benzimidazol-1-yl]-3-pyridyl]ethanol;
1-[3-(1-hydroxyethyl)-6-[6-[(2-oxo-1-piperidyl)methyl]benzimidazol-1-yl]-2-pyridyl]-5-methyl-pyrazole-3-carbonitrile;
1-[3-(1-hydroxyethyl)-6-[5-[(2-oxo-1-piperidyl)methyl]benzimidazol-1-yl]-2-pyridyl]-5-methyl-pyrazole-3-carbonitrile; 3-[[1-[6-(3-cyano-5-methyl-pyrazol-1-yl)-5-(1-hydroxyethyl)-2-pyridyl]benzimidazol-5-yl]amino]-N,N,6-trimethyl-pyridazine-4-carboxamide; 5-hydroxy-2-[3-(1-hydroxyethyl)-6-[5-[(6-methylpyridazin-3-yl)amino]benzimidazol-1-yl]-2-pyridyl]benzonitrile;
5-[3-(1-hydroxyethyl)-6-[5-[(6-methylpyridazin-3-yl)amino]benzimidazol-1-yl]-2-pyridyl]-3-(trifluoromethyl)-1H-pyridazin-6-one;
2-[3-(1-hydroxyethyl)-6-[5-[(6-methylpyridazin-3-yl)amino]benzimidazol-1-yl]-2-pyridyl]furan-3-carbonitrile;
1-[2-[2-(2,2-difluorocyclopropyl)pyrazol-3-yl]-6-[5-[(6-methylpyridazin-3-yl)amino]benzimidazol-1-yl]-3-pyridyl]ethanol;
1-[3-(1-hydroxyethyl)-6-[5-[[6-(oxetan-3-yl)pyridazin-3-yl]amino]benzimidazol-1-yl]-2-pyridyl]-5-methyl-pyrazole-3-carbonitrile; (1S)-1-[2-[3-(difluoromethyl)-5-methyl-pyrazol-1-yl]-6-[5-[(6-methylpyridazin-3-yl)amino]benzimidazol-1-yl]-3-pyridyl]ethanol;
(1R)-1-[2-[3-(difluoromethyl)-5-methyl-pyrazol-1-yl]-6-[5-[(6-methylpyridazin-3-yl)amino]benzimidazol-1-yl]-3-pyridyl]ethanol;
1-[2-[5-(difluoromethyl)-3-methyl-pyrazol-1-yl]-6-[5-[(6-methylpyridazin-3-yl)amino]benzimidazol-1-yl]-3-pyridyl]ethanol;
1-[3-(1-hydroxy-2-methoxy-ethyl)-6-[5-[(6-methylpyridazin-3-yl)amino]benzimidazol-1-yl]-2-pyridyl]-5-methyl-pyrazole-3-carbonitrile;
1-[3-(1-hydroxy-2-methoxy-ethyl)-6-[6-[(6-methylpyridazin-3-yl)amino]benzimidazol-1-yl]-2-pyridyl]-5-methyl-pyrazole-3-carbonitrile;
1-[6-[6-fluoro-5-[(6-methylpyridazin-3-yl)amino]benzimidazol-1-yl]-3-[(1S)-1-hydroxyethyl]-2-pyridyl]-5-methyl-pyrazole-3-carbonitrile;
1-[6-[6-fluoro-5-[(6-methylpyridazin-3-yl)amino]benzimidazol-1-yl]-3-[(1R)-1-hydroxyethyl]-2-pyridyl]-5-methyl-pyrazole-3-carbonitrile;
1-[5-chloro-3-(1-hydroxyethyl)-6-[5-[(6-methylpyridazin-3-yl)amino]benzimidazol-1-yl]-2-pyridyl]pyrazole-3-carbonitrile;
1-[6-[5-[(6-methylpyridazin-3-yl)amino]benzimidazol-1-yl]-2-[5-methyl-1-(2,2,2-trifluoroethyl)pyrazol-4-yl]-3-pyridyl]ethanol;
1-[6-[5-[(6-chloro-4-methoxy-pyridazin-3-yl)amino]benzimidazol-1-yl]-3-(1-hydroxyethyl)-2-pyridyl]-5-methyl-pyrazole-3-carbonitrile;
rac-(3R,5S)-1-[3-(1-hydroxyethyl)-6-[5-[(6-methylpyridazin-3-yl)amino]benzimidazol-1-yl]-2-pyridyl]-5-methyl-pyrrolidine-3-carbonitrile;
1-[5-chloro-3-(1-hydroxyethyl)-6-[5-[(6-methylpyridazin-3-yl)amino]benzimidazol-1-yl]-2-pyridyl]-5-methyl-pyrazole-3-carbonitrile;
5-[3-(1-hydroxyethyl)-6-[5-[(6-methylpyridazin-3-yl)amino]benzimidazol-1-yl]-2-pyridyl]-2-methyl-pyrazole-3-carbonitrile;
1-[3-[(1R)-1-hydroxyethyl]-6-[6-[(6-methylpyridazin-3-yl)amino]benzimidazol-1-yl]-2-pyridyl]-5-methyl-pyrazole-3-carbonitrile;
1-[2-[3,5-bis(difluoromethyl)pyrazol-1-yl]-6-[5-[(6-methylpyridazin-3-yl)amino]benzimidazol-1-yl]pyridin-3-yl]ethanol;
1-[3-(1-hydroxyethyl)-6-[5-[[6-(trifluoromethyl)pyridazin-3-yl]amino]benzimidazol-1-yl]-2-pyridyl]-5-methyl-pyrazole-3-carbonitrile;
1-[2-[2-ethyl-5-(trifluoromethyl)pyrazol-3-yl]-6-[5-[(6-methylpyridazin-3-yl)amino]benzimidazol-1-yl]-3-pyridyl]ethanol;
rac-(1S)-1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanol;
rac-(1R)-1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanol;
1-[3-(1-hydroxyethyl)-6-[5-[(6-methylpyridazin-3-yl)amino]benzimidazol-1-yl]-2-pyridyl]triazole-4-carbonitrile;
(3S,5R)-1-[3-(1-hydroxyethyl)-6-[5-[(6-methylpyridazin-3-yl)amino]benzimidazol-1-yl]-2-pyridyl]-5-methyl-pyrrolidine-3-carbonitrile;
5-[3-(1-hydroxyethyl)-6-[5-[(6-methylpyridazin-3-yl)amino]benzimidazol-1-yl]-2-pyridyl]-1-methyl-pyrazole-3-carbonitrile;
2-[3-(1-hydroxyethyl)-6-[5-[(6-methylpyridazin-3-yl)amino]benzimidazol-1-yl]-2-pyridyl]triazole-4-carbonitrile;
1-[6-(3-cyano-5-methylpyrazol-1-yl)-5-(1-hydroxyethyl)pyridin-2-yl]benzimidazole-5-carbonitrile;
1-[2-(1-ethyl-3-methyl-pyrazol-4-yl)-6-[5-[(6-methylpyridazin-3-yl)amino]benzimidazol-1-yl]-3-pyridyl]ethanol;
1-[2-[1-(2-methoxyethyl)-3-methyl-pyrazol-4-yl]-6-[5-[(6-methylpyridazin-3-yl)amino]benzimidazol-1-yl]-3-pyridyl]ethanol;
1-[2-[1-(cyclopropylmethyl)-3-methyl-pyrazol-4-yl]-6-[5-[(6-methylpyridazin-3-yl)amino]benzimidazol-1-yl]-3-pyridyl]ethanol;
5-methyl-1-[3-methylol-6-[6-[(6-methylpyridazin-3-yl)amino]benzimidazol-1-yl]-2-pyridyl]pyrazole-3-carbonitrile;
5-methyl-1-[3-methylol-6-[5-[(6-methylpyridazin-3-yl)amino]benzimidazol-1-yl]-2-pyridyl]pyrazole-3-carbonitrile;
1-[2-(1-ethyl-5-methyl-pyrazol-4-yl)-6-[5-[(6-methylpyridazin-3-yl)amino]benzimidazol-1-yl]-3-pyridyl]ethanol;
1-[2-[1-(cyclopropylmethyl)-5-methyl-pyrazol-4-yl]-6-[5-[(6-methylpyridazin-3-yl)amino]benzimidazol-1-yl]-3-pyridyl]ethanol;
1-[2-[1-(cyclobutylmethyl)-3-methyl-pyrazol-4-yl]-6-[5-[(6-methylpyridazin-3-yl)amino]benzimidazol-1-yl]-3-pyridyl]ethanol;
1-[2-[1-(cyclobutylmethyl)-5-methyl-pyrazol-4-yl]-6-[5-[(6-methylpyridazin-3-yl)amino]benzimidazol-1-yl]-3-pyridyl]ethanol;
1-[3-(1-hydroxyethyl)-6-[5-[(6-methylpyridazin-3-yl)amino]-6-(trifluoromethyl)benzimidazol-1-yl]-2-pyridyl]-5-methyl-pyrazole-3-carbonitrile;
1-[3-[(1S)-1-hydroxyethyl]-6-[5-[(6-methylpyridazin-3-yl)amino]benzimidazol-1-yl]-2-pyridyl]-5-methyl-pyrazole-3-carboximidic acid methyl ester;
1-[2-[1-(2,2-difluorocyclopropyl)-3-methyl-pyrazol-4-yl]-6-[5-[(6-methylpyridazin-3-yl)amino]benzimidazol-1-yl]-3-pyridyl]ethanol;
1-[2-[1-(2-methoxypropyl)-3-methyl-pyrazol-4-yl]-6-[5-[(6-methylpyridazin-3-yl)amino]benzimidazol-1-yl]-3-pyridyl]ethanol;
1-[2-[1-(2-methoxypropyl)-5-methyl-pyrazol-4-yl]-6-[5-[(6-methylpyridazin-3-yl)amino]benzimidazol-1-yl]-3-pyridyl]ethanol;
1-[3-[(1S)-1-hydroxyethyl]-6-[6-[(6-methylpyridazin-3-yl)amino]benzimidazol-1-yl]-2-pyridyl]-5-methyl-pyrazole-3-carbonitrile;
1-[2-[1-(2,2-difluoropropyl)-3-methyl-pyrazol-4-yl]-6-[5-[(6-methylpyridazin-3-yl)amino]benzimidazol-1-yl]-3-pyridyl]ethanol;
1-[3-(1-hydroxyethyl)-6-[5-[(6-methylpyridazin-3-yl)amino]-6-(oxetan-3-yloxy)benzimidazol-1-yl]-2-pyridyl]-5-methyl-pyrazole-3-carbonitrile;
1-[2-(2-ethyl-5-methyl-4-pyridyl)-6-[5-[(6-methylpyridazin-3-yl)amino]benzimidazol-1-yl]-3-pyridyl]ethanol;
1-[2-[1-(2,2-difluoropropyl)-5-methyl-pyrazol-4-yl]-6-[5-[(6-methylpyridazin-3-yl)amino]benzimidazol-1-yl]-3-pyridyl]ethanol;
1-[2-(2-methoxy-5-methyl-4-pyridyl)-6-[5-[(6-methylpyridazin-3-yl)amino]benzimidazol-1-yl]-3-pyridyl]ethanol; (1S)-1-[2-[3,5-bis(difluoromethyl)pyrazol-1-yl]-6-[5-[(6-methylpyridazin-3-yl)amino]benzimidazol-1-yl]-3-pyridyl]ethanol; (1R)-1-[2-[3,5-bis(difluoromethyl)pyrazol-1-yl]-6-[5-[(6-methylpyridazin-3-yl)amino]benzimidazol-1-yl]-3-pyridyl]ethanol;
1-[2-(2-cyclopropyl-5-methyl-4-pyridyl)-6-[5-[(6-methylpyridazin-3-yl)amino]benzimidazol-1-yl]-3-pyridyl]ethanol;
1-[2-[3-(difluoromethoxy)-5-methyl-pyrazol-1-yl]-6-[5-[(6-methylpyridazin-3-yl)amino]benzimidazol-1-yl]-3-pyridyl]ethanol;
1-[3-(1-hydroxyethyl)-6-[6-[(6-methylpyridazin-3-yl)amino]benzimidazol-1-yl]-2-pyridyl]-5-methyl-6,7-dihydro-4˜{H}-pyrazolo[4,3-c]pyridine-3-carbonitrile;
1-[3-(1-hydroxyethyl)-6-[5-[(6-methyl-4-methylsulfonyl-pyridazin-3-yl)amino]benzimidazol-1-yl]-2-pyridyl]-5-methyl-pyrazole-3-carbonitrile;
1-[3-(1-hydroxyethyl)-6-[5-[[6-[2-[2-[2-[2-[2-[2-(2-methoxyethoxy)ethoxy]ethoxy]ethoxy]ethoxy]ethoxy]ethoxy]pyridazin-3-yl]amino]benzimidazol-1-yl]-2-pyridyl]-5-methyl-pyrazole-3-carbonitrile;
1-[3-(1-hydroxyethyl)-6-[6-[(6-methyl-3-pyridyl)amino]-5-(2-morpholinoethoxy)benzimidazol-1-yl]-2-pyridyl]-5-methyl-pyrazole-3-carbonitrile;
1-[3-(1-hydroxyethyl)-6-[6-(2-oxa-5-azaspiro[3.4]octan-5-yl)benzimidazol-1-yl]-2-pyridyl]-5-methyl-pyrazole-3-carbonitrile;
1-[3-(1-hydroxyethyl)-6-[5-(2-oxa-5-azaspiro[3.4]octan-5-yl)benzimidazol-1-yl]-2-pyridyl]-5-methyl-pyrazole-3-carbonitrile;
1-[3-(1-hydroxyethyl)-6-[5-[(6-methyl-3-pyridyl)amino]-6-(2-morpholinoethoxy)benzimidazol-1-yl]-2-pyridyl]-5-methyl-pyrazole-3-carbonitrile;
1-[2-[3-(difluoromethyl)pyrrolidin-1-yl]-6-[5-[(6-methylpyridazin-3-yl)amino]benzimidazol-1-yl]-3-pyridyl]ethanol;
1-[2-(2,2-difluoro-5-azaspiro[2.4]heptan-5-yl)-6-[5-[(6-methylpyridazin-3-yl)amino]benzimidazol-1-yl]-3-pyridyl]ethanol; (1S)-1-[2-[3-(difluoromethyl)-5-methyl-pyrazol-1-yl]-6-[6-fluoro-5-[(6-methylpyridazin-3-yl)amino]benzimidazol-1-yl]-3-pyridyl]ethanol;
1-[6-[6-fluoro-5-[(6-methylpyridazin-3-yl)amino]benzimidazol-1-yl]-2-[3-methyl-1-(2,2,2-trifluoroethyl)pyrazol-4-yl]-3-pyridyl]ethanol; 5-(difluoromethyl)-2-[3-(1-hydroxyethyl)-6-[5-[(6-methylpyridazin-3-yl)amino]benzimidazol-1-yl]-2-pyridyl]pyrazole-3-carbonitrile;
1-[3-(1-hydroxyethyl)-6-[5-[[6-[1-(oxetan-3-yl)pyrrolidin-2-yl]pyridazin-3-yl]amino]benzimidazol-1-yl]-2-pyridyl]-5-methyl-pyrazole-3-carbonitrile; 7,7-difluoro-1-[3-(1-hydroxyethyl)-6-[5-[(6-methylpyridazin-3-yl)amino]benzimidazol-1-yl]-2-pyridyl]-5,6-dihydro-4˜{H}-pyrazolo[4,3-c]pyridine-3-carbonitrile; 5-(difluoromethyl)-1-[3-(1-hydroxyethyl)-6-[5-[(6-methylpyridazin-3-yl)amino]benzimidazol-1-yl]-2-pyridyl]pyrazole-3-carbonitrile; (1S)-1-[2-[3-(difluoromethyl)-5-methyl-pyrazol-1-yl]-6-[6-fluoro-5-(pyridazin-3-ylamino)benzimidazol-1-yl]-3-pyridyl]ethanol;
1-[6-[5-[(6-methylpyridazin-3-yl)amino]benzimidazol-1-yl]-2-[5-methyl-2-(2,2,2-trifluoroethyl)triazol-4-yl]-3-pyridyl]ethanol;
1-[3-(1-hydroxyethyl)-6-[5-[(6-methylpyridazin-3-yl)amino]benzimidazol-1-yl]-2-pyridyl]-4,5-dimethyl-pyrazole-3-carbonitrile;
1-[3-(1-hydroxyethyl)-6-[5-[(4-methyl-2,3-dihydropyridazino[4,5-b][1,4]oxazin-8-yl)amino]benzimidazol-1-yl]-2-pyridyl]-5-methyl-pyrazole-3-carbonitrile;
1-[6-[5-[(6-methylpyridazin-3-yl)amino]benzimidazol-1-yl]-2-[5-methyl-1-(2,2,2-trifluoroethyl)triazol-4-yl]-3-pyridyl]ethanol;
1-[1-[6-(3-cyano-5-methyl-pyrazol-1-yl)-5-(1-hydroxyethyl)-2-pyridyl]benzimidazol-5-yl]-N,N-dimethyl-pyrrolidine-2-carboxamide;
1-[2-[1-(2,2-difluoroethyl)-3-methyl-pyrazol-4-yl]-6-[5-[(6-methylpyridazin-3-yl)amino]benzimidazol-1-yl]-3-pyridyl]ethanol;
1-[6-[5-fluoro-6-[(6-methylpyridazin-3-yl)amino]benzimidazol-1-yl]-3-(hydroxymethyl)-2-pyridyl]-5-methyl-pyrazole-3-carbonitrile; [6-[5-[(6-methylpyridazin-3-yl)amino]benzimidazol-1-yl]-2-[3-methyl-1-(2,2,2-trifluoroethyl)pyrazol-4-yl]-3-pyridyl]methanol; [6-[5-[(6-methylpyridazin-3-yl)amino]benzimidazol-1-yl]-2-[5-methyl-1-(2,2,2-trifluoroethyl)pyrazol-4-yl]-3-pyridyl]methanol; [2-[3-(difluoromethyl)-5-methyl-pyrazol-1-yl]-6-[5-(7,8-dihydro-5H-pyrano[4,3-c]pyridazin-3-ylamino)benzimidazol-1-yl]-3-pyridyl]methanol; [6-[5-[(4aS,7aR)-4-methyl-2,3,4a,5,7,7a-hexahydropyrrolo[3,4-b][1,4]oxazin-6-yl]benzimidazol-1-yl]-2-[3-(difluoromethyl)-5-methyl-pyrazol-1-yl]-3-pyridyl]methanol; [2-[3-(difluoromethyl)-5-methyl-pyrazol-1-yl]-6-[6-fluoro-5-[(6-methylpyridazin-3-yl)amino]benzimidazol-1-yl]-3-pyridyl]methanol;
1-[6-[5-[(6-methylpyridazin-3-yl)amino]benzimidazol-1-yl]-2-[2-(trifluoromethyl)morpholin-4-yl]-3-pyridyl]ethanol;
3-[3-(1-hydroxyethyl)-6-[5-[(6-methylpyridazin-3-yl)amino]benzimidazol-1-yl]-2-pyridyl]-3-azabicyclo[3.1.0]hexane-1-carbonitrile;
1-[2-(5,5-difluoro-2-azabicyclo[2.2.1]heptan-2-yl)-6-[5-[(6-methylpyridazin-3-yl)amino]benzimidazol-1-yl]-3-pyridyl]ethanol;
1-[2-[2-(difluoromethyl)morpholin-4-yl]-6-[5-[(6-methylpyridazin-3-yl)amino]benzimidazol-1-yl]-3-pyridyl]ethanol;
1-[2-(6,6-difluoro-2-azabicyclo[2.2.1]heptan-2-yl)-6-[5-[(6-methylpyridazin-3-yl)amino]benzimidazol-1-yl]-3-pyridyl]ethanol;
1-[2-[7-(difluoromethyl)-5-azaspiro[2.4]heptan-5-yl]-6-[5-[(6-methylpyridazin-3-yl)amino]benzimidazol-1-yl]-3-pyridyl]ethanol;
1-[2-(6,6-difluoro-3-azabicyclo[3.2.0]heptan-3-yl)-6-[5-[(6-methylpyridazin-3-yl)amino]benzimidazol-1-yl]-3-pyridyl]ethanol;
N-[1-[6-(3-cyano-5-methyl-pyrazol-1-yl)-5-(1-hydroxyethyl)-2-pyridyl]benzimidazol-5-yl]cyclopropanecarboxamide;
N-[3-[6-(3-cyano-5-methyl-pyrazol-1-yl)-5-(1-hydroxyethyl)-2-pyridyl]benzimidazol-5-yl]cyclopropanecarboxamide;
1-[2-(4,7-diazaspiro[2.5]octan-7-yl)-6-[5-[(6-methylpyridazin-3-yl)amino]benzimidazol-1-yl]-3-pyridyl]ethanol;
1-[6-[5-[(3aS,6aS)-6-oxo-2,3,3a,4,5,6a-hexahydropyrrolo[2,3-c]pyrrol-1-yl]benzimidazol-1-yl]-3-(1-hydroxyethyl)-2-pyridyl]-5-methyl-pyrazole-3-carbonitrile;
1-[6-[5-[(6-methylpyridazin-3-yl)amino]benzimidazol-1-yl]-2-[3-(trifluoromethyl)piperazin-1-yl]-3-pyridyl]ethanol;
1-[6-[5-[(6-methylpyridazin-3-yl)amino]benzimidazol-1-yl]-2-[5-methyl-2-(trifluoromethyl)-4-pyridyl]-3-pyridyl]ethanol;
1-[3-(1-hydroxyethyl)-6-[5-[(2-keto-1-methyl-3-pyridyl)amino]benzimidazol-1-yl]-2-pyridyl]-5-methyl-pyrazole-3-carbonitrile;
1-[3-(1-hydroxyethyl)-6-[5-(3-methoxy-1-methyl-pyrazol-4-yl)benzimidazol-1-yl]-2-pyridyl]-5-methyl-pyrazole-3-carbonitrile;
1-[2-[2-(difluoromethyl)-5-methyl-4-pyridyl]-6-[5-[(6-methylpyridazin-3-yl)amino]benzimidazol-1-yl]-3-pyridyl]ethanol;
1-[3-(1-hydroxyethyl)-6-[6-(1-methyl-2-oxo-pyrimidin-4-yl)benzimidazol-1-yl]-2-pyridyl]-5-methyl-pyrazole-3-carbonitrile;
1-[2-[3-(difluoromethyl)-5-methyl-pyrazol-1-yl]-6-[6-fluoro-5-[[6-(2-methyl-1,3-dioxolan-2-yl)pyridazin-3-yl]amino]benzimidazol-1-yl]-3-pyridyl]ethanol;
1-[3-(1-hydroxyethyl)-6-[5-(1-methyl-2-oxo-pyrimidin-4-yl)benzimidazol-1-yl]-2-pyridyl]-5-methyl-pyrazole-3-carbonitrile;
1-[3-(1-hydroxyethyl)-6-[6-(6-methylpyridazin-3-yl)benzimidazol-1-yl]-2-pyridyl]-5-methyl-pyrazole-3-carbonitrile;
1-[3-(1-hydroxyethyl)-6-[5-(6-methylpyridazin-3-yl)benzimidazol-1-yl]-2-pyridyl]-5-methyl-pyrazole-3-carbonitrile;
1-[6-[6-[(2S)-2-cyanopyrrolidin-1-yl]benzimidazol-1-yl]-3-(1-hydroxyethyl)-2-pyridyl]-5-methyl-pyrazole-3-carbonitrile;
1-[6-[5-[(2S)-2-cyanopyrrolidin-1-yl]benzimidazol-1-yl]-3-(1-hydroxyethyl)-2-pyridyl]-5-methyl-pyrazole-3-carbonitrile;
1-[6-[6-[(3aS,6aS)-6-oxo-2,3,3a,4,5,6a-hexahydropyrrolo[2,3-c]pyrrol-1-yl]benzimidazol-1-yl]-3-(1-hydroxyethyl)-2-pyridyl]-5-methyl-pyrazole-3-carbonitrile;
1-[2-[3-(difluoromethyl)-5-methyl-1,2,4-triazol-1-yl]-6-[5-[(6-methylpyridazin-3-yl)amino]benzimidazol-1-yl]-3-pyridyl]ethanol;
3-[[1-[6-[3-(difluoromethyl)-5-methyl-pyrazol-1-yl]-5-[(1˜{S})-1-hydroxyethyl]-2-pyridyl]-6-fluoro-benzimidazol-5-yl]amino]-˜{N},˜{N},6-trimethyl-pyridazine-4-carboxamide;
1-[2-[2-(difluoromethoxy)-5-methyl-4-pyridyl]-6-[6-fluoro-5-[(6-methylpyridazin-3-yl)amino]benzimidazol-1-yl]-3-pyridyl]ethanol;
1-[6-[6-fluoro-5-[(6-methylpyridazin-3-yl)amino]benzimidazol-1-yl]-3-(hydroxymethyl)-2-pyridyl]-5-methyl-pyrazole-3-carbonitrile;
3-[[1-[6-[3,5-bis(difluoromethyl)pyrazol-1-yl]-5-(hydroxymethyl)-2-pyridyl]benzimidazol-5-yl]amino]-N,N,6-trimethyl-pyridazine-4-carboxamide;
1-[6-[5-[(6-methylpyridazin-3-yl)amino]benzimidazol-1-yl]-2-[4-methyl-1-(2,2,2-trifluoroethyl)pyrazol-3-yl]-3-pyridyl]ethanol;
1-[2-[3-methyl-1-(2,2,2-trifluoroethyl)pyrazol-4-yl]-6-[5-[6-(morpholinomethyl)pyridazin-3-yl]oxybenzimidazol-1-yl]-3-pyridyl]ethanol;
1-[6-[5-(6-methylpyridazin-3-yl)oxybenzimidazol-1-yl]-2-[3-methyl-(2,2,2-trifluoroethyl)pyrazol-4-yl]-3-pyridyl]ethanol;
1-[6-[5-[6-[(4-methylpiperazin-1-yl)methyl]pyridazin-3-yl]oxybenzimidazol-1-yl]-2-[3-methyl-1-(2,2,2-trifluoroethyl)pyrazol-4-yl]-3-pyridyl]ethanol;
1-[2-[3-(difluoromethoxy)-5-methyl-pyrazol-1-yl]-6-[6-[(6-methylpyridazin-3-yl)amino]-5-(oxetan-3-yloxy)benzimidazol-1-yl]-3-pyridyl]ethanol;
1-[2-(3-chloro-5-methyl-pyrazol-1-yl)-6-[5-fluoro-6-[(6-methylpyridazin-3-yl)amino]benzimidazol-1-yl]-3-pyridyl]ethanol;
1-[2-(3-chloro-5-methyl-pyrazol-1-yl)-6-[6-fluoro-5-[(6-methylpyridazin-3-yl)amino]benzimidazol-1-yl]-3-pyridyl]ethanol;
1-[2-[i-(cyclopropylmethyl)-3-methyl-pyrazol-4-yl]-6-[6-fluoro-5-[(6-methylpyridazin-3-yl)amino]benzimidazol-1-yl]-3-pyridyl]ethanol;
1-[2-[3-(difluoromethoxy)-5-methyl-pyrazol-1-yl]-6-[5-[(6-methylpyridazin-3-yl)amino]-6-(oxetan-3-yloxy)benzimidazol-1-yl]-3-pyridyl]ethanol;
N-[1-[5-(1-hydroxyethyl)-6-[3-methyl-(2,2,2-trifluoroethyl)pyrazol-4-yl]-2-pyridyl]benzimidazol-5-yl]-2-(1-piperidyl)acetamide; 6-[3-(1-hydroxyethyl)-6-[5-[(6-methylpyridazin-3-yl)amino]benzimidazol-1-yl]-2-pyridyl]-6-azaspiro[3.4]octane-8-carbonitrile; 4-[3-(1-hydroxyethyl)-6-[5-[(6-methylpyridazin-3-yl)amino]benzimidazol-1-yl]-2-pyridyl]morpholine-2-carbonitrile;
N-[1-[6-[3-(difluoromethyl)-5-methyl-pyrazol-1-yl]-5-(1-hydroxyethyl)-2-pyridyl]-6-fluoro-benzimidazol-5-yl]cyclopropanecarboxamide;
1-[6-[6-(2,3-difluoro-4-methyl-anilino)-5-fluoro-benzimidazol-1-yl]-2-[3-(difluoromethyl)-5-methyl-pyrazol-1-yl]-3-pyridyl]ethanol;
1-[2-[1-(difluoromethyl)-4-methyl-pyrazol-3-yl]-6-[5-[(6-methylpyridazin-3-yl)amino]benzimidazol-1-yl]-3-pyridyl]ethanol;
1-[2-[3-(difluoromethyl)-5-methyl-pyrazol-1-yl]-6-[5-fluoro-6-(2,3,4-trifluoroanilino)benzimidazol-1-yl]-3-pyridyl]ethanol;
1-[3-(1-hydroxyethyl)-6-[6-(6-methyl-7-oxo-5H-pyrrolo[3,4-b]pyridin-2-yl)benzimidazol-1-yl]-2-pyridyl]-5-methyl-pyrazole-3-carbonitrile;
1-[3-(1-hydroxyethyl)-6-[5-(6-methyl-7-oxo-5H-pyrrolo[3,4-b]pyridin-2-yl)benzimidazol-1-yl]-2-pyridyl]-5-methyl-pyrazole-3-carbonitrile;
1-[2-[3-(difluoromethoxy)-5-methylpyrazol-1-yl]-6-[5-[[6-(difluoromethyl)pyridazin-3-yl]amino]benzimidazol-1-yl]pyridin-3-yl]ethanol;
1-[2-[3-(difluoromethyl)-5-ethyl-pyrazol-1-yl]-6-[6-[(6-methylpyridazin-3-yl)amino]benzimidazol-1-yl]-3-pyridyl]ethanol;
1-[2-[3-(difluoromethyl)-4,5-dimethyl-pyrazol-1-yl]-6-[5-[(6-methylpyridazin-3-yl)amino]benzimidazol-1-yl]-3-pyridyl]ethanol;
1-[2-[3-(difluoromethyl)-5-ethyl-pyrazol-1-yl]-6-[5-[(6-methylpyridazin-3-yl)amino]benzimidazol-1-yl]-3-pyridyl]ethanol;
1-[6-[6-[(6-chloropyridazin-3-yl)amino]-5-fluoro-benzimidazol-1-yl]-2-[3-(difluoromethyl)-5-methyl-pyrazol-1-yl]-3-pyridyl]ethanol;
1-[6-[5-[(6-chloropyridazin-3-yl)amino]-6-fluoro-benzimidazol-1-yl]-2-[3-(difluoromethyl)-5-methyl-pyrazol-1-yl]-3-pyridyl]ethanol;
(1S)-1-[2-[3-(difluoromethoxy)-5-methyl-pyrazol-1-yl]-6-[6-fluoro-5-(pyridazin-3-ylamino)benzimidazol-1-yl]-3-pyridyl]ethanol;
(1S)-1-[2-[3-(difluoromethoxy)-5-methyl-pyrazol-1-yl]-6-[6-fluoro-5-[(6-methylpyridazin-3-yl)amino]benzimidazol-1-yl]-3-pyridyl]ethanol;
(1R)-1-[2-[3-(difluoromethoxy)-5-methyl-pyrazol-1-yl]-6-[6-fluoro-5-[(6-methylpyridazin-3-yl)amino]benzimidazol-1-yl]-3-pyridyl]ethanol;
1-[6-[5-fluoro-6-[(5-methyl-1,3,4-oxadiazol-2-yl)amino]benzimidazol-1-yl]-3-(1-hydroxyethyl)-2-pyridyl]-5-methyl-pyrazole-3-carbonitrile;
1-[6-[6-fluoro-5-[(5-methyl-1,3,4-oxadiazol-2-yl)amino]benzimidazol-1-yl]-3-(1-hydroxyethyl)-2-pyridyl]-5-methyl-pyrazole-3-carbonitrile;
1-[6-[6-fluoro-5-(2,3,4-trifluoroanilino)benzimidazol-1-yl]-3-(1-hydroxyethyl)-2-pyridyl]-5-methyl-pyrazole-3-carbonitrile;
1-[6-[5-fluoro-6-(2,3,4-trifluoroanilino)benzimidazol-1-yl]-3-(1-hydroxyethyl)-2-pyridyl]-5-methyl-pyrazole-3-carbonitrile;
N-[1-[6-(3-cyano-5-methyl-pyrazol-1-yl)-5-(1-hydroxyethyl)-2-pyridyl]benzimidazol-5-yl]-1-fluoro-cyclopropanecarboxamide;
1-[6-[6-(6,7-dihydro-5˜{H}-cyclopenta[c]pyridazin-3-ylamino)-5-fluoro-benzimidazol-1-yl]-3-(1-hydroxyethyl)-2-pyridyl]-5-methyl-pyrazole-3-carbonitrile;
1-[6-[5-(6,7-dihydro-5˜{H}-cyclopenta[c]pyridazin-3-ylamino)-6-fluoro-benzimidazol-1-yl]-3-(1-hydroxyethyl)-2-pyridyl]-5-methyl-pyrazole-3-carbonitrile;
(1S)-1-[2-[3-(difluoromethoxy)-5-methyl-pyrazol-1-yl]-6-[5-[(6-methylpyridazin-3-yl)amino]-6-(oxetan-3-yloxy)benzimidazol-1-yl]-3-pyridyl]ethanol;
(1R)-1-[2-[3-(difluoromethoxy)-5-methyl-pyrazol-1-yl]-6-[5-[(6-methylpyridazin-3-yl)amino]-6-(oxetan-3-yloxy)benzimidazol-1-yl]-3-pyridyl]ethanol;
1-[6-[6-fluoro-5-[(5-methyl-1,3,4-thiadiazol-2-yl)amino]benzimidazol-1-yl]-3-(1-hydroxyethyl)-2-pyridyl]-5-methyl-pyrazole-3-carbonitrile;
3-fluoro-4-[3-(1-hydroxyethyl)-6-[5-[(6-methylpyridazin-3-yl)amino]benzimidazol-1-yl]-2-pyridyl]-1-(2,2,2-trifluoroethyl)pyridin-2-one;
1-[2-[3,5-bis(difluoromethyl)pyrazol-1-yl]-6-[6-fluoro-5-[(6-methylpyridazin-3-yl)amino]benzimidazol-1-yl]-3-pyridyl]ethanol;
1-[6-[6-(difluoromethoxy)-5-[(6-methylpyridazin-3-yl)amino]benzimidazol-1-yl]-3-(1-hydroxyethyl)-2-pyridyl]-5-methyl-pyrazole-3-carbonitrile;
1-[6-[6-fluoro-5-[(5-fluoro-6-methyl-3-pyridyl)amino]benzimidazol-1-yl]-3-(1-hydroxyethyl)-2-pyridyl]-5-methyl-pyrazole-3-carbonitrile;
1-[6-[5-[[5-(azetidin-1-yl)pyridazin-3-yl]amino]-6-fluoro-benzimidazol-1-yl]-2-[3-(difluoromethyl)-5-methyl-pyrazol-1-yl]-3-pyridyl]ethanol;
1-[6-[6-fluoro-5-[(5-fluoro-6-methyl-3-pyridyl)amino]benzimidazol-1-yl]-3-[(1S)-1-hydroxyethyl]-2-pyridyl]-5-methyl-pyrazole-3-carbonitrile;
1-[3-(1-hydroxyethyl)-6-[5-[2-(2-oxopyrrolidin-1-yl)oxazol-5-yl]benzimidazol-1-yl]-2-pyridyl]-5-methyl-pyrazole-3-carbonitrile;
1-[2-(4,4-difluoropyrrolidin-3-yl)oxy-6-[5-[(6-methylpyridazin-3-yl)amino]benzimidazol-1-yl]-3-pyridyl]ethanol;
1-[6-[5-(7,8-dihydro-5˜{H}-pyrano[4,3-c]pyridazin-3-ylamino)-6-methoxy-benzimidazol-1-yl]-3-(1-hydroxyethyl)-2-pyridyl]-5-methyl-pyrazole-3-carbonitrile;
1-[3-(1-hydroxyethyl)-6-[6-[2-(2-oxopyrrolidin-1-yl)oxazol-5-yl]benzimidazol-1-yl]-2-pyridyl]-5-methyl-pyrazole-3-carbonitrile;
1-[2-[6-(difluoromethoxy)-3-methyl-pyridazin-4-yl]-6-[5-[(6-methylpyridazin-3-yl)amino]benzimidazol-1-yl]-3-pyridyl]ethanol;
1-[2-[3-(difluoromethyl)-5-methoxy-pyrazol-1-yl]-6-[6-[(6-methylpyridazin-3-yl)amino]benzimidazol-1-yl]-3-pyridyl]ethanol;
1-[2-[3-(difluoromethyl)-5-methoxy-pyrazol-1-yl]-6-[5-[(6-methylpyridazin-3-yl)amino]benzimidazol-1-yl]-3-pyridyl]ethanol;
1-[2-[1-(2,3-difluoropropyl)-3-methyl-pyrazol-4-yl]-6-[5-[(6-methylpyridazin-3-yl)amino]benzimidazol-1-yl]-3-pyridyl]ethanol;
(1S)-1-[2-[3-(difluoromethoxy)-5-methyl-pyrazol-1-yl]-6-[6-methoxy-5-[(2-methylpyrimidin-5-yl)amino]benzimidazol-1-yl]-3-pyridyl]ethanol;
1-[6-[5-[(6-methylpyridazin-3-yl)amino]benzimidazol-1-yl]-2-[2-methyl-5-(2,2,2-trifluoroethyl)-1,2,4-triazol-3-yl]-3-pyridyl]ethanol;
1-[6-[6-(difluoromethoxy)-5-[(6-methylpyridazin-3-yl)amino]benzimidazol-1-yl]-3-[(1S)-1-hydroxyethyl]-2-pyridyl]-5-methyl-pyrazole-3-carbonitrile;
1-[6-[6-(difluoromethoxy)-5-[(6-methylpyridazin-3-yl)amino]benzimidazol-1-yl]-3-[(1R)-1-hydroxyethyl]-2-pyridyl]-5-methyl-pyrazole-3-carbonitrile;
1-[2-[3-(difluoromethoxy)-5-(difluoromethyl)pyrazol-1-yl]-6-[5-[(6-methylpyridazin-3-yl)amino]benzimidazol-1-yl]-3-pyridyl]ethanol;
1-[2-[3-(difluoromethoxy)-5-(difluoromethyl)pyrazol-1-yl]-6-[6-[(6-methylpyridazin-3-yl)amino]benzimidazol-1-yl]-3-pyridyl]ethanol;
1-[2-[3-(difluoromethoxy)-5-methyl-pyrazol-1-yl]-6-[6-[(6-methylpyridazin-3-yl)amino]-5-(oxetan-3-ylmethyl)benzimidazol-1-yl]-3-pyridyl]ethanol; and
1-[2-[3-(difluoromethoxy)-5-methyl-pyrazol-1-yl]-6-[5-[(6-methylpyridazin-3-yl)amino]-6-(oxetan-3-ylmethyl)benzimidazol-1-yl]-3-pyridyl]ethanol;
   or a pharmaceutically acceptable salt thereof.

67. A compound according to any one of embodiments 1 to 66, selected from
[2-(3-chlorophenyl)-6-(5,6-dimethoxybenzimidazol-1-yl)-3-pyridyl]methanol;
(2-(3-chloro-2-fluorophenyl)-6-(5,6-dimethoxy-1H-benzo[d]imidazol-1-yl)pyridin-3-yl)methanol;
(6-(5,6-dimethoxy-1H-benzo[d]imidazol-1-yl)-2-(4-fluoro-2-methoxyphenyl)pyridin-3-yl)methanol;
1-(6-(5,6-dimethoxy-1H-benzo[d]imidazol-1-yl)-2-(4-fluoro-2-methoxyphenyl)pyridin-3-yl)ethan-1-ol;
1-(6-(5,6-dimethoxy-1H-benzo[d]imidazol-1-yl)-2-(4-fluoro-2-methoxyphenyl)pyridin-3-yl)propan-1-ol;
1-(2-(3-chlorophenyl)-6-(5,6-dimethoxy-1H-benzo[d]imidazol-1-yl)pyridin-3-yl)ethan-1-ol;
1-(2-(3-chloro-2-fluorophenyl)-6-(5,6-dimethoxy-1H-benzo[d]imidazol-1-yl)pyridin-3-yl)ethan-1-ol;
(S)-1-(6-(5,6-dimethoxy-1H-benzo[d]imidazol-1-yl)-2-(4-fluoro-2-methoxyphenyl)pyridin-3-yl)ethan-1-ol;
(R)-1-(6-(5,6-dimethoxy-1H-benzo[d]imidazol-1-yl)-2-(4-fluoro-2-methoxyphenyl)pyridin-3-yl)ethan-1-ol;
cyclopropyl(6-(5,6-dimethoxy-1H-benzo[d]imidazol-1-yl)-2-(4-fluoro-2-methoxyphenyl)pyridin-3-yl)methanol;
1-(2-(3-chlorophenyl)-6-(5,6-dimethoxy-1H-benzo[d]imidazol-1-yl)pyridin-3-yl)propan-1-ol;
(2-(3-chlorophenyl)-6-(5,6-dimethoxy-1H-benzo[d]imidazol-1-yl)pyridin-3-yl)(cyclopropyl)methanol;
(6-(5,6-dimethoxy-1H-benzo[d]imidazol-1-yl)-2-morpholinopyridin-3-yl)methanol;
(6-(5,6-dimethoxy-1H-benzo[d]imidazol-1-yl)-2-(pyrrolidin-1-yl)pyridin-3-yl)methanol;
(6-(5,6-dimethoxy-1H-benzo[d]imidazol-1-yl)-2-(piperidin-1-yl)pyridin-3-yl)methanol;
5-((6-(5,6-dimethoxy-1H-benzo[d]imidazol-1-yl)-3-(hydroxymethyl)pyridin-2-yl)amino)pentan-1-ol;
1-(6-(5,6-dimethoxy-1H-benzo[d]imidazol-1-yl)-3-(hydroxymethyl)pyridin-2-yl)pyrrolidin-2-one;
4-((6-(5,6-dimethoxy-1H-benzo[d]imidazol-1-yl)-3-(hydroxymethyl)pyridin-2-yl)amino)butan-1-ol;
(S)-2-(6-(5,6-dimethoxy-1H-benzo[d]imidazol-1-yl)-3-(1-hydroxyethyl)pyridin-2-yl)benzamide;
(R)-2-(6-(5,6-dimethoxy-1H-benzo[d]imidazol-1-yl)-3-(1-hydroxyethyl)pyridin-2-yl)benzamide;
(S)-3-(6-(5,6-dimethoxy-1H-benzo[d]imidazol-1-yl)-3-(1-hydroxyethyl)pyridin-2-yl)benzonitrile;
(R)-3-(6-(5,6-dimethoxy-1H-benzo[d]imidazol-1-yl)-3-(1-hydroxyethyl)pyridin-2-yl)benzonitrile;
(S)-4-(6-(5,6-dimethoxy-1H-benzo[d]imidazol-1-yl)-3-(1-hydroxyethyl)pyridin-2-yl)benzonitrile;
(R)-4-(6-(5,6-dimethoxy-1H-benzo[d]imidazol-1-yl)-3-(1-hydroxyethyl)pyridin-2-yl)benzonitrile;
(S)-6-(5,6-dimethoxy-1H-benzo[d]imidazol-1-yl)-3-(1-hydroxyethyl)-1′-methyl-[2,4′-bipyridin]-2′(1′H)-one;
(R)-6-(5,6-dimethoxy-1H-benzo[d]imidazol-1-yl)-3-(1-hydroxyethyl)-1′-methyl-[2,4′-bipyridin]-2′(1′H)-one;
1-[6-[5-(2-morpholinoethoxy)benzimidazol-1-yl]-2-phenoxy-3-pyridyl]ethanol;
1-[2-anilino-6-[5-(2-morpholinoethoxy)benzimidazol-1-yl]-3-pyridyl]ethanol;
1-[6-[5-(2-morpholinoethoxy)benzimidazol-1-yl]-2-[3-(trifluoromethyl)pyrazol-1-yl]-3-pyridyl]ethanol;
1-[6-(5,6-dimethoxybenzimidazol-1-yl)-2-[3-(trifluoromethyl)pyrazol-1-yl]-3-pyridyl]ethanol;
1-[3-(1-hydroxyethyl)-6-[5-[(6-methylpyridazin-3-yl)amino]benzimidazol-1-yl]-2-pyridyl]-5-methyl-pyrazole-3-carbonitrile;
1-[3-(1-hydroxyethyl)-6-[5-(4-methylpiperazin-1-yl)benzimidazol-1-yl]-2-pyridyl]-5-methyl-pyrazole-3-carbonitrile;
1-[3-(1-hydroxyethyl)-6-[5-[4-(oxetan-3-yl)piperazin-1-yl]benzimidazol-1-yl]-2-pyridyl]-5-methyl-pyrazole-3-carbonitrile;
1-[3-(1-hydroxyethyl)-6-[5-(2-morpholinoethoxy)benzimidazol-1-yl]-2-pyridyl]-5-methyl-pyrazole-3-carbonitrile;
1-[3-(1-hydroxyethyl)-6-[5-[[1-(oxetan-3-yl)pyrazol-4-yl]amino]benzimidazol-1-yl]-2-pyridyl]-5-methyl-pyrazole-3-carbonitrile;
1-[6-[5-[[1-(azetidin-3-yl)pyrazol-4-yl]amino]benzimidazol-1-yl]-3-(1-hydroxyethyl)-2-pyridyl]-5-methyl-pyrazole-3-carbonitrile;
1-[3-(1-hydroxyethyl)-6-[6-(6-methylpyridazin-3-yl)oxybenzimidazol-1-yl]-2-pyridyl]-5-methyl-pyrazole-3-carbonitrile;
1-[3-(1-hydroxyethyl)-6-[5-(6-methylpyridazin-3-yl)oxybenzimidazol-1-yl]-2-pyridyl]-5-methyl-pyrazole-3-carbonitrile;
5-[3-(1-hydroxyethyl)-6-[5-[(6-methylpyridazin-3-yl)amino]benzimidazol-1-yl]-2-pyridyl]-1-methyl-pyrazole-4-carbonitrile;
1-[3-(1-hydroxyethyl)-6-[5-[rac-(3R)-3-hydroxypyrrolidin-1-yl]benzimidazol-1-yl]-2-pyridyl]-5-methyl-pyrazole-3-carbonitrile;
1-[3-(1-hydroxyethyl)-6-[5-[(3S)-3-hydroxypyrrolidin-1-yl]benzimidazol-1-yl]-2-pyridyl]-5-methyl-pyrazole-3-carbonitrile;
1-[3-(1-hydroxyethyl)-6-[5-[(6-methyl-3-pyridyl)amino]benzimidazol-1-yl]-2-pyridyl]-5-methyl-pyrazole-3-carbonitrile;
1-[3-(1-hydroxyethyl)-6-[5-[(5-methylpyrazin-2-yl)amino]benzimidazol-1-yl]-2-pyridyl]-5-methyl-pyrazole-3-carbonitrile;
1-[3-(1-hydroxyethyl)-6-[5-[(5-methylpyrimidin-2-yl)amino]benzimidazol-1-yl]-2-pyridyl]-5-methyl-pyrazole-3-carbonitrile;
1-[3-(1-hydroxyethyl)-6-[5-[(5-methyl-2-pyridyl)amino]benzimidazol-1-yl]-2-pyridyl]-5-methyl-pyrazole-3-carbonitrile;
1-[2-(1,1-dioxo-1,2-thiazolidin-2-yl)-6-[5-[(6-methylpyridazin-3-yl)amino]benzimidazol-1-yl]-3-pyridyl]ethanol;
1-[3-(1-hydroxyethyl)-6-[5-[(2-methylpyrimidin-5-yl)amino]benzimidazol-1-yl]-2-pyridyl]-5-methyl-pyrazole-3-carbonitrile;
1-[3-(1-hydroxyethyl)-6-[5-(pyridazin-4-ylamino)benzimidazol-1-yl]-2-pyridyl]-5-methyl-pyrazole-3-carbonitrile;
1-[3-(1-hydroxyethyl)-6-[5-[(6-methoxypyridazin-3-yl)amino]benzimidazol-1-yl]-2-pyridyl]-5-methyl-pyrazole-3-carbonitrile;
1-[3-(1-hydroxyethyl)-6-[5-(pyridazin-3-ylamino)benzimidazol-1-yl]-2-pyridyl]-5-methyl-pyrazole-3-carbonitrile;
1-[6-[5-[[6-(difluoromethyl)pyridazin-3-yl]amino]benzimidazol-1-yl]-3-(1-hydroxyethyl)-2-pyridyl]-5-methyl-pyrazole-3-carbonitrile;
1-[6-[6-fluoro-5-[(6-methylpyridazin-3-yl)amino]benzimidazol-1-yl]-3-(1-hydroxyethyl)-2-pyridyl]-5-methyl-pyrazole-3-carbonitrile;
1-[6-[5-fluoro-6-[(6-methylpyridazin-3-yl)amino]benzimidazol-1-yl]-3-(1-hydroxyethyl)-2-pyridyl]-5-methyl-pyrazole-3-carbonitrile;
1-[6-[5-[(6-methylpyridazin-3-yl)amino]benzimidazol-1-yl]-2-[3-(trifluoromethyl)-4,5,6,7-tetrahydropyrazolo[4,3-c]pyridin-1-yl]-3-pyridyl]ethanol;
1-[6-[6-[(6-methylpyridazin-3-yl)amino]benzimidazol-1-yl]-2-[3-(trifluoromethyl)-4,5,6,7-tetrahydropyrazolo[4,3-c]pyridin-1-yl]-3-pyridyl]ethanol;
1-[3-(1-hydroxyethyl)-6-[5-[(5-methyl-1,3,4-oxadiazol-2-yl)amino]benzimidazol-1-yl]-2-pyridyl]-5-methyl-pyrazole-3-carbonitrile;
2-[3-(1-hydroxyethyl)-6-[5-[(6-methylpyridazin-3-yl)amino]benzimidazol-1-yl]-2-pyridyl]-5-methyl-pyrazole-3-carbonitrile;
(3R)-1-[3-(1-hydroxyethyl)-6-[6-[(6-methylpyridazin-3-yl)amino]benzimidazol-1-yl]-2-pyridyl]pyrrolidine-3-carbonitrile;
rac-(3R)-1-[3-(1-hydroxyethyl)-6-[5-[(6-methylpyridazin-3-yl)amino]benzimidazol-1-yl]-2-pyridyl]pyrrolidine-3-carbonitrile;
1-[2-(3,5-dimethylpyrazol-1-yl)-6-[6-[(6-methylpyridazin-3-yl)amino]benzimidazol-1-yl]-3-pyridyl]ethanol;
1-[2-(3,5-dimethylpyrazol-1-yl)-6-[5-[(6-methylpyridazin-3-yl)amino]benzimidazol-1-yl]-3-pyridyl]ethanol;
5-methyl-1-[6-[5-[(6-methylpyridazin-3-yl)amino]benzimidazol-1-yl]-3-[rac-(1R)-1-hydroxyethyl]-2-pyridyl]pyrazole-3-carbonitrile;
5-methyl-1-[6-[5-[(6-methylpyridazin-3-yl)amino]benzimidazol-1-yl]-3-[rac-(1S)-1-hydroxyethyl]-2-pyridyl]pyrazole-3-carbonitrile;
1-[3-(1-hydroxyethyl)-6-[5-[(5-methyl-1,3,4-thiadiazol-2-yl)amino]benzimidazol-1-yl]-2-pyridyl]-5-methyl-pyrazole-3-carbonitrile;
1-[3-(1-hydroxyethyl)-6-[5-[[1-(oxetan-3-yl)-4-piperidyl]oxy]benzimidazol-1-yl]-2-pyridyl]-5-methyl-pyrazole-3-carbonitrile;
1-[3-(1-hydroxyethyl)-6-[6-[[1-(oxetan-3-yl)-4-piperidyl]oxy]benzimidazol-1-yl]-2-pyridyl]-5-methyl-pyrazole-3-carbonitrile;
1-[3-(1-hydroxyethyl)-6-[5-[(6-methylpyridazin-3-yl)amino]benzimidazol-1-yl]-2-pyridyl]pyrazole-3-carbonitrile;
1-[[[3-(1-hydroxyethyl)-6-[5-[(6-methylpyridazin-3-yl)amino]benzimidazol-1-yl]-2-pyridyl]amino]methyl]cyclopropanecarbonitrile;
1-[[[3-(1-hydroxyethyl)-6-[6-[(6-methylpyridazin-3-yl)amino]benzimidazol-1-yl]-2-pyridyl]amino]methyl]cyclopropanecarbonitrile;
5-[3-(1-hydroxyethyl)-6-[5-[(6-methylpyridazin-3-yl)amino]benzimidazol-1-yl]-2-pyridyl]pyridine-3-carbonitrile;
1-[3-(1-hydroxyethyl)-6-[5-(1,2,4-triazin-3-ylamino)benzimidazol-1-yl]-2-pyridyl]-5-methyl-pyrazole-3-carbonitrile;
1-[3-(1-hydroxyethyl)-6-[6-[(6-methylpyridazin-3-yl)amino]benzimidazol-1-yl]-2-pyridyl]-3-methyl-pyrrolidine-3-carbonitrile;
1-[3-(1-hydroxyethyl)-6-[5-[(6-methylpyridazin-3-yl)amino]benzimidazol-1-yl]-2-pyridyl]-3-methyl-pyrrolidine-3-carbonitrile;
1-[6-[5-[(6-methylpyridazin-3-yl)amino]benzimidazol-1-yl]-2-[2-methyl-5-(trifluoromethyl)pyrazol-3-yl]-3-pyridyl]ethanol;
1-[3-(1-hydroxyethyl)-6-[5-(isoxazol-3-ylamino)benzimidazol-1-yl]-2-pyridyl]-5-methyl-pyrazole-3-carbonitrile;
3-(1-hydroxyethyl)-6-[5-[(6-methylpyridazin-3-yl)amino]benzimidazol-1-yl]-N-(2,2,2-trifluoroethyl)pyridine-2-carboxamide;
1-[6-[6-[(6-methylpyridazin-3-yl)amino]benzimidazol-1-yl]-2-[rac-(3aR,6aS)-2,3,3a,5,6,6a-hexahydro-1H-pyrrolo[3,2-b]pyrrol-4-yl]-3-pyridyl]ethanol;
1-[6-[6-[(6-methylpyridazin-3-yl)amino]benzimidazol-1-yl]-2-[rac-(3aS,6aR)-2,3,3a,5,6,6a-hexahydro-1H-pyrrolo[3,2-b]pyrrol-4-yl]-3-pyridyl]ethanol;
1-[6-[5-[(6-methylpyridazin-3-yl)amino]benzimidazol-1-yl]-2-[rac-(3aR,6aS)-2,3,3a,5,6,6a-hexahydro-1H-pyrrolo[3,2-b]pyrrol-4-yl]-3-pyridyl]ethanol;
1-[2-[3-(difluoromethyl)-5-methyl-pyrazol-1-yl]-6-[6-[(6-methylpyridazin-3-yl)amino]benzimidazol-1-yl]-3-pyridyl]ethanol;
1-[2-[3-(difluoromethyl)-5-methyl-pyrazol-1-yl]-6-[5-[(6-methylpyridazin-3-yl)amino]benzimidazol-1-yl]-3-pyridyl]ethanol;
1-[6-[6-bromo-5-[4-(oxetan-3-yl)piperazin-1-yl]benzimidazol-1-yl]-3-(1-hydroxyethyl)-2-pyridyl]-5-methyl-pyrazole-3-carbonitrile;
1-[2-(3-ethoxy-5-methyl-pyrazol-1-yl)-6-[5-[(6-methylpyridazin-3-yl)amino]benzimidazol-1-yl]-3-pyridyl]ethanol;
1-[2-(3-ethoxy-5-methyl-pyrazol-1-yl)-6-[6-[(6-methylpyridazin-3-yl)amino]benzimidazol-1-yl]-3-pyridyl]ethanol;
1-[2-(6-methylpyridazin-4-yl)-6-[5-[(6-methylpyridazin-3-yl)amino]benzimidazol-1-yl]-3-pyridyl]ethanol;
4-[3-(1-hydroxyethyl)-6-[5-[(6-methylpyridazin-3-yl)amino]benzimidazol-1-yl]-2-pyridyl]-1-(2,2,2-trifluoroethyl)piperazin-2-one;
4-[3-(1-hydroxyethyl)-6-[6-[(6-methylpyridazin-3-yl)amino]benzimidazol-1-yl]-2-pyridyl]-1-(2,2,2-trifluoroethyl)piperazin-2-one;
1-[[3-(1-hydroxyethyl)-6-[6-[(6-methylpyridazin-3-yl)amino]benzimidazol-1-yl]-2-pyridyl]oxymethyl]cyclopropanecarbonitrile;
1-[[3-(1-hydroxyethyl)-6-[5-[(6-methylpyridazin-3-yl)amino]benzimidazol-1-yl]-2-pyridyl]oxymethyl]cyclopropanecarbonitrile;
1-[3-(1-hydroxyethyl)-6-[5-methoxy-6-[(6-methylpyridazin-3-yl)amino]benzimidazol-1-yl]-2-pyridyl]-5-methyl-pyrazole-3-carbonitrile;
1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanol;
1-[3-(1-hydroxyethyl)-6-[6-[(6-methylpyridazin-3-yl)amino]benzimidazol-1-yl]-2-pyridyl]-N,5-dimethyl-pyrazole-3-carboxamide;
1-[2-(5-methyl-3-methylsulfonyl-pyrazol-1-yl)-6-[5-[(6-methylpyridazin-3-yl)amino]benzimidazol-1-yl]-3-pyridyl]ethanol;
1-[3-(1-hydroxyethyl)-6-[5-[(2-keto-1-methyl-pyrimidin-4-yl)amino]benzimidazol-1-yl]-2-pyridyl]-5-methyl-pyrazole-3-carbonitrile;
5-methyl-1-[6-[5-[(6-methylpyridazin-3-yl)amino]benzimidazol-1-yl]-3-(2,2,2-trifluoro-1-hydroxy-ethyl)-2-pyridyl]pyrazole-3-carbonitrile;
1-[2-[1-(difluoromethyl)-3-methyl-pyrazol-4-yl]-6-[5-[(6-methylpyridazin-3-yl)amino]benzimidazol-1-yl]-3-pyridyl]ethanol;
1-[3-(1-hydroxyethyl)-6-[5-[(6-methylpyridazin-3-yl)amino]benzimidazol-1-yl]-2-pyridyl]-5-methyl-pyrazole-4-carbonitrile;
1-[3-(1-hydroxyethyl)-6-[6-[(6-methylpyridazin-3-yl)amino]benzimidazol-1-yl]-2-pyridyl]-5-methyl-pyrazole-4-carbonitrile;
1-[3-(1-hydroxyethyl)-6-[5-[(6-methylpyridazin-3-yl)amino]benzimidazol-1-yl]-2-pyridyl]-N,5-dimethyl-pyrazole-3-carboxamide;
1-[3-(1-hydroxyethyl)-6-[5-[(2-keto-1-methyl-4-piperidyl)amino]benzimidazol-1-yl]-2-pyridyl]-5-methyl-pyrazole-3-carbonitrile;
[2-(3-methoxy-5-methyl-pyrazol-1-yl)-6-[5-[(6-methylpyridazin-3-yl)amino]benzimidazol-1-yl]-3-pyridyl]methanol;
1-[2-(3,5-dimethylisoxazol-4-yl)-6-[5-[(6-methylpyridazin-3-yl)amino]benzimidazol-1-yl]-3-pyridyl]ethanol;
1-[3-(1-hydroxyethyl)-6-[5-[(4-methoxypyridazin-3-yl)amino]benzimidazol-1-yl]-2-pyridyl]-5-methyl-pyrazole-3-carbonitrile;
1-[3-(1-hydroxyethyl)-6-[5-[(5-keto-1-methyl-pyrrolidin-3-yl)amino]benzimidazol-1-yl]-2-pyridyl]-5-methyl-pyrazole-3-carbonitrile;
1-[6-[5-[(6-methylpyridazin-3-yl)amino]benzimidazol-1-yl]-2-(3-methyl-4-pyridyl)-3-pyridyl]ethanol;
1-[2-[4-(cyclopropylamino)pyrimidin-5-yl]-6-[5-[(6-methylpyridazin-3-yl)amino]benzimidazol-1-yl]-3-pyridyl]ethanol;
5-[3-(1-hydroxyethyl)-6-[5-[(6-methylpyridazin-3-yl)amino]benzimidazol-1-yl]-2-pyridyl]-4-(trifluoromethyl)pyridin-2-ol;
3-[3-(1-hydroxyethyl)-6-[5-[(6-methylpyridazin-3-yl)amino]benzimidazol-1-yl]-2-pyridyl]-6,7-dihydro-5H-pyrazolo[1,5-a]pyrazin-4-one;
1-[6-[6-fluoro-5-[[(3S,4R)-4-fluoropyrrolidin-3-yl]amino]benzimidazol-1-yl]-3-(1-hydroxyethyl)-2-pyridyl]-5-methyl-pyrazole-3-carbonitrile;
1-[2-[2-(difluoromethoxy)-5-methyl-4-pyridyl]-6-[5-[(6-methylpyridazin-3-yl)amino]benzimidazol-1-yl]-3-pyridyl]ethanol;
1-[2-(2-fluoro-5-methyl-4-pyridyl)-6-[5-[(6-methylpyridazin-3-yl)amino]benzimidazol-1-yl]-3-pyridyl]ethanol;
1-[3-(1-hydroxyethyl)-6-[6-[(6-methylpyridazin-3-yl)amino]benzimidazol-1-yl]-2-pyridyl]-4-methyl-pyrazole-3-carbonitrile;
1-[3-(1-hydroxyethyl)-6-[5-[(6-methylpyridazin-3-yl)amino]benzimidazol-1-yl]-2-pyridyl]-4-methyl-pyrazole-3-carbonitrile;
1-[6-[5-[(6-methylpyridazin-3-yl)amino]benzimidazol-1-yl]-2-[5-methyl-3-(trifluoromethyl)-6,7-dihydro-4H-pyrazolo[4,3-c]pyridin-1-yl]-3-pyridyl]ethanol;
1-[[[3-(1-hydroxyethyl)-6-[6-[(6-methylpyridazin-3-yl)amino]benzimidazol-1-yl]-2-pyridyl]-methyl-amino]methyl]cyclopropanecarbonitrile;
1-[[[3-(1-hydroxyethyl)-6-[5-[(6-methylpyridazin-3-yl)amino]benzimidazol-1-yl]-2-pyridyl]-methyl-amino]methyl]cyclopropanecarbonitrile;
1-[2-(2-chloro-5-fluoro-3-pyridyl)-6-[5-[(6-methylpyridazin-3-yl)amino]benzimidazol-1-yl]-3-pyridyl]ethanol;
1-[3-(1-hydroxyethyl)-6-[6-[[6-[(3-methoxyazetidin-1-yl)methyl]pyridazin-3-yl]amino]benzimidazol-1-yl]-2-pyridyl]-5-methyl-pyrazole-3-carbonitrile;
1-[3-(1-hydroxyethyl)-6-[5-[[6-[(3-methoxyazetidin-1-yl)methyl]pyridazin-3-yl]amino]benzimidazol-1-yl]-2-pyridyl]-5-methyl-pyrazole-3-carbonitrile;
1-[6-[5-[(6-methylpyridazin-3-yl)amino]benzimidazol-1-yl]-2-[5-(oxetan-3-yl)-3-(trifluoromethyl)-6,7-dihydro-4H-pyrazolo[4,3-c]pyridin-1-yl]-3-pyridyl]ethanol;
1-[3-(1-hydroxyethyl)-6-[6-[(6-methylpyridazin-3-yl)amino]benzimidazol-1-yl]-2-pyridyl]-3-methyl-pyrazole-4-carbonitrile;
1-[2-[2-(methylamino)-3-pyridyl]-6-[5-[(6-methylpyridazin-3-yl)amino]benzimidazol-1-yl]-3-pyridyl]ethanol;
1-[3-(1-hydroxyethyl)-6-[5-[(6-methylpyridazin-3-yl)amino]benzimidazol-1-yl]-2-pyridyl]-3-methyl-pyrazole-4-carbonitrile;
1-[3-(1-hydroxyethyl)-6-[5-[(6-methylpyridazin-3-yl)amino]benzimidazol-1-yl]-2-pyridyl]-3-methyl-azetidine-3-carbonitrile;
1-[3-(1-hydroxyethyl)-6-[6-[(6-methylpyridazin-3-yl)amino]benzimidazol-1-yl]-2-pyridyl]-3-methyl-azetidine-3-carbonitrile;
1-[3-(1-hydroxyethyl)-6-[6-methoxy-5-[(6-methylpyridazin-3-yl)amino]benzimidazol-1-yl]-2-pyridyl]-5-methyl-pyrazole-3-carbonitrile;
1-[6-[5-[(6-methylpyridazin-3-yl)amino]benzimidazol-1-yl]-2-[2-(1H-pyrazol-4-yl)-3-pyridyl]-3-pyridyl]ethanol;
1-[3-(1-hydroxyethyl)-6-[6-(2-methoxyethoxy)-5-[(6-methylpyridazin-3-yl)amino]benzimidazol-1-yl]-2-pyridyl]-5-methyl-pyrazole-3-carbonitrile;
1-[6-[5-fluoro-6-[[rac-(3R,4S)-4-fluoropyrrolidin-3-yl]amino]benzimidazol-1-yl]-3-(1-hydroxyethyl)pyridin-2-yl]-5-methylpyrazole-3-carbonitrile;
1-[6-[6-[2-(dimethylamino)ethoxy]-5-[(6-methylpyridazin-3-yl)amino]benzimidazol-1-yl]-3-(1-hydroxyethyl)-2-pyridyl]-5-methyl-pyrazole-3-carbonitrile;
1-[6-[5-bromo-6-[4-(oxetan-3-yl)piperazino]benzimidazol-1-yl]-3-(1-hydroxyethyl)-2-pyridyl]-5-methyl-pyrazole-3-carbonitrile;
1-[3-(1-hydroxyethyl)-6-[5-[[6-(3-methoxyazetidine-1-carbonyl)pyridazin-3-yl]amino]benzimidazol-1-yl]-2-pyridyl]-5-methyl-pyrazole-3-carbonitrile;
1-[3-(1-hydroxyethyl)-6-[5-[(6-methylpyridazin-3-yl)amino]-6-pyrrolidin-3-yloxy-benzimidazol-1-yl]-2-pyridyl]-5-methyl-pyrazole-3-carbonitrile;
1-[3-(1-hydroxyethyl)-6-[5-[[6-(2-methoxyethoxy)pyridazin-3-yl]amino]benzimidazol-1-yl]-2-pyridyl]-5-methyl-pyrazole-3-carbonitrile;
1-[2-(1H-benzotriazol-4-yl)-6-[5-[(6-methylpyridazin-3-yl)amino]benzimidazol-1-yl]-3-pyridyl]ethanol;
1-[6-[5-[[6-(2,2-difluoroethoxy)pyridazin-3-yl]amino]benzimidazol-1-yl]-3-(1-hydroxyethyl)-2-pyridyl]-5-methyl-pyrazole-3-carbonitrile;
1-[6-[5-[[6-(difluoromethoxy)pyridazin-3-yl]amino]benzimidazol-1-yl]-3-(1-hydroxyethyl)-2-pyridyl]-5-methyl-pyrazole-3-carbonitrile;
1-[3-(2,2-difluoro-1-hydroxy-ethyl)-6-[5-[(6-methylpyridazin-3-yl)amino]benzimidazol-1-yl]-2-pyridyl]-5-methyl-pyrazole-3-carbonitrile;
1-[3-(2,2-difluoro-1-hydroxy-ethyl)-6-[6-[(6-methylpyridazin-3-yl)amino]benzimidazol-1-yl]-2-pyridyl]-5-methyl-pyrazole-3-carbonitrile;
1-[6-[6-[(1,1-dioxo-1,2-thiazolidin-2-yl)methyl]benzimidazol-1-yl]-3-(1-hydroxyethyl)-2-pyridyl]-5-methyl-pyrazole-3-carbonitrile;
1-[6-[5-[(1,1-dioxo-1,2-thiazolidin-2-yl)methyl]benzimidazol-1-yl]-3-(1-hydroxyethyl)-2-pyridyl]-5-methyl-pyrazole-3-carbonitrile;
3-[3-(1-hydroxyethyl)-6-[5-[(6-methylpyridazin-3-yl)amino]benzimidazol-1-yl]-2-pyridyl]-2-methoxy-phenol;
3-[3-(1-hydroxyethyl)-6-[5-[(6-methylpyridazin-3-yl)amino]benzimidazol-1-yl]-2-pyridyl]furan-2-carbonitrile;
1-[3-(1-hydroxyethyl)-6-[5-[(6-methylpyridazin-3-yl)amino]benzimidazol-1-yl]pyridin-2-yl]-4,5,6,7-tetrahydropyrazolo[4,3-c]pyridine-3-carbonitrile;
1-[6-[5-[[6-[2-(1,1-diketo-1,4-thiazinan-4-yl)ethoxy]pyridazin-3-yl]amino]benzimidazol-1-yl]-3-(1-hydroxyethyl)-2-pyridyl]-5-methyl-pyrazole-3-carbonitrile;
1-[3-(1-hydroxyethyl)-6-[5-[[6-(oxetan-3-yloxy)pyridazin-3-yl]amino]benzimidazol-1-yl]-2-pyridyl]-5-methyl-pyrazole-3-carbonitrile;
1-[6-[5-[(6-methylpyridazin-3-yl)amino]benzimidazol-1-yl]-2-[(3S)-pyrrolidin-3-yl]oxy-3-pyridyl]ethanol;
1-[3-(1-hydroxyethyl)-6-[6-[(2-oxopyrrolidin-1-yl)methyl]benzimidazol-1-yl]-2-pyridyl]-5-methyl-pyrazole-3-carbonitrile;
1-[3-(1-hydroxyethyl)-6-[5-[(2-oxopyrrolidin-1-yl)methyl]benzimidazol-1-yl]-2-pyridyl]-5-methyl-pyrazole-3-carbonitrile;
1-[2-(3-methoxy-5-methyl-pyrazol-1-yl)-6-[5-[(6-methylpyridazin-3-yl)amino]benzimidazol-1-yl]-3-pyridyl]ethanol;
1-[3-(1-hydroxyethyl)-6-[6-[(2-oxo-1-piperidyl)methyl]benzimidazol-1-yl]-2-pyridyl]-5-methyl-pyrazole-3-carbonitrile;
1-[3-(1-hydroxyethyl)-6-[5-[(2-oxo-1-piperidyl)methyl]benzimidazol-1-yl]-2-pyridyl]-5-methyl-pyrazole-3-carbonitrile;
3-[[1-[6-(3-cyano-5-methyl-pyrazol-1-yl)-5-(1-hydroxyethyl)-2-pyridyl]benzimidazol-5-yl]amino]-N,N,6-trimethyl-pyridazine-4-carboxamide; 5-hydroxy-2-[3-(1-hydroxyethyl)-6-[5-[(6-methylpyridazin-3-yl)amino]benzimidazol-1-yl]-2-pyridyl]benzonitrile;
5-[3-(1-hydroxyethyl)-6-[5-[(6-methylpyridazin-3-yl)amino]benzimidazol-1-yl]-2-pyridyl]-3-(trifluoromethyl)-1H-pyridazin-6-one;
2-[3-(1-hydroxyethyl)-6-[5-[(6-methylpyridazin-3-yl)amino]benzimidazol-1-yl]-2-pyridyl]furan-3-carbonitrile;
1-[2-[2-(2,2-difluorocyclopropyl)pyrazol-3-yl]-6-[5-[(6-methylpyridazin-3-yl)amino]benzimidazol-1-yl]-3-pyridyl]ethanol;
1-[3-(1-hydroxyethyl)-6-[5-[[6-(oxetan-3-yl)pyridazin-3-yl]amino]benzimidazol-1-yl]-2-pyridyl]-5-methyl-pyrazole-3-carbonitrile;
(1S)-1-[2-[3-(difluoromethyl)-5-methyl-pyrazol-1-yl]-6-[5-[(6-methylpyridazin-3-yl)amino]benzimidazol-1-yl]-3-pyridyl]ethanol;
(1R)-1-[2-[3-(difluoromethyl)-5-methyl-pyrazol-1-yl]-6-[5-[(6-methylpyridazin-3-yl)amino]benzimidazol-1-yl]-3-pyridyl]ethanol;
1-[2-[5-(difluoromethyl)-3-methyl-pyrazol-1-yl]-6-[5-[(6-methylpyridazin-3-yl)amino]benzimidazol-1-yl]-3-pyridyl]ethanol;
1-[3-(1-hydroxy-2-methoxy-ethyl)-6-[5-[(6-methylpyridazin-3-yl)amino]benzimidazol-1-yl]-2-pyridyl]-5-methyl-pyrazole-3-carbonitrile;
1-[3-(1-hydroxy-2-methoxy-ethyl)-6-[6-[(6-methylpyridazin-3-yl)amino]benzimidazol-1-yl]-2-pyridyl]-5-methyl-pyrazole-3-carbonitrile;
1-[6-[6-fluoro-5-[(6-methylpyridazin-3-yl)amino]benzimidazol-1-yl]-3-[(1S)-1-hydroxyethyl]-2-pyridyl]-5-methyl-pyrazole-3-carbonitrile;
1-[6-[6-fluoro-5-[(6-methylpyridazin-3-yl)amino]benzimidazol-1-yl]-3-[(1R)-1-hydroxyethyl]-2-pyridyl]-5-methyl-pyrazole-3-carbonitrile;
1-[5-chloro-3-(1-hydroxyethyl)-6-[5-[(6-methylpyridazin-3-yl)amino]benzimidazol-1-yl]-2-pyridyl]pyrazole-3-carbonitrile;
1-[6-[5-[(6-methylpyridazin-3-yl)amino]benzimidazol-1-yl]-2-[5-methyl-1-(2,2,2-trifluoroethyl)pyrazol-4-yl]-3-pyridyl]ethanol;
1-[6-[5-[(6-chloro-4-methoxy-pyridazin-3-yl)amino]benzimidazol-1-yl]-3-(1-hydroxyethyl)-2-pyridyl]-5-methyl-pyrazole-3-carbonitrile;
rac-(3R,5S)-1-[3-(1-hydroxyethyl)-6-[5-[(6-methylpyridazin-3-yl)amino]benzimidazol-1-yl]-2-pyridyl]-5-methyl-pyrrolidine-3-carbonitrile;
1-[5-chloro-3-(1-hydroxyethyl)-6-[5-[(6-methylpyridazin-3-yl)amino]benzimidazol-1-yl]-2-pyridyl]-5-methyl-pyrazole-3-carbonitrile;
5-[3-(1-hydroxyethyl)-6-[5-[(6-methylpyridazin-3-yl)amino]benzimidazol-1-yl]-2-pyridyl]-2-methyl-pyrazole-3-carbonitrile;
1-[3-[(1R)-1-hydroxyethyl]-6-[6-[(6-methylpyridazin-3-yl)amino]benzimidazol-1-yl]-2-pyridyl]-5-methyl-pyrazole-3-carbonitrile;
1-[2-[3,5-bis(difluoromethyl)pyrazol-1-yl]-6-[5-[(6-methylpyridazin-3-yl)amino]benzimidazol-1-yl]pyridin-3-yl]ethanol;
1-[3-(1-hydroxyethyl)-6-[5-[[6-(trifluoromethyl)pyridazin-3-yl]amino]benzimidazol-1-yl]-2-pyridyl]-5-methyl-pyrazole-3-carbonitrile;
1-[2-[2-ethyl-5-(trifluoromethyl)pyrazol-3-yl]-6-[5-[(6-methylpyridazin-3-yl)amino]benzimidazol-1-yl]-3-pyridyl]ethanol;
rac-(1S)-1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanol;
rac-(1R)-1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanol;
1-[3-(1-hydroxyethyl)-6-[5-[(6-methylpyridazin-3-yl)amino]benzimidazol-1-yl]-2-pyridyl]triazole-4-carbonitrile;
(3S,5R)-1-[3-(1-hydroxyethyl)-6-[5-[(6-methylpyridazin-3-yl)amino]benzimidazol-1-yl]-2-pyridyl]-5-methyl-pyrrolidine-3-carbonitrile;
5-[3-(1-hydroxyethyl)-6-[5-[(6-methylpyridazin-3-yl)amino]benzimidazol-1-yl]-2-pyridyl]-1-methyl-pyrazole-3-carbonitrile;
2-[3-(1-hydroxyethyl)-6-[5-[(6-methylpyridazin-3-yl)amino]benzimidazol-1-yl]-2-pyridyl]triazole-4-carbonitrile;
1-[6-(3-cyano-5-methylpyrazol-1-yl)-5-(1-hydroxyethyl)pyridin-2-yl]benzimidazole-5-carbonitrile;
1-[2-(1-ethyl-3-methyl-pyrazol-4-yl)-6-[5-[(6-methylpyridazin-3-yl)amino]benzimidazol-1-yl]-3-pyridyl]ethanol;
1-[2-[1-(2-methoxyethyl)-3-methyl-pyrazol-4-yl]-6-[5-[(6-methylpyridazin-3-yl)amino]benzimidazol-1-yl]-3-pyridyl]ethanol;
1-[2-[1-(cyclopropylmethyl)-3-methyl-pyrazol-4-yl]-6-[5-[(6-methylpyridazin-3-yl)amino]benzimidazol-1-yl]-3-pyridyl]ethanol;
5-methyl-1-[3-methylol-6-[6-[(6-methylpyridazin-3-yl)amino]benzimidazol-1-yl]-2-pyridyl]pyrazole-3-carbonitrile;
5-methyl-1-[3-methylol-6-[5-[(6-methylpyridazin-3-yl)amino]benzimidazol-1-yl]-2-pyridyl]pyrazole-3-carbonitrile;
1-[2-(1-ethyl-5-methyl-pyrazol-4-yl)-6-[5-[(6-methylpyridazin-3-yl)amino]benzimidazol-1-yl]-3-pyridyl]ethanol;
1-[2-[1-(cyclopropylmethyl)-5-methyl-pyrazol-4-yl]-6-[5-[(6-methylpyridazin-3-yl)amino]benzimidazol-1-yl]-3-pyridyl]ethanol;
1-[2-[1-(cyclobutylmethyl)-3-methyl-pyrazol-4-yl]-6-[5-[(6-methylpyridazin-3-yl)amino]benzimidazol-1-yl]-3-pyridyl]ethanol;
1-[2-[1-(cyclobutylmethyl)-5-methyl-pyrazol-4-yl]-6-[5-[(6-methylpyridazin-3-yl)amino]benzimidazol-1-yl]-3-pyridyl]ethanol;
   or a pharmaceutically acceptable salt thereof.

68. A compound according to any one of embodiments 1 to 67 selected from
1-[3-(1-hydroxyethyl)-6-[5-[(6-methylpyridazin-3-yl)amino]benzimidazol-1-yl]-2-pyridyl]-5-methyl-pyrazole-3-carbonitrile;
1-[3-(1-hydroxyethyl)-6-[5-(6-methylpyridazin-3-yl)oxybenzimidazol-1-yl]-2-pyridyl]-5-methyl-pyrazole-3-carbonitrile;
1-[3-(1-hydroxyethyl)-6-[5-[(2-methylpyrimidin-5-yl)amino]benzimidazol-1-yl]-2-pyridyl]-5-methyl-pyrazole-3-carbonitrile;
1-[3-(1-hydroxyethyl)-6-[5-[(6-methoxypyridazin-3-yl)amino]benzimidazol-1-yl]-2-pyridyl]-5-methyl-pyrazole-3-carbonitrile;
1-[3-(1-hydroxyethyl)-6-[5-(pyridazin-3-ylamino)benzimidazol-1-yl]-2-pyridyl]-5-methyl-pyrazole-3-carbonitrile;
1-[6-[5-[[6-(difluoromethyl)pyridazin-3-yl]amino]benzimidazol-1-yl]-3-(1-hydroxyethyl)-2-pyridyl]-5-methyl-pyrazole-3-carbonitrile;
1-[6-[6-fluoro-5-[(6-methylpyridazin-3-yl)amino]benzimidazol-1-yl]-3-(1-hydroxyethyl)-2-pyridyl]-5-methyl-pyrazole-3-carbonitrile;
1-[6-[5-[(6-methylpyridazin-3-yl)amino]benzimidazol-1-yl]-2-[3-(trifluoromethyl)-4,5,6,7-tetrahydropyrazolo[4,3-c]pyridin-1-yl]-3-pyridyl]ethanol;
2-[3-(1-hydroxyethyl)-6-[5-[(6-methylpyridazin-3-yl)amino]benzimidazol-1-yl]-2-pyridyl]-5-methyl-pyrazole-3-carbonitrile;
rac-(3R)-1-[3-(1-hydroxyethyl)-6-[5-[(6-methylpyridazin-3-yl)amino]benzimidazol-1-yl]-2-pyridyl]pyrrolidine-3-carbonitrile;
1-[2-(3,5-dimethylpyrazol-1-yl)-6-[5-[(6-methylpyridazin-3-yl)amino]benzimidazol-1-yl]-3-pyridyl]ethanol;
5-methyl-1-[6-[5-[(6-methylpyridazin-3-yl)amino]benzimidazol-1-yl]-3-[rac-(1R)-1-hydroxyethyl]-2-pyridyl]pyrazole-3-carbonitrile;
5-methyl-1-[6-[5-[(6-methylpyridazin-3-yl)amino]benzimidazol-1-yl]-3-[rac-(1S)-1-hydroxyethyl]-2-pyridyl]pyrazole-3-carbonitrile;
1-[3-(1-hydroxyethyl)-6-[5-[(6-methylpyridazin-3-yl)amino]benzimidazol-1-yl]-2-pyridyl]pyrazole-3-carbonitrile;
5-[3-(1-hydroxyethyl)-6-[5-[(6-methylpyridazin-3-yl)amino]benzimidazol-1-yl]-2-pyridyl]pyridine-3-carbonitrile;
1-[3-(1-hydroxyethyl)-6-[5-[(6-methylpyridazin-3-yl)amino]benzimidazol-1-yl]-2-pyridyl]-3-methyl-pyrrolidine-3-carbonitrile;
1-[6-[5-[(6-methylpyridazin-3-yl)amino]benzimidazol-1-yl]-2-[2-methyl-5-(trifluoromethyl)pyrazol-3-yl]-3-pyridyl]ethanol;
3-(1-hydroxyethyl)-6-[5-[(6-methylpyridazin-3-yl)amino]benzimidazol-1-yl]-N-(2,2,2-trifluoroethyl)pyridine-2-carboxamide;
1-[6-[5-[(6-methylpyridazin-3-yl)amino]benzimidazol-1-yl]-2-[rac-(3aR,6aS)-2,3,3a,5,6,6a-hexahydro-1H-pyrrolo[3,2-b]pyrrol-4-yl]-3-pyridyl]ethanol;
1-[2-[3-(difluoromethyl)-5-methyl-pyrazol-1-yl]-6-[5-[(6-methylpyridazin-3-yl)amino]benzimidazol-1-yl]-3-pyridyl]ethanol;
1-[2-(3-ethoxy-5-methyl-pyrazol-1-yl)-6-[5-[(6-methylpyridazin-3-yl)amino]benzimidazol-1-yl]-3-pyridyl]ethanol;
4-[3-(1-hydroxyethyl)-6-[5-[(6-methylpyridazin-3-yl)amino]benzimidazol-1-yl]-2-pyridyl]-1-(2,2,2-trifluoroethyl)piperazin-2-one;
formic acid; 1-[[3-(1-hydroxyethyl)-6-[5-[(6-methylpyridazin-3-yl)amino]benzimidazol-1-yl]-2-pyridyl]oxymethyl]cyclopropanecarbonitrile;
1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanol;
1-[2-(5-methyl-3-methylsulfonyl-pyrazol-1-yl)-6-[5-[(6-methylpyridazin-3-yl)amino]benzimidazol-1-yl]-3-pyridyl]ethanol;
5-methyl-1-[6-[5-[(6-methylpyridazin-3-yl)amino]benzimidazol-1-yl]-3-(2,2,2-trifluoro-1-hydroxy-ethyl)-2-pyridyl]pyrazole-3-carbonitrile;
1-[2-[1-(difluoromethyl)-3-methyl-pyrazol-4-yl]-6-[5-[(6-methylpyridazin-3-yl)amino]benzimidazol-1-yl]-3-pyridyl]ethanol;
[2-(3-methoxy-5-methyl-pyrazol-1-yl)-6-[5-[(6-methylpyridazin-3-yl)amino]benzimidazol-1-yl]-3-pyridyl]methanol;
1-[3-(1-hydroxyethyl)-6-[5-[(4-methoxypyridazin-3-yl)amino]benzimidazol-1-yl]-2-pyridyl]-5-methyl-pyrazole-3-carbonitrile;
1-[2-[2-(difluoromethoxy)-5-methyl-4-pyridyl]-6-[5-[(6-methylpyridazin-3-yl)amino]benzimidazol-1-yl]-3-pyridyl]ethanol;
1-[3-(1-hydroxyethyl)-6-[5-[(6-methylpyridazin-3-yl)amino]benzimidazol-1-yl]-2-pyridyl]-4-methyl-pyrazole-3-carbonitrile;
1-[6-[5-[(6-methylpyridazin-3-yl)amino]benzimidazol-1-yl]-2-[5-methyl-3-(trifluoromethyl)-6,7-dihydro-4H-pyrazolo[4,3-c]pyridin-1-yl]-3-pyridyl]ethanol;
1-[3-(1-hydroxyethyl)-6-[5-[[6-[(3-methoxyazetidin-1-yl)methyl]pyridazin-3-yl]amino]benzimidazol-1-yl]-2-pyridyl]-5-methyl-pyrazole-3-carbonitrile;
1-[6-[5-[(6-methylpyridazin-3-yl)amino]benzimidazol-1-yl]-2-[5-(oxetan-3-yl)-3-(trifluoromethyl)-6,7-dihydro-4H-pyrazolo[4,3-c]pyridin-1-yl]-3-pyridyl]ethanol;
1-[3-(1-hydroxyethyl)-6-[5-[(6-methylpyridazin-3-yl)amino]benzimidazol-1-yl]-2-pyridyl]-3-methyl-pyrazole-4-carbonitrile;
1-[3-(1-hydroxyethyl)-6-[6-methoxy-5-[(6-methylpyridazin-3-yl)amino]benzimidazol-1-yl]-2-pyridyl]-5-methyl-pyrazole-3-carbonitrile;
1-[3-(1-hydroxyethyl)-6-[6-(2-methoxyethoxy)-5-[(6-methylpyridazin-3-yl)amino]benzimidazol-1-yl]-2-pyridyl]-5-methyl-pyrazole-3-carbonitrile;
1-[6-[6-[2-(dimethylamino)ethoxy]-5-[(6-methylpyridazin-3-yl)amino]benzimidazol-1-yl]-3-(1-hydroxyethyl)-2-pyridyl]-5-methyl-pyrazole-3-carbonitrile;
1-[3-(1-hydroxyethyl)-6-[5-[(6-methylpyridazin-3-yl)amino]-6-pyrrolidin-3-yloxy-benzimidazol-1-yl]-2-pyridyl]-5-methyl-pyrazole-3-carbonitrile;
1-[3-(1-hydroxyethyl)-6-[5-[[6-(2-methoxyethoxy)pyridazin-3-yl]amino]benzimidazol-1-yl]-2-pyridyl]-5-methyl-pyrazole-3-carbonitrile;
1-[6-[5-[[6-(2,2-difluoroethoxy)pyridazin-3-yl]amino]benzimidazol-1-yl]-3-(1-hydroxyethyl)-2-pyridyl]-5-methyl-pyrazole-3-carbonitrile;
1-[6-[5-[[6-(difluoromethoxy)pyridazin-3-yl]amino]benzimidazol-1-yl]-3-(1-hydroxyethyl)-2-pyridyl]-5-methyl-pyrazole-3-carbonitrile;
1-[3-(2,2-difluoro-1-hydroxy-ethyl)-6-[5-[(6-methylpyridazin-3-yl)amino]benzimidazol-1-yl]-2-pyridyl]-5-methyl-pyrazole-3-carbonitrile;
1-[3-(1-hydroxyethyl)-6-[5-[(6-methylpyridazin-3-yl)amino]benzimidazol-1-yl]pyridin-2-yl]-4,5,6,7-tetrahydropyrazolo[4,3-c]pyridine-3-carbonitrile;
1-[6-[5-[[6-[2-(1,1-diketo-1,4-thiazinan-4-yl)ethoxy]pyridazin-3-yl]amino]benzimidazol-1-yl]-3-(1-hydroxyethyl)-2-pyridyl]-5-methyl-pyrazole-3-carbonitrile;
1-[3-(1-hydroxyethyl)-6-[5-[[6-(oxetan-3-yloxy)pyridazin-3-yl]amino]benzimidazol-1-yl]-2-pyridyl]-5-methyl-pyrazole-3-carbonitrile;
1-[2-(3-methoxy-5-methyl-pyrazol-1-yl)-6-[5-[(6-methylpyridazin-3-yl)amino]benzimidazol-1-yl]-3-pyridyl]ethanol;
3-[[1-[6-(3-cyano-5-methyl-pyrazol-1-yl)-5-(1-hydroxyethyl)-2-pyridyl]benzimidazol-5-yl]amino]-N,N,6-trimethyl-pyridazine-4-carboxamide;
1-[3-(1-hydroxyethyl)-6-[5-[[6-(oxetan-3-yl)pyridazin-3-yl]amino]benzimidazol-1-yl]-2-pyridyl]-5-methyl-pyrazole-3-carbonitrile;
(1S)-1-[2-[3-(difluoromethyl)-5-methyl-pyrazol-1-yl]-6-[5-[(6-methylpyridazin-3-yl)amino]benzimidazol-1-yl]-3-pyridyl]ethanol;
(1R)-1-[2-[3-(difluoromethyl)-5-methyl-pyrazol-1-yl]-6-[5-[(6-methylpyridazin-3-yl)amino]benzimidazol-1-yl]-3-pyridyl]ethanol;
1-[2-[5-(difluoromethyl)-3-methyl-pyrazol-1-yl]-6-[5-[(6-methylpyridazin-3-yl)amino]benzimidazol-1-yl]-3-pyridyl]ethanol;
1-[3-(1-hydroxy-2-methoxy-ethyl)-6-[5-[(6-methylpyridazin-3-yl)amino]benzimidazol-1-yl]-2-pyridyl]-5-methyl-pyrazole-3-carbonitrile;
1-[6-[6-fluoro-5-[(6-methylpyridazin-3-yl)amino]benzimidazol-1-yl]-3-[(1S)-1-hydroxyethyl]-2-pyridyl]-5-methyl-pyrazole-3-carbonitrile;
1-[6-[6-fluoro-5-[(6-methylpyridazin-3-yl)amino]benzimidazol-1-yl]-3-[(1R)-1-hydroxyethyl]-2-pyridyl]-5-methyl-pyrazole-3-carbonitrile;
1-[6-[5-[(6-methylpyridazin-3-yl)amino]benzimidazol-1-yl]-2-[5-methyl-1-(2,2,2-trifluoroethyl)pyrazol-4-yl]-3-pyridyl]ethanol;
1-[6-[5-[(6-chloro-4-methoxy-pyridazin-3-yl)amino]benzimidazol-1-yl]-3-(1-hydroxyethyl)-2-pyridyl]-5-methyl-pyrazole-3-carbonitrile;
rac-(3R,5S)-1-[3-(1-hydroxyethyl)-6-[5-[(6-methylpyridazin-3-yl)amino]benzimidazol-1-yl]-2-pyridyl]-5-methyl-pyrrolidine-3-carbonitrile;
5-[3-(1-hydroxyethyl)-6-[5-[(6-methylpyridazin-3-yl)amino]benzimidazol-1-yl]-2-pyridyl]-2-methyl-pyrazole-3-carbonitrile;
1-[2-[3,5-bis(difluoromethyl)pyrazol-1-yl]-6-[5-[(6-methylpyridazin-3-yl)amino]benzimidazol-1-yl]pyridin-3-yl]ethanol;
1-[3-(1-hydroxyethyl)-6-[5-[[6-(trifluoromethyl)pyridazin-3-yl]amino]benzimidazol-1-yl]-2-pyridyl]-5-methyl-pyrazole-3-carbonitrile;
1-[2-[2-ethyl-5-(trifluoromethyl)pyrazol-3-yl]-6-[5-[(6-methylpyridazin-3-yl)amino]benzimidazol-1-yl]-3-pyridyl]ethanol;
rac-(1S)-1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanol;
rac-(1R)-1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanol;
1-[2-(1-ethyl-3-methyl-pyrazol-4-yl)-6-[5-[(6-methylpyridazin-3-yl)amino]benzimidazol-1-yl]-3-pyridyl]ethanol;
1-[2-[1-(2-methoxyethyl)-3-methyl-pyrazol-4-yl]-6-[5-[(6-methylpyridazin-3-yl)amino]benzimidazol-1-yl]-3-pyridyl]ethanol;
1-[2-[1-(cyclopropylmethyl)-3-methyl-pyrazol-4-yl]-6-[5-[(6-methylpyridazin-3-yl)amino]benzimidazol-1-yl]-3-pyridyl]ethanol;
5-methyl-1-[3-methylol-6-[5-[(6-methylpyridazin-3-yl)amino]benzimidazol-1-yl]-2-pyridyl]pyrazole-3-carbonitrile;
1-[2-(1-ethyl-5-methyl-pyrazol-4-yl)-6-[5-[(6-methylpyridazin-3-yl)amino]benzimidazol-1-yl]-3-pyridyl]ethanol;
1-[2-[1-(cyclopropylmethyl)-5-methyl-pyrazol-4-yl]-6-[5-[(6-methylpyridazin-3-yl)amino]benzimidazol-1-yl]-3-pyridyl]ethanol;
1-[2-[1-(cyclobutylmethyl)-3-methyl-pyrazol-4-yl]-6-[5-[(6-methylpyridazin-3-yl)amino]benzimidazol-1-yl]-3-pyridyl]ethanol;
1-[2-[1-(cyclobutylmethyl)-5-methyl-pyrazol-4-yl]-6-[5-[(6-methylpyridazin-3-yl)amino]benzimidazol-1-yl]-3-pyridyl]ethanol;
   or a pharmaceutically acceptable salt thereof.

69. A compound according to any one of embodiments 1 to 68, selected from
1-[3-(1-hydroxyethyl)-6-[5-[(6-methylpyridazin-3-yl)amino]-6-(trifluoromethyl)benzimidazol-1-yl]-2-pyridyl]-5-methyl-pyrazole-3-carbonitrile;
1-[3-[(1S)-1-hydroxyethyl]-6-[5-[(6-methylpyridazin-3-yl)amino]benzimidazol-1-yl]-2-pyridyl]-5-methyl-pyrazole-3-carboximidic acid methyl ester;
1-[2-[1-(2,2-difluorocyclopropyl)-3-methyl-pyrazol-4-yl]-6-[5-[(6-methylpyridazin-3-yl)amino]benzimidazol-1-yl]-3-pyridyl]ethanol;
1-[2-[1-(2-methoxypropyl)-3-methyl-pyrazol-4-yl]-6-[5-[(6-methylpyridazin-3-yl)amino]benzimidazol-1-yl]-3-pyridyl]ethanol;
1-[2-[1-(2-methoxypropyl)-5-methyl-pyrazol-4-yl]-6-[5-[(6-methylpyridazin-3-yl)amino]benzimidazol-1-yl]-3-pyridyl]ethanol;
1-[3-[(1S)-1-hydroxyethyl]-6-[6-[(6-methylpyridazin-3-yl)amino]benzimidazol-1-yl]-2-pyridyl]-5-methyl-pyrazole-3-carbonitrile;
1-[2-[1-(2,2-difluoropropyl)-3-methyl-pyrazol-4-yl]-6-[5-[(6-methylpyridazin-3-yl)amino]benzimidazol-1-yl]-3-pyridyl]ethanol;
1-[3-(1-hydroxyethyl)-6-[5-[(6-methylpyridazin-3-yl)amino]-6-(oxetan-3-yloxy)benzimidazol-1-yl]-2-pyridyl]-5-methyl-pyrazole-3-carbonitrile;
1-[2-(2-ethyl-5-methyl-4-pyridyl)-6-[5-[(6-methylpyridazin-3-yl)amino]benzimidazol-1-yl]-3-pyridyl]ethanol;
1-[2-[1-(2,2-difluoropropyl)-5-methyl-pyrazol-4-yl]-6-[5-[(6-methylpyridazin-3-yl)amino]benzimidazol-1-yl]-3-pyridyl]ethanol;
1-[2-(2-methoxy-5-methyl-4-pyridyl)-6-[5-[(6-methylpyridazin-3-yl)amino]benzimidazol-1-yl]-3-pyridyl]ethanol;
(1S)-1-[2-[3,5-bis(difluoromethyl)pyrazol-1-yl]-6-[5-[(6-methylpyridazin-3-yl)amino]benzimidazol-1-yl]-3-pyridyl]ethanol;
(1R)-1-[2-[3,5-bis(difluoromethyl)pyrazol-1-yl]-6-[5-[(6-methylpyridazin-3-yl)amino]benzimidazol-1-yl]-3-pyridyl]ethanol;
1-[2-(2-cyclopropyl-5-methyl-4-pyridyl)-6-[5-[(6-methylpyridazin-3-yl)amino]benzimidazol-1-yl]-3-pyridyl]ethanol;
1-[2-[3-(difluoromethoxy)-5-methyl-pyrazol-1-yl]-6-[5-[(6-methylpyridazin-3-yl)amino]benzimidazol-1-yl]-3-pyridyl]ethanol;
1-[3-(1-hydroxyethyl)-6-[6-[(6-methylpyridazin-3-yl)amino]benzimidazol-1-yl]-2-pyridyl]-5-methyl-6,7-dihydro-4˜{H}-pyrazolo[4,3-c]pyridine-3-carbonitrile;
1-[3-(1-hydroxyethyl)-6-[5-[(6-methyl-4-methylsulfonyl-pyridazin-3-yl)amino]benzimidazol-1-yl]-2-pyridyl]-5-methyl-pyrazole-3-carbonitrile;
1-[3-(1-hydroxyethyl)-6-[5-[[6-[2-[2-[2-[2-[2-[2-(2-methoxyethoxy)ethoxy]ethoxy]ethoxy]ethoxy]ethoxy]ethoxy]pyridazin-3-yl]amino]benzimidazol-1-yl]-2-pyridyl]-5-methyl-pyrazole-3-carbonitrile;
1-[3-(1-hydroxyethyl)-6-[6-[(6-methyl-3-pyridyl)amino]-5-(2-morpholinoethoxy)benzimidazol-1-yl]-2-pyridyl]-5-methyl-pyrazole-3-carbonitrile;
1-[3-(1-hydroxyethyl)-6-[6-(2-oxa-5-azaspiro[3.4]octan-5-yl)benzimidazol-1-yl]-2-pyridyl]-5-methyl-pyrazole-3-carbonitrile;
1-[3-(1-hydroxyethyl)-6-[5-(2-oxa-5-azaspiro[3.4]octan-5-yl)benzimidazol-1-yl]-2-pyridyl]-5-methyl-pyrazole-3-carbonitrile;
1-[3-(1-hydroxyethyl)-6-[5-[(6-methyl-3-pyridyl)amino]-6-(2-morpholinoethoxy)benzimidazol-1-yl]-2-pyridyl]-5-methyl-pyrazole-3-carbonitrile;
1-[2-[3-(difluoromethyl)pyrrolidin-1-yl]-6-[5-[(6-methylpyridazin-3-yl)amino]benzimidazol-1-yl]-3-pyridyl]ethanol;
1-[2-(2,2-difluoro-5-azaspiro[2.4]heptan-5-yl)-6-[5-[(6-methylpyridazin-3-yl)amino]benzimidazol-1-yl]-3-pyridyl]ethanol;
(1S)-1-[2-[3-(difluoromethyl)-5-methyl-pyrazol-1-yl]-6-[6-fluoro-5-[(6-methylpyridazin-3-yl)amino]benzimidazol-1-yl]-3-pyridyl]ethanol;
1-[6-[6-fluoro-5-[(6-methylpyridazin-3-yl)amino]benzimidazol-1-yl]-2-[3-methyl-1-(2,2,2-trifluoroethyl)pyrazol-4-yl]-3-pyridyl]ethanol; 5-(difluoromethyl)-2-[3-(1-hydroxyethyl)-6-[5-[(6-methylpyridazin-3-yl)amino]benzimidazol-1-yl]-2-pyridyl]pyrazole-3-carbonitrile;
1-[3-(1-hydroxyethyl)-6-[5-[[6-[1-(oxetan-3-yl)pyrrolidin-2-yl]pyridazin-3-yl]amino]benzimidazol-1-yl]-2-pyridyl]-5-methyl-pyrazole-3-carbonitrile;
7,7-difluoro-1-[3-(1-hydroxyethyl)-6-[5-[(6-methylpyridazin-3-yl)amino]benzimidazol-1-yl]-2-pyridyl]-5,6-dihydro-4˜{H}-pyrazolo[4,3-c]pyridine-3-carbonitrile;
5-(difluoromethyl)-1-[3-(1-hydroxyethyl)-6-[5-[(6-methylpyridazin-3-yl)amino]benzimidazol-1-yl]-2-pyridyl]pyrazole-3-carbonitrile;
(1S)-1-[2-[3-(difluoromethyl)-5-methyl-pyrazol-1-yl]-6-[6-fluoro-5-(pyridazin-3-ylamino)benzimidazol-1-yl]-3-pyridyl]ethanol;
1-[6-[5-[(6-methylpyridazin-3-yl)amino]benzimidazol-1-yl]-2-[5-methyl-2-(2,2,2-trifluoroethyl)triazol-4-yl]-3-pyridyl]ethanol;
1-[3-(1-hydroxyethyl)-6-[5-[(6-methylpyridazin-3-yl)amino]benzimidazol-1-yl]-2-pyridyl]-4,5-dimethyl-pyrazole-3-carbonitrile;
1-[3-(1-hydroxyethyl)-6-[5-[(4-methyl-2,3-dihydropyridazino[4,5-b][1,4]oxazin-8-yl)amino]benzimidazol-1-yl]-2-pyridyl]-5-methyl-pyrazole-3-carbonitrile;
1-[6-[5-[(6-methylpyridazin-3-yl)amino]benzimidazol-1-yl]-2-[5-methyl-1-(2,2,2-trifluoroethyl)triazol-4-yl]-3-pyridyl]ethanol;
1-[1-[6-(3-cyano-5-methyl-pyrazol-1-yl)-5-(1-hydroxyethyl)-2-pyridyl]benzimidazol-5-yl]-N,N-dimethyl-pyrrolidine-2-carboxamide;
1-[2-[1-(2,2-difluoroethyl)-3-methyl-pyrazol-4-yl]-6-[5-[(6-methylpyridazin-3-yl)amino]benzimidazol-1-yl]-3-pyridyl]ethanol;
1-[6-[5-fluoro-6-[(6-methylpyridazin-3-yl)amino]benzimidazol-1-yl]-3-(hydroxymethyl)-2-pyridyl]-5-methyl-pyrazole-3-carbonitrile; [6-[5-[(6-methylpyridazin-3-yl)amino]benzimidazol-1-yl]-2-[3-methyl-1-(2,2,2-trifluoroethyl)pyrazol-4-yl]-3-pyridyl]methanol; [6-[5-[(6-methylpyridazin-3-yl)amino]benzimidazol-1-yl]-2-[5-methyl-1-(2,2,2-trifluoroethyl)pyrazol-4-yl]-3-pyridyl]methanol; [2-[3-(difluoromethyl)-5-methyl-pyrazol-1-yl]-6-[5-(7,8-dihydro-5H-pyrano[4,3-c]pyridazin-3-ylamino)benzimidazol-1-yl]-3-pyridyl]methanol; [6-[5-[(4aS,7aR)-4-methyl-2,3,4a,5,7,7a-hexahydropyrrolo[3,4-b][1,4]oxazin-6-yl]benzimidazol-1-yl]-2-[3-(difluoromethyl)-5-methyl-pyrazol-1-yl]-3-pyridyl]methanol; [2-[3-(difluoromethyl)-5-methyl-pyrazol-1-yl]-6-[6-fluoro-5-[(6-methylpyridazin-3-yl)amino]benzimidazol-1-yl]-3-pyridyl]methanol;
1-[6-[5-[(6-methylpyridazin-3-yl)amino]benzimidazol-1-yl]-2-[2-(trifluoromethyl)morpholin-4-yl]-3-pyridyl]ethanol;
3-[3-(1-hydroxyethyl)-6-[5-[(6-methylpyridazin-3-yl)amino]benzimidazol-1-yl]-2-pyridyl]-3-azabicyclo[3.1.0]hexane-1-carbonitrile;
1-[2-(5,5-difluoro-2-azabicyclo[2.2.1]heptan-2-yl)-6-[5-[(6-methylpyridazin-3-yl)amino]benzimidazol-1-yl]-3-pyridyl]ethanol;
1-[2-[2-(difluoromethyl)morpholin-4-yl]-6-[5-[(6-methylpyridazin-3-yl)amino]benzimidazol-1-yl]-3-pyridyl]ethanol;
1-[2-(6,6-difluoro-2-azabicyclo[2.2.1]heptan-2-yl)-6-[5-[(6-methylpyridazin-3-yl)amino]benzimidazol-1-yl]-3-pyridyl]ethanol;
1-[2-[7-(difluoromethyl)-5-azaspiro[2.4]heptan-5-yl]-6-[5-[(6-methylpyridazin-3-yl)amino]benzimidazol-1-yl]-3-pyridyl]ethanol;
1-[2-(6,6-difluoro-3-azabicyclo[3.2.0]heptan-3-yl)-6-[5-[(6-methylpyridazin-3-yl)amino]benzimidazol-1-yl]-3-pyridyl]ethanol;
N-[1-[6-(3-cyano-5-methyl-pyrazol-1-yl)-5-(1-hydroxyethyl)-2-pyridyl]benzimidazol-5-yl]cyclopropanecarboxamide;
N-[3-[6-(3-cyano-5-methyl-pyrazol-1-yl)-5-(1-hydroxyethyl)-2-pyridyl]benzimidazol-5-yl]cyclopropanecarboxamide;
1-[2-(4,7-diazaspiro[2.5]octan-7-yl)-6-[5-[(6-methylpyridazin-3-yl)amino]benzimidazol-1-yl]-3-pyridyl]ethanol;
1-[6-[5-[(3aS,6aS)-6-oxo-2,3,3a,4,5,6a-hexahydropyrrolo[2,3-c]pyrrol-1-yl]benzimidazol-1-yl]-3-(1-hydroxyethyl)-2-pyridyl]-5-methyl-pyrazole-3-carbonitrile;
1-[6-[5-[(6-methylpyridazin-3-yl)amino]benzimidazol-1-yl]-2-[3-(trifluoromethyl)piperazin-1-yl]-3-pyridyl]ethanol;
1-[6-[5-[(6-methylpyridazin-3-yl)amino]benzimidazol-1-yl]-2-[5-methyl-2-(trifluoromethyl)-4-pyridyl]-3-pyridyl]ethanol;
1-[3-(1-hydroxyethyl)-6-[5-[(2-keto-1-methyl-3-pyridyl)amino]benzimidazol-1-yl]-2-pyridyl]-5-methyl-pyrazole-3-carbonitrile;
1-[3-(1-hydroxyethyl)-6-[5-(3-methoxy-1-methyl-pyrazol-4-yl)benzimidazol-1-yl]-2-pyridyl]-5-methyl-pyrazole-3-carbonitrile;
1-[2-[2-(difluoromethyl)-5-methyl-4-pyridyl]-6-[5-[(6-methylpyridazin-3-yl)amino]benzimidazol-1-yl]-3-pyridyl]ethanol;
1-[3-(1-hydroxyethyl)-6-[6-(1-methyl-2-oxo-pyrimidin-4-yl)benzimidazol-1-yl]-2-pyridyl]-5-methyl-pyrazole-3-carbonitrile;
1-[2-[3-(difluoromethyl)-5-methyl-pyrazol-1-yl]-6-[6-fluoro-5-[[6-(2-methyl-1,3-dioxolan-2-yl)pyridazin-3-yl]amino]benzimidazol-1-yl]-3-pyridyl]ethanol;
1-[3-(1-hydroxyethyl)-6-[5-(1-methyl-2-oxo-pyrimidin-4-yl)benzimidazol-1-yl]-2-pyridyl]-5-methyl-pyrazole-3-carbonitrile;
1-[3-(1-hydroxyethyl)-6-[6-(6-methylpyridazin-3-yl)benzimidazol-1-yl]-2-pyridyl]-5-methyl-pyrazole-3-carbonitrile;
1-[3-(1-hydroxyethyl)-6-[5-(6-methylpyridazin-3-yl)benzimidazol-1-yl]-2-pyridyl]-5-methyl-pyrazole-3-carbonitrile;
1-[6-[6-[(2S)-2-cyanopyrrolidin-1-yl]benzimidazol-1-yl]-3-(1-hydroxyethyl)-2-pyridyl]-5-methyl-pyrazole-3-carbonitrile;
1-[6-[5-[(2S)-2-cyanopyrrolidin-1-yl]benzimidazol-1-yl]-3-(1-hydroxyethyl)-2-pyridyl]-5-methyl-pyrazole-3-carbonitrile;
1-[6-[6-[(3aS,6aS)-6-oxo-2,3,3a,4,5,6a-hexahydropyrrolo[2,3-c]pyrrol-1-yl]benzimidazol-1-yl]-3-(1-hydroxyethyl)-2-pyridyl]-5-methyl-pyrazole-3-carbonitrile;
1-[2-[3-(difluoromethyl)-5-methyl-1,2,4-triazol-1-yl]-6-[5-[(6-methylpyridazin-3-yl)amino]benzimidazol-1-yl]-3-pyridyl]ethanol;
3-[[1-[6-[3-(difluoromethyl)-5-methyl-pyrazol-1-yl]-5-[(1˜{S})-1-hydroxyethyl]-2-pyridyl]-6-fluoro-benzimidazol-5-yl]amino]-˜{N},˜{N},6-trimethyl-pyridazine-4-carboxamide;
1-[2-[2-(difluoromethoxy)-5-methyl-4-pyridyl]-6-[6-fluoro-5-[(6-methylpyridazin-3-yl)amino]benzimidazol-1-yl]-3-pyridyl]ethanol;
1-[6-[6-fluoro-5-[(6-methylpyridazin-3-yl)amino]benzimidazol-1-yl]-3-(hydroxymethyl)-2-pyridyl]-5-methyl-pyrazole-3-carbonitrile;
3-[[1-[6-[3,5-bis(difluoromethyl)pyrazol-1-yl]-5-(hydroxymethyl)-2-pyridyl]benzimidazol-5-yl]amino]-N,N,6-trimethyl-pyridazine-4-carboxamide;
1-[6-[5-[(6-methylpyridazin-3-yl)amino]benzimidazol-1-yl]-2-[4-methyl-1-(2,2,2-trifluoroethyl)pyrazol-3-yl]-3-pyridyl]ethanol;
1-[2-[3-methyl-1-(2,2,2-trifluoroethyl)pyrazol-4-yl]-6-[5-[6-(morpholinomethyl)pyridazin-3-yl]oxybenzimidazol-1-yl]-3-pyridyl]ethanol;
1-[6-[5-(6-methylpyridazin-3-yl)oxybenzimidazol-1-yl]-2-[3-methyl-1-(2,2,2-trifluoroethyl)pyrazol-4-yl]-3-pyridyl]ethanol;
1-[6-[5-[6-[(4-methylpiperazin-1-yl)methyl]pyridazin-3-yl]oxybenzimidazol-1-yl]-2-[3-methyl-1-(2,2,2-trifluoroethyl)pyrazol-4-yl]-3-pyridyl]ethanol;
1-[2-[3-(difluoromethoxy)-5-methyl-pyrazol-1-yl]-6-[6-[(6-methylpyridazin-3-yl)amino]-5-(oxetan-3-yloxy)benzimidazol-1-yl]-3-pyridyl]ethanol;
1-[2-(3-chloro-5-methyl-pyrazol-1-yl)-6-[5-fluoro-6-[(6-methylpyridazin-3-yl)amino]benzimidazol-1-yl]-3-pyridyl]ethanol;
1-[2-(3-chloro-5-methyl-pyrazol-1-yl)-6-[6-fluoro-5-[(6-methylpyridazin-3-yl)amino]benzimidazol-1-yl]-3-pyridyl]ethanol;
1-[2-[1-(cyclopropylmethyl)-3-methyl-pyrazol-4-yl]-6-[6-fluoro-5-[(6-methylpyridazin-3-yl)amino]benzimidazol-1-yl]-3-pyridyl]ethanol;
1-[2-[3-(difluoromethoxy)-5-methyl-pyrazol-1-yl]-6-[5-[(6-methylpyridazin-3-yl)amino]-6-(oxetan-3-yloxy)benzimidazol-1-yl]-3-pyridyl]ethanol;
N-[1-[5-(1-hydroxyethyl)-6-[3-methyl-1-(2,2,2-trifluoroethyl)pyrazol-4-yl]-2-pyridyl]benzimidazol-5-yl]-2-(1-piperidyl)acetamide; 6-[3-(1-hydroxyethyl)-6-[5-[(6-methylpyridazin-3-yl)amino]benzimidazol-1-yl]-2-pyridyl]-6-azaspiro[3.4]octane-8-carbonitrile;
4-[3-(1-hydroxyethyl)-6-[5-[(6-methylpyridazin-3-yl)amino]benzimidazol-1-yl]-2-pyridyl]morpholine-2-carbonitrile;
N-[1-[6-[3-(difluoromethyl)-5-methyl-pyrazol-1-yl]-5-(1-hydroxyethyl)-2-pyridyl]-6-fluoro-benzimidazol-5-yl]cyclopropanecarboxamide;
1-[6-[6-(2,3-difluoro-4-methyl-anilino)-5-fluoro-benzimidazol-1-yl]-2-[3-(difluoromethyl)-5-methyl-pyrazol-1-yl]-3-pyridyl]ethanol;
1-[2-[1-(difluoromethyl)-4-methyl-pyrazol-3-yl]-6-[5-[(6-methylpyridazin-3-yl)amino]benzimidazol-1-yl]-3-pyridyl]ethanol;
1-[2-[3-(difluoromethyl)-5-methyl-pyrazol-1-yl]-6-[5-fluoro-6-(2,3,4-trifluoroanilino)benzimidazol-1-yl]-3-pyridyl]ethanol;
1-[3-(1-hydroxyethyl)-6-[6-(6-methyl-7-oxo-5H-pyrrolo[3,4-b]pyridin-2-yl)benzimidazol-1-yl]-2-pyridyl]-5-methyl-pyrazole-3-carbonitrile;
1-[3-(1-hydroxyethyl)-6-[5-(6-methyl-7-oxo-5H-pyrrolo[3,4-b]pyridin-2-yl)benzimidazol-1-yl]-2-pyridyl]-5-methyl-pyrazole-3-carbonitrile;
1-[2-[3-(difluoromethoxy)-5-methylpyrazol-1-yl]-6-[5-[[6-(difluoromethyl)pyridazin-3-yl]amino]benzimidazol-1-yl]pyridin-3-yl]ethanol;
1-[2-[3-(difluoromethyl)-5-ethyl-pyrazol-1-yl]-6-[6-[(6-methylpyridazin-3-yl)amino]benzimidazol-1-yl]-3-pyridyl]ethanol;
1-[2-[3-(difluoromethyl)-4,5-dimethyl-pyrazol-1-yl]-6-[5-[(6-methylpyridazin-3-yl)amino]benzimidazol-1-yl]-3-pyridyl]ethanol;
1-[2-[3-(difluoromethyl)-5-ethyl-pyrazol-1-yl]-6-[5-[(6-methylpyridazin-3-yl)amino]benzimidazol-1-yl]-3-pyridyl]ethanol;
1-[6-[6-[(6-chloropyridazin-3-yl)amino]-5-fluoro-benzimidazol-1-yl]-2-[3-(difluoromethyl)-5-methyl-pyrazol-1-yl]-3-pyridyl]ethanol;
1-[6-[5-[(6-chloropyridazin-3-yl)amino]-6-fluoro-benzimidazol-1-yl]-2-[3-(difluoromethyl)-5-methyl-pyrazol-1-yl]-3-pyridyl]ethanol;
(1S)-1-[2-[3-(difluoromethoxy)-5-methyl-pyrazol-1-yl]-6-[6-fluoro-5-(pyridazin-3-ylamino)benzimidazol-1-yl]-3-pyridyl]ethanol;
(1S)-1-[2-[3-(difluoromethoxy)-5-methyl-pyrazol-1-yl]-6-[6-fluoro-5-[(6-methylpyridazin-3-yl)amino]benzimidazol-1-yl]-3-pyridyl]ethanol;
(1R)-1-[2-[3-(difluoromethoxy)-5-methyl-pyrazol-1-yl]-6-[6-fluoro-5-[(6-methylpyridazin-3-yl)amino]benzimidazol-1-yl]-3-pyridyl]ethanol;
1-[6-[5-fluoro-6-[(5-methyl-1,3,4-oxadiazol-2-yl)amino]benzimidazol-1-yl]-3-(1-hydroxyethyl)-2-pyridyl]-5-methyl-pyrazole-3-carbonitrile;
1-[6-[6-fluoro-5-[(5-methyl-1,3,4-oxadiazol-2-yl)amino]benzimidazol-1-yl]-3-(1-hydroxyethyl)-2-pyridyl]-5-methyl-pyrazole-3-carbonitrile;
1-[6-[6-fluoro-5-(2,3,4-trifluoroanilino)benzimidazol-1-yl]-3-(1-hydroxyethyl)-2-pyridyl]-5-methyl-pyrazole-3-carbonitrile;
1-[6-[5-fluoro-6-(2,3,4-trifluoroanilino)benzimidazol-1-yl]-3-(1-hydroxyethyl)-2-pyridyl]-5-methyl-pyrazole-3-carbonitrile;
N-[1-[6-(3-cyano-5-methyl-pyrazol-1-yl)-5-(1-hydroxyethyl)-2-pyridyl]benzimidazol-5-yl]-1-fluoro-cyclopropanecarboxamide;
1-[6-[6-(6,7-dihydro-5˜{H}-cyclopenta[c]pyridazin-3-ylamino)-5-fluoro-benzimidazol-1-yl]-3-(1-hydroxyethyl)-2-pyridyl]-5-methyl-pyrazole-3-carbonitrile;
1-[6-[5-(6,7-dihydro-5˜{H}-cyclopenta[c]pyridazin-3-ylamino)-6-fluoro-benzimidazol-1-yl]-3-(1-hydroxyethyl)-2-pyridyl]-5-methyl-pyrazole-3-carbonitrile;
(1S)-1-[2-[3-(difluoromethoxy)-5-methyl-pyrazol-1-yl]-6-[5-[(6-methylpyridazin-3-yl)amino]-6-(oxetan-3-yloxy)benzimidazol-1-yl]-3-pyridyl]ethanol;
(1R)-1-[2-[3-(difluoromethoxy)-5-methyl-pyrazol-1-yl]-6-[5-[(6-methylpyridazin-3-yl)amino]-6-(oxetan-3-yloxy)benzimidazol-1-yl]-3-pyridyl]ethanol;
1-[6-[6-fluoro-5-[(5-methyl-1,3,4-thiadiazol-2-yl)amino]benzimidazol-1-yl]-3-(1-hydroxyethyl)-2-pyridyl]-5-methyl-pyrazole-3-carbonitrile;
3-fluoro-4-[3-(1-hydroxyethyl)-6-[5-[(6-methylpyridazin-3-yl)amino]benzimidazol-1-yl]-2-pyridyl]-1-(2,2,2-trifluoroethyl)pyridin-2-one;
1-[2-[3,5-bis(difluoromethyl)pyrazol-1-yl]-6-[6-fluoro-5-[(6-methylpyridazin-3-yl)amino]benzimidazol-1-yl]-3-pyridyl]ethanol;
1-[6-[6-(difluoromethoxy)-5-[(6-methylpyridazin-3-yl)amino]benzimidazol-1-yl]-3-(1-hydroxyethyl)-2-pyridyl]-5-methyl-pyrazole-3-carbonitrile;
1-[6-[6-fluoro-5-[(5-fluoro-6-methyl-3-pyridyl)amino]benzimidazol-1-yl]-3-(1-hydroxyethyl)-2-pyridyl]-5-methyl-pyrazole-3-carbonitrile;
1-[6-[5-[[5-(azetidin-1-yl)pyridazin-3-yl]amino]-6-fluoro-benzimidazol-1-yl]-2-[3-(difluoromethyl)-5-methyl-pyrazol-1-yl]-3-pyridyl]ethanol;
1-[6-[6-fluoro-5-[(5-fluoro-6-methyl-3-pyridyl)amino]benzimidazol-1-yl]-3-[(1S)-1-hydroxyethyl]-2-pyridyl]-5-methyl-pyrazole-3-carbonitrile;
1-[3-(1-hydroxyethyl)-6-[5-[2-(2-oxopyrrolidin-1-yl)oxazol-5-yl]benzimidazol-1-yl]-2-pyridyl]-5-methyl-pyrazole-3-carbonitrile;
1-[2-(4,4-difluoropyrrolidin-3-yl)oxy-6-[5-[(6-methylpyridazin-3-yl)amino]benzimidazol-1-yl]-3-pyridyl]ethanol;
1-[6-[5-(7,8-dihydro-5˜{H}-pyrano[4,3-c]pyridazin-3-ylamino)-6-methoxy-benzimidazol-1-yl]-3-(1-hydroxyethyl)-2-pyridyl]-5-methyl-pyrazole-3-carbonitrile;
1-[3-(1-hydroxyethyl)-6-[6-[2-(2-oxopyrrolidin-1-yl)oxazol-5-yl]benzimidazol-1-yl]-2-pyridyl]-5-methyl-pyrazole-3-carbonitrile;
1-[2-[6-(difluoromethoxy)-3-methyl-pyridazin-4-yl]-6-[5-[(6-methylpyridazin-3-yl)amino]benzimidazol-1-yl]-3-pyridyl]ethanol;
1-[2-[3-(difluoromethyl)-5-methoxy-pyrazol-1-yl]-6-[6-[(6-methylpyridazin-3-yl)amino]benzimidazol-1-yl]-3-pyridyl]ethanol;
1-[2-[3-(difluoromethyl)-5-methoxy-pyrazol-1-yl]-6-[5-[(6-methylpyridazin-3-yl)amino]benzimidazol-1-yl]-3-pyridyl]ethanol;
1-[2-[1-(2,3-difluoropropyl)-3-methyl-pyrazol-4-yl]-6-[5-[(6-methylpyridazin-3-yl)amino]benzimidazol-1-yl]-3-pyridyl]ethanol;
(1S)-1-[2-[3-(difluoromethoxy)-5-methyl-pyrazol-1-yl]-6-[6-methoxy-5-[(2-methylpyrimidin-5-yl)amino]benzimidazol-1-yl]-3-pyridyl]ethanol;
1-[6-[5-[(6-methylpyridazin-3-yl)amino]benzimidazol-1-yl]-2-[2-methyl-5-(2,2,2-trifluoroethyl)-1,2,4-triazol-3-yl]-3-pyridyl]ethanol;
1-[6-[6-(difluoromethoxy)-5-[(6-methylpyridazin-3-yl)amino]benzimidazol-1-yl]-3-[(1S)-1-hydroxyethyl]-2-pyridyl]-5-methyl-pyrazole-3-carbonitrile;
1-[6-[6-(difluoromethoxy)-5-[(6-methylpyridazin-3-yl)amino]benzimidazol-1-yl]-3-[(1R)-1-hydroxyethyl]-2-pyridyl]-5-methyl-pyrazole-3-carbonitrile;
1-[2-[3-(difluoromethoxy)-5-(difluoromethyl)pyrazol-1-yl]-6-[5-[(6-methylpyridazin-3-yl)amino]benzimidazol-1-yl]-3-pyridyl]ethanol;
1-[2-[3-(difluoromethoxy)-5-(difluoromethyl)pyrazol-1-yl]-6-[6-[(6-methylpyridazin-3-yl)amino]benzimidazol-1-yl]-3-pyridyl]ethanol;
1-[2-[3-(difluoromethoxy)-5-methyl-pyrazol-1-yl]-6-[6-[(6-methylpyridazin-3-yl)amino]-5-(oxetan-3-ylmethyl)benzimidazol-1-yl]-3-pyridyl]ethanol; and
1-[2-[3-(difluoromethoxy)-5-methyl-pyrazol-1-yl]-6-[5-[(6-methylpyridazin-3-yl)amino]-6-(oxetan-3-ylmethyl)benzimidazol-1-yl]-3-pyridyl]ethanol;
   or a pharmaceutically acceptable salt thereof.

70. A compound according to any one of embodiments 1 to 69, selected from
1-[2-[1-(2,2-difluoropropyl)-3-methyl-pyrazol-4-yl]-6-[5-[(6-methylpyridazin-3-yl)amino]benzimidazol-1-yl]-3-pyridyl]ethanol;
(1S)-1-[2-[3,5-bis(difluoromethyl)pyrazol-1-yl]-6-[5-[(6-methylpyridazin-3-yl)amino]benzimidazol-1-yl]-3-pyridyl]ethanol;
1-[2-[3-(difluoromethoxy)-5-methyl-pyrazol-1-yl]-6-[5-[(6-methylpyridazin-3-yl)amino]benzimidazol-1-yl]-3-pyridyl]ethanol;
(1S)-1-[2-[3-(difluoromethyl)-5-methyl-pyrazol-1-yl]-6-[6-fluoro-5-[(6-methylpyridazin-3-yl)amino]benzimidazol-1-yl]-3-pyridyl]ethanol;
1-[6-[6-fluoro-5-[(6-methylpyridazin-3-yl)amino]benzimidazol-1-yl]-2-[3-methyl-1-(2,2,2-trifluoroethyl)pyrazol-4-yl]-3-pyridyl]ethanol;
5-(difluoromethyl)-2-[3-(1-hydroxyethyl)-6-[5-[(6-methylpyridazin-3-yl)amino]benzimidazol-1-yl]-2-pyridyl]pyrazole-3-carbonitrile;
1-[6-[5-[(6-methylpyridazin-3-yl)amino]benzimidazol-1-yl]-2-[5-methyl-2-(2,2,2-trifluoroethyl)triazol-4-yl]-3-pyridyl]ethanol;
1-[3-(1-hydroxyethyl)-6-[5-[(6-methylpyridazin-3-yl)amino]benzimidazol-1-yl]-2-pyridyl]-4,5-dimethyl-pyrazole-3-carbonitrile;
1-[6-[5-[(6-methylpyridazin-3-yl)amino]benzimidazol-1-yl]-2-[5-methyl-1-(2,2,2-trifluoroethyl)triazol-4-yl]-3-pyridyl]ethanol;
1-[2-[1-(2,2-difluoroethyl)-3-methyl-pyrazol-4-yl]-6-[5-[(6-methylpyridazin-3-yl)amino]benzimidazol-1-yl]-3-pyridyl]ethanol;
1-[6-[5-[(6-methylpyridazin-3-yl)amino]benzimidazol-1-yl]-2-[4-methyl-1-(2,2,2-trifluoroethyl)pyrazol-3-yl]-3-pyridyl]ethanol;
1-[2-[1-(difluoromethyl)-4-methyl-pyrazol-3-yl]-6-[5-[(6-methylpyridazin-3-yl)amino]benzimidazol-1-yl]-3-pyridyl]ethanol;
(1S)-1-[2-[3-(difluoromethoxy)-5-methyl-pyrazol-yl]-6-[6-fluoro-5-(pyridazin-3-ylamino)benzimidazol-1-yl]-3-pyridyl]ethanol; and
(1S)-1-[2-[3-(difluoromethoxy)-5-methyl-pyrazol-1-yl]-6-[6-fluoro-5-[(6-methylpyridazin-3-yl)amino]benzimidazol-1-yl]-3-pyridyl]ethanol;
   or a pharmaceutically acceptable salt thereof.

70. A process for the preparation of a compound according to any one of embodiments 1 to 69, comprising one of the following steps:
(a) the reaction of a compound of formula (B1)

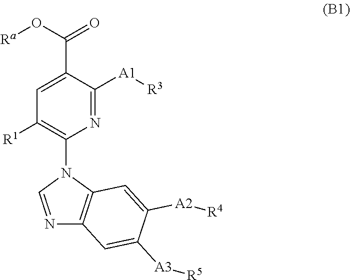

with a reducing agent;
(b) the reaction of a compound of formula (C1)

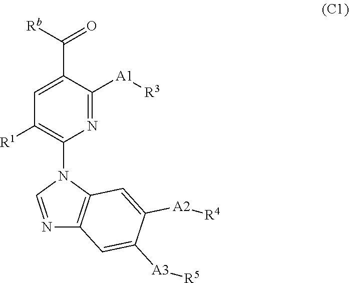

with a reducing agent; or
(c) the reaction of a compound (D1)

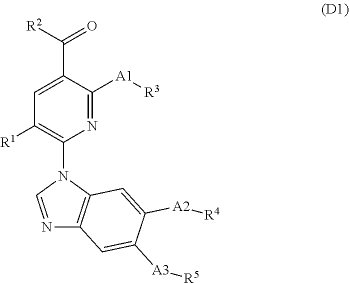

with a compound of formula R^{c}MgX,
   wherein A1, A2, A3, R¹, R², R³, R⁴ and R⁵ are as defined in any one of embodiments 1 to 65, R^{a} is alkyl or cycloalkyl, R^{b} is hydrogen or alkyl, R^{c} is alkyl or cycloalkyl, and X is halogen.

71. A compound according to any one of embodiments 1 to 69 when manufactured according to a process of embodiment 70.

72. A compound of formula (I) according to any one of embodiments 1 to 69 or a pharmaceutically acceptable salt thereof, for use as therapeutically active substance.

73. A pharmaceutical composition comprising a compound of formula (I) according to any one of embodiments 1 to 69 or a pharmaceutically acceptable salt thereof, and a therapeutically inert carrier.

74. The use of a compound of formula (I) according to any one of embodiments 1 to 69 or a pharmaceutically acceptable salt thereof, for the treatment or prophylaxis of rheumatoid arthritis, juvenile rheumatoid arthritis, non-alcoholic steatohepatitis (NASH), primary sclerosing cholangitis, giant cell vasculitis, inflammatory bowel diseases (IBD), atherosclerosis, type 2 diabetes or glomerulonephritis.

75. The use of a compound of formula (I) according to any one of embodiments 1 to 69 or a pharmaceutically acceptable salt thereof, for the preparation of a medicament for the treatment or prophylaxis rheumatoid arthritis, juvenile rheumatoid arthritis, non-alcoholic steatohepatitis (NASH), primary sclerosing cholangitis, giant cell vasculitis, inflammatory bowel diseases (IBD), atherosclerosis, type 2 diabetes or glomerulonephritis.

76. A compound of formula (I) according to any one of embodiments 1 to 69 or a pharmaceutically acceptable salt thereof, for use in the treatment or prophylaxis of rheumatoid arthritis, juvenile rheumatoid arthritis, non-alcoholic steatohepatitis (NASH), primary sclerosing cholangitis, giant cell vasculitis, inflammatory bowel diseases (IBD), atherosclerosis, type 2 diabetes or glomerulonephritis.

77. A method for the treatment or prophylaxis of rheumatoid arthritis, juvenile rheumatoid arthritis, non-alcoholic steatohepatitis (NASH), primary sclerosing cholangitis, giant cell vasculitis, inflammatory bowel diseases (IBD), atherosclerosis, type 2 diabetes or glomerulonephritis, which method comprises administering an effective amount of a compound of formula (I) according to any one of embodiments 1 to 69 or a pharmaceutically acceptable salt thereof, to a patient in need thereof.

1. A compound of formula (I) 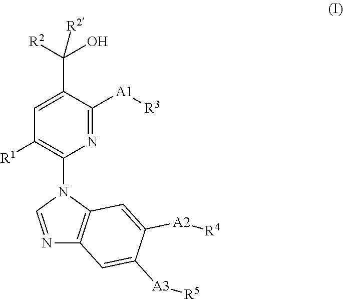
wherein
R¹ is hydrogen or halogen;
R² and R^{2′} are independently selected from hydrogen, alkyl, cyclopropyl, haloalkyl and alkoxyalkyl;
A1 is —O—, —NR⁶—, —(C═O)— or a bond;
R⁶ is hydrogen or alkyl;
R³ is hydroxyalkyl, heterocycloalkyl, heteroaryl, phenyl or cycloalkylalkyl, wherein heterocycloalkyl, heteroaryl, phenyl and cycloalkylalkyl are optionally substituted with 1, 2 or 3 substituents independently selected from R⁷; if A1 is —(C═O)—, then R³ can also be haloalkylamino;
each R⁷ is independently selected from alkoxy, alkylamino, alkyl, aminocarbonyl, amino, cyano, cycloalkylamino, haloalkyl, halocycloalkyl, halogen, heteroaryl, hydroxycarbonylamino, alkoxyalkyl, alkylaminocarbonyl, alkylsulfonyl, alkoxycarbonimidoyl, aminocarbonyl, hydroxy, cycloalkylalkyl, haloalkoxy, heterocycloalkyl and cycloalkyl;
A2 is —O—, —NH— or a bond;
R⁴ is hydrogen, alkyl, haloalkyl, alkoxyalkyl, dialkylaminoalkyl, cycloalkyl, cycloalkylcarbonyl, aryl, heteroaryl, heteroarylalkyl, heterocycloalkyl or heterocycloalkylalkyl; wherein aryl, cycloalkyl, heteroaryl, heteroarylalkyl, heterocycloalkyl and heterocycloalkylalkyl are optionally substituted with 1, 2 or 3 substituents independently selected from R⁸; if A2 is a bond, then R⁴ can also be halogen or cyano;
each R¹ is independently selected from alkyl, halogen, cyano, alkylsulfonyl, alkylaminocarbonyl, heterocycloalkyl and alkoxyheterocycloalkylalkyl;
A3 is —O—, —NR¹⁰— or a bond;
R⁵ is hydrogen, alkyl, alkylsulfonyl, cycloalkylcarbonyl, heterocycloalkylalkyl, heterocycloalkylalkylcarbonyl, aryl, heteroaryl or heterocycloalkyl, wherein cycloalkylcarbonyl, aryl, heteroaryl and heterocycloalkyl are optionally substituted with 1, 2 or 3 substituents independently selected from R⁹; if A3 is a bond, then R¹ can also be halogen or cyano;
each R⁹ is independently selected from alkoxy, halogen, dialkylaminocarbonyl, alkyl, alkoxyalkoxy, alkoxyheterocycloalkylalkyl, alkoxyheterocycloalkylcarbonyl, haloalkyl, haloalkoxy, heterocycloalkylalkoxy, heterocycloalkyl, heterocycloalkyloxy, hydroxy, hydroxyalkyl, alkylheterocycloalkyl, (haloalkyl)cycloalkyl, alkylheterocycloalkylalkyl, heterocycloalkylalkyl, alkylsulfonyl, (alkyl)heterocycloalkyl, alkylheterocycloalkyloxy, heterocycloalkylheterocycloalkyl, heterocycloalkylheterocycloalkyl, CH₃—O—(CH₂—CH₂—O)ₙ—, alkylaminocarbonyl and cyano; wherein n is selected from 5, 6, 7, 8 and 9; and
R¹⁰ is hydrogen or alkylcarbonyl;
or a pharmaceutically acceptable salt thereof.

2. A compound according to claim 1, wherein R¹ is hydrogen or chloro.

3. A compound according to claim 1, wherein R² and R^{2′} are independently selected from hydrogen, methyl, ethyl, cyclopropyl, difluoromethyl, trifluoromethyl and methoxymethyl.

4. A compound according to claim 1, wherein A1 is —O—, —NR⁶— or a bond; and wherein R⁶ is hydrogen or methyl.

5. A compound according to claim 1, wherein R³ is hydroxypentyl, hydroxybutyl, phenyl, cyclopropylmethyl, heterocycloalkyl or heteroaryl, and wherein phenyl, heterocycloalkyl and heteroaryl are optionally substituted with 1, 2 or 3 substituents independently selected from R⁷; if A1 is —(C═O)—, then R³ can also be trifluoroethylamino;
each R⁷ is independently selected from aminocarbonyl, amino, cyano, methoxy, ethoxy, chloro, fluoro, hydroxy, trifluoromethyl, trifluoroethyl, difluoromethyl, methylsulfonyl, methylaminocarbonyl, methoxyethyl, cyclopropylamino, difluoromethoxy, oxetan-3-yl, methylamino, 1H-pyrazol-4-yl, difluorocyclopropyl, methyl, ethyl, cyclopropylmethyl and cyclobutylmethyl.

6. A compound according to claim 1, wherein
A2 is —O—, —NH— or a bond;
R⁴ is hydrogen, haloalkyl, alkoxyalkyl, dialkylaminoalkyl, cycloalkyl, cycloalkylcarbonyl, aryl, heteroaryl, heteroarylalkyl, heterocycloalkyl or heterocycloalkylalkyl; wherein aryl, cycloalkyl, heteroaryl, heteroarylalkyl, heterocycloalkyl and heterocycloalkylalkyl are optionally substituted with 1, 2 or 3 substituents independently selected from R⁸; if A2 is a bond, then R⁴ can also be halogen or cyano; if A2 is —O— or —NH—, then R⁴ can also be alkyl;
each R⁸ is independently selected from alkyl, halogen, cyano, alkylsulfonyl, alkylaminocarbonyl, heterocycloalkyl and alkoxyheterocycloalkylalkyl;
A3 is —O—, —NH— or a bond;
R⁵ is hydrogen, alkylsulfonyl, cycloalkylcarbonyl, heterocycloalkylalkyl, heterocycloalkylalkylcarbonyl, aryl, heteroaryl or heterocycloalkyl, wherein aryl, cycloalkylcarbonyl, heteroaryl and heterocycloalkyl are optionally substituted with 1, 2 or 3 substituents independently selected from R⁹; if A3 is a bond, then R⁵ can also be halogen or cyano; if A3 is —O— or —NH—, then R⁵ can also be alkyl;
each R⁹ is independently selected from alkoxy, halogen, dialkylaminocarbonyl, alkyl, alkoxyalkoxy, alkoxyheterocycloalkylalkyl, alkoxyheterocycloalkylcarbonyl, haloalkyl, haloalkoxy, heterocycloalkylalkoxy, heterocycloalkyl, heterocycloalkyloxy, hydroxy, alkylheterocycloalkyl, alkylheterocycloalkylalkyl, heterocycloalkylalkyl, alkylsulfonyl, (alkyl)heterocycloalkyl, alkylheterocycloalkyloxy, heterocycloalkylheterocycloalkyl, heterocycloalkylheterocycloalkyl, CH₃—O—(CH₂—CH₂—O)ₙ—, alkylaminocarbonyl and cyano; wherein n is selected from 5, 6, 7, 8 and 9;
with the proviso that only one of R⁴ and R⁵ can be hydrogen.

7. A compound according to claim 1, wherein A2 is —O— or a bond.

8. A compound according to claim 1, wherein R⁴ is hydrogen, trifluoromethyl, methoxyethyl, dimethylaminoethyl, cyclopropylcarbonyl, morpholinoethyl, (1,1-dioxo-1,2-thiazolidin-2-yl)methyl, (2-oxopyrrolidin-1-yl)methyl, (2-oxo-1-piperidyl)methyl, heteroaryl or heterocycloalkyl, and wherein heteroaryl and heterocycloalkyl are optionally substituted with 1, 2 or 3 substituents independently selected from R⁸; if A2 is a bond, then R⁴ can also be fluoro, bromo or cyano; if A2 is —O— or —NH—, then R⁴ can also be methyl;
each R⁸ is independently selected from methyl, fluoro, cyano, methylsulfonyl, oxetan-3-yl and (3-methoxyazetidin-1-yl)methyl.

9. A compound according to claim 1, wherein A3 is —O— or —NH—.

10. A compound according to claim 1, wherein R⁵ is hydrogen, methylsulfonyl, morpholinoethyl, (2-oxopyrrolidin-1-yl)methyl, (2-oxo-1-piperidyl)methyl, (oxetan-3-yl)methyl, 1-piperidylethylcarbonyl, phenyl, heteroaryl or heterocycloalkyl; wherein phenyl, heteroaryl and heterocycloalkyl are optionally substituted with 1, 2 or 3 substituents independently selected from R⁹; if A3 is a bond, then R⁵ can also be fluoro or cyano; if A3 is —O— or —NH—, then R⁵ can also be methyl; and
wherein each R⁹ is independently selected from methyl, methoxy, oxetan-3-yl, azetidin-3-yl, azetidin-1-yl, 3,3-difluoroazetidin-1-yl, 2-oxopyrrolidin-1-yl, hydroxy, difluoromethyl, trifluoromethyl, (3-methoxyazetidin-1-yl)methyl, 3-methoxyazetidine-1-carbonyl, methoxyethoxy, difluoroethoxy, difluoromethoxy, [2-(1,1-dioxo-1,4-thiazinan-4-yl)ethoxy], oxetan-3-yloxy, chloro, dimethylaminocarbonyl, cyano, methylsulfonyl, morpholinomethyl, morpholino, (4-methylpiperazin-1-yl)methyl, CH₃—O—(CH₂—CH₂—O)r-, oxetan-3-yl, [1-(oxetan-3-yl)pyrrolidin-2-yl] and (2-methyl-1,3-dioxolan-2-yl).

11. A compound according to claim 1, selected from
[2-(3-chlorophenyl)-6-(5,6-dimethoxybenzimidazol-1-yl)-3-pyridyl]methanol;
(2-(3-chloro-2-fluorophenyl)-6-(5,6-dimethoxy-1H-benzo[d]imidazol-1-yl)pyridin-3-yl)methanol;
(6-(5,6-dimethoxy-1H-benzo[d]imidazol-1-yl)-2-(4-fluoro-2-methoxyphenyl)pyridin-3-yl)methanol;
1-(6-(5,6-dimethoxy-1H-benzo[d]imidazol-1-yl)-2-(4-fluoro-2-methoxyphenyl)pyridin-3-yl)ethan-1-ol;
1-(6-(5,6-dimethoxy-1H-benzo[d]imidazol-1-yl)-2-(4-fluoro-2-methoxyphenyl)pyridin-3-yl)propan-1-ol;
1-(2-(3-chlorophenyl)-6-(5,6-dimethoxy-1H-benzo[d]imidazol-1-yl)pyridin-3-yl)ethan-1-ol;
1-(2-(3-chloro-2-fluorophenyl)-6-(5,6-dimethoxy-1H-benzo[d]imidazol-1-yl)pyridin-3-yl)ethan-1-ol;
(S)-1-(6-(5,6-dimethoxy-1H-benzo[d]imidazol-1-yl)-2-(4-fluoro-2-methoxyphenyl)pyridin-3-yl)ethan-1-ol;
(R)-1-(6-(5,6-dimethoxy-1H-benzo[d]imidazol-1-yl)-2-(4-fluoro-2-methoxyphenyl)pyridin-3-yl)ethan-1-ol;
cyclopropyl(6-(5,6-dimethoxy-1H-benzo[d]imidazol-1-yl)-2-(4-fluoro-2-methoxyphenyl)pyridin-3-yl)methanol;
1-(2-(3-chlorophenyl)-6-(5,6-dimethoxy-1H-benzo[d]imidazol-1-yl)pyridin-3-yl)propan-1-ol;
(2-(3-chlorophenyl)-6-(5,6-dimethoxy-1H-benzo[d]imidazol-1-yl)pyridin-3-yl)(cyclopropyl)methanol;
(6-(5,6-dimethoxy-1H-benzo[d]imidazol-1-yl)-2-morpholinopyridin-3-yl)methanol;
(6-(5,6-dimethoxy-1H-benzo[d]imidazol-1-yl)-2-(pyrrolidin-1-yl)pyridin-3-yl)methanol;
(6-(5,6-dimethoxy-1H-benzo[d]imidazol-1-yl)-2-(piperidin-1-yl)pyridin-3-yl)methanol;
5-((6-(5,6-dimethoxy-1H-benzo[d]imidazol-1-yl)-3-(hydroxymethyl)pyridin-2-yl)amino)pentan-1-ol;
1-(6-(5,6-dimethoxy-1H-benzo[d]imidazol-1-yl)-3-(hydroxymethyl)pyridin-2-yl)pyrrolidin-2-one;
4-((6-(5,6-dimethoxy-1H-benzo[d]imidazol-1-yl)-3-(hydroxymethyl)pyridin-2-yl)amino)butan-1-ol;
(S)-2-(6-(5,6-dimethoxy-1H-benzo[d]imidazol-1-yl)-3-(1-hydroxyethyl)pyridin-2-yl)benzamide;
(R)-2-(6-(5,6-dimethoxy-1H-benzo[d]imidazol-1-yl)-3-(1-hydroxyethyl)pyridin-2-yl)benzamide;
(S)-3-(6-(5,6-dimethoxy-1H-benzo[d]imidazol-1-yl)-3-(1-hydroxyethyl)pyridin-2-yl)benzonitrile;
(R)-3-(6-(5,6-dimethoxy-1H-benzo[d]imidazol-1-yl)-3-(1-hydroxyethyl)pyridin-2-yl)benzonitrile;
(S)-4-(6-(5,6-dimethoxy-1H-benzo[d]imidazol-1-yl)-3-(1-hydroxyethyl)pyridin-2-yl)benzonitrile;
(R)-4-(6-(5,6-dimethoxy-1H-benzo[d]imidazol-1-yl)-3-(1-hydroxyethyl)pyridin-2-yl)benzonitrile;
(S)-6-(5,6-dimethoxy-1H-benzo[d]imidazol-1-yl)-3-(1-hydroxyethyl)-1′-methyl-[2,4′-bipyridin]-2′(1′H)-one;
(R)-6-(5,6-dimethoxy-1H-benzo[d]imidazol-1-yl)-3-(1-hydroxyethyl)-1′-methyl-[2,4′-bipyridin]-2′(1′H)-one;
1-[6-[5-(2-morpholinoethoxy)benzimidazol-1-yl]-2-phenoxy-3-pyridyl]ethanol;
1-[2-anilino-6-[5-(2-morpholinoethoxy)benzimidazol-1-yl]-3-pyridyl]ethanol;
1-[6-[5-(2-morpholinoethoxy)benzimidazol-1-yl]-2-[3-(trifluoromethyl)pyrazol-1-yl]-3-pyridyl]ethanol;
1-[6-(5,6-dimethoxybenzimidazol-1-yl)-2-[3-(trifluoromethyl)pyrazol-1-yl]-3-pyridyl]ethanol;
1-[3-(1-hydroxyethyl)-6-[5-[(6-methylpyridazin-3-yl)amino]benzimidazol-1-yl]-2-pyridyl]-5-methyl-pyrazole-3-carbonitrile;
1-[3-(1-hydroxyethyl)-6-[5-(4-methylpiperazin-1-yl)benzimidazol-1-yl]-2-pyridyl]-5-methyl-pyrazole-3-carbonitrile;
1-[3-(1-hydroxyethyl)-6-[5-[4-(oxetan-3-yl)piperazin-1-yl]benzimidazol-1-yl]-2-pyridyl]-5-methyl-pyrazole-3-carbonitrile;
1-[3-(1-hydroxyethyl)-6-[5-(2-morpholinoethoxy)benzimidazol-1-yl]-2-pyridyl]-5-methyl-pyrazole-3-carbonitrile;
1-[3-(1-hydroxyethyl)-6-[5-[[1-(oxetan-3-yl)pyrazol-4-yl]amino]benzimidazol-1-yl]-2-pyridyl]-5-methyl-pyrazole-3-carbonitrile;
1-[6-[5-[[1-(azetidin-3-yl)pyrazol-4-yl]amino]benzimidazol-1-yl]-3-(1-hydroxyethyl)-2-pyridyl]-5-methyl-pyrazole-3-carbonitrile;
1-[3-(1-hydroxyethyl)-6-[6-(6-methylpyridazin-3-yl)oxybenzimidazol-1-yl]-2-pyridyl]-5-methyl-pyrazole-3-carbonitrile;
1-[3-(1-hydroxyethyl)-6-[5-(6-methylpyridazin-3-yl)oxybenzimidazol-1-yl]-2-pyridyl]-5-methyl-pyrazole-3-carbonitrile;
5-[3-(1-hydroxyethyl)-6-[5-[(6-methylpyridazin-3-yl)amino]benzimidazol-1-yl]-2-pyridyl]-1-methyl-pyrazole-4-carbonitrile;
1-[3-(1-hydroxyethyl)-6-[5-[rac-(3R)-3-hydroxypyrrolidin-1-yl]benzimidazol-1-yl]-2-pyridyl]-5-methyl-pyrazole-3-carbonitrile;
1-[3-(1-hydroxyethyl)-6-[5-[(3S)-3-hydroxypyrrolidin-1-yl]benzimidazol-1-yl]-2-pyridyl]-5-methyl-pyrazole-3-carbonitrile;
1-[3-(1-hydroxyethyl)-6-[5-[(6-methyl-3-pyridyl)amino]benzimidazol-1-yl]-2-pyridyl]-5-methyl-pyrazole-3-carbonitrile;
1-[3-(1-hydroxyethyl)-6-[5-[(5-methylpyrazin-2-yl)amino]benzimidazol-1-yl]-2-pyridyl]-5-methyl-pyrazole-3-carbonitrile;
1-[3-(1-hydroxyethyl)-6-[5-[(5-methylpyrimidin-2-yl)amino]benzimidazol-1-yl]-2-pyridyl]-5-methyl-pyrazole-3-carbonitrile;
1-[3-(1-hydroxyethyl)-6-[5-[(5-methyl-2-pyridyl)amino]benzimidazol-1-yl]-2-pyridyl]-5-methyl-pyrazole-3-carbonitrile;
1-[2-(1,1-dioxo-1,2-thiazolidin-2-yl)-6-[5-[(6-methylpyridazin-3-yl)amino]benzimidazol-1-yl]-3-pyridyl]ethanol;
1-[3-(1-hydroxyethyl)-6-[5-[(2-methylpyrimidin-5-yl)amino]benzimidazol-1-yl]-2-pyridyl]-5-methyl-pyrazole-3-carbonitrile;
1-[3-(1-hydroxyethyl)-6-[5-(pyridazin-4-ylamino)benzimidazol-1-yl]-2-pyridyl]-5-methyl-pyrazole-3-carbonitrile;
1-[3-(1-hydroxyethyl)-6-[5-[(6-methoxypyridazin-3-yl)amino]benzimidazol-1-yl]-2-pyridyl]-5-methyl-pyrazole-3-carbonitrile;
1-[3-(1-hydroxyethyl)-6-[5-(pyridazin-3-ylamino)benzimidazol-1-yl]-2-pyridyl]-5-methyl-pyrazole-3-carbonitrile;
1-[6-[5-[[6-(difluoromethyl)pyridazin-3-yl]amino]benzimidazol-1-yl]-3-(1-hydroxyethyl)-2-pyridyl]-5-methyl-pyrazole-3-carbonitrile;
1-[6-[6-fluoro-5-[(6-methylpyridazin-3-yl)amino]benzimidazol-1-yl]-3-(1-hydroxyethyl)-2-pyridyl]-5-methyl-pyrazole-3-carbonitrile;
1-[6-[5-fluoro-6-[(6-methylpyridazin-3-yl)amino]benzimidazol-1-yl]-3-(1-hydroxyethyl)-2-pyridyl]-5-methyl-pyrazole-3-carbonitrile;
1-[6-[5-[(6-methylpyridazin-3-yl)amino]benzimidazol-1-yl]-2-[3-(trifluoromethyl)-4,5,6,7-tetrahydropyrazolo[4,3-c]pyridin-1-yl]-3-pyridyl]ethanol;
1-[6-[6-[(6-methylpyridazin-3-yl)amino]benzimidazol-1-yl]-2-[3-(trifluoromethyl)-4,5,6,7-tetrahydropyrazolo[4,3-c]pyridin-1-yl]-3-pyridyl]ethanol;
1-[3-(1-hydroxyethyl)-6-[5-[(5-methyl-1,3,4-oxadiazol-2-yl)amino]benzimidazol-1-yl]-2-pyridyl]-5-methyl-pyrazole-3-carbonitrile;
2-[3-(1-hydroxyethyl)-6-[5-[(6-methylpyridazin-3-yl)amino]benzimidazol-1-yl]-2-pyridyl]-5-methyl-pyrazole-3-carbonitrile;
(3R)-1-[3-(1-hydroxyethyl)-6-[6-[(6-methylpyridazin-3-yl)amino]benzimidazol-1-yl]-2-pyridyl]pyrrolidine-3-carbonitrile;
rac-(3R)-1-[3-(1-hydroxyethyl)-6-[5-[(6-methylpyridazin-3-yl)amino]benzimidazol-1-yl]-2-pyridyl]pyrrolidine-3-carbonitrile;
1-[2-(3,5-dimethylpyrazol-1-yl)-6-[6-[(6-methylpyridazin-3-yl)amino]benzimidazol-1-yl]-3-pyridyl]ethanol;
1-[2-(3,5-dimethylpyrazol-1-yl)-6-[5-[(6-methylpyridazin-3-yl)amino]benzimidazol-1-yl]-3-pyridyl]ethanol;
5-methyl-1-[6-[5-[(6-methylpyridazin-3-yl)amino]benzimidazol-1-yl]-3-[rac-(1R)-1-hydroxyethyl]-2-pyridyl]pyrazole-3-carbonitrile;
5-methyl-1-[6-[5-[(6-methylpyridazin-3-yl)amino]benzimidazol-1-yl]-3-[rac-(1S)-1-hydroxyethyl]-2-pyridyl]pyrazole-3-carbonitrile;
1-[3-(1-hydroxyethyl)-6-[5-[(5-methyl-1,3,4-thiadiazol-2-yl)amino]benzimidazol-1-yl]-2-pyridyl]-5-methyl-pyrazole-3-carbonitrile;
1-[3-(1-hydroxyethyl)-6-[5-[[1-(oxetan-3-yl)-4-piperidyl]oxy]benzimidazol-1-yl]-2-pyridyl]-5-methyl-pyrazole-3-carbonitrile;
1-[3-(1-hydroxyethyl)-6-[6-[[1-(oxetan-3-yl)-4-piperidyl]oxy]benzimidazol-1-yl]-2-pyridyl]-5-methyl-pyrazole-3-carbonitrile;
1-[3-(1-hydroxyethyl)-6-[5-[(6-methylpyridazin-3-yl)amino]benzimidazol-1-yl]-2-pyridyl]pyrazole-3-carbonitrile;
1-[[[3-(1-hydroxyethyl)-6-[5-[(6-methylpyridazin-3-yl)amino]benzimidazol-1-yl]-2-pyridyl]amino]methyl]cyclopropanecarbonitrile;
1-[[[3-(1-hydroxyethyl)-6-[6-[(6-methylpyridazin-3-yl)amino]benzimidazol-1-yl]-2-pyridyl]amino]methyl]cyclopropanecarbonitrile;
5-[3-(1-hydroxyethyl)-6-[5-[(6-methylpyridazin-3-yl)amino]benzimidazol-1-yl]-2-pyridyl]pyridine-3-carbonitrile;
1-[3-(1-hydroxyethyl)-6-[5-(1,2,4-triazin-3-ylamino)benzimidazol-1-yl]-2-pyridyl]-5-methyl-pyrazole-3-carbonitrile;
1-[3-(1-hydroxyethyl)-6-[6-[(6-methylpyridazin-3-yl)amino]benzimidazol-1-yl]-2-pyridyl]-3-methyl-pyrrolidine-3-carbonitrile;
1-[3-(1-hydroxyethyl)-6-[5-[(6-methylpyridazin-3-yl)amino]benzimidazol-1-yl]-2-pyridyl]-3-methyl-pyrrolidine-3-carbonitrile;
1-[6-[5-[(6-methylpyridazin-3-yl)amino]benzimidazol-1-yl]-2-[2-methyl-5-(trifluoromethyl)pyrazol-3-yl]-3-pyridyl]ethanol;
1-[3-(1-hydroxyethyl)-6-[5-(isoxazol-3-ylamino)benzimidazol-1-yl]-2-pyridyl]-5-methyl-pyrazole-3-carbonitrile;
3-(1-hydroxyethyl)-6-[5-[(6-methylpyridazin-3-yl)amino]benzimidazol-1-yl]-N-(2,2,2-trifluoroethyl)pyridine-2-carboxamide;
1-[6-[6-[(6-methylpyridazin-3-yl)amino]benzimidazol-1-yl]-2-[rac-(3aR,6aS)-2,3,3a,5,6,6a-hexahydro-1H-pyrrolo[3,2-b]pyrrol-4-yl]-3-pyridyl]ethanol;
1-[6-[6-[(6-methylpyridazin-3-yl)amino]benzimidazol-1-yl]-2-[rac-(3aS,6aR)-2,3,3a,5,6,6a-hexahydro-1H-pyrrolo[3,2-b]pyrrol-4-yl]-3-pyridyl]ethanol;
1-[6-[5-[(6-methylpyridazin-3-yl)amino]benzimidazol-1-yl]-2-[rac-(3aR,6aS)-2,3,3a,5,6,6a-hexahydro-1H-pyrrolo[3,2-b]pyrrol-4-yl]-3-pyridyl]ethanol;
1-[2-[3-(difluoromethyl)-5-methyl-pyrazol-1-yl]-6-[6-[(6-methylpyridazin-3-yl)amino]benzimidazol-1-yl]-3-pyridyl]ethanol;
1-[2-[3-(difluoromethyl)-5-methyl-pyrazol-1-yl]-6-[5-[(6-methylpyridazin-3-yl)amino]benzimidazol-1-yl]-3-pyridyl]ethanol;
1-[6-[6-bromo-5-[4-(oxetan-3-yl)piperazin-1-yl]benzimidazol-1-yl]-3-(1-hydroxyethyl)-2-pyridyl]-5-methyl-pyrazole-3-carbonitrile;
1-[2-(3-ethoxy-5-methyl-pyrazol-1-yl)-6-[5-[(6-methylpyridazin-3-yl)amino]benzimidazol-1-yl]-3-pyridyl]ethanol;
1-[2-(3-ethoxy-5-methyl-pyrazol-1-yl)-6-[6-[(6-methylpyridazin-3-yl)amino]benzimidazol-1-yl]-3-pyridyl]ethanol;
1-[2-(6-methylpyridazin-4-yl)-6-[5-[(6-methylpyridazin-3-yl)amino]benzimidazol-1-yl]-3-pyridyl]ethanol;
4-[3-(1-hydroxyethyl)-6-[5-[(6-methylpyridazin-3-yl)amino]benzimidazol-1-yl]-2-pyridyl]-1-(2,2,2-trifluoroethyl)piperazin-2-one;
4-[3-(1-hydroxyethyl)-6-[6-[(6-methylpyridazin-3-yl)amino]benzimidazol-1-yl]-2-pyridyl]-1-(2,2,2-trifluoroethyl)piperazin-2-one;
1-[[3-(1-hydroxyethyl)-6-[6-[(6-methylpyridazin-3-yl)amino]benzimidazol-1-yl]-2-pyridyl]oxymethyl]cyclopropanecarbonitrile;
1-[[3-(1-hydroxyethyl)-6-[5-[(6-methylpyridazin-3-yl)amino]benzimidazol-1-yl]-2-pyridyl]oxymethyl]cyclopropanecarbonitrile;
1-[3-(1-hydroxyethyl)-6-[5-methoxy-6-[(6-methylpyridazin-3-yl)amino]benzimidazol-1-yl]-2-pyridyl]-5-methyl-pyrazole-3-carbonitrile;
1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanol;
1-[3-(1-hydroxyethyl)-6-[6-[(6-methylpyridazin-3-yl)amino]benzimidazol-1-yl]-2-pyridyl]-N,5-dimethyl-pyrazole-3-carboxamide;
1-[2-(5-methyl-3-methylsulfonyl-pyrazol-1-yl)-6-[5-[(6-methylpyridazin-3-yl)amino]benzimidazol-1-yl]-3-pyridyl]ethanol;
1-[3-(1-hydroxyethyl)-6-[5-[(2-keto-1-methyl-pyrimidin-4-yl)amino]benzimidazol-1-yl]-2-pyridyl]-5-methyl-pyrazole-3-carbonitrile;
5-methyl-1-[6-[5-[(6-methylpyridazin-3-yl)amino]benzimidazol-1-yl]-3-(2,2,2-trifluoro-1-hydroxy-ethyl)-2-pyridyl]pyrazole-3-carbonitrile;
1-[2-[1-(difluoromethyl)-3-methyl-pyrazol-4-yl]-6-[5-[(6-methylpyridazin-3-yl)amino]benzimidazol-1-yl]-3-pyridyl]ethanol;
1-[3-(1-hydroxyethyl)-6-[5-[(6-methylpyridazin-3-yl)amino]benzimidazol-1-yl]-2-pyridyl]-5-methyl-pyrazole-4-carbonitrile;
1-[3-(1-hydroxyethyl)-6-[6-[(6-methylpyridazin-3-yl)amino]benzimidazol-1-yl]-2-pyridyl]-5-methyl-pyrazole-4-carbonitrile;
1-[3-(1-hydroxyethyl)-6-[5-[(6-methylpyridazin-3-yl)amino]benzimidazol-1-yl]-2-pyridyl]-N,5-dimethyl-pyrazole-3-carboxamide;
1-[3-(1-hydroxyethyl)-6-[5-[(2-keto-1-methyl-4-piperidyl)amino]benzimidazol-1-yl]-2-pyridyl]-5-methyl-pyrazole-3-carbonitrile;
[2-(3-methoxy-5-methyl-pyrazol-1-yl)-6-[5-[(6-methylpyridazin-3-yl)amino]benzimidazol-1-yl]-3-pyridyl]methanol;
1-[2-(3,5-dimethylisoxazol-4-yl)-6-[5-[(6-methylpyridazin-3-yl)amino]benzimidazol-1-yl]-3-pyridyl]ethanol;
1-[3-(1-hydroxyethyl)-6-[5-[(4-methoxypyridazin-3-yl)amino]benzimidazol-1-yl]-2-pyridyl]-5-methyl-pyrazole-3-carbonitrile;
1-[3-(1-hydroxyethyl)-6-[5-[(5-keto-1-methyl-pyrrolidin-3-yl)amino]benzimidazol-1-yl]-2-pyridyl]-5-methyl-pyrazole-3-carbonitrile;
1-[6-[5-[(6-methylpyridazin-3-yl)amino]benzimidazol-1-yl]-2-(3-methyl-4-pyridyl)-3-pyridyl]ethanol;
1-[2-[4-(cyclopropylamino)pyrimidin-5-yl]-6-[5-[(6-methylpyridazin-3-yl)amino]benzimidazol-1-yl]-3-pyridyl]ethanol;
5-[3-(1-hydroxyethyl)-6-[5-[(6-methylpyridazin-3-yl)amino]benzimidazol-1-yl]-2-pyridyl]-4-(trifluoromethyl)pyridin-2-ol;
3-[3-(1-hydroxyethyl)-6-[5-[(6-methylpyridazin-3-yl)amino]benzimidazol-1-yl]-2-pyridyl]-6,7-dihydro-5H-pyrazolo[1,5-a]pyrazin-4-one;
1-[6-[6-fluoro-5-[[(3S,4R)-4-fluoropyrrolidin-3-yl]amino]benzimidazol-1-yl]-3-(1-hydroxyethyl)-2-pyridyl]-5-methyl-pyrazole-3-carbonitrile;
1-[2-[2-(difluoromethoxy)-5-methyl-4-pyridyl]-6-[5-[(6-methylpyridazin-3-yl)amino]benzimidazol-1-yl]-3-pyridyl]ethanol;
1-[2-(2-fluoro-5-methyl-4-pyridyl)-6-[5-[(6-methylpyridazin-3-yl)amino]benzimidazol-1-yl]-3-pyridyl]ethanol;
1-[3-(1-hydroxyethyl)-6-[6-[(6-methylpyridazin-3-yl)amino]benzimidazol-1-yl]-2-pyridyl]-4-methyl-pyrazole-3-carbonitrile;
1-[3-(1-hydroxyethyl)-6-[5-[(6-methylpyridazin-3-yl)amino]benzimidazol-1-yl]-2-pyridyl]-4-methyl-pyrazole-3-carbonitrile;
1-[6-[5-[(6-methylpyridazin-3-yl)amino]benzimidazol-1-yl]-2-[5-methyl-3-(trifluoromethyl)-6,7-dihydro-4H-pyrazolo[4,3-c]pyridin-1-yl]-3-pyridyl]ethanol;
1-[[[3-(1-hydroxyethyl)-6-[6-[(6-methylpyridazin-3-yl)amino]benzimidazol-1-yl]-2-pyridyl]-methyl-amino]methyl]cyclopropanecarbonitrile;
1-[[[3-(1-hydroxyethyl)-6-[5-[(6-methylpyridazin-3-yl)amino]benzimidazol-1-yl]-2-pyridyl]-methyl-amino]methyl]cyclopropanecarbonitrile;
1-[2-(2-chloro-5-fluoro-3-pyridyl)-6-[5-[(6-methylpyridazin-3-yl)amino]benzimidazol-1-yl]-3-pyridyl]ethanol;
1-[3-(1-hydroxyethyl)-6-[6-[[6-[(3-methoxyazetidin-1-yl)methyl]pyridazin-3-yl]amino]benzimidazol-1-yl]-2-pyridyl]-5-methyl-pyrazole-3-carbonitrile;
1-[3-(1-hydroxyethyl)-6-[5-[[6-[(3-methoxyazetidin-1-yl)methyl]pyridazin-3-yl]amino]benzimidazol-1-yl]-2-pyridyl]-5-methyl-pyrazole-3-carbonitrile;
1-[6-[5-[(6-methylpyridazin-3-yl)amino]benzimidazol-1-yl]-2-[5-(oxetan-3-yl)-3-(trifluoromethyl)-6,7-dihydro-4H-pyrazolo[4,3-c]pyridin-1-yl]-3-pyridyl]ethanol;
1-[3-(1-hydroxyethyl)-6-[6-[(6-methylpyridazin-3-yl)amino]benzimidazol-1-yl]-2-pyridyl]-3-methyl-pyrazole-4-carbonitrile;
1-[2-[2-(methylamino)-3-pyridyl]-6-[5-[(6-methylpyridazin-3-yl)amino]benzimidazol-1-yl]-3-pyridyl]ethanol;
1-[3-(1-hydroxyethyl)-6-[5-[(6-methylpyridazin-3-yl)amino]benzimidazol-1-yl]-2-pyridyl]-3-methyl-pyrazole-4-carbonitrile;
1-[3-(1-hydroxyethyl)-6-[5-[(6-methylpyridazin-3-yl)amino]benzimidazol-1-yl]-2-pyridyl]-3-methyl-azetidine-3-carbonitrile;
1-[3-(1-hydroxyethyl)-6-[6-[(6-methylpyridazin-3-yl)amino]benzimidazol-1-yl]-2-pyridyl]-3-methyl-azetidine-3-carbonitrile;
1-[3-(1-hydroxyethyl)-6-[6-methoxy-5-[(6-methylpyridazin-3-yl)amino]benzimidazol-1-yl]-2-pyridyl]-5-methyl-pyrazole-3-carbonitrile;
1-[6-[5-[(6-methylpyridazin-3-yl)amino]benzimidazol-1-yl]-2-[2-(1H-pyrazol-4-yl)-3-pyridyl]-3-pyridyl]ethanol;
1-[3-(1-hydroxyethyl)-6-[6-(2-methoxyethoxy)-5-[(6-methylpyridazin-3-yl)amino]benzimidazol-1-yl]-2-pyridyl]-5-methyl-pyrazole-3-carbonitrile;
1-[6-[5-fluoro-6-[[rac-(3R,4S)-4-fluoropyrrolidin-3-yl]amino]benzimidazol-1-yl]-3-(1-hydroxyethyl)pyridin-2-yl]-5-methylpyrazole-3-carbonitrile;
1-[6-[6-[2-(dimethylamino)ethoxy]-5-[(6-methylpyridazin-3-yl)amino]benzimidazol-1-yl]-3-(1-hydroxyethyl)-2-pyridyl]-5-methyl-pyrazole-3-carbonitrile;
1-[6-[5-bromo-6-[4-(oxetan-3-yl)piperazino]benzimidazol-1-yl]-3-(1-hydroxyethyl)-2-pyridyl]-5-methyl-pyrazole-3-carbonitrile;
1-[3-(1-hydroxyethyl)-6-[5-[[6-(3-methoxyazetidine-1-carbonyl)pyridazin-3-yl]amino]benzimidazol-1-yl]-2-pyridyl]-5-methyl-pyrazole-3-carbonitrile;
1-[3-(1-hydroxyethyl)-6-[5-[(6-methylpyridazin-3-yl)amino]-6-pyrrolidin-3-yloxy-benzimidazol-1-yl]-2-pyridyl]-5-methyl-pyrazole-3-carbonitrile;
1-[3-(1-hydroxyethyl)-6-[5-[[6-(2-methoxyethoxy)pyridazin-3-yl]amino]benzimidazol-1-yl]-2-pyridyl]-5-methyl-pyrazole-3-carbonitrile;
1-[2-(1H-benzotriazol-4-yl)-6-[5-[(6-methylpyridazin-3-yl)amino]benzimidazol-1-yl]-3-pyridyl]ethanol;
1-[6-[5-[[6-(2,2-difluoroethoxy)pyridazin-3-yl]amino]benzimidazol-1-yl]-3-(1-hydroxyethyl)-2-pyridyl]-5-methyl-pyrazole-3-carbonitrile;
1-[6-[5-[[6-(difluoromethoxy)pyridazin-3-yl]amino]benzimidazol-1-yl]-3-(1-hydroxyethyl)-2-pyridyl]-5-methyl-pyrazole-3-carbonitrile;
1-[3-(2,2-difluoro-1-hydroxy-ethyl)-6-[5-[(6-methylpyridazin-3-yl)amino]benzimidazol-1-yl]-2-pyridyl]-5-methyl-pyrazole-3-carbonitrile;
1-[3-(2,2-difluoro-1-hydroxy-ethyl)-6-[6-[(6-methylpyridazin-3-yl)amino]benzimidazol-1-yl]-2-pyridyl]-5-methyl-pyrazole-3-carbonitrile;
1-[6-[6-[(1,1-dioxo-1,2-thiazolidin-2-yl)methyl]benzimidazol-1-yl]-3-(1-hydroxyethyl)-2-pyridyl]-5-methyl-pyrazole-3-carbonitrile;
1-[6-[5-[(1,1-dioxo-1,2-thiazolidin-2-yl)methyl]benzimidazol-1-yl]-3-(1-hydroxyethyl)-2-pyridyl]-5-methyl-pyrazole-3-carbonitrile;
3-[3-(1-hydroxyethyl)-6-[5-[(6-methylpyridazin-3-yl)amino]benzimidazol-1-yl]-2-pyridyl]-2-methoxy-phenol;
3-[3-(1-hydroxyethyl)-6-[5-[(6-methylpyridazin-3-yl)amino]benzimidazol-1-yl]-2-pyridyl]furan-2-carbonitrile;
1-[3-(1-hydroxyethyl)-6-[5-[(6-methylpyridazin-3-yl)amino]benzimidazol-1-yl]pyridin-2-yl]-4,5,6,7-tetrahydropyrazolo[4,3-c]pyridine-3-carbonitrile;
1-[6-[5-[[6-[2-(1,1-diketo-1,4-thiazinan-4-yl)ethoxy]pyridazin-3-yl]amino]benzimidazol-1-yl]-3-(1-hydroxyethyl)-2-pyridyl]-5-methyl-pyrazole-3-carbonitrile;
1-[3-(1-hydroxyethyl)-6-[5-[[6-(oxetan-3-yloxy)pyridazin-3-yl]amino]benzimidazol-1-yl]-2-pyridyl]-5-methyl-pyrazole-3-carbonitrile;
1-[6-[5-[(6-methylpyridazin-3-yl)amino]benzimidazol-1-yl]-2-[(3S)-pyrrolidin-3-yl]oxy-3-pyridyl]ethanol;
1-[3-(1-hydroxyethyl)-6-[6-[(2-oxopyrrolidin-1-yl)methyl]benzimidazol-1-yl]-2-pyridyl]-5-methyl-pyrazole-3-carbonitrile;
1-[3-(1-hydroxyethyl)-6-[5-[(2-oxopyrrolidin-1-yl)methyl]benzimidazol-1-yl]-2-pyridyl]-5-methyl-pyrazole-3-carbonitrile;
1-[2-(3-methoxy-5-methyl-pyrazol-1-yl)-6-[5-[(6-methylpyridazin-3-yl)amino]benzimidazol-1-yl]-3-pyridyl]ethanol;
1-[3-(1-hydroxyethyl)-6-[6-[(2-oxo-1-piperidyl)methyl]benzimidazol-1-yl]-2-pyridyl]-5-methyl-pyrazole-3-carbonitrile;
1-[3-(1-hydroxyethyl)-6-[5-[(2-oxo-1-piperidyl)methyl]benzimidazol-1-yl]-2-pyridyl]-5-methyl-pyrazole-3-carbonitrile;
3-[[1-[6-(3-cyano-5-methyl-pyrazol-1-yl)-5-(1-hydroxyethyl)-2-pyridyl]benzimidazol-5-yl]amino]-N,N,6-trimethyl-pyridazine-4-carboxamide;
5-hydroxy-2-[3-(1-hydroxyethyl)-6-[5-[(6-methylpyridazin-3-yl)amino]benzimidazol-1-yl]-2-pyridyl]benzonitrile;
5-[3-(1-hydroxyethyl)-6-[5-[(6-methylpyridazin-3-yl)amino]benzimidazol-1-yl]-2-pyridyl]-3-(trifluoromethyl)-1H-pyridazin-6-one;
2-[3-(1-hydroxyethyl)-6-[5-[(6-methylpyridazin-3-yl)amino]benzimidazol-1-yl]-2-pyridyl]furan-3-carbonitrile;
1-[2-[2-(2,2-difluorocyclopropyl)pyrazol-3-yl]-6-[5-[(6-methylpyridazin-3-yl)amino]benzimidazol-1-yl]-3-pyridyl]ethanol;
1-[3-(1-hydroxyethyl)-6-[5-[[6-(oxetan-3-yl)pyridazin-3-yl]amino]benzimidazol-1-yl]-2-pyridyl]-5-methyl-pyrazole-3-carbonitrile;
(1S)-1-[2-[3-(difluoromethyl)-5-methyl-pyrazol-1-yl]-6-[5-[(6-methylpyridazin-3-yl)amino]benzimidazol-1-yl]-3-pyridyl]ethanol;
(1R)-1-[2-[3-(difluoromethyl)-5-methyl-pyrazol-1-yl]-6-[5-[(6-methylpyridazin-3-yl)amino]benzimidazol-1-yl]-3-pyridyl]ethanol;
1-[2-[5-(difluoromethyl)-3-methyl-pyrazol-1-yl]-6-[5-[(6-methylpyridazin-3-yl)amino]benzimidazol-1-yl]-3-pyridyl]ethanol;
1-[3-(1-hydroxy-2-methoxy-ethyl)-6-[5-[(6-methylpyridazin-3-yl)amino]benzimidazol-1-yl]-2-pyridyl]-5-methyl-pyrazole-3-carbonitrile;
1-[3-(1-hydroxy-2-methoxy-ethyl)-6-[6-[(6-methylpyridazin-3-yl)amino]benzimidazol-1-yl]-2-pyridyl]-5-methyl-pyrazole-3-carbonitrile;
1-[6-[6-fluoro-5-[(6-methylpyridazin-3-yl)amino]benzimidazol-1-yl]-3-[(1S)-1-hydroxyethyl]-2-pyridyl]-5-methyl-pyrazole-3-carbonitrile;
1-[6-[6-fluoro-5-[(6-methylpyridazin-3-yl)amino]benzimidazol-1-yl]-3-[(1R)-1-hydroxyethyl]-2-pyridyl]-5-methyl-pyrazole-3-carbonitrile;
1-[5-chloro-3-(1-hydroxyethyl)-6-[5-[(6-methylpyridazin-3-yl)amino]benzimidazol-1-yl]-2-pyridyl]pyrazole-3-carbonitrile;
1-[6-[5-[(6-methylpyridazin-3-yl)amino]benzimidazol-1-yl]-2-[5-methyl-1-(2,2,2-trifluoroethyl)pyrazol-4-yl]-3-pyridyl]ethanol;
1-[6-[5-[(6-chloro-4-methoxy-pyridazin-3-yl)amino]benzimidazol-1-yl]-3-(1-hydroxyethyl)-2-pyridyl]-5-methyl-pyrazole-3-carbonitrile;
rac-(3R,5S)-1-[3-(1-hydroxyethyl)-6-[5-[(6-methylpyridazin-3-yl)amino]benzimidazol-1-yl]-2-pyridyl]-5-methyl-pyrrolidine-3-carbonitrile;
1-[5-chloro-3-(1-hydroxyethyl)-6-[5-[(6-methylpyridazin-3-yl)amino]benzimidazol-1-yl]-2-pyridyl]-5-methyl-pyrazole-3-carbonitrile;
5-[3-(1-hydroxyethyl)-6-[5-[(6-methylpyridazin-3-yl)amino]benzimidazol-1-yl]-2-pyridyl]-2-methyl-pyrazole-3-carbonitrile;
1-[3-[(1R)-1-hydroxyethyl]-6-[6-[(6-methylpyridazin-3-yl)amino]benzimidazol-1-yl]-2-pyridyl]-5-methyl-pyrazole-3-carbonitrile;
1-[2-[3,5-bis(difluoromethyl)pyrazol-1-yl]-6-[5-[(6-methylpyridazin-3-yl)amino]benzimidazol-1-yl]pyridin-3-yl]ethanol;
1-[3-(1-hydroxyethyl)-6-[5-[[6-(trifluoromethyl)pyridazin-3-yl]amino]benzimidazol-1-yl]-2-pyridyl]-5-methyl-pyrazole-3-carbonitrile;
1-[2-[2-ethyl-5-(trifluoromethyl)pyrazol-3-yl]-6-[5-[(6-methylpyridazin-3-yl)amino]benzimidazol-1-yl]-3-pyridyl]ethanol;
rac-(1S)-1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanol;
rac-(1R)-1-[6-[5-[(6-methylpyridazin-3-yl)amino]benzimidazol-1-yl]-2-[3-methyl-1-(2,2,2-trifluoroethyl)pyrazol-4-yl]-3-pyridyl]ethanol;
1-[3-(1-hydroxyethyl)-6-[5-[(6-methylpyridazin-3-yl)amino]benzimidazol-1-yl]-2-pyridyl]triazole-4-carbonitrile;
(3S,5R)-1-[3-(1-hydroxyethyl)-6-[5-[(6-methylpyridazin-3-yl)amino]benzimidazol-1-yl]-2-pyridyl]-5-methyl-pyrrolidine-3-carbonitrile;
5-[3-(1-hydroxyethyl)-6-[5-[(6-methylpyridazin-3-yl)amino]benzimidazol-1-yl]-2-pyridyl]-1-methyl-pyrazole-3-carbonitrile;
2-[3-(1-hydroxyethyl)-6-[5-[(6-methylpyridazin-3-yl)amino]benzimidazol-1-yl]-2-pyridyl]triazole-4-carbonitrile;
1-[6-(3-cyano-5-methylpyrazol-1-yl)-5-(1-hydroxyethyl)pyridin-2-yl]benzimidazole-5-carbonitrile;
1-[2-(1-ethyl-3-methyl-pyrazol-4-yl)-6-[5-[(6-methylpyridazin-3-yl)amino]benzimidazol-1-yl]-3-pyridyl]ethanol;
1-[2-[1-(2-methoxyethyl)-3-methyl-pyrazol-4-yl]-6-[5-[(6-methylpyridazin-3-yl)amino]benzimidazol-1-yl]-3-pyridyl]ethanol;
1-[2-[1-(cyclopropylmethyl)-3-methyl-pyrazol-4-yl]-6-[5-[(6-methylpyridazin-3-yl)amino]benzimidazol-1-yl]-3-pyridyl]ethanol;
5-methyl-1-[3-methylol-6-[6-[(6-methylpyridazin-3-yl)amino]benzimidazol-1-yl]-2-pyridyl]pyrazole-3-carbonitrile;
5-methyl-1-[3-methylol-6-[5-[(6-methylpyridazin-3-yl)amino]benzimidazol-1-yl]-2-pyridyl]pyrazole-3-carbonitrile;
1-[2-(1-ethyl-5-methyl-pyrazol-4-yl)-6-[5-[(6-methylpyridazin-3-yl)amino]benzimidazol-1-yl]-3-pyridyl]ethanol;
1-[2-[1-(cyclopropylmethyl)-5-methyl-pyrazol-4-yl]-6-[5-[(6-methylpyridazin-3-yl)amino]benzimidazol-1-yl]-3-pyridyl]ethanol;
1-[2-[1-(cyclobutylmethyl)-3-methyl-pyrazol-4-yl]-6-[5-[(6-methylpyridazin-3-yl)amino]benzimidazol-1-yl]-3-pyridyl]ethanol;
1-[2-[1-(cyclobutylmethyl)-5-methyl-pyrazol-4-yl]-6-[5-[(6-methylpyridazin-3-yl)amino]benzimidazol-1-yl]-3-pyridyl]ethanol;
1-[3-(1-hydroxyethyl)-6-[5-[(6-methylpyridazin-3-yl)amino]-6-(trifluoromethyl)benzimidazol-1-yl]-2-pyridyl]-5-methyl-pyrazole-3-carbonitrile;
1-[3-[(1S)-1-hydroxyethyl]-6-[5-[(6-methylpyridazin-3-yl)amino]benzimidazol-1-yl]-2-pyridyl]-5-methyl-pyrazole-3-carboximidic acid methyl ester;
1-[2-[1-(2,2-difluorocyclopropyl)-3-methyl-pyrazol-4-yl]-6-[5-[(6-methylpyridazin-3-yl)amino]benzimidazol-1-yl]-3-pyridyl]ethanol;
1-[2-[1-(2-methoxypropyl)-3-methyl-pyrazol-4-yl]-6-[5-[(6-methylpyridazin-3-yl)amino]benzimidazol-1-yl]-3-pyridyl]ethanol;
1-[2-[1-(2-methoxypropyl)-5-methyl-pyrazol-4-yl]-6-[5-[(6-methylpyridazin-3-yl)amino]benzimidazol-1-yl]-3-pyridyl]ethanol;
1-[3-[(1S)-1-hydroxyethyl]-6-[6-[(6-methylpyridazin-3-yl)amino]benzimidazol-1-yl]-2-pyridyl]-5-methyl-pyrazole-3-carbonitrile;
1-[2-[1-(2,2-difluoropropyl)-3-methyl-pyrazol-4-yl]-6-[5-[(6-methylpyridazin-3-yl)amino]benzimidazol-1-yl]-3-pyridyl]ethanol;
1-[3-(1-hydroxyethyl)-6-[5-[(6-methylpyridazin-3-yl)amino]-6-(oxetan-3-yloxy)benzimidazol-1-yl]-2-pyridyl]-5-methyl-pyrazole-3-carbonitrile;
1-[2-(2-ethyl-5-methyl-4-pyridyl)-6-[5-[(6-methylpyridazin-3-yl)amino]benzimidazol-1-yl]-3-pyridyl]ethanol;
1-[2-[1-(2,2-difluoropropyl)-5-methyl-pyrazol-4-yl]-6-[5-[(6-methylpyridazin-3-yl)amino]benzimidazol-1-yl]-3-pyridyl]ethanol;
1-[2-(2-methoxy-5-methyl-4-pyridyl)-6-[5-[(6-methylpyridazin-3-yl)amino]benzimidazol-1-yl]-3-pyridyl]ethanol;
(1S)-1-[2-[3,5-bis(difluoromethyl)pyrazol-1-yl]-6-[5-[(6-methylpyridazin-3-yl)amino]benzimidazol-1-yl]-3-pyridyl]ethanol;
(1R)-1-[2-[3,5-bis(difluoromethyl)pyrazol-1-yl]-6-[5-[(6-methylpyridazin-3-yl)amino]benzimidazol-1-yl]-3-pyridyl]ethanol;
1-[2-(2-cyclopropyl-5-methyl-4-pyridyl)-6-[5-[(6-methylpyridazin-3-yl)amino]benzimidazol-1-yl]-3-pyridyl]ethanol;
1-[2-[3-(difluoromethoxy)-5-methyl-pyrazol-1-yl]-6-[5-[(6-methylpyridazin-3-yl)amino]benzimidazol-1-yl]-3-pyridyl]ethanol;
1-[3-(1-hydroxyethyl)-6-[6-[(6-methylpyridazin-3-yl)amino]benzimidazol-1-yl]-2-pyridyl]-5-methyl-6,7-dihydro-4˜{H}-pyrazolo[4,3-c]pyridine-3-carbonitrile;
1-[3-(1-hydroxyethyl)-6-[5-[(6-methyl-4-methylsulfonyl-pyridazin-3-yl)amino]benzimidazol-1-yl]-2-pyridyl]-5-methyl-pyrazole-3-carbonitrile;
1-[3-(1-hydroxyethyl)-6-[5-[[6-[2-[2-[2-[2-[2-[2-(2-methoxyethoxy)ethoxy]ethoxy]ethoxy]ethoxy]ethoxy]ethoxy]pyridazin-3-yl]amino]benzimidazol-1-yl]-2-pyridyl]-5-methyl-pyrazole-3-carbonitrile;
1-[3-(1-hydroxyethyl)-6-[6-[(6-methyl-3-pyridyl)amino]-5-(2-morpholinoethoxy)benzimidazol-1-yl]-2-pyridyl]-5-methyl-pyrazole-3-carbonitrile;
1-[3-(1-hydroxyethyl)-6-[6-(2-oxa-5-azaspiro[3.4]octan-5-yl)benzimidazol-1-yl]-2-pyridyl]-5-methyl-pyrazole-3-carbonitrile;
1-[3-(1-hydroxyethyl)-6-[5-(2-oxa-5-azaspiro[3.4]octan-5-yl)benzimidazol-1-yl]-2-pyridyl]-5-methyl-pyrazole-3-carbonitrile;
1-[3-(1-hydroxyethyl)-6-[5-[(6-methyl-3-pyridyl)amino]-6-(2-morpholinoethoxy)benzimidazol-1-yl]-2-pyridyl]-5-methyl-pyrazole-3-carbonitrile;
1-[2-[3-(difluoromethyl)pyrrolidin-1-yl]-6-[5-[(6-methylpyridazin-3-yl)amino]benzimidazol-1-yl]-3-pyridyl]ethanol;
1-[2-(2,2-difluoro-5-azaspiro[2.4]heptan-5-yl)-6-[5-[(6-methylpyridazin-3-yl)amino]benzimidazol-1-yl]-3-pyridyl]ethanol;
(1S)-1-[2-[3-(difluoromethyl)-5-methyl-pyrazol-1-yl]-6-[6-fluoro-5-[(6-methylpyridazin-3-yl)amino]benzimidazol-1-yl]-3-pyridyl]ethanol;
1-[6-[6-fluoro-5-[(6-methylpyridazin-3-yl)amino]benzimidazol-1-yl]-2-[3-methyl-1-(2,2,2-trifluoroethyl)pyrazol-4-yl]-3-pyridyl]ethanol;
5-(difluoromethyl)-2-[3-(1-hydroxyethyl)-6-[5-[(6-methylpyridazin-3-yl)amino]benzimidazol-1-yl]-2-pyridyl]pyrazole-3-carbonitrile;
1-[3-(1-hydroxyethyl)-6-[5-[[6-[1-(oxetan-3-yl)pyrrolidin-2-yl]pyridazin-3-yl]amino]benzimidazol-1-yl]-2-pyridyl]-5-methyl-pyrazole-3-carbonitrile;
7,7-difluoro-1-[3-(1-hydroxyethyl)-6-[5-[(6-methylpyridazin-3-yl)amino]benzimidazol-1-yl]-2-pyridyl]-5,6-dihydro-4˜{H}-pyrazolo[4,3-c]pyridine-3-carbonitrile;
5-(difluoromethyl)-1-[3-(1-hydroxyethyl)-6-[5-[(6-methylpyridazin-3-yl)amino]benzimidazol-1-yl]-2-pyridyl]pyrazole-3-carbonitrile;
(1S)-1-[2-[3-(difluoromethyl)-5-methyl-pyrazol-1-yl]-6-[6-fluoro-5-(pyridazin-3-ylamino)benzimidazol-1-yl]-3-pyridyl]ethanol;
1-[6-[5-[(6-methylpyridazin-3-yl)amino]benzimidazol-1-yl]-2-[5-methyl-2-(2,2,2-trifluoroethyl)triazol-4-yl]-3-pyridyl]ethanol;
1-[3-(1-hydroxyethyl)-6-[5-[(6-methylpyridazin-3-yl)amino]benzimidazol-1-yl]-2-pyridyl]-4,5-dimethyl-pyrazole-3-carbonitrile;
1-[3-(1-hydroxyethyl)-6-[5-[(4-methyl-2,3-dihydropyridazino[4,5-b][1,4]oxazin-8-yl)amino]benzimidazol-1-yl]-2-pyridyl]-5-methyl-pyrazole-3-carbonitrile;
1-[6-[5-[(6-methylpyridazin-3-yl)amino]benzimidazol-1-yl]-2-[5-methyl-1-(2,2,2-trifluoroethyl)triazol-4-yl]-3-pyridyl]ethanol;
1-[1-[6-(3-cyano-5-methyl-pyrazol-1-yl)-5-(1-hydroxyethyl)-2-pyridyl]benzimidazol-5-yl]-N,N-dimethyl-pyrrolidine-2-carboxamide;
1-[2-[1-(2,2-difluoroethyl)-3-methyl-pyrazol-4-yl]-6-[5-[(6-methylpyridazin-3-yl)amino]benzimidazol-1-yl]-3-pyridyl]ethanol;
1-[6-[5-fluoro-6-[(6-methylpyridazin-3-yl)amino]benzimidazol-1-yl]-3-(hydroxymethyl)-2-pyridyl]-5-methyl-pyrazole-3-carbonitrile;
[6-[5-[(6-methylpyridazin-3-yl)amino]benzimidazol-1-yl]-2-[3-methyl-1-(2,2,2-trifluoroethyl)pyrazol-4-yl]-3-pyridyl]methanol;
[6-[5-[(6-methylpyridazin-3-yl)amino]benzimidazol-1-yl]-2-[5-methyl-1-(2,2,2-trifluoroethyl)pyrazol-4-yl]-3-pyridyl]methanol;
[2-[3-(difluoromethyl)-5-methyl-pyrazol-1-yl]-6-[5-(7,8-dihydro-5H-pyrano[4,3-c]pyridazin-3-ylamino)benzimidazol-1-yl]-3-pyridyl]methanol;
[6-[5-[(4aS,7aR)-4-methyl-2,3,4a,5,7,7a-hexahydropyrrolo[3,4-b][1,4]oxazin-6-yl]benzimidazol-1-yl]-2-[3-(difluoromethyl)-5-methyl-pyrazol-1-yl]-3-pyridyl]methanol;
[2-[3-(difluoromethyl)-5-methyl-pyrazol-1-yl]-6-[6-fluoro-5-[(6-methylpyridazin-3-yl)amino]benzimidazol-1-yl]-3-pyridyl]methanol;
1-[6-[5-[(6-methylpyridazin-3-yl)amino]benzimidazol-1-yl]-2-[2-(trifluoromethyl)morpholin-4-yl]-3-pyridyl]ethanol;
3-[3-(1-hydroxyethyl)-6-[5-[(6-methylpyridazin-3-yl)amino]benzimidazol-1-yl]-2-pyridyl]-3-azabicyclo[3.1.0]hexane-1-carbonitrile;
1-[2-(5,5-difluoro-2-azabicyclo[2.2.1]heptan-2-yl)-6-[5-[(6-methylpyridazin-3-yl)amino]benzimidazol-1-yl]-3-pyridyl]ethanol;
1-[2-[2-(difluoromethyl)morpholin-4-yl]-6-[5-[(6-methylpyridazin-3-yl)amino]benzimidazol-1-yl]-3-pyridyl]ethanol;
1-[2-(6,6-difluoro-2-azabicyclo[2.2.1]heptan-2-yl)-6-[5-[(6-methylpyridazin-3-yl)amino]benzimidazol-1-yl]-3-pyridyl]ethanol;
1-[2-[7-(difluoromethyl)-5-azaspiro[2.4]heptan-5-yl]-6-[5-[(6-methylpyridazin-3-yl)amino]benzimidazol-1-yl]-3-pyridyl]ethanol;
1-[2-(6,6-difluoro-3-azabicyclo[3.2.0]heptan-3-yl)-6-[5-[(6-methylpyridazin-3-yl)amino]benzimidazol-1-yl]-3-pyridyl]ethanol;
N-[1-[6-(3-cyano-5-methyl-pyrazol-1-yl)-5-(1-hydroxyethyl)-2-pyridyl]benzimidazol-5-yl]cyclopropanecarboxamide;
N-[3-[6-(3-cyano-5-methyl-pyrazol-1-yl)-5-(1-hydroxyethyl)-2-pyridyl]benzimidazol-5-yl]cyclopropanecarboxamide;
1-[2-(4,7-diazaspiro[2.5]octan-7-yl)-6-[5-[(6-methylpyridazin-3-yl)amino]benzimidazol-1-yl]-3-pyridyl]ethanol;
1-[6-[5-[(3aS,6aS)-6-oxo-2,3,3a,4,5,6a-hexahydropyrrolo[2,3-c]pyrrol-1-yl]benzimidazol-1-yl]-3-(1-hydroxyethyl)-2-pyridyl]-5-methyl-pyrazole-3-carbonitrile;
1-[6-[5-[(6-methylpyridazin-3-yl)amino]benzimidazol-1-yl]-2-[3-(trifluoromethyl)piperazin-1-yl]-3-pyridyl]ethanol;
1-[6-[5-[(6-methylpyridazin-3-yl)amino]benzimidazol-1-yl]-2-[5-methyl-2-(trifluoromethyl)-4-pyridyl]-3-pyridyl]ethanol;
1-[3-(1-hydroxyethyl)-6-[5-[(2-keto-1-methyl-3-pyridyl)amino]benzimidazol-1-yl]-2-pyridyl]-5-methyl-pyrazole-3-carbonitrile;
1-[3-(1-hydroxyethyl)-6-[5-(3-methoxy-1-methyl-pyrazol-4-yl)benzimidazol-1-yl]-2-pyridyl]-5-methyl-pyrazole-3-carbonitrile;
1-[2-[2-(difluoromethyl)-5-methyl-4-pyridyl]-6-[5-[(6-methylpyridazin-3-yl)amino]benzimidazol-1-yl]-3-pyridyl]ethanol;
1-[3-(1-hydroxyethyl)-6-[6-(1-methyl-2-oxo-pyrimidin-4-yl)benzimidazol-1-yl]-2-pyridyl]-5-methyl-pyrazole-3-carbonitrile;
1-[2-[3-(difluoromethyl)-5-methyl-pyrazol-1-yl]-6-[6-fluoro-5-[[6-(2-methyl-1,3-dioxolan-2-yl)pyridazin-3-yl]amino]benzimidazol-1-yl]-3-pyridyl]ethanol;
1-[3-(1-hydroxyethyl)-6-[5-(1-methyl-2-oxo-pyrimidin-4-yl)benzimidazol-1-yl]-2-pyridyl]-5-methyl-pyrazole-3-carbonitrile;
1-[3-(1-hydroxyethyl)-6-[6-(6-methylpyridazin-3-yl)benzimidazol-1-yl]-2-pyridyl]-5-methyl-pyrazole-3-carbonitrile;
1-[3-(1-hydroxyethyl)-6-[5-(6-methylpyridazin-3-yl)benzimidazol-1-yl]-2-pyridyl]-5-methyl-pyrazole-3-carbonitrile;
1-[6-[6-[(2S)-2-cyanopyrrolidin-1-yl]benzimidazol-1-yl]-3-(1-hydroxyethyl)-2-pyridyl]-5-methyl-pyrazole-3-carbonitrile;
1-[6-[5-[(2S)-2-cyanopyrrolidin-1-yl]benzimidazol-1-yl]-3-(1-hydroxyethyl)-2-pyridyl]-5-methyl-pyrazole-3-carbonitrile;
1-[6-[6-[(3aS,6aS)-6-oxo-2,3,3a,4,5,6a-hexahydropyrrolo[2,3-c]pyrrol-1-yl]benzimidazol-1-yl]-3-(1-hydroxyethyl)-2-pyridyl]-5-methyl-pyrazole-3-carbonitrile;
1-[2-[3-(difluoromethyl)-5-methyl-1,2,4-triazol-1-yl]-6-[5-[(6-methylpyridazin-3-yl)amino]benzimidazol-1-yl]-3-pyridyl]ethanol;
3-[[1-[6-[3-(difluoromethyl)-5-methyl-pyrazol-1-yl]-5-[(1˜{S})-1-hydroxyethyl]-2-pyridyl]-6-fluoro-benzimidazol-5-yl]amino]-˜{N},˜{N},6-trimethyl-pyridazine-4-carboxamide;
1-[2-[2-(difluoromethoxy)-5-methyl-4-pyridyl]-6-[6-fluoro-5-[(6-methylpyridazin-3-yl)amino]benzimidazol-1-yl]-3-pyridyl]ethanol;
1-[6-[6-fluoro-5-[(6-methylpyridazin-3-yl)amino]benzimidazol-1-yl]-3-(hydroxymethyl)-2-pyridyl]-5-methyl-pyrazole-3-carbonitrile;
3-[[1-[6-[3,5-bis(difluoromethyl)pyrazol-1-yl]-5-(hydroxymethyl)-2-pyridyl]benzimidazol-5-yl]amino]-N,N,6-trimethyl-pyridazine-4-carboxamide;
1-[6-[5-[(6-methylpyridazin-3-yl)amino]benzimidazol-1-yl]-2-[4-methyl-1-(2,2,2-trifluoroethyl)pyrazol-3-yl]-3-pyridyl]ethanol;
1-[2-[3-methyl-1-(2,2,2-trifluoroethyl)pyrazol-4-yl]-6-[5-[6-(morpholinomethyl)pyridazin-3-yl]oxybenzimidazol-1-yl]-3-pyridyl]ethanol;
1-[6-[5-(6-methylpyridazin-3-yl)oxybenzimidazol-1-yl]-2-[3-methyl-1-(2,2,2-trifluoroethyl)pyrazol-4-yl]-3-pyridyl]ethanol;
1-[6-[5-[6-[(4-methylpiperazin-1-yl)methyl]pyridazin-3-yl]oxybenzimidazol-1-yl]-2-[3-methyl-1-(2,2,2-trifluoroethyl)pyrazol-4-yl]-3-pyridyl]ethanol;
1-[2-[3-(difluoromethoxy)-5-methyl-pyrazol-1-yl]-6-[6-[(6-methylpyridazin-3-yl)amino]-5-(oxetan-3-yloxy)benzimidazol-1-yl]-3-pyridyl]ethanol;
1-[2-(3-chloro-5-methyl-pyrazol-1-yl)-6-[5-fluoro-6-[(6-methylpyridazin-3-yl)amino]benzimidazol-1-yl]-3-pyridyl]ethanol;
1-[2-(3-chloro-5-methyl-pyrazol-1-yl)-6-[6-fluoro-5-[(6-methylpyridazin-3-yl)amino]benzimidazol-1-yl]-3-pyridyl]ethanol;
1-[2-[1-(cyclopropylmethyl)-3-methyl-pyrazol-4-yl]-6-[6-fluoro-5-[(6-methylpyridazin-3-yl)amino]benzimidazol-1-yl]-3-pyridyl]ethanol;
1-[2-[3-(difluoromethoxy)-5-methyl-pyrazol-1-yl]-6-[5-[(6-methylpyridazin-3-yl)amino]-6-(oxetan-3-yloxy)benzimidazol-1-yl]-3-pyridyl]ethanol;
N-[1-[5-(1-hydroxyethyl)-6-[3-methyl-1-(2,2,2-trifluoroethyl)pyrazol-4-yl]-2-pyridyl]benzimidazol-5-yl]-2-(1-piperidyl)acetamide;
6-[3-(1-hydroxyethyl)-6-[5-[(6-methylpyridazin-3-yl)amino]benzimidazol-1-yl]-2-pyridyl]-6-azaspiro[3.4]octane-8-carbonitrile;
4-[3-(1-hydroxyethyl)-6-[5-[(6-methylpyridazin-3-yl)amino]benzimidazol-1-yl]-2-pyridyl]morpholine-2-carbonitrile;
N-[1-[6-[3-(difluoromethyl)-5-methyl-pyrazol-1-yl]-5-(1-hydroxyethyl)-2-pyridyl]-6-fluoro-benzimidazol-5-yl]cyclopropanecarboxamide;
1-[6-[6-(2,3-difluoro-4-methyl-anilino)-5-fluoro-benzimidazol-1-yl]-2-[3-(difluoromethyl)-5-methyl-pyrazol-1-yl]-3-pyridyl]ethanol;
1-[2-[i-(difluoromethyl)-4-methyl-pyrazol-3-yl]-6-[5-[(6-methylpyridazin-3-yl)amino]benzimidazol-1-yl]-3-pyridyl]ethanol;
1-[2-[3-(difluoromethyl)-5-methyl-pyrazol-1-yl]-6-[5-fluoro-6-(2,3,4-trifluoroanilino)benzimidazol-1-yl]-3-pyridyl]ethanol;
1-[3-(1-hydroxyethyl)-6-[6-(6-methyl-7-oxo-5H-pyrrolo[3,4-b]pyridin-2-yl)benzimidazol-1-yl]-2-pyridyl]-5-methyl-pyrazole-3-carbonitrile;
1-[3-(1-hydroxyethyl)-6-[5-(6-methyl-7-oxo-5H-pyrrolo[3,4-b]pyridin-2-yl)benzimidazol-1-yl]-2-pyridyl]-5-methyl-pyrazole-3-carbonitrile;
1-[2-[3-(difluoromethoxy)-5-methylpyrazol-1-yl]-6-[5-[[6-(difluoromethyl)pyridazin-3-yl]amino]benzimidazol-1-yl]pyridin-3-yl]ethanol;
1-[2-[3-(difluoromethyl)-5-ethyl-pyrazol-1-yl]-6-[6-[(6-methylpyridazin-3-yl)amino]benzimidazol-1-yl]-3-pyridyl]ethanol;
1-[2-[3-(difluoromethyl)-4,5-dimethyl-pyrazol-1-yl]-6-[5-[(6-methylpyridazin-3-yl)amino]benzimidazol-1-yl]-3-pyridyl]ethanol;
1-[2-[3-(difluoromethyl)-5-ethyl-pyrazol-1-yl]-6-[5-[(6-methylpyridazin-3-yl)amino]benzimidazol-1-yl]-3-pyridyl]ethanol;
1-[6-[6-[(6-chloropyridazin-3-yl)amino]-5-fluoro-benzimidazol-1-yl]-2-[3-(difluoromethyl)-5-methyl-pyrazol-1-yl]-3-pyridyl]ethanol;
1-[6-[5-[(6-chloropyridazin-3-yl)amino]-6-fluoro-benzimidazol-1-yl]-2-[3-(difluoromethyl)-5-methyl-pyrazol-1-yl]-3-pyridyl]ethanol;
(1S)-1-[2-[3-(difluoromethoxy)-5-methyl-pyrazol-1-yl]-6-[6-fluoro-5-(pyridazin-3-ylamino)benzimidazol-1-yl]-3-pyridyl]ethanol;
(1S)-1-[2-[3-(difluoromethoxy)-5-methyl-pyrazol-1-yl]-6-[6-fluoro-5-[(6-methylpyridazin-3-yl)amino]benzimidazol-1-yl]-3-pyridyl]ethanol;
(1R)-1-[2-[3-(difluoromethoxy)-5-methyl-pyrazol-1-yl]-6-[6-fluoro-5-[(6-methylpyridazin-3-yl)amino]benzimidazol-1-yl]-3-pyridyl]ethanol;
1-[6-[5-fluoro-6-[(5-methyl-1,3,4-oxadiazol-2-yl)amino]benzimidazol-1-yl]-3-(1-hydroxyethyl)-2-pyridyl]-5-methyl-pyrazole-3-carbonitrile;
1-[6-[6-fluoro-5-[(5-methyl-1,3,4-oxadiazol-2-yl)amino]benzimidazol-1-yl]-3-(1-hydroxyethyl)-2-pyridyl]-5-methyl-pyrazole-3-carbonitrile;
1-[6-[6-fluoro-5-(2,3,4-trifluoroanilino)benzimidazol-1-yl]-3-(1-hydroxyethyl)-2-pyridyl]-5-methyl-pyrazole-3-carbonitrile;
1-[6-[5-fluoro-6-(2,3,4-trifluoroanilino)benzimidazol-1-yl]-3-(1-hydroxyethyl)-2-pyridyl]-5-methyl-pyrazole-3-carbonitrile;
N-[1-[6-(3-cyano-5-methyl-pyrazol-1-yl)-5-(1-hydroxyethyl)-2-pyridyl]benzimidazol-5-yl]-1-fluoro-cyclopropanecarboxamide;
1-[6-[6-(6,7-dihydro-5˜{H}-cyclopenta[c]pyridazin-3-ylamino)-5-fluoro-benzimidazol-1-yl]-3-(1-hydroxyethyl)-2-pyridyl]-5-methyl-pyrazole-3-carbonitrile;
1-[6-[5-(6,7-dihydro-5˜{H}-cyclopenta[c]pyridazin-3-ylamino)-6-fluoro-benzimidazol-1-yl]-3-(1-hydroxyethyl)-2-pyridyl]-5-methyl-pyrazole-3-carbonitrile;
(1S)-1-[2-[3-(difluoromethoxy)-5-methyl-pyrazol-1-yl]-6-[5-[(6-methylpyridazin-3-yl)amino]-6-(oxetan-3-yloxy)benzimidazol-1-yl]-3-pyridyl]ethanol;
(1R)-1-[2-[3-(difluoromethoxy)-5-methyl-pyrazol-1-yl]-6-[5-[(6-methylpyridazin-3-yl)amino]-6-(oxetan-3-yloxy)benzimidazol-1-yl]-3-pyridyl]ethanol;
1-[6-[6-fluoro-5-[(5-methyl-1,3,4-thiadiazol-2-yl)amino]benzimidazol-1-yl]-3-(1-hydroxyethyl)-2-pyridyl]-5-methyl-pyrazole-3-carbonitrile;
3-fluoro-4-[3-(1-hydroxyethyl)-6-[5-[(6-methylpyridazin-3-yl)amino]benzimidazol-1-yl]-2-pyridyl]-1-(2,2,2-trifluoroethyl)pyridin-2-one;
1-[2-[3,5-bis(difluoromethyl)pyrazol-1-yl]-6-[6-fluoro-5-[(6-methylpyridazin-3-yl)amino]benzimidazol-1-yl]-3-pyridyl]ethanol;
1-[6-[6-(difluoromethoxy)-5-[(6-methylpyridazin-3-yl)amino]benzimidazol-1-yl]-3-(1-hydroxyethyl)-2-pyridyl]-5-methyl-pyrazole-3-carbonitrile;
1-[6-[6-fluoro-5-[(5-fluoro-6-methyl-3-pyridyl)amino]benzimidazol-1-yl]-3-(1-hydroxyethyl)-2-pyridyl]-5-methyl-pyrazole-3-carbonitrile;
1-[6-[5-[[5-(azetidin-1-yl)pyridazin-3-yl]amino]-6-fluoro-benzimidazol-1-yl]-2-[3-(difluoromethyl)-5-methyl-pyrazol-1-yl]-3-pyridyl]ethanol;
1-[6-[6-fluoro-5-[(5-fluoro-6-methyl-3-pyridyl)amino]benzimidazol-1-yl]-3-[(1S)-1-hydroxyethyl]-2-pyridyl]-5-methyl-pyrazole-3-carbonitrile;
1-[3-(1-hydroxyethyl)-6-[5-[2-(2-oxopyrrolidin-1-yl)oxazol-5-yl]benzimidazol-1-yl]-2-pyridyl]-5-methyl-pyrazole-3-carbonitrile;
1-[2-(4,4-difluoropyrrolidin-3-yl)oxy-6-[5-[(6-methylpyridazin-3-yl)amino]benzimidazol-1-yl]-3-pyridyl]ethanol;
1-[6-[5-(7,8-dihydro-5˜{H}-pyrano[4,3-c]pyridazin-3-ylamino)-6-methoxy-benzimidazol-1-yl]-3-(1-hydroxyethyl)-2-pyridyl]-5-methyl-pyrazole-3-carbonitrile;
1-[3-(1-hydroxyethyl)-6-[6-[2-(2-oxopyrrolidin-1-yl)oxazol-5-yl]benzimidazol-1-yl]-2-pyridyl]-5-methyl-pyrazole-3-carbonitrile;
1-[2-[6-(difluoromethoxy)-3-methyl-pyridazin-4-yl]-6-[5-[(6-methylpyridazin-3-yl)amino]benzimidazol-1-yl]-3-pyridyl]ethanol;
1-[2-[3-(difluoromethyl)-5-methoxy-pyrazol-1-yl]-6-[6-[(6-methylpyridazin-3-yl)amino]benzimidazol-1-yl]-3-pyridyl]ethanol;
1-[2-[3-(difluoromethyl)-5-methoxy-pyrazol-1-yl]-6-[5-[(6-methylpyridazin-3-yl)amino]benzimidazol-1-yl]-3-pyridyl]ethanol;
1-[2-[1-(2,3-difluoropropyl)-3-methyl-pyrazol-4-yl]-6-[5-[(6-methylpyridazin-3-yl)amino]benzimidazol-1-yl]-3-pyridyl]ethanol;
(1S)-1-[2-[3-(difluoromethoxy)-5-methyl-pyrazol-1-yl]-6-[6-methoxy-5-[(2-methylpyrimidin-5-yl)amino]benzimidazol-1-yl]-3-pyridyl]ethanol;
1-[6-[5-[(6-methylpyridazin-3-yl)amino]benzimidazol-1-yl]-2-[2-methyl-5-(2,2,2-trifluoroethyl)-1,2,4-triazol-3-yl]-3-pyridyl]ethanol;
1-[6-[6-(difluoromethoxy)-5-[(6-methylpyridazin-3-yl)amino]benzimidazol-1-yl]-3-[(1S)-1-hydroxyethyl]-2-pyridyl]-5-methyl-pyrazole-3-carbonitrile;
1-[6-[6-(difluoromethoxy)-5-[(6-methylpyridazin-3-yl)amino]benzimidazol-1-yl]-3-[(1R)-1-hydroxyethyl]-2-pyridyl]-5-methyl-pyrazole-3-carbonitrile;
1-[2-[3-(difluoromethoxy)-5-(difluoromethyl)pyrazol-1-yl]-6-[5-[(6-methylpyridazin-3-yl)amino]benzimidazol-1-yl]-3-pyridyl]ethanol;
1-[2-[3-(difluoromethoxy)-5-(difluoromethyl)pyrazol-1-yl]-6-[6-[(6-methylpyridazin-3-yl)amino]benzimidazol-1-yl]-3-pyridyl]ethanol;
1-[2-[3-(difluoromethoxy)-5-methyl-pyrazol-1-yl]-6-[6-[(6-methylpyridazin-3-yl)amino]-5-(oxetan-3-ylmethyl)benzimidazol-1-yl]-3-pyridyl]ethanol; and
1-[2-[3-(difluoromethoxy)-5-methyl-pyrazol-1-yl]-6-[5-[(6-methylpyridazin-3-yl)amino]-6-(oxetan-3-ylmethyl)benzimidazol-1-yl]-3-pyridyl]ethanol;
or a pharmaceutically acceptable salt thereof.

12. A process for the preparation of a compound according to claim 1, comprising one of the following steps:
(a) the reaction of a compound of formula (B1) 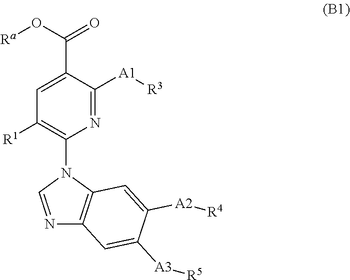
with a reducing agent;
(b) the reaction of a compound of formula (C1) 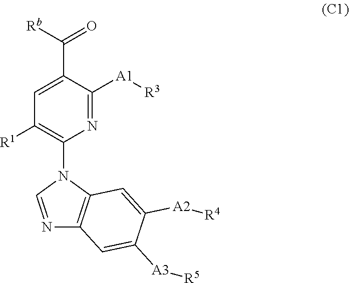
with a reducing agent; or
(c) the reaction of a compound (D1) 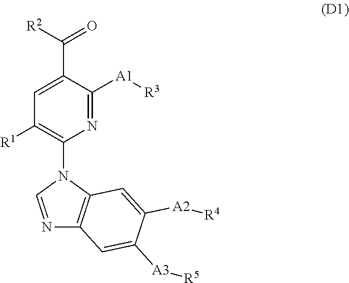
with a compound of formula R^{c}MgX,
wherein A1, A2, A3, R¹, R², R³, R⁴ and R⁵ are as defined in claim 1, R^{a} is alkyl or cycloalkyl, R^{b} is hydrogen or alkyl, R^{c} is alkyl or cycloalkyl, and X is halogen.

13. A pharmaceutical composition comprising a compound of formula (I) according to claim 1 or a pharmaceutically acceptable salt thereof, and a therapeutically inert carrier.

14. A method for the treatment or prophylaxis of rheumatoid arthritis, juvenile rheumatoid arthritis, non-alcoholic steatohepatitis (NASH), primary sclerosing cholangitis, giant cell vasculitis, inflammatory bowel diseases (IBD), atherosclerosis, type 2 diabetes or glomerulonephritis, which method comprises administering an effective amount of a compound of formula (I) according to claim 1 or a pharmaceutically acceptable salt thereof, to a patient in need thereof.
